(19) United States
(12) Patent Application Publication
(10) Pub. No.: US 2022/0333131 A1
(43) Pub. Date: Oct. 20, 2022

(54) MODULATORY POLYNUCLEOTIDES

(71) Applicant: VOYAGER THERAPEUTICS, INC., Cambridge, MA (US)

(72) Inventors: Jinzhao Hou, Lexington, MA (US); Xin Wang, Arlington, MA (US); Pengcheng Zhou, Lexington, MA (US); Xiao-Qin Ren, North Attleboro, MA (US); Dinah Wen-Yee Sah, Hopkinton, MA (US)

(21) Appl. No.: 17/561,252

(22) Filed: Dec. 23, 2021

Related U.S. Application Data
(63) Continuation of application No. 16/611,046, filed on Nov. 5, 2019, now abandoned.
(60) Provisional application No. 62/520,093, filed on Jun. 15, 2017.
(60) Provisional application No. 62/507,923, filed on May 18, 2017.
(60) Provisional application No. 62/501,787, filed on May 5, 2017.

(51) Int. Cl. C12N 15/86; C12N 7/00; C12N 15/113

(52) U.S. Cl. CPC C12N 15/86; C12N 7/00; C12N 15/113; C12N 2310/11; C12N 2310/531; C12N 2750/14143

(57) ABSTRACT

The present invention relates to adeno-associated viral (AAV) particles modulatory polynucleotides encoding at least one siRNA molecules and methods of use thereof.

### CROSS REFERENCE TO RELATED APPLICATIONS

This application is a continuation of U.S. patent application Ser. No. 16/611,046, filed Nov. 5, 2019, which is a U.S. National Stage Application under 35 U.S.C. § 371 of International Application No. PCT/US2018/031108, filed May 4, 2018, which claims the benefit of U.S. Provisional Patent Application No. 62/501,787, filed May 5, 2017, U.S. Provisional Patent Application No. 62/507,923, filed May 18, 2017, and U.S. Provisional Patent Application No. 62/520,093, filed Jun. 15, 2017, the contents of each of which is incorporated by reference herein in its entirety.

### REFERENCE TO THE SEQUENCE LISTING

The instant application contains a Sequence Listing which has been submitted electronically in ASCII format and is hereby incorporated by reference in its entirety. Said ASCII copy, created on Dec. 23, 2021 is named 2057_1045USCON_SL and is 6,814,412 bytes in size.

### FIELD OF THE INVENTION

The present invention relates to compositions, methods and processes for the design, preparation, manufacture, use and/or formulation of AAV particles comprising modulatory polynucleotides, e.g., polynucleotides encoding at least one small interfering RNA (siRNA) molecules which targets at least one gene of interest. Targeting the gene of interest may interfere with the gene expression and the resultant protein production. The AAV particles comprising modulatory polynucleotides encoding at least one siRNA molecules may be inserted into recombinant adeno-associated virus (AAV) vectors. Methods for using the AAV particles to inhibit the expression of the gene of interest in a subject are also disclosed.

### BACKGROUND OF THE INVENTION

MicroRNAs (or miRNAs or miRs) are small, non-coding, single stranded ribonucleic acid molecules (RNAs), which are usually 19-25 nucleotides in length. More than a thousand microRNAs have been identified in mammalian genomes. The mature microRNAs primarily bind to the 3′ untranslated region (3′-UTR) of target messenger RNAs (mRNAs) through partially or fully pairing with the complementary sequences of target mRNAs, promoting the degradation of target mRNAs at a post-transcriptional level, and in some cases, inhibiting the initiation of translation. MicroRNAs play a critical role in many key biological processes, such as the regulation of cell cycle and growth, apoptosis, cell proliferation and tissue development.

miRNA genes are generally transcribed as long primary transcripts of miRNAs (i.e. pri-miRNAs). The pri-miRNA is cleaved into a precursor of a miRNA (i.e. pre-miRNA) which is further processed to generate the mature and functional miRNA.

While many target expression strategies employ nucleic acid based modalities, there remains a need for improved nucleic acid modalities which have higher specificity and with fewer off target effects.

The present invention provides such improved modalities in the form of artificial pri-, pre- and mature microRNA constructs and methods of their design. These novel constructs may be synthetic stand-alone molecules or be encoded in a plasmid or expression vector for delivery to cells. Such vectors include, but are not limited to adeno-associated viral vectors such as vector genomes of any of the AAV serotypes or other viral delivery vehicles such as lentivirus, etc.

### SUMMARY OF THE INVENTION

Described herein are methods, processes, compositions, kits and devices for the administration of AAV particles comprising modulatory polynucleotides encoding at least one siRNA molecule for the treatment, prophylaxis, palliation and/or amelioration of a disease and/or disorder.

The details of various embodiments of the invention are set forth in the description below. Other features, objects, and advantages of the invention will be apparent from the description and the drawings, and from the claims.

Set forth below are non-limiting embodiments that are representative of the subject matter description herein:

1. An adeno-associated viral (AAV) viral genome comprising a nucleic acid sequence positioned between two inverted terminal repeats (ITRs), wherein said nucleic acid when expressed inhibits or suppresses the expression of a target gene in a cell, wherein said nucleic acid sequence comprises, in a 5′ to 3′ order: a first region encoding a first sense strand sequence, a second region encoding a first antisense strand sequence, a third region encoding a second sense strand, and a fourth region encoding a second antisense strand sequence, wherein the first and second sense strand sequences comprise at least 15 contiguous nucleotides and the first and second antisense strand sequences are complementary to an mRNA produced by the target gene and comprise at least 15 contiguous nucleotides, and wherein said first sense strand sequence and first antisense strand sequence share a region of complementarity of at least four nucleotides in length and said second sense strand sequence and second antisense strand sequence share a region of complementarity of at least four nucleotides in length.

2. An adeno-associated viral (AAV) viral genome comprising a nucleic acid sequence positioned between two inverted terminal repeats (ITRs), wherein said nucleic acid when expressed inhibits or suppresses the expression of a first target gene and a second target gene in a cell, wherein said nucleic acid sequence comprises, in a 5′ to 3′ order: a first region encoding a first sense strand sequence, a second region encoding a first antisense strand sequence, a third region encoding a second sense strand, and a fourth region encoding a second antisense strand sequence, wherein the first and second sense strand sequences comprise at least 15 contiguous nucleotides and the first antisense strand sequence is complementary to an mRNA produced by the first target gene and the second antisense strand sequence is complementary to an mRNA produced by the second target gene and comprise at least 15 contiguous nucleotides, and wherein said first sense strand sequence and first antisense strand sequence share a region of complementarity of at least four nucleotides in length and said second sense strand sequence and second antisense strand sequence share a region of complementarity of at least four nucleotides in length.

3. The AAV viral genome of embodiment 2, further comprising, in a 5′ to 3′ order, a fifth region encoding a third sense strand sequence and a sixth region encoding a third antisense strand sequence, wherein the third sense strand sequence comprises at least 15 contiguous nucleotides and the third antisense strand sequence is complementary to an mRNA produced by a third target gene and comprises at least 15 contiguous nucleotides, and wherein said third sense strand sequence and third antisense strand sequence share a region of complementarity of at least four nucleotides.

4. The AAV viral genome of embodiment 3, further comprising, in a 5′ to 3′ order, a seventh region encoding a fourth sense strand sequence and a eighth region encoding a fourth antisense strand sequence, wherein the fourth sense strand sequence comprises at least 15 contiguous nucleotides and the fourth antisense strand sequence is complementary to an mRNA produced by a fourth target gene and comprises at least 15 contiguous nucleotides, and wherein said fourth sense strand sequence and fourth antisense strand sequence share a region of complementarity of at least four nucleotides.

5. The AAV viral genome of embodiment 2, wherein the first target gene is the same as the second target gene.

6. The AAV viral genome of embodiment 3, wherein the third target gene is the same as the first target gene.

7. The AAV viral genome of embodiment 3, wherein the third target gene is the same as the second target gene.

8. The AAV viral genome of embodiment 3, wherein the first target gene, the second target gene and the third target gene are the same.

9. The AAV viral genome of embodiment 4, wherein the fourth target gene is the same as the first target gene.

10. The AAV viral genome of embodiment 4, wherein the fourth target gene is the same as the second target gene.

11. The AAV viral genome of embodiment 4, wherein the fourth target gene is the same as the third target gene.

12. The AAV viral genome of embodiment 4, wherein the fourth target gene is the same as the first target gene and the second target gene.

13. The AAV viral genome of embodiment 4, wherein the fourth target gene is the same as the second target gene and the third target gene.

14. The AAV viral genome of embodiment 4, wherein the fourth target gene is the same as the first target gene, the second target gene and the third target gene.

15. The AAV viral genome of any one of embodiments 1-14 wherein the first target gene, the second target gene, the third target gene and/or the fourth target gene is Huntingtin.

16. The AAV viral genome of any one of embodiments 1-14 wherein the first target gene, the second target gene, the third target gene and/or the fourth target gene is SOD1.

17. The AAV viral genome of any one of embodiments 1-14 wherein the first target gene, the second target gene, the third target gene and/or the fourth target gene is Huntingtin or SOD1.

18. The AAV viral genome of embodiment 1 or 2, wherein the region of complementarity between the first sense strand and the first antisense strand is at least 12 nucleotides in length.

19. The AAV viral genome of embodiment 18, wherein the region of complementarity between the first sense strand and the first antisense strand is between 14 and 21 nucleotides in length.

20. The AAV viral genome of embodiment 19, wherein the region of complementarity between the first sense strand and the first antisense strand is 19 nucleotides in length.

21. The AAV viral genome of embodiment 1 or 2, wherein the region of complementarity between the second sense strand and the second antisense strand is at least 12 nucleotides in length.

22. The AAV viral genome of embodiment 21, wherein the region of complementarity between the second sense strand and the second antisense strand is between 14 and 21 nucleotides in length.

23. The AAV viral genome of embodiment 22, wherein the region of complementarity between the second sense strand and the second antisense strand is 19 nucleotides in length.

24. The AAV viral genome of embodiment 3, wherein the region of complementarity between the third sense strand and the third antisense strand is at least 12 nucleotides in length.

25. The AAV viral genome of embodiment 24, wherein the region of complementarity between the third sense strand and the third antisense strand is between 14 and 21 nucleotides in length.

26. The AAV viral genome of embodiment 25, wherein the region of complementarity between the third sense strand and the third antisense strand is 19 nucleotides in length.

27. The AAV viral genome of embodiment 4, wherein the region of complementarity between the fourth sense strand and the fourth antisense strand is at least 12 nucleotides in length.

28. The AAV viral genome of embodiment 27, wherein the region of complementarity between the fourth sense strand and the fourth antisense strand is between 14 and 21 nucleotides in length.

29. The AAV viral genome of embodiment 25, wherein the region of complementarity between the fourth sense strand and the fourth antisense strand is 19 nucleotides in length.

30. The AAV viral genome of embodiment 1 or 2, wherein the first sense strand sequence, the second sense strand sequence, the first antisense strand sequence, and the second antisense strand sequence are, independently, 30 nucleotides or less.

31. The AAV viral genome of embodiment 3, wherein the first sense strand sequence, the second sense strand sequence, the third sense strand sequence, the first antisense strand sequence, the second antisense strand sequence and the third antisense strand sequence, are, independently, 30 nucleotides or less.

32. The AAV viral genome of embodiment 4 wherein the first sense strand sequence, the second sense strand sequence, the third sense strand sequence, the fourth sense strand sequence, the first antisense strand sequence, the second antisense strand sequence, the third antisense strand sequence and the fourth antisense strand sequence, are, independently, 30 nucleotides or less.

33. The AAV viral genome of embodiment 1 or 2, wherein at least one of the first sense strand sequence and the first antisense strand sequence or the second sense strand sequence and the second antisense strand sequence comprise a 3′ overhang of at least 1 nucleotide.

34. The AAV viral genome of embodiment 1 or 2, wherein at least one of the first sense strand sequence and the first antisense strand sequence or the second sense strand sequence and the second antisense strand sequence comprise a 3′ overhang of at least 2 nucleotides.

35. The AAV viral genome of embodiment 3, wherein the third sense strand sequence and the third antisense strand sequence comprise a 3′ overhang of at least 1 nucleotide.

36. The AAV viral genome of embodiment 3, wherein the third sense strand sequence and the third antisense strand sequence comprise a 3′ overhang of at least 2 nucleotides.

37. The AAV viral genome of embodiment 4 wherein the fourth sense strand sequence and the fourth antisense strand sequence comprise a 3′ overhang of at least 1 nucleotide.

38. The AAV viral genome of embodiment 4 wherein the fourth sense strand sequence and the fourth antisense strand sequence comprise a 3′ overhang of at least 2 nucleotides.

39. The AAV viral genome of any one of embodiments 1-38, wherein the first region comprises, a promoter 5′ of the first sense strand sequence followed by the first sense strand sequence, and the second region comprises the first antisense strand sequence followed by a promoter terminator 3′ of the first antisense strand sequence; or the third region comprises a promoter 5′ of the second sense strand sequence followed by the second sense strand sequence, and the fourth region comprises the second antisense strand sequence followed by a promoter terminator 3′ of the second antisense strand sequence.

40. The AAV viral genome of any one of embodiments 1-38, wherein the first region comprises, a promoter 5′ of the first sense strand sequence followed by the first sense strand sequence, and the second region comprises the first antisense strand sequence followed by a promoter terminator 3′ of the first antisense strand sequence; and the third region comprises a promoter 5′ of the second sense strand sequence followed by the second sense strand sequence, and the fourth region comprises the second antisense strand sequence followed by a promoter terminator 3′ of the second antisense strand sequence.

41. The AAV viral genome of any one of embodiments 3-40, wherein the fifth region comprises a promoter 5′ of the third sense strand sequence followed by the third sense strand sequence and the sixth region comprises the third antisense strand sequence followed by a promoter terminator 3′ of the third antisense strand sequence.

42. The AAV viral genome of any one of embodiments 4-41 wherein the seventh region comprises a promoter 5′ of the fourth sense strand sequence followed by the fourth sense strand sequence and the eighth region comprises the fourth antisense strand sequence followed by a promoter terminator 3′ of the fourth antisense strand sequence.

43. The AAV viral genome of embodiment 3, wherein the fifth region is 3′ of the fourth region.

44. The AAV viral genome of embodiment 4, wherein the seventh region is 3′ of the sixth region.

45. The AAV viral genome of any one of embodiments 39-44 wherein a promoter is a Pol III promoter and the promoter terminator is a Pol III promoter terminator.

46. The AAV viral genome of embodiment 45, wherein the Pol III promoter is a U3, U6, U7, 7SK, H1, or MRP, EBER, seleno-cysteine tRNA, 7SL, adenovirus VA-1, or telomerase gene promoter, and the Pol III promoter terminator is a U3, U6, U7, 7SK, H1, or MRP, EBER, seleno-cysteine tRNA, 7SL, adenovirus VA-1, or telomerase gene promoter terminator, respectively.

47. The AAV viral genome of embodiment 46, wherein the Pol III promoter is an H1 promoter and the Pol III promoter terminator is an H1 promoter terminator.

48. The AAV viral genome of any one of embodiments 1-47, wherein the AAV viral genome is a monospecific polycistronic AAV viral genome.

49 The AAV viral genome of any one of embodiments 1-47, wherein the AAV viral genome is a bispecific polycistronic AAV viral genome.

50. The AAV viral genome of embodiment 1 or 2, wherein the first region and the second region encode a first siRNA molecule, and the third region and the fourth region encode a second siRNA molecule, wherein the first and the second siRNA molecules target a different target gene.

51. The AAV viral genome of embodiment 3, wherein the fifth region and the sixth region encode a third siRNA molecule, wherein the first siRNA molecule, the second siRNA molecule and the third siRNA molecule each target a different target gene.

52. The AAV viral genome of embodiment 4, wherein the seventh region and the eighth region encode a fourth siRNA molecule, wherein the first siRNA molecule, the second siRNA molecule, the third siRNA molecule and the fourth siRNA molecule each target a different target gene.

53. An adeno-associated viral (AAV) viral genome comprising a nucleic acid sequence positioned between two inverted terminal repeats (ITRs), wherein said nucleic acid sequence comprises a first molecular scaffold region and a second molecular scaffold region, wherein said first molecular scaffold region comprises a first molecular scaffold nucleic acid sequence encoding:
(a) a first stem and loop to form a first stem-loop structure, the sequence of said first stem-loop structure from 5′ to 3′ comprising:
   i. a first UG motif at or near the base of the first 5′ stem of the first stem-loop structure;
   ii. a first 5′ stem arm comprising a first sense strand and optional first 5′ spacer region, wherein said first 5′ spacer region, when present, is located between said first UG motif and said first sense strand;
   iii. a first loop region comprising a first UGUG motif at the 5′ end of said first loop region;
   iv. a first 3′ stem arm comprising a first antisense strand and optionally a first 3′ spacer region, wherein a uridine is present at the 5′ end of said first antisense strand and wherein said first 3′ spacer region, when present, has a length sufficient to form one helical turn;
(b) a first 5′ flanking region located 5′ to said first stem-loop structure; and
(c) a first 3′ flanking region located 3′ to said first stem-loop structure, said first 3′ flanking region comprising a CNNC motif, and
   a second molecular scaffold region comprising a second molecular scaffold nucleic acid sequence encoding
(d) a second stem and loop to form a second stem-loop structure, the sequence of said second stem-loop structure from 5′ to 3′ comprising:
   v. a second UG motif at or near the base of the second 5′ stem of the second stem-loop structure;
   vi. a second 5′ stem arm comprising a second sense strand and optional second 5′ spacer region, wherein said second 5′ spacer region, when present, is located between said second UG motif and said second sense strand;
   vii. a second loop region comprising a second UGUG motif at the 5′ end of said second loop region;
   viii. a second 3′ stem arm comprising a second antisense strand and optionally a second 3′ spacer region, wherein a uridine is present at the 5′ end of said second antisense strand and wherein said second 3′ spacer region, when present, has a length sufficient to form one helical turn;
   ix. a second 5′ flanking region located 5′ to said second stem-loop structure; and
(e) a second 3′ flanking region located 3′ to said second stem-loop structure, said second 3′ flanking region comprising a CNNC motif, and
   wherein said first antisense strand and said first sense strand form a first siRNA duplex and said second antisense strand and said second sense strand form a second siRNA duplex, where the first siRNA duplex, when expressed, inhibits or suppresses the expression of a first target gene in a cell, and the second siRNA duplex, when expressed, inhibits or suppresses the expression of a second target gene in a cell, wherein the first and second sense strand sequences comprise at least 15 nucleotides, the first antisense strand sequence is complementary to an mRNA produced by the first target gene and second antisense strand sequences is complementary to an mRNA produced by the second target gene, and wherein said first sense strand sequence and first antisense strand sequence share a region of complementarity of at least four nucleotides in length and said second sense strand sequence and second antisense strand sequence share a region of complementarity of at least four nucleotides in length.

54. Adeno-associated viral (AAV) viral genome comprising a nucleic acid sequence positioned between two inverted terminal repeats (ITRs), wherein said nucleic acid sequence comprises a first molecular scaffold region and a second molecular scaffold region, wherein said first molecular scaffold region comprises a first molecular scaffold nucleic acid sequence encoding:
(a) a first stem and loop to form a first stem-loop structure, the sequence of said first stem-loop structure from 5′ to 3′ comprising:
   i. a first UG motif at or near the base of the first 5′ stem of the first stem-loop structure;
   ii. a first 5′ stem arm comprising a first antisense strand and optional first 5′ spacer region, wherein said first 5′ spacer region, when present, is located between said first UG motif and said first antisense strand;
   iii. a first loop region comprising a first UGUG motif at the 5′ end of said first loop region;
   iv. a first 3′ stem arm comprising a first sense strand and optionally a first 3′ spacer region, wherein a uridine is present at the 5′ end of said first sense strand and wherein said first 3′ spacer region, when present, has a length sufficient to form one helical turn;
(b) a first 5′ flanking region located 5′ to said first stem-loop structure; and
(c) a first 3′ flanking region located 3′ to said first stem-loop structure, said first 3′ flanking region comprising a CNNC motif, and
   a second molecular scaffold region comprising a second molecular scaffold nucleic acid sequence encoding
(d) a second stem and loop to form a second stem-loop structure, the sequence of said second stem-loop structure from 5′ to 3′ comprising:
   v. a second UG motif at or near the base of the second 5′ stem of the second stem-loop structure;
   vi. a second 5′ stem arm comprising a second antisense strand and optional second 5′ spacer region, wherein said second 5′ spacer region, when present, is located between said second UG motif and said second antisense strand;
   vii. a second loop region comprising a second UGUG motif at the 5′ end of said second loop region;
   viii. a second 3′ stem arm comprising a second sense strand and optionally a second 3′ spacer region, wherein a uridine is present at the 5′ end of said second sense strand and wherein said second 3′ spacer region, when present, has a length sufficient to form one helical turn;
(e) a second 5′ flanking region located 5′ to said second stem-loop structure; and
(f) a second 3′ flanking region located 3′ to said second stem-loop structure, said second 3′ flanking region comprising a CNNC motif, and
   wherein said first antisense strand and said first sense strand form a first siRNA duplex and said second antisense strand and said second sense strand form a second siRNA duplex, where the first siRNA duplex, when expressed, inhibits or suppresses the expression of a first target gene in a cell, and the second siRNA duplex, when expressed, inhibits or suppresses the expression of a second target gene in a cell, wherein the first and second sense strand sequences comprise at least 15 nucleotides, the first antisense strand sequence is complementary to an mRNA produced by the first target gene and second antisense strand sequences is complementary to an mRNA produced by the second target gene, and wherein said first sense strand sequence and first antisense strand sequence share a region of complementarity of at least four nucleotides in length and said second sense strand sequence and second antisense strand sequence share a region of complementarity of at least four nucleotides in length.

55. The AAV viral genome of embodiment 53 or 54, wherein the first antisense strand sequence or the second antisense strand sequence inhibits or suppresses the expression of Huntingtin.

56. The AAV viral genome of embodiment 53 or 54, wherein the first antisense strand sequence and the second antisense sequence strand inhibits or suppresses the expression of Huntingtin.

57. The AAV viral genome of embodiment 53 or 54, wherein the first antisense strand sequence or the second antisense strand sequence inhibits or suppresses the expression of SOD1.

58. The AAV viral genome of embodiment 53 or 54, wherein the first antisense strand sequence and the second antisense strand sequence inhibits or suppresses the expression of SOD1.

59. The AAV viral genome of embodiment 53 or 54, wherein the first 5′ flanking region is selected from the sequences listed in Table 10.

60. The AAV viral genome of embodiment 53 or 54, wherein the second 5′ flanking region is selected from the sequences listed in Table 10.

61. The AAV viral genome of embodiment 59, wherein the second 5′ flanking region is selected from the sequences listed in Table 10.

62. The AAV viral genome of embodiment 53 or 54, wherein the first loop region is selected from the sequences listed in Table 11.

63. The AAV viral genome of embodiment 53 or 54, wherein the second loop region is selected from the sequences listed in Table 11.

64. The AAV viral genome of embodiment 62, wherein the second loop region is selected from the sequences listed in Table 11.

65. The AAV viral genome of embodiment 53 or 54, wherein the first 3′ flanking region is selected from the sequences listed in Table 12.

66. The AAV viral genome of embodiment 53 or 54, wherein the second 3′ flanking region is selected from the sequences listed in Table 12.

67. The AAV viral genome of embodiment 65, wherein the second 3′ flanking region is selected from the sequences listed in Table 12.

68. The AAV viral genome of embodiment 53 or 54, wherein the nucleic acid sequence comprises a promoter sequence between the first molecular scaffold nucleic acid sequence and the second molecular scaffold nucleic acid sequence.

69. The AAV viral genome of embodiment 53 or 54, further comprising, in (b), a promoter 5′ of the first 5′ flanking region followed by the first 5′ flanking region and in (c) the first 3′ flanking region followed by a promoter terminator 3′ of the first '3 flanking region, and in (d), a promoter 5′ of the second 5′ flanking region followed by the second 5′ flanking region and in (e) the second 3′ flanking region followed by a promoter terminator 3′ of the second 3′ flanking region.

70. The AAV viral genome of embodiment 69, wherein the promoter is a Pol III promoter.

71. The AAV viral genome of embodiment 70, wherein the Pol III promoter sequence is a U3, U6, U7, 7SK, H1, or MRP, EBER, seleno-cysteine tRNA, 7SL, adenovirus VA-1, or telomerase gene promoter.

72. The AAV viral genome of embodiment 71, wherein the Pol III promoter is an H1 promoter.

73. The AAV viral genome of embodiment 53, wherein the nucleic acid sequence further comprises a third molecular scaffold region comprising a third molecular scaffold nucleic acid sequence encoding:
(g) a third stem and loop to form a third stem-loop structure, the sequence of said third stem-loop structure from 5′ to 3′ comprising:
   ix. a third UG motif at or near the base of the third 5′ stem of the third stem-loop structure;
   x. a third 5′ stem arm comprising a third sense strand and optional third 5′ spacer region, wherein said third 5′ spacer region, when present, is located between said third UG motif and said third sense strand;
   xi. a third loop region comprising a third UGUG motif at the 5′ end of said third loop region;
   xii. a third 3′ stem arm comprising a third antisense strand and optionally a third 3′ spacer region, wherein a uridine is present at the 5′ end of said third antisense strand and wherein said third 3′ spacer region, when present, has a length sufficient to form one helical turn;
(h) a third 5′ flanking region located 5′ to said third stem-loop structure; and
(i) a third 3′ flanking region located 3′ to said third stem-loop structure, said third 3′ flanking region comprising a CNNC motif, and
   wherein said third antisense strand and said third sense strand form a third siRNA duplex, wherein the third siRNA duplex, when expressed, inhibits or suppresses the expression of a third target gene in a cell, wherein the third sense strand sequence comprises at least 15 nucleotides, the third antisense strand sequence is complementary to an mRNA produced by the third target gene, and wherein said third sense strand sequence and third antisense strand sequence share a region of complementarity of at least four nucleotides in length.

74. The AAV viral genome of embodiment 73, further comprising, in (h), a promoter 5′ of the third 5′ flanking region followed by the third 5′ flanking region, and in (i) the third 3′ flanking region followed by a promoter terminator 3′ of the third '3 flanking region.

75. The AAV viral genome of embodiment 74, wherein the promoter is a Pol III promoter.

76. The AAV viral genome of embodiment 75, wherein the Pol III promoter sequence is a U3, U6, U7, 7SK, H1, or MRP, EBER, seleno-cysteine tRNA, 7SL, adenovirus VA-1, or telomerase gene promoter.

77. The AAV viral genome of embodiment 76, wherein the Pol III promoter is an H1 promoter.

78. The AAV viral genome of embodiment 73, wherein the nucleic acid sequence further comprises a fourth molecular scaffold region comprising a fourth molecular scaffold nucleic acid sequence encoding
(j) a fourth stem and loop to form a fourth stem-loop structure, the sequence of said fourth stem-loop structure from 5′ to 3′ comprising:
   xiii. a fourth UG motif at or near the base of the fourth 5′ stem of the fourth stem-loop structure;
   xiv. a fourth 5′ stem arm comprising a fourth sense strand and optional fourth 5′ spacer region, wherein said fourth 5′ spacer region, when present, is located between said fourth UG motif and said fourth sense strand;
   xv. a fourth loop region comprising a fourth UGUG motif at the 5′ end of said fourth loop region;
   xvi. a fourth 3′ stem arm comprising a fourth antisense strand and optionally a fourth 3′ spacer region, wherein a uridine is present at the 5′ end of said fourth antisense strand and wherein said fourth 3′ spacer region, when present, has a length sufficient to form one helical turn;
(k) a fourth 5′ flanking region located 5′ to said fourth stem-loop structure; and
(l) a fourth 3′ flanking region located 3′ to said fourth stem-loop structure, said fourth 3′ flanking region comprising a CNNC motif, and
   wherein said fourth antisense strand and said fourth sense strand form a fourth siRNA duplex, wherein the fourth siRNA duplex, when expressed, inhibits or suppresses the expression of a fourth target gene in a cell, wherein the fourth sense strand sequence comprises at least 15 nucleotides, the fourth antisense strand sequence is complementary to an mRNA produced by the fourth target gene, and wherein said fourth sense strand sequence and fourth antisense strand sequence share a region of complementarity of at least four nucleotides in length.

79. The AAV viral genome of embodiment 78, further comprising, in (k), a promoter 5′ of the fourth 5′ flanking region followed by the fourth 5′ flanking region, and in (l) the fourth 3′ flanking region followed by a promoter terminator 3′ of the fourth '3 flanking region.

80. The AAV viral genome of embodiment 79, wherein the promoter is a Pol III promoter.

81. The AAV viral genome of embodiment 80, wherein the Pol III promoter sequence is a U3, U6, U7, 7SK, H1, or MRP, EBER, seleno-cysteine tRNA, 7SL, adenovirus VA-1, or telomerase gene promoter.

82. The AAV viral genome of embodiment 81, wherein the Pol III promoter is an H1 promoter.

83. The AAV viral genome of any one of embodiments 53-82, wherein the first target gene is the same as the second target gene.

84. The AAV viral genome of any one of embodiments 53-82, wherein the third target gene is the same as the first target gene.

85. The AAV viral genome of any one of embodiments 53-82, wherein the third target gene is the same as the second target gene.

86. The AAV viral genome of any one of embodiments 53-82, wherein the first target gene, the second target gene and the third target gene are the same.

87. The AAV viral genome of any one of embodiments 53-82, wherein the fourth target gene is the same as the first target gene.

88. The AAV viral genome of any one of embodiments 53-82, wherein the fourth target gene is the same as the second target gene.

89. The AAV viral genome of any one of embodiments 53-82, wherein the fourth target gene is the same as the third target gene.

90. The AAV viral genome of any one of embodiments 53-82, wherein the fourth target gene is the same as the first target gene and the second target gene.

91. The AAV viral genome of embodiment 53-82, wherein the fourth target gene is the same as the second target gene and the third target gene.

92. The AAV viral genome of embodiment 53-82, wherein the fourth target gene is the same as the first target gene and the third target gene.

93. The AAV viral genome of any one of embodiments 53-82, wherein the fourth target gene is the same as the first target gene, the second target gene and the third target gene.

94. The AAV viral genome of any one of embodiments 53-93 wherein the first target gene, the second target gene, the third target gene and/or the fourth target gene is Huntingtin.

95. The AAV viral genome of any one of embodiments 53-93 wherein the first target gene, the second target gene, the third target gene and/or the fourth target gene is SOD1.

96. The AAV viral genome of any one of embodiments 53-93 wherein the first target gene, the second target gene, the third target gene and/or the fourth target gene is Huntingtin or SOD1.

97. A method for inhibiting the expression of a gene of a target gene in a cell comprising administering to the cell a composition comprising an AAV viral genome of any one of embodiments 1-96.

98. The method of embodiment 97, wherein the cell is a mammalian cell.

99. The method of embodiment 98, wherein the mammalian cell is a medium spiny neuron.

100. The method of embodiment 98, wherein the mammalian cell is a cortical neuron.

101. The method of embodiment 98, wherein the mammalian cell is a motor neuron.

102. The method of embodiment 98, wherein the mammalian cell is an astrocyte.

103. A method for treating a disease and/or disorder in a subject in need thereof, the method comprising administering to the subject a therapeutically effective amount of a composition comprising an AAV viral genome of any one of embodiments 1-96.

104. The method of embodiment 103, wherein the expression of a target gene is inhibited or suppressed.

105. The method of embodiment 104, wherein the expression of a target gene of interest is inhibited or suppressed by about 30% to about 70%.

106. The method of embodiment 104, wherein the expression of a target gene is inhibited or suppressed by about 50% to about 90%.

107. A method for inhibiting the expression of a target gene in a cell wherein the target gene causes a gain of function effect inside the cell, comprising administering to the cell a composition comprising an AAV viral genome of any one of embodiments 1-96.

108. The method of embodiment 107, wherein the cell is a mammalian cell.

109. The method of embodiment 108, wherein the mammalian cell is a medium spiny neuron.

110. The method of embodiment 108, wherein the mammalian cell is a cortical neuron.

111. The method of embodiment 108, wherein the mammalian cell is a motor neuron.

112. The method of embodiment 108, wherein the mammalian cell is an astrocyte.

### BRIEF DESCRIPTION OF THE DRAWINGS

The foregoing and other objects, features and advantages will be apparent from the following description of particular embodiments of the invention, as illustrated in the accompanying drawings. The drawings are not necessarily to scale, emphasis instead being placed upon illustrating the principles of various embodiments of the invention.

FIG. 1 is a schematic of a viral genome of the invention.

FIG. 2 is a schematic of a viral genome of the invention.

FIG. 3 is a schematic of a viral genome of the invention.

FIG. 4 is a schematic of a viral genome of the invention.

FIG. 5 is a schematic of a viral genome of the invention.

FIG. 6 is a schematic of a viral genome of the invention.

FIG. 7 is a schematic of a viral genome of the invention.

FIG. 8 is a schematic of a viral genome of the invention.

FIG. 9 is a schematic of a viral genome of the invention.

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

### DETAILED DESCRIPTION OF THE INVENTION

### I. Compositions of the Invention

According to the present invention, compositions for delivering modulatory polynucleotides and/or modulatory polynucleotide-based compositions by adeno-associated viruses (AAVs) are provided. AAV particles of the invention may be provided via any of several routes of administration, to a cell, tissue, organ, or organism, in vivo, ex vivo or in vitro.

As used herein, an “AAV particle” is a virus which comprises a viral genome with at least one payload region and at least one inverted terminal repeat (ITR) region.

As used herein, “viral genome” or “vector genome” or “viral vector” refers to the nucleic acid sequence(s) encapsulated in an AAV particle. Viral genomes comprise at least one payload region encoding polypeptides or fragments thereof.

As used herein, a “payload” or “payload region” is any nucleic acid molecule which encodes one or more polypeptides of the invention. At a minimum, a payload region comprises nucleic acid sequences that encode a sense and antisense sequence, an siRNA-based composition, or a fragment thereof, but may also optionally comprise one or more functional or regulatory elements to facilitate transcriptional expression and/or polypeptide translation.

The nucleic acid sequences and polypeptides disclosed herein may be engineered to contain modular elements and/or sequence motifs assembled to enable expression of the modulatory polynucleotides and/or modulatory polynucleotide-based compositions of the invention. In some embodiments, the nucleic acid sequence comprising the payload region may comprise one or more of a promoter region, an intron, a Kozak sequence, an enhancer or a polyadenylation sequence. Payload regions of the invention typically encode at least one sense and antisense sequence, an siRNA-based composition, or fragments of the foregoing in combination with each other or in combination with other polypeptide moieties.

The payload regions of the invention may be delivered to one or more target cells, tissues, organs or organisms within the viral genome of an AAV particle.

### Adeno-Associated Viruses (AAVs) and AAV Particles

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. Due to its relatively simple structure, easily manipulated using standard molecular biology techniques, this virus family is useful as a biological tool. The genome of the virus may be modified to contain a minimum of components for the assembly of a functional recombinant virus, or viral particle, which is loaded with or engineered to express or deliver a desired payload, which may be delivered to a target cell, tissue, organ, or organism.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which are incorporated by reference in their entirety.

The Parvoviridae family comprises the Dependovirus genus which includes adeno-associated viruses (AAV) capable of replication in vertebrate hosts including, but not limited to, human, primate, bovine, canine, equine, and ovine species.

The AAV viral genome is a linear, single-stranded DNA (ssDNA) molecule approximately 5,000 nucleotides (nt) in length. The AAV viral genome can comprise a payload region and at least one inverted terminal repeat (ITR) or ITR region. ITRs traditionally flank the coding nucleotide sequences for the non-structural proteins (encoded by Rep genes) and the structural proteins (encoded by capsid genes or Cap genes). While not wishing to be bound by theory, an AAV viral genome typically comprises two ITR sequences. The AAV viral genome comprises a characteristic T-shaped hairpin structure defined by the self-complementary terminal 145 nt of the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entirety.

In one embodiment, AAV particles of the present invention are recombinant AAV vectors which are replication defective, lacking sequences encoding functional Rep and Cap proteins within their viral genome. These defective AAV vectors may lack most or all parental coding sequences and essentially carry only one or two AAV ITR sequences and the nucleic acid of interest for delivery to a cell, a tissue, an organ or an organism.

In one embodiment, the viral genome of the AAV particles of the present invention comprise at least one control element which provides for the replication, transcription and translation of a coding sequence encoded therein. Not all of the control elements need always be present as long as the coding sequence is capable of being replicated, transcribed and/or translated in an appropriate host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present invention, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV vectors of the present invention may be produced recombinantly and may be based on adeno-associated virus (AAV) parent or reference sequences. As used herein, a “vector” is any molecule or moiety which transports, transduces or otherwise acts as a carrier of a heterologous molecule such as the nucleic acids described herein.

In addition to single stranded AAV viral genomes (e.g., ssAAVs), the present invention also provides for self-complementary AAV (scAAVs) viral genomes. scAAV viral genomes contain DNA strands which anneal together to form double stranded DNA. By skipping second strand synthesis, scAAVs allow for rapid expression in the cell.

In one embodiment, the AAV particle of the present invention is an scAAV.

In one embodiment, the AAV particle of the present invention is an ssAAV.

Methods for producing and/or modifying AAV particles are disclosed in the art such as pseudotyped AAV vectors (PCT Patent Publication Nos. WO200028004; WO200123001; WO2004112727; WO 2005005610 and WO 2005072364, the content of each of which is incorporated herein by reference in its entirety).

AAV particles may be modified to enhance the efficiency of delivery. Such modified AAV particles can be packaged efficiently and be used to successfully infect the target cells at high frequency and with minimal toxicity. In some embodiments the capsids of the AAV particles are engineered according to the methods described in US Publication Number US 20130195801, the contents of which are incorporated herein by reference in their entirety.

In one embodiment, the AAV particles comprising a payload region encoding the polypeptides of the invention may be introduced into mammalian cells.

### AAV Serotypes

AAV particles of the present invention may comprise or be derived from any natural or recombinant AAV serotype. According to the present invention, the AAV particles may utilize or be based on a serotype selected from any of the following AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, ovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAV-PHP.B (PHP.B), AAV-PHP.A (PHP.A), G2B-26, G2B-13, TH1.1-32, TH1.1-35, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3, AAVG2B4, AAVG2B5 and variants thereof.

In some embodiments, the AAV serotype may be, or have, a sequence as described in United States Publication No. US20030138772, the contents of which are herein incorporated by reference in their entirety, such as, but not limited to, AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAV5 (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772), AAV16.3 (US20030138772 SEQ ID NO: 10), AAV29.3/bb.1 (US20030138772 SEQ ID NO: 11), AAV29.4 (US20030138772 SEQ ID NO: 12), AAV29.5/bb.2 (US20030138772 SEQ ID NO: 13), AAV1.3 (US20030138772 SEQ ID NO: 14), AAV13.3 (US20030138772 SEQ ID NO: 15), AAV24.1 (US20030138772 SEQ ID NO: 16), AAV27.3 (US20030138772 SEQ ID NO: 17), AAV7.2 (US20030138772 SEQ ID NO: 18), AAVC1 (US20030138772 SEQ ID NO: 19), AAVC3 (US20030138772 SEQ ID NO: 20), AAVC5 (US20030138772 SEQ ID NO: 21), AAVF1 (US20030138772 SEQ ID NO: 22), AAVF3 (US20030138772 SEQ ID NO: 23), AAVF5 (US20030138772 SEQ ID NO: 24), AAVH6 (US20030138772 SEQ ID NO: 25), AAVH2 (US20030138772 SEQ ID NO: 26), AAV42-8 (US20030138772 SEQ ID NO: 27), AAV42-15 (US20030138772 SEQ ID NO: 28), AAV42-5b (US20030138772 SEQ ID NO: 29), AAV42-1b (US20030138772 SEQ ID NO: 30), AAV42-13 (US20030138772 SEQ ID NO: 31), AAV42-3a (US20030138772 SEQ ID NO: 32), AAV42-4 (US20030138772 SEQ ID NO: 33), AAV42-5a (US20030138772 SEQ ID NO: 34), AAV42-10 (US20030138772 SEQ ID NO: 35), AAV42-3b (US20030138772 SEQ ID NO: 36), AAV42-11 (US20030138772 SEQ ID NO: 37), AAV42-6b (US20030138772 SEQ ID NO: 38), AAV43-1 (US20030138772 SEQ ID NO: 39), AAV43-5 (US20030138772 SEQ ID NO: 40), AAV43-12 (US20030138772 SEQ ID NO: 41), AAV43-20 (US20030138772 SEQ ID NO: 42), AAV43-21 (US20030138772 SEQ ID NO: 43), AAV43-23 (US20030138772 SEQ ID NO: 44), AAV43-25 (US20030138772 SEQ ID NO: 45), AAV44.1 (US20030138772 SEQ ID NO: 46), AAV44.5 (US20030138772 SEQ ID NO: 47), AAV223.1 (US20030138772 SEQ ID NO: 48), AAV223.2 (US20030138772 SEQ ID NO: 49), AAV223.4 (US20030138772 SEQ ID NO: 50), AAV223.5 (US20030138772 SEQ ID NO: 51), AAV223.6 (US20030138772 SEQ ID NO: 52), AAV223.7 (US20030138772 SEQ ID NO: 53), AAVA3.4 (US20030138772 SEQ ID NO: 54), AAVA3.5 (US20030138772 SEQ ID NO: 55), AAVA3.7 (US20030138772 SEQ ID NO: 56), AAVA3.3 (US20030138772 SEQ ID NO: 57), AAV42.12 (US20030138772 SEQ ID NO: 58), AAV44.2 (US20030138772 SEQ ID NO: 59), AAV42-2 (US20030138772 SEQ ID NO: 9), or variants thereof.

In some embodiments, the AAV serotype may be, or have, a sequence as described in United States Publication No. US20150159173, the contents of which are herein incorporated by reference in their entirety, such as, but not limited to, AAV2 (SEQ ID NO: 7 and 23 of US20150159173), rh20 (SEQ ID NO: 1 of US20150159173), rh32/33 (SEQ ID NO: 2 of US20150159173), rh39 (SEQ ID NO: 3, 20 and 36 of US20150159173), rh46 (SEQ ID NO: 4 and 22 of US20150159173), rh73 (SEQ ID NO: 5 of US20150159173), rh74 (SEQ ID NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173), AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID NO: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SEQ ID NO: 47 of US20150159173), rh.58 (SEQ ID NO: 48 of US20150159173), or variants thereof including, but not limited to Cy5R1, Cy5R2, Cy5R3, Cy5R4, rh.13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In some embodiments, the AAV serotype may be, or have, a sequence as described in U.S. Pat. No. 7,198,951, the contents of which are herein incorporated by reference in their entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV8 (SEQ ID NO: 7 of U.S. Pat. No. 7,198,951).

In some embodiments, the AAV serotype may be, or have, a mutation in the AAV9 sequence as described by N Pulicherla et al. (Molecular Therapy 19(6):1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV serotype may be, or have, a sequence as described in U.S. Pat. No. 6,156,303, the contents of which are herein incorporated by reference in their entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the AAV serotype may be, or have, a sequence as described in United States Publication No. US20140359799, the contents of which are herein incorporated by reference in their entirety, such as, but not limited to, AAV8 (SEQ ID NO: 1 of US20140359799), AAVDJ (SEQ ID NO: 2 and 3 of US20140359799), or variants thereof.

In some embodiments, the serotype may be AAVDJ (AAV-DJ) or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV serotype may be, or have, a sequence of AAV4 as described in International Publication No. WO1998011244, the contents of which are herein incorporated by reference in their entirety, such as, but not limited to AAV4 (SEQ ID NO: 1-20 of WO1998011244).

In some embodiments, the AAV serotype may be, or have, a mutation in the AAV2 sequence to generate AAV2G9 as described in International Publication No. WO2014144229 and herein incorporated by reference in its entirety.

In some embodiments, the AAV serotype may be, or have, a sequence as described in International Publication No. WO2005033321, the contents of which are herein incorporated by reference in their entirety, such as, but not limited to AAV3-3 (SEQ ID NO: 217 of WO2005033321), AAV1 (SEQ ID NO: 219 and 202 of WO2005033321), AAV106.1/hu.37 (SEQ ID No: 10 of WO2005033321), AAV114.3/hu.40 (SEQ ID No: 11 of WO2005033321), AAV127.2/hu.41 (SEQ ID NO:6 and 8 of WO2005033321), AAV128.3/hu.44 (SEQ ID No: 81 of WO2005033321), AAV130.4/hu.48 (SEQ ID NO: 78 of WO2005033321), AAV145.1/hu.53 (SEQ ID No: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV16.8/hu.10 (SEQ ID NO: 156 and 56 of WO2005033321), AAV161.10/hu.60 (SEQ ID No: 170 of WO2005033321), AAV161.6/hu.61 (SEQ ID No: 174 of WO2005033321), AAV1-7/rh.48 (SEQ ID NO: 32 of WO2005033321), AAV1-8/rh.49 (SEQ ID NOs: 103 and 25 of WO2005033321), AAV2 (SEQ ID NO: 211 and 221 of WO2005033321), AAV2-15/rh.62 (SEQ ID No: 33 and 114 of WO2005033321), AAV2-3/rh.61 (SEQ ID NO: 21 of WO2005033321), AAV2-4/rh.50 (SEQ ID No: 23 and 108 of WO2005033321), AAV2-5/rh.51 (SEQ ID NO: 104 and 22 of WO2005033321), AAV3.1/hu.6 (SEQ ID NO: 5 and 84 of WO2005033321), AAV3.1/hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV33.4/hu.15 (SEQ ID No: 50 of WO2005033321), AAV33.8/hu.16 (SEQ ID No: 51 of WO2005033321), AAV3-9/rh.52 (SEQ ID NO: 96 and 18 of WO2005033321), AAV4-19/rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAV5 (SEQ ID NO: 199 and 216 of WO2005033321), AAV52.1/hu.20 (SEQ ID NO: 63 of WO2005033321), AAV52/hu.19 (SEQ ID NO: 133 of WO2005033321), AAV5-22/rh.58 (SEQ ID No: 27 of WO2005033321), AAV5-3/rh.57 (SEQ ID NO: 105 of WO2005033321), AAV5-3/rh.57 (SEQ ID No: 26 of WO2005033321), AAV58.2/hu.25 (SEQ ID No: 49 of WO2005033321), AAV6 (SEQ ID NO: 203 and 220 of WO2005033321), AAV7 (SEQ ID NO: 222 and 213 of WO2005033321), AAV7.3/hu.7 (SEQ ID No: 55 of WO2005033321), AAV8 (SEQ ID NO: 223 and 214 of WO2005033321), AAVH-1/hu.1 (SEQ ID No: 46 of WO2005033321), AAVH-5/hu.3 (SEQ ID No: 44 of WO2005033321), AAVhu.1 (SEQ ID NO: 144 of WO2005033321), AAVhu.10 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu.14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ ID NO: 149 of WO2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ ID NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ ID NO: 164 of WO2005033321), AAVhu.37 (SEQ ID NO: 88 of WO2005033321), AAVhu.39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ ID NO: 160 of WO2005033321), AAVhu.44 (SEQ ID NO: 144 of WO2005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ ID NO: 189 of WO2005033321), AAVhu.51 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61 (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ ID NO: 150 of WO2005033321), AAVhu.8 (WO2005033321 SEQ ID NO: 12), AAVhu.9 (SEQ ID NO: 155 of WO2005033321), AAVLG-10/rh.40 (SEQ ID No: 14 of WO2005033321), AAVLG-4/rh.38 (SEQ ID NO: 86 of WO2005033321), AAVLG-4/rh.38 (SEQ ID No: 7 of WO2005033321), AAVN721-8/rh.43 (SEQ ID NO: 163 of WO2005033321), AAVN721-8/rh.43 (SEQ ID No: 43 of WO2005033321), AAVpi.1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ ID NO: 38), AAVrh.48 (SEQ ID NO: 115 of WO2005033321), AAVrh.49 (SEQ ID NO: 103 of WO2005033321), AAVrh.50 (SEQ ID NO: 108 of WO2005033321), AAVrh.51 (SEQ ID NO: 104 of WO2005033321), AAVrh.52 (SEQ ID NO: 96 of WO2005033321), AAVrh.53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ ID NO: 106 of WO2005033321), AAVrh.59 (WO2005033321 SEQ ID NO: 42), AAVrh.60 (WO2005033321 SEQ ID NO: 31), AAVrh.61 (SEQ ID NO: 107 of WO2005033321), AAVrh.62 (SEQ ID NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9), or variants thereof including, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25, AAVrh.25/42 15, AAVrh.31, AAVrh.32, AAVrh.33, AAVrh.34, AAVrh.35, AAVrh.36, AAVrh.37, AAVrh14. Non limiting examples of variants include SEQ ID NO: 13, 15, 17, 19, 24, 36, 40, 45, 47, 48, 51-54, 60-62, 64-77, 79, 80, 82, 89, 90, 93-95, 98, 100, 101, 109-113, 118-120, 124, 126, 131, 139, 142, 151,154, 158, 161, 162, 165-183, 202, 204-212, 215, 219, 224-236, of WO2005033321, the contents of which are herein incorporated by reference in their entirety.

In some embodiments, the AAV serotype may be, or have, a sequence as described in International Publication No. WO2015168666, the contents of which are herein incorporated by reference in their entirety, such as, but not limited to, AAVrh8R (SEQ ID NO: 9 of WO2015168666), AAVrh8R A586R mutant (SEQ ID NO: 10 of WO2015168666), AAVrh8R R533A mutant (SEQ ID NO: 11 of WO2015168666), or variants thereof.

In some embodiments, the AAV serotype may be, or have, a sequence as described in U.S. Pat. No. 9,233,131, the contents of which are herein incorporated by reference in their entirety, such as, but not limited to, AAVhE1.1 (SEQ ID NO:44 of U.S. Pat. No. 9,233,131), AAVhEr1.5 (SEQ ID NO:45 of U.S. Pat. No. 9,233,131), AAVhER1.14 (SEQ ID NO:46 of U.S. Pat. No. 9,233,131), AAVhEr1.8 (SEQ ID NO:47 of U.S. Pat. No. 9,233,131), AAVhEr1.16 (SEQ ID NO:48 of U.S. Pat. No. 9,233,131), AAVhEr1.18 (SEQ ID NO:49 of U.S. Pat. No. 9,233,131), AAVhEr1.35 (SEQ ID NO:50 of U.S. Pat. No. 9,233,131), AAVhEr1.7 (SEQ ID NO:51 of U.S. Pat. No. 9,233,131), AAVhEr1.36 (SEQ ID NO:52 of U.S. Pat. No. 9,233,131), AAVhEr2.29 (SEQ ID NO:53 of U.S. Pat. No. 9,233,131), AAVhEr2.4 (SEQ ID NO:54 of U.S. Pat. No. 9,233,131), AAVhEr2.16 (SEQ ID NO:55 of U.S. Pat. No. 9,233,131), AAVhEr2.30 (SEQ ID NO:56 of U.S. Pat. No. 9,233,131), AAVhEr2.31 (SEQ ID NO:58 of U.S. Pat. No. 9,233,131), AAVhEr2.36 (SEQ ID NO:57 of U.S. Pat. No. 9,233,131), AAVhER1.23 (SEQ ID NO:53 of U.S. Pat. No. 9,233,131), AAVhEr3.1 (SEQ ID NO:59 of U.S. Pat. No. 9,233,131), AAV2.5T (SEQ ID NO:42 of U.S. Pat. No. 9,233,131), or variants thereof.

In some embodiments, the AAV serotype may be, or have, a sequence as described in United States Patent Publication No. US20150376607, the contents of which are herein incorporated by reference in their entirety, such as, but not limited to, AAV-PAEC (SEQ ID NO:1 of US20150376607), AAV-LK01 (SEQ ID NO:2 of US20150376607), AAV-LK02 (SEQ ID NO:3 of US20150376607), AAV-LK03 (SEQ ID NO:4 of US20150376607), AAV-LK04 (SEQ ID NO:5 of US20150376607), AAV-LK05 (SEQ ID NO:6 of US20150376607), AAV-LK06 (SEQ ID NO:7 of US20150376607), AAV-LK07 (SEQ ID NO:8 of US20150376607), AAV-LK08 (SEQ ID NO:9 of US20150376607), AAV-LK09 (SEQ ID NO:10 of US20150376607), AAV-LK10 (SEQ ID NO:11 of US20150376607), AAV-LK11 (SEQ ID NO:12 of US20150376607), AAV-LK12 (SEQ ID NO:13 of US20150376607), AAV-LK13 (SEQ ID NO:14 of US20150376607), AAV-LK14 (SEQ ID NO:15 of US20150376607), AAV-LK15 (SEQ ID NO:16 of US20150376607), AAV-LK16 (SEQ ID NO:17 of US20150376607), AAV-LK17 (SEQ ID NO:18 of US20150376607), AAV-LK18 (SEQ ID NO:19 of US20150376607), AAV-LK19 (SEQ ID NO:20 of US20150376607), AAV-PAEC2 (SEQ ID NO:21 of US20150376607), AAV-PAEC4 (SEQ ID NO:22 of US20150376607), AAV-PAEC6 (SEQ ID NO:23 of US20150376607), AAV-PAEC7 (SEQ ID NO:24 of US20150376607), AAV-PAEC8 (SEQ ID NO:25 of US20150376607), AAV-PAEC11 (SEQ ID NO:26 of US20150376607), AAV-PAEC12 (SEQ ID NO:27, of US20150376607), or variants thereof.

In some embodiments, the AAV serotype may be, or have, a sequence as described in U.S. Pat. No. 9,163,261, the contents of which are herein incorporated by reference in their entirety, such as, but not limited to, AAV-2-pre-miRNA-101 (SEQ ID NO: 1 U.S. Pat. No. 9,163,261), or variants thereof.

In some embodiments, the AAV serotype may be, or have, a sequence as described in United States Patent Publication No. US20150376240, the contents of which are herein incorporated by reference in their entirety, such as, but not limited to, AAV-8h (SEQ ID NO: 6 of US20150376240), AAV-8b (SEQ ID NO: 5 of US20150376240), AAV-h (SEQ ID NO: 2 of US20150376240), AAV-b (SEQ ID NO: 1 of US20150376240), or variants thereof.

In some embodiments, the AAV serotype may be, or have, a sequence as described in United States Patent Publication No. US20160017295, the contents of which are herein incorporated by reference in their entirety, such as, but not limited to, AAV SM 10-2 (SEQ ID NO: 22 of US20160017295), AAV Shuffle 100-1 (SEQ ID NO: 23 of US20160017295), AAV Shuffle 100-3 (SEQ ID NO: 24 of US20160017295), AAV Shuffle 100-7 (SEQ ID NO: 25 of US20160017295), AAV Shuffle 10-2 (SEQ ID NO: 34 of US20160017295), AAV Shuffle 10-6 (SEQ ID NO: 35 of US20160017295), AAV Shuffle 10-8 (SEQ ID NO: 36 of US20160017295), AAV Shuffle 100-2 (SEQ ID NO: 37 of US20160017295), AAV SM 10-1 (SEQ ID NO: 38 of US20160017295), AAV SM 10-8 (SEQ ID NO: 39 of US20160017295), AAV SM 100-3 (SEQ ID NO: 40 of US20160017295), AAV SM 100-10 (SEQ ID NO: 41 of US20160017295), or variants thereof.

In some embodiments, the AAV serotype may be, or have, a sequence as described in United States Patent Publication No. US20150238550, the contents of which are herein incorporated by reference in their entirety, such as, but not limited to, BNP61 AAV (SEQ ID NO: 1 of US20150238550), BNP62 AAV (SEQ ID NO: 3 of US20150238550), BNP63 AAV (SEQ ID NO: 4 of US20150238550), or variants thereof.

In some embodiments, the AAV serotype may be or may have a sequence as described in United States Patent Publication No. US20150315612, the contents of which are herein incorporated by reference in their entirety, such as, but not limited to, AAVrh.50 (SEQ ID NO: 108 of US20150315612), AAVrh.43 (SEQ ID NO: 163 of US20150315612), AAVrh.62 (SEQ ID NO: 114 of US20150315612), AAVrh.48 (SEQ ID NO: 115 of US20150315612), AAVhu.19 (SEQ ID NO: 133 of US20150315612), AAVhu.11 (SEQ ID NO: 153 of US20150315612), AAVhu.53 (SEQ ID NO: 186 of US20150315612), AAV4-8/rh.64 (SEQ ID No: 15 of US20150315612), AAVLG-9/hu.39 (SEQ ID No: 24 of US20150315612), AAV54.5/hu.23 (SEQ ID No: 60 of US20150315612), AAV54.2/hu.22 (SEQ ID No: 67 of US20150315612), AAV54.7/hu.24 (SEQ ID No: 66 of US20150315612), AAV54.1/hu.21 (SEQ ID No: 65 of US20150315612), AAV54.4R/hu.27 (SEQ ID No: 64 of US20150315612), AAV46.2/hu.28 (SEQ ID No: 68 of US20150315612), AAV46.6/hu.29 (SEQ ID No: 69 of US20150315612), AAV128.1/hu.43 (SEQ ID No: 80 of US20150315612), or variants thereof.

In some embodiments, the AAV serotype may be, or have, a sequence as described in International Publication No. WO2015121501, the contents of which are herein incorporated by reference in their entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

According to the present invention, AAV capsid serotype selection or use may be from a variety of species. In one embodiment, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In one embodiment, the AAV may be a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In one embodiment, the AAV may be a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of U.S. Pat. No. 7,427,396), or variants thereof.

In other embodiments the AAV may be engineered as a hybrid AAV from two or more parental serotypes. In one embodiment, the AAV may be AAV2G9 which comprises sequences from AAV2 and AAV9. The AAV2G9 AAV serotype may be, or have, a sequence as described in United States Patent Publication No. US20160017005, the contents of which are herein incorporated by reference in its entirety.

In one embodiment, the AAV may be a serotype generated by the AAV9 capsid library with mutations in amino acids 390-627 (VP1 numbering) as described by Pulicherla et al. (Molecular Therapy 19(6):1070-1078 (2011), the contents of which are herein incorporated by reference in their entirety. The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In some embodiments, the AAV serotype may be, or have, a sequence as described in International Publication No. WO2016049230, the contents of which are herein incorporated by reference in their entirety, such as, but not limited to AAVF1/HSC1 (SEQ ID NO: 2 and 20 of WO2016049230), AAVF2/HSC2 (SEQ ID NO: 3 and 21 of WO2016049230), AAVF3/HSC3 (SEQ ID NO: 5 and 22 of WO2016049230), AAVF4/HSC4 (SEQ ID NO: 6 and 23 of WO2016049230), AAVF5/HSC5 (SEQ ID NO: 11 and 25 of WO2016049230), AAVF6/HSC6 (SEQ ID NO: 7 and 24 of WO2016049230), AAVF7/HSC7 (SEQ ID NO: 8 and 27 of WO2016049230), AAVF8/HSC8 (SEQ ID NO: 9 and 28 of WO2016049230), AAVF9/HSC9 (SEQ ID NO: 10 and 29 of WO2016049230), AAVF11/HSC11 (SEQ ID NO: 4 and 26 of WO2016049230), AAVF12/HSC12 (SEQ ID NO: 12 and 30 of WO2016049230), AAVF13/HSC13 (SEQ ID NO: 14 and 31 of WO2016049230), AAVF14/HSC14 (SEQ ID NO: 15 and 32 of WO2016049230), AAVF15/HSC15 (SEQ ID NO: 16 and 33 of WO2016049230), AAVF16/HSC16 (SEQ ID NO: 17 and 34 of WO2016049230), AAVF17/HSC17 (SEQ ID NO: 13 and 35 of WO2016049230), or variants or derivatives thereof.

In some embodiments, the AAV serotype may be, or have, a sequence as described in U.S. Pat. No. 8,734,809, the contents of which are herein incorporated by reference in their entirety, such as, but not limited to, AAV CBr-E1 (SEQ ID NO: 13 and 87 of U.S. Pat. No. 8,734,809), AAV CBr-E2 (SEQ ID NO: 14 and 88 of U.S. Pat. No. 8,734,809), AAV CBr-E3 (SEQ ID NO: 15 and 89 of U.S. Pat. No. 8,734,809), AAV CBr-E4 (SEQ ID NO: 16 and 90 of U.S. Pat. No. 8,734,809), AAV CBr-E5 (SEQ ID NO: 17 and 91 of U.S. Pat. No. 8,734,809), AAV CBr-e5 (SEQ ID NO: 18 and 92 of U.S. Pat. No. 8,734,809), AAV CBr-E6 (SEQ ID NO: 19 and 93 of U.S. Pat. No. 8,734,809), AAV CBr-E7 (SEQ ID NO: 20 and 94 of U.S. Pat. No. 8,734,809), AAV CBr-E8 (SEQ ID NO: 21 and 95 of U.S. Pat. No. 8,734,809), AAV CLv-D1 (SEQ ID NO: 22 and 96 of U.S. Pat. No. 8,734,809), AAV CLv-D2 (SEQ ID NO: 23 and 97 of U.S. Pat. No. 8,734,809), AAV CLv-D3 (SEQ ID NO: 24 and 98 of U.S. Pat. No. 8,734,809), AAV CLv-D4 (SEQ ID NO: 25 and 99 of U.S. Pat. No. 8,734,809), AAV CLv-D5 (SEQ ID NO: 26 and 100 of U.S. Pat. No. 8,734,809), AAV CLv-D6 (SEQ ID NO: 27 and 101 of U.S. Pat. No. 8,734,809), AAV CLv-D7 (SEQ ID NO: 28 and 102 of U.S. Pat. No. 8,734,809), AAV CLv-D8 (SEQ ID NO: 29 and 103 of U.S. Pat. No. 8,734,809), AAV CLv-E1 (SEQ ID NO: 13 and 87 of U.S. Pat. No. 8,734,809), AAV CLv-R1 (SEQ ID NO: 30 and 104 of U.S. Pat. No. 8,734,809), AAV CLv-R2 (SEQ ID NO: 31 and 105 of U.S. Pat. No. 8,734,809), AAV CLv-R3 (SEQ ID NO: 32 and 106 of U.S. Pat. No. 8,734,809), AAV CLv-R4 (SEQ ID NO: 33 and 107 of U.S. Pat. No. 8,734,809), AAV CLv-R5 (SEQ ID NO: 34 and 108 of U.S. Pat. No. 8,734,809), AAV CLv-R6 (SEQ ID NO: 35 and 109 of U.S. Pat. No. 8,734,809), AAV CLv-R7 (SEQ ID NO: 36 and 110 of U.S. Pat. No. 8,734,809), AAV CLv-R8 (SEQ ID NO: 37 and 111 of U.S. Pat. No. 8,734,809), AAV CLv-R9 (SEQ ID NO: 38 and 112 of U.S. Pat. No. 8,734,809), AAV CLg-F1 (SEQ ID NO: 39 and 113 of U.S. Pat. No. 8,734,809), AAV CLg-F2 (SEQ ID NO: 40 and 114 of U.S. Pat. No. 8,734,809), AAV CLg-F3 (SEQ ID NO: 41 and 115 of U.S. Pat. No. 8,734,809), AAV CLg-F4 (SEQ ID NO: 42 and 116 of U.S. Pat. No. 8,734,809), AAV CLg-F5 (SEQ ID NO: 43 and 117 of U.S. Pat. No. 8,734,809), AAV CLg-F6 (SEQ ID NO: 43 and 117 of U.S. Pat. No. 8,734,809), AAV CLg-F7 (SEQ ID NO: 44 and 118 of U.S. Pat. No. 8,734,809), AAV CLg-F8 (SEQ ID NO: 43 and 117 of U.S. Pat. No. 8,734,809), AAV CSp-1 (SEQ ID NO: 45 and 119 of U.S. Pat. No. 8,734,809), AAV CSp-10 (SEQ ID NO: 46 and 120 of U.S. Pat. No. 8,734,809), AAV CSp-11 (SEQ ID NO: 47 and 121 of U.S. Pat. No. 8,734,809), AAV CSp-2 (SEQ ID NO: 48 and 122 of U.S. Pat. No. 8,734,809), AAV CSp-3 (SEQ ID NO: 49 and 123 of U.S. Pat. No. 8,734,809), AAV CSp-4 (SEQ ID NO: 50 and 124 of U.S. Pat. No. 8,734,809), AAV CSp-6 (SEQ ID NO: 51 and 125 of U.S. Pat. No. 8,734,809), AAV CSp-7 (SEQ ID NO: 52 and 126 of U.S. Pat. No. 8,734,809), AAV CSp-8 (SEQ ID NO: 53 and 127 of U.S. Pat. No. 8,734,809), AAV CSp-9 (SEQ ID NO: 54 and 128 of U.S. Pat. No. 8,734,809), AAV CHt-2 (SEQ ID NO: 55 and 129 of U.S. Pat. No. 8,734,809), AAV CHt-3 (SEQ ID NO: 56 and 130 of U.S. Pat. No. 8,734,809), AAV CKd-1 (SEQ ID NO: 57 and 131 of U.S. Pat. No. 8,734,809), AAV CKd-10 (SEQ ID NO: 58 and 132 of U.S. Pat. No. 8,734,809), AAV CKd-2 (SEQ ID NO: 59 and 133 of U.S. Pat. No. 8,734,809), AAV CKd-3 (SEQ ID NO: 60 and 134 of U.S. Pat. No. 8,734,809), AAV CKd-4 (SEQ ID NO: 61 and 135 of U.S. Pat. No. 8,734,809), AAV CKd-6 (SEQ ID NO: 62 and 136 of U.S. Pat. No. 8,734,809), AAV CKd-7 (SEQ ID NO: 63 and 137 of U.S. Pat. No. 8,734,809), AAV CKd-8 (SEQ ID NO: 64 and 138 of U.S. Pat. No. 8,734,809), AAV CLv-1 (SEQ ID NO: 35 and 139 of U.S. Pat. No. 8,734,809), AAV CLv-12 (SEQ ID NO: 66 and 140 of U.S. Pat. No. 8,734,809), AAV CLv-13 (SEQ ID NO: 67 and 141 of U.S. Pat. No. 8,734,809), AAV CLv-2 (SEQ ID NO: 68 and 142 of U.S. Pat. No. 8,734,809), AAV CLv-3 (SEQ ID NO: 69 and 143 of U.S. Pat. No. 8,734,809), AAV CLv-4 (SEQ ID NO: 70 and 144 of U.S. Pat. No. 8,734,809), AAV CLv-6 (SEQ ID NO: 71 and 145 of U.S. Pat. No. 8,734,809), AAV CLv-8 (SEQ ID NO: 72 and 146 of U.S. Pat. No. 8,734,809), AAV CKd-B1 (SEQ ID NO: 73 and 147 of U.S. Pat. No. 8,734,809), AAV CKd-B2 (SEQ ID NO: 74 and 148 of U.S. Pat. No. 8,734,809), AAV CKd-B3 (SEQ ID NO: 75 and 149 of U.S. Pat. No. 8,734,809), AAV CKd-B4 (SEQ ID NO: 76 and 150 of U.S. Pat. No. 8,734,809), AAV CKd-B5 (SEQ ID NO: 77 and 151 of U.S. Pat. No. 8,734,809), AAV CKd-B6 (SEQ ID NO: 78 and 152 of U.S. Pat. No. 8,734,809), AAV CKd-B7 (SEQ ID NO: 79 and 153 of U.S. Pat. No. 8,734,809), AAV CKd-B8 (SEQ ID NO: 80 and 154 of U.S. Pat. No. 8,734,809), AAV CKd-H1 (SEQ ID NO: 81 and 155 of U.S. Pat. No. 8,734,809), AAV CKd-H2 (SEQ ID NO: 82 and 156 of U.S. Pat. No. 8,734,809), AAV CKd-H3 (SEQ ID NO: 83 and 157 of U.S. Pat. No. 8,734,809), AAV CKd-H4 (SEQ ID NO: 84 and 158 of U.S. Pat. No. 8,734,809), AAV CKd-H5 (SEQ ID NO: 85 and 159 of U.S. Pat. No. 8,734,809), AAV CKd-H6 (SEQ ID NO: 77 and 151 of U.S. Pat. No. 8,734,809), AAV CHt-1 (SEQ ID NO: 86 and 160 of U.S. Pat. No. 8,734,809), AAV CLv1-1 (SEQ ID NO: 171 of U.S. Pat. No. 8,734,809), AAV CLv1-2 (SEQ ID NO: 172 of U.S. Pat. No. 8,734,809), AAV CLv1-3 (SEQ ID NO: 173 of U.S. Pat. No. 8,734,809), AAV CLv1-4 (SEQ ID NO: 174 of U.S. Pat. No. 8,734,809), AAV Clv1-7 (SEQ ID NO: 175 of U.S. Pat. No. 8,734,809), AAV Clv1-8 (SEQ ID NO: 176 of U.S. Pat. No. 8,734,809), AAV Clv1-9 (SEQ ID NO: 177 of U.S. Pat. No. 8,734,809), AAV Clv1-10 (SEQ ID NO: 178 of U.S. Pat. No. 8,734,809), AAV.VR-355 (SEQ ID NO: 181 of U.S. Pat. No. 8,734,809), AAV.hu.48R3 (SEQ ID NO: 183 of U.S. Pat. No. 8,734,809), or variants or derivatives thereof.

In some embodiments, the AAV serotype may be, or have, a sequence as described in International Publication No. WO2016065001, the contents of which are herein incorporated by reference in their entirety, such as, but not limited to AAV CHt-P2 (SEQ ID NO: 1 and 51 of WO2016065001), AAV CHt-P5 (SEQ ID NO: 2 and 52 of WO2016065001), AAV CHt-P9 (SEQ ID NO: 3 and 53 of WO2016065001), AAV CBr-7.1 (SEQ ID NO: 4 and 54 of WO2016065001), AAV CBr-7.2 (SEQ ID NO: 5 and 55 of WO2016065001), AAV CBr-7.3 (SEQ ID NO: 6 and 56 of WO2016065001), AAV CBr-7.4 (SEQ ID NO: 7 and 57 of WO2016065001), AAV CBr-7.5 (SEQ ID NO: 8 and 58 of WO2016065001), AAV CBr-7.7 (SEQ ID NO: 9 and 59 of WO2016065001), AAV CBr-7.8 (SEQ ID NO: 10 and 60 of WO2016065001), AAV CBr-7.10 (SEQ ID NO: 11 and 61 of WO2016065001), AAV CKd-N3 (SEQ ID NO: 12 and 62 of WO2016065001), AAV CKd-N4 (SEQ ID NO: 13 and 63 of WO2016065001), AAV CKd-N9 (SEQ ID NO: 14 and 64 of WO2016065001), AAV CLv-L4 (SEQ ID NO: 15 and 65 of WO2016065001), AAV CLv-L5 (SEQ ID NO: 16 and 66 of WO2016065001), AAV CLv-L6 (SEQ ID NO: 17 and 67 of WO2016065001), AAV CLv-K1 (SEQ ID NO: 18 and 68 of WO2016065001), AAV CLv-K3 (SEQ ID NO: 19 and 69 of WO2016065001), AAV CLv-K6 (SEQ ID NO: 20 and 70 of WO2016065001), AAV CLv-M1 (SEQ ID NO: 21 and 71 of WO2016065001), AAV CLv-M11 (SEQ ID NO: 22 and 72 of WO2016065001), AAV CLv-M2 (SEQ ID NO: 23 and 73 of WO2016065001), AAV CLv-M5 (SEQ ID NO: 24 and 74 of WO2016065001), AAV CLv-M6 (SEQ ID NO: 25 and 75 of WO2016065001), AAV CLv-M7 (SEQ ID NO: 26 and 76 of WO2016065001), AAV CLv-M8 (SEQ ID NO: 27 and 77 of WO2016065001), AAV CLv-M9 (SEQ ID NO: 28 and 78 of WO2016065001), AAV CHt-P1 (SEQ ID NO: 29 and 79 of WO2016065001), AAV CHt-P6 (SEQ ID NO: 30 and 80 of WO2016065001), AAV CHt-P8 (SEQ ID NO: 31 and 81 of WO2016065001), AAV CHt-6.1 (SEQ ID NO: 32 and 82 of WO2016065001), AAV CHt-6.10 (SEQ ID NO: 33 and 83 of WO2016065001), AAV CHt-6.5 (SEQ ID NO: 34 and 84 of WO2016065001), AAV CHt-6.6 (SEQ ID NO: 35 and 85 of WO2016065001), AAV CHt-6.7 (SEQ ID NO: 36 and 86 of WO2016065001), AAV CHt-6.8 (SEQ ID NO: 37 and 87 of WO2016065001), AAV CSp-8.10 (SEQ ID NO: 38 and 88 of WO2016065001), AAV CSp-8.2 (SEQ ID NO: 39 and 89 of WO2016065001), AAV CSp-8.4 (SEQ ID NO: 40 and 90 of WO2016065001), AAV CSp-8.5 (SEQ ID NO: 41 and 91 of WO2016065001), AAV CSp-8.6 (SEQ ID NO: 42 and 92 of WO2016065001), AAV CSp-8.7 (SEQ ID NO: 43 and 93 of WO2016065001), AAV CSp-8.8 (SEQ ID NO: 44 and 94 of WO2016065001), AAV CSp-8.9 (SEQ ID NO: 45 and 95 of WO2016065001), AAV CBr-B7.3 (SEQ ID NO: 46 and 96 of WO2016065001), AAV CBr-B7.4 (SEQ ID NO: 47 and 97 of WO2016065001), AAV3B (SEQ ID NO: 48 and 98 of WO2016065001), AAV4 (SEQ ID NO: 49 and 99 of WO2016065001), AAV5 (SEQ ID NO: 50 and 100 of WO2016065001), or variants or derivatives thereof.

In some embodiments, the AAV serotype may be, or have, a modification as described in United States Publication No. US 20160361439, the contents of which are herein incorporated by reference in their entirety, such as but not limited to, Y252F, Y272F, Y444F, Y500F, Y700F, Y704F, Y730F, Y275F, Y281F, Y508F, Y576F, Y612G, Y673F, and Y720F of the wild-type AAV1, AAV2, AAV3, AAV4, AAV5, AAV6, AAV7, AAV8, AAV9, AAV10, AAV11, AAV12, and hybrids thereof.

In some embodiments, the AAV serotype may be, or have, a mutation as described in U.S. Pat. No. 9,546,112, the contents of which are herein incorporated by reference in their entirety, such as, but not limited to, at least two, but not all the F129L, D418E, K531E, L584F, V598A and H642N mutations in the sequence of AAV6 (SEQ ID NO:4 of U.S. Pat. No. 9,546,112), AAV1 (SEQ ID NO:6 of U.S. Pat. No. 9,546,112), AAV2, AAV3, AAV4, AAV5, AAV7, AAV9, AAV10 or AAV11 or derivatives thereof. In yet another embodiment, the AAV serotype may be, or have, an AAV6 sequence comprising the K531E mutation (SEQ ID NO:5 of U.S. Pat. No. 9,546,112).

In some embodiments, the AAV serotype may be, or have, a mutation in the AAV1 sequence, as described in in United States Publication No. US 20130224836, the contents of which are herein incorporated by reference in their entirety, such as, but not limited to, at least one of the surface-exposed tyrosine residues, preferably, at positions 252, 273, 445, 701, 705 and 731 of AAV1 (SEQ ID NO: 2 of US 20130224836) substituted with another amino acid, preferably with a phenylalanine residue. In one embodiment, the AAV serotype may be, or have, a mutation in the AAV9 sequence, such as, but not limited to, at least one of the surface-exposed tyrosine residues, preferably, at positions 252, 272, 444, 500, 700, 704 and 730 of AAV2 (SEQ ID NO: 4 of US 20130224836) substituted with another amino acid, preferably with a phenylalanine residue. In one embodiment, the tyrosine residue at position 446 of AAV9 (SEQ ID NO: 6 US 20130224836) is substituted with a phenylalanine residue.

In some embodiments, the serotype may be AAV2 or a variant thereof, as described in International Publication No. WO2016130589, herein incorporated by reference in its entirety. The amino acid sequence of AAV2 may comprise N587A, E548A, or N708A mutations. In one embodiment, the amino acid sequence of any AAV may comprise a V708K mutation.

In one embodiment, the AAV may be a serotype selected from any of those found in Table 1.

In one embodiment, the AAV may comprise a sequence, fragment or variant thereof, of the sequences in Table 1.

In one embodiment, the AAV may be encoded by a sequence, fragment or variant as described in Table 1.

TABLE 1
AAV Serotypes

| Serotype | SEQ ID NO | Reference Information |
|---|---|---|
| AAV1 | 1 | US20150159173 SEQ ID NO: 11, |
| | | US20150315612 SEQ ID NO: 202 |
| AAV1 | 2 | US20160017295 SEQ ID NO: |
| | | 1US20030138772 SEQ ID NO: 64, |
| | | US20150159173 SEQ ID NO: 27, |
| | | US20150315612 SEQ ID NO: 219, |
| | | U.S. Pat. No. 7,198,951 SEQ ID NO: 5 |
| AAV1 | 3 | US20030138772 SEQ ID NO: 6 |
| AAV1.3 | 4 | US20030138772 SEQ ID NO: 14 |
| AAV10 | 5 | US20030138772 SEQ ID NO: 117 |
| AAV10 | 6 | WO2015121501 SEQ ID NO: 9 |
| AAV10 | 7 | WO2015121501 SEQ ID NO: 8 |
| AAV11 | 8 | US20030138772 SEQ ID NO: 118 |
| AAV12 | 9 | US20030138772 SEQ ID NO: 119 |
| AAV2 | 10 | US20150159173 SEQ ID NO: 7, |
| | | US20150315612 SEQ ID NO: 211 |
| AAV2 | 11 | US20030138772 SEQ ID NO: 70, |
| | | US20150159173 SEQ ID NO: 23, |
| | | US20150315612 SEQ ID NO: 221, |
| | | US20160017295 SEQ ID NO: 2, |
| | | U.S. Pat. No. 6,156,303 SEQ ID NO: 4, |
| | | U.S. Pat. No. 7,198,951 SEQ ID NO: 4, |
| | | WO2015121501 SEQ ID NO: 1 |
| AAV2 | 12 | U.S. Pat. No. 6,156,303 SEQ ID NO: 8 |
| AAV2 | 13 | US20030138772 SEQ ID NO: 7 |
| AAV2 | 14 | U.S. Pat. No. 6,156,303 SEQ ID NO: 3 |
| AAV2.5T | 15 | U.S. Pat. No. 9,233,131 SEQ ID NO: 42 |
| AAV223.10 | 16 | US20030138772 SEQ ID NO: 75 |
| AAV223.2 | 17 | US20030138772 SEQ ID NO: 49 |
| AAV223.2 | 18 | US20030138772 SEQ ID NO: 76 |
| AAV223.4 | 19 | US20030138772 SEQ ID NO: 50 |
| AAV223.4 | 20 | US20030138772 SEQ ID NO: 73 |
| AAV223.5 | 21 | US20030138772 SEQ ID NO: 51 |
| AAV223.5 | 22 | US20030138772 SEQ ID NO: 74 |
| AAV223.6 | 23 | US20030138772 SEQ ID NO: 52 |
| AAV223.6 | 24 | US20030138772 SEQ ID NO: 78 |
| AAV223.7 | 25 | US20030138772 SEQ ID NO: 53 |
| AAV223.7 | 26 | US20030138772 SEQ ID NO: 77 |
| AAV29.3 | 27 | US20030138772 SEQ ID NO: 82 |
| AAV29.4 | 28 | US20030138772 SEQ ID NO: 12 |
| AAV29.5 | 29 | US20030138772 SEQ ID NO: 83 |
| AAV29.5 | 30 | US20030138772 SEQ ID NO: 13 |
| (AAVbb.2) | | |
| AAV3 | 31 | US20150159173 SEQ ID NO: 12 |
| AAV3 | 32 | US20030138772 SEQ ID NO: 71, |
| | | US20150159173 SEQ ID NO: 28, |
| | | US20160017295 SEQ ID NO: 3, |
| | | U.S. Pat. No. 7,198,951 SEQ ID NO: 6 |
| AAV3 | 33 | US20030138772 SEQ ID NO: 8 |
| AAV3.3b | 34 | US20030138772 SEQ ID NO: 72 |
| AAV3-3 | 35 | US20150315612 SEQ ID NO: 200 |
| AAV3-3 | 36 | US20150315612 SEQ ID NO: 217 |
| AAV3a | 37 | U.S. Pat. No. 6,156,303 SEQ ID NO: 5 |
| AAV3a | 38 | U.S. Pat. No. 6,156,303 SEQ ID NO: 9 |
| AAV3b | 39 | U.S. Pat. No. 6,156,303 SEQ ID NO: 6 |
| AAV3b | 40 | U.S. Pat. No. 6,156,303 SEQ ID NO: 10 |
| AAV3b | 41 | U.S. Pat. No. 6,156,303 SEQ ID NO: 1 |
| AAV4 | 42 | US20140348794 SEQ ID NO: 17 |
| AAV4 | 43 | US20140348794 SEQ ID NO: 5 |
| AAV4 | 44 | US20140348794 SEQ ID NO: 3 |
| AAV4 | 45 | US20140348794 SEQ ID NO: 14 |
| AAV4 | 46 | US20140348794 SEQ ID NO: 15 |
| AAV4 | 47 | US20140348794 SEQ ID NO: 19 |
| AAV4 | 48 | US20140348794 SEQ ID NO: 12 |
| AAV4 | 49 | US20140348794 SEQ ID NO: 13 |
| AAV4 | 50 | US20140348794 SEQ ID NO: 7 |
| AAV4 | 51 | US20140348794 SEQ ID NO: 8 |
| AAV4 | 52 | US20140348794 SEQ ID NO: 9 |
| AAV4 | 53 | US20140348794 SEQ ID NO: 2 |
| AAV4 | 54 | US20140348794 SEQ ID NO: 10 |
| AAV4 | 55 | US20140348794 SEQ ID NO: 11 |
| AAV4 | 56 | US20140348794 SEQ ID NO: 18 |
| AAV4 | 57 | US20030138772 SEQ ID NO: 63, |
| | | US20160017295 SEQ ID NO: 4, |
| | | US20140348794 SEQ ID NO: 4 |
| AAV4 | 58 | US20140348794 SEQ ID NO: 16 |
| AAV4 | 59 | US20140348794 SEQ ID NO: 20 |
| AAV4 | 60 | US20140348794 SEQ ID NO: 6 |
| AAV4 | 61 | US20140348794 SEQ ID NO: 1 |
| AAV42.2 | 62 | US20030138772 SEQ ID NO: 9 |
| AAV42.2 | 63 | US20030138772 SEQ ID NO: 102 |
| AAV42.3b | 64 | US20030138772 SEQ ID NO: 36 |
| AAV42.3B | 65 | US20030138772 SEQ ID NO: 107 |
| AAV42.4 | 66 | US20030138772 SEQ ID NO: 33 |
| AAV42.4 | 67 | US20030138772 SEQ ID NO: 88 |
| AAV42.8 | 68 | US20030138772 SEQ ID NO: 27 |
| AAV42.8 | 69 | US20030138772 SEQ ID NO: 85 |
| AAV43.1 | 70 | US20030138772 SEQ ID NO: 39 |
| AAV43.1 | 71 | US20030138772 SEQ ID NO: 92 |
| AAV43.12 | 72 | US20030138772 SEQ ID NO: 41 |
| AAV43.12 | 73 | US20030138772 SEQ ID NO: 93 |
| AAV43.20 | 74 | US20030138772 SEQ ID NO: 42 |
| AAV43.20 | 75 | US20030138772 SEQ ID NO: 99 |
| AAV43.21 | 76 | US20030138772 SEQ ID NO: 43 |
| AAV43.21 | 77 | US20030138772 SEQ ID NO: 96 |
| AAV43.23 | 78 | US20030138772 SEQ ID NO: 44 |
| AAV43.23 | 79 | US20030138772 SEQ ID NO: 98 |
| AAV43.25 | 80 | US20030138772 SEQ ID NO: 45 |
| AAV43.25 | 81 | US20030138772 SEQ ID NO: 97 |
| AAV43.5 | 82 | US20030138772 SEQ ID NO: 40 |
| AAV43.5 | 83 | US20030138772 SEQ ID NO: 94 |
| AAV4-4 | 84 | US20150315612 SEQ ID NO: 201 |
| AAV4-4 | 85 | US20150315612 SEQ ID NO: 218 |
| AAV44.1 | 86 | US20030138772 SEQ ID NO: 46 |
| AAV44.1 | 87 | US20030138772 SEQ ID NO: 79 |
| AAV44.5 | 88 | US20030138772 SEQ ID NO: 47 |
| AAV44.5 | 89 | US20030138772 SEQ ID NO: 80 |
| AAV4407 | 90 | US20150315612 SEQ ID NO: 90 |
| AAV5 | 91 | U.S. Pat. No. 7,427,396 SEQ ID NO: 1 |
| AAV5 | 92 | US20030138772 SEQ ID NO: 114 |
| AAV5 | 93 | US20160017295 SEQ ID NO: 5, |
| | | U.S. Pat. No. 7,427,396 SEQ ID NO: 2, |
| | | US20150315612 SEQ ID NO: 216 |
| AAV5 | 94 | US20150315612 SEQ ID NO: 199 |
| AAV6 | 95 | US20150159173 SEQ ID NO: 13 |
| AAV6 | 96 | US20030138772 SEQ ID NO: 65, |
| | | US20150159173 SEQ ID NO: 29, |
| | | US20160017295 SEQ ID NO: 6, |
| | | U.S. Pat. No. 6,156,303 SEQ ID NO: 7 |
| AAV6 | 97 | U.S. Pat. No. 6,156,303 SEQ ID NO: 11 |
| AAV6 | 98 | U.S. Pat. No. 6,156,303 SEQ ID NO: 2 |
| AAV6 | 99 | US20150315612 SEQ ID NO: 203 |
| AAV6 | 100 | US20150315612 SEQ ID NO: 220 |
| AAV6.1 | 101 | US20150159173 |
| AAV6.12 | 102 | US20150159173 |
| AAV6.2 | 103 | US20150159173 |
| AAV7 | 104 | US20150159173 SEQ ID NO: 14 |
| AAV7 | 105 | US20150315612 SEQ ID NO: 183 |
| AAV7 | 106 | US20030138772 SEQ ID NO: 2, |
| | | US20150159173 SEQ ID NO: 30, |
| | | US20150315612 SEQ ID NO: 181, |
| | | US20160017295 SEQ ID NO: 7 |
| AAV7 | 107 | US20030138772 SEQ ID NO: 3 |
| AAV7 | 108 | US20030138772 SEQ ID NO: 1, |
| | | US20150315612 SEQ ID NO: 180 |
| AAV7 | 109 | US20150315612 SEQ ID NO: 213 |
| AAV7 | 110 | US20150315612 SEQ ID NO: 222 |
| AAV8 | 111 | US20150159173 SEQ ID NO: 15 |
| AAV8 | 112 | US20150376240 SEQ ID NO: 7 |
| AAV8 | 113 | US20030138772 SEQ ID NO: 4, |
| | | US20150315612 SEQ ID NO: 182 |
| AAV8 | 114 | US20030138772 SEQ ID NO: 95, |
| | | US20140359799 SEQ ID NO: 1, |
| | | US20150159173 SEQ ID NO: 31, |
| | | US20160017295 SEQ ID NO: 8, |
| | | U.S. Pat. No. 7,198,951 SEQ ID NO: 7, |
| | | US20150315612 SEQ ID NO: 223 |
| AAV8 | 115 | US20150376240 SEQ ID NO: 8 |
| AAV8 | 116 | US20150315612 SEQ ID NO: 214 |
| AAV-8b | 117 | US20150376240 SEQ ID NO: 5 |
| AAV-8b | 118 | US20150376240 SEQ ID NO: 3 |
| AAV-8h | 119 | US20150376240 SEQ ID NO: 6 |
| AAV-8h | 120 | US20150376240 SEQ ID NO: 4 |
| AAV9 | 121 | US20030138772 SEQ ID NO: 5 |
| AAV9 | 122 | U.S. Pat. No. 7,198,951 SEQ ID NO: 1 |
| AAV9 | 123 | US20160017295 SEQ ID NO: 9 |
| AAV9 | 124 | US20030138772 SEQ ID NO: 100, |
| | | U.S. Pat. No. 7,198,951 SEQ ID NO: 2 |
| AAV9 | 125 | U.S. Pat. No. 7,198,951 SEQ ID NO: 3 |
| AAV9 | 126 | U.S. Pat. No. 7,906,111 SEQ ID NO: 3; |
| (AAVhu.14) | | WO2015038958 SEQ ID NO: 11 |
| AAV9 | 127 | U.S. Pat. No. 7,906,111 SEQ ID NO: 123: |
| (AAVhu.14) | | WO2015038958 SEQ ID NO: 2 |
| AAVA3.1 | 128 | US20030138772 SEQ ID NO: 120 |
| AAVA3.3 | 129 | US20030138772 SEQ ID NO: 57 |
| AAVA3.3 | 130 | US20030138772 SEQ ID NO: 66 |
| AAVA3.4 | 131 | US20030138772 SEQ ID NO: 54 |
| AAVA3.4 | 132 | US20030138772 SEQ ID NO: 68 |
| AAVA3.5 | 133 | US20030138772 SEQ ID NO: 55 |
| AAVA3.5 | 134 | US20030138772 SEQ ID NO: 69 |
| AAVA3.7 | 135 | US20030138772 SEQ ID NO: 56 |
| AAVA3.7 | 136 | US20030138772 SEQ ID NO: 67 |
| AAV29.3 | 137 | US20030138772 SEQ ID NO: 11 |
| (AAVbb.1) | | |
| AAVC2 | 138 | US20030138772 SEQ ID NO: 61 |
| AAVCh.5 | 139 | US20150159173 SEQ ID NO: 46, |
| | | US20150315612 SEQ ID NO: 234 |
| AAVcy.2 | 140 | US20030138772 SEQ ID NO: 15 |
| (AAV13.3) | | |
| AAV24.1 | 141 | US20030138772 SEQ ID NO: 101 |
| AAVcy.3 | 142 | US20030138772 SEQ ID NO: 16 |
| (AAV24.1) | | |
| AAV27.3 | 143 | US20030138772 SEQ ID NO: 104 |
| AAVcy.4 | 144 | US20030138772 SEQ ID NO: 17 |
| (AAV27.3) | | |
| AAVcy.5 | 145 | US20150315612 SEQ ID NO: 227 |
| AAV7.2 | 146 | US20030138772 SEQ ID NO: 103 |
| AAVcy.5 | 147 | US20030138772 SEQ ID NO: 18 |
| (AAV7.2) | | |
| AAV16.3 | 148 | US20030138772 SEQ ID NO: 105 |
| AAVcy.6 | 149 | US20030138772 SEQ ID NO: 10 |
| (AAV16.3) | | |
| AAVcy.5 | 150 | US20150159173 SEQ ID NO: 8 |
| AAVcy.5 | 151 | US20150159173 SEQ ID NO: 24 |
| AAVCy.5R1 | 152 | US20150159173 |
| AAVCy.5R2 | 153 | US20150159173 |
| AAVCy.5R3 | 154 | US20150159173 |
| AAVCy.5R4 | 155 | US20150159173 |
| AAVDJ | 156 | US20140359799 SEQ ID NO: 3, |
| | | U.S. Pat. No. 7,588,772 SEQ ID NO: 2 |
| AAVDJ | 157 | US20140359799 SEQ ID NO: 2, |
| | | U.S. Pat. No. 7,588,772 SEQ ID NO: 1 |
| AAVDJ-8 | 158 | U.S. Pat. No. 7,588,772; Grimm et al 2008 |
| AAVDJ-8 | 159 | U.S. Pat. No. 7,588,772; Grimm et al 2008 |
| AAVF5 | 160 | US20030138772 SEQ ID NO: 110 |
| AAVH2 | 161 | US20030138772 SEQ ID NO: 26 |
| AAVH6 | 162 | US20030138772 SEQ ID NO: 25 |
| AAVhE1.1 | 163 | U.S. Pat. No. 9,233,131 SEQ ID NO: 44 |
| AAVhEr1.14 | 164 | U.S. Pat. No. 9,233,131 SEQ ID NO: 46 |
| AAVhEr1.16 | 165 | U.S. Pat. No. 9,233,131 SEQ ID NO: 48 |
| AAVhEr1.18 | 166 | U.S. Pat. No. 9,233,131 SEQ ID NO: 49 |
| AAVhEr1.23 | 167 | U.S. Pat. No. 9,233,131 SEQ ID NO: 53 |
| (AAVhEr2.29) | | |
| AAVhEr1.35 | 168 | U.S. Pat. No. 9,233,131 SEQ ID NO: 50 |
| AAVhEr1.36 | 169 | U.S. Pat. No. 9,233,131 SEQ ID NO: 52 |
| AAVhEr1.5 | 170 | U.S. Pat. No. 9,233,131 SEQ ID NO: 45 |
| AAVhEr1.7 | 171 | U.S. Pat. No. 9,233,131 SEQ ID NO: 51 |
| AAVhEr1.8 | 172 | U.S. Pat. No. 9,233,131 SEQ ID NO: 47 |
| AAVhEr2.16 | 173 | U.S. Pat. No. 9,233,131 SEQ ID NO: 55 |
| AAVhEr2.30 | 174 | U.S. Pat. No. 9,233,131 SEQ ID NO: 56 |
| AAVhEr2.31 | 175 | U.S. Pat. No. 9,233,131 SEQ ID NO: 58 |
| AAVhEr2.36 | 176 | U.S. Pat. No. 9,233,131 SEQ ID NO: 57 |
| AAVhEr2.4 | 177 | U.S. Pat. No. 9,233,131 SEQ ID NO: 54 |
| AAVhEr3.1 | 178 | U.S. Pat. No. 9,233,131 SEQ ID NO: 59 |
| AAVhu.1 | 179 | US20150315612 SEQ ID NO: 46 |
| AAVhu.1 | 180 | US20150315612 SEQ ID NO: 144 |
| AAVhu.10 | 181 | US20150315612 SEQ ID NO: 56 |
| (AAV16.8) | | |
| AAVhu.10 | 182 | US20150315612 SEQ ID NO: 156 |
| (AAV16.8) | | |
| AAVhu.11 | 183 | US20150315612 SEQ ID NO: 57 |
| (AAV16.12) | | |
| AAVhu.11 | 184 | US20150315612 SEQ ID NO: 153 |
| (AAV16.12) | | |
| AAVhu.12 | 185 | US20150315612 SEQ ID NO: 59 |
| AAVhu.12 | 186 | US20150315612 SEQ ID NO: 154 |
| AAVhu.13 | 187 | US20150159173 SEQ ID NO: 16, |
| | | US20150315612 SEQ ID NO: 71 |
| AAVhu.13 | 188 | US20150159173 SEQ ID NO: 32, |
| | | US20150315612 SEQ ID NO: 129 |
| AAVhu.136.1 | 189 | US20150315612 SEQ ID NO: 165 |
| AAVhu.140.1 | 190 | US20150315612 SEQ ID NO: 166 |
| AAVhu.140.2 | 191 | US20150315612 SEQ ID NO: 167 |
| AAVhu.145.6 | 192 | US20150315612 SEQ ID NO: 178 |
| AAVhu.15 | 193 | US20150315612 SEQ ID NO: 147 |
| AAVhu.15 | 194 | US20150315612 SEQ ID NO: 50 |
| (AAV33.4) | | |
| AAVhu.156.1 | 195 | US20150315612 SEQ ID NO: 179 |
| AAVhu.16 | 196 | US20150315612 SEQ ID NO: 148 |
| AAVhu.16 | 197 | US20150315612 SEQ ID NO: 51 |
| (AAV33.8) | | |
| AAVhu.17 | 198 | US20150315612 SEQ ID NO: 83 |
| AAVhu.17 | 199 | US20150315612 SEQ ID NO: 4 |
| (AAV33.12) | | |
| AAVhu.172.1 | 200 | US20150315612 SEQ ID NO: 171 |
| AAVhu.172.2 | 201 | US20150315612 SEQ ID NO: 172 |
| AAVhu.173.4 | 202 | US20150315612 SEQ ID NO: 173 |
| AAVhu.173.8 | 203 | US20150315612 SEQ ID NO: 175 |
| AAVhu.18 | 204 | US20150315612 SEQ ID NO: 52 |
| AAVhu.18 | 205 | US20150315612 SEQ ID NO: 149 |
| AAVhu.19 | 206 | US20150315612 SEQ ID NO: 62 |
| AAVhu.19 | 207 | US20150315612 SEQ ID NO: 133 |
| AAVhu.2 | 208 | US20150315612 SEQ ID NO: 48 |
| AAVhu.2 | 209 | US20150315612 SEQ ID NO: 143 |
| AAVhu.20 | 210 | US20150315612 SEQ ID NO: 63 |
| AAVhu.20 | 213 | US20150315612 SEQ ID NO: 134 |
| AAVhu.21 | 212 | US20150315612 SEQ ID NO: 65 |
| AAVhu.21 | 213 | US20150315612 SEQ ID NO: 135 |
| AAVhu.22 | 214 | US20150315612 SEQ ID NO: 67 |
| AAVhu.22 | 215 | US20150315612 SEQ ID NO: 138 |
| AAVhu.23 | 216 | US20150315612 SEQ ID NO: 60 |
| AAVhu.23.2 | 217 | US20150315612 SEQ ID NO: 137 |
| AAVhu.24 | 218 | US20150315612 SEQ ID NO: 66 |
| AAVhu.24 | 219 | US20150315612 SEQ ID NO: 136 |
| AAVhu.25 | 220 | US20150315612 SEQ ID NO: 49 |
| AAVhu.25 | 221 | US20150315612 SEQ ID NO: 146 |
| AAVhu.26 | 222 | US20150159173 SEQ ID NO: 17, |
| | | US20150315612 SEQ ID NO: 61 |
| AAVhu.26 | 223 | US20150159173 SEQ ID NO: 33, |
| | | US20150315612 SEQ ID NO: 139 |
| AAVhu.27 | 224 | US20150315612 SEQ ID NO: 64 |
| AAVhu.27 | 225 | US20150315612 SEQ ID NO: 140 |
| AAVhu.28 | 226 | US20150315612 SEQ ID NO: 68 |
| AAVhu.28 | 227 | US20150315612 SEQ ID NO: 130 |
| AAVhu.29 | 228 | US20150315612 SEQ ID NO: 69 |
| AAVhu.29 | 229 | US20150159173 SEQ ID NO: 42, |
| | | US20150315612 SEQ ID NO: 132 |
| AAVhu.29 | 230 | US20150315612 SEQ ID NO: 225 |
| AAVhu.29R | 231 | US20150159173 |
| AAVhu.3 | 232 | US20150315612 SEQ ID NO: 44 |
| AAVhu.3 | 233 | US20150315612 SEQ ID NO: 145 |
| AAVhu.30 | 234 | US20150315612 SEQ ID NO: 70 |
| AAVhu.30 | 235 | US20150315612 SEQ ID NO: 131 |
| AAVhu.31 | 236 | US20150315612 SEQ ID NO: 1 |
| AAVhu.31 | 237 | US20150315612 SEQ ID NO: 121 |
| AAVhu.32 | 238 | US20150315612 SEQ ID NO: 2 |
| AAVhu.32 | 239 | US20150315612 SEQ ID NO: 122 |
| AAVhu.33 | 240 | US20150315612 SEQ ID NO: 75 |
| AAVhu.33 | 241 | US20150315612 SEQ ID NO: 124 |
| AAVhu.34 | 242 | US20150315612 SEQ ID NO: 72 |
| AAVhu.34 | 243 | US20150315612 SEQ ID NO: 125 |
| AAVhu.35 | 244 | US20150315612 SEQ ID NO: 73 |
| AAVhu.35 | 245 | US20150315612 SEQ ID NO: 164 |
| AAVhu.36 | 246 | US20150315612 SEQ ID NO: 74 |
| AAVhu.36 | 247 | US20150315612 SEQ ID NO: 126 |
| AAVhu.37 | 248 | US20150159173 SEQ ID NO: 34, |
| | | US20150315612 SEQ ID NO: 88 |
| AAVhu.37 | 249 | US20150315612 SEQ ID NO: 10, |
| (AAV106.1) | | US20150159173 SEQ ID NO: 18 |
| AAVhu.38 | 250 | US20150315612 SEQ ID NO: 161 |
| AAVhu.39 | 251 | US20150315612 SEQ ID NO: 102 |
| AAVhu.39 | 252 | US20150315612 SEQ ID NO: 24 |
| (AAVLG-9) | | |
| AAVhu.4 | 253 | US20150315612 SEQ ID NO: 47 |
| AAVhu.4 | 254 | US20150315612 SEQ ID NO: 141 |
| AAVhu.40 | 255 | US20150315612 SEQ ID NO: 87 |
| AAVhu.40 | 256 | US20150315612 SEQ ID NO: 11 |
| (AAV114.3) | | |
| AAVhu.41 | 257 | US20150315612 SEQ ID NO: 91 |
| AAVhu.41 | 258 | US20150315612 SEQ ID NO: 6 |
| (AAV127.2) | | |
| AAVhu.42 | 259 | US20150315612 SEQ ID NO: 85 |
| AAVhu.42 | 260 | US20150315612 SEQ ID NO: 8 |
| (AAV127.5) | | |
| AAVhu.43 | 261 | US20150315612 SEQ ID NO: 160 |
| AAVhu.43 | 262 | US20150315612 SEQ ID NO: 236 |
| AAVhu.43 | 263 | US20150315612 SEQ ID NO: 80 |
| (AAV128.1) | | |
| AAVhu.44 | 264 | US20150159173 SEQ ID NO: 45, |
| | | US20150315612 SEQ ID NO: 158 |
| AAVhu.44 | 265 | US20150315612 SEQ ID NO: 81 |
| (AAV128.3) | | |
| AAVhu.44R1 | 266 | US20150159173 |
| AAVhu.44R2 | 267 | US20150159173 |
| AAVhu.44R3 | 268 | US20150159173 |
| AAVhu.45 | 269 | US20150315612 SEQ ID NO: 76 |
| AAVhu.45 | 270 | US20150315612 SEQ ID NO: 127 |
| AAVhu.46 | 271 | US20150315612 SEQ ID NO: 82 |
| AAVhu.46 | 272 | US20150315612 SEQ ID NO: 159 |
| AAVhu.46 | 273 | US20150315612 SEQ ID NO: 224 |
| AAVhu.47 | 274 | US20150315612 SEQ ID NO: 77 |
| AAVhu.47 | 275 | US20150315612 SEQ ID NO: 128 |
| AAVhu.48 | 276 | US20150159173 SEQ ID NO: 38 |
| AAVhu.48 | 277 | US20150315612 SEQ ID NO: 157 |
| AAVhu.48 | 278 | US20150315612 SEQ ID NO: 78 |
| (AAV130.4) | | |
| AAVhu.48R1 | 279 | US20150159173 |
| AAVhu.48R2 | 280 | US20150159173 |
| AAVhu.48R3 | 281 | US20150159173 |
| AAVhu.49 | 282 | US20150315612 SEQ ID NO: 209 |
| AAVhu.49 | 283 | US20150315612 SEQ ID NO: 189 |
| AAVhu.5 | 284 | US20150315612 SEQ ID NO: 45 |
| AAVhu.5 | 285 | US20150315612 SEQ ID NO: 142 |
| AAVhu.51 | 286 | US20150315612 SEQ ID NO: 208 |
| AAVhu.51 | 287 | US20150315612 SEQ ID NO: 190 |
| AAVhu.52 | 288 | US20150315612 SEQ ID NO: 210 |
| AAVhu.52 | 289 | US20150315612 SEQ ID NO: 191 |
| AAVhu.53 | 290 | US20150159173 SEQ ID NO: 19 |
| AAVhu.53 | 291 | US20150159173 SEQ ID NO: 35 |
| AAVhu.53 | 292 | US20150315612 SEQ ID NO: 176 |
| (AAV145.1) | | |
| AAVhu.54 | 293 | US20150315612 SEQ ID NO: 188 |
| AAVhu.54 | 294 | US20150315612 SEQ ID NO: 177 |
| (AAV145.5) | | |
| AAVhu.55 | 295 | US20150315612 SEQ ID NO: 187 |
| AAVhu.56 | 296 | US20150315612 SEQ ID NO: 205 |
| AAVhu.56 | 297 | US20150315612 SEQ ID NO: 168 |
| (AAV145.6) | | |
| AAVhu.56 | 298 | US20150315612 SEQ ID NO: 192 |
| (AAV145.6) | | |
| AAVhu.57 | 299 | US20150315612 SEQ ID NO: 206 |
| AAVhu.57 | 300 | US20150315612 SEQ ID NO: 169 |
| AAVhu.57 | 301 | US20150315612 SEQ ID NO: 193 |
| AAVhu.58 | 302 | US20150315612 SEQ ID NO: 207 |
| AAVhu.58 | 303 | US20150315612 SEQ ID NO: 194 |
| AAVhu.6 | 304 | US20150315612 SEQ ID NO: 5 |
| (AAV3.1) | | |
| AAVhu.6 | 305 | US20150315612 SEQ ID NO: 84 |
| (AAV3.1) | | |
| AAVhu.60 | 306 | US20150315612 SEQ ID NO: 184 |
| AAVhu.60 | 307 | US20150315612 SEQ ID NO: 170 |
| (AAV161.10) | | |
| AAVhu.61 | 308 | US20150315612 SEQ ID NO: 185 |
| AAVhu.61 | 309 | US20150315612 SEQ ID NO: 174 |
| (AAV161.6) | | |
| AAVhu.63 | 310 | US20150315612 SEQ ID NO: 204 |
| AAVhu.63 | 311 | US20150315612 SEQ ID NO: 195 |
| AAVhu.64 | 312 | US20150315612 SEQ ID NO: 212 |
| AAVhu.64 | 313 | US20150315612 SEQ ID NO: 196 |
| AAVhu.66 | 314 | US20150315612 SEQ ID NO: 197 |
| AAVhu.67 | 315 | US20150315612 SEQ ID NO: 215 |
| AAVhu.67 | 316 | US20150315612 SEQ ID NO: 198 |
| AAVhu.7 | 317 | US20150315612 SEQ ID NO: 226 |
| AAVhu.7 | 318 | US20150315612 SEQ ID NO: 150 |
| AAVhu.7 | 319 | US20150315612 SEQ ID NO: 55 |
| (AAV7.3) | | |
| AAVhu.71 | 320 | US20150315612 SEQ ID NO: 79 |
| AAVhu.8 | 321 | US20150315612 SEQ ID NO: 53 |
| AAVhu.8 | 322 | US20150315612 SEQ ID NO: 12 |
| AAVhu.8 | 323 | US20150315612 SEQ ID NO: 151 |
| AAVhu.9 | 324 | US20150315612 SEQ ID NO: 58 |
| (AAV3.1) | | |
| AAVhu.9 | 325 | US20150315612 SEQ ID NO: 155 |
| (AAV3.1) | | |
| AAV-LK01 | 326 | US20150376607 SEQ ID NO: 2 |
| AAV-LK01 | 327 | US20150376607 SEQ ID NO: 29 |
| AAV-LK02 | 328 | US20150376607 SEQ ID NO: 3 |
| AAV-LK02 | 329 | US20150376607 SEQ ID NO: 30 |
| AAV-LK03 | 330 | US20150376607 SEQ ID NO: 4 |
| AAV-LK03 | 331 | WO2015121501 SEQ ID NO: 12, |
| | | US20150376607 SEQ ID NO: 31 |
| AAV-LK04 | 332 | US20150376607 SEQ ID NO: 5 |
| AAV-LK04 | 333 | US20150376607 SEQ ID NO: 32 |
| AAV-LK05 | 334 | US20150376607 SEQ ID NO: 6 |
| AAV-LK05 | 335 | US20150376607 SEQ ID NO: 33 |
| AAV-LK06 | 336 | US20150376607 SEQ ID NO: 7 |
| AAV-LK06 | 337 | US20150376607 SEQ ID NO: 34 |
| AAV-LK07 | 338 | US20150376607 SEQ ID NO: 8 |
| AAV-LK08 | 339 | US20150376607 SEQ ID NO: 35 |
| AAV-LK08 | 340 | US20150376607 SEQ ID NO: 9 |
| AAV-LK08 | 341 | US20150376607 SEQ ID NO: 36 |
| AAV-LK09 | 342 | US20150376607 SEQ ID NO: 10 |
| AAV-LK09 | 343 | US20150376607 SEQ ID NO: 37 |
| AAV-LK10 | 344 | US20150376607 SEQ ID NO: 11 |
| AAV-LK10 | 345 | US20150376607 SEQ ID NO: 38 |
| AAV-LK11 | 346 | US20150376607 SEQ ID NO: 12 |
| AAV-LK11 | 347 | US20150376607 SEQ ID NO: 39 |
| AAV-LK12 | 348 | US20150376607 SEQ ID NO: 13 |
| AAV-LK12 | 349 | US20150376607 SEQ ID NO: 40 |
| AAV-LK13 | 350 | US20150376607 SEQ ID NO: 14 |
| AAV-LK13 | 351 | US20150376607 SEQ ID NO: 41 |
| AAV-LK14 | 352 | US20150376607 SEQ ID NO: 15 |
| AAV-LK14 | 353 | US20150376607 SEQ ID NO: 42 |
| AAV-LK15 | 354 | US20150376607 SEQ ID NO: 16 |
| AAV-LK15 | 355 | US20150376607 SEQ ID NO: 43 |
| AAV-LK16 | 356 | US20150376607 SEQ ID NO: 17 |
| AAV-LK16 | 357 | US20150376607 SEQ ID NO: 44 |
| AAV-LK17 | 358 | US20150376607 SEQ ID NO: 18 |
| AAV-LK17 | 359 | US20150376607 SEQ ID NO: 45 |
| AAV-LK18 | 360 | US20150376607 SEQ ID NO: 19 |
| AAV-LK18 | 361 | US20150376607 SEQ ID NO: 46 |
| AAV-LK19 | 362 | US20150376607 SEQ ID NO: 20 |
| AAV-LK19 | 363 | US20150376607 SEQ ID NO: 47 |
| AAV-PAEC | 364 | US20150376607 SEQ ID NO: 1 |
| AAV-PAEC | 365 | US20150376607 SEQ ID NO: 48 |
| AAV- | 366 | US20150376607 SEQ ID NO: 26 |
| PAEC11 | | |
| AAV- | 367 | US20150376607 SEQ ID NO: 54 |
| PAEC11 | | |
| AAV- | 368 | US20150376607 SEQ ID NO: 27 |
| PAEC12 | | |
| AAV- | 369 | US20150376607 SEQ ID NO: 51 |
| PAEC12 | | |
| AAV- | 370 | US20150376607 SEQ ID NO: 28 |
| PAEC13 | | |
| AAV- | 371 | US20150376607 SEQ ID NO: 49 |
| PAEC13 | | |
| AAV-PAEC2 | 372 | US20150376607 SEQ ID NO: 21 |
| AAV-PAEC2 | 373 | US20150376607 SEQ ID NO: 56 |
| AAV-PAEC4 | 374 | US20150376607 SEQ ID NO: 22 |
| AAV-PAEC4 | 375 | US20150376607 SEQ ID NO: 55 |
| AAV-PAEC6 | 376 | US20150376607 SEQ ID NO: 23 |
| AAV-PAEC6 | 377 | US20150376607 SEQ ID NO: 52 |
| AAV-PAEC7 | 378 | US20150376607 SEQ ID NO: 24 |
| AAV-PAEC7 | 379 | US20150376607 SEQ ID NO: 53 |
| AAV-PAEC8 | 380 | US20150376607 SEQ ID NO: 25 |
| AAV-PAEC8 | 381 | US20150376607 SEQ ID NO: 50 |
| AAVpi.1 | 382 | US20150315612 SEQ ID NO: 28 |
| AAVpi.1 | 383 | US20150315612 SEQ ID NO: 93 |
| AAVpi.2 | 384 | US20150315612 SEQ ID NO: 30 |
| AAVpi.2 | 385 | US20150315612 SEQ ID NO: 95 |
| AAVpi.3 | 386 | US20150315612 SEQ ID NO: 29 |
| AAVpi.3 | 387 | US20150315612 SEQ ID NO: 94 |
| AAVrh.10 | 388 | US20150159173 SEQ ID NO: 9 |
| AAVrh.10 | 389 | US20150159173 SEQ ID NO: 25 |
| AAV44.2 | 390 | US20030138772 SEQ ID NO: 59 |
| AAVrh.10 | 391 | US20030138772 SEQ ID NO: 81 |
| (AAV44.2) | | |
| AAV42.1B | 392 | US20030138772 SEQ ID NO: 90 |
| AAVrh.12 | 393 | US20030138772 SEQ ID NO: 30 |
| (AAV42.1b) | | |
| AAVrh.13 | 394 | US20150159173 SEQ ID NO: 10 |
| AAVrh.13 | 395 | US20150159173 SEQ ID NO: 26 |
| AAVrh.13 | 396 | US20150315612 SEQ ID NO: 228 |
| AAVrh.13R | 397 | US20150159173 |
| AAV42.3A | 398 | US20030138772 SEQ ID NO: 87 |
| AAVrh.14 | 399 | US20030138772 SEQ ID NO: 32 |
| (AAV42.3a) | | |
| AAV42.5A | 400 | US20030138772 SEQ ID NO: 89 |
| AAVrh.17 | 401 | US20030138772 SEQ ID NO: 34 |
| (AAV42.5a) | | |
| AAV42.5B | 402 | US20030138772 SEQ ID NO: 91 |
| AAVrh.18 | 403 | US20030138772 SEQ ID NO: 29 |
| (AAV42.5b) | | |
| AAV42.6B | 404 | US20030138772 SEQ ID NO: 112 |
| AAVrh.19 | 405 | US20030138772 SEQ ID NO: 38 |
| (AAV42.6b) | | |
| AAVrh.2 | 406 | US20150159173 SEQ ID NO: 39 |
| AAVrh.2 | 407 | US20150315612 SEQ ID NO: 231 |
| AAVrh.20 | 408 | US20150159173 SEQ ID NO: 1 |
| AAV42.10 | 409 | US20030138772 SEQ ID NO: 106 |
| AAVrh.21 | 410 | US20030138772 SEQ ID NO: 35 |
| (AAV42.10) | | |
| AAV42.11 | 411 | US20030138772 SEQ ID NO: 108 |
| AAVrh.22 | 412 | US20030138772 SEQ ID NO: 37 |
| (AAV42.11) | | |
| AAV42.12 | 413 | US20030138772 SEQ ID NO: 113 |
| AAVrh.23 | 414 | US20030138772 SEQ ID NO: 58 |
| (AAV42.12) | | |
| AAV42.13 | 415 | US20030138772 SEQ ID NO: 86 |
| AAVrh.24 | 416 | US20030138772 SEQ ID NO: 31 |
| (AAV42.13) | | |
| AAV42.15 | 417 | US20030138772 SEQ ID NO: 84 |
| AAVrh.25 | 418 | US20030138772 SEQ ID NO: 28 |
| (AAV42.15) | | |
| AAVrh.2R | 419 | US20150159173 |
| AAVrh.31 | 420 | US20030138772 SEQ ID NO: 48 |
| (AAV223.1) | | |
| AAVC1 | 421 | US20030138772 SEQ ID NO: 60 |
| AAVrh.32 | 422 | US20030138772 SEQ ID NO: 19 |
| (AAVC1) | | |
| AAVrh.32/33 | 423 | US20150159173 SEQ ID NO: 2 |
| AAVrh.33 | 424 | US20030138772 SEQ ID NO: 20 |
| (AAVC3) | | |
| AAVC5 | 425 | US20030138772 SEQ ID NO: 62 |
| AAVrh.34 | 426 | US20030138772 SEQ ID NO: 21 |
| (AAVC5) | | |
| AAVF1 | 427 | US20030138772 SEQ ID NO: 109 |
| AAVrh.35 | 428 | US20030138772 SEQ ID NO: 22 |
| (AAVF1) | | |
| AAVF3 | 429 | US20030138772 SEQ ID NO: 111 |
| AAVrh.36 | 430 | US20030138772 SEQ ID NO: 23 |
| (AAVF3) | | |
| AAVrh.37 | 431 | US20030138772 SEQ ID NO: 24 |
| AAVrh.37 | 432 | US20150159173 SEQ ID NO: 40 |
| AAVrh.37 | 433 | US20150315612 SEQ ID NO: 229 |
| AAVrh.37R2 | 434 | US20150159173 |
| AAVrh.38 | 435 | US20150315612 SEQ ID NO: 7 |
| (AAVLG-4) | | |
| AAVrh.38 | 436 | US20150315612 SEQ ID NO: 86 |
| (AAVLG-4) | | |
| AAVrh.39 | 437 | US20150159173 SEQ ID NO: 20, |
| | | US20150315612 SEQ ID NO: 13 |
| AAVrh.39 | 438 | US20150159173 SEQ ID NO: 3, |
| | | US20150159173 SEQ ID NO: 36, |
| | | US20150315612 SEQ ID NO: 89 |
| AAVrh.40 | 439 | US20150315612 SEQ ID NO: 92 |
| AAVrh.40 | 440 | US20150315612 SEQ ID NO: 14 |
| (AAVLG-10) | | |
| AAVrh.43 | 441 | US20150315612 SEQ ID NO: 43, |
| (AAVN721-8) | | US20150159173 SEQ ID NO: 21 |
| AAVrh.43 | 442 | US20150315612 SEQ ID NO: 163, |
| (AAVN721-8) | | US20150159173 SEQ ID NO: 37 |
| AAVrh.44 | 443 | US20150315612 SEQ ID NO: 34 |
| AAVrh.44 | 444 | US20150315612 SEQ ID NO: 111 |
| AAVrh.45 | 445 | US20150315612 SEQ ID NO: 41 |
| AAVrh.45 | 446 | US20150315612 SEQ ID NO: 109 |
| AAVrh.46 | 447 | US20150159173 SEQ ID NO: 22, |
| | | US20150315612 SEQ ID NO: 19 |
| AAVrh.46 | 448 | US20150159173 SEQ ID NO: 4, |
| | | US20150315612 SEQ ID NO: 101 |
| AAVrh.47 | 449 | US20150315612 SEQ ID NO: 38 |
| AAVrh.47 | 450 | US20150315612 SEQ ID NO: 118 |
| AAVrh.48 | 451 | US20150159173 SEQ ID NO: 44, |
| | | US20150315612 SEQ ID NO: 115 |
| AAVrh.48.1 | 452 | US20150159173 |
| AAVrh.48.1.2 | 453 | US20150159173 |
| AAVrh.48.2 | 454 | US20150159173 |
| AAVrh.48 | 455 | US20150315612 SEQ ID NO: 32 |
| (AAV1-7) | | |
| AAVrh.49 | 456 | US20150315612 SEQ ID NO: 25 |
| (AAV1-8) | | |
| AAVrh.49 | 457 | US20150315612 SEQ ID NO: 103 |
| (AAV1-8) | | |
| AAVrh.50 | 458 | US20150315612 SEQ ID NO: 23 |
| (AAV2-4) | | |
| AAVrh.50 | 459 | US20150315612 SEQ ID NO: 108 |
| (AAV2-4) | | |
| AAVrh.51 | 460 | US20150315612 SEQ ID NO: 22 |
| (AAV2-5) | | |
| AAVrh.51 | 461 | US20150315612 SEQ ID NO: 104 |
| (AAV2-5) | | |
| AAVrh.52 | 462 | US20150315612 SEQ ID NO: 18 |
| (AAV3-9) | | |
| AAVrh.52 | 463 | US20150315612 SEQ ID NO: 96 |
| (AAV3-9) | | |
| AAVrh.53 | 464 | US20150315612 SEQ ID NO: 97 |
| AAVrh.53 | 465 | US20150315612 SEQ ID NO: 17 |
| (AAV3-11) | | |
| AAVrh.53 | 466 | US20150315612 SEQ ID NO: 186 |
| (AAV3-11) | | |
| AAVrh.54 | 467 | US20150315612 SEQ ID NO: 40 |
| AAVrh.54 | 468 | US20150159173 SEQ ID NO: 49, |
| | | US20150315612 SEQ ID NO: 116 |
| AAVrh.55 | 469 | US20150315612 SEQ ID NO: 37 |
| AAVrh.55 | 470 | US20150315612 SEQ ID NO: 117 |
| (AAV4-19) | | |
| AAVrh.56 | 471 | US20150315612 SEQ ID NO: 54 |
| AAVrh.56 | 472 | US20150315612 SEQ ID NO: 152 |
| AAVrh.57 | 473 | US20150315612 SEQ ID NO: 26 |
| AAVrh.57 | 474 | US20150315612 SEQ ID NO: 105 |
| AAVrh.58 | 475 | US20150315612 SEQ ID NO: 27 |
| AAVrh.58 | 476 | US20150159173 SEQ ID NO: 48, |
| | | US20150315612 SEQ ID NO: 106 |
| AAVrh.58 | 477 | US20150315612 SEQ ID NO: 232 |
| AAVrh.59 | 478 | US20150315612 SEQ ID NO: 42 |
| AAVrh.59 | 479 | US20150315612 SEQ ID NO: 110 |
| AAVrh.60 | 480 | US20150315612 SEQ ID NO: 31 |
| AAVrh.60 | 481 | US20150315612 SEQ ID NO: 120 |
| AAVrh.61 | 482 | US20150315612 SEQ ID NO: 107 |
| AAVrh.61 | 483 | US20150315612 SEQ ID NO: 21 |
| (AAV2-3) | | |
| AAVrh.62 | 484 | US20150315612 SEQ ID NO: 33 |
| (AAV2-15) | | |
| AAVrh.62 | 485 | US20150315612 SEQ ID NO: 114 |
| (AAV2-15) | | |
| AAVrh.64 | 486 | US20150315612 SEQ ID NO: 15 |
| AAVrh.64 | 487 | US20150159173 SEQ ID NO: 43, |
| | | US20150315612 SEQ ID NO: 99 |
| AAVrh.64 | 488 | US20150315612 SEQ ID NO: 233 |
| AAVRh.64R1 | 489 | US20150159173 |
| AAVRh.64R2 | 490 | US20150159173 |
| AAVrh.65 | 491 | US20150315612 SEQ ID NO: 35 |
| AAVrh.65 | 492 | US20150315612 SEQ ID NO: 112 |
| AAVrh.67 | 493 | US20150315612 SEQ ID NO: 36 |
| AAVrh.67 | 494 | US20150315612 SEQ ID NO: 230 |
| AAVrh.67 | 495 | US20150159173 SEQ ID NO: 47, |
| | | US20150315612 SEQ ID NO: 113 |
| AAVrh.68 | 496 | US20150315612 SEQ ID NO: 16 |
| AAVrh.68 | 497 | US20150315612 SEQ ID NO: 100 |
| AAVrh.69 | 498 | US20150315612 SEQ ID NO: 39 |
| AAVrh.69 | 499 | US20150315612 SEQ ID NO: 119 |
| AAVrh.70 | 500 | US20150315612 SEQ ID NO: 20 |
| AAVrh.70 | 501 | US20150315612 SEQ ID NO: 98 |
| AAVrh.71 | 502 | US20150315612 SEQ ID NO: 162 |
| AAVrh.72 | 503 | US20150315612 SEQ ID NO: 9 |
| AAVrh.73 | 504 | US20150159173 SEQ ID NO: 5 |
| AAVrh.74 | 505 | US20150159173 SEQ ID NO: 6 |
| AAVrh.8 | 506 | US20150159173 SEQ ID NO: 41 |
| AAVrh.8 | 507 | US20150315612 SEQ ID NO: 235 |
| AAVrh.8R | 508 | US20150159173, |
| | | WO2015168666 SEQ ID NO: 9 |
| AAVrh.8R | 509 | WO2015168666 SEQ ID NO: 10 |
| A586R | | |
| mutant | | |
| AAVrh.8R | 510 | WO2015168666 SEQ ID NO: 11 |
| R533A | | |
| mutant | | |
| BAAV | 511 | U.S. Pat. No. 9,193,769 SEQ ID NO: 8 |
| (bovine | | |
| AAV) | | |
| BAAV | 512 | U.S. Pat. No. 9,193,769 SEQ ID NO: 10 |
| (bovine | | |
| AAV) | | |
| BAAV | 513 | U.S. Pat. No. 9,193,769 SEQ ID NO: 4 |
| (bovine | | |
| AAV) | | |
| BAAV | 514 | U.S. Pat. No. 9,193,769 SEQ ID NO: 2 |
| (bovine | | |
| AAV) | | |
| BAAV | 515 | U.S. Pat. No. 9,193,769 SEQ ID NO: 6 |
| (bovine | | |
| AAV) | | |
| BAAV | 516 | U.S. Pat. No. 9,193,769 SEQ ID NO: 1 |
| (bovine | | |
| AAV) | | |
| BAAV | 517 | U.S. Pat. No. 9,193,769 SEQ ID NO: 5 |
| (bovine | | |
| AAV) | | |
| BAAV | 518 | U.S. Pat. No. 9,193,769 SEQ ID NO: 3 |
| (bovine | | |
| AAV) | | |
| BAAV | 519 | U.S. Pat. No. 9,193,769 SEQ ID NO: 11 |
| (bovine | | |
| AAV) | | |
| BAAV | 520 | U.S. Pat. No. 7,427,396 SEQ ID NO: 5 |
| (bovine | | |
| AAV) | | |
| BAAV | 521 | U.S. Pat. No. 7,427,396 SEQ ID NO: 6 |
| (bovine | | |
| AAV) | | |
| BAAV | 522 | U.S. Pat. No. 9,193,769 SEQ ID NO: 7 |
| (bovine | | |
| AAV) | | |
| BAAV | 523 | U.S. Pat. No. 9,193,769 SEQ ID NO: 9 |
| (bovine | | |
| AAV) | | |
| BNP61 AAV | 524 | US20150238550 SEQ ID NO: 1 |
| BNP61 AAV | 525 | US20150238550 SEQ ID NO: 2 |
| BNP62 AAV | 526 | US20150238550 SEQ ID NO: 3 |
| BNP63 AAV | 527 | US20150238550 SEQ ID NO: 4 |
| caprine AAV | 528 | U.S. Pat. No. 7,427,396 SEQ ID NO: 3 |
| caprine AAV | 529 | U.S. Pat. No. 7,427,396 SEQ ID NO: 4 |
| true type | 530 | WO2015121501 SEQ ID NO: 2 |
| AAV | | |
| (ttAAV) | | |
| AAAV | 531 | U.S. Pat. No. 9,238,800 SEQ ID NO: 12 |
| (Avian AAV) | | |
| AAAV | 532 | U.S. Pat. No. 9,238,800 SEQ ID NO: 2 |
| (Avian AAV) | | |
| AAAV | 533 | U.S. Pat. No. 9,238,800 SEQ ID NO: 6 |
| (Avian AAV) | | |
| AAAV | 534 | U.S. Pat. No. 9,238,800 SEQ ID NO: 4 |
| (Avian AAV) | | |
| AAAV | 535 | U.S. Pat. No. 9,238,800 SEQ ID NO: 8 |
| (Avian AAV) | | |
| AAAV | 536 | U.S. Pat. No. 9,238,800 SEQ ID NO: 14 |
| (Avian AAV) | | |
| AAAV | 537 | U.S. Pat. No. 9,238,800 SEQ ID NO: 10 |
| (Avian AAV) | | |
| AAAV | 538 | U.S. Pat. No. 9,238,800 SEQ ID NO: 15 |
| (Avian AAV) | | |
| AAAV | 539 | U.S. Pat. No. 9,238,800 SEQ ID NO: 5 |
| (Avian AAV) | | |
| AAAV | 540 | U.S. Pat. No. 9,238,800 SEQ ID NO: 9 |
| (Avian AAV) | | |
| AAAV | 541 | U.S. Pat. No. 9,238,800 SEQ ID NO: 3 |
| (Avian AAV) | | |
| AAAV | 542 | U.S. Pat. No. 9,238,800 SEQ ID NO: 7 |
| (Avian AAV) | | |
| AAAV | 543 | U.S. Pat. No. 9,238,800 SEQ ID NO: 11 |
| (Avian AAV) | | |
| AAAV | 544 | U.S. Pat. No. 9,238,800 SEQ ID NO: 13 |
| (Avian AAV) | | |
| AAAV | 545 | U.S. Pat. No. 9,238,800 SEQ ID NO: 1 |
| (Avian AAV) | | |
| AAV Shuffle | 546 | US20160017295 SEQ ID NO: 23 |
| 100-1 | | |
| AAV Shuffle | 547 | US20160017295 SEQ ID NO: 11 |
| 100-1 | | |
| AAV Shuffle | 548 | US20160017295 SEQ ID NO: 37 |
| 100-2 | | |
| AAV Shuffle | 549 | US20160017295 SEQ ID NO: 29 |
| 100-2 | | |
| AAV Shuffle | 550 | US20160017295 SEQ ID NO: 24 |
| 100-3 | | |
| AAV Shuffle | 551 | US20160017295 SEQ ID NO: 12 |
| 100-3 | | |
| AAV Shuffle | 552 | US20160017295 SEQ ID NO: 25 |
| 100-7 | | |
| AAV Shuffle | 553 | US20160017295 SEQ ID NO: 13 |
| 100-7 | | |
| AAV Shuffle | 554 | US20160017295 SEQ ID NO: 34 |
| 10-2 | | |
| AAV Shuffle | 555 | US20160017295 SEQ ID NO: 26 |
| 10-2 | | |
| AAV Shuffle | 556 | US20160017295 SEQ ID NO: 35 |
| 10-6 | | |
| AAV Shuffle | 557 | US20160017295 SEQ ID NO: 27 |
| 10-6 | | |
| AAV Shuffle | 558 | US20160017295 SEQ ID NO: 36 |
| 10-8 | | |
| AAV Shuffle | 559 | US20160017295 SEQ ID NO: 28 |
| 10-8 | | |
| AAV SM | 560 | US20160017295 SEQ ID NO: 41 |
| 100-10 | | |
| AAV SM | 561 | US20160017295 SEQ ID NO: 33 |
| 100-10 | | |
| AAV SM | 562 | US20160017295 SEQ ID NO: 40 |
| 100-3 | | |
| AAV SM | 563 | US20160017295 SEQ ID NO: 32 |
| 100-3 | | |
| AAV SM | 564 | US20160017295 SEQ ID NO: 38 |
| 10-1 | | |
| AAV SM | 565 | US20160017295 SEQ ID NO: 30 |
| 10-1 | | |
| AAV SM | 566 | US20160017295 SEQ ID NO: 10 |
| 10-2 | | |
| AAV SM | 567 | US20160017295 SEQ ID NO: 22 |
| 10-2 | | |
| AAV SM | 568 | US20160017295 SEQ ID NO: 39 |
| 10-8 | | |
| AAV SM | 569 | US20160017295 SEQ ID NO: 31 |
| 10-8 | | |
| AAV SM | 560 | US20160017295 SEQ ID NO: 41 |
| 100-10 | | |
| AAV SM | 561 | US20160017295 SEQ ID NO: 33 |
| 100-10 | | |
| AAV SM | 562 | US20160017295 SEQ ID NO: 40 |
| 300-3 | | |
| AAV SM | 563 | US20160017295 SEQ ID NO: 32 |
| 100-3 | | |
| AAV SM | 564 | US20160017295 SEQ ID NO: 38 |
| 10-1 | | |
| AAV SM | 565 | US20160017295 SEQ ID NO: 30 |
| 10-1 | | |
| AAV SM | 566 | US20160017295 SEQ ID NO: 10 |
| 10-2 | | |
| AAV SM | 567 | US20160017295 SEQ ID NO: 22 |
| 10-2 | | |
| AAV SM | 568 | US20160017295 SEQ ID NO: 39 |
| 10-8 | | |
| AAV SM | 569 | US20160017295 SEQ ID NO: 31 |
| 10-8 | | |
| AAVF1/ | 570 | WO2016049230 SEQ ID NO: 20 |
| HSC1 | | |
| AAVF2/ | 571 | WO2016049230 SEQ ID NO: 21 |
| HSC2 | | |
| AAVF3/ | 572 | WO2016049230 SEQ ID NO: 22 |
| HSC3 | | |
| AAVF4/ | 573 | WO2016049230 SEQ ID NO: 23 |
| HSC4 | | |
| AAVF5/ | 574 | WO2016049230 SEQ ID NO: 25 |
| HSC5 | | |
| AAVF6/ | 575 | WO2016049230 SEQ ID NO: 24 |
| HSC6 | | |
| AAVF7/ | 576 | WO2016049230 SEQ ID NO: 27 |
| HSC | | |
| AAVF8/ | 577 | WO2016049230 SEQ ID NO: 28 |
| HSC8 | | |
| AAVF9/ | 578 | WO2016049230 SEQ ID NO: 29 |
| HSC9 | | |
| AAVF11/ | 579 | WO2016049230 SEQ ID NO: 26 |
| HSC11 | | |
| AAVF12/ | 580 | WO2016049230 SEQ ID NO: 30 |
| HSC12 | | |
| AAVF13/ | 581 | WO2016049230 SEQ ID NO: 31 |
| HSC13 | | |
| AAVF14/ | 582 | WO2016049230 SEQ ID NO: 32 |
| HSC14 | | |
| AAVF15/ | 583 | WO2016049230 SEQ ID NO: 33 |
| HSC15 | | |
| AAVF16/ | 584 | WO2016049230 SEQ ID NO: 34 |
| HSC16 | | |
| AAVF17/ | 585 | WO2016049230 SEQ ID NO: 35 |
| HSC17 | | |
| AAVF1/HSC1 | 586 | WO2016049230 SEQ ID NO: 2 |
| AAVF2/HSC2 | 587 | WO2016049230 SEQ ID NO: 3 |
| AAVF3/HSC3 | 588 | WO2016049230 SEQ ID NO: 5 |
| AAVF4/HSC4 | 589 | WO2016049230 SEQ ID NO: 6 |
| AAVF5/HSC5 | 590 | WO2016049230 SEQ ID NO: 11 |
| AAVF6/HSC6 | 591 | WO2016049230 SEQ ID NO: 7 |
| AAVF7/HSC7 | 592 | WO2016049230 SEQ ID NO: 8 |
| AAVF8/HSC8 | 593 | WO2016049230 SEQ ID NO: 9 |
| AAVF9/HSC9 | 594 | WO2016049230 SEQ ID NO: 10 |
| AAVF11/ | 595 | WO2016049230 SEQ ID NO: 4 |
| HSC11 | | |
| AAVF12/ | 596 | WO2016049230 SEQ ID NO: 12 |
| HSC12 | | |
| AAVF13/ | 597 | WO2016049230 SEQ ID NO: 14 |
| HSC13 | | |
| AAVF14/ | 598 | WO2016049230 SEQ ID NO: 15 |
| HSC14 | | |
| AAVF15/ | 599 | WO2016049230 SEQ ID NO: 16 |
| HSC15 | | |
| AAVF16/ | 600 | WO2016049230 SEQ ID NO: 17 |
| HSC16 | | |
| AAVF17/ | 601 | WO2016049230 SEQ ID NO: 13 |
| HSC17 | | |
| AAV CBr-E1 | 602 | U.S. Pat. No. 8,734,809 SEQ ID NO: 13 |
| AAV CBr-E2 | 603 | U.S. Pat. No. 8,734,809 SEQ ID NO: 14 |
| AAV CBr-E3 | 604 | U.S. Pat. No. 8,734,809 SEQ ID NO: 15 |
| AAV CBr-E4 | 605 | U.S. Pat. No. 8,734,809 SEQ ID NO: 16 |
| AAV CBr-E5 | 606 | U.S. Pat. No. 8,734,809 SEQ ID NO: 17 |
| AAV CBr-e5 | 607 | U.S. Pat. No. 8,734,809 SEQ ID NO: 18 |
| AAV CBr-E6 | 608 | U.S. Pat. No. 8,734,809 SEQ ID NO: 19 |
| AAV CBr-E7 | 609 | U.S. Pat. No. 8,734,809 SEQ ID NO: 20 |
| AAV CBr-E8 | 610 | U.S. Pat. No. 8,734,809 SEQ ID NO: 21 |
| AAV CLv-D1 | 611 | U.S. Pat. No. 8,734,809 SEQ ID NO: 22 |
| AAV CLv-D2 | 612 | U.S. Pat. No. 8,734,809 SEQ ID NO: 23 |
| AAV CLv-D3 | 613 | U.S. Pat. No. 8,734,809 SEQ ID NO: 24 |
| AAV CLv-D4 | 614 | U.S. Pat. No. 8,734,809 SEQ ID NO: 25 |
| AAV CLv-D5 | 615 | U.S. Pat. No. 8,734,809 SEQ ID NO: 26 |
| AAV CLv-D6 | 616 | U.S. Pat. No. 8,734,809 SEQ ID NO: 27 |
| AAV CLv-D7 | 617 | U.S. Pat. No. 8,734,809 SEQ ID NO: 28 |
| AAV CLv-D8 | 618 | U.S. Pat. No. 8,734,809 SEQ ID NO: 29 |
| AAV CLv-E1 | 619 | U.S. Pat. No. 8,734,809 SEQ ID NO: 13 |
| AAV CLv-R1 | 620 | U.S. Pat. No. 8,734,809 SEQ ID NO: 30 |
| AAV CLv-R2 | 621 | U.S. Pat. No. 8,734,809 SEQ ID NO: 31 |
| AAV CLv-R3 | 622 | U.S. Pat. No. 8,734,809 SEQ ID NO: 32 |
| AAV CLv-R4 | 623 | U.S. Pat. No. 8,734,809 SEQ ID NO: 33 |
| AAV CLv-R5 | 624 | U.S. Pat. No. 8,734,809 SEQ ID NO: 34 |
| AAV CLv-R6 | 625 | U.S. Pat. No. 8,734,809 SEQ ID NO: 35 |
| AAV CLv-R7 | 626 | U.S. Pat. No. 8,734,809 SEQ ID NO: 36 |
| AAV CLv-R8 | 627 | U.S. Pat. No. 8,734,809 SEQ ID NO: 37 |
| AAV CLv-R9 | 628 | U.S. Pat. No. 8,734,809 SEQ ID NO: 38 |
| AAV CLg-F1 | 629 | U.S. Pat. No. 8,734,809 SEQ ID NO: 39 |
| AAV CLg-F2 | 630 | U.S. Pat. No. 8,734,809 SEQ ID NO: 40 |
| AAV CLg-F3 | 631 | U.S. Pat. No. 8,734,809 SEQ ID NO: 41 |
| AAV CLg-F4 | 632 | U.S. Pat. No. 8,734,809 SEQ ID NO: 42 |
| AAV CLg-F5 | 633 | U.S. Pat. No. 8,734,809 SEQ ID NO: 43 |
| AAV CLg-F6 | 634 | U.S. Pat. No. 8,734,809 SEQ ID NO: 43 |
| AAV CLg-F7 | 635 | U.S. Pat. No. 8,734,809 SEQ ID NO: 44 |
| AAV CLg-F8 | 636 | U.S. Pat. No. 8,734,809 SEQ ID NO: 43 |
| AAV CSp-1 | 637 | U.S. Pat. No. 8,734,809 SEQ ID NO: 45 |
| AAV CSp-10 | 638 | U.S. Pat. No. 8,734,809 SEQ ID NO: 46 |
| AAV CSp-11 | 639 | U.S. Pat. No. 8,734,809 SEQ ID NO: 47 |
| AAV CSp-2 | 640 | U.S. Pat. No. 8,734,809 SEQ ID NO: 48 |
| AAV CSp-3 | 641 | U.S. Pat. No. 8,734,809 SEQ ID NO: 49 |
| AAV CSp-4 | 642 | U.S. Pat. No. 8,734,809 SEQ ID NO: 50 |
| AAV CSp-6 | 643 | U.S. Pat. No. 8,734,809 SEQ ID NO: 51 |
| AAV CSp-7 | 644 | U.S. Pat. No. 8,734,809 SEQ ID NO: 52 |
| AAV CSp-8 | 645 | U.S. Pat. No. 8,734,809 SEQ ID NO: 53 |
| AAV CSp-9 | 646 | U.S. Pat. No. 8,734,809 SEQ ID NO: 54 |
| AAV CHt-2 | 647 | U.S. Pat. No. 8,734,809 SEQ ID NO: 55 |
| AAV CHt-3 | 648 | U.S. Pat. No. 8,734,809 SEQ ID NO: 56 |
| AAV CKd-1 | 649 | U.S. Pat. No. 8,734,809 SEQ ID NO: 57 |
| AAV CKd-10 | 650 | U.S. Pat. No. 8,734,809 SEQ ID NO: 58 |
| AAV CKd-2 | 651 | U.S. Pat. No. 8,734,809 SEQ ID NO: 59 |
| AAV CKd-3 | 652 | U.S. Pat. No. 8,734,809 SEQ ID NO: 60 |
| AAV CKd-4 | 653 | U.S. Pat. No. 8,734,809 SEQ ID NO: 61 |
| AAV CKd-6 | 654 | U.S. Pat. No. 8,734,809 SEQ ID NO: 62 |
| AAV CKd-7 | 655 | U.S. Pat. No. 8,734,809 SEQ ID NO: 63 |
| AAV CKd-8 | 656 | U.S. Pat. No. 8,734,809 SEQ ID NO: 64 |
| AAV CLv-1 | 657 | U.S. Pat. No. 8,734,809 SEQ ID NO: 65 |
| AAV CLv-12 | 658 | U.S. Pat. No. 8,734,809 SEQ ID NO: 66 |
| AAV CLv-13 | 659 | U.S. Pat. No. 8,734,809 SEQ ID NO: 67 |
| AAV CLv-2 | 660 | U.S. Pat. No. 8,734,809 SEQ ID NO: 68 |
| AAV CLv-3 | 661 | U.S. Pat. No. 8,734,809 SEQ ID NO: 69 |
| AAV CLv-4 | 662 | U.S. Pat. No. 8,734,809 SEQ ID NO: 70 |
| AAV CLv-6 | 663 | U.S. Pat. No. 8,734,809 SEQ ID NO: 71 |
| AAV CLv-8 | 664 | U.S. Pat. No. 8,734,809 SEQ ID NO: 72 |
| AAV CKd-B1 | 665 | U.S. Pat. No. 8,734,809 SEQ ID NO: 73 |
| AAV CKd-B2 | 666 | U.S. Pat. No. 8,734,809 SEQ ID NO: 74 |
| AAV CKd-B3 | 667 | U.S. Pat. No. 8,734,809 SEQ ID NO: 75 |
| AAV CKd-B4 | 668 | U.S. Pat. No. 8,734,809 SEQ ID NO: 76 |
| AAV CKd-B5 | 669 | U.S. Pat. No. 8,734,809 SEQ ID NO: 77 |
| AAV CKd-B6 | 670 | U.S. Pat. No. 8,734,809 SEQ ID NO: 78 |
| AAV CKd-B7 | 671 | U.S. Pat. No. 8,734,809 SEQ ID NO: 79 |
| AAV CKd-B8 | 672 | U.S. Pat. No. 8,734,809 SEQ ID NO: 80 |
| AAV CKd-H1 | 673 | U.S. Pat. No. 8,734,809 SEQ ID NO: 81 |
| AAV CKd-H2 | 674 | U.S. Pat. No. 8,734,809 SEQ ID NO: 82 |
| AAV CKd-H3 | 675 | U.S. Pat. No. 8,734,809 SEQ ID NO: 83 |
| AAV CKd-H4 | 676 | U.S. Pat. No. 8,734,809 SEQ ID NO: 84 |
| AAV CKd-H5 | 677 | U.S. Pat. No. 8,734,809 SEQ ID NO: 85 |
| AAV CKd-H6 | 678 | U.S. Pat. No. 8,734,809 SEQ ID NO: 77 |
| AAV CHt-1 | 679 | U.S. Pat. No. 8,734,809 SEQ ID NO: 86 |
| AAV CLv1-1 | 680 | U.S. Pat. No. 8,734,809 SEQ ID NO: 171 |
| AAV CLv1-2 | 681 | U.S. Pat. No. 8,734,809 SEQ ID NO: 172 |
| AAV CLv1-3 | 682 | U.S. Pat. No. 8,734,809 SEQ ID NO: 173 |
| AAV CLv1-4 | 683 | U.S. Pat. No. 8,734,809 SEQ ID NO: 174 |
| AAV Clv1-7 | 684 | U.S. Pat. No. 8,734,809 SEQ ID NO: 175 |
| AAV Clv1-8 | 685 | U.S. Pat. No. 8,734,809 SEQ ID NO: 176 |
| AAV Clv1-9 | 686 | U.S. Pat. No. 8,734,809 SEQ ID NO: 177 |
| AAV Clv1-10 | 687 | U.S. Pat. No. 8,734,809 SEQ ID NO: 178 |
| AAV.VR-355 | 688 | U.S. Pat. No. 8,734,809 SEQ ID NO: 181 |
| AAV.hu.48R3 | 689 | U.S. Pat. No. 8,734,809 SEQ ID NO: 183 |
| AAV CBr-E1 | 690 | U.S. Pat. No. 8,734,809 SEQ ID NO: 87 |
| AAV CBr-E2 | 691 | U.S. Pat. No. 8,734,809 SEQ ID NO: 88 |
| AAV CBr-E3 | 692 | U.S. Pat. No. 8,734,809 SEQ ID NO: 89 |
| AAV CBr-E4 | 693 | U.S. Pat. No. 8,734,809 SEQ ID NO: 90 |
| AAV CBr-E5 | 694 | U.S. Pat. No. 8,734,809 SEQ ID NO: 91 |
| AAV CBr-e5 | 695 | U.S. Pat. No. 8,734,809 SEQ ID NO: 92 |
| AAV CBr-E6 | 696 | U.S. Pat. No. 8,734,809 SEQ ID NO: 93 |
| AAV CBr-E7 | 697 | U.S. Pat. No. 8,734,809 SEQ ID NO: 94 |
| AAV CBr-E8 | 698 | U.S. Pat. No. 8,734,809 SEQ ID NO: 95 |
| AAV CLv-D1 | 699 | U.S. Pat. No. 8,734,809 SEQ ID NO: 96 |
| AAV CLv-D2 | 700 | U.S. Pat. No. 8,734,809 SEQ ID NO: 97 |
| AAV CLv-D3 | 701 | U.S. Pat. No. 8,734,809 SEQ ID NO: 98 |
| AAV CLv-D4 | 702 | U.S. Pat. No. 8,734,809 SEQ ID NO: 99 |
| AAV CLv-D5 | 703 | U.S. Pat. No. 8,734,809 SEQ ID NO: 100 |
| AAV CLv-D6 | 704 | U.S. Pat. No. 8,734,809 SEQ ID NO: 101 |
| AAV CLv-D7 | 705 | U.S. Pat. No. 8,734,809 SEQ ID NO: 102 |
| AAV CLv-D8 | 706 | U.S. Pat. No. 8,734,809 SEQ ID NO: 103 |
| AAV CLv-E1 | 707 | U.S. Pat. No. 8,734,809 SEQ ID NO: 87 |
| AAV CLv-R1 | 708 | U.S. Pat. No. 8,734,809 SEQ ID NO: 104 |
| AAV CLv-R2 | 709 | U.S. Pat. No. 8,734,809 SEQ ID NO: 105 |
| AAV CLv-R3 | 710 | U.S. Pat. No. 8,734,809 SEQ ID NO: 106 |
| AAV CLv-R4 | 711 | U.S. Pat. No. 8,734,809 SEQ ID NO: 107 |
| AAV CLv-R5 | 712 | U.S. Pat. No. 8,734,809 SEQ ID NO: 108 |
| AAV CLv-R6 | 713 | U.S. Pat. No. 8,734,809 SEQ ID NO: 109 |
| AAV CLv-R7 | 714 | U.S. Pat. No. 8,734,809 SEQ ID NO: 110 |
| AAV CLv-R8 | 715 | U.S. Pat. No. 8,734,809 SEQ ID NO: 111 |
| AAV CLv-R9 | 716 | U.S. Pat. No. 8,734,809 SEQ ID NO: 112 |
| AAV CLg-F1 | 717 | U.S. Pat. No. 8,734,809 SEQ ID NO: 113 |
| AAV CLg-F2 | 718 | U.S. Pat. No. 8,734,809 SEQ ID NO: 114 |
| AAV CLg-F3 | 719 | U.S. Pat. No. 8,734,809 SEQ ID NO: 115 |
| AAV CLg-F4 | 720 | U.S. Pat. No. 8,734,809 SEQ ID NO: 116 |
| AAV CLg-F5 | 721 | U.S. Pat. No. 8,734,809 SEQ ID NO: 117 |
| AAV CLg-F6 | 722 | U.S. Pat. No. 8,734,809 SEQ ID NO: 117 |
| AAV CLg-F7 | 723 | U.S. Pat. No. 8,734,809 SEQ ID NO: 118 |
| AAV CLg-F8 | 724 | U.S. Pat. No. 8,734,809 SEQ ID NO: 117 |
| AAV CSp-1 | 725 | U.S. Pat. No. 8,734,809 SEQ ID NO: 119 |
| AAV CSp-10 | 726 | U.S. Pat. No. 8,734,809 SEQ ID NO: 120 |
| AAV CSp-11 | 727 | U.S. Pat. No. 8,734,809 SEQ ID NO: 121 |
| AAV CSp-2 | 728 | U.S. Pat. No. 8,734,809 SEQ ID NO: 122 |
| AAV CSp-3 | 729 | U.S. Pat. No. 8,734,809 SEQ ID NO: 123 |
| AAV CSp-4 | 730 | U.S. Pat. No. 8,734,809 SEQ ID NO: 124 |
| AAV CSp-6 | 731 | U.S. Pat. No. 8,734,809 SEQ ID NO: 125 |
| AAV CSp-7 | 732 | U.S. Pat. No. 8,734,809 SEQ ID NO: 126 |
| AAV CSp-8 | 733 | U.S. Pat. No. 8,734,809 SEQ ID NO: 127 |
| AAV CSp-9 | 734 | U.S. Pat. No. 8,734,809 SEQ ID NO: 128 |
| AAV CHt-2 | 735 | U.S. Pat. No. 8,734,809 SEQ ID NO: 129 |
| AAV CHt-3 | 736 | U.S. Pat. No. 8,734,809 SEQ ID NO: 130 |
| AAV CKd-1 | 737 | U.S. Pat. No. 8,734,809 SEQ ID NO: 131 |
| AAV CKd-10 | 738 | U.S. Pat. No. 8,734,809 SEQ ID NO: 132 |
| AAV CKd-2 | 739 | U.S. Pat. No. 8,734,809 SEQ ID NO: 133 |
| AAV CKd-3 | 740 | U.S. Pat. No. 8,734,809 SEQ ID NO: 134 |
| AAV CKd-4 | 741 | U.S. Pat. No. 8,734,809 SEQ ID NO: 135 |
| AAV CKd-6 | 742 | U.S. Pat. No. 8,734,809 SEQ ID NO: 136 |
| AAV CKd-7 | 743 | U.S. Pat. No. 8,734,809 SEQ ID NO: 137 |
| AAV CKd-8 | 744 | U.S. Pat. No. 8,734,809 SEQ ID NO: 138 |
| AAV CLv-1 | 745 | U.S. Pat. No. 8,734,809 SEQ ID NO: 139 |
| AAV CLv-12 | 746 | U.S. Pat. No. 8,734,809 SEQ ID NO: 140 |
| AAV CLv-13 | 747 | U.S. Pat. No. 8,734,809 SEQ ID NO: 141 |
| AAV CLv-2 | 748 | U.S. Pat. No. 8,734,809 SEQ ID NO: 142 |
| AAV CLv-3 | 749 | U.S. Pat. No. 8,734,809 SEQ ID NO: 143 |
| AAV CLv-4 | 750 | U.S. Pat. No. 8,734,809 SEQ ID NO: 144 |
| AAV CLv-6 | 751 | U.S. Pat. No. 8,734,809 SEQ ID NO: 145 |
| AAV CLv-8 | 752 | U.S. Pat. No. 8,734,809 SEQ ID NO: 146 |
| AAV CKd-B1 | 753 | U.S. Pat. No. 8,734,809 SEQ ID NO: 147 |
| AAV CKd-B2 | 754 | U.S. Pat. No. 8,734,809 SEQ ID NO: 148 |
| AAV CKd-B3 | 755 | U.S. Pat. No. 8,734,809 SEQ ID NO: 149 |
| AAV CKd-B4 | 756 | U.S. Pat. No. 8,734,809 SEQ ID NO: 150 |
| AAV CKd-B5 | 757 | U.S. Pat. No. 8,734,809 SEQ ID NO: 151 |
| AAV CKd-B6 | 758 | U.S. Pat. No. 8,734,809 SEQ ID NO: 152 |
| AAV CKd-B7 | 759 | U.S. Pat. No. 8,734,809 SEQ ID NO: 153 |
| AAV CKd-B8 | 760 | U.S. Pat. No. 8,734,809 SEQ ID NO: 154 |
| AAV CKd-H1 | 761 | U.S. Pat. No. 8,734,809 SEQ ID NO: 155 |
| AAV CKd-H2 | 762 | U.S. Pat. No. 8,734,809 SEQ ID NO: 156 |
| AAV CKd-H3 | 763 | U.S. Pat. No. 8,734,809 SEQ ID NO: 157 |
| AAV CKd-H4 | 764 | U.S. Pat. No. 8,734,809 SEQ ID NO: 158 |
| AAV CKd-H5 | 765 | U.S. Pat. No. 8,734,809 SEQ ID NO: 159 |
| AAV CKd-H6 | 766 | U.S. Pat. No. 8,734,809 SEQ ID NO: 151 |
| AAV CHt-1 | 767 | U.S. Pat. No. 8,734,809 SEQ ID NO: 160 |
| AAV CHt-P2 | 768 | WO2016065001 SEQ ID NO: 1 |
| AAV CHt-P5 | 769 | WO2016065001 SEQ ID NO: 2 |
| AAV CHt-P9 | 770 | WO2016065001 SEQ ID NO: 3 |
| AAV CBr-7.1 | 771 | WO2016065001 SEQ ID NO: 4 |
| AAV CBr-7.2 | 772 | WO2016065001 SEQ ID NO: 5 |
| AAV CBr-7.3 | 773 | WO2016065001 SEQ ID NO: 6 |
| AAV CBr-7.4 | 774 | WO2016065001 SEQ ID NO: 7 |
| AAV CBr-7.5 | 775 | WO2016065001 SEQ ID NO: 8 |
| AAV CBr-7.7 | 776 | WO2016065001 SEQ ID NO: 9 |
| AAV CBr-7.8 | 777 | WO2016065001 SEQ ID NO: 10 |
| AAV CBr-7.10 | 778 | WO2016065001 SEQ ID NO: 11 |
| AAV CKd-N3 | 779 | WO2016065001 SEQ ID NO: 12 |
| AAV CKd-N4 | 780 | WO2016065001 SEQ ID NO: 13 |
| AAV CKd-N9 | 781 | WO2016065001 SEQ ID NO: 14 |
| AAV CLv-L4 | 782 | WO2016065001 SEQ ID NO: 15 |
| AAV CLv-L5 | 783 | WO2016065001 SEQ ID NO: 16 |
| AAV CLv-L6 | 784 | WO2016065001 SEQ ID NO: 17 |
| AAV CLv-K1 | 785 | WO2016065001 SEQ ID NO: 18 |
| AAV CLv-K3 | 786 | WO2016065001 SEQ ID NO: 19 |
| AAV CLv-K6 | 787 | WO2016065001 SEQ ID NO: 20 |
| AAV CLv-M1 | 788 | WO2016065001 SEQ ID NO: 21 |
| AAV CLv-M11 | 789 | WO2016065001 SEQ ID NO: 22 |
| AAV CLv-M2 | 790 | WG2016065001 SEQ ID NO: 23 |
| AAV CLv-M5 | 791 | WO2016065001 SEQ ID NO: 24 |
| AAV CLv-M6 | 792 | WO2016065001 SEQ ID NO: 25 |
| AAV CLv-M7 | 793 | WO2016065001 SEQ ID NO: 26 |
| AAV CLv-M8 | 794 | WO2016065001 SEQ ID NO: 27 |
| AAV CLv-M9 | 795 | WO2016065001 SEQ ID NO: 28 |
| AAV CHt-P1 | 796 | WO2016065001 SEQ ID NO: 29 |
| AAV CHt-P6 | 797 | WO2016065001 SEQ ID NO: 30 |
| AAV CHt-P8 | 798 | WO2016065001 SEQ ID NO: 31 |
| AAV CHt-6.1 | 799 | WO2016065001 SEQ ID NO: 32 |
| AAV CHt-6.10 | 800 | WO2016065001 SEQ ID NO: 33 |
| AAV CHt-6.5 | 801 | WO2016065001 SEQ ID NO: 34 |
| AAV CHt-6.6 | 802 | WO2016065001 SEQ ID NO: 35 |
| AAV CHt-6.7 | 803 | WO2016065001 SEQ ID NO: 36 |
| AAV CHt-6.8 | 804 | WO2016065001 SEQ ID NO: 37 |
| AAV CSp-8.10 | 805 | WO2016065001 SEQ ID NO: 38 |
| AAV CSp-8.2 | 806 | WO2016065001 SEQ ID NO: 39 |
| AAV CSp-8.4 | 807 | WO2016065001 SEQ ID NO: 40 |
| AAV CSp-8.5 | 808 | WO2016065001 SEQ ID NO: 41 |
| AAV CSp-8.6 | 809 | WO2016065001 SEQ ID NO: 42 |
| AAV CSp-8.7 | 810 | WO2016065001 SEQ ID NO: 43 |
| AAV CSp-8.8 | 811 | WO2016065001 SEQ ID NO: 44 |
| AAV CSp-8.9 | 812 | WO2016065001 SEQ ID NO: 45 |
| AAV CBr-B7.3 | 813 | WO2016065001 SEQ ID NO: 46 |
| AAV CBr-B7.4 | 814 | WO2016065001 SEQ ID NO: 47 |
| AAV3B | 815 | WO2016065001 SEQ ID NO: 48 |
| AAV4 | 816 | WO2016065001 SEQ ID NO: 49 |
| AAV5 | 817 | WO2016065001 SEQ ID NO: 50 |
| AAV CHt-P2 | 818 | WO2016065001 SEQ ID NO: 51 |
| AAV CHt-P5 | 819 | WO2016065001 SEQ ID NO: 52 |
| AAV CHt-P9 | 820 | WO2016065001 SEQ ID NO: 53 |
| AAV CBr-7.1 | 821 | WO2016065001 SEQ ID NO: 54 |
| AAV CBr-7.2 | 822 | WO2016065001 SEQ ID NO: 55 |
| AAV CBr-7.3 | 823 | WO2016065001 SEQ ID NO: 56 |
| AAV CBr-7.4 | 824 | WO2016065001 SEQ ID NO: 57 |
| AAV CBr-7.5 | 825 | WO2016065001 SEQ ID NO: 58 |
| AAV CBr-7.7 | 826 | WO2016065001 SEQ ID NO: 59 |
| AAV CBr-7.8 | 827 | WO2016065001 SEQ ID NO: 60 |
| AAV CBr-7.10 | 828 | WO2016065001 SEQ ID NO: 61 |
| AAV CKd-N3 | 829 | WO2016065001 SEQ ID NO: 62 |
| AAV CKd-N4 | 830 | WO2016065001 SEQ ID NO: 63 |
| AAV CKd-N9 | 831 | WO2016065001 SEQ ID NO: 64 |
| AAV CLv-L4 | 832 | WO2016065001 SEQ ID NO: 65 |
| AAV CLv-L5 | 833 | WO2016065001 SEQ ID NO: 66 |
| AAV CLv-L6 | 834 | WO2016065001 SEQ ID NO: 67 |
| AAV CLv-K1 | 835 | WO2016065001 SEQ ID NO: 68 |
| AAV CLv-K3 | 836 | WO2016065001 SEQ ID NO: 69 |
| AAV CLv-K6 | 837 | WO2016065001 SEQ ID NO: 70 |
| AAV CLv-M1 | 838 | WO2016065001 SEQ ID NO: 71 |
| AAV CLv-M11 | 839 | WO2016065001 SEQ ID NO: 72 |
| AAV CLv-M2 | 840 | WO2016065001 SEQ ID NO: 73 |
| AAV CLv-M5 | 841 | WO2016065001 SEQ ID NO: 74 |
| AAV CLv-M6 | 842 | WO2016065001 SEQ ID NO: 75 |
| AAV CLv-M7 | 843 | WO2016065001 SEQ ID NO: 76 |
| AAV CLv-M8 | 844 | WO2016065001 SEQ ID NO: 77 |
| AAV CLv-M9 | 845 | WO2016065001 SEQ ID NO: 78 |
| AAV CHt-P1 | 846 | WO2016065001 SEQ ID NO: 79 |
| AAV CHt-P6 | 847 | WO2016065001 SEQ ID NO: 80 |
| AAV CHt-P8 | 848 | WO2016065001 SEQ ID NO: 81 |
| AAV CHt-6.1 | 849 | WO2016065001 SEQ ID NO: 82 |
| AAV CHt-6.10 | 850 | WO2016065001 SEQ ID NO: 83 |
| AAV CHt-6.5 | 851 | WO2016065001 SEQ ID NO: 84 |
| AAV CHt-6.6 | 852 | WO2016065001 SEQ ID NO: 85 |
| AAV CHt-6.7 | 853 | WO2016065001 SEQ ID NO: 86 |
| AAV CHt-6.8 | 854 | WO2016065001 SEQ ID NO: 87 |
| AAV CSp-8.10 | 855 | WO2016065001 SEQ ID NO: 88 |
| AAV CSp-8.2 | 856 | WO2016065001 SEQ ID NO: 89 |
| AAV CSp-8.4 | 857 | WO2016065001 SEQ ID NO: 90 |
| AAV CSp-8.5 | 858 | WO2016065001 SEQ ID NO: 91 |
| AAV CSp-8.6 | 859 | WO2016065001 SEQ ID NO: 92 |
| AAV CSp-8.7 | 860 | WO2016065001 SEQ ID NO: 93 |
| AAV CSp-8.8 | 861 | WO2016065001 SEQ ID NO: 94 |
| AAV CSp-8.9 | 862 | WO2016065001 SEQ ID NO: 95 |
| AAV CBr-B7.3 | 863 | WO2016065001 SEQ ID NO: 96 |
| AAV CBr-B7.4 | 864 | WO2016065001 SEQ ID NO: 97 |
| AAV3B | 865 | WO2016065001 SEQ ID NO: 98 |
| AAV4 | 866 | WO2016065001 SEQ ID NO: 99 |
| AAV5 | 867 | WO2016065001 SEQ ID NO: 100 |
| AAVPHP.B | 868 | WO2015038958 SEQ ID NO: 8 |
| or G2B-26 | | and 13; GenBankALU85156.1 |
| AAVPHP.B | 869 | WO2015038958 SEQ ID NO: 9 |
| AAVG2B-13 | 870 | WO2015038958 SEQ ID NO: 12 |
| AAVTH1.1-32 | 871 | WO2015038958 SEQ ID NO: 14 |
| AAVTH1.1-35 | 872 | WO2015038958 SEQ ID NO: 15 |
| PHP.N/ | 1859 | WO2017100671 SEQ ID NO: 46 |
| PHP.B-DGT | | |
| PHP.S/G2A12 | 1860 | WO2017100671 SEQ ID NO: 47 |
| AAV9/ | 1861 | WO2017100671 SEQ ID NO: 45 |
| hu.14K449R | | |
| GPV | 1862 | U.S. Pat. No. 9,624,274B2 SEQ ID NO: 192 |
| B19 | 1863 | U.S. Pat. No. 9,624,274B2 SEQ ID NO: 193 |
| MVM | 1864 | U.S. Pat. No. 9,624,274B2 SEQ ID NO: 194 |
| CPV | 1865 | U.S. Pat. No. 9,624,274B2 SEQ ID NO: 195 |
| CPV | 1866 | U.S. Pat. No. 9,624,274B2 SEQ ID NO: 196 |
| AAV6 | 1867 | U.S. Pat. No. 9,546,112B2 SEQ ID NO: 5 |
| AAV6 | 1868 | U.S. Pat. No. 9,457,103B2 SEQ ID NO: 1 |
| AAV2 | 1869 | U.S. Pat. No. 9,457,103B2 SEQ ID NO: 2 |
| ShH10 | 1870 | U.S. Pat. No. 9,457,103B2 SEQ ID NO: 3 |
| ShH13 | 1871 | U.S. Pat. No. 9,457,103B2 SEQ ID NO: 4 |
| ShH10 | 1872 | U.S. Pat. No. 9,457,103B2 SEQ ID NO: 5 |
| ShH10 | 1873 | U.S. Pat. No. 9,457,103B2 SEQ ID NO: 6 |
| ShH10 | 1874 | U.S. Pat. No. 9,457,103B2 SEQ ID NO: 7 |
| ShH10 | 1875 | U.S. Pat. No. 9,457,103B2 SEQ ID NO: 8 |
| ShH10 | 1876 | U.S. Pat. No. 9,457,103B2 SEQ ID NO: 9 |
| rh74 | 1877 | U.S. Pat. No. 9,4349,28B2 SEQ ID NO: 1, |
| | | US2015023924A1 SEQ ID NO: 2 |
| rh74 | 1878 | U.S. Pat. No. 9,434,928B2 SEQ ID NO: 2, |
| | | US2015023924A1 SEQ ID NO: 1 |
| AAV8 | 1879 | U.S. Pat. No. 9,434,928B2 SEQ ID NO: 4 |
| rh74 | 1880 | U.S. Pat. No. 9,434,928B2 SEQ ID NO: 5 |
| rh74 | 1881 | US2015023924A1 SEQ ID NO: 5, |
| (RHM4-1) | | US20160375110A1 SEQ ID NO: 4 |
| rh74 | 1882 | US2015023924A1 SEQ ID NO: 6, |
| (RHM15-1) | | US20160375110A1 SEQ ID NO: 5 |
| rh74 | 1883 | US2015023924A1 SEQ ID NO: 7, |
| (RHM15-2) | | US20160375110A1 SEQ ID NO: 6 |
| rh74 | 1884 | US2015023924A1 SEQ ID NO: 8, |
| (RHM15-3/ | | US20160375110A1 SEQ ID NO: 7 |
| RHM15-5) | | |
| rh74 | 1885 | US2015023924A1 SEQ ID NO: 9, |
| (RHM15-4) | | US20160375110A1 SEQ ID NO: 8 |
| rh74 | 1886 | US2015023924A1 SEQ ID NO: 10, |
| (RHM15-6) | | US20160375110A1 SEQ ID NO: 9 |
| rh74 | 1887 | US2015023924A1 SEQ ID NO: 11 |
| (RHM4-1) | | |
| rh74 | 1888 | US2015023924A1 SEQ ID NO: 12 |
| (RHM15-1) | | |
| rh74 | 1889 | US2015023924A1 SEQ ID NO: 13 |
| (RHM15-2) | | |
| rh74 | 1890 | US2015023924A1 SEQ ID NO: 14 |
| (RHM15-3/ | | |
| RHM15-5) | | |
| rh74 | 1891 | US2015023924A1 SEQ ID NO: 15 |
| (RHM15-4) | | |
| rh74 | 1892 | US2015023924A1 SEQ ID NO: 16 |
| (RHM15-6) | | |
| AAV2 | 1893 | US20160175389A1 SEQ ID NO: 9 |
| (comprising | | |
| lung specific | | |
| polypeptide) | | |
| AAV2 | 1894 | US20160175389A1 SEQ ID NO: 10 |
| (comprising | | |
| lung specific | | |
| polypeptide) | | |
| Anc80 | 1895 | US20170051257A1 SEQ ID NO: 1 |
| Anc80 | 1896 | US20170051257A1 SEQ ID NO: 2 |
| Anc81 | 1897 | US20170051257A1 SEQ ID NO: 3 |
| Anc80 | 1898 | US20170051257A1 SEQ ID NO: 4 |
| Anc82 | 1899 | US20170051257A1 SEQ ID NO: 5 |
| Anc82 | 1900 | US20170051257A1 SEQ ID NO: 6 |
| Anc83 | 1901 | US20170051257A1 SEQ ID NO: 7 |
| Anc83 | 1902 | US20170051257A1 SEQ ID NO: 8 |
| Anc84 | 1903 | US20170051257A1 SEQ ID NO: 9 |
| Anc84 | 1904 | US20170051257A1 SEQ ID NO: 10 |
| Anc94 | 1905 | US20170051257A1 SEQ ID NO: 11 |
| Anc94 | 1906 | US20170051257A1 SEQ ID NO: 12 |
| Anc113 | 1907 | US20170051257A1 SEQ ID NO: 13 |
| Anc113 | 1908 | US20170051257A1 SEQ ID NO: 14 |
| Anc126 | 1909 | US20170051257A1 SEQ ID NO: 15 |
| Anc126 | 1910 | US20170051257A1 SEQ ID NO: 16 |
| Anc127 | 1911 | US20170051257A1 SEQ ID NO: 17 |
| Anc127 | 1912 | US20170051257A1 SEQ ID NO: 18 |
| Anc80L27 | 1913 | US20170051257A1 SEQ ID NO: 19 |
| Anc80L59 | 1914 | US20170051257A1 SEQ ID NO: 20 |
| Anc80L60 | 1915 | US20170051257A1 SEQ ID NO: 21 |
| Anc80L62 | 1916 | US20170051257A1 SEQ ID NO: 22 |
| Anc80L65 | 1917 | US20170051257A1 SEQ ID NO: 23 |
| Anc80L33 | 1918 | US20170051257A1 SEQ ID NO: 24 |
| Anc80L36 | 1919 | US20170051257A1 SEQ ID NO: 25 |
| Anc80L44 | 1920 | US20170051257A1 SEQ ID NO: 26 |
| Anc80L1 | 1921 | US20170051257A1 SEQ ID NO: 35 |
| Anc80L1 | 1922 | US20170051257A1 SEQ ID NO: 36 |
| AAV-X1 | 1923 | U.S. Pat. No. 8,283,151B2 SEQ ID NO: 11 |
| AAV-X1b | 1924 | U.S. Pat. No. 8,283,151B2 SEQ ID NO: 12 |
| AAV-X5 | 1925 | U.S. Pat. No. 8,283,151B2 SEQ ID NO: 13 |
| AAV-X19 | 1926 | U.S. Pat. No. 8,283,151B2 SEQ ID NO: 14 |
| AAV-X22 | 1927 | U.S. Pat. No. 8,283,151B2 SEQ ID NO: 15 |
| AAV-X22 | 1928 | U.S. Pat. No. 8,283,151B2 SEQ ID NO: 16 |
| AAV-X23 | 1929 | U.S. Pat. No. 8,283,151B2 SEQ ID NO: 17 |
| AAV-X24 | 1930 | U.S. Pat. No. 8,283,151B2 SEQ ID NO: 18 |
| AAV-X25 | 1931 | U.S. Pat. No. 8,283,151B2 SEQ ID NO: 19 |
| AAV-X26 | 1932 | U.S. Pat. No. 8,283,151B2 SEQ ID NO: 20 |
| AAV-X1 | 1933 | U.S. Pat. No. 8,283,151B2 SEQ ID NO: 21 |
| AAV-X1b | 1934 | U.S. Pat. No. 8,283,151B2 SEQ ID NO: 22 |
| AAV-X5 | 1935 | U.S. Pat. No. 8,283,151B2 SEQ ID NO: 23 |
| AAV-X19 | 1936 | U.S. Pat. No. 8,283,151B2 SEQ ID NO: 24 |
| AAV-X21 | 1937 | U.S. Pat. No. 8,283,151B2 SEQ ID NO: 25 |
| AAV-X22 | 1938 | U.S. Pat. No. 8,283,151B2 SEQ ID NO: 26 |
| AAV-X23 | 1939 | U.S. Pat. No. 8,283,151B2 SEQ ID NO: 27 |
| AAV-X24 | 1940 | U.S. Pat. No. 8,283,151B2 SEQ ID NO: 28 |
| AAV-X25 | 1941 | U.S. Pat. No. 8,283,151B2 SEQ ID NO: 29 |
| AAV-X26 | 1942 | U.S. Pat. No. 8,283,151B2 SEQ ID NO: 30 |
| AAVrh8 | 1943 | WO2016054554A1 SEQ ID NO: 8 |
| AAVrh8VP2FC5 | 1944 | WO2016054554A1 SEQ ID NO: 9 |
| AAVrh8VP2FC44 | 1945 | WO2016054554A1 SEQ ID NO: 30 |
| AAVrh8VP2ApoB100 | 1946 | WO2016054554A1 SEQ ID NO: 11 |
| AAVrh8VP2RVG | 1947 | WO2016054554A1 SEQ ID NO: 12 |
| AAVrh8VP2 | 1948 | WO2016054554A1 SEQ ID NO: 13 |
| Angiopep-2VP2 | | |
| AAV9.47VP1.3 | 1949 | WO2016054554A1 SEQ ID NO: 14 |
| AAV9.47VP2ICAMg3 | 1950 | WO2016054554A1 SEQ ID NO: 15 |
| AAV9.47VP2RVG | 1951 | WO2016054554A1 SEQ ID NO: 16 |
| AAV9.47VP2Angiopep-2 | 1952 | WO2016054554A1 SEQ ID NO: 17 |
| AAV9.47VP2A- | 1953 | WO2016054554A1 SEQ ID NO: 18 |
| string | | |
| AAVrh8VP2FC5 | 1954 | WO2016054554A1 SEQ ID NO: 19 |
| VP2 | | |
| AAVrh8VP2FC44 | 1955 | WO2016054554A1 SEQ ID NO: 20 |
| VP2 | | |
| AAVrb8VP2ApoB100 | 1956 | WO2016054554A1 SEQ ID NO: 21 |
| VP2 | | |
| AAVrh8VP2RVG | 1957 | WO2016054554A1 SEQ ID NO: 22 |
| VP2 | | |
| AAVrh8VP2 | 1958 | WO2016054554A1 SEQ ID NO: 23 |
| Angiopep-2VP2 | | |
| AAV9.47VP2ICAMg3 | 1959 | WO2016054554A1 SEQ ID NO: 24 |
| VP2 | | |
| AAV9.47VP2RVG | 1960 | WO2016054554A1 SEQ ID NO: 25 |
| VP2 | | |
| AAV9.47VP2Angiopep- | 1961 | WO2016054554A1 SEQ ID NO: 26 |
| 2 VP2 | | |
| AAV9.47VP2A- | 1962 | WO2016054554A1 SEQ ID NO: 27 |
| string VP2 | | |
| rAAV-B1 | 1963 | WO2016054557A1 SEQ ID NO: 1 |
| rAAV-B2 | 1964 | WO2016054557A1 SEQ ID NO: 2 |
| rAAV-B3 | 1965 | WO2016054557A1 SEQ ID NO: 3 |
| rAAV-B4 | 1966 | WO2016054557A1 SEQ ID NO: 4 |
| rAAV-B1 | 1967 | WO2016054557A1 SEQ ID NO: 5 |
| rAAV-B2 | 1968 | WO2016054557A1 SEQ ID NO: 6 |
| rAAV-B3 | 1969 | WO2016054557A1 SEQ ID NO: 7 |
| rAAV-B4 | 1970 | WO2016054557A1 SEQ ID NO: 8 |
| rAAV-L1 | 1971 | WO2016054557A1 SEQ ID NO: 9 |
| rAAV-L2 | 1972 | WO2016054557A1 SEQ ID NO: 10 |
| rAAV-L3 | 1973 | WO2016054557A1 SEQ ID NO: 11 |
| rAAV-L4 | 1974 | WO2016054557A1 SEQ ID NO: 12 |
| rAAV-L1 | 1975 | WO2016054557A1 SEQ ID NO: 13 |
| rAAV-L2 | 1976 | WO2016054557A1 SEQ ID NO: 14 |
| rAAV-L3 | 1977 | WO2016054557A1 SEQ ID NO: 15 |
| rAAV-L4 | 1978 | WO2016054557A1 SEQ ID NO: 16 |
| AAV9 | 1979 | WO2016073739A1 SEQ ID NO: 3 |
| rAAV | 1980 | WO2016081811A1 SEQ ID NO: 1 |
| rAAV | 1981 | WO2016081811A1 SEQ ID NO: 2 |
| rAAV | 1982 | WO2016081811A1 SEQ ID NO: 3 |
| rAAV | 1983 | WO2016081811A1 SEQ ID NO: 4 |
| rAAV | 1984 | WO2016081811A1 SEQ ID NO: 5 |
| rAAV | 1985 | WO2016081811A1 SEQ ID NO: 6 |
| rAAV | 1986 | WO2016081811A1 SEQ ID NO: 7 |
| rAAV | 1987 | WO2016081811A1 SEQ ID NO: 8 |
| rAAV | 1988 | WO2016081811A1 SEQ ID NO: 9 |
| rAAV | 1989 | WO2016081811A1 SEQ ID NO: 10 |
| rAAV | 1990 | WO2016081811A1 SEQ ID NO: 11 |
| rAAV | 1991 | WO2016081811A1 SEQ ID NO: 12 |
| rAAV | 1992 | WO2016081811A1 SEQ ID NO: 13 |
| rAAV | 1997 | WO2016081811A1 SEQ ID NO: 14 |
| rAAV | 1994 | WO2016081811A1 SEQ ID NO: 15 |
| rAAV | 1995 | WO2016081811A1 SEQ ID NO: 16 |
| rAAV | 1996 | WO2016081811A1 SEQ ID NO: 17 |
| rAAV | 1997 | WO2016081811A1 SEQ ID NO: 18 |
| rAAV | 1998 | WO2016081811A1 SEQ ID NO: 19 |
| rAAV | 1999 | WO2016081811A1 SEQ ID NO: 20 |
| rAAV | 2000 | WO2016081811A1 SEQ ID NO: 21 |
| rAAV | 2001 | WO2016081811A1 SEQ ID NO: 22 |
| rAAV | 2002 | WO2016081811A1 SEQ ID NO: 23 |
| rAAV | 2003 | WO2016081811A1 SEQ ID NO: 24 |
| rAAV | 2004 | WO2016081811A1 SEQ ID NO: 25 |
| rAAV | 2005 | WO2016081811A1 SEQ ID NO: 26 |
| rAAV | 2006 | WO2016081811A1 SEQ ID NO: 27 |
| rAAV | 2007 | WO2016081811A1 SEQ ID NO: 28 |
| rAAV | 2008 | WO2016081811A1 SEQ ID NO: 29 |
| rAAV | 2009 | WO2016081811A1 SEQ ID NO: 30 |
| rAAV | 2010 | WO2016081811A1 SEQ ID NO: 31 |
| rAAV | 2011 | WO2016081811A1 SEQ ID NO: 32 |
| rAAV | 2012 | WO2016081811A1 SEQ ID NO: 33 |
| rAAV | 2013 | WO2016081811A1 SEQ ID NO: 34 |
| rAAV | 2014 | WO2016081811A1 SEQ ID NO: 35 |
| rAAV | 2015 | WO2016081811A1 SEQ ID NO: 36 |
| rAAV | 2016 | WO2016081811A1 SEQ ID NO: 37 |
| rAAV | 2017 | WO2016081811A1 SEQ ID NO: 38 |
| rAAV | 2018 | WO2016081811A1 SEQ ID NO: 39 |
| rAAV | 2019 | WO2016081811A1 SEQ ID NO: 40 |
| rAAV | 2020 | WO2016081811A1 SEQ ID NO: 41 |
| rAAV | 2021 | WO2016081811A1 SEQ ID NO: 42 |
| rAAV | 2022 | WO2016081811A1 SEQ ID NO: 43 |
| rAAV | 2023 | WO2016081811A1 SEQ ID NO: 44 |
| rAAV | 2024 | WO2016081811A1 SEQ ID NO: 45 |
| rAAV | 2025 | WO2016081811A1 SEQ ID NO: 46 |
| rAAV | 2026 | WO2016081811A1 SEQ ID NO: 47 |
| rAAV | 2027 | WO2016081811A1 SEQ ID NO: 48 |
| rAAV | 2028 | WO2016081811A1 SEQ ID NO: 49 |
| rAAV | 2029 | WO2016081811A1 SEQ ID NO: 50 |
| rAAV | 2030 | WO2016081811A1 SEQ ID NO: 51 |
| rAAV | 2031 | WO2016081811A1 SEQ ID NO: 52 |
| rAAV | 2032 | WO2016081811A1 SEQ ID NO: 53 |
| rAAV | 2033 | WO2016081811A1 SEQ ID NO: 54 |
| rAAV | 2034 | WO2016081811A1 SEQ ID NO: 55 |
| rAAV | 2035 | WO2016081811A1 SEQ ID NO: 56 |
| rAAV | 2036 | WO2016081811A1 SEQ ID NO: 57 |
| rAAV | 2037 | WO2016081811A1 SEQ ID NO: 58 |
| rAAV | 2038 | WO2016081811A1 SEQ ID NO: 59 |
| rAAV | 2039 | WO2016081811A1 SEQ ID NO: 60 |
| rAAV | 2040 | WO2016081811A1 SEQ ID NO: 61 |
| rAAV | 2041 | WO2016081811A1 SEQ ID NO: 62 |
| rAAV | 2042 | WO2016081811A1 SEQ ID NO: 63 |
| rAAV | 2043 | WO2016081811A1 SEQ ID NO: 64 |
| rAAV | 2044 | WO2016081811A1 SEQ ID NO: 65 |
| rAAV | 2045 | WO2016081811A1 SEQ ID NO: 66 |
| rAAV | 2046 | WO2016081811A1 SEQ ID NO: 67 |
| rAAV | 2047 | WO2016081811A1 SEQ ID NO: 68 |
| rAAV | 2048 | WO2016081811A1 SEQ ID NO: 69 |
| rAAV | 2049 | WO2016081811A1 SEQ ID NO: 70 |
| rAAV | 2050 | WO2016081811A1 SEQ ID NO: 71 |
| rAAV | 2051 | WO2016081811A1 SEQ ID NO: 72 |
| rAAV | 2052 | WO2016081811A1 SEQ ID NO: 73 |
| rAAV | 2053 | WO2016081811A1 SEQ ID NO: 74 |
| rAAV | 2054 | WO2016081811A1 SEQ ID NO: 75 |
| rAAV | 2055 | WO2016081811A1 SEQ ID NO: 76 |
| rAAV | 2056 | WO2016081811A1 SEQ ID NO: 77 |
| rAAV | 2057 | WO2016081811A1 SEQ ID NO: 78 |
| rAAV | 2058 | WO2016081811A1 SEQ ID NO: 79 |
| rAAV | 2059 | WO2016081811A1 SEQ ID NO: 80 |
| rAAV | 2060 | WO2016081811A1 SEQ ID NO: 81 |
| rAAV | 2061 | WO2016081811A1 SEQ ID NO: 82 |
| rAAV | 2062 | WO2016081811A1 SEQ ID NO: 83 |
| rAAV | 2063 | WO2016081811A1 SEQ ID NO: 84 |
| rAAV | 2064 | WO2016081811A1 SEQ ID NO: 85 |
| rAAV | 2065 | WO2016081811A1 SEQ ID NO: 86 |
| rAAV | 2066 | WO2016081811A1 SEQ ID NO: 87 |
| rAAV | 2067 | WO2016081811A1 SEQ ID NO: 88 |
| rAAV | 2068 | WO2016081811A1 SEQ ID NO: 89 |
| rAAV | 2069 | WO2016081811A1 SEQ ID NO: 90 |
| rAAV | 2070 | WO2016081811A1 SEQ ID NO: 91 |
| rAAV | 2071 | WO2016081811A1 SEQ ID NO: 92 |
| rAAV | 2072 | WO2016081811A1 SEQ ID NO: 93 |
| rAAV | 2073 | WO2016081811A1 SEQ ID NO: 94 |
| rAAV | 2074 | WO2016081811A1 SEQ ID NO: 95 |
| rAAV | 2075 | WO2016081811A1 SEQ ID NO: 96 |
| rAAV | 2076 | WO2016081811A1 SEQ ID NO: 97 |
| rAAV | 2077 | WO2016081811A1 SEQ ID NO: 98 |
| rAAV | 2078 | WO2016081811A1 SEQ ID NO: 99 |
| rAAV | 2079 | WO2016081811A1 SEQ ID NO: 100 |
| rAAV | 2080 | WO2016081811A1 SEQ ID NO: 101 |
| rAAV | 2081 | WO2016081811A1 SEQ ID NO: 102 |
| rAAV | 2082 | WO2016081811A1 SEQ ID NO: 103 |
| rAAV | 2083 | WO2016081811A1 SEQ ID NO: 104 |
| rAAV | 2084 | WO2016081811A1 SEQ ID NO: 105 |
| rAAV | 2085 | WO2016081811A1 SEQ ID NO: 106 |
| rAAV | 2086 | WO2016081811A1 SEQ ID NO: 107 |
| rAAV | 2087 | WO2016081811A1 SEQ ID NO: 108 |
| rAAV | 2088 | WO2016081811A1 SEQ ID NO: 109 |
| rAAV | 2089 | WO2016081811A1 SEQ ID NO: 110 |
| rAAV | 2090 | WO2016081811A1 SEQ ID NO: 111 |
| rAAV | 2091 | WO2016081811A1 SEQ ID NO: 112 |
| rAAV | 2092 | WO2016081811A1 SEQ ID NO: 113 |
| rAAV | 2093 | WO2016081811A1 SEQ ID NO: 114 |
| rAAV | 2094 | WO2016081811A1 SEQ ID NO: 115 |
| rAAV | 2095 | WO2016081811A1 SEQ ID NO: 116 |
| rAAV | 2096 | WO2016081811A1 SEQ ID NO: 117 |
| rAAV | 2097 | WO2016081811A1 SEQ ID NO: 118 |
| rAAV | 2098 | WO2016081811A1 SEQ ID NO: 119 |
| rAAV | 2099 | WO2016081811A1 SEQ ID NO: 120 |
| rAAV | 2100 | WO2016081811A1 SEQ ID NO: 121 |
| rAAV | 2101 | WO2016081811A1 SEQ ID NO: 122 |
| rAAV | 2102 | WO2016081811A1 SEQ ID NO: 123 |
| rAAV | 2103 | WO2016081811A1 SEQ ID NO: 124 |
| rAAV | 2104 | WO2016081811A1 SEQ ID NO: 125 |
| rAAV | 2105 | WO2016081811A1 SEQ ID NO: 126 |
| rAAV | 2106 | WO2016081811A1 SEQ ID NO: 127 |
| rAAV | 2107 | WO2016081811A1 SEQ ID NO: 128 |
| AAV8 | 2108 | WO2016081811A1 SEQ ID NO: 133 |
| E532K | | |
| AAV8 | 2109 | WO2016081811A1 SEQ ID NO: 134 |
| E532K | | |
| rAAV4 | 2110 | WO2016115382A1 SEQ ID NO: 2 |
| rAAV4 | 2111 | WO2016115382A1 SEQ ID NO: 3 |
| rAAV4 | 2112 | WO2016115382A1 SEQ ID NO: 4 |
| rAAV4 | 2113 | WO2016115382A1 SEQ ID NO: 5 |
| rAAV4 | 2114 | WO2016115382A1 SEQ ID NO: 6 |
| rAAV4 | 2115 | WO2016115382A1 SEQ ID NO: 7 |
| rAAV4 | 2116 | WO2016115382A1 SEQ ID NO: 8 |
| rAAV4 | 2117 | WO2016115382A1 SEQ ID NO: 9 |
| rAAV4 | 2118 | WO2016115382A1 SEQ ID NO: 10 |
| rAAV4 | 2119 | WO2016115382A1 SEQ ID NO: 11 |
| rAAV4 | 2120 | WO2016115382A1 SEQ ID NO: 12 |
| rAAV4 | 2121 | WO2016115382A1 SEQ ID NO: 13 |
| rAAV4 | 2122 | WO2016115382A1 SEQ ID NO: 14 |
| rAAV4 | 2123 | WO2016115382A1 SEQ ID NO: 15 |
| rAAV4 | 2124 | WO2016115382A1 SEQ ID NO: 16 |
| rAAV4 | 2125 | WO2016115382A1 SEQ ID NO: 17 |
| rAAV4 | 2126 | WO2016115382A1 SEQ ID NO: 18 |
| rAAV4 | 2127 | WO2016115382A1 SEQ ID NO: 19 |
| rAAV4 | 2128 | WO2016115382A1 SEQ ID NO: 20 |
| rAAV4 | 2129 | WO2016115382A1 SEQ ID NO: 21 |
| AAV11 | 2130 | WO2016115382A1 SEQ ID NO: 22 |
| AAV12 | 2131 | WO2016115382A1 SEQ ID NO: 23 |
| rh32 | 2132 | WO2016115382A1 SEQ ID NO: 25 |
| rh33 | 2133 | WO2016115382A1 SEQ ID NO: 26 |
| rh34 | 2134 | WO2016115382A1 SEQ ID NO: 27 |
| rAAV4 | 2135 | WO2016115382A1 SEQ ID NO: 28 |
| rAAV4 | 2136 | WO2016115382A1 SEQ ID NO: 29 |
| rAAV4 | 2137 | WO2016115382A1 SEQ ID NO: 30 |
| rAAV4 | 2138 | WO2016115382A1 SEQ ID NO: 31 |
| rAAV4 | 2139 | WO2016115382A1 SEQ ID NO: 32 |
| rAAV4 | 2140 | WO2016115382A1 SEQ ID NO: 33 |
| AAV2/8 | 2141 | WO2016131981A1 SEQ ID NO: 47 |
| AAV2/8 | 2142 | WO2016131981A1 SEQ ID NO: 48 |
| ancestral | 2143 | WO2016154344A1 SEQ ID NO: 7 |
| AAV | | |
| ancestral | 2144 | WO2016154344A1 SEQ ID NO: 13 |
| AAV variant | | |
| C4 | | |
| ancestral | 2145 | WO2016154344A1 SEQ ID NO: 14 |
| AAV variant | | |
| C7 | | |
| ancestral | 2146 | WO2016154344A1 SEQ ID NO: 15 |
| AAV variant | | |
| G4 | | |
| consensus | 2147 | WO2016154344A1 SEQ ID NO: 16 |
| amino acid | | |
| sequence of | | |
| ancestral | | |
| AAV | | |
| variants, C4, | | |
| C7 and G4 | | |
| consensus | 2148 | WO2016154344A1 SEQ ID NO: 17 |
| amino acid | | |
| sequence of | | |
| ancestral | | |
| AAV | | |
| variants, C4 | | |
| and C7 | | |
| AAV8 (with | 2149 | WO2016150403A1 SEQ ID NO: 13 |
| a AAV2 | | |
| phospholipase | | |
| domain) | | |
| AAV VR- | 2150 | US20160289275A1 SEQ ID NO: 10 |
| 942n | | |
| AAV5-A | 2151 | US20160289275A1 SEQ ID NO: 13 |
| (M569V) | | |
| AAV5-A | 2152 | US20160289275A1 SEQ ID NO: 14 |
| (M569V) | | |
| AAV5-A | 2153 | US20160289275A1 SEQ ID NO: 16 |
| (Y585V) | | |
| AAV5-A | 2154 | US20160289275A1 SEQ ID NO: 17 |
| (Y585V) | | |
| AAV5-A | 2155 | US20160289275A1 SEQ ID NO: 19 |
| (L587T) | | |
| AAV5-A | 2156 | US20160289275A1 SEQ ID NO: 20 |
| (L587T) | | |
| AAV5-A | 2157 | US20160289275A1 SEQ ID NO: 22 |
| (Y585V/L587T) | | |
| AAV5-A | 2158 | US20160289275A1 SEQ ID NO: 23 |
| (Y585V/L587T) | | |
| AAV5-B | 2159 | US20160289275A1 SEQ ID NO: 25 |
| (D652A) | | |
| AAV5-B | 2160 | US20160289275A1 SEQ ID NO: 26 |
| (D652A) | | |
| AAV5-B | 2161 | US20160289275A1 SEQ ID NO: 28 |
| (T362M) | | |
| AAV5-B | 2162 | US20160289275A1 SEQ ID NO: 29 |
| (T362M) | | |
| AAV5-B | 2163 | US20160289275A1 SEQ ID NO: 31 |
| (Q359D) | | |
| AAV5-B | 2164 | US20160289275A1 SEQ ID NO: 32 |
| (Q359D) | | |
| AAV5-B | 2165 | US20160289275A1 SEQ ID NO: 34 |
| (E350Q) | | |
| AAV5-B | 2166 | US20160289275A1 SEQ ID NO: 35 |
| (E350Q) | | |
| AAV5-B | 2167 | US20160289275A1 SEQ ID NO: 37 |
| (P533S) | | |
| AAV5-B | 2168 | US20160289275A1 SEQ ID NO: 38 |
| (P533S) | | |
| AAV5-B | 2169 | US20160289275A1 SEQ ID NO: 40 |
| (P533G) | | |
| AAV5-B | 2170 | US20160289275A1 SEQ ID NO: 41 |
| (P533G) | | |
| AAV5- | 2171 | US20160289275A1 SEQ ID NO: 43 |
| mutation in | | |
| loop VII | | |
| AAV5- | 2172 | US20160289275A1 SEQ ID NO: 44 |
| mutation in | | |
| loop VII | | |
| AAV8 | 2173 | US20160289275A1 SEQ ID NO: 47 |
| Mut A | 2174 | WO2016181123A1 SEQ ID NO: 1 |
| (LK03/AAV8) | | |
| Mut B | 2175 | WO2016181123A1 SEQ ID NO: 2 |
| (LK03/AAV5) | | |
| Mut C | 2176 | WO2016181123A1 SEQ ID NO: 3 |
| (AAV8/ | | |
| AAV3B) | | |
| Mut D | 2177 | WO2016181123A1 SEQ ID NO: 4 |
| (AAV5/ | | |
| AAV3B) | | |
| Mut E | 2178 | WO2016181123A1 SEQ ID NO: 5 |
| (AAV8/ | | |
| AAV3B) | | |
| Mut F | 2179 | WO2016181123A1 SEQ ID NO: 6 |
| (AAV3B/ | | |
| AAV8) | | |
| AAV44.9 | 2180 | WO2016183297A1 SEQ ID NO: 4 |
| AAV44.9 | 2181 | WO2016183297A1 SEQ ID NO: 5 |
| AAVrh8 | 2182 | WO2016183297A1 SEQ ID NO: 6 |
| AAV44.9 | 2183 | WO2016183297A1 SEQ ID NO: 9 |
| (S470N) | | |
| rh74 VP1 | 2184 | US20160375110A1 SEQ ID NO: 1 |
| AAV-LK03 | 2185 | WO2017015102A1 SEQ ID NO: 5 |
| (L135I) | | |
| AAV3B | 2186 | WO2017015102A1 SEQ ID NO: 6 |
| (S663V + | | |
| T492V) | | |
| Anc80 | 2187 | WO2017019994A2 SEQ ID NO: 1 |
| Anc80 | 2188 | WO2017019994A2 SEQ ID NO: 2 |
| Anc81 | 2189 | WO2017019994A2 SEQ ID NO: 3 |
| Anc81 | 2190 | WO2017019994A2 SEQ ID NO: 4 |
| Anc82 | 2191 | WO2017019994A2 SEQ ID NO: 5 |
| Anc82 | 2192 | WO2017019994A2 SEQ ID NO: 6 |
| Anc83 | 2193 | WO2017019994A2 SEQ ID NO: 7 |
| Anc83 | 2194 | WO2017019994A2 SEQ ID NO: 8 |
| Anc84 | 2195 | WO2017019994A2 SEQ ID NO: 9 |
| Anc84 | 2196 | WO2017019994A2 SEQ ID NO: 10 |
| Anc94 | 2197 | WO2017019994A2 SEQ ID NO: 11 |
| Anc94 | 2198 | WO2017019994A2 SEQ ID NO: 12 |
| Anc113 | 2199 | WO2017019994A2 SEQ ID NO: 13 |
| Anc113 | 2200 | WO2017019994A2 SEQ ID NO: 14 |
| Anc126 | 2201 | WO2017019994A2 SEQ ID NO: 15 |
| Anc126 | 2202 | WO2017019994A2 SEQ ID NO: 16 |
| Anc127 | 2203 | WO2017019994A2 SEQ ID NO: 17 |
| Anc127 | 2204 | WO2017019994A2 SEQ ID NO: 18 |
| Anc80L27 | 2205 | WO2017019994A2 SEQ ID NO: 19 |
| Anc80L59 | 2206 | WO2017019994A2 SEQ ID NO: 20 |
| Anc80L60 | 2207 | WO2017019994A2 SEQ ID NO: 21 |
| Anc80L62 | 2208 | WO2017019994A2 SEQ ID NO: 22 |
| Anc80L65 | 2209 | WO2017019994A2 SEQ ID NO: 23 |
| Anc80L33 | 2210 | WO2017019994A2 SEQ ID NO: 24 |
| Anc80L36 | 2211 | WO2017019994A2 SEQ ID NO: 25 |
| Anc80L44 | 2212 | WO2017019994A2 SEQ ID NO: 26 |
| Anc80L1 | 2213 | WO2017019994A2 SEQ ID NO: 35 |
| Anc80L1 | 2214 | WO2017019994A2 SEQ ID NO: 36 |
| AAVrh10 | 2215 | WO2017019994A2 SEQ ID NO: 41 |
| Anc110 | 2216 | WO2017019994A2 SEQ ID NO: 42 |
| Anc110 | 2217 | WO2017019994A2 SEQ ID NO: 43 |
| AAVrh32.33 | 2218 | WO2017019994A2 SEQ ID NO: 45 |
| AAVrh74 | 2219 | WO2017049031A1 SEQ ID NO: 1 |
| AAV2 | 2220 | WO2017053629A2 SEQ ID NO: 49 |
| AAV2 | 2221 | WO2017053629A2 SEQ ID NO: 50 |
| AAV2 | 2222 | WO2017053629A2 SEQ ID NO: 82 |
| Parvo-like | 2223 | WO2017070476A2 SEQ ID NO: 1 |
| virus | | |
| Parvo-like | 2224 | WO2017070476A2 SEQ ID NO: 2 |
| virus | | |
| Parvo-like | 2225 | WO2017070476A2 SEQ ID NO: 3 |
| virus | | |
| Parvo-like | 2226 | WO2017070476A2 SEQ ID NO: 4 |
| virus | | |
| Parvo-like | 2227 | WO2017070476A2 SEQ ID NO: 5 |
| virus | | |
| Parvo-like | 2228 | WO2017070476A2 SEQ ID NO: 6 |
| virus | | |
| AAVrh.10 | 2229 | WO2017070516A1 SEQ ID NO: 7 |
| AAVrh.10 | 2230 | WO2017070516A1 SEQ ID NO: 14 |
| AAV2tYF | 2231 | WO2017070491A1 SEQ ID NO: 1 |
| AAV-SPK | 2232 | WO2017075619A1 SEQ ID NO: 28 |
| AAV2.5 | 2233 | US20170128528A1 SEQ ID NO: 13 |
| AAV1.1 | 2234 | US20170128528A1 SEQ ID NO: 15 |
| AAV6.1 | 2235 | US20170128528A1 SEQ ID NO: 17 |
| AAV6.3.1 | 2236 | US20170128528A1 SEQ ID NO: 18 |
| AAV2i8 | 2237 | US20170128528A1 SEQ ID NO: 28 |
| AAV2i8 | 2238 | US20170128528A1 SEQ ID NO: 29 |
| ttAAV | 2239 | US20170128528A1 SEQ ID NO: 30 |
| ttAAV- | 2240 | US20170128528A1 SEQ ID NO: 32 |
| S312N | | |
| ttAAV- | 2241 | US20170128528A1 SEQ ID NO: 33 |
| S312N | | |
| AAV6 | 2242 | WO2016134337A1 SEQ ID NO: 24 |
| (Y705, Y731, | | |
| and T492) | | |
| AAV2 | 2243 | WO2016134375A1 SEQ ID NO: 9 |
| AAV2 | 2244 | WO2016134375A1 SEQ ID NO: 10 |

Each of the patents, applications and/or publications listed in Table 1 are hereby incorporated by reference in their entirety.

In one embodiment, the AAV serotype may be, or may have a sequence as described in International Patent Publication WO2015038958, the contents of which are herein incorporated by reference in their entirety, such as, but not limited to, AAV9 (SEQ ID NO: 2 and 11 of WO2015038958 or SEQ ID NO: 127 and 126 respectively herein), PHP.B (SEQ ID NO: 8 and 9 of WO2015038958, herein SEQ ID NO: 868 and 869), G2B-13 (SEQ ID NO: 12 of WO2015038958, herein SEQ ID NO: 870), G2B-26 (SEQ ID NO: 13 of WO2015038958, herein SEQ ID NO: 868 and 869), TH1.1-32 (SEQ ID NO: 14 of WO2015038958, herein SEQ ID NO: 871), TH1.1-35 (SEQ ID NO: 15 of WO2015038958, herein SEQ ID NO: 872) or variants thereof. Further, any of the targeting peptides or amino acid inserts described in WO2015038958, may be inserted into any parent AAV serotype, such as, but not limited to, AAV9 (SEQ ID NO: 126 for the DNA sequence and SEQ ID NO: 127 for the amino acid sequence). In one embodiment, the amino acid insert is inserted between amino acids 586-592 of the parent AAV (e.g., AAV9). In another embodiment, the amino acid insert is inserted between amino acids 588-589 of the parent AAV sequence. The amino acid insert may be, but is not limited to, any of the following amino acid sequences, TLAVPFK (SEQ ID NO: 1 of WO2015038958; herein SEQ ID NO: 873), KFPVALT (SEQ ID NO: 3 of WO2015038958; herein SEQ ID NO: 874), LAVPFK (SEQ ID NO: 31 of WO2015038958; herein SEQ ID NO: 875), AVPFK (SEQ ID NO: 32 of WO2015038958; herein SEQ ID NO: 876), VPFK (SEQ ID NO: 33 of WO2015038958; herein SEQ ID NO: 877), TLAVPF (SEQ ID NO: 34 of WO2015038958; herein SEQ ID NO: 878), TLAVP (SEQ ID NO: 35 of WO2015038958; herein SEQ ID NO: 879), TLAV (SEQ ID NO: 36 of WO2015038958; herein SEQ ID NO: 880), SVSKPFL (SEQ ID NO: 28 of WO2015038958; herein SEQ ID NO: 881), FTLTTPK (SEQ ID NO: 29 of WO2015038958; herein SEQ ID NO: 882), MNATKNV (SEQ ID NO: 30 of WO2015038958; herein SEQ ID NO: 883), QSSQTPR (SEQ ID NO: 54 of WO2015038958; herein SEQ ID NO: 884), ILGTGTS (SEQ ID NO: 55 of WO2015038958; herein SEQ ID NO: 885), TRTNPEA (SEQ ID NO: 56 of WO2015038958; herein SEQ ID NO: 886), NGGTSSS (SEQ ID NO: 58 of WO2015038958; herein SEQ ID NO: 887), or YTLSQGW (SEQ ID NO: 60 of WO2015038958; herein SEQ ID NO: 888). Non-limiting examples of nucleotide sequences that may encode the amino acid inserts include the following, AAGTTTCCTGTGGCGTTGACT (for SEQ ID NO: 3 of WO2015038958; herein SEQ ID NO: 889), ACTTTGGCGGTGCCTTTTAAG (SEQ ID NO: 24 and 49 of WO2015038958; herein SEQ ID NO: 890), AGTGTGAGTAAGCCTTTTTTG (SEQ ID NO: 25 of WO2015038958; herein SEQ ID NO: 891), TTTACGTTGACGACGCCTAAG (SEQ ID NO: 26 of WO2015038958; herein SEQ ID NO: 892), ATGAATGCTACGAAGAATGTG (SEQ ID NO: 27 of WO2015038958; herein SEQ ID NO: 893), CAGTCGTCGCAGACGCCTAGG (SEQ ID NO: 48 of WO2015038958; herein SEQ ID NO: 894), ATTCTGGGGACTGGTACTTCG (SEQ ID NO: 50 and 52 of WO2015038958; herein SEQ ID NO: 895), ACGCGGACTAATCCTGAGGCT (SEQ ID NO: 51 of WO2015038958; herein SEQ ID NO: 896), AATGGGGGGACTAGTAGTTCT (SEQ ID NO: 53 of WO2015038958; herein SEQ ID NO: 897), or TATACTTTGTCGCAGGGTTGG (SEQ ID NO: 59 of WO2015038958; herein SEQ ID NO: 898).

In one embodiment, the AAV serotype may be engineered to comprise at least one AAV capsid CD8+ T-cell epitope for AAV2 such as, but not limited to, SADNNNSEY (SEQ ID NO: 899), LIDQYLYYL (SEQ ID NO: 900), VPQYGYLTL (SEQ ID NO: 901), TTSTRTWAL (SEQ ID NO: 902), YHLNGRDSL (SEQ ID NO: 903), SQAVGRSSF (SEQ ID NO: 904), VPANPSTTF (SEQ ID NO: 905), FPQSGVLIF (SEQ ID NO: 906), YFDFNRFHCHFSPRD (SEQ ID NO: 907), VGNSSGNWHCDSTWM (SEQ ID NO: 908), QFSQAGASDIRDQSR (SEQ ID NO: 909), GASDIRQSRNWLP (SEQ ID NO: 910) and GNRQAATADVNTQGV (SEQ ID NO: 911).

In one embodiment, the AAV serotype may be engineered to comprise at least one AAV capsid CD8+ T-cell epitope for AAV1 such as, but not limited to, LDRLMNPLI (SEQ ID NO: 912), TTSTRTWAL (SEQ ID NO: 902), and QPAKKRLNF (SEQ ID NO: 913)).

In one embodiment, the AAV serotype may be, or may have a sequence as described in International Patent Publication WO2017100671, the contents of which are herein incorporated by reference in their entirety, such as, but not limited to, AAV9 (SEQ ID NO: 45 of WO2017100671, herein SEQ ID NO: 1861), PHP.N (SEQ ID NO: 46 of WO2017100671, herein SEQ ID NO: 1859), PHP.S (SEQ ID NO: 47 of WO2017100671, herein SEQ ID NO: 1860), or variants thereof. Further, any of the targeting peptides or amino acid inserts described in WO2017100671 may be inserted into any parent AAV serotype, such as, but not limited to, AAV9 (SEQ ID NO: 127 or SEQ ID NO: 1861). In one embodiment, the amino acid insert is inserted between amino acids 586-592 of the parent AAV (e.g., AAV9). In another embodiment, the amino acid insert is inserted between amino acids 588-589 of the parent AAV sequence. The amino acid insert may be, but is not limited to, any of the following amino acid sequences, AQTLAVPFKAQ (SEQ ID NO: 1 of WO2017100671; herein SEQ ID NO: 2245), AQSVSKPFLAQ (SEQ ID NO: 2 of WO2017100671; herein SEQ ID NO: 2246), AQFTLTTPKAQ (SEQ ID NO: 3 in the sequence listing of WO2017100671; herein SEQ ID NO: 2247), DGTLAVPFKAQ (SEQ ID NO: 4 in the sequence listing of WO2017100671; herein SEQ ID NO: 2248), ESTLAVPFKAQ (SEQ ID NO: 5 of WO2017100671; herein SEQ ID NO: 2249), GGTLAVPFKAQ (SEQ ID NO: 6 of WO2017100671; herein SEQ ID NO: 2250), AQTLATPFKAQ (SEQ ID NO: 7 and 33 of WO2017100671; herein SEQ ID NO: 2251), ATTLATPFKAQ (SEQ ID NO: 8 of WO2017100671; herein SEQ ID NO: 2252), DGTLATPFKAQ (SEQ ID NO: 9 of WO2017100671; herein SEQ ID NO: 2253), GGTLATPFKAQ (SEQ ID NO: 10 of WO2017100671; herein SEQ ID NO: 2254), SGSLAVPFKAQ (SEQ ID NO: 11 of WO2017100671; herein SEQ ID NO: 2255), AQTLAQPFKAQ (SEQ ID NO: 12 of WO2017100671; herein SEQ ID NO: 2256), AQTLQQPFKAQ (SEQ ID NO: 13 of WO2017100671; herein SEQ ID NO: 2257), AQTLSNPFKAQ (SEQ ID NO: 14 of WO2017100671; herein SEQ ID NO: 2258), AQTLAVPFSNP (SEQ ID NO: 15 of WO2017100671; herein SEQ ID NO: 2259), QGTLAVPFKAQ (SEQ ID NO: 16 of WO2017100671; herein SEQ ID NO: 2260), NQTLAVPFKAQ (SEQ ID NO: 17 of WO2017100671; herein SEQ ID NO: 2261), EGSLAVPFKAQ (SEQ ID NO: 18 of WO2017100671; herein SEQ ID NO: 2262), SGNLAVPFKAQ (SEQ ID NO: 19 of WO2017100671; herein SEQ ID NO: 2263), EGTLAVPFKAQ (SEQ ID NO: 20 of WO2017100671; herein SEQ ID NO: 2264), DSTLAVPFKAQ (SEQ ID NO: 21 in Table 1 of WO2017100671; herein SEQ ID NO: 2265), AVTLAVPFKAQ (SEQ ID NO: 22 of WO2017100671; herein SEQ ID NO: 2266), AQTLSTPFKAQ (SEQ ID NO: 23 of WO2017100671; herein SEQ ID NO: 2267), AQTLPQPFKAQ (SEQ ID NO: 24 and 32 of WO2017100671; herein SEQ ID NO: 2268), AQTLSQPFKAQ (SEQ ID NO: 25 of WO2017100671; herein SEQ ID NO: 2269), AQTLQLPFKAQ (SEQ ID NO: 26 of WO2017100671; herein SEQ ID NO: 2270), AQTLTMPFKAQ (SEQ ID NO: 27, and 34 of WO2017100671 and SEQ ID NO: 35 in the sequence listing of WO2017100671; herein SEQ ID NO: 2271), AQTLTTPFKAQ (SEQ ID NO: 28 of WO2017100671; herein SEQ ID NO: 2272), AQYTLSQGWAQ (SEQ ID NO: 29 of WO2017100671; herein SEQ ID NO: 2273), AQMNATKNVAQ (SEQ ID NO: 30 of WO2017100671; herein SEQ ID NO: 2274), AQVSGGHHSAQ (SEQ ID NO: 31 of WO2017100671; herein SEQ ID NO: 2275), AQTLTAPFKAQ (SEQ ID NO: 35 in Table 1 of WO2017100671; herein SEQ ID NO: 2276), AQTLSKPFKAQ (SEQ ID NO: 36 of WO2017100671; herein SEQ ID NO: 2277), QAVRTSL (SEQ ID NO: 37 of WO2017100671; herein SEQ ID NO: 2278), YTLSQGW (SEQ ID NO: 38 of WO2017100671; herein SEQ ID NO: 888), LAKERLS (SEQ ID NO: 39 of WO2017100671; herein SEQ ID NO: 2279), TLAVPFK (SEQ ID NO: 40 in the sequence listing of WO2017100671; herein SEQ ID NO: 873), SVSKPFL (SEQ ID NO: 41 of WO2017100671; herein SEQ ID NO: 881), FTLTTPK (SEQ ID NO: 42 of WO2017100671; herein SEQ ID NO: 882), MNSTKNV (SEQ ID NO: 43 of WO2017100671; herein SEQ ID NO: 2280), VSGGHHS (SEQ ID NO: 44 of WO2017100671; herein SEQ ID NO: 2281), SAQTLAVPFKAQAQ (SEQ ID NO: 48 of WO2017100671; herein SEQ ID NO: 2282), SXXXLAVPFKAQAQ (SEQ ID NO: 49 of WO2017100671 wherein X may be any amino acid; herein SEQ ID NO: 2283), SAQXXXVPFKAQAQ (SEQ ID NO: 50 of WO2017100671 wherein X may be any amino acid; herein SEQ ID NO: 2284), SAQTLXXXFKAQAQ (SEQ ID NO: 51 of WO2017100671 wherein X may be any amino acid; herein SEQ ID NO: 2285), SAQTLAVXXXAQAQ (SEQ ID NO: 52 of WO2017100671 wherein X may be any amino acid; herein SEQ ID NO: 2286), SAQTLAVPFXXXAQ (SEQ ID NO: 53 of WO2017100671 wherein X may be any amino acid; herein SEQ ID NO: 2287), TNHQSAQ (SEQ ID NO: 65 of WO2017100671; herein SEQ ID NO: 2288), AQAQTGW (SEQ ID NO: 66 of WO2017100671; herein SEQ ID NO: 2289), DGTLATPFK (SEQ ID NO: 67 of WO2017100671; herein SEQ ID NO: 2290), DGTLATPFKXX (SEQ ID NO: 68 of WO2017100671 wherein X may be any amino acid; herein SEQ ID NO: 2291), LAVPFKAQ (SEQ ID NO: 80 of WO2017100671; herein SEQ ID NO: 2292), VPFKAQ (SEQ ID NO: 81 of WO2017100671; herein SEQ ID NO: 2293), FKAQ (SEQ ID NO: 82 of WO2017100671; herein SEQ ID NO: 2294), AQTLAV (SEQ ID NO: 83 of WO2017100671; herein SEQ ID NO: 2295), AQTLAVPF (SEQ ID NO: 84 of WO2017100671; herein SEQ ID NO: 2296), QAVR (SEQ ID NO: 85 of WO2017100671; herein SEQ ID NO: 2297), AVRT (SEQ ID NO: 86 of WO2017100671; herein SEQ ID NO: 2298), VRTS (SEQ ID NO: 87 of WO2017100671; herein SEQ ID NO: 2299), RTSL (SEQ ID NO: 88 of WO2017100671; herein SEQ ID NO: 2300), QAVRT (SEQ ID NO: 89 of WO2017100671; herein SEQ ID NO: 2301), AVRTS (SEQ ID NO: 90 of WO2017100671; herein SEQ ID NO: 2302), VRTSL (SEQ ID NO: 91 of WO2017100671; herein SEQ ID NO: 2303), QAVRTS (SEQ ID NO: 92 of WO2017100671; herein SEQ ID NO: 2304), or AVRTSL (SEQ ID NO: 93 of WO2017100671; herein SEQ ID NO: 2305).

Non-limiting examples of nucleotide sequences that may encode the amino acid inserts include the following, GATGGGACTTTGGCGGTGCCTTTTAAGGCACAG (SEQ ID NO: 54 of WO2017100671; herein SEQ ID NO: 2306), GATGGGACGTTGGCGGTGCCTTTTAAGGCACAG (SEQ ID NO: 55 of WO2017100671; herein SEQ ID NO: 2307), CAGGCGGTTAGGACGTCTTTG (SEQ ID NO: 56 of WO2017100671; herein SEQ ID NO: 2308), CAGGTCTTCACGGACTCAGACTATCAG (SEQ ID NO: 57 and 78 of WO2017100671; herein SEQ ID NO: 2309), CAAGTAAAACCTCTACAAATGTGGTAAAATCG (SEQ ID NO: 58 of WO2017100671; herein SEQ ID NO: 2310), ACTCATCGACCAATACTTGTACTATCTCTCTAGAAC (SEQ ID NO: 59 of WO2017100671; herein SEQ ID NO: 2311), GGAAGTATTCCTTGGTTTTGAACCCA (SEQ ID NO: 60 of WO2017100671; herein SEQ ID NO: 2312), GGTCGCGGTTCTTGTTTGTGGAT (SEQ ID NO: 61 of WO2017100671; herein SEQ ID NO: 2313), CGACCTTGAAGCGCATGAACTCCT (SEQ ID NO: 62 of WO2017100671; herein SEQ ID NO: 2314), GTATTCCTTGGTTTTGAACCCAACCGGTCTGCGCCTGTGCMNNMNNMNNMNNMNN MNNMNNTTGGGCACTCTGGTGGTTTGTC (SEQ ID NO: 63 of WO2017100671 wherein N may be A, C, T, or G; herein SEQ ID NO: 2315), GTATTCCTTGGTTTTGAACCCAACCGGTCTGCGCMNNMNNMNNAAAAGGCACCGCC AAAGTTTG (SEQ ID NO: 69 of WO2017100671 wherein N may be A, C, T, or G; herein SEQ ID NO: 2316), GTATTCCTTGGTTTTGAACCCAACCGGTCTGCGCCTGTGCMNNMNNMNNCACCGCC AAAGTTTGGGCACT (SEQ ID NO: 70 of WO2017100671 wherein N may be A, C, T, or G; herein SEQ ID NO: 2317), GTATTCCTTGGTTTTGAACCCAACCGGTCTGCGCCTGTGCCTTAAAMNNMNNMNNC AAAGTTTGGGCACTCTGGTGG (SEQ ID NO: 71 of WO2017100671 wherein N may be A, C, T, or G; herein SEQ ID NO: 2318), GTATTCCTTGGTTTTGAACCCAACCGGTCTGCGCCTGTGCCTTAAAAGGCACMNNM NNMNNTTGGGCACTCTGGTGGTTTGTG (SEQ ID NO: 72 of WO2017100671 wherein N may be A, C, T, or G; herein SEQ ID NO: 2319), ACTTTGGCGGTGCCTTTTAAG (SEQ ID NO: 74 of WO2017100671; herein SEQ ID NO: 890), AGTGTGAGTAAGCCTTTTTTG (SEQ ID NO: 75 of WO2017100671; herein SEQ ID NO: 891), TTTACGTTGACGACGCCTAAG (SEQ ID NO: 76 of WO2017100671; herein SEQ ID NO: 892), TATACTTTGTCGCAGGGTTGG (SEQ ID NO: 77 of WO2017100671; herein SEQ ID NO: 898), or CTTGCGAAGGAGCGGCTTTCG (SEQ ID NO: 79 of WO2017100671; herein SEQ ID NO: 2320).

In one embodiment, the AAV serotype may be, or may have a sequence as described in U.S. Pat. No. 9,624,274, the contents of which are herein incorporated by reference in their entirety, such as, but not limited to, AAV1 (SEQ ID NO: 181 of U.S. Pat. No. 9,624,274), AAV6 (SEQ ID NO: 182 of U.S. Pat. No. 9,624,274), AAV2 (SEQ ID NO: 183 of U.S. Pat. No. 9,624,274), AAV3b (SEQ ID NO: 184 of U.S. Pat. No. 9,624,274), AAV7 (SEQ ID NO: 185 of U.S. Pat. No. 9,624,274), AAV8 (SEQ ID NO: 186 of U.S. Pat. No. 9,624,274), AAV10 (SEQ ID NO: 187 of U.S. Pat. No. 9,624,274), AAV4 (SEQ ID NO: 188 of U.S. Pat. No. 9,624,274), AAV11 (SEQ ID NO: 189 of U.S. Pat. No. 9,624,274), bAAV (SEQ ID NO: 190 of U.S. Pat. No. 9,624,274), AAV5 (SEQ ID NO: 191 of U.S. Pat. No. 9,624,274), GPV (SEQ ID NO: 192 of U.S. Pat. No. 9,624,274; herein SEQ ID NO: 1862), B19 (SEQ ID NO: 193 of U.S. Pat. No. 9,624,274; herein SEQ ID NO: 1863), MVM (SEQ ID NO: 194 of U.S. Pat. No. 9,624,274; herein SEQ ID NO: 1864), FPV (SEQ ID NO: 195 of U.S. Pat. No. 9,624,274; herein SEQ ID NO: 1865), CPV (SEQ ID NO: 196 of U.S. Pat. No. 9,624,274; herein SEQ ID NO: 1866) or variants thereof. Further, any of the structural protein inserts described in U.S. Pat. No. 9,624,274, may be inserted into, but not limited to, 1-453 and 1-587 of any parent AAV serotype, such as, but not limited to, AAV2 (SEQ ID NO: 183 of U.S. Pat. No. 9,624,274). The amino acid insert may be, but is not limited to, any of the following amino acid sequences, VNLTWSRASG (SEQ ID NO: 50 of U.S. Pat. No. 9,624,274; herein SEQ ID NO: 2321), EFCINHRGYWVCGD (SEQ ID NO:55 of U.S. Pat. No. 9,624,274; herein SEQ ID NO: 2322), EDGQVMDVDLS (SEQ ID NO: 85 of U.S. Pat. No. 9,624,274; herein SEQ ID NO: 2323), EKQRNGTLT (SEQ ID NO: 86 of U.S. Pat. No. 9,624,274; herein SEQ ID NO: 2324), TYQCRVTHPHLPRALMR (SEQ ID NO: 87 of U.S. Pat. No. 9,624,274; herein SEQ ID NO: 2325), RHSTTQPRKTKGSG (SEQ ID NO: 88 of U.S. Pat. No. 9,624,274; herein SEQ ID NO: 2326), DSNPRGVSAYLSR (SEQ ID NO: 89 of U.S. Pat. No. 9,624,274; herein SEQ ID NO: 2327), TITCLWDLAPSK (SEQ ID NO: 90 of U.S. Pat. No. 9,624,274; herein SEQ ID NO: 2328), KTKGSGFFVF (SEQ ID NO: 91 of U.S. Pat. No. 9,624,274; herein SEQ ID NO: 2329), THPHLPRALMRS (SEQ ID NO: 92 of U.S. Pat. No. 9,624,274; herein SEQ ID NO: 2330), GETYQCRVTHPHLPRALMRSTTK (SEQ ID NO: 93 of U.S. Pat. No. 9,624,274; herein SEQ ID NO: 2331), LPRALMRS (SEQ ID NO: 94 of U.S. Pat. No. 9,624,274; herein SEQ ID NO: 2332), INHRGYWV (SEQ ID NO: 95 of U.S. Pat. No. 9,624,274; herein SEQ ID NO: 2333), CDAGSVRTNAPD (SEQ ID NO: 60 of U.S. Pat. No. 9,624,274; herein SEQ ID NO: 2334), AKAVSNLTESRSESLQS (SEQ ID NO: 96 of U.S. Pat. No. 9,624,274; herein SEQ ID NO: 2335), SLTGDEFKKVLET (SEQ ID NO: 97 of U.S. Pat. No. 9,624,274; herein SEQ ID NO: 2336), REAVAYRFEED (SEQ ID NO: 98 of U.S. Pat. No. 9,624,274; herein SEQ ID NO: 2337), INPEIITLDG (SEQ ID NO: 99 of U.S. Pat. No. 9,624,274; herein SEQ ID NO: 2338), DISVTGAPVITATYL (SEQ ID NO: 100 of U.S. Pat. No. 9,624,274; herein SEQ ID NO: 2339), DISVTGAPVITA (SEQ ID NO: 101 of U.S. Pat. No. 9,624,274; herein SEQ ID NO: 2340), PKTVSNLTESSSESVQS (SEQ ID NO: 102 of U.S. Pat. No. 9,624,274; herein SEQ ID NO: 2341), SLMGDEFKAVLET (SEQ ID NO: 103 of U.S. Pat. No. 9,624,274; herein SEQ ID NO: 2342), QHSVAYTFEED (SEQ ID NO: 104 of U.S. Pat. No. 9,624,274; herein SEQ ID NO: 2343), INPEIITRDG (SEQ ID NO: 105 of U.S. Pat. No. 9,624,274; herein SEQ ID NO: 2344), DISLTGDPVITASYL (SEQ ID NO: 106 of U.S. Pat. No. 9,624,274; herein SEQ ID NO: 2345), DISLTGDPVITA (SEQ ID NO: 107 of U.S. Pat. No. 9,624,274; herein SEQ ID NO: 2346), DQSIDFEIDSA (SEQ ID NO: 108 of U.S. Pat. No. 9,624,274; herein SEQ ID NO: 2347), KNVSEDLPLPTFSPTLLGDS (SEQ ID NO: 109 of U.S. Pat. No. 9,624,274; herein SEQ ID NO: 2348), KNVSEDLPLPT (SEQ ID NO: 110 of U.S. Pat. No. 9,624,274; herein SEQ ID NO: 2349), CDSGRVRTDAPD (SEQ ID NO: 111 of U.S. Pat. No. 9,624,274; herein SEQ ID NO: 2350), FPEHLLVDFLQSLS (SEQ ID NO: 112 of U.S. Pat. No. 9,624,274; herein SEQ ID NO: 2351), DAEFRHDSG (SEQ ID NO: 65 of U.S. Pat. No. 9,624,274; herein SEQ ID NO: 2352), HYAAAQWDFGNTMCQL (SEQ ID NO: 113 of U.S. Pat. No. 9,624,274; herein SEQ ID NO: 2353), YAAQWDFGNTMCQ (SEQ ID NO: 114 of U.S. Pat. No. 9,624,274; herein SEQ ID NO: 2354), RSQKEGLHYT (SEQ ID NO: 115 of U.S. Pat. No. 9,624,274; herein SEQ ID NO: 2355), SSRTPSDKPVAHWANPQAE (SEQ ID NO: 116 of U.S. Pat. No. 9,624,274; herein SEQ ID NO: 2356), SRTPSDKPVAHWANP (SEQ ID NO: 117 of U.S. Pat. No. 9,624,274; herein SEQ ID NO: 2357), SSRTPSDKP (SEQ ID NO: 118 of U.S. Pat. No. 9,624,274; herein SEQ ID NO: 2358), NADGNVDYHMNSVP (SEQ ID NO: 119 of U.S. Pat. No. 9,624,274; herein SEQ ID NO: 2359), DGNVDYHMNSV (SEQ ID NO: 120 of U.S. Pat. No. 9,624,274; herein SEQ ID NO: 2360), RSFKEFLQSSLRALRQ (SEQ ID NO: 121 of U.S. Pat. No. 9,624,274; herein SEQ ID NO: 2361); FKEFLQSSLRA (SEQ ID NO: 122 of U.S. Pat. No. 9,624,274; herein SEQ ID NO: 2362), or QMWAPQWGPD (SEQ ID NO: 123 of U.S. Pat. No. 9,624,274; herein SEQ ID NO: 2363).

In one embodiment, the AAV serotype may be, or may have a sequence as described in U.S. Pat. No. 9,475,845, the contents of which are herein incorporated by reference in their entirety, such as, but not limited to, AAV capsid proteins comprising modification of one or more amino acids at amino acid positions 585 to 590 of the native AAV2 capsid protein. Further the modification may result in, but not limited to, the amino acid sequence RGNRQA (SEQ ID NO: 3 of U.S. Pat. No. 9,475,845; herein SEQ ID NO: 2364), SSSTDP (SEQ ID NO: 4 of U.S. Pat. No. 9,475,845; herein SEQ ID NO: 2365), SSNTAP (SEQ ID NO: 5 of U.S. Pat. No. 9,475,845; herein SEQ ID NO: 2366), SNSNLP (SEQ ID NO: 6 of U.S. Pat. No. 9,475,845; herein SEQ ID NO: 2367), SSTTAP (SEQ ID NO: 7 of U.S. Pat. No. 9,475,845; herein SEQ ID NO: 2368), AANTAA (SEQ ID NO: 8 of U.S. Pat. No. 9,475,845; herein SEQ ID NO: 2369), QQNTAP (SEQ ID NO: 9 of U.S. Pat. No. 9,475,845; herein SEQ ID NO: 2370), SAQAQA (SEQ ID NO: 10 of U.S. Pat. No. 9,475,845; herein SEQ ID NO: 2371), QANTGP (SEQ ID NO: 11 of U.S. Pat. No. 9,475,845; herein SEQ ID NO: 2372), NATTAP (SEQ ID NO: 12 of U.S. Pat. No. 9,475,845; herein SEQ ID NO: 2373), SSTAGP (SEQ ID NO: 13 and 20 of U.S. Pat. No. 9,475,845; herein SEQ ID NO: 2374), QQNTAA (SEQ ID NO: 14 of U.S. Pat. No. 9,475,845; herein SEQ ID NO: 2375), PSTAGP (SEQ ID NO: 15 of U.S. Pat. No. 9,475,845; herein SEQ ID NO: 2376), NQNTAP (SEQ ID NO: 16 of U.S. Pat. No. 9,475,845; herein SEQ ID NO: 2377), QAANAP (SEQ ID NO: 17 of U.S. Pat. No. 9,475,845; herein SEQ ID NO: 2378), SIVGLP (SEQ ID NO: 18 of U.S. Pat. No. 9,475,845; herein SEQ ID NO: 2379), AASTAA (SEQ ID NO: 19, and 27 of U.S. Pat. No. 9,475,845; herein SEQ ID NO: 2380), SQNTTA (SEQ ID NO: 21 of U.S. Pat. No. 9,475,845; herein SEQ ID NO: 2381), QQDTAP (SEQ ID NO: 22 of U.S. Pat. No. 9,475,845; herein SEQ ID NO: 2382), QTNTGP (SEQ ID NO: 23 of U.S. Pat. No. 9,475,845; herein SEQ ID NO: 2383), QTNGAP (SEQ ID NO: 24 of U.S. Pat. No. 9,475,845; herein SEQ ID NO: 2384), QQNAAP (SEQ ID NO: 25 of U.S. Pat. No. 9,475,845; herein SEQ ID NO: 2385), or AANTQA (SEQ ID NO: 26 of U.S. Pat. No. 9,475,845; herein SEQ ID NO: 2386). In one embodiment, the amino acid modification is a substitution at amino acid positions 262 through 265 in the native AAV2 capsid protein or the corresponding position in the capsid protein of another AAV with a targeting sequence. The targeting sequence may be, but is not limited to, any of the amino acid sequences, NGRAHA (SEQ ID NO: 38 of U.S. Pat. No. 9,475,845; herein SEQ ID NO: 2387), QPEHSST (SEQ ID NO: 39 and 50 of U.S. Pat. No. 9,475,845; herein SEQ ID NO: 2388), VNTANST (SEQ ID NO: 40 of U.S. Pat. No. 9,475,845; herein SEQ ID NO: 2389), HGPMQKS (SEQ ID NO: 41 of U.S. Pat. No. 9,475,845; herein SEQ ID NO: 2390), PHKPPLA (SEQ ID NO: 42 of U.S. Pat. No. 9,475,845; herein SEQ ID NO: 2391), IKNNEMW (SEQ ID NO: 43 of U.S. Pat. No. 9,475,845; herein SEQ ID NO: 2392), RNLDTPM (SEQ ID NO: 44 of U.S. Pat. No. 9,475,845; herein SEQ ID NO: 2393), VDSHRQS (SEQ ID NO: 45 of U.S. Pat. No. 9,475,845; herein SEQ ID NO: 2394), YDSKTKT (SEQ ID NO: 46 of U.S. Pat. No. 9,475,845; herein SEQ ID NO: 2395), SQLPHQK (SEQ ID NO: 47 of U.S. Pat. No. 9,475,845; herein SEQ ID NO: 2396), STMQQNT (SEQ ID NO: 48 of U.S. Pat. No. 9,475,845; herein SEQ ID NO: 2397), TERYMTQ (SEQ ID NO: 49 of U.S. Pat. No. 9,475,845; herein SEQ ID NO: 2398), DASLSTS (SEQ ID NO: 51 of U.S. Pat. No. 9,475,845; herein SEQ ID NO: 2399), DLPNKKT (SEQ ID NO: 52 of U.S. Pat. No. 9,475,845; herein SEQ ID NO: 2400), DLTAARL (SEQ ID NO: 53 of U.S. Pat. No. 9,475,845; herein SEQ ID NO: 2401), EPHQFNY (SEQ ID NO: 54 of U.S. Pat. No. 9,475,845; herein SEQ ID NO: 2402), EPQSNHT (SEQ ID NO: 55 of U.S. Pat. No. 9,475,845; herein SEQ ID NO: 2403), MSSWPSQ (SEQ ID NO: 56 of U.S. Pat. No. 9,475,845; herein SEQ ID NO: 2404), NPKHNAT (SEQ ID NO: 57 of U.S. Pat. No. 9,475,845; herein SEQ ID NO: 2405), PDGMRTT (SEQ ID NO: 58 of U.S. Pat. No. 9,475,845; herein SEQ ID NO: 2406), PNNNKTT (SEQ ID NO: 59 of U.S. Pat. No. 9,475,845; herein SEQ ID NO: 2407), QSTTHDS (SEQ ID NO: 60 of U.S. Pat. No. 9,475,845; herein SEQ ID NO: 2408), TGSKQKQ (SEQ ID NO: 61 of U.S. Pat. No. 9,475,845; herein SEQ ID NO: 2409), SLKHQAL (SEQ ID NO: 62 of U.S. Pat. No. 9,475,845; herein SEQ ID NO: 2410), SPIDGEQ (SEQ ID NO: 63 of U.S. Pat. No. 9,475,845; herein SEQ ID NO: 2411), WIFPWIQL (SEQ ID NO: 64 and 112 of U.S. Pat. No. 9,475,845; herein SEQ ID NO: 2412), CDCRGDCFC (SEQ ID NO: 65 of U.S. Pat. No. 9,475,845; herein SEQ ID NO: 2413), CNGRC (SEQ ID NO: 66 of U.S. Pat. No. 9,475,845; herein SEQ ID NO: 2414), CPRECES (SEQ ID NO: 67 of U.S. Pat. No. 9,475,845; herein SEQ ID NO: 2415), CTTHWGFTLC (SEQ ID NO: 68 and 123 of U.S. Pat. No. 9,475,845; herein SEQ ID NO: 2416), CGRRAGGSC (SEQ ID NO: 69 of U.S. Pat. No. 9,475,845; herein SEQ ID NO: 2417), CKGGRAKDC (SEQ ID NO: 70 of U.S. Pat. No. 9,475,845; herein SEQ ID NO: 2418), CVPELGHEC (SEQ ID NO: 71 and 115 of U.S. Pat. No. 9,475,845; herein SEQ ID NO: 2419), CRRETAWAK (SEQ ID NO: 72 of U.S. Pat. No. 9,475,845; herein SEQ ID NO: 2420), VSWFSHRYSPFAVS (SEQ ID NO: 73 of U.S. Pat. No. 9,475,845; herein SEQ ID NO: 2421), GYRDGYAGPILYN (SEQ ID NO: 74 of U.S. Pat. No. 9,475,845; herein SEQ ID NO: 2422), XXXYXXX (SEQ ID NO: 75 of U.S. Pat. No. 9,475,845; herein SEQ ID NO: 2423), YXNW (SEQ ID NO: 76 of U.S. Pat. No. 9,475,845; herein SEQ ID NO: 2424), RPLPPLP (SEQ ID NO: 77 of U.S. Pat. No. 9,475,845; herein SEQ ID NO: 2425), APPLPPR (SEQ ID NO: 78 of U.S. Pat. No. 9,475,845; herein SEQ ID NO: 2426), DVFYPYPYASGS (SEQ ID NO: 79 of U.S. Pat. No. 9,475,845; herein SEQ ID NO: 2427), MYWYPY (SEQ ID NO: 80 of U.S. Pat. No. 9,475,845; herein SEQ ID NO: 2428), DITWDQLWDLMK (SEQ ID NO: 81 of U.S. Pat. No. 9,475,845; herein SEQ ID NO: 2429), CWDDXWLC (SEQ ID NO: 82 of U.S. Pat. No. 9,475,845; herein SEQ ID NO: 2430), EWCEYLGGYLRCYA (SEQ ID NO: 83 of U.S. Pat. No. 9,475,845; herein SEQ ID NO: 2431), YXCXXGPXTWXCXP (SEQ ID NO: 84 of U.S. Pat. No. 9,475,845; herein SEQ ID NO: 2432), IEGPTLRQWLAARA (SEQ ID NO: 85 of U.S. Pat. No. 9,475,845; herein SEQ ID NO: 2433), LWXXX (SEQ ID NO: 86 of U.S. Pat. No. 9,475,845; herein SEQ ID NO: 2434), XFXXYLW (SEQ ID NO: 87 of U.S. Pat. No. 9,475,845; herein SEQ ID NO: 2435), SSIISHFRWGLCD (SEQ ID NO: 88 of U.S. Pat. No. 9,475,845; herein SEQ ID NO: 2436), MSRPACPPNDKYE (SEQ ID NO: 89 of U.S. Pat. No. 9,475,845; herein SEQ ID NO: 2437), CLRSGRGC (SEQ ID NO: 90 of U.S. Pat. No. 9,475,845; herein SEQ ID NO: 2438), CHWMFSPWC (SEQ ID NO: 91 of U.S. Pat. No. 9,475,845; herein SEQ ID NO: 2439), WXXF (SEQ ID NO: 92 of U.S. Pat. No. 9,475,845; herein SEQ ID NO: 2440), CSSRLDAC (SEQ ID NO: 93 of U.S. Pat. No. 9,475,845; herein SEQ ID NO: 2441), CLPVASC (SEQ ID NO: 94 of U.S. Pat. No. 9,475,845; herein SEQ ID NO: 2442), CGFECVRQCPERC (SEQ ID NO: 95 of U.S. Pat. No. 9,475,845; herein SEQ ID NO: 2443), CVALCREACGEGC (SEQ ID NO: 96 of U.S. Pat. No. 9,475,845; herein SEQ ID NO: 2444), SWCEPGWCR (SEQ ID NO: 97 of U.S. Pat. No. 9,475,845; herein SEQ ID NO: 2445), YSGKWGW (SEQ ID NO: 98 of U.S. Pat. No. 9,475,845; herein SEQ ID NO: 2446), GLSGGRS (SEQ ID NO: 99 of U.S. Pat. No. 9,475,845; herein SEQ ID NO: 2447), LMLPRAD (SEQ ID NO: 100 of U.S. Pat. No. 9,475,845; herein SEQ ID NO: 2448), CSCFRDVCC (SEQ ID NO: 101 of U.S. Pat. No. 9,475,845; herein SEQ ID NO: 2449), CRDVVSVIC (SEQ ID NO: 102 of U.S. Pat. No. 9,475,845; herein SEQ ID NO: 2450), MARSGL (SEQ ID NO: 103 of U.S. Pat. No. 9,475,845; herein SEQ ID NO: 2451), MARAKE (SEQ ID NO: 104 of U.S. Pat. No. 9,475,845; herein SEQ ID NO: 2452), MSRTMS (SEQ ID NO: 105 of U.S. Pat. No. 9,475,845; herein SEQ ID NO: 2453), KCCYSL (SEQ ID NO: 106 of U.S. Pat. No. 9,475,845; herein SEQ ID NO: 2454), MYWGDSHWLQYWYE (SEQ ID NO: 107 of U.S. Pat. No. 9,475,845; herein SEQ ID NO: 2455), MQLPLAT (SEQ ID NO: 108 of U.S. Pat. No. 9,475,845; herein SEQ ID NO: 2456), EWLS (SEQ ID NO: 109 of U.S. Pat. No. 9,475,845; herein SEQ ID NO: 2457), SNEW (SEQ ID NO: 110 of U.S. Pat. No. 9,475,845; herein SEQ ID NO: 2458), TNYL (SEQ ID NO: 111 of U.S. Pat. No. 9,475,845; herein SEQ ID NO: 2459), WDLAWMFRLPVG (SEQ ID NO: 113 of U.S. Pat. No. 9,475,845; herein SEQ ID NO: 2460), CTVALPGGYVRVC (SEQ ID NO: 114 of U.S. Pat. No. 9,475,845; herein SEQ ID NO: 2461), CVAYCIEHHCWTC (SEQ ID NO: 116 of U.S. Pat. No. 9,475,845; herein SEQ ID NO: 2462), CVFAHNYDYLVC (SEQ ID NO: 117 of U.S. Pat. No. 9,475,845; herein SEQ ID NO: 2463), CVFTSNYAFC (SEQ ID NO: 118 of U.S. Pat. No. 9,475,845; herein SEQ ID NO: 2464), VHSPNKK (SEQ ID NO: 119 of U.S. Pat. No. 9,475,845; herein SEQ ID NO: 2465), CRGDGWC (SEQ ID NO: 120 of U.S. Pat. No. 9,475,845; herein SEQ ID NO: 2466), XRGCDX (SEQ ID NO: 121 of U.S. Pat. No. 9,475,845; herein SEQ ID NO: 2467), PXXX (SEQ ID NO: 122 of U.S. Pat. No. 9,475,845; herein SEQ ID NO: 2468), SGKGPRQITAL (SEQ ID NO: 124 of U.S. Pat. No. 9,475,845; herein SEQ ID NO: 2469), AAAAAAAAAXXXXX (SEQ ID NO: 125 of U.S. Pat. No. 9,475,845; herein SEQ ID NO: 2470), VYMSPF (SEQ ID NO: 126 of U.S. Pat. No. 9,475,845; herein SEQ ID NO: 2471), ATWLPPR (SEQ ID NO: 127 of U.S. Pat. No. 9,475,845; herein SEQ ID NO: 2472), HTMYYHHYQHHL (SEQ ID NO: 128 of U.S. Pat. No. 9,475,845; herein SEQ ID NO: 2473), SEVGCRAGPLQWLCEKYFG (SEQ ID NO: 129 of U.S. Pat. No. 9,475,845; herein SEQ ID NO: 2474), CGLLPVGRPDRNVWRWLC (SEQ ID NO: 130 of U.S. Pat. No. 9,475,845; herein SEQ ID NO: 2475), CKGQCDRFKGLPWEC (SEQ ID NO: 131 of U.S. Pat. No. 9,475,845; herein SEQ ID NO: 2476), SGRSA (SEQ ID NO: 132 of U.S. Pat. No. 9,475,845; herein SEQ ID NO: 2477), WGFP (SEQ ID NO: 133 of U.S. Pat. No. 9,475,845; herein SEQ ID NO: 2478), AEPMPHSLNFSQYLWYT (SEQ ID NO: 134 of U.S. Pat. No. 9,475,845; herein SEQ ID NO: 2479), WAYXSP (SEQ ID NO: 135 of U.S. Pat. No. 9,475,845; herein SEQ ID NO: 2480), IELLQAR (SEQ ID NO: 136 of U.S. Pat. No. 9,475,845; herein SEQ ID NO: 2481), AYTKCSRQWRTCMTTH (SEQ ID NO: 137 of U.S. Pat. No. 9,475,845; herein SEQ ID NO: 2482), PQNSKIPGPTFLDPH (SEQ ID NO: 138 of U.S. Pat. No. 9,475,845; herein SEQ ID NO: 2483), SMEPALPDWWWKMFK (SEQ ID NO: 139 of U.S. Pat. No. 9,475,845; herein SEQ ID NO: 2484), ANTPCGPYTHDCPVKR (SEQ ID NO: 140 of U.S. Pat. No. 9,475,845; herein SEQ ID NO: 2485), TACHQHVRMVRP (SEQ ID NO: 141 of U.S. Pat. No. 9,475,845; herein SEQ ID NO: 2486), VPWMEPAYQRFL (SEQ ID NO: 142 of U.S. Pat. No. 9,475,845; herein SEQ ID NO: 2487), DPRATPGS (SEQ ID NO: 143 of U.S. Pat. No. 9,475,845; herein SEQ ID NO: 2488), FRPNRAQDYNTN (SEQ ID NO: 144 of U.S. Pat. No. 9,475,845; herein SEQ ID NO: 2489), CTKNSYLMC (SEQ ID NO: 145 of U.S. Pat. No. 9,475,845; herein SEQ ID NO: 2490), CXXTXXXGXGC (SEQ ID NO: 146 of U.S. Pat. No. 9,475,845; herein SEQ ID NO: 2491), CPIEDRPMC (SEQ ID NO: 147 of U.S. Pat. No. 9,475,845; herein SEQ ID NO: 2492), HEWSYLAPYPWF (SEQ ID NO: 148 of U.S. Pat. No. 9,475,845; herein SEQ ID NO: 2493), MCPKHPLGC (SEQ ID NO: 149 of U.S. Pat. No. 9,475,845; herein SEQ ID NO: 2494), RMWPSSTVNLSAGRR (SEQ ID NO: 150 of U.S. Pat. No. 9,475,845; herein SEQ ID NO: 2495), SAKTAVSQRVWLPSHRGGEP (SEQ ID NO: 151 of U.S. Pat. No. 9,475,845; herein SEQ ID NO: 2496), KSREHVNNSACPSKRITAAL (SEQ ID NO: 152 of U.S. Pat. No. 9,475,845; herein SEQ ID NO: 2497), EGFR (SEQ ID NO: 153 of U.S. Pat. No. 9,475,845; herein SEQ ID NO: 2498), AGLGVR (SEQ ID NO: 154 of U.S. Pat. No. 9,475,845; herein SEQ ID NO: 2499), GTRQGHTMRLGVSDG (SEQ ID NO: 155 of U.S. Pat. No. 9,475,845; herein SEQ ID NO: 2500), IAGLATPGWSHWLAL (SEQ ID NO: 156 of U.S. Pat. No. 9,475,845; herein SEQ ID NO: 2501), SMSIARL (SEQ ID NO: 157 of U.S. Pat. No. 9,475,845; herein SEQ ID NO: 2502), HTFEPGV (SEQ ID NO: 158 of U.S. Pat. No. 9,475,845; herein SEQ ID NO: 2503), NTSLKRISNKRIRRK (SEQ ID NO: 159 of U.S. Pat. No. 9,475,845; herein SEQ ID NO: 2504), LRIKRKRRKRKKTRK (SEQ ID NO: 160 of U.S. Pat. No. 9,475,845; herein SEQ ID NO: 2505), GGG, GFS, LWS, EGG, LLV, LSP, LBS, AGG, GRR, GGH and GTV.

In one embodiment, the AAV serotype may be, or may have a sequence as described in United States Publication No. US 20160369298, the contents of which are herein incorporated by reference in their entirety, such as, but not limited to, site-specific mutated capsid protein of AAV2 (SEQ ID NO: 97 of US 20160369298; herein SEQ ID NO: 2506) or variants thereof, wherein the specific site is at least one site selected from sites R447, G453, S578, N587, N587+1, S662 of VP1 or fragment thereof.

Further, any of the mutated sequences described in US 20160369298, may be or may have, but not limited to, any of the following sequences SDSGASN (SEQ ID NO: 1 and SEQ ID NO: 231 of US20160369298; herein SEQ ID NO: 2507), SPSGASN (SEQ ID NO: 2 of US20160369298; herein SEQ ID NO: 2508), SHSGASN (SEQ ID NO: 3 of US20160369298; herein SEQ ID NO: 2509), SRSGASN (SEQ ID NO: 4 of US20160369298; herein SEQ ID NO: 2510), SKSGASN (SEQ ID NO: 5 of US20160369298; herein SEQ ID NO: 2511), SNSGASN (SEQ ID NO: 6 of US20160369298; herein SEQ ID NO: 2512), SGSGASN (SEQ ID NO: 7 of US20160369298; herein SEQ ID NO: 2513), SASGASN (SEQ ID NO: 8, 175, and 221 of US20160369298; herein SEQ ID NO: 2514), SESGTSN (SEQ ID NO: 9 of US20160369298; herein SEQ ID NO: 2515), STTGGSN (SEQ ID NO: 10 of US20160369298; herein SEQ ID NO: 2516), SSAGSTN (SEQ ID NO: 11 of US20160369298; herein SEQ ID NO: 2517), NNDSQA (SEQ ID NO: 12 of US20160369298; herein SEQ ID NO: 2518), NNRNQA (SEQ ID NO: 13 of US20160369298; herein SEQ ID NO: 2519), NNNKQA (SEQ ID NO: 14 of US20160369298; herein SEQ ID NO: 2520), NAKRQA (SEQ ID NO: 15 of US20160369298; herein SEQ ID NO: 2521), NDEHQA (SEQ ID NO: 16 of US20160369298; herein SEQ ID NO: 2522), NTSQKA (SEQ ID NO: 17 of US20160369298; herein SEQ ID NO: 2523), YYLSRTNTPSGTDTQSRLVFSQAGA (SEQ ID NO: 18 of US20160369298; herein SEQ ID NO: 2524), YYLSRTNTDSGTETQSGLDFSQAGA (SEQ ID NO: 19 of US20160369298; herein SEQ ID NO: 2525), YYLSRTNTESGTPTQSALEFSQAGA (SEQ ID NO: 20 of US20160369298; herein SEQ ID NO: 2526), YYLSRTNTHSGTHTQSPLHFSQAGA (SEQ ID NO: 21 of US20160369298; herein SEQ ID NO: 2527), YYLSRTNTSSGTITISHLIFSQAGA (SEQ ID NO: 22 of US20160369298; herein SEQ ID NO: 2528), YYLSRTNTRSGIMTKSSLMFSQAGA (SEQ ID NO: 23 of US20160369298; herein SEQ ID NO: 2529), YYLSRTNTKSGRKTLSNLSFSQAGA (SEQ ID NO: 24 of US20160369298; herein SEQ ID NO: 2530), YYLSRTNDGSGPVTPSKLRFSQRGA (SEQ ID NO: 25 of US20160369298; herein SEQ ID NO: 2531), YYLSRTNAASGHATHSDLKFSQPGA (SEQ ID NO: 26 of US20160369298; herein SEQ ID NO: 2532), YYLSRTNGQAGSLTMSELGFSQVGA (SEQ ID NO: 27 of US20160369298; herein SEQ ID NO: 2533), YYLSRTNSTGGNQTTSQLLFSQLSA (SEQ ID NO: 28 of US20160369298; herein SEQ ID NO: 2534), YFLSRTNNNTGLNTNSTLNFSQGRA (SEQ ID NO: 29 of US20160369298; herein SEQ ID NO: 2535), SKTGADNNNSEYSWTG (SEQ ID NO: 30 of US20160369298; herein SEQ ID NO: 2536), SKTDADNNNSEYSWTG (SEQ ID NO: 31 of US20160369298; herein SEQ ID NO: 2537), SKTEADNNNSEYSWTG (SEQ ID NO: 32 of US20160369298; herein SEQ ID NO: 2538), SKTPADNNNSEYSWTG (SEQ ID NO: 33 of US20160369298; herein SEQ ID NO: 2539), SKTHADNNNSEYSWTG (SEQ ID NO: 34 of US20160369298; herein SEQ ID NO: 2540), SKTQADNNNSEYSWTG (SEQ ID NO: 35 of US20160369298; herein SEQ ID NO: 2541), SKTIADNNNSEYSWTG (SEQ ID NO: 36 of US20160369298; herein SEQ ID NO: 2542), SKTMADNNNSEYSWTG (SEQ ID NO: 37 of US20160369298; herein SEQ ID NO: 2543), SKTRADNNNSEYSWTG (SEQ ID NO: 38 of US20160369298; herein SEQ ID NO: 2544), SKTNADNNNSEYSWTG (SEQ ID NO: 39 of US20160369298; herein SEQ ID NO: 2545), SKTVGRNNNSEYSWTG (SEQ ID NO: 40 of US20160369298; herein SEQ ID NO: 2546), SKTADRNNNSEYSWTG (SEQ ID NO: 41 of US20160369298; herein SEQ ID NO: 2547), SKKLSQNNNSKYSWQG (SEQ ID NO: 42 of US20160369298; herein SEQ ID NO: 2548), SKPTTGNNNSDYSWPG (SEQ ID NO: 43 of US20160369298; herein SEQ ID NO: 2549), STQKNENNNSNYSWPG (SEQ ID NO: 44 of US20160369298; herein SEQ ID NO: 2550), HKDDEGKF (SEQ ID NO: 45 of US20160369298; herein SEQ ID NO: 2551), HKDDNRKF (SEQ ID NO: 46 of US20160369298; herein SEQ ID NO: 2552), HKDDTNKF (SEQ ID NO: 47 of US20160369298; herein SEQ ID NO: 2553), HEDSDKNF (SEQ ID NO: 48 of US20160369298; herein SEQ ID NO: 2554), HRDGADSF (SEQ ID NO: 49 of US20160369298; herein SEQ ID NO: 2555), HGDNKSRF (SEQ ID NO: 50 of US20160369298; herein SEQ ID NO: 2556), KQGSEKTNVDFEEV (SEQ ID NO: 51 of US20160369298; herein SEQ ID NO: 2557), KQGSEKTNVDSEEV (SEQ ID NO: 52 of US20160369298; herein SEQ ID NO: 2558), KQGSEKTNVDVEEV (SEQ ID NO: 53 of US20160369298; herein SEQ ID NO: 2559), KQGSDKTNVDDAGV (SEQ ID NO: 54 of US20160369298; herein SEQ ID NO: 2560), KQGSSKTNVDPREV (SEQ ID NO: 55 of US20160369298; herein SEQ ID NO: 2561), KQGSRKTNVDHKQV (SEQ ID NO: 56 of US20160369298; herein SEQ ID NO: 2562), KQGSKGGNVDTNRV (SEQ ID NO: 57 of US20160369298; herein SEQ ID NO: 2563), KQGSGEANVDNGDV (SEQ ID NO: 58 of US20160369298; herein SEQ ID NO: 2564), KQDAAADNIDYDHV (SEQ ID NO: 59 of US20160369298; herein SEQ ID NO: 2565), KQSGTRSNAAASSV (SEQ ID NO: 60 of US20160369298; herein SEQ ID NO: 2566), KENTNTNDTELTNV (SEQ ID NO: 61 of US20160369298; herein SEQ ID NO: 2567), QRGNNVAATADVNT (SEQ ID NO: 62 of US20160369298; herein SEQ ID NO: 2568), QRGNNEAATADVNT (SEQ ID NO: 63 of US20160369298; herein SEQ ID NO: 2569), QRGNNPAATADVNT (SEQ ID NO: 64 of US20160369298; herein SEQ ID NO: 2570), QRGNNHAATADVNT (SEQ ID NO: 65 of US20160369298; herein SEQ ID NO: 2571), QEENNIAATPGVNT (SEQ ID NO: 66 of US20160369298; herein SEQ ID NO: 2572), QPPNNMAATHEVNT (SEQ ID NO: 67 of US20160369298; herein SEQ ID NO: 2573), QHHNNSAATTIVNT (SEQ ID NO: 68 of US20160369298; herein SEQ ID NO: 2574), QTTNNRAAFNMVET (SEQ ID NO: 69 of US20160369298; herein SEQ ID NO: 2575), QKKNNNAASKKVAT (SEQ ID NO: 70 of US20160369298; herein SEQ ID NO: 2576), QGGNNKAADDAVKT (SEQ ID NO: 71 of US20160369298; herein SEQ ID NO: 2577), QAAKGGAADDAVKT (SEQ ID NO: 72 of US20160369298; herein SEQ ID NO: 2578), QDDRAAAANESVDT (SEQ ID NO: 73 of US20160369298; herein SEQ ID NO: 2579), QQQHDDAAYQRVHT (SEQ ID NO: 74 of US20160369298; herein SEQ ID NO: 2580), QSSSSLAAVSTVQT (SEQ ID NO: 75 of US20160369298; herein SEQ ID NO: 2581), QNNQTTAAIRNVTT (SEQ ID NO: 76 of US20160369298; herein SEQ ID NO: 2582), NYNKKSDNVDFT (SEQ ID NO: 77 of US20160369298; herein SEQ ID NO: 2583), NYNKKSENVDFT (SEQ ID NO: 78 of US20160369298; herein SEQ ID NO: 2584), NYNKKSLNVDFT (SEQ ID NO: 79 of US20160369298; herein SEQ ID NO: 2585), NYNKKSPNVDFT (SEQ ID NO: 80 of US20160369298; herein SEQ ID NO: 2586), NYSKKSHCVDFT (SEQ ID NO: 81 of US20160369298; herein SEQ ID NO: 2587), NYRKTIYVDFT (SEQ ID NO: 82 of US20160369298; herein SEQ ID NO: 2588), NYKEKKDVHFT (SEQ ID NO: 83 of US20160369298; herein SEQ ID NO: 2589), NYGHRAIVQFT (SEQ ID NO: 84 of US20160369298; herein SEQ ID NO: 2590), NYANHQFVVCT (SEQ ID NO: 85 of US20160369298; herein SEQ ID NO: 2591), NYDDDPTGVLLT (SEQ ID NO: 86 of US20160369298; herein SEQ ID NO: 2592), NYDDPTGVLLT (SEQ ID NO: 87 of US20160369298; herein SEQ ID NO: 2593), NFEQQNSVEWT (SEQ ID NO: 88 of US20160369298; herein SEQ ID NO: 2594), SQSGASN (SEQ ID NO: 89 and SEQ ID NO: 241 of US20160369298; herein SEQ ID NO: 2595), NNGSQA (SEQ ID NO: 90 of US20160369298; herein SEQ ID NO: 2596), YYLSRTNTPSGTTTWSRLQFSQAGA (SEQ ID NO: 91 of US20160369298; herein SEQ ID NO: 2597), SKTSADNNNSEYSWTG (SEQ ID NO: 92 of US20160369298; herein SEQ ID NO: 2598), HKDDEEKF (SEQ ID NO: 93, 209, 214, 219, 224, 234, 239, and 244 of US20160369298; herein SEQ ID NO: 2599), KQGSEKTNVDIEEV (SEQ ID NO: 94 of US20160369298; herein SEQ ID NO: 2600), QRGNNQAATADVNT (SEQ ID NO: 95 of US20160369298; herein SEQ ID NO: 2601), NYNKKSVNVDFT (SEQ ID NO: 96 of US20160369298; herein SEQ ID NO: 2602), SQSGASNYNTPSGTTTQSRLQFSTSADNNNSEYSWTGATKYH (SEQ ID NO: 106 of US20160369298; herein SEQ ID NO: 2603), SASGASNFNSEGGSLTQSSLGFSTDGENNNSDFSWTGATKYH (SEQ ID NO: 107 of US20160369298; herein SEQ ID NO: 2604), SQSGASNYNTPSGTTTQSRLQFSTDGENNNSDFSWTGATKYH (SEQ ID NO: 108 of US20160369298; herein SEQ ID NO: 2605), SASGASNYNTPSGTTTQSRLQFSTSADNNNSEFSWPGATTYH (SEQ ID NO: 109 of US20160369298; herein SEQ ID NO: 2606), SQSGASNFNSEGGSLTQSSLGFSTDGENNNSDFSWTGATKYH (SEQ ID NO: 110 of US20160369298; herein SEQ ID NO: 2607), SASGASNYNTPSGSLTQSSLGFSTDGENNNSDFSWTGATKYH (SEQ ID NO: 111 of US20160369298; herein SEQ ID NO: 2608), SQSGASNYNTPSGTTTQSRLQFSTSADNNNSDFSWTGATKYH (SEQ ID NO: 112 of US20160369298; herein SEQ ID NO: 2609), SGAGASNFNSEGGSLTQSSLGFSTDGENNNSDFSWTGATKYH (SEQ ID NO: 113 of US20160369298; herein SEQ ID NO: 2610), SGAGASN (SEQ ID NO: 176 of US20160369298; herein SEQ ID NO: 2611), NSEGGSLTQSSLGFS (SEQ ID NO: 177, 185, 193 and 202 of US20160369298; herein SEQ ID NO: 2612), TDGENNNSDFS (SEQ ID NO: 178 of US20160369298; herein SEQ ID NO: 2613), SEFSWPGATT (SEQ ID NO: 179 of US20160369298; herein SEQ ID NO: 2614), TSADNNNSDFSWT (SEQ ID NO: 180 of US20160369298; herein SEQ ID NO: 2615), SQSGASNY (SEQ ID NO: 181, 187, and 198 of US20160369298; herein SEQ ID NO: 2616), NTPSGTTTQSRLQFS (SEQ ID NO: 182, 188, 191, and 199 of US20160369298; herein SEQ ID NO: 2617), TSADNNNSEYSWTGATKYH (SEQ ID NO: 183 of US20160369298; herein SEQ ID NO: 2618), SASGASNF (SEQ ID NO: 184 of US20160369298; herein SEQ ID NO: 2619), TDGENNNSDFSWTGATKYH (SEQ ID NO: 186, 189, 194, 197, and 203 of US20160369298; herein SEQ ID NO: 2620), SASGASNY (SEQ ID NO: 190 and SEQ ID NO: 195 of US20160369298; herein SEQ ID NO: 2621), TSADNNNSEFSWPGATTYH (SEQ ID NO: 192 of US20160369298; herein SEQ ID NO: 2622), NTPSGSLTQSSLGFS (SEQ ID NO: 196 of US20160369298; herein SEQ ID NO: 2623), TSADNNNSDFSWTGATKYH (SEQ ID NO: 200 of US20160369298; herein SEQ ID NO: 2624), SGAGASNF (SEQ ID NO: 201 of US20160369298; herein SEQ ID NO: 2625), CTCCAGVVSVVSMRSRVCVNSGCAGCTDHCVVSRNSGTCVMSACACAA (SEQ ID NO: 204 of US20160369298; herein SEQ ID NO: 2626), CTCCAGAGAGGCAACAGACAAGCAGCTACCGCAGATGTCAACACACAA (SEQ ID NO: 205 of US20160369298; herein SEQ ID NO: 2627), SAAGASN (SEQ ID NO: 206 of US20160369298; herein SEQ ID NO: 2628), YFLSRTNTESGSTTQSTLRFSQAG (SEQ ID NO: 207 of US20160369298; herein SEQ ID NO: 2629), SKTSADNNNSDFS (SEQ ID NO: 208, 228, and 253 of US20160369298; herein SEQ ID NO: 2630), KQGSEKTDVDIDKV (SEQ ID NO: 210 of US20160369298; herein SEQ ID NO: 2631), STAGASN (SEQ ID NO: 211 of US20160369298; herein SEQ ID NO: 2632), YFLSRTNTTSGIETQSTLRFSQAG (SEQ ID NO: 212 and SEQ ID NO: 247 of US20160369298; herein SEQ ID NO: 2633), SKTDGENNNSDFS (SEQ ID NO: 213 and SEQ ID NO: 248 of US20160369298; herein SEQ ID NO: 2634), KQGAAADDVEIDGV (SEQ ID NO: 215 and SEQ ID NO: 250 of US20160369298; herein SEQ ID NO: 2635), SEAGASN (SEQ ID NO: 216 of US20160369298; herein SEQ ID NO: 2636), YYLSRTNTPSGTTTQSRLQFSQAG (SEQ ID NO: 217, 232 and 242 of US20160369298; herein SEQ ID NO: 2637), SKTSADNNNSEYS (SEQ ID NO: 218, 233, 238, and 243 of US20160369298; herein SEQ ID NO: 2638), KQGSEKTNVDIEKV (SEQ ID NO: 220, 225 and 245 of US20160369298; herein SEQ ID NO: 2639), YFLSRTNDASGSDTKSTLLFSQAG (SEQ ID NO: 222 of US20160369298; herein SEQ ID NO: 2640), STTPSENNNSEYS (SEQ ID NO: 223 of US20160369298; herein SEQ ID NO: 2641), SAAGATN (SEQ ID NO: 226 and SEQ ID NO: 251 of US20160369298; herein SEQ ID NO: 2642), YFLSRTNGEAGSATLSELRFSQAG (SEQ ID NO: 227 of US20160369298; herein SEQ ID NO: 2643), HGDDADRF (SEQ ID NO: 229 and SEQ ID NO: 254 of US20160369298; herein SEQ ID NO: 2644), KQGAEKSDVEVDRV (SEQ ID NO: 230 and SEQ ID NO: 255 of US20160369298; herein SEQ ID NO: 2645), KQDSGGDNIDIDQV (SEQ ID NO: 235 of US20160369298; herein SEQ ID NO: 2646), SDAGASN (SEQ ID NO: 236 of US20160369298; herein SEQ ID NO: 2647), YFLSRTNTEGGHDTQSTLRFSQAG (SEQ ID NO: 237 of US20160369298; herein SEQ ID NO: 2648), KEDGGGSDVAIDEV (SEQ ID NO: 240 of US20160369298; herein SEQ ID NO: 2649), SNAGASN (SEQ ID NO: 246 of US20160369298; herein SEQ ID NO: 2650), and YFLSRTNGEAGSATLSELRFSQPG (SEQ ID NO: 252 of US20160369298; herein SEQ ID NO: 2651). Non-limiting examples of nucleotide sequences that may encode the amino acid mutated sites include the following, AGCVVMDCAGGARSCASCAAC (SEQ ID NO: 97 of US20160369298; herein SEQ ID NO: 2652), AACRACRRSMRSMAGGCA (SEQ ID NO: 98 of US20160369298; herein SEQ ID NO: 2653), CACRRGGACRRCRMSRRSARSTTT (SEQ ID NO: 99 of US20160369298; herein SEQ ID NO: 2654), TATTTCTTGAGCAGAACAAACRVCVVSRSCGGAMNCVHSACGMHSTCAVVSCTTVDS TTTTCTCAGSBCRGSGCG (SEQ ID NO: 100 of US20160369298; herein SEQ ID NO: 2655), TCAAMAMMAVNSRVCSRSAACAACAACAGTRASTTCTCGTGGMMAGGA (SEQ ID NO: 101 of US20160369298; herein SEQ ID NO: 2656), AAGSAARRCRSCRVSRVARVCRATRYCGMSNHCRVMVRSGTC (SEQ ID NO: 102 of US20160369298; herein SEQ ID NO: 2657), CAGVVSVVSMRSRVCVNSGCAGCTDHCVVSRNSGTCVMSACA (SEQ ID NO: 103 of US20160369298; herein SEQ ID NO: 2658), AACTWCRVSVASMVSVHSDDTGTGSWSTKSACT (SEQ ID NO: 104 of US20160369298; herein SEQ ID NO: 2659), TTGTTGAACATCACCACGTGACGCACGTTC (SEQ ID NO: 256 of US20160369298; herein SEQ ID NO: 2660), TCCCCGTGGTTCTACTACATAATGTGGCCG (SEQ ID NO: 257 of US20160369298; herein SEQ ID NO: 2661), TTCCACACTCCGTTTTGGATAATGTTGAAC (SEQ ID NO: 258 of US20160369298; herein SEQ ID NO: 2662), AGGGACATCCCCAGCTCCATGCTGTGGTCG (SEQ ID NO: 259 of US20160369298; herein SEQ ID NO: 2663), AGGGACAACCCCTCCGACTCGCCCTAATCC (SEQ ID NO: 260 of US20160369298; herein SEQ ID NO: 2664), TCCTAGTAGAAGACACCCTCTCACTGCCCG (SEQ ID NO: 261 of US20160369298; herein SEQ ID NO: 2665), AGTACCATGTACACCCACTCTCCCAGTGCC (SEQ ID NO: 262 of US20160369298; herein SEQ ID NO: 2666), ATATGGACGTTCATGCTGATCACCATACCG (SEQ ID NO: 263 of US20160369298; herein SEQ ID NO: 2667), AGCAGGAGCTCCTTGGCCTCAGCGTGCGAG (SEQ ID NO: 264 of US20160369298; herein SEQ ID NO: 2668), ACAAGCAGCTTCACTATGACAACCACTGAC (SEQ ID NO: 265 of US20160369298; herein SEQ ID NO: 2669), CAGCCTAGGAACTGGCTTCCTGGACCCTGTTACCGCCAGCAGAGAGTCTCAAMAMM AVNSRVCSRSAACAACAACAGTRASTTCTCCTGGMMAGGAGCTACCAAGTACCACC TCAATGGCAGAGACTCTCTGGTGAATCCCGGACCAGCTATGGCAAGCCACRRGGAC RRCRMSRRSARSTTTTTTCCTCAGAGCGGGGTTCTCATCTTTGGGAAGSAARRCRSCR VSRVARVCRATRYCGMSNHCRVMVRSGTCATGATTACAGACGAAGAGGAGATCTGG AC (SEQ ID NO: 266 of US20160369298; herein SEQ ID NO: 2670), TGGGACAATGGCGGTCGTCTCTCAGAGTTKTKKT (SEQ ID NO: 267 of US20160369298; herein SEQ ID NO: 2671), AGAGGACCKKTCCTCGATGGTTCATGGTGGAGTTA (SEQ ID NO: 268 of US20160369298; herein SEQ ID NO: 2672), CCACTTAGGGCCTGGTCGATACCGTTCGGTG (SEQ ID NO: 269 of US20160369298; herein SEQ ID NO: 2673), and TCTCGCCCCAAGAGTAGAAACCCTTCSTTYYG (SEQ ID NO: 270 of US20160369298; herein SEQ ID NO: 2674).

In some embodiments, the AAV serotype may comprise an ocular cell targeting peptide as described in International Patent Publication WO2016134375, the contents of which are herein incorporated by reference in their entirety, such as, but not limited to SEQ ID NO: 9, and SEQ ID NO:10 of WO2016134375. Further, any of the ocular cell targeting peptides or amino acids described in WO2016134375, may be inserted into any parent AAV serotype, such as, but not limited to, AAV2 (SEQ ID NO:8 of WO2016134375; herein SEQ ID NO: 2675), or AAV9 (SEQ ID NO: 11 of WO2016134375; herein SEQ ID NO: 2676). In some embodiments, modifications, such as insertions are made in AAV2 proteins at P34-A35, T138-A139, A139-P140, G453-T454, N587-R588, and/or R588-Q589. In certain embodiments, insertions are made at D384, G385, 1560, T561, N562, E563, E564, E565, N704, and/or Y705 of AAV9. The ocular cell targeting peptide may be, but is not limited to, any of the following amino acid sequences, GSTPPPM (SEQ ID NO: 1 of WO2016134375; herein SEQ ID NO: 2677), or GETRAPL (SEQ ID NO: 4 of WO2016134375; herein SEQ ID NO: 2678).

In some embodiments, the AAV serotype may be modified as described in the United States Publication US 20170145405 the contents of which are herein incorporated by reference in their entirety. AAV serotypes may include, modified AAV2 (e.g., modifications at Y444F, Y500F, Y730F and/or S662V), modified AAV3 (e.g., modifications at Y705F, Y731F and/or T492V), and modified AAV6 (e.g., modifications at S663V and/or T492V).

In some embodiments, the AAV serotype may be modified as described in the International Publication WO2017083722 the contents of which are herein incorporated by reference in their entirety. AAV serotypes may include, AAV1 (Y705+731F+T492V), AAV2 (Y444+500+730F+T491V), AAV3 (Y705+731F), AAV5, AAV 5 (Y436+693+719F), AAV6 (VP3 variant Y705F/Y731F/T492V), AAV8 (Y733F), AAV9, AAV9 (VP3 variant Y731F), and AAV10 (Y733F).

In some embodiments, the AAV serotype may comprise, as described in International Patent Publication WO2017015102, the contents of which are herein incorporated by reference in their entirety, an engineered epitope comprising the amino acids SPAKFA (SEQ ID NO: 24 of WO2017015102; herein SEQ ID NO: 2679) or NKDKLN (SEQ ID NO:2 of WO2017015102; herein SEQ ID NO: 2680). The epitope may be inserted in the region of amino acids 665 to 670 based on the numbering of the VP1 capsid of AAV8 (SEQ ID NO:3 of WO2017015102) and/or residues 664 to 668 of AAV3B (SEQ ID NO:3).

In one embodiment, the AAV serotype may be, or may have a sequence as described in International Patent Publication WO2017058892, the contents of which are herein incorporated by reference in their entirety, such as, but not limited to, AAV variants with capsid proteins that may comprise a substitution at one or more (e.g., 2, 3, 4, 5, 6, or 7) of amino acid residues 262-268, 370-379, 451-459, 472-473, 493-500, 528-534, 547-552, 588-597, 709-710, 716-722 of AAV1, in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV. The amino acid substitution may be, but is not limited to, any of the amino acid sequences described in WO2017058892. In one embodiment, the AAV may comprise an amino acid substitution at residues 256L, 258K, 259Q, 261S, 263A, 264S, 265T, 266G, 272H, 385S, 386Q, S472R, V473D, N500E 547S, 709A, 710N, 716D, 717N, 718N, 720L, A456T, Q457T, N458Q, K459S, T492S, K493A, S586R, S587G, S588N, T589R and/or 722T of AAV1 (SEQ ID NO: 1 of WO2017058892) in any combination, 244N, 246Q, 248R, 249E, 2501, 251K, 252S, 253G, 254S, 255V, 256D, 263Y, 377E, 378N, 453L, 456R, 532Q, 533P, 535N, 536P, 537G, 538T, 539T, 540A, 541T, 542Y, 543L, 546N, 653V, 654P, 656S, 697Q, 698F, 704D, 705S, 706T, 707G, 708E, 709Y and/or 710R of AAV5 (SEQ ID NO:5 of WO2017058892) in any combination, 248R, 316V, 317Q, 318D, 319S, 443N, 530N, 5315, 532Q 533P, 534A, 535N, 540A, 541 T, 542Y, 543L, 545G, 546N, 697Q, 704D, 706T, 708E, 709Y and/or 710R of AAV5 (SEQ ID NO: 5 of WO2017058892) in any combination, 264S, 266G, 269N, 272H, 457Q, 588S and/or 589I of AAV6 (SEQ ID NO:6 WO2017058892) in any combination, 457T, 459N, 496G, 499N, 500N, 589Q, 590N and/or 592A of AAV8 (SEQ ID NO: 8 WO2017058892) in any combination, 451I, 452N, 453G, 454S, 455G, 456Q, 457N and/or 458Q of AAV9 (SEQ ID NO: 9 WO2017058892) in any combination.

In some embodiments, the AAV may include a sequence of amino acids at positions 155, 156 and 157 of VP1 or at positions 17, 18, 19 and 20 of VP2, as described in International Publication No. WO 2017066764, the contents of which are herein incorporated by reference in their entirety. The sequences of amino acid may be, but not limited to, N-S-S, S-X-S, S-S-Y, N-X-S, N-S-Y, S-X-Y and N-X-Y, where N, X and Y are, but not limited to, independently non-serine, or non-threonine amino acids, wherein the AAV may be, but not limited to AAV1, AAV2, AAV3, AAV4, AAV5, AAV6, AAV7, AAV8, AAV9, AAV10, AAV11 and AAV12. In some embodiments, the AAV may include a deletion of at least one amino acid at positions 156, 157 or 158 of VP1 or at positions 19, 20 or 21 of VP2, wherein the AAV may be, but not limited to AAV1, AAV2, AAV3, AAV4, AAV5, AAV6, AAV7, AAV8, AAV9, AAV10, AAV11 and AAV12.

In one embodiment, peptides for inclusion in an AAV serotype may be identified using the methods described by Hui et al. (Molecular Therapy—Methods & Clinical Development (2015) 2, 15029 doi:10.1038/mtm.2015.29; the contents of which are herein incorporated by reference in its entirety). As a non-limiting example, the procedure includes isolating human splenocytes, restimulating the splenocytes in vitro using individual peptides spanning the amino acid sequence of the AAV capsid protein, IFN-gamma ELISpot with the individual peptides used for the in vitro restimulation, bioinformatics analysis to determine the HLA restriction of 15-mers identified by IFN-gamma ELISpot, identification of candidate reactive 9-mer epitopes for a given HLA allele, synthesis candidate 9-mers, second IFN-gamma ELISpot screening of splenocytes from subjects carrying the HLA alleles to which identified AAV epitopes are predicted to bind, determine the AAV capsid-reactive CD8+ T cell epitopes and determine the frequency of subjects reacting to a given AAV epitope.

In one embodiment, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In one embodiment, AAV serotypes generated in this manner have improved CNS transduction and/or neuronal and astrocytic tropism, as compared to other AAV serotypes. As non-limiting examples, the AAV serotype may be PHP.B, PHP.B2, PHP.B3, PHP.A, G2A12, G2A15. In one embodiment, these AAV serotypes may be AAV9 (SEQ ID NO: 126 and 127) derivatives with a 7-amino acid insert between amino acids 588-589. Non-limiting examples of these 7-amino acid inserts include TLAVPFK (SEQ ID NO: 873), SVSKPFL (SEQ ID NO: 1249), FTLTTPK (SEQ ID NO: 882), YTLSQGW (SEQ ID NO: 888), QAVRTSL (SEQ ID NO: 914) and/or LAKERLS (SEQ ID NO: 915).

In one embodiment, the AAV serotype may be as described in Jackson et al (Frontiers in Molecular Neuroscience 9:154 (2016)), the contents of which are herein incorporated by reference in their entirety. In some embodiments, the AAV serotype is PHP.B or AAV9. In some embodiments, the AAV serotype is paired with a synapsin promoter to enhance neuronal transduction, as compared to when more ubiquitous promoters are used (i.e., CBA or CMV).

In one embodiment, peptides for inclusion in an AAV serotype may be identified by isolating human splenocytes, restimulating the splenocytes in vitro using individual peptides spanning the amino acid sequence of the AAV capsid protein, IFN-gamma ELISpot with the individual peptides used for the in vitro restimulation, bioinformatics analysis to determine the given allele restriction of 15-mers identified by IFN-gamma ELISpot, identification of candidate reactive 9-mer epitopes for a given allele, synthesis candidate 9-mers, second IFN-gamma ELISpot screening of splenocytes from subjects carrying the specific alleles to which identified AAV epitopes are predicted to bind, determine the AAV capsid-reactive CD8+ T cell epitopes and determine the frequency of subjects reacting to a given AAV epitope.

AAV particles comprising a modulatory polynucleotide encoding the siRNA molecules may be prepared or derived from various serotypes of AAVs, including, but not limited to, AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ8 and AAV-DJ. In some cases, different serotypes of AAVs may be mixed together or with other types of viruses to produce chimeric AAV particles. As a non-limiting example, the AAV particle is derived from the AAV9 serotype.

### Viral Genome

In one embodiment, as shown in an AAV particle comprises a viral genome with a payload region.

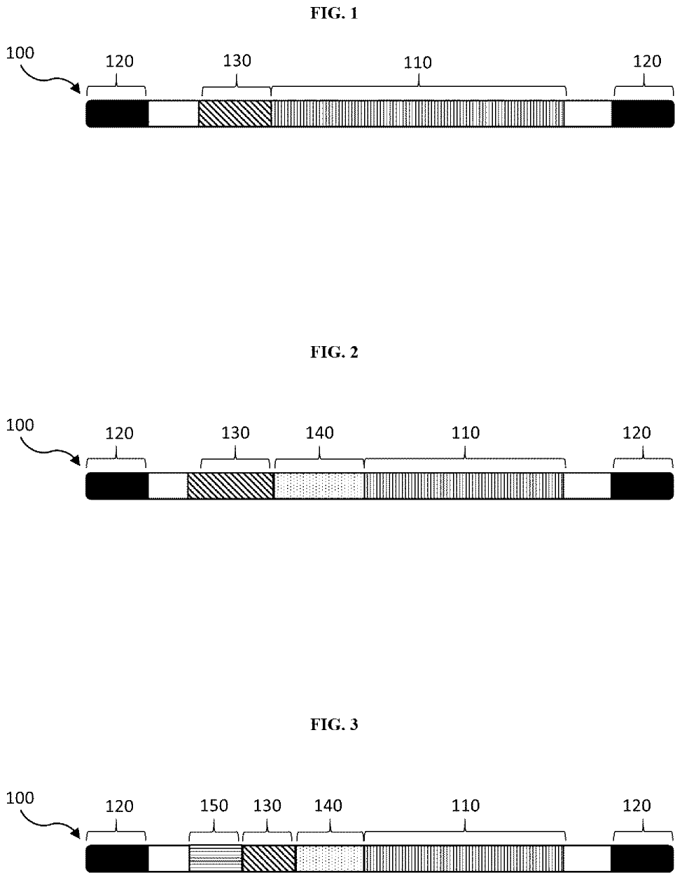

In one embodiment, the viral genome may comprise the components as shown in FIG. 1. The payload region 110 is located within the viral genome 100. At the 5′ and/or the 3′ end of the viral genome 100 there may be at least one inverted terminal repeat (ITR) 120. Between the 5′ ITR 120 and the payload region 110, there may be a promoter region 130. In one embodiment, the payload region may comprise at least one modulatory polynucleotide.

In one embodiment, the viral genome 100 may comprise the components as shown in FIG. 2. The payload region 110 is located within the viral genome 100. At the 5′ and/or the 3′ end of the viral genome 100 there may be at least one inverted terminal repeat (ITR) 120. Between the 5′ ITR 120 and the payload region 110, there may be a promoter region 130. Between the promoter region 130 and the payload region 110, there may be an intron region 140. In one embodiment, the payload region may comprise at least one modulatory polynucleotide.

In one embodiment, the viral genome 100 may comprise the components as shown in FIG. 3. At the 5′ and/or the 3′ end of the viral genome 100 there may be at least one inverted terminal repeat (ITR) 120. Within the viral genome 100, there may be an enhancer region 150, a promoter region 130, an intron region 140, and a payload region 110. In one embodiment, the payload region may comprise at least one modulatory polynucleotide.

In one embodiment, the viral genome 100 may comprise the components as shown in FIG. 4. At the 5′ and/or the 3′ end of the viral genome 100 there may be at least one inverted terminal repeat (ITR) 120. Within the viral genome 100, there may be an enhancer region 150, a promoter region 130, an intron region 140, a payload region 110, and a polyadenylation signal sequence region 160. In one embodiment, the payload region may comprise at least one modulatory polynucleotide.

In one embodiment, the viral genome 100 may comprise the components as shown in FIG. 5. At the 5′ and/or the 3′ end of the viral genome 100 there may be at least one inverted terminal repeat (ITR) 120. Within the viral genome 100, there may be at least one MCS region 170, an enhancer region 150, a promoter region 130, an intron region 140, a payload region 110, and a polyadenylation signal sequence region 160. In one embodiment, the payload region may comprise at least one modulatory polynucleotide.

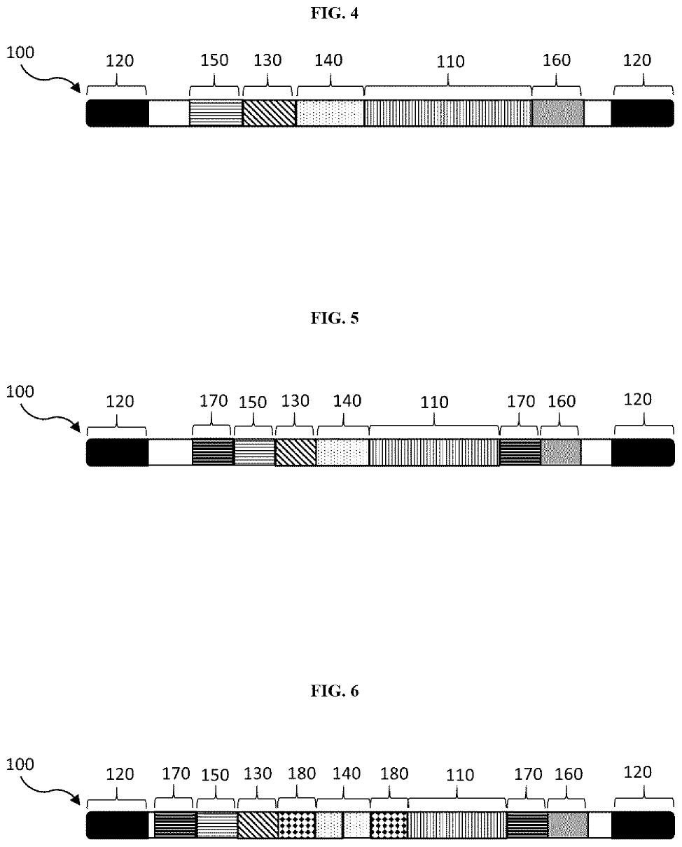

In one embodiment, the viral genome 100 may comprise the components as shown in FIG. 6. At the 5′ and/or the 3′ end of the viral genome 100 there may be at least one inverted terminal repeat (ITR) 120. Within the viral genome 100, there may be at least one MCS region 170, an enhancer region 150, a promoter region 130, at least one exon region 180, at least one intron region 140, a payload region 110, and a polyadenylation signal sequence region 160. In one embodiment, the payload region may comprise at least one modulatory polynucleotide.

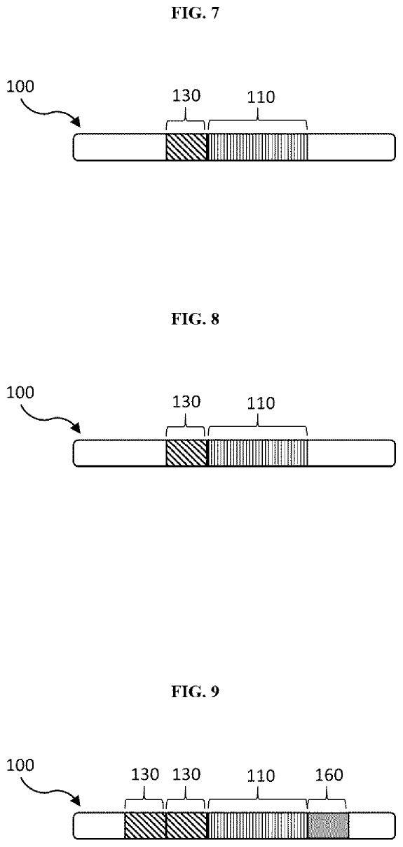

In one embodiment, the viral genome 100 may comprise the components as shown in FIGS. 7 and 8. Within the viral genome 100, there may be at least one promoter region 130, and a payload region 110. In one embodiment, the payload region may comprise at least one modulatory polynucleotide.

In one embodiment, the viral genome 100 may comprise the components as shown in FIG. 9. Within the viral genome 100, there may be at least one promoter region 130, a payload region 110, and a polyadenylation signal sequence region 160. In one embodiment, the payload region may comprise at least one modulatory polynucleotide.

### Viral Genome Size

In one embodiment, the viral genome which comprises a payload described herein, may be single stranded or double stranded viral genome. The size of the viral genome may be small, medium, large or the maximum size. Additionally, the viral genome may comprise a promoter and a polyA tail.

In one embodiment, the viral genome which comprises a payload described herein, may be a small single stranded viral genome. A small single stranded viral genome may be 2.7 to 3.5 kb in size such as about 2.7, 2.8, 2.9, 3.0, 3.1, 3.2, 3.3, 3.4, and 3.5 kb in size. As a non-limiting example, the small single stranded viral genome may be 3.2 kb in size. Additionally, the viral genome may comprise a promoter and a polyA tail.

In one embodiment, the viral genome which comprises a payload described herein, may be a small double stranded viral genome. A small double stranded viral genome may be 1.3 to 1.7 kb in size such as about 1.3, 1.4, 1.5, 1.6, and 1.7 kb in size. As a non-limiting example, the small double stranded viral genome may be 1.6 kb in size. Additionally, the viral genome may comprise a promoter and a polyA tail.

In one embodiment, the viral genome which comprises a payload described herein, may a medium single stranded viral genome. A medium single stranded viral genome may be 3.6 to 4.3 kb in size such as about 3.6, 3.7, 3.8, 3.9, 4.0, 4.1, 4.2 and 4.3 kb in size. As a non-limiting example, the medium single stranded viral genome may be 4.0 kb in size. Additionally, the viral genome may comprise a promoter and a polyA tail.

In one embodiment, the viral genome which comprises a payload described herein, may be a medium double stranded viral genome. A medium double stranded viral genome may be 1.8 to 2.1 kb in size such as about 1.8, 1.9, 2.0, and 2.1 kb in size. As a non-limiting example, the medium double stranded viral genome may be 2.0 kb in size. Additionally, the viral genome may comprise a promoter and a polyA tail.

In one embodiment, the viral genome which comprises a payload described herein, may be a large single stranded viral genome. A large single stranded viral genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded viral genome may be 4.7 kb in size. As another non-limiting example, the large single stranded viral genome may be 4.8 kb in size. As yet another non-limiting example, the large single stranded viral genome may be 6.0 kb in size. Additionally, the viral genome may comprise a promoter and a polyA tail.

In one embodiment, the viral genome which comprises a payload described herein, may be a large double stranded viral genome. A large double stranded viral genome may be 2.2 to 3.0 kb in size such as about 2.2, 2.3, 2.4, 2.5, 2.6, 2.7, 2.8, 2.9 and 3.0 kb in size. As a non-limiting example, the large double stranded viral genome may be 2.4 kb in size. Additionally, the viral genome may comprise a promoter and a polyA tail.

### Viral Genome Component: Inverted Terminal Repeats (ITRs)

The AAV particles of the present invention comprise a viral genome with at least one ITR region and a payload region. In one embodiment the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes of the invention may be comprised of naturally occurring polynucleotide sequences or recombinantly derived polynucleotide sequences.

The ITRs may be derived from the same serotype as the capsid, selected from any of the serotypes listed in Table 1, or a derivative thereof. The ITR may be of a different serotype from the capsid. In one embodiment the AAV particle has more than one ITR. In a non-limiting example, the AAV particle has a viral genome comprising two ITRs. In one embodiment the ITRs are of the same serotype as one another. In another embodiment the ITRs are of different serotypes. Non-limiting examples include zero, one or both of the ITRs having the same serotype as the capsid. In one embodiment both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In one embodiment the ITRs are 140-142 nucleotides in length. Non limiting examples of ITR length are 102, 140, 141, 142, 145 nucleotides in length, and those having at least 95% identity thereto.

In one embodiment, the AAV particle comprises a nucleic acid sequence encoding an siRNA molecule which may be located near the 5′ end of the flip ITR in an expression vector. In another embodiment, the AAV particle comprises a nucleic acid sequence encoding an siRNA molecule may be located near the 3′ end of the flip ITR in an expression vector. In yet another embodiment, the AAV particle comprises a nucleic acid sequence encoding an siRNA molecule may be located near the 5′ end of the flop ITR in an expression vector. In yet another embodiment, the AAV particle comprises a nucleic acid sequence encoding an siRNA molecule may be located near the 3′ end of the flop ITR in an expression vector. In one embodiment, the AAV particle comprises a nucleic acid sequence encoding an siRNA molecule may be located between the 5′ end of the flip ITR and the 3′ end of the flop ITR in an expression vector. In one embodiment, the AAV particle comprises a nucleic acid sequence encoding an siRNA molecule may be located between (e.g., half-way between the 5′ end of the flip ITR and 3′ end of the flop ITR or the 3′ end of the flop ITR and the 5′ end of the flip ITR), the 3′ end of the flip ITR and the 5′ end of the flip ITR in an expression vector. As a non-limiting example, the AAV particle comprises a nucleic acid sequence encoding an siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the 5′ or 3′ end of an ITR (e.g., Flip or Flop ITR) in an expression vector. As a non-limiting example, the AAV particle comprises a nucleic acid sequence encoding an siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides upstream from the 5′ or 3′ end of an ITR (e.g., Flip or Flop ITR) in an expression vector. As another non-limiting example, the AAV particle comprises a nucleic acid sequence encoding an siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the 5′ or 3′ end of an ITR (e.g., Flip or Flop ITR) in an expression vector. As another non-limiting example, the AAV particle comprises a nucleic acid sequence encoding an siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 upstream from the 5′ or 3′ end of an ITR (e.g., Flip or Flop ITR) in an expression vector. As a non-limiting example, the AAV particle comprises a nucleic acid sequence encoding an siRNA molecule may be located within the first 1%, 2%, 3%, 4%, 5%, 6%, 7%, 8%, 9%, 10%, 15%, 20%, 25% or more than 25% of the nucleotides upstream from the 5′ or 3′ end of an ITR (e.g., Flip or Flop ITR) in an expression vector. As another non-limiting example, the AAV particle comprises a nucleic acid sequence encoding an siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the 5′ or 3′ end of an ITR (e.g., Flip or Flop ITR) in an expression vector.

### Viral Genome Component: Promoters

In one embodiment, the payload region of the viral genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

A person skilled in the art may recognize that expression of the polypeptides of the invention in a target cell may require a specific promoter, including but not limited to, a promoter that is species specific, inducible, tissue-specific, or cell cycle-specific (Parr et al., Nat. Med. 3:1145-9 (1997); the contents of which are herein incorporated by reference in their entirety).

In one embodiment, the promoter is deemed to be efficient when it drives expression of the polypeptide(s) encoded in the payload region of the viral genome of the AAV particle.

In one embodiment, the promoter is a promoter deemed to be efficient to drive the expression of the modulatory polynucleotide.

In one embodiment, the promoter is a promoter deemed to be efficient when it drives expression in the cell being targeted.

In one embodiment, the promoter drives expression of the payload for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years.

In one embodiment, the promoter drives expression of the payload for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

Promoters may be naturally occurring or non-naturally occurring. Non-limiting examples of promoters include viral promoters, plant promoters and mammalian promoters. In some embodiments, the promoters may be human promoters. In some embodiments, the promoter may be truncated.

Promoters which drive or promote expression in most tissues include, but are not limited to, human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US 20110212529, the contents of which are herein incorporated by reference in their entirety)

Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In one embodiment, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800.

In one embodiment, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. In one embodiment, the promoter is a combination of a 382 nucleotide CMV-enhancer sequence and a 260 nucleotide CBA-promoter sequence.

In one embodiment, the viral genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3).

Yu et al. (Molecular Pain 2011, 7:63; the contents of which are herein incorporated by reference in their entirety) evaluated the expression of eGFP under the CAG, EFIα, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015; the contents of which are herein incorporated by reference in its entirety) evaluated the expression of eGFP in AAV8 with CMV and UBC promoters and AAV2 with the CMV promoter after injection in the motor cortex. Intranasal administration of a plasmid containing a UBC or EFIα promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009; the contents of which are herein incorporated by reference in its entirety) evaluated an HβH construct with a hGUSB promoter, a HSV-1LAT promoter and an NSE promoter and found that the HβH construct showed weaker expression than NSE in mouse brain. Passini and Wolfe (J. Virol. 2001, 12382-12392, the contents of which are herein incorporated by reference in its entirety) evaluated the long term effects of the HβH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in their entirety) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter which are both absent in the liver but NFH is abundant in the sensory proprioceptive neurons, brain and spinal cord and NFH is present in the heart. Scn8a is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. Identification of evolutionary conserved, functional noncoding elements in the promoter region of the sodium channel gene SCN8A, Mamm Genome (2007) 18:723-731; and Raymond et al. Expression of Alternatively Spliced Sodium Channel α-subunit genes, Journal of Biological Chemistry (2004) 279(44) 46234-46241; the contents of each of which are herein incorporated by reference in their entireties).

Any of promoters taught by the aforementioned Yu, Soderblom, Gill, Husain, Passini, Xu, Drews or Raymond may be used in the present inventions.

In one embodiment, the promoter is not cell specific.

In one embodiment, the promoter is an ubiquitin c (UBC) promoter. The UBC promoter may have a size of 300-350 nucleotides. As a non-limiting example, the UBC promoter is 332 nucleotides.

In one embodiment, the promoter is a β-glucuronidase (GUSB) promoter. The GUSB promoter may have a size of 350-400 nucleotides. As a non-limiting example, the GUSB promoter is 378 nucleotides.

In one embodiment, the promoter is a neurofilament light (NFL) promoter. The NFL promoter may have a size of 600-700 nucleotides. As a non-limiting example, the NFL promoter is 650 nucleotides. As a non-limiting example, the construct may be AAV-promoter-CMV/globin intron-modulatory polynucleotide-RBG, where the AAV may be self-complementary and the AAV may be the DJ serotype.

In one embodiment, the promoter is a neurofilament heavy (NFH) promoter. The NFH promoter may have a size of 900-950 nucleotides. As a non-limiting example, the NFH promoter is 920 nucleotides. As a non-limiting example, the construct may be AAV-promoter-CMV/globin intron-modulatory polynucleotide-RBG, where the AAV may be self-complementary and the AAV may be the DJ serotype.

In one embodiment, the promoter is a scn8a promoter. The scn8a promoter may have a size of 450-500 nucleotides. As a non-limiting example, the scn8a promoter is 470 nucleotides. As a non-limiting example, the construct may be AAV-promoter-CMV/globin intron-modulatory polynucleotide-RBG, where the AAV may be self-complementary and the AAV may be the DJ serotype

In one embodiment, the viral genome comprises a Pol III promoter.

In one embodiment, the viral genome comprises a P1 promoter.

In one embodiment, the viral genome comprises a FXN promoter.

In one embodiment, the promoter is a phosphoglycerate kinase 1 (PGK) promoter.

In one embodiment, the promoter is a chicken β-actin (CBA) promoter.

In one embodiment, the promoter is a CAG promoter which is a construct comprising the cytomegalovirus (CMV) enhancer fused to the chicken beta-actin (CBA) promoter.

In one embodiment, the promoter is a cytomegalovirus (CMV) promoter.

In one embodiment, the viral genome comprises a Pol III promoter, for example, a Pol III type 3 promoter.

In one embodiment, comprises an U3, U6, U7, 7SK, H1, or MRP, EBER, seleno-cysteine tRNA, 7SL, adenovirus VA-1, or telomerase gene promoter.

In one embodiment, the viral genome comprises an H1 promoter.

In one embodiment, the viral genome comprises a U6 promoter.

In one embodiment, the promoter is a liver or a skeletal muscle promoter. Non-limiting examples of liver promoters include human α-1-antitrypsin (hAAT) and thyroxine binding globulin (TBG). Non-limiting examples of skeletal muscle promoters include Desmin, MCK or synthetic C5-12.

In one embodiment, the promoter is a RNA pol III promoter. As a non-limiting example, the RNA pol III promoter is U6. As a non-limiting example, the RNA pol III promoter is H1.

In one embodiment, the promoter is a RNA Pol II promoter, including, for example, a truncated RNA Pol II promoter.

In one embodiment, the viral genome comprises two promoters. As a non-limiting example, the promoters are an EF1α promoter and a CMV promoter.

In one embodiment, the viral genome comprises an enhancer element, a promoter and/or a 5′UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter, (9) GFAP promoter, (10) H1 promoter; and (11) U6 promoter.

In one embodiment, the viral genome comprises an engineered promoter.

In another embodiment the viral genome comprises a promoter from a naturally expressed protein.

### Viral Genome Component: Untranslated Regions (UTRs)

By definition, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the viral genomes of the AAV particles of the invention to enhance expression in hepatic cell lines or liver.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In one embodiment, the 5′UTR in the viral genome includes a Kozak sequence.

In one embodiment, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In one embodiment, the 3′ UTR of the viral genome may include an oligo(dT) sequence for templated addition of a poly-A tail.

In one embodiment, the viral genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA, which sequence has perfect Watson-Crick complementarity to the miRNA target sequence of the nucleic acid.

In one embodiment, the viral genome may be engineered to include, alter or remove at least one miRNA binding site, sequence or seed region.

Any UTR from any gene known in the art may be incorporated into the viral genome of the AAV particle. These UTRs, or portions thereof, may be placed in the same orientation as in the gene from which they were selected or they may be altered in orientation or location. In one embodiment, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In one embodiment, the viral genome of the AAV particle comprises at least one artificial UTRs which is not a variant of a wild type UTR.

In one embodiment, the viral genome of the AAV particle comprises UTRs which have been selected from a family of transcripts whose proteins share a common function, structure, feature or property.

### Viral Genome Component: Polyadenylation Sequence

In one embodiment, the viral genome of the AAV particles of the present invention comprise at least one polyadenylation sequence. The viral genome of the AAV particle may comprise a polyadenylation sequence between the 3′ end of the payload coding sequence and the 5′ end of the 3′ITR.

In one embodiment, the polyadenylation sequence or “polyA sequence” may range from absent to about 500 nucleotides in length. The polyadenylation sequence may be, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and 500 nucleotides in length.

In one embodiment, the polyadenylation sequence is 50-100 nucleotides in length.

In one embodiment, the polyadenylation sequence is 50-150 nucleotides in length.

In one embodiment, the polyadenylation sequence is 50-160 nucleotides in length.

In one embodiment, the polyadenylation sequence is 50-200 nucleotides in length.

In one embodiment, the polyadenylation sequence is 60-100 nucleotides in length.

In one embodiment, the polyadenylation sequence is 60-150 nucleotides in length.

In one embodiment, the polyadenylation sequence is 60-160 nucleotides in length.

In one embodiment, the polyadenylation sequence is 60-200 nucleotides in length.

In one embodiment, the polyadenylation sequence is 70-100 nucleotides in length.

In one embodiment, the polyadenylation sequence is 70-150 nucleotides in length.

In one embodiment, the polyadenylation sequence is 70-160 nucleotides in length.

In one embodiment, the polyadenylation sequence is 70-200 nucleotides in length.

In one embodiment, the polyadenylation sequence is 80-100 nucleotides in length.

In one embodiment, the polyadenylation sequence is 80-150 nucleotides in length.

In one embodiment, the polyadenylation sequence is 80-160 nucleotides in length.

In one embodiment, the polyadenylation sequence is 80-200 nucleotides in length.

In one embodiment, the polyadenylation sequence is 90-100 nucleotides in length.

In one embodiment, the polyadenylation sequence is 90-150 nucleotides in length.

In one embodiment, the polyadenylation sequence is 90-160 nucleotides in length.

In one embodiment, the polyadenylation sequence is 90-200 nucleotides in length.

In one embodiment, the AAV particle comprises a nucleic acid sequence encoding an siRNA molecule may be located upstream of the polyadenylation sequence in an expression vector. Further, the AAV particle comprises a nucleic acid sequence encoding an siRNA molecule may be located downstream of a promoter such as, but not limited to, CMV, U6, CAG, CBA or a CBA promoter with a SV40 intron or a human betaglobin intron in an expression vector. As a non-limiting example, the AAV particle comprises a nucleic acid sequence encoding an siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the AAV particle comprises a nucleic acid sequence encoding an siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the AAV particle comprises a nucleic acid sequence encoding an siRNA molecule may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence in an expression vector. As another non-limiting example, the AAV particle comprises a nucleic acid sequence encoding an siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

In one embodiment, the AAV particle comprises a rabbit globin polyadenylation (polyA) signal sequence.

In one embodiment, the AAV particle comprises a human growth hormone polyadenylation (polyA) signal sequence.

### Viral Genome Component: Introns

In one embodiment, the payload region comprises at least one element to enhance the expression such as one or more introns or portions thereof. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In one embodiment, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500.

In one embodiment, the AAV viral genome may comprise a promoter such as, but not limited to, CMV or U6. As a non-limiting example, the promoter for the AAV comprising the nucleic acid sequence for the siRNA molecules of the present invention is a CMV promoter. As another non-limiting example, the promoter for the AAV comprising the nucleic acid sequence for the siRNA molecules of the present invention is a U6 promoter.

In one embodiment, the AAV viral genome may comprise a CMV promoter.

In one embodiment, the AAV viral genome may comprise a U6 promoter.

In one embodiment, the AAV viral genome may comprise a CMV and a U6 promoter.

In one embodiment, the AAV viral genome may comprise a Pol III promoter.

In one embodiment, the AAV viral genome may comprise a Pol III type 3 promoter.

In one embodiment, the AAV viral genome may comprise a H1 promoter.

In one embodiment, the AAV viral genome may comprise a U6 promoter.

In one embodiment, the AAV viral genome may comprise a CBA promoter.

In one embodiment, the encoded siRNA molecule may be located downstream of a promoter in an expression vector such as, but not limited to, CMV, U6, H1, CBA, CAG, or a CBA promoter with an intron such as SV40 or others known in the art. Further, the encoded siRNA molecule may also be located upstream of the polyadenylation sequence in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the encoded siRNA molecule may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence in an expression vector. As another non-limiting example, the encoded siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

### Viral Genome Component: Filler Sequence

In one embodiment, the viral genome comprises one or more filler sequences.

In one embodiment, the viral genome comprises one or more filler sequences in order to have the length of the viral genome be the optimal size for packaging. As a non-limiting example, the viral genome comprises at least one filler sequence in order to have the length of the viral genome be about 2.3 kb. As a non-limiting example, the viral genome comprises at least one filler sequence in order to have the length of the viral genome be about 4.6 kb.

In one embodiment, the viral genome comprises one or more filler sequences in order to reduce the likelihood that a hairpin structure of the vector genome (e.g., a modulatory polynucleotide described herein) may be read as an inverted terminal repeat (ITR) during expression and/or packaging. As a non-limiting example, the viral genome comprises at least one filler sequence in order to have the length of the viral genome be about 2.3 kb. As a non-limiting example, the viral genome comprises at least one filler sequence in order to have the length of the viral genome be about 4.6 kb

In one embodiment, the viral genome is a single stranded (ss) viral genome and comprises one or more filler sequences which have a length about between 0.1 kb-3.8 kb, such as, but not limited to, 0.1 kb, 0.2 kb, 0.3 kb, 0.4 kb, 0.5 kb, 0.6 kb, 0.7 kb, 0.8 kb, 0.9 kb, 1 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, or 3.8 kb. As a non-limiting example, the total length filler sequence in the vector genome is 3.1 kb. As a non-limiting example, the total length filler sequence in the vector genome is 2.7 kb. As a non-limiting example, the total length filler sequence in the vector genome is 0.8 kb. As a non-limiting example, the total length filler sequence in the vector genome is 0.4 kb. As a non-limiting example, the length of each filler sequence in the vector genome is 0.8 kb. As a non-limiting example, the length of each filler sequence in the vector genome is 0.4 kb.

In one embodiment, the viral genome is a self-complementary (sc) viral genome and comprises one or more filler sequences which have a length about between 0.1 kb-1.5 kb, such as, but not limited to, 0.1 kb, 0.2 kb, 0.3 kb, 0.4 kb, 0.5 kb, 0.6 kb, 0.7 kb, 0.8 kb, 0.9 kb, 1 kb, 1.1 kb, 1.2 kb, 1.3 kb, 1.4 kb, or 1.5 kb. As a non-limiting example, the total length filler sequence in the vector genome is 0.8 kb. As a non-limiting example, the total length filler sequence in the vector genome is 0.4 kb. As a non-limiting example, the length of each filler sequence in the vector genome is 0.8 kb. As a non-limiting example, the length of each filler sequence in the vector genome is 0.4 kb

In one embodiment, the viral genome comprises any portion of a filler sequence. The viral genome may comprise 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99% of a filler sequence.

In one embodiment, the viral genome is a single stranded (ss) viral genome and comprises one or more filler sequences in order to have the length of the viral genome be about 4.6 kb. As a non-limiting example, the viral genome comprises at least one filler sequence and the filler sequence is located 3′ to the 5′ ITR sequence. As a non-limiting example, the viral genome comprises at least one filler sequence and the filler sequence is located 5′ to a promoter sequence. As a non-limiting example, the viral genome comprises at least one filler sequence and the filler sequence is located 3′ to the polyadenylation signal sequence. As a non-limiting example, the viral genome comprises at least one filler sequence and the filler sequence is located 5′ to the 3′ ITR sequence. As a non-limiting example, the viral genome comprises at least one filler sequence, and the filler sequence is located between two intron sequences. As a non-limiting example, the viral genome comprises at least one filler sequence, and the filler sequence is located within an intron sequence. As a non-limiting example, the viral genome comprises two filler sequences, and the first filler sequence is located 3′ to the 5′ ITR sequence and the second filler sequence is located 3′ to the polyadenylation signal sequence. As a non-limiting example, the viral genome comprises two filler sequences, and the first filler sequence is located 5′ to a promoter sequence and the second filler sequence is located 3′ to the polyadenylation signal sequence. As a non-limiting example, the viral genome comprises two filler sequences, and the first filler sequence is located 3′ to the 5′ ITR sequence and the second filler sequence is located 5′ to the 5′ ITR sequence.

In one embodiment, the viral genome is a self-complementary (sc) viral genome and comprises one or more filler sequences in order to have the length of the viral genome be about 2.3 kb. As a non-limiting example, the viral genome comprises at least one filler sequence and the filler sequence is located 3′ to the 5′ ITR sequence. As a non-limiting example, the viral genome comprises at least one filler sequence and the filler sequence is located 5′ to a promoter sequence. As a non-limiting example, the viral genome comprises at least one filler sequence and the filler sequence is located 3′ to the polyadenylation signal sequence. As a non-limiting example, the viral genome comprises at least one filler sequence and the filler sequence is located 5′ to the 3′ ITR sequence. As a non-limiting example, the viral genome comprises at least one filler sequence, and the filler sequence is located between two intron sequences. As a non-limiting example, the viral genome comprises at least one filler sequence, and the filler sequence is located within an intron sequence. As a non-limiting example, the viral genome comprises two filler sequences, and the first filler sequence is located 3′ to the 5′ ITR sequence and the second filler sequence is located 3′ to the polyadenylation signal sequence. As a non-limiting example, the viral genome comprises two filler sequences, and the first filler sequence is located 5′ to a promoter sequence and the second filler sequence is located 3′ to the polyadenylation signal sequence. As a non-limiting example, the viral genome comprises two filler sequences, and the first filler sequence is located 3′ to the 5′ ITR sequence and the second filler sequence is located 5′ to the 5′ ITR sequence.

In one embodiment, the viral genome may comprise one or more filler sequences between one of more regions of the viral genome. In one embodiment, the filler region may be located before a region such as, but not limited to, a payload region, an inverted terminal repeat (ITR), a promoter region, an intron region, an enhancer region, a polyadenylation signal sequence region, a multiple cloning site (MCS) region, and/or an exon region. In one embodiment, the filler region may be located after a region such as, but not limited to, a payload region, an inverted terminal repeat (ITR), a promoter region, an intron region, an enhancer region, a polyadenylation signal sequence region, a multiple cloning site (MCS) region, and/or an exon region. In one embodiment, the filler region may be located before and after a region such as, but not limited to, a payload region, an inverted terminal repeat (ITR), a promoter region, an intron region, an enhancer region, a polyadenylation signal sequence region, a multiple cloning site (MCS) region, and/or an exon region.

In one embodiment, the viral genome may comprise one or more filler sequences which bifurcates at least one region of the viral genome. The bifurcated region of the viral genome may comprise 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99% of the of the region to the 5′ of the filler sequence region. As a non-limiting example, the filler sequence may bifurcate at least one region so that 10% of the region is located 5′ to the filler sequence and 90% of the region is located 3′ to the filler sequence. As a non-limiting example, the filler sequence may bifurcate at least one region so that 20% of the region is located 5′ to the filler sequence and 80% of the region is located 3′ to the filler sequence. As a non-limiting example, the filler sequence may bifurcate at least one region so that 30% of the region is located 5′ to the filler sequence and 70% of the region is located 3′ to the filler sequence. As a non-limiting example, the filler sequence may bifurcate at least one region so that 40% of the region is located 5′ to the filler sequence and 60% of the region is located 3′ to the filler sequence. As a non-limiting example, the filler sequence may bifurcate at least one region so that 50% of the region is located 5′ to the filler sequence and 50% of the region is located 3′ to the filler sequence. As a non-limiting example, the filler sequence may bifurcate at least one region so that 60% of the region is located 5′ to the filler sequence and 40% of the region is located 3′ to the filler sequence. As a non-limiting example, the filler sequence may bifurcate at least one region so that 70% of the region is located 5′ to the filler sequence and 30% of the region is located 3′ to the filler sequence. As a non-limiting example, the filler sequence may bifurcate at least one region so that 80% of the region is located 5′ to the filler sequence and 20% of the region is located 3′ to the filler sequence. As a non-limiting example, the filler sequence may bifurcate at least one region so that 90% of the region is located 5′ to the filler sequence and 10% of the region is located 3′ to the filler sequence.

In one embodiment, the viral genome comprises a filler sequence after the 5′ ITR.

In one embodiment, the viral genome comprises a filler sequence after the promoter region. In one embodiment, the viral genome comprises a filler sequence after the payload region. In one embodiment, the viral genome comprises a filler sequence after the intron region. In one embodiment, the viral genome comprises a filler sequence after the enhancer region. In one embodiment, the viral genome comprises a filler sequence after the polyadenylation signal sequence region. In one embodiment, the viral genome comprises a filler sequence after the MCS region. In one embodiment, the viral genome comprises a filler sequence after the exon region.

In one embodiment, the viral genome comprises a filler sequence before the promoter region. In one embodiment, the viral genome comprises a filler sequence before the payload region. In one embodiment, the viral genome comprises a filler sequence before the intron region. In one embodiment, the viral genome comprises a filler sequence before the enhancer region. In one embodiment, the viral genome comprises a filler sequence before the polyadenylation signal sequence region. In one embodiment, the viral genome comprises a filler sequence before the MCS region. In one embodiment, the viral genome comprises a filler sequence before the exon region.

In one embodiment, the viral genome comprises a filler sequence before the 3′ ITR.

In one embodiment, a filler sequence may be located between two regions, such as, but not limited to, the 5′ ITR and the promoter region. In one embodiment, a filler sequence may be located between two regions, such as, but not limited to, the 5′ ITR and the payload region. In one embodiment, a filler sequence may be located between two regions, such as, but not limited to, the 5′ ITR and the intron region. In one embodiment, a filler sequence may be located between two regions, such as, but not limited to, the 5′ ITR and the enhancer region. In one embodiment, a filler sequence may be located between two regions, such as, but not limited to, the 5′ ITR and the polyadenylation signal sequence region. In one embodiment, a filler sequence may be located between two regions, such as, but not limited to, the 5′ ITR and the MCS region.

In one embodiment, a filler sequence may be located between two regions, such as, but not limited to, the 5′ ITR and the exon region.

In one embodiment, a filler sequence may be located between two regions, such as, but not limited to, the promoter region and the payload region. In one embodiment, a filler sequence may be located between two regions, such as, but not limited to, the promoter region and the intron region. In one embodiment, a filler sequence may be located between two regions, such as, but not limited to, the promoter region and the enhancer region. In one embodiment, a filler sequence may be located between two regions, such as, but not limited to, the promoter region and the polyadenylation signal sequence region. In one embodiment, a filler sequence may be located between two regions, such as, but not limited to, the promoter region and the MCS region. In one embodiment, a filler sequence may be located between two regions, such as, but not limited to, the promoter region and the exon region. In one embodiment, a filler sequence may be located between two regions, such as, but not limited to, the promoter region and the 3′ ITR.

In one embodiment, a filler sequence may be located between two regions, such as, but not limited to, the payload region and the intron region. In one embodiment, a filler sequence may be located between two regions, such as, but not limited to, the payload region and the enhancer region. In one embodiment, a filler sequence may be located between two regions, such as, but not limited to, the payload region and the polyadenylation signal sequence region. In one embodiment, a filler sequence may be located between two regions, such as, but not limited to, the payload region and the MCS region. In one embodiment, a filler sequence may be located between two regions, such as, but not limited to, the payload region and the exon region.

In one embodiment, a filler sequence may be located between two regions, such as, but not limited to, the payload region and the 3′ ITR.

In one embodiment, a filler sequence may be located between two regions, such as, but not limited to, the intron region and the enhancer region. In one embodiment, a filler sequence may be located between two regions, such as, but not limited to, the intron region and the polyadenylation signal sequence region. In one embodiment, a filler sequence may be located between two regions, such as, but not limited to, the intron region and the MCS region. In one embodiment, a filler sequence may be located between two regions, such as, but not limited to, the intron region and the exon region. In one embodiment, a filler sequence may be located between two regions, such as, but not limited to, the intron region and the 3′ ITR. In one embodiment, a filler sequence may be located between two regions, such as, but not limited to, the enhancer region and the polyadenylation signal sequence region. In one embodiment, a filler sequence may be located between two regions, such as, but not limited to, the enhancer region and the MCS region. In one embodiment, a filler sequence may be located between two regions, such as, but not limited to, the enhancer region and the exon region. In one embodiment, a filler sequence may be located between two regions, such as, but not limited to, the enhancer region and the 3′ ITR.

In one embodiment, a filler sequence may be located between two regions, such as, but not limited to, the polyadenylation signal sequence region and the MCS region. In one embodiment, a filler sequence may be located between two regions, such as, but not limited to, the polyadenylation signal sequence region and the exon region. In one embodiment, a filler sequence may be located between two regions, such as, but not limited to, the polyadenylation signal sequence region and the 3′ ITR.

In one embodiment, a filler sequence may be located between two regions, such as, but not limited to, the MCS region and the exon region. In one embodiment, a filler sequence may be located between two regions, such as, but not limited to, the MCS region and the 3′ ITR.

In one embodiment, a filler sequence may be located between two regions, such as, but not limited to, the exon region and the 3′ ITR.

In one embodiment, a viral genome may comprise two filler sequences, the first filler sequence may be located between the 5′ ITR and promoter region, and the second filler sequence may be located between the promoter region and payload region. In one embodiment, a viral genome may comprise two filler sequences, the first filler sequence may be located between the 5′ ITR and promoter region, and the second filler sequence may be located between the promoter region and intron region. In one embodiment, a viral genome may comprise two filler sequences, the first filler sequence may be located between the 5′ ITR and promoter region, and the second filler sequence may be located between the promoter region and enhancer region. In one embodiment, a viral genome may comprise two filler sequences, the first filler sequence may be located between the 5′ ITR and promoter region, and the second filler sequence may be located between the promoter region and polyadenylation signal sequence region. In one embodiment, a viral genome may comprise two filler sequences, the first filler sequence may be located between the 5′ ITR and promoter region, and the second filler sequence may be located between the promoter region and MCS region. In one embodiment, a viral genome may comprise two filler sequences, the first filler sequence may be located between the 5′ ITR and promoter region, and the second filler sequence may be located between the promoter region and exon region. In one embodiment, a viral genome may comprise two filler sequences, the first filler sequence may be located between the 5′ ITR and promoter region, and the second filler sequence may be located between the promoter region and 3′ ITR. In one embodiment, a viral genome may comprise two filler sequences, the first filler sequence may be located between the 5′ ITR and promoter region, and the second filler sequence may be located between the payload region and intron region. In one embodiment, a viral genome may comprise two filler sequences, the first filler sequence may be located between the 5′ ITR and promoter region, and the second filler sequence may be located between the payload region and enhancer region. In one embodiment, a viral genome may comprise two filler sequences, the first filler sequence may be located between the 5′ ITR and promoter region, and the second filler sequence may be located between the payload region and polyadenylation signal sequence region. In one embodiment, a viral genome may comprise two filler sequences, the first filler sequence may be located between the 5′ ITR and promoter region, and the second filler sequence may be located between the payload region and MCS region. In one embodiment, a viral genome may comprise two filler sequences, the first filler sequence may be located between the 5′ ITR and promoter region, and the second filler sequence may be located between the payload region and exon region. In one embodiment, a viral genome may comprise two filler sequences, the first filler sequence may be located between the 5′ ITR and promoter region, and the second filler sequence may be located between the payload region and 3′ ITR. In one embodiment, a viral genome may comprise two filler sequences, the first filler sequence may be located between the 5′ ITR and promoter region, and the second filler sequence may be located between the intron region and enhancer region. In one embodiment, a viral genome may comprise two filler sequences, the first filler sequence may be located between the 5′ ITR and promoter region, and the second filler sequence may be located between the intron region and polyadenylation signal sequence region. In one embodiment, a viral genome may comprise two filler sequences, the first filler sequence may be located between the 5′ ITR and promoter region, and the second filler sequence may be located between the intron region and MCS region. In one embodiment, a viral genome may comprise two filler sequences, the first filler sequence may be located between the 5′ ITR and promoter region, and the second filler sequence may be located between the intron region and exon region. In one embodiment, a viral genome may comprise two filler sequences, the first filler sequence may be located between the 5′ ITR and promoter region, and the second filler sequence may be located between the intron region and 3′ ITR. In one embodiment, a viral genome may comprise two filler sequences, the first filler sequence may be located between the 5′ ITR and promoter region, and the second filler sequence may be located between the enhancer region and polyadenylation signal sequence region. In one embodiment, a viral genome may comprise two filler sequences, the first filler sequence may be located between the 5′ ITR and promoter region, and the second filler sequence may be located between the enhancer region and MCS region. In one embodiment, a viral genome may comprise two filler sequences, the first filler sequence may be located between the 5′ ITR and promoter region, and the second filler sequence may be located between the enhancer region and exon region. In one embodiment, a viral genome may comprise two filler sequences, the first filler sequence may be located between the 5′ ITR and promoter region, and the second filler sequence may be located between the enhancer region and 3′ ITR. In one embodiment, a viral genome may comprise two filler sequences, the first filler sequence may be located between the 5′ ITR and promoter region, and the second filler sequence may be located between the polyadenylation signal sequence region and MCS region. In one embodiment, a viral genome may comprise two filler sequences, the first filler sequence may be located between the 5′ ITR and promoter region, and the second filler sequence may be located between the polyadenylation signal sequence region and exon region. In one embodiment, a viral genome may comprise two filler sequences, the first filler sequence may be located between the 5′ ITR and promoter region, and the second filler sequence may be located between the polyadenylation signal sequence region and 3′ ITR. In one embodiment, a viral genome may comprise two filler sequences, the first filler sequence may be located between the 5′ ITR and promoter region, and the second filler sequence may be located between the MCS region and exon region. In one embodiment, a viral genome may comprise two filler sequences, the first filler sequence may be located between the 5′ ITR and promoter region, and the second filler sequence may be located between the MCS region and 3′ ITR. In one embodiment, a viral genome may comprise two filler sequences, the first filler sequence may be located between the 5′ ITR and promoter region, and the second filler sequence may be located between the exon region and 3′ ITR.

In one embodiment, a viral genome may comprise two filler sequences, the first filler sequence may be located between the 5′ ITR and payload region, and the second filler sequence may be located between the promoter region and payload region. In one embodiment, a viral genome may comprise two filler sequences, the first filler sequence may be located between the 5′ ITR and payload region, and the second filler sequence may be located between the promoter region and intron region. In one embodiment, a viral genome may comprise two filler sequences, the first filler sequence may be located between the 5′ ITR and payload region, and the second filler sequence may be located between the promoter region and enhancer region. In one embodiment, a viral genome may comprise two filler sequences, the first filler sequence may be located between the 5′ ITR and payload region, and the second filler sequence may be located between the promoter region and polyadenylation signal sequence region. In one embodiment, a viral genome may comprise two filler sequences, the first filler sequence may be located between the 5′ ITR and payload region, and the second filler sequence may be located between the promoter region and MCS region. In one embodiment, a viral genome may comprise two filler sequences, the first filler sequence may be located between the 5′ ITR and payload region, and the second filler sequence may be located between the promoter region and exon region. In one embodiment, a viral genome may comprise two filler sequences, the first filler sequence may be located between the 5′ ITR and payload region, and the second filler sequence may be located between the promoter region and 3′ ITR. In one embodiment, a viral genome may comprise two filler sequences, the first filler sequence may be located between the 5′ ITR and payload region, and the second filler sequence may be located between the payload region and intron region. In one embodiment, a viral genome may comprise two filler sequences, the first filler sequence may be located between the 5′ ITR and payload region, and the second filler sequence may be located between the payload region and enhancer region. In one embodiment, a viral genome may comprise two filler sequences, the first filler sequence may be located between the 5′ ITR and payload region, and the second filler sequence may be located between the payload region and polyadenylation signal sequence region. In one embodiment, a viral genome may comprise two filler sequences, the first filler sequence may be located between the 5′ ITR and payload region, and the second filler sequence may be located between the payload region and MCS region. In one embodiment, a viral genome may comprise two filler sequences, the first filler sequence may be located between the 5′ ITR and payload region, and the second filler sequence may be located between the payload region and exon region. In one embodiment, a viral genome may comprise two filler sequences, the first filler sequence may be located between the 5′ ITR and payload region, and the second filler sequence may be located between the payload region and 3′ ITR. In one embodiment, a viral genome may comprise two filler sequences, the first filler sequence may be located between the 5′ ITR and payload region, and the second filler sequence may be located between the intron region and enhancer region. In one embodiment, a viral genome may comprise two filler sequences, the first filler sequence may be located between the 5′ ITR and payload region, and the second filler sequence may be located between the intron region and polyadenylation signal sequence region. In one embodiment, a viral genome may comprise two filler sequences, the first filler sequence may be located between the 5′ ITR and payload region, and the second filler sequence may be located between the intron region and MCS region. In one embodiment, a viral genome may comprise two filler sequences, the first filler sequence may be located between the 5′ ITR and payload region, and the second filler sequence may be located between the intron region and exon region. In one embodiment, a viral genome may comprise two filler sequences, the first filler sequence may be located between the 5′ ITR and payload region, and the second filler sequence may be located between the intron region and 3′ ITR. In one embodiment, a viral genome may comprise two filler sequences, the first filler sequence may be located between the 5′ ITR and payload region, and the second filler sequence may be located between the enhancer region and polyadenylation signal sequence region. In one embodiment, a viral genome may comprise two filler sequences, the first filler sequence may be located between the 5′ ITR and payload region, and the second filler sequence may be located between the enhancer region and MCS region. In one embodiment, a viral genome may comprise two filler sequences, the first filler sequence may be located between the 5′ ITR and payload region, and the second filler sequence may be located between the enhancer region and exon region. In one embodiment, a viral genome may comprise two filler sequences, the first filler sequence may be located between the 5′ ITR and payload region, and the second filler sequence may be located between the enhancer region and 3′ ITR. In one embodiment, a viral genome may comprise two filler sequences, the first filler sequence may be located between the 5′ ITR and payload region, and the second filler sequence may be located between the polyadenylation signal sequence region and MCS region. In one embodiment, a viral genome may comprise two filler sequences, the first filler sequence may be located between the 5′ ITR and payload region, and the second filler sequence may be located between the polyadenylation signal sequence region and exon region. In one embodiment, a viral genome may comprise two filler sequences, the first filler sequence may be located between the 5′ ITR and payload region, and the second filler sequence may be located between the polyadenylation signal sequence region and 3′ ITR. In one embodiment, a viral genome may comprise two filler sequences, the first filler sequence may be located between the 5′ ITR and payload region, and the second filler sequence may be located between the MCS region and exon region. In one embodiment, a viral genome may comprise two filler sequences, the first filler sequence may be located between the 5′ ITR and payload region, and the second filler sequence may be located between the MCS region and 3′ ITR. In one embodiment, a viral genome may comprise two filler sequences, the first filler sequence may be located between the 5′ ITR and payload region, and the second filler sequence may be located between the exon region and 3′ ITR.

In one embodiment, a viral genome may comprise two filler sequences, the first filler sequence may be located between the 5′ ITR and intron region, and the second filler sequence may be located between the promoter region and payload region. In one embodiment, a viral genome may comprise two filler sequences, the first filler sequence may be located between the 5′ ITR and intron region, and the second filler sequence may be located between the promoter region and intron region. In one embodiment, a viral genome may comprise two filler sequences, the first filler sequence may be located between the 5′ ITR and intron region, and the second filler sequence may be located between the promoter region and enhancer region. In one embodiment, a viral genome may comprise two filler sequences, the first filler sequence may be located between the 5′ ITR and intron region, and the second filler sequence may be located between the promoter region and polyadenylation signal sequence region. In one embodiment, a viral genome may comprise two filler sequences, the first filler sequence may be located between the 5′ ITR and intron region, and the second filler sequence may be located between the promoter region and MCS region. In one embodiment, a viral genome may comprise two filler sequences, the first filler sequence may be located between the 5′ ITR and intron region, and the second filler sequence may be located between the promoter region and exon region. In one embodiment, a viral genome may comprise two filler sequences, the first filler sequence may be located between the 5′ ITR and intron region, and the second filler sequence may be located between the promoter region and 3′ ITR. In one embodiment, a viral genome may comprise two filler sequences, the first filler sequence may be located between the 5′ ITR and intron region, and the second filler sequence may be located between the payload region and intron region. In one embodiment, a viral genome may comprise two filler sequences, the first filler sequence may be located between the 5′ ITR and intron region, and the second filler sequence may be located between the payload region and enhancer region. In one embodiment, a viral genome may comprise two filler sequences, the first filler sequence may be located between the 5′ ITR and intron region, and the second filler sequence may be located between the payload region and polyadenylation signal sequence region. In one embodiment, a viral genome may comprise two filler sequences, the first filler sequence may be located between the 5′ ITR and intron region, and the second filler sequence may be located between the payload region and MCS region. In one embodiment, a viral genome may comprise two filler sequences, the first filler sequence may be located between the 5′ ITR and intron region, and the second filler sequence may be located between the payload region and exon region. In one embodiment, a viral genome may comprise two filler sequences, the first filler sequence may be located between the 5′ ITR and intron region, and the second filler sequence may be located between the payload region and 3′ ITR. In one embodiment, a viral genome may comprise two filler sequences, the first filler sequence may be located between the 5′ ITR and intron region, and the second filler sequence may be located between the intron region and enhancer region. In one embodiment, a viral genome may comprise two filler sequences, the first filler sequence may be located between the 5′ ITR and intron region, and the second filler sequence may be located between the intron region and polyadenylation signal sequence region. In one embodiment, a viral genome may comprise two filler sequences, the first filler sequence may be located between the 5′ ITR and intron region, and the second filler sequence may be located between the intron region and MCS region. In one embodiment, a viral genome may comprise two filler sequences, the first filler sequence may be located between the 5′ ITR and intron region, and the second filler sequence may be located between the intron region and exon region. In one embodiment, a viral genome may comprise two filler sequences, the first filler sequence may be located between the 5′ ITR and intron region, and the second filler sequence may be located between the intron region and 3′ ITR. In one embodiment, a viral genome may comprise two filler sequences, the first filler sequence may be located between the 5′ ITR and intron region, and the second filler sequence may be located between the enhancer region and polyadenylation signal sequence region. In one embodiment, a viral genome may comprise two filler sequences, the first filler sequence may be located between the 5′ ITR and intron region, and the second filler sequence may be located between the enhancer region and MCS region. In one embodiment, a viral genome may comprise two filler sequences, the first filler sequence may be located between the 5′ ITR and intron region, and the second filler sequence may be located between the enhancer region and exon region. In one embodiment, a viral genome may comprise two filler sequences, the first filler sequence may be located between the 5′ ITR and intron region, and the second filler sequence may be located between the enhancer region and 3′ ITR. In one embodiment, a viral genome may comprise two filler sequences, the first filler sequence may be located between the 5′ ITR and intron region, and the second filler sequence may be located between the polyadenylation signal sequence region and MCS region. In one embodiment, a viral genome may comprise two filler sequences, the first filler sequence may be located between the 5′ ITR and intron region, and the second filler sequence may be located between the polyadenylation signal sequence region and exon region. In one embodiment, a viral genome may comprise two filler sequences, the first filler sequence may be located between the 5′ ITR and intron region, and the second filler sequence may be located between the polyadenylation signal sequence region and 3′ ITR. In one embodiment, a viral genome may comprise two filler sequences, the first filler sequence may be located between the 5′ ITR and intron region, and the second filler sequence may be located between the MCS region and exon region. In one embodiment, a viral genome may comprise two filler sequences, the first filler sequence may be located between the 5′ ITR and intron region, and the second filler sequence may be located between the MCS region and 3′ ITR. In one embodiment, a viral genome may comprise two filler sequences, the first filler sequence may be located between the 5′ ITR and intron region, and the second filler sequence may be located between the exon region and 3′ ITR.

In one embodiment, a viral genome may comprise two filler sequences, the first filler sequence may be located between the 5′ ITR and enhancer region, and the second filler sequence may be located between the promoter region and payload region. In one embodiment, a viral genome may comprise two filler sequences, the first filler sequence may be located between the 5′ ITR and enhancer region, and the second filler sequence may be located between the promoter region and intron region. In one embodiment, a viral genome may comprise two filler sequences, the first filler sequence may be located between the 5′ ITR and enhancer region, and the second filler sequence may be located between the promoter region and enhancer region. In one embodiment, a viral genome may comprise two filler sequences, the first filler sequence may be located between the 5′ ITR and enhancer region, and the second filler sequence may be located between the promoter region and polyadenylation signal sequence region. In one embodiment, a viral genome may comprise two filler sequences, the first filler sequence may be located between the 5′ ITR and enhancer region, and the second filler sequence may be located between the promoter region and MCS region. In one embodiment, a viral genome may comprise two filler sequences, the first filler sequence may be located between the 5′ ITR and enhancer region, and the second filler sequence may be located between the promoter region and exon region. In one embodiment, a viral genome may comprise two filler sequences, the first filler sequence may be located between the 5′ ITR and enhancer region, and the second filler sequence may be located between the promoter region and 3′ ITR. In one embodiment, a viral genome may comprise two filler sequences, the first filler sequence may be located between the 5′ ITR and enhancer region, and the second filler sequence may be located between the payload region and intron region. In one embodiment, a viral genome may comprise two filler sequences, the first filler sequence may be located between the 5′ ITR and enhancer region, and the second filler sequence may be located between the payload region and enhancer region. In one embodiment, a viral genome may comprise two filler sequences, the first filler sequence may be located between the 5′ ITR and enhancer region, and the second filler sequence may be located between the payload region and polyadenylation signal sequence region. In one embodiment, a viral genome may comprise two filler sequences, the first filler sequence may be located between the 5′ ITR and enhancer region, and the second filler sequence may be located between the payload region and MCS region. In one embodiment, a viral genome may comprise two filler sequences, the first filler sequence may be located between the 5′ ITR and enhancer region, and the second filler sequence may be located between the payload region and exon region. In one embodiment, a viral genome may comprise two filler sequences, the first filler sequence may be located between the 5′ ITR and enhancer region, and the second filler sequence may be located between the payload region and 3′ ITR. In one embodiment, a viral genome may comprise two filler sequences, the first filler sequence may be located between the 5′ ITR and enhancer region, and the second filler sequence may be located between the intron region and enhancer region. In one embodiment, a viral genome may comprise two filler sequences, the first filler sequence may be located between the 5′ ITR and enhancer region, and the second filler sequence may be located between the intron region and polyadenylation signal sequence region. In one embodiment, a viral genome may comprise two filler sequences, the first filler sequence may be located between the 5′ ITR and enhancer region, and the second filler sequence may be located between the intron region and MCS region. In one embodiment, a viral genome may comprise two filler sequences, the first filler sequence may be located between the 5′ ITR and enhancer region, and the second filler sequence may be located between the intron region and exon region. In one embodiment, a viral genome may comprise two filler sequences, the first filler sequence may be located between the 5′ ITR and enhancer region, and the second filler sequence may be located between the intron region and 3′ ITR. In one embodiment, a viral genome may comprise two filler sequences, the first filler sequence may be located between the 5′ ITR and enhancer region, and the second filler sequence may be located between the enhancer region and polyadenylation signal sequence region. In one embodiment, a viral genome may comprise two filler sequences, the first filler sequence may be located between the 5′ ITR and enhancer region, and the second filler sequence may be located between the enhancer region and MCS region. In one embodiment, a viral genome may comprise two filler sequences, the first filler sequence may be located between the 5′ ITR and enhancer region, and the second filler sequence may be located between the enhancer region and exon region. In one embodiment, a viral genome may comprise two filler sequences, the first filler sequence may be located between the 5′ ITR and enhancer region, and the second filler sequence may be located between the enhancer region and 3′ ITR. In one embodiment, a viral genome may comprise two filler sequences, the first filler sequence may be located between the 5′ ITR and enhancer region, and the second filler sequence may be located between the polyadenylation signal sequence region and MCS region. In one embodiment, a viral genome may comprise two filler sequences, the first filler sequence may be located between the 5′ ITR and enhancer region, and the second filler sequence may be located between the polyadenylation signal sequence region and exon region. In one embodiment, a viral genome may comprise two filler sequences, the first filler sequence may be located between the 5′ ITR and enhancer region, and the second filler sequence may be located between the polyadenylation signal sequence region and 3′ ITR. In one embodiment, a viral genome may comprise two filler sequences, the first filler sequence may be located between the 5′ ITR and enhancer region, and the second filler sequence may be located between the MCS region and exon region. In one embodiment, a viral genome may comprise two filler sequences, the first filler sequence may be located between the 5′ ITR and enhancer region, and the second filler sequence may be located between the MCS region and 3′ ITR. In one embodiment, a viral genome may comprise two filler sequences, the first filler sequence may be located between the 5′ ITR and enhancer region, and the second filler sequence may be located between the exon region and 3′ ITR.

In one embodiment, a viral genome may comprise two filler sequences, the first filler sequence may be located between the 5′ ITR and polyadenylation signal sequence region, and the second filler sequence may be located between the promoter region and payload region. In one embodiment, a viral genome may comprise two filler sequences, the first filler sequence may be located between the 5′ ITR and polyadenylation signal sequence region, and the second filler sequence may be located between the promoter region and intron region. In one embodiment, a viral genome may comprise two filler sequences, the first filler sequence may be located between the 5′ ITR and polyadenylation signal sequence region, and the second filler sequence may be located between the promoter region and enhancer region. In one embodiment, a viral genome may comprise two filler sequences, the first filler sequence may be located between the 5′ ITR and polyadenylation signal sequence region, and the second filler sequence may be located between the promoter region and polyadenylation signal sequence region. In one embodiment, a viral genome may comprise two filler sequences, the first filler sequence may be located between the 5′ ITR and polyadenylation signal sequence region, and the second filler sequence may be located between the promoter region and MCS region. In one embodiment, a viral genome may comprise two filler sequences, the first filler sequence may be located between the 5′ ITR and polyadenylation signal sequence region, and the second filler sequence may be located between the promoter region and exon region. In one embodiment, a viral genome may comprise two filler sequences, the first filler sequence may be located between the 5′ ITR and polyadenylation signal sequence region, and the second filler sequence may be located between the promoter region and 3′ ITR. In one embodiment, a viral genome may comprise two filler sequences, the first filler sequence may be located between the 5′ ITR and polyadenylation signal sequence region, and the second filler sequence may be located between the payload region and intron region. In one embodiment, a viral genome may comprise two filler sequences, the first filler sequence may be located between the 5′ ITR and polyadenylation signal sequence region, and the second filler sequence may be located between the payload region and enhancer region. In one embodiment, a viral genome may comprise two filler sequences, the first filler sequence may be located between the 5′ ITR and polyadenylation signal sequence region, and the second filler sequence may be located between the payload region and polyadenylation signal sequence region. In one embodiment, a viral genome may comprise two filler sequences, the first filler sequence may be located between the 5′ ITR and polyadenylation signal sequence region, and the second filler sequence may be located between the payload region and MCS region. In one embodiment, a viral genome may comprise two filler sequences, the first filler sequence may be located between the 5′ ITR and polyadenylation signal sequence region, and the second filler sequence may be located between the payload region and exon region. In one embodiment, a viral genome may comprise two filler sequences, the first filler sequence may be located between the 5′ ITR and polyadenylation signal sequence region, and the second filler sequence may be located between the payload region and 3′ ITR. In one embodiment, a viral genome may comprise two filler sequences, the first filler sequence may be located between the 5′ ITR and polyadenylation signal sequence region, and the second filler sequence may be located between the intron region and enhancer region. In one embodiment, a viral genome may comprise two filler sequences, the first filler sequence may be located between the 5′ ITR and polyadenylation signal sequence region, and the second filler sequence may be located between the intron region and polyadenylation signal sequence region. In one embodiment, a viral genome may comprise two filler sequences, the first filler sequence may be located between the 5′ ITR and polyadenylation signal sequence region, and the second filler sequence may be located between the intron region and MCS region. In one embodiment, a viral genome may comprise two filler sequences, the first filler sequence may be located between the 5′ ITR and polyadenylation signal sequence region, and the second filler sequence may be located between the intron region and exon region. In one embodiment, a viral genome may comprise two filler sequences, the first filler sequence may be located between the 5′ ITR and polyadenylation signal sequence region, and the second filler sequence may be located between the intron region and 3′ ITR. In one embodiment, a viral genome may comprise two filler sequences, the first filler sequence may be located between the 5′ ITR and polyadenylation signal sequence region, and the second filler sequence may be located between the enhancer region and polyadenylation signal sequence region. In one embodiment, a viral genome may comprise two filler sequences, the first filler sequence may be located between the 5′ ITR and polyadenylation signal sequence region, and the second filler sequence may be located between the enhancer region and MCS region. In one embodiment, a viral genome may comprise two filler sequences, the first filler sequence may be located between the 5′ ITR and polyadenylation signal sequence region, and the second filler sequence may be located between the enhancer region and exon region. In one embodiment, a viral genome may comprise two filler sequences, the first filler sequence may be located between the 5′ ITR and polyadenylation signal sequence region, and the second filler sequence may be located between the enhancer region and 3′ ITR. In one embodiment, a viral genome may comprise two filler sequences, the first filler sequence may be located between the 5′ ITR and polyadenylation signal sequence region, and the second filler sequence may be located between the polyadenylation signal sequence region and MCS region. In one embodiment, a viral genome may comprise two filler sequences, the first filler sequence may be located between the 5′ ITR and polyadenylation signal sequence region, and the second filler sequence may be located between the polyadenylation signal sequence region and exon region. In one embodiment, a viral genome may comprise two filler sequences, the first filler sequence may be located between the 5′ ITR and polyadenylation signal sequence region, and the second filler sequence may be located between the polyadenylation signal sequence region and 3′ ITR. In one embodiment, a viral genome may comprise two filler sequences, the first filler sequence may be located between the 5′ ITR and polyadenylation signal sequence region, and the second filler sequence may be located between the MCS region and exon region. In one embodiment, a viral genome may comprise two filler sequences, the first filler sequence may be located between the 5′ ITR and polyadenylation signal sequence region, and the second filler sequence may be located between the MCS region and 3′ ITR. In one embodiment, a viral genome may comprise two filler sequences, the first filler sequence may be located between the 5′ ITR and polyadenylation signal sequence region, and the second filler sequence may be located between the exon region and 3′ ITR.

In one embodiment, a viral genome may comprise two filler sequences, the first filler sequence may be located between the 5′ ITR and MCS region, and the second filler sequence may be located between the promoter region and payload region. In one embodiment, a viral genome may comprise two filler sequences, the first filler sequence may be located between the 5′ ITR and MCS region, and the second filler sequence may be located between the promoter region and intron region. In one embodiment, a viral genome may comprise two filler sequences, the first filler sequence may be located between the 5′ ITR and MCS region, and the second filler sequence may be located between the promoter region and enhancer region. In one embodiment, a viral genome may comprise two filler sequences, the first filler sequence may be located between the 5′ ITR and MCS region, and the second filler sequence may be located between the promoter region and polyadenylation signal sequence region. In one embodiment, a viral genome may comprise two filler sequences, the first filler sequence may be located between the 5′ ITR and MCS region, and the second filler sequence may be located between the promoter region and MCS region. In one embodiment, a viral genome may comprise two filler sequences, the first filler sequence may be located between the 5′ ITR and MCS region, and the second filler sequence may be located between the promoter region and exon region. In one embodiment, a viral genome may comprise two filler sequences, the first filler sequence may be located between the 5′ ITR and MCS region, and the second filler sequence may be located between the promoter region and 3′ ITR. In one embodiment, a viral genome may comprise two filler sequences, the first filler sequence may be located between the 5′ ITR and MCS region, and the second filler sequence may be located between the payload region and intron region. In one embodiment, a viral genome may comprise two filler sequences, the first filler sequence may be located between the 5′ ITR and MCS region, and the second filler sequence may be located between the payload region and enhancer region. In one embodiment, a viral genome may comprise two filler sequences, the first filler sequence may be located between the 5′ ITR and MCS region, and the second filler sequence may be located between the payload region and polyadenylation signal sequence region. In one embodiment, a viral genome may comprise two filler sequences, the first filler sequence may be located between the 5′ ITR and MCS region, and the second filler sequence may be located between the payload region and MCS region. In one embodiment, a viral genome may comprise two filler sequences, the first filler sequence may be located between the 5′ ITR and MCS region, and the second filler sequence may be located between the payload region and exon region. In one embodiment, a viral genome may comprise two filler sequences, the first filler sequence may be located between the 5′ ITR and MCS region, and the second filler sequence may be located between the payload region and 3′ ITR. In one embodiment, a viral genome may comprise two filler sequences, the first filler sequence may be located between the 5′ ITR and MCS region, and the second filler sequence may be located between the intron region and enhancer region. In one embodiment, a viral genome may comprise two filler sequences, the first filler sequence may be located between the 5′ ITR and MCS region, and the second filler sequence may be located between the intron region and polyadenylation signal sequence region. In one embodiment, a viral genome may comprise two filler sequences, the first filler sequence may be located between the 5′ ITR and MCS region, and the second filler sequence may be located between the intron region and MCS region. In one embodiment, a viral genome may comprise two filler sequences, the first filler sequence may be located between the 5′ ITR and MCS region, and the second filler sequence may be located between the intron region and exon region. In one embodiment, a viral genome may comprise two filler sequences, the first filler sequence may be located between the 5′ ITR and MCS region, and the second filler sequence may be located between the intron region and 3′ ITR. In one embodiment, a viral genome may comprise two filler sequences, the first filler sequence may be located between the 5′ ITR and MCS region, and the second filler sequence may be located between the enhancer region and polyadenylation signal sequence region. In one embodiment, a viral genome may comprise two filler sequences, the first filler sequence may be located between the 5′ ITR and MCS region, and the second filler sequence may be located between the enhancer region and MCS region. In one embodiment, a viral genome may comprise two filler sequences, the first filler sequence may be located between the 5′ ITR and MCS region, and the second filler sequence may be located between the enhancer region and exon region. In one embodiment, a viral genome may comprise two filler sequences, the first filler sequence may be located between the 5′ ITR and MCS region, and the second filler sequence may be located between the enhancer region and 3′ ITR. In one embodiment, a viral genome may comprise two filler sequences, the first filler sequence may be located between the 5′ ITR and MCS region, and the second filler sequence may be located between the polyadenylation signal sequence region and MCS region. In one embodiment, a viral genome may comprise two filler sequences, the first filler sequence may be located between the 5′ ITR and MCS region, and the second filler sequence may be located between the polyadenylation signal sequence region and exon region. In one embodiment, a viral genome may comprise two filler sequences, the first filler sequence may be located between the 5′ ITR and MCS region, and the second filler sequence may be located between the polyadenylation signal sequence region and 3′ ITR. In one embodiment, a viral genome may comprise two filler sequences, the first filler sequence may be located between the 5′ ITR and MCS region, and the second filler sequence may be located between the MCS region and exon region. In one embodiment, a viral genome may comprise two filler sequences, the first filler sequence may be located between the 5′ ITR and MCS region, and the second filler sequence may be located between the MCS region and 3′ ITR. In one embodiment, a viral genome may comprise two filler sequences, the first filler sequence may be located between the 5′ ITR and MCS region, and the second filler sequence may be located between the exon region and 3′ ITR.

In one embodiment, a viral genome may comprise two filler sequences, the first filler sequence may be located between the 5′ ITR and exon region, and the second filler sequence may be located between the promoter region and payload region. In one embodiment, a viral genome may comprise two filler sequences, the first filler sequence may be located between the 5′ ITR and exon region, and the second filler sequence may be located between the promoter region and intron region. In one embodiment, a viral genome may comprise two filler sequences, the first filler sequence may be located between the 5′ ITR and exon region, and the second filler sequence may be located between the promoter region and enhancer region. In one embodiment, a viral genome may comprise two filler sequences, the first filler sequence may be located between the 5′ ITR and exon region, and the second filler sequence may be located between the promoter region and polyadenylation signal sequence region. In one embodiment, a viral genome may comprise two filler sequences, the first filler sequence may be located between the 5′ ITR and exon region, and the second filler sequence may be located between the promoter region and MCS region. In one embodiment, a viral genome may comprise two filler sequences, the first filler sequence may be located between the 5′ ITR and exon region, and the second filler sequence may be located between the promoter region and exon region. In one embodiment, a viral genome may comprise two filler sequences, the first filler sequence may be located between the 5′ ITR and exon region, and the second filler sequence may be located between the promoter region and 3′ ITR. In one embodiment, a viral genome may comprise two filler sequences, the first filler sequence may be located between the 5′ ITR and exon region, and the second filler sequence may be located between the payload region and intron region. In one embodiment, a viral genome may comprise two filler sequences, the first filler sequence may be located between the 5′ ITR and exon region, and the second filler sequence may be located between the payload region and enhancer region. In one embodiment, a viral genome may comprise two filler sequences, the first filler sequence may be located between the 5′ ITR and exon region, and the second filler sequence may be located between the payload region and polyadenylation signal sequence region. In one embodiment, a viral genome may comprise two filler sequences, the first filler sequence may be located between the 5′ ITR and exon region, and the second filler sequence may be located between the payload region and MCS region. In one embodiment, a viral genome may comprise two filler sequences, the first filler sequence may be located between the 5′ ITR and exon region, and the second filler sequence may be located between the payload region and exon region. In one embodiment, a viral genome may comprise two filler sequences, the first filler sequence may be located between the 5′ ITR and exon region, and the second filler sequence may be located between the payload region and 3′ ITR. In one embodiment, a viral genome may comprise two filler sequences, the first filler sequence may be located between the 5′ ITR and exon region, and the second filler sequence may be located between the intron region and enhancer region. In one embodiment, a viral genome may comprise two filler sequences, the first filler sequence may be located between the 5′ ITR and exon region, and the second filler sequence may be located between the intron region and polyadenylation signal sequence region. In one embodiment, a viral genome may comprise two filler sequences, the first filler sequence may be located between the 5′ ITR and exon region, and the second filler sequence may be located between the intron region and MCS region. In one embodiment, a viral genome may comprise two filler sequences, the first filler sequence may be located between the 5′ ITR and exon region, and the second filler sequence may be located between the intron region and exon region. In one embodiment, a viral genome may comprise two filler sequences, the first filler sequence may be located between the 5′ ITR and exon region, and the second filler sequence may be located between the intron region and 3′ ITR. In one embodiment, a viral genome may comprise two filler sequences, the first filler sequence may be located between the 5′ ITR and exon region, and the second filler sequence may be located between the enhancer region and polyadenylation signal sequence region. In one embodiment, a viral genome may comprise two filler sequences, the first filler sequence may be located between the 5′ ITR and exon region, and the second filler sequence may be located between the enhancer region and MCS region. In one embodiment, a viral genome may comprise two filler sequences, the first filler sequence may be located between the 5′ ITR and exon region, and the second filler sequence may be located between the enhancer region and exon region. In one embodiment, a viral genome may comprise two filler sequences, the first filler sequence may be located between the 5′ ITR and exon region, and the second filler sequence may be located between the enhancer region and 3′ ITR. In one embodiment, a viral genome may comprise two filler sequences, the first filler sequence may be located between the 5′ ITR and exon region, and the second filler sequence may be located between the polyadenylation signal sequence region and MCS region. In one embodiment, a viral genome may comprise two filler sequences, the first filler sequence may be located between the 5′ ITR and exon region, and the second filler sequence may be located between the polyadenylation signal sequence region and exon region. In one embodiment, a viral genome may comprise two filler sequences, the first filler sequence may be located between the 5′ ITR and exon region, and the second filler sequence may be located between the polyadenylation signal sequence region and 3′ ITR. In one embodiment, a viral genome may comprise two filler sequences, the first filler sequence may be located between the 5′ ITR and exon region, and the second filler sequence may be located between the MCS region and exon region. In one embodiment, a viral genome may comprise two filler sequences, the first filler sequence may be located between the 5′ ITR and exon region, and the second filler sequence may be located between the MCS region and 3′ ITR. In one embodiment, a viral genome may comprise two filler sequences, the first filler sequence may be located between the 5′ ITR and exon region, and the second filler sequence may be located between the exon region and 3′ ITR.

In one embodiment, a viral genome may comprise two filler sequences, the first filler sequence may be located between the promoter region and payload region, and the second filler sequence may be located between the payload region and intron region. In one embodiment, a viral genome may comprise two filler sequences, the first filler sequence may be located between the promoter region and payload region, and the second filler sequence may be located between the payload region and enhancer region. In one embodiment, a viral genome may comprise two filler sequences, the first filler sequence may be located between the promoter region and payload region, and the second filler sequence may be located between the payload region and polyadenylation signal sequence region. In one embodiment, a viral genome may comprise two filler sequences, the first filler sequence may be located between the promoter region and payload region, and the second filler sequence may be located between the payload region and MCS region. In one embodiment, a viral genome may comprise two filler sequences, the first filler sequence may be located between the promoter region and payload region, and the second filler sequence may be located between the payload region and exon region. In one embodiment, a viral genome may comprise two filler sequences, the first filler sequence may be located between the promoter region and payload region, and the second filler sequence may be located between the payload region and 3′ ITR. In one embodiment, a viral genome may comprise two filler sequences, the first filler sequence may be located between the promoter region and payload region, and the second filler sequence may be located between the intron region and enhancer region. In one embodiment, a viral genome may comprise two filler sequences, the first filler sequence may be located between the promoter region and payload region, and the second filler sequence may be located between the intron region and polyadenylation signal sequence region. In one embodiment, a viral genome may comprise two filler sequences, the first filler sequence may be located between the promoter region and payload region, and the second filler sequence may be located between the intron region and MCS region. In one embodiment, a viral genome may comprise two filler sequences, the first filler sequence may be located between the promoter region and payload region, and the second filler sequence may be located between the intron region and exon region. In one embodiment, a viral genome may comprise two filler sequences, the first filler sequence may be located between the promoter region and payload region, and the second filler sequence may be located between the intron region and 3′ ITR. In one embodiment, a viral genome may comprise two filler sequences, the first filler sequence may be located between the promoter region and payload region, and the second filler sequence may be located between the enhancer region and polyadenylation signal sequence region. In one embodiment, a viral genome may comprise two filler sequences, the first filler sequence may be located between the promoter region and payload region, and the second filler sequence may be located between the enhancer region and MCS region. In one embodiment, a viral genome may comprise two filler sequences, the first filler sequence may be located between the promoter region and payload region, and the second filler sequence may be located between the enhancer region and exon region. In one embodiment, a viral genome may comprise two filler sequences, the first filler sequence may be located between the promoter region and payload region, and the second filler sequence may be located between the enhancer region and 3′ ITR. In one embodiment, a viral genome may comprise two filler sequences, the first filler sequence may be located between the promoter region and payload region, and the second filler sequence may be located between the polyadenylation signal sequence region and MCS region. In one embodiment, a viral genome may comprise two filler sequences, the first filler sequence may be located between the promoter region and payload region, and the second filler sequence may be located between the polyadenylation signal sequence region and exon region. In one embodiment, a viral genome may comprise two filler sequences, the first filler sequence may be located between the promoter region and payload region, and the second filler sequence may be located between the polyadenylation signal sequence region and 3′ ITR. In one embodiment, a viral genome may comprise two filler sequences, the first filler sequence may be located between the promoter region and payload region, and the second filler sequence may be located between the MCS region and exon region. In one embodiment, a viral genome may comprise two filler sequences, the first filler sequence may be located between the promoter region and payload region, and the second filler sequence may be located between the MCS region and 3′ ITR. In one embodiment, a viral genome may comprise two filler sequences, the first filler sequence may be located between the promoter region and payload region, and the second filler sequence may be located between the exon region and 3′ ITR.

In one embodiment, a viral genome may comprise two filler sequences, the first filler sequence may be located between the promoter region and intron region, and the second filler sequence may be located between the payload region and intron region. In one embodiment, a viral genome may comprise two filler sequences, the first filler sequence may be located between the promoter region and intron region, and the second filler sequence may be located between the payload region and enhancer region. In one embodiment, a viral genome may comprise two filler sequences, the first filler sequence may be located between the promoter region and intron region, and the second filler sequence may be located between the payload region and polyadenylation signal sequence region. In one embodiment, a viral genome may comprise two filler sequences, the first filler sequence may be located between the promoter region and intron region, and the second filler sequence may be located between the payload region and MCS region. In one embodiment, a viral genome may comprise two filler sequences, the first filler sequence may be located between the promoter region and intron region, and the second filler sequence may be located between the payload region and exon region. In one embodiment, a viral genome may comprise two filler sequences, the first filler sequence may be located between the promoter region and intron region, and the second filler sequence may be located between the payload region and 3′ ITR. In one embodiment, a viral genome may comprise two filler sequences, the first filler sequence may be located between the promoter region and intron region, and the second filler sequence may be located between the intron region and enhancer region. In one embodiment, a viral genome may comprise two filler sequences, the first filler sequence may be located between the promoter region and intron region, and the second filler sequence may be located between the intron region and polyadenylation signal sequence region. In one embodiment, a viral genome may comprise two filler sequences, the first filler sequence may be located between the promoter region and intron region, and the second filler sequence may be located between the intron region and MCS region. In one embodiment, a viral genome may comprise two filler sequences, the first filler sequence may be located between the promoter region and intron region, and the second filler sequence may be located between the intron region and exon region. In one embodiment, a viral genome may comprise two filler sequences, the first filler sequence may be located between the promoter region and intron region, and the second filler sequence may be located between the intron region and 3′ ITR. In one embodiment, a viral genome may comprise two filler sequences, the first filler sequence may be located between the promoter region and intron region, and the second filler sequence may be located between the enhancer region and polyadenylation signal sequence region. In one embodiment, a viral genome may comprise two filler sequences, the first filler sequence may be located between the promoter region and intron region, and the second filler sequence may be located between the enhancer region and MCS region. In one embodiment, a viral genome may comprise two filler sequences, the first filler sequence may be located between the promoter region and intron region, and the second filler sequence may be located between the enhancer region and exon region. In one embodiment, a viral genome may comprise two filler sequences, the first filler sequence may be located between the promoter region and intron region, and the second filler sequence may be located between the enhancer region and 3′ ITR. In one embodiment, a viral genome may comprise two filler sequences, the first filler sequence may be located between the promoter region and intron region, and the second filler sequence may be located between the polyadenylation signal sequence region and MCS region. In one embodiment, a viral genome may comprise two filler sequences, the first filler sequence may be located between the promoter region and intron region, and the second filler sequence may be located between the polyadenylation signal sequence region and exon region. In one embodiment, a viral genome may comprise two filler sequences, the first filler sequence may be located between the promoter region and intron region, and the second filler sequence may be located between the polyadenylation signal sequence region and 3′ ITR. In one embodiment, a viral genome may comprise two filler sequences, the first filler sequence may be located between the promoter region and intron region, and the second filler sequence may be located between the MCS region and exon region. In one embodiment, a viral genome may comprise two filler sequences, the first filler sequence may be located between the promoter region and intron region, and the second filler sequence may be located between the MCS region and 3′ ITR. In one embodiment, a viral genome may comprise two filler sequences, the first filler sequence may be located between the promoter region and intron region, and the second filler sequence may be located between the exon region and 3′ ITR.

In one embodiment, a viral genome may comprise two filler sequences, the first filler sequence may be located between the promoter region and enhancer region, and the second filler sequence may be located between the payload region and intron region. In one embodiment, a viral genome may comprise two filler sequences, the first filler sequence may be located between the promoter region and enhancer region, and the second filler sequence may be located between the payload region and enhancer region. In one embodiment, a viral genome may comprise two filler sequences, the first filler sequence may be located between the promoter region and enhancer region, and the second filler sequence may be located between the payload region and polyadenylation signal sequence region. In one embodiment, a viral genome may comprise two filler sequences, the first filler sequence may be located between the promoter region and enhancer region, and the second filler sequence may be located between the payload region and MCS region. In one embodiment, a viral genome may comprise two filler sequences, the first filler sequence may be located between the promoter region and enhancer region, and the second filler sequence may be located between the payload region and exon region. In one embodiment, a viral genome may comprise two filler sequences, the first filler sequence may be located between the promoter region and enhancer region, and the second filler sequence may be located between the payload region and 3′ ITR. In one embodiment, a viral genome may comprise two filler sequences, the first filler sequence may be located between the promoter region and enhancer region, and the second filler sequence may be located between the intron region and enhancer region. In one embodiment, a viral genome may comprise two filler sequences, the first filler sequence may be located between the promoter region and enhancer region, and the second filler sequence may be located between the intron region and polyadenylation signal sequence region. In one embodiment, a viral genome may comprise two filler sequences, the first filler sequence may be located between the promoter region and enhancer region, and the second filler sequence may be located between the intron region and MCS region. In one embodiment, a viral genome may comprise two filler sequences, the first filler sequence may be located between the promoter region and enhancer region, and the second filler sequence may be located between the intron region and exon region. In one embodiment, a viral genome may comprise two filler sequences, the first filler sequence may be located between the promoter region and enhancer region, and the second filler sequence may be located between the intron region and 3′ ITR. In one embodiment, a viral genome may comprise two filler sequences, the first filler sequence may be located between the promoter region and enhancer region, and the second filler sequence may be located between the enhancer region and polyadenylation signal sequence region. In one embodiment, a viral genome may comprise two filler sequences, the first filler sequence may be located between the promoter region and enhancer region, and the second filler sequence may be located between the enhancer region and MCS region. In one embodiment, a viral genome may comprise two filler sequences, the first filler sequence may be located between the promoter region and enhancer region, and the second filler sequence may be located between the enhancer region and exon region. In one embodiment, a viral genome may comprise two filler sequences, the first filler sequence may be located between the promoter region and enhancer region, and the second filler sequence may be located between the enhancer region and 3′ ITR. In one embodiment, a viral genome may comprise two filler sequences, the first filler sequence may be located between the promoter region and enhancer region, and the second filler sequence may be located between the polyadenylation signal sequence region and MCS region. In one embodiment, a viral genome may comprise two filler sequences, the first filler sequence may be located between the promoter region and enhancer region, and the second filler sequence may be located between the polyadenylation signal sequence region and exon region. In one embodiment, a viral genome may comprise two filler sequences, the first filler sequence may be located between the promoter region and enhancer region, and the second filler sequence may be located between the polyadenylation signal sequence region and 3′ ITR. In one embodiment, a viral genome may comprise two filler sequences, the first filler sequence may be located between the promoter region and enhancer region, and the second filler sequence may be located between the MCS region and exon region. In one embodiment, a viral genome may comprise two filler sequences, the first filler sequence may be located between the promoter region and enhancer region, and the second filler sequence may be located between the MCS region and 3′ ITR. In one embodiment, a viral genome may comprise two filler sequences, the first filler sequence may be located between the promoter region and enhancer region, and the second filler sequence may be located between the exon region and 3′ ITR.

In one embodiment, a viral genome may comprise two filler sequences, the first filler sequence may be located between the promoter region and polyadenylation signal sequence region, and the second filler sequence may be located between the payload region and intron region. In one embodiment, a viral genome may comprise two filler sequences, the first filler sequence may be located between the promoter region and polyadenylation signal sequence region, and the second filler sequence may be located between the payload region and enhancer region. In one embodiment, a viral genome may comprise two filler sequences, the first filler sequence may be located between the promoter region and polyadenylation signal sequence region, and the second filler sequence may be located between the payload region and polyadenylation signal sequence region. In one embodiment, a viral genome may comprise two filler sequences, the first filler sequence may be located between the promoter region and polyadenylation signal sequence region, and the second filler sequence may be located between the payload region and MCS region. In one embodiment, a viral genome may comprise two filler sequences, the first filler sequence may be located between the promoter region and polyadenylation signal sequence region, and the second filler sequence may be located between the payload region and exon region. In one embodiment, a viral genome may comprise two filler sequences, the first filler sequence may be located between the promoter region and polyadenylation signal sequence region, and the second filler sequence may be located between the payload region and 3′ ITR. In one embodiment, a viral genome may comprise two filler sequences, the first filler sequence may be located between the promoter region and polyadenylation signal sequence region, and the second filler sequence may be located between the intron region and enhancer region. In one embodiment, a viral genome may comprise two filler sequences, the first filler sequence may be located between the promoter region and polyadenylation signal sequence region, and the second filler sequence may be located between the intron region and polyadenylation signal sequence region. In one embodiment, a viral genome may comprise two filler sequences, the first filler sequence may be located between the promoter region and polyadenylation signal sequence region, and the second filler sequence may be located between the intron region and MCS region. In one embodiment, a viral genome may comprise two filler sequences, the first filler sequence may be located between the promoter region and polyadenylation signal sequence region, and the second filler sequence may be located between the intron region and exon region. In one embodiment, a viral genome may comprise two filler sequences, the first filler sequence may be located between the promoter region and polyadenylation signal sequence region, and the second filler sequence may be located between the intron region and 3′ ITR. In one embodiment, a viral genome may comprise two filler sequences, the first filler sequence may be located between the promoter region and polyadenylation signal sequence region, and the second filler sequence may be located between the enhancer region and polyadenylation signal sequence region. In one embodiment, a viral genome may comprise two filler sequences, the first filler sequence may be located between the promoter region and polyadenylation signal sequence region, and the second filler sequence may be located between the enhancer region and MCS region. In one embodiment, a viral genome may comprise two filler sequences, the first filler sequence may be located between the promoter region and polyadenylation signal sequence region, and the second filler sequence may be located between the enhancer region and exon region. In one embodiment, a viral genome may comprise two filler sequences, the first filler sequence may be located between the promoter region and polyadenylation signal sequence region, and the second filler sequence may be located between the enhancer region and 3′ ITR. In one embodiment, a viral genome may comprise two filler sequences, the first filler sequence may be located between the promoter region and polyadenylation signal sequence region, and the second filler sequence may be located between the polyadenylation signal sequence region and MCS region. In one embodiment, a viral genome may comprise two filler sequences, the first filler sequence may be located between the promoter region and polyadenylation signal sequence region, and the second filler sequence may be located between the polyadenylation signal sequence region and exon region. In one embodiment, a viral genome may comprise two filler sequences, the first filler sequence may be located between the promoter region and polyadenylation signal sequence region, and the second filler sequence may be located between the polyadenylation signal sequence region and 3′ ITR. In one embodiment, a viral genome may comprise two filler sequences, the first filler sequence may be located between the promoter region and polyadenylation signal sequence region, and the second filler sequence may be located between the MCS region and exon region. In one embodiment, a viral genome may comprise two filler sequences, the first filler sequence may be located between the promoter region and polyadenylation signal sequence region, and the second filler sequence may be located between the MCS region and 3′ ITR. In one embodiment, a viral genome may comprise two filler sequences, the first filler sequence may be located between the promoter region and polyadenylation signal sequence region, and the second filler sequence may be located between the exon region and 3′ ITR.

In one embodiment, a viral genome may comprise two filler sequences, the first filler sequence may be located between the promoter region and exon region, and the second filler sequence may be located between the payload region and intron region. In one embodiment, a viral genome may comprise two filler sequences, the first filler sequence may be located between the promoter region and exon region, and the second filler sequence may be located between the payload region and enhancer region. In one embodiment, a viral genome may comprise two filler sequences, the first filler sequence may be located between the promoter region and exon region, and the second filler sequence may be located between the payload region and polyadenylation signal sequence region. In one embodiment, a viral genome may comprise two filler sequences, the first filler sequence may be located between the promoter region and exon region, and the second filler sequence may be located between the payload region and MCS region. In one embodiment, a viral genome may comprise two filler sequences, the first filler sequence may be located between the promoter region and exon region, and the second filler sequence may be located between the payload region and exon region. In one embodiment, a viral genome may comprise two filler sequences, the first filler sequence may be located between the promoter region and exon region, and the second filler sequence may be located between the payload region and 3′ ITR. In one embodiment, a viral genome may comprise two filler sequences, the first filler sequence may be located between the promoter region and exon region, and the second filler sequence may be located between the intron region and enhancer region. In one embodiment, a viral genome may comprise two filler sequences, the first filler sequence may be located between the promoter region and exon region, and the second filler sequence may be located between the intron region and polyadenylation signal sequence region. In one embodiment, a viral genome may comprise two filler sequences, the first filler sequence may be located between the promoter region and exon region, and the second filler sequence may be located between the intron region and MCS region. In one embodiment, a viral genome may comprise two filler sequences, the first filler sequence may be located between the promoter region and exon region, and the second filler sequence may be located between the intron region and exon region. In one embodiment, a viral genome may comprise two filler sequences, the first filler sequence may be located between the promoter region and exon region, and the second filler sequence may be located between the intron region and 3′ ITR. In one embodiment, a viral genome may comprise two filler sequences, the first filler sequence may be located between the promoter region and exon region, and the second filler sequence may be located between the enhancer region and polyadenylation signal sequence region. In one embodiment, a viral genome may comprise two filler sequences, the first filler sequence may be located between the promoter region and exon region, and the second filler sequence may be located between the enhancer region and MCS region. In one embodiment, a viral genome may comprise two filler sequences, the first filler sequence may be located between the promoter region and exon region, and the second filler sequence may be located between the enhancer region and exon region. In one embodiment, a viral genome may comprise two filler sequences, the first filler sequence may be located between the promoter region and exon region, and the second filler sequence may be located between the enhancer region and 3′ ITR. In one embodiment, a viral genome may comprise two filler sequences, the first filler sequence may be located between the promoter region and exon region, and the second filler sequence may be located between the polyadenylation signal sequence region and MCS region. In one embodiment, a viral genome may comprise two filler sequences, the first filler sequence may be located between the promoter region and exon region, and the second filler sequence may be located between the polyadenylation signal sequence region and exon region. In one embodiment, a viral genome may comprise two filler sequences, the first filler sequence may be located between the promoter region and exon region, and the second filler sequence may be located between the polyadenylation signal sequence region and 3′ ITR. In one embodiment, a viral genome may comprise two filler sequences, the first filler sequence may be located between the promoter region and exon region, and the second filler sequence may be located between the MCS region and exon region. In one embodiment, a viral genome may comprise two filler sequences, the first filler sequence may be located between the promoter region and exon region, and the second filler sequence may be located between the MCS region and 3′ ITR. In one embodiment, a viral genome may comprise two filler sequences, the first filler sequence may be located between the promoter region and exon region, and the second filler sequence may be located between the exon region and 3′ ITR.

In one embodiment, a viral genome may comprise two filler sequences, the first filler sequence may be located between the promoter region and MCS region, and the second filler sequence may be located between the payload region and intron region. In one embodiment, a viral genome may comprise two filler sequences, the first filler sequence may be located between the promoter region and MCS region, and the second filler sequence may be located between the payload region and enhancer region. In one embodiment, a viral genome may comprise two filler sequences, the first filler sequence may be located between the promoter region and MCS region, and the second filler sequence may be located between the payload region and polyadenylation signal sequence region. In one embodiment, a viral genome may comprise two filler sequences, the first filler sequence may be located between the promoter region and MCS region, and the second filler sequence may be located between the payload region and MCS region. In one embodiment, a viral genome may comprise two filler sequences, the first filler sequence may be located between the promoter region and MCS region, and the second filler sequence may be located between the payload region and exon region. In one embodiment, a viral genome may comprise two filler sequences, the first filler sequence may be located between the promoter region and MCS region, and the second filler sequence may be located between the payload region and 3′ ITR. In one embodiment, a viral genome may comprise two filler sequences, the first filler sequence may be located between the promoter region and MCS region, and the second filler sequence may be located between the intron region and enhancer region. In one embodiment, a viral genome may comprise two filler sequences, the first filler sequence may be located between the promoter region and MCS region, and the second filler sequence may be located between the intron region and polyadenylation signal sequence region. In one embodiment, a viral genome may comprise two filler sequences, the first filler sequence may be located between the promoter region and MCS region, and the second filler sequence may be located between the intron region and MCS region. In one embodiment, a viral genome may comprise two filler sequences, the first filler sequence may be located between the promoter region and MCS region, and the second filler sequence may be located between the intron region and exon region. In one embodiment, a viral genome may comprise two filler sequences, the first filler sequence may be located between the promoter region and MCS region, and the second filler sequence may be located between the intron region and 3′ ITR. In one embodiment, a viral genome may comprise two filler sequences, the first filler sequence may be located between the promoter region and MCS region, and the second filler sequence may be located between the enhancer region and polyadenylation signal sequence region. In one embodiment, a viral genome may comprise two filler sequences, the first filler sequence may be located between the promoter region and MCS region, and the second filler sequence may be located between the enhancer region and MCS region. In one embodiment, a viral genome may comprise two filler sequences, the first filler sequence may be located between the promoter region and MCS region, and the second filler sequence may be located between the enhancer region and exon region. In one embodiment, a viral genome may comprise two filler sequences, the first filler sequence may be located between the promoter region and MCS region, and the second filler sequence may be located between the enhancer region and 3′ ITR. In one embodiment, a viral genome may comprise two filler sequences, the first filler sequence may be located between the promoter region and MCS region, and the second filler sequence may be located between the polyadenylation signal sequence region and MCS region. In one embodiment, a viral genome may comprise two filler sequences, the first filler sequence may be located between the promoter region and MCS region, and the second filler sequence may be located between the polyadenylation signal sequence region and exon region. In one embodiment, a viral genome may comprise two filler sequences, the first filler sequence may be located between the promoter region and MCS region, and the second filler sequence may be located between the polyadenylation signal sequence region and 3′ ITR. In one embodiment, a viral genome may comprise two filler sequences, the first filler sequence may be located between the promoter region and MCS region, and the second filler sequence may be located between the MCS region and exon region. In one embodiment, a viral genome may comprise two filler sequences, the first filler sequence may be located between the promoter region and MCS region, and the second filler sequence may be located between the MCS region and 3′ ITR. In one embodiment, a viral genome may comprise two filler sequences, the first filler sequence may be located between the promoter region and MCS region, and the second filler sequence may be located between the exon region and 3′ ITR.

In one embodiment, a viral genome may comprise two filler sequences, the first filler sequence may be located between the promoter region and 3′ITR, and the second filler sequence may be located between the payload region and intron region. In one embodiment, a viral genome may comprise two filler sequences, the first filler sequence may be located between the promoter region and 3′ITR, and the second filler sequence may be located between the payload region and enhancer region. In one embodiment, a viral genome may comprise two filler sequences, the first filler sequence may be located between the promoter region and 3′ITR, and the second filler sequence may be located between the payload region and polyadenylation signal sequence region. In one embodiment, a viral genome may comprise two filler sequences, the first filler sequence may be located between the promoter region and 3′ITR, and the second filler sequence may be located between the payload region and MCS region. In one embodiment, a viral genome may comprise two filler sequences, the first filler sequence may be located between the promoter region and 3′ITR, and the second filler sequence may be located between the payload region and exon region. In one embodiment, a viral genome may comprise two filler sequences, the first filler sequence may be located between the promoter region and 3′ITR, and the second filler sequence may be located between the payload region and 3′ ITR. In one embodiment, a viral genome may comprise two filler sequences, the first filler sequence may be located between the promoter region and 3′ITR, and the second filler sequence may be located between the intron region and enhancer region. In one embodiment, a viral genome may comprise two filler sequences, the first filler sequence may be located between the promoter region and 3′ITR, and the second filler sequence may be located between the intron region and polyadenylation signal sequence region. In one embodiment, a viral genome may comprise two filler sequences, the first filler sequence may be located between the promoter region and 3′ITR, and the second filler sequence may be located between the intron region and MCS region. In one embodiment, a viral genome may comprise two filler sequences, the first filler sequence may be located between the promoter region and 3′ITR, and the second filler sequence may be located between the intron region and exon region. In one embodiment, a viral genome may comprise two filler sequences, the first filler sequence may be located between the promoter region and 3′ITR, and the second filler sequence may be located between the intron region and 3′ ITR. In one embodiment, a viral genome may comprise two filler sequences, the first filler sequence may be located between the promoter region and 3′ITR, and the second filler sequence may be located between the enhancer region and polyadenylation signal sequence region. In one embodiment, a viral genome may comprise two filler sequences, the first filler sequence may be located between the promoter region and 3′ITR, and the second filler sequence may be located between the enhancer region and MCS region. In one embodiment, a viral genome may comprise two filler sequences, the first filler sequence may be located between the promoter region and 3′ITR, and the second filler sequence may be located between the enhancer region and exon region. In one embodiment, a viral genome may comprise two filler sequences, the first filler sequence may be located between the promoter region and 3′ITR, and the second filler sequence may be located between the enhancer region and 3′ ITR. In one embodiment, a viral genome may comprise two filler sequences, the first filler sequence may be located between the promoter region and 3′ITR, and the second filler sequence may be located between the polyadenylation signal sequence region and MCS region. In one embodiment, a viral genome may comprise two filler sequences, the first filler sequence may be located between the promoter region and 3′ITR, and the second filler sequence may be located between the polyadenylation signal sequence region and exon region. In one embodiment, a viral genome may comprise two filler sequences, the first filler sequence may be located between the promoter region and 3′ITR, and the second filler sequence may be located between the polyadenylation signal sequence region and 3′ ITR. In one embodiment, a viral genome may comprise two filler sequences, the first filler sequence may be located between the promoter region and 3′ITR, and the second filler sequence may be located between the MCS region and exon region. In one embodiment, a viral genome may comprise two filler sequences, the first filler sequence may be located between the promoter region and 3′ITR, and the second filler sequence may be located between the MCS region and 3′ ITR. In one embodiment, a viral genome may comprise two filler sequences, the first filler sequence may be located between the promoter region and 3′ITR, and the second filler sequence may be located between the exon region and 3′ ITR.

In one embodiment, a viral genome may comprise two filler sequences, the first filler sequence may be located between the payload region and intron region, and the second filler sequence may be located between the intron region and enhancer region. In one embodiment, a viral genome may comprise two filler sequences, the first filler sequence may be located between the payload region and intron region, and the second filler sequence may be located between the intron region and polyadenylation signal sequence region. In one embodiment, a viral genome may comprise two filler sequences, the first filler sequence may be located between the payload region and intron region, and the second filler sequence may be located between the intron region and MCS region. In one embodiment, a viral genome may comprise two filler sequences, the first filler sequence may be located between the payload region and intron region, and the second filler sequence may be located between the intron region and exon region. In one embodiment, a viral genome may comprise two filler sequences, the first filler sequence may be located between the payload region and intron region, and the second filler sequence may be located between the intron region and 3′ ITR. In one embodiment, a viral genome may comprise two filler sequences, the first filler sequence may be located between the payload region and intron region, and the second filler sequence may be located between the enhancer region and polyadenylation signal sequence region. In one embodiment, a viral genome may comprise two filler sequences, the first filler sequence may be located between the payload region and intron region, and the second filler sequence may be located between the enhancer region and MCS region. In one embodiment, a viral genome may comprise two filler sequences, the first filler sequence may be located between the payload region and intron region, and the second filler sequence may be located between the enhancer region and exon region. In one embodiment, a viral genome may comprise two filler sequences, the first filler sequence may be located between the payload region and intron region, and the second filler sequence may be located between the enhancer region and 3′ ITR. In one embodiment, a viral genome may comprise two filler sequences, the first filler sequence may be located between the payload region and intron region, and the second filler sequence may be located between the polyadenylation signal sequence region and MCS region. In one embodiment, a viral genome may comprise two filler sequences, the first filler sequence may be located between the payload region and intron region, and the second filler sequence may be located between the polyadenylation signal sequence region and exon region. In one embodiment, a viral genome may comprise two filler sequences, the first filler sequence may be located between the payload region and intron region, and the second filler sequence may be located between the polyadenylation signal sequence region and 3′ ITR. In one embodiment, a viral genome may comprise two filler sequences, the first filler sequence may be located between the payload region and intron region, and the second filler sequence may be located between the MCS region and exon region. In one embodiment, a viral genome may comprise two filler sequences, the first filler sequence may be located between the payload region and intron region, and the second filler sequence may be located between the MCS region and 3′ ITR. In one embodiment, a viral genome may comprise two filler sequences, the first filler sequence may be located between the payload region and intron region, and the second filler sequence may be located between the exon region and 3′ ITR.

In one embodiment, a viral genome may comprise two filler sequences, the first filler sequence may be located between the payload region and enhancer region, and the second filler sequence may be located between the intron region and enhancer region. In one embodiment, a viral genome may comprise two filler sequences, the first filler sequence may be located between the payload region and enhancer region, and the second filler sequence may be located between the intron region and polyadenylation signal sequence region. In one embodiment, a viral genome may comprise two filler sequences, the first filler sequence may be located between the payload region and enhancer region, and the second filler sequence may be located between the intron region and MCS region. In one embodiment, a viral genome may comprise two filler sequences, the first filler sequence may be located between the payload region and enhancer region, and the second filler sequence may be located between the intron region and exon region. In one embodiment, a viral genome may comprise two filler sequences, the first filler sequence may be located between the payload region and enhancer region, and the second filler sequence may be located between the intron region and 3′ ITR. In one embodiment, a viral genome may comprise two filler sequences, the first filler sequence may be located between the payload region and enhancer region, and the second filler sequence may be located between the enhancer region and polyadenylation signal sequence region. In one embodiment, a viral genome may comprise two filler sequences, the first filler sequence may be located between the payload region and enhancer region, and the second filler sequence may be located between the enhancer region and MCS region. In one embodiment, a viral genome may comprise two filler sequences, the first filler sequence may be located between the payload region and enhancer region, and the second filler sequence may be located between the enhancer region and exon region. In one embodiment, a viral genome may comprise two filler sequences, the first filler sequence may be located between the payload region and enhancer region, and the second filler sequence may be located between the enhancer region and 3′ ITR. In one embodiment, a viral genome may comprise two filler sequences, the first filler sequence may be located between the payload region and enhancer region, and the second filler sequence may be located between the polyadenylation signal sequence region and MCS region. In one embodiment, a viral genome may comprise two filler sequences, the first filler sequence may be located between the payload region and enhancer region, and the second filler sequence may be located between the polyadenylation signal sequence region and exon region. In one embodiment, a viral genome may comprise two filler sequences, the first filler sequence may be located between the payload region and enhancer region, and the second filler sequence may be located between the polyadenylation signal sequence region and 3′ ITR. In one embodiment, a viral genome may comprise two filler sequences, the first filler sequence may be located between the payload region and enhancer region, and the second filler sequence may be located between the MCS region and exon region. In one embodiment, a viral genome may comprise two filler sequences, the first filler sequence may be located between the payload region and enhancer region, and the second filler sequence may be located between the MCS region and 3′ ITR. In one embodiment, a viral genome may comprise two filler sequences, the first filler sequence may be located between the payload region and enhancer region, and the second filler sequence may be located between the exon region and 3′ ITR.

In one embodiment, a viral genome may comprise two filler sequences, the first filler sequence may be located between the payload region and polyadenylation signal sequence region, and the second filler sequence may be located between the intron region and enhancer region. In one embodiment, a viral genome may comprise two filler sequences, the first filler sequence may be located between the payload region and polyadenylation signal sequence region, and the second filler sequence may be located between the intron region and polyadenylation signal sequence region. In one embodiment, a viral genome may comprise two filler sequences, the first filler sequence may be located between the payload region and polyadenylation signal sequence region, and the second filler sequence may be located between the intron region and MCS region. In one embodiment, a viral genome may comprise two filler sequences, the first filler sequence may be located between the payload region and polyadenylation signal sequence region, and the second filler sequence may be located between the intron region and exon region. In one embodiment, a viral genome may comprise two filler sequences, the first filler sequence may be located between the payload region and polyadenylation signal sequence region, and the second filler sequence may be located between the intron region and 3′ ITR. In one embodiment, a viral genome may comprise two filler sequences, the first filler sequence may be located between the payload region and polyadenylation signal sequence region, and the second filler sequence may be located between the enhancer region and polyadenylation signal sequence region. In one embodiment, a viral genome may comprise two filler sequences, the first filler sequence may be located between the payload region and polyadenylation signal sequence region, and the second filler sequence may be located between the enhancer region and MCS region. In one embodiment, a viral genome may comprise two filler sequences, the first filler sequence may be located between the payload region and polyadenylation signal sequence region, and the second filler sequence may be located between the enhancer region and exon region. In one embodiment, a viral genome may comprise two filler sequences, the first filler sequence may be located between the payload region and polyadenylation signal sequence region, and the second filler sequence may be located between the enhancer region and 3′ ITR. In one embodiment, a viral genome may comprise two filler sequences, the first filler sequence may be located between the payload region and polyadenylation signal sequence region, and the second filler sequence may be located between the polyadenylation signal sequence region and MCS region. In one embodiment, a viral genome may comprise two filler sequences, the first filler sequence may be located between the payload region and polyadenylation signal sequence region, and the second filler sequence may be located between the polyadenylation signal sequence region and exon region. In one embodiment, a viral genome may comprise two filler sequences, the first filler sequence may be located between the payload region and polyadenylation signal sequence region, and the second filler sequence may be located between the polyadenylation signal sequence region and 3′ ITR. In one embodiment, a viral genome may comprise two filler sequences, the first filler sequence may be located between the payload region and polyadenylation signal sequence region, and the second filler sequence may be located between the MCS region and exon region. In one embodiment, a viral genome may comprise two filler sequences, the first filler sequence may be located between the payload region and polyadenylation signal sequence region, and the second filler sequence may be located between the MCS region and 3′ ITR. In one embodiment, a viral genome may comprise two filler sequences, the first filler sequence may be located between the payload region and polyadenylation signal sequence region, and the second filler sequence may be located between the exon region and 3′ ITR.

In one embodiment, a viral genome may comprise two filler sequences, the first filler sequence may be located between the payload region and MCS region, and the second filler sequence may be located between the intron region and enhancer region. In one embodiment, a viral genome may comprise two filler sequences, the first filler sequence may be located between the payload region and MCS region, and the second filler sequence may be located between the intron region and polyadenylation signal sequence region. In one embodiment, a viral genome may comprise two filler sequences, the first filler sequence may be located between the payload region and MCS region, and the second filler sequence may be located between the intron region and MCS region. In one embodiment, a viral genome may comprise two filler sequences, the first filler sequence may be located between the payload region and MCS region, and the second filler sequence may be located between the intron region and exon region. In one embodiment, a viral genome may comprise two filler sequences, the first filler sequence may be located between the payload region and MCS region, and the second filler sequence may be located between the intron region and 3′ ITR. In one embodiment, a viral genome may comprise two filler sequences, the first filler sequence may be located between the payload region and MCS region, and the second filler sequence may be located between the enhancer region and polyadenylation signal sequence region. In one embodiment, a viral genome may comprise two filler sequences, the first filler sequence may be located between the payload region and MCS region, and the second filler sequence may be located between the enhancer region and MCS region. In one embodiment, a viral genome may comprise two filler sequences, the first filler sequence may be located between the payload region and MCS region, and the second filler sequence may be located between the enhancer region and exon region. In one embodiment, a viral genome may comprise two filler sequences, the first filler sequence may be located between the payload region and MCS region, and the second filler sequence may be located between the enhancer region and 3′ ITR. In one embodiment, a viral genome may comprise two filler sequences, the first filler sequence may be located between the payload region and MCS region, and the second filler sequence may be located between the polyadenylation signal sequence region and MCS region. In one embodiment, a viral genome may comprise two filler sequences, the first filler sequence may be located between the payload region and MCS region, and the second filler sequence may be located between the polyadenylation signal sequence region and exon region. In one embodiment, a viral genome may comprise two filler sequences, the first filler sequence may be located between the payload region and MCS region, and the second filler sequence may be located between the polyadenylation signal sequence region and 3′ ITR. In one embodiment, a viral genome may comprise two filler sequences, the first filler sequence may be located between the payload region and MCS region, and the second filler sequence may be located between the MCS region and exon region. In one embodiment, a viral genome may comprise two filler sequences, the first filler sequence may be located between the payload region and MCS region, and the second filler sequence may be located between the MCS region and 3′ ITR. In one embodiment, a viral genome may comprise two filler sequences, the first filler sequence may be located between the payload region and MCS region, and the second filler sequence may be located between the exon region and 3′ ITR.

In one embodiment, a viral genome may comprise two filler sequences, the first filler sequence may be located between the payload region and exon region, and the second filler sequence may be located between the intron region and enhancer region. In one embodiment, a viral genome may comprise two filler sequences, the first filler sequence may be located between the payload region and exon region, and the second filler sequence may be located between the intron region and polyadenylation signal sequence region. In one embodiment, a viral genome may comprise two filler sequences, the first filler sequence may be located between the payload region and exon region, and the second filler sequence may be located between the intron region and MCS region. In one embodiment, a viral genome may comprise two filler sequences, the first filler sequence may be located between the payload region and exon region, and the second filler sequence may be located between the intron region and exon region. In one embodiment, a viral genome may comprise two filler sequences, the first filler sequence may be located between the payload region and exon region, and the second filler sequence may be located between the intron region and 3′ ITR. In one embodiment, a viral genome may comprise two filler sequences, the first filler sequence may be located between the payload region and exon region, and the second filler sequence may be located between the enhancer region and polyadenylation signal sequence region. In one embodiment, a viral genome may comprise two filler sequences, the first filler sequence may be located between the payload region and exon region, and the second filler sequence may be located between the enhancer region and MCS region. In one embodiment, a viral genome may comprise two filler sequences, the first filler sequence may be located between the payload region and exon region, and the second filler sequence may be located between the enhancer region and exon region. In one embodiment, a viral genome may comprise two filler sequences, the first filler sequence may be located between the payload region and exon region, and the second filler sequence may be located between the enhancer region and 3′ ITR. In one embodiment, a viral genome may comprise two filler sequences, the first filler sequence may be located between the payload region and exon region, and the second filler sequence may be located between the polyadenylation signal sequence region and MCS region. In one embodiment, a viral genome may comprise two filler sequences, the first filler sequence may be located between the payload region and exon region, and the second filler sequence may be located between the polyadenylation signal sequence region and exon region. In one embodiment, a viral genome may comprise two filler sequences, the first filler sequence may be located between the payload region and exon region, and the second filler sequence may be located between the polyadenylation signal sequence region and 3′ ITR. In one embodiment, a viral genome may comprise two filler sequences, the first filler sequence may be located between the payload region and exon region, and the second filler sequence may be located between the MCS region and exon region. In one embodiment, a viral genome may comprise two filler sequences, the first filler sequence may be located between the payload region and exon region, and the second filler sequence may be located between the MCS region and 3′ ITR. In one embodiment, a viral genome may comprise two filler sequences, the first filler sequence may be located between the payload region and exon region, and the second filler sequence may be located between the exon region and 3′ ITR.

In one embodiment, a viral genome may comprise two filler sequences, the first filler sequence may be located between the payload region and 3′ ITR region, and the second filler sequence may be located between the intron region and enhancer region. In one embodiment, a viral genome may comprise two filler sequences, the first filler sequence may be located between the payload region and 3′ ITR region, and the second filler sequence may be located between the intron region and polyadenylation signal sequence region. In one embodiment, a viral genome may comprise two filler sequences, the first filler sequence may be located between the payload region and 3′ ITR region, and the second filler sequence may be located between the intron region and MCS region. In one embodiment, a viral genome may comprise two filler sequences, the first filler sequence may be located between the payload region and 3′ ITR region, and the second filler sequence may be located between the intron region and exon region. In one embodiment, a viral genome may comprise two filler sequences, the first filler sequence may be located between the payload region and 3′ ITR region, and the second filler sequence may be located between the intron region and 3′ ITR. In one embodiment, a viral genome may comprise two filler sequences, the first filler sequence may be located between the payload region and 3′ ITR region, and the second filler sequence may be located between the enhancer region and polyadenylation signal sequence region. In one embodiment, a viral genome may comprise two filler sequences, the first filler sequence may be located between the payload region and 3′ ITR region, and the second filler sequence may be located between the enhancer region and MCS region. In one embodiment, a viral genome may comprise two filler sequences, the first filler sequence may be located between the payload region and 3′ ITR region, and the second filler sequence may be located between the enhancer region and exon region. In one embodiment, a viral genome may comprise two filler sequences, the first filler sequence may be located between the payload region and 3′ ITR region, and the second filler sequence may be located between the enhancer region and 3′ ITR. In one embodiment, a viral genome may comprise two filler sequences, the first filler sequence may be located between the payload region and 3′ ITR region, and the second filler sequence may be located between the polyadenylation signal sequence region and MCS region. In one embodiment, a viral genome may comprise two filler sequences, the first filler sequence may be located between the payload region and 3′ ITR region, and the second filler sequence may be located between the polyadenylation signal sequence region and exon region. In one embodiment, a viral genome may comprise two filler sequences, the first filler sequence may be located between the payload region and 3′ ITR region, and the second filler sequence may be located between the polyadenylation signal sequence region and 3′ ITR. In one embodiment, a viral genome may comprise two filler sequences, the first filler sequence may be located between the payload region and 3′ ITR region, and the second filler sequence may be located between the MCS region and exon region. In one embodiment, a viral genome may comprise two filler sequences, the first filler sequence may be located between the payload region and 3′ ITR region, and the second filler sequence may be located between the MCS region and 3′ ITR. In one embodiment, a viral genome may comprise two filler sequences, the first filler sequence may be located between the payload region and 3′ ITR region, and the second filler sequence may be located between the exon region and 3′ ITR.

In one embodiment, a viral genome may comprise two filler sequences, the first filler sequence may be located between the intron region and enhancer region, and the second filler sequence may be located between the enhancer region and polyadenylation signal sequence region. In one embodiment, a viral genome may comprise two filler sequences, the first filler sequence may be located between the intron region and enhancer region, and the second filler sequence may be located between the enhancer region and MCS region. In one embodiment, a viral genome may comprise two filler sequences, the first filler sequence may be located between the intron region and enhancer region, and the second filler sequence may be located between the enhancer region and exon region. In one embodiment, a viral genome may comprise two filler sequences, the first filler sequence may be located between the intron region and enhancer region, and the second filler sequence may be located between the enhancer region and 3′ ITR. In one embodiment, a viral genome may comprise two filler sequences, the first filler sequence may be located between the intron region and enhancer region, and the second filler sequence may be located between the polyadenylation signal sequence region and MCS region. In one embodiment, a viral genome may comprise two filler sequences, the first filler sequence may be located between the intron region and enhancer region, and the second filler sequence may be located between the polyadenylation signal sequence region and exon region. In one embodiment, a viral genome may comprise two filler sequences, the first filler sequence may be located between the intron region and enhancer region, and the second filler sequence may be located between the polyadenylation signal sequence region and 3′ ITR. In one embodiment, a viral genome may comprise two filler sequences, the first filler sequence may be located between the intron region and enhancer region, and the second filler sequence may be located between the MCS region and exon region. In one embodiment, a viral genome may comprise two filler sequences, the first filler sequence may be located between the intron region and enhancer region, and the second filler sequence may be located between the MCS region and 3′ ITR. In one embodiment, a viral genome may comprise two filler sequences, the first filler sequence may be located between the intron region and enhancer region, and the second filler sequence may be located between the exon region and 3′ ITR.

In one embodiment, a viral genome may comprise two filler sequences, the first filler sequence may be located between the intron region and polyadenylation signal sequence region, and the second filler sequence may be located between the enhancer region and polyadenylation signal sequence region. In one embodiment, a viral genome may comprise two filler sequences, the first filler sequence may be located between the intron region and polyadenylation signal sequence region, and the second filler sequence may be located between the enhancer region and MCS region. In one embodiment, a viral genome may comprise two filler sequences, the first filler sequence may be located between the intron region and polyadenylation signal sequence region, and the second filler sequence may be located between the enhancer region and exon region. In one embodiment, a viral genome may comprise two filler sequences, the first filler sequence may be located between the intron region and polyadenylation signal sequence region, and the second filler sequence may be located between the enhancer region and 3′ ITR. In one embodiment, a viral genome may comprise two filler sequences, the first filler sequence may be located between the intron region and polyadenylation signal sequence region, and the second filler sequence may be located between the polyadenylation signal sequence region and MCS region. In one embodiment, a viral genome may comprise two filler sequences, the first filler sequence may be located between the intron region and polyadenylation signal sequence region, and the second filler sequence may be located between the polyadenylation signal sequence region and exon region. In one embodiment, a viral genome may comprise two filler sequences, the first filler sequence may be located between the intron region and polyadenylation signal sequence region, and the second filler sequence may be located between the polyadenylation signal sequence region and 3′ ITR. In one embodiment, a viral genome may comprise two filler sequences, the first filler sequence may be located between the intron region and polyadenylation signal sequence region, and the second filler sequence may be located between the MCS region and exon region. In one embodiment, a viral genome may comprise two filler sequences, the first filler sequence may be located between the intron region and polyadenylation signal sequence region, and the second filler sequence may be located between the MCS region and 3′ ITR. In one embodiment, a viral genome may comprise two filler sequences, the first filler sequence may be located between the intron region and polyadenylation signal sequence region, and the second filler sequence may be located between the exon region and 3′ ITR.

In one embodiment, a viral genome may comprise two filler sequences, the first filler sequence may be located between the intron region and MCS region, and the second filler sequence may be located between the enhancer region and polyadenylation signal sequence region. In one embodiment, a viral genome may comprise two filler sequences, the first filler sequence may be located between the intron region and MCS region, and the second filler sequence may be located between the enhancer region and MCS region. In one embodiment, a viral genome may comprise two filler sequences, the first filler sequence may be located between the intron region and MCS region, and the second filler sequence may be located between the enhancer region and exon region. In one embodiment, a viral genome may comprise two filler sequences, the first filler sequence may be located between the intron region and MCS region, and the second filler sequence may be located between the enhancer region and 3′ ITR. In one embodiment, a viral genome may comprise two filler sequences, the first filler sequence may be located between the intron region and MCS region, and the second filler sequence may be located between the polyadenylation signal sequence region and MCS region. In one embodiment, a viral genome may comprise two filler sequences, the first filler sequence may be located between the intron region and MCS region, and the second filler sequence may be located between the polyadenylation signal sequence region and exon region. In one embodiment, a viral genome may comprise two filler sequences, the first filler sequence may be located between the intron region and MCS region, and the second filler sequence may be located between the polyadenylation signal sequence region and 3′ ITR. In one embodiment, a viral genome may comprise two filler sequences, the first filler sequence may be located between the intron region and MCS region, and the second filler sequence may be located between the MCS region and exon region. In one embodiment, a viral genome may comprise two filler sequences, the first filler sequence may be located between the intron region and MCS region, and the second filler sequence may be located between the MCS region and 3′ ITR. In one embodiment, a viral genome may comprise two filler sequences, the first filler sequence may be located between the intron region and MCS region, and the second filler sequence may be located between the exon region and 3′ ITR.

In one embodiment, a viral genome may comprise two filler sequences, the first filler sequence may be located between the intron region and exon region, and the second filler sequence may be located between the enhancer region and polyadenylation signal sequence region. In one embodiment, a viral genome may comprise two filler sequences, the first filler sequence may be located between the intron region and exon region, and the second filler sequence may be located between the enhancer region and MCS region. In one embodiment, a viral genome may comprise two filler sequences, the first filler sequence may be located between the intron region and exon region, and the second filler sequence may be located between the enhancer region and exon region. In one embodiment, a viral genome may comprise two filler sequences, the first filler sequence may be located between the intron region and exon region, and the second filler sequence may be located between the enhancer region and 3′ ITR. In one embodiment, a viral genome may comprise two filler sequences, the first filler sequence may be located between the intron region and exon region, and the second filler sequence may be located between the polyadenylation signal sequence region and MCS region. In one embodiment, a viral genome may comprise two filler sequences, the first filler sequence may be located between the intron region and exon region, and the second filler sequence may be located between the polyadenylation signal sequence region and exon region. In one embodiment, a viral genome may comprise two filler sequences, the first filler sequence may be located between the intron region and exon region, and the second filler sequence may be located between the polyadenylation signal sequence region and 3′ ITR. In one embodiment, a viral genome may comprise two filler sequences, the first filler sequence may be located between the intron region and exon region, and the second filler sequence may be located between the MCS region and exon region. In one embodiment, a viral genome may comprise two filler sequences, the first filler sequence may be located between the intron region and exon region, and the second filler sequence may be located between the MCS region and 3′ ITR. In one embodiment, a viral genome may comprise two filler sequences, the first filler sequence may be located between the intron region and exon region, and the second filler sequence may be located between the exon region and 3′ ITR.

In one embodiment, a viral genome may comprise two filler sequences, the first filler sequence may be located between the intron region and 3′ITR, and the second filler sequence may be located between the enhancer region and polyadenylation signal sequence region. In one embodiment, a viral genome may comprise two filler sequences, the first filler sequence may be located between the intron region and 3′ITR, and the second filler sequence may be located between the enhancer region and MCS region. In one embodiment, a viral genome may comprise two filler sequences, the first filler sequence may be located between the intron region and 3′ITR, and the second filler sequence may be located between the enhancer region and exon region. In one embodiment, a viral genome may comprise two filler sequences, the first filler sequence may be located between the intron region and 3′ITR, and the second filler sequence may be located between the enhancer region and 3′ ITR. In one embodiment, a viral genome may comprise two filler sequences, the first filler sequence may be located between the intron region and 3′ITR, and the second filler sequence may be located between the polyadenylation signal sequence region and MCS region. In one embodiment, a viral genome may comprise two filler sequences, the first filler sequence may be located between the intron region and 3′ITR, and the second filler sequence may be located between the polyadenylation signal sequence region and exon region. In one embodiment, a viral genome may comprise two filler sequences, the first filler sequence may be located between the intron region and 3′ITR, and the second filler sequence may be located between the polyadenylation signal sequence region and 3′ ITR. In one embodiment, a viral genome may comprise two filler sequences, the first filler sequence may be located between the intron region and 3′ITR, and the second filler sequence may be located between the MCS region and exon region. In one embodiment, a viral genome may comprise two filler sequences, the first filler sequence may be located between the intron region and 3′ITR, and the second filler sequence may be located between the MCS region and 3′ ITR. In one embodiment, a viral genome may comprise two filler sequences, the first filler sequence may be located between the intron region and 3′ITR, and the second filler sequence may be located between the exon region and 3′ ITR.

In one embodiment, a viral genome may comprise two filler sequences, the first filler sequence may be located between the enhancer region and polyadenylation signal sequence region, and the second filler sequence may be located between the polyadenylation signal sequence region and MCS region. In one embodiment, a viral genome may comprise two filler sequences, the first filler sequence may be located between the enhancer region and polyadenylation signal sequence region, and the second filler sequence may be located between the polyadenylation signal sequence region and exon region. In one embodiment, a viral genome may comprise two filler sequences, the first filler sequence may be located between the enhancer region and polyadenylation signal sequence region, and the second filler sequence may be located between the polyadenylation signal sequence region and 3′ ITR. In one embodiment, a viral genome may comprise two filler sequences, the first filler sequence may be located between the enhancer region and polyadenylation signal sequence region, and the second filler sequence may be located between the MCS region and exon region. In one embodiment, a viral genome may comprise two filler sequences, the first filler sequence may be located between the enhancer region and polyadenylation signal sequence region, and the second filler sequence may be located between the MCS region and 3′ ITR. In one embodiment, a viral genome may comprise two filler sequences, the first filler sequence may be located between the enhancer region and polyadenylation signal sequence region, and the second filler sequence may be located between the exon region and 3′ ITR.

In one embodiment, a viral genome may comprise two filler sequences, the first filler sequence may be located between the enhancer region and MCS region, and the second filler sequence may be located between the polyadenylation signal sequence region and MCS region. In one embodiment, a viral genome may comprise two filler sequences, the first filler sequence may be located between the enhancer region and MCS region, and the second filler sequence may be located between the polyadenylation signal sequence region and exon region. In one embodiment, a viral genome may comprise two filler sequences, the first filler sequence may be located between the enhancer region and MCS region, and the second filler sequence may be located between the polyadenylation signal sequence region and 3′ ITR. In one embodiment, a viral genome may comprise two filler sequences, the first filler sequence may be located between the enhancer region and MCS region, and the second filler sequence may be located between the MCS region and exon region. In one embodiment, a viral genome may comprise two filler sequences, the first filler sequence may be located between the enhancer region and MCS region, and the second filler sequence may be located between the MCS region and 3′ ITR. In one embodiment, a viral genome may comprise two filler sequences, the first filler sequence may be located between the enhancer region and MCS region, and the second filler sequence may be located between the exon region and 3′ ITR.

In one embodiment, a viral genome may comprise two filler sequences, the first filler sequence may be located between the enhancer region and exon region, and the second filler sequence may be located between the polyadenylation signal sequence region and MCS region. In one embodiment, a viral genome may comprise two filler sequences, the first filler sequence may be located between the enhancer region and exon region, and the second filler sequence may be located between the polyadenylation signal sequence region and exon region. In one embodiment, a viral genome may comprise two filler sequences, the first filler sequence may be located between the enhancer region and exon region, and the second filler sequence may be located between the polyadenylation signal sequence region and 3′ ITR. In one embodiment, a viral genome may comprise two filler sequences, the first filler sequence may be located between the enhancer region and exon region, and the second filler sequence may be located between the MCS region and exon region. In one embodiment, a viral genome may comprise two filler sequences, the first filler sequence may be located between the enhancer region and exon region, and the second filler sequence may be located between the MCS region and 3′ ITR. In one embodiment, a viral genome may comprise two filler sequences, the first filler sequence may be located between the enhancer region and exon region, and the second filler sequence may be located between the exon region and 3′ ITR.

In one embodiment, a viral genome may comprise two filler sequences, the first filler sequence may be located between the enhancer region and 3′ ITR, and the second filler sequence may be located between the polyadenylation signal sequence region and MCS region. In one embodiment, a viral genome may comprise two filler sequences, the first filler sequence may be located between the enhancer region and 3′ ITR, and the second filler sequence may be located between the polyadenylation signal sequence region and exon region. In one embodiment, a viral genome may comprise two filler sequences, the first filler sequence may be located between the enhancer region and 3′ ITR, and the second filler sequence may be located between the polyadenylation signal sequence region and 3′ ITR. In one embodiment, a viral genome may comprise two filler sequences, the first filler sequence may be located between the enhancer region and 3′ ITR, and the second filler sequence may be located between the MCS region and exon region. In one embodiment, a viral genome may comprise two filler sequences, the first filler sequence may be located between the enhancer region and 3′ ITR, and the second filler sequence may be located between the MCS region and 3′ ITR. In one embodiment, a viral genome may comprise two filler sequences, the first filler sequence may be located between the enhancer region and 3′ ITR, and the second filler sequence may be located between the exon region and 3′ ITR.

In one embodiment, a viral genome may comprise two filler sequences, the first filler sequence may be located between the polyadenylation signal sequence region and MCS region, and the second filler sequence may be located between the MCS region and exon region. In one embodiment, a viral genome may comprise two filler sequences, the first filler sequence may be located between the polyadenylation signal sequence region and MCS region, and the second filler sequence may be located between the MCS region and 3′ ITR. In one embodiment, a viral genome may comprise two filler sequences, the first filler sequence may be located between the polyadenylation signal sequence region and MCS region, and the second filler sequence may be located between the exon region and 3′ ITR.

In one embodiment, a viral genome may comprise two filler sequences, the first filler sequence may be located between the polyadenylation signal sequence region and exon region, and the second filler sequence may be located between the MCS region and exon region. In one embodiment, a viral genome may comprise two filler sequences, the first filler sequence may be located between the polyadenylation signal sequence region and exon region, and the second filler sequence may be located between the MCS region and 3′ ITR. In one embodiment, a viral genome may comprise two filler sequences, the first filler sequence may be located between the polyadenylation signal sequence region and exon region, and the second filler sequence may be located between the exon region and 3′ ITR.

In one embodiment, a viral genome may comprise two filler sequences, the first filler sequence may be located between the polyadenylation signal sequence region and 3′ ITR, and the second filler sequence may be located between the MCS region and exon region. In one embodiment, a viral genome may comprise two filler sequences, the first filler sequence may be located between the polyadenylation signal sequence region and 3′ ITR, and the second filler sequence may be located between the MCS region and 3′ ITR. In one embodiment, a viral genome may comprise two filler sequences, the first filler sequence may be located between the polyadenylation signal sequence region and 3′ ITR, and the second filler sequence may be located between the exon region and 3′ ITR.

In one embodiment, a viral genome may comprise two filler sequences, the first filler sequence may be located between the MCS region and exon region, and the second filler sequence may be located between the exon region and 3′ ITR.

### Payloads of the Invention

The AAV particles of the present disclosure comprise at least one payload region. As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid. Payloads of the present invention typically encode modulatory polynucleotides or fragments or variants thereof.

The payload region may be constructed in such a way as to reflect a region similar to or mirroring the natural organization of an mRNA.

The payload region may comprise a combination of coding and non-coding nucleic acid sequences.

In some embodiments, the AAV payload region may encode a coding or non-coding RNA.

In one embodiment, the AAV particle comprises a viral genome with a payload region comprising nucleic acid sequences encoding a siRNA, miRNA or other RNAi agent. In such an embodiment, a viral genome encoding more than one polypeptide may be replicated and packaged into a viral particle. A target cell transduced with a viral particle may express the encoded siRNA, miRNA or other RNAi agent inside a single cell.

### Modulatory Polynucleotides

In one embodiment, modulatory polynucleotides, e.g., RNA or DNA molecules, may be used to treat at least one neurodegenerative disease. As used herein, a “modulatory polynucleotide” is any nucleic acid sequence(s) which functions to modulate (either increase or decrease) the level or amount of a target gene, e.g., mRNA or protein levels.

In one embodiment, the modulatory polynucleotides may comprise at least one nucleic acid sequence encoding at least one siRNA molecule. The nucleic acids may, independently if there is more than one, encode 1, 2, 3, 4, 5, 6, 7, 8, 9, or more than 9 siRNA molecules.

In one embodiment, the molecular scaffold may be located downstream of a CMV promoter, fragment or variant thereof.

In one embodiment, the molecular scaffold may be located downstream of a CBA promoter, fragment or variant thereof.

In one embodiment, the molecular scaffold may be a natural pri-miRNA scaffold located downstream of a CMV promoter. As a non-limiting example, the natural pri-miRNA scaffold is derived from the human miR155 scaffold.

In one embodiment, the molecular scaffold may be a natural pri-miRNA scaffold located downstream of a CBA promoter.

In one embodiment, the selection of a molecular scaffold and modulatory polynucleotide is determined by a method of comparing modulatory polynucleotides in pri-miRNA (see e.g., the method described by Miniarikova et al. Design, Characterization, and Lead Selection of Therapeutic miRNAs Targeting Huntingtin for Development of Gene Therapy for Huntington's Disease. Molecular Therapy-Nucleic Acids (2016) 5, e297 and International Publication No. WO2016102664; the contents of each of which are herein incorporated by reference in their entireties). To evaluate the activities of the modulatory polynucleotides, the molecular scaffold used which may be used is a human pri-miRNA scaffold (e.g., miR155 scaffold) and the promoter may be CMV. The activity may be determined in vitro using HEK293T cells and a reporter (e.g., Luciferase).

In order to evaluate the optimal molecular scaffold for the modulatory polynucleotide, the modulatory polynucleotide is used in pri-miRNA scaffolds with a CAG promoter. The constructs are co-transfected with a reporter (e.g., luciferase reporter) at 50 ng. Constructs with greater than 80% knockdown at 50 ng co-transfection are considered efficient. In one aspect, the constructs with strong guide-strand activity are preferred. The molecular scaffolds can be processed in HEK293T cells by NGS to determine guide-passenger ratios, and processing variability.

In one embodiment, the disease to be treated is HD and the modulatory polynucleotide may, but it not limited to, targeting exon 1, CAG repeats, SNP rs362331 in exon 50 and/or SNP rs362307 in exon 67. For exon 1 targeting, the modulatory polynucleotide is determined to be efficient at HTT knockdown if the knockdown is 80% or greater. For CAG targeting, the modulatory polynucleotide is determined to be efficient at HTT knockdown if the knockdown is at least 60%. For SNP targeting, the modulatory polynucleotide is determined to be efficient at HTT knockdown if the knockdown is at least 60%. For allele selectivity for CAG repeats or SNP targeting the modulatory polynucleotides may comprise at least 1 substitution in order to improve allele selectivity. As a non-limiting example, substitution may be a G or C replaced with a T or corresponding U and A or T/U replaced by a C.

To evaluate the molecular scaffolds and modulatory polynucleotides in vivo the molecular scaffolds comprising the modulatory polynucleotides are packaged in AAV (e.g., the serotype may be AAV5 (see e.g., the method and constructs described in WO2015060722, the contents of which are herein incorporated by reference in their entirety)) and administered to an in vivo model (e.g., For HD, a Hu128/21 HD mouse may be used) and the guide-passenger ratios, 5′ and 3′ end processing, reversal of guide and passenger strands, and knockdown can be determined in different areas of the model.

In one embodiment, the selection of a molecular scaffold and modulatory polynucleotide is determined by a method of comparing modulatory polynucleotides in natural pri-miRNA and synthetic pri-miRNA. The modulatory polynucleotide may, but it not limited to, targeting an exon other than exon 1. To evaluate the activities of the modulatory polynucleotides, the molecular scaffold is used with a CBA promoter. In one aspect, the activity may be determined in vitro using HEK293T cells, HeLa cell and a reporter (e.g., Luciferase) and knockdown efficient modulatory polynucleotides showed the gene of interest knockdown of at least 80% in the cell tested. Additionally, the modulatory polynucleotides which are considered most efficient showed low to no significant passenger strand (p-strand) activity. In another aspect, the endogenous gene of interest knockdown efficacy is evaluated by transfection in vitro using HEK293T cells, HeLa cell and a reporter. Efficient modulatory polynucleotides show greater than 50% endogenous gene of interest knockdown. In yet another aspect, the endogenous gene of interest knockdown efficacy is evaluated in different cell types (e.g., HEK293, HeLa, primary astrocytes, U251 astrocytes, SH-SY5Y neuron cells and fibroblasts from subjects with the disease to be treated) by infection (e.g., AAV2). Efficient modulatory polynucleotides show greater than 60% endogenous gene of interest knockdown.

To evaluate the molecular scaffolds and modulatory polynucleotides in vivo the molecular scaffolds comprising the modulatory polynucleotides are packaged in AAV and administered to an in vivo model (e.g., For treating HD, a YAC128 HD mouse model may be used) and the guide-passenger ratios, 5′ and 3′ end processing, ratio of guide to passenger strands, and knockdown can be determined in different areas of the model (e.g., tissue regions). The molecular scaffolds can be processed from in vivo samples by NGS to determine guide-passenger ratios, and processing variability.

In one embodiment, the modulatory polynucleotide is designed using at least one of the following properties: loop variant, seed mismatch/bulge/wobble variant, stem mismatch, loop variant and vassal stem mismatch variant, seed mismatch and basal stem mismatch variant, stem mismatch and basal stem mismatch variant, seed wobble and basal stem wobble variant, or a stem sequence variant.

siRNA Molecules

The present invention relates to RNA interference (RNAi) induced inhibition of gene expression for treating neurodegenerative disorders. Provided herein are siRNA duplexes or encoded dsRNA that target the gene of interest (referred to herein collectively as “siRNA molecules”). Such siRNA duplexes or encoded dsRNA can reduce or silence gene expression in cells, such as but not limited to, medium spiny neurons, cortical neurons and/or astrocytes.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2 nucleotide 3′ overhangs and that match the nucleic acid sequence of the target gene. These short RNA species may be naturally produced in vivo by Dicer-mediated cleavage of larger dsRNAs and they are functional in mammalian cells.

Naturally expressed small RNA molecules, named microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′-UTR of the target mRNAs. A single miRNA may target more than 100 transcripts from various genes, and one mRNA may be targeted by different miRNAs.

siRNA duplexes or dsRNA targeting a specific mRNA may be designed and synthesized in vitro and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

RNAi molecules which were designed to target against a nucleic acid sequence that encodes poly-glutamine repeat proteins which cause poly-glutamine expansion diseases such as Huntington's Disease, are described in U.S. Pat. Nos. 9,169,483 and 9,181,544 and International Patent Publication No. WO2015179525, the content of each of which is herein incorporated by reference in their entirety. U.S. Pat. Nos. 9,169,483 and 9,181,544 and International Patent Publication No. WO2015179525 each provide isolated RNA duplexes comprising a first strand of RNA (e.g., 15 contiguous nucleotides) and second strand of RNA (e.g., complementary to at least 12 contiguous nucleotides of the first strand) where the RNA duplex is about 15 to 30 base pairs in length. The first strand of RNA and second strand of RNA may be operably linked by an RNA loop (˜4 to 50 nucleotides) to form a hairpin structure which may be inserted into an expression cassette. Non-limiting examples of loop portions include SEQ ID NO: 9-14 of U.S. Pat. No. 9,169,483, the content of which is herein incorporated by reference in its entirety. Non-limiting examples of strands of RNA which may be used, either full sequence or part of the sequence, to form RNA duplexes include SEQ ID NO: 1-8 of U.S. Pat. No. 9,169,483 and SEQ ID NO: 1-11, 33-59, 208-210, 213-215 and 218-221 of U.S. Pat. No. 9,181,544, the contents of each of which is herein incorporated by reference in its entirety. Non-limiting examples of RNAi molecules include SEQ ID NOs: 1-8 of U.S. Pat. No. 9,169,483, SEQ ID NOs: 1-11, 33-59, 208-210, 213-215 and 218-221 of U.S. Pat. No. 9,181,544 and SEQ ID NOs: 1, 6, 7, and 35-38 of International Patent Publication No. WO2015179525, the contents of each of which is herein incorporated by reference in their entirety.

In vitro synthetized siRNA molecules may be introduced into cells in order to activate RNAi. An exogenous siRNA duplex, when it is introduced into cells, similar to the endogenous dsRNAs, can be assembled to form the RNA Induced Silencing Complex (RISC), a multiunit complex that interacts with RNA sequences that are complementary to one of the two strands of the siRNA duplex (i.e., the antisense strand). During the process, the sense strand (or passenger strand) of the siRNA is lost from the complex, while the antisense strand (or guide strand) of the siRNA is matched with its complementary RNA. In particular, the targets of siRNA containing RISC complexes are mRNAs presenting a perfect sequence complementarity. Then, siRNA mediated gene silencing occurs by cleaving, releasing and degrading the target.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases, it requires higher concentration of the ss-siRNA to achieve the effective gene silencing potency of the corresponding duplex.

Any of the foregoing molecules may be encoded by a viral genome.

Design and Sequences of siRNA Duplexes Targeting Gene of Interest

The present invention provides small interfering RNA (siRNA) duplexes (and modulatory polynucleotides encoding them) that target mRNA to interfere with gene expression and/or protein production.

The encoded siRNA duplex of the present invention contains an antisense strand and a sense strand hybridized together forming a duplex structure, wherein the antisense strand is complementary to the nucleic acid sequence of the targeted gene, and wherein the sense strand is homologous to the nucleic acid sequence of the targeted gene. In some aspects, the 5′ end of the antisense strand has a 5′ phosphate group and the 3′ end of the sense strand contains a 3′hydroxyl group. In other aspects, there are none, one or 2 nucleotide overhangs at the 3′end of each strand.

Some guidelines for designing siRNAs have been proposed in the art. These guidelines generally recommend generating a 19-nucleotide duplexed region, symmetric 2-3 nucleotide 3′ overhangs, 5′-phosphate and 3′-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such consideration, together with the specific sequence of a target gene, highly effective siRNA molecules essential for suppressing mammalian target gene expression may be readily designed.

According to the present invention, siRNA molecules (e.g., siRNA duplexes or encoded dsRNA) that target the gene of interest are designed. Such siRNA molecules can specifically, suppress gene expression and protein production. In some aspects, the siRNA molecules are designed and used to selectively “knock out” gene variants in cells, i.e., mutated transcripts. In some aspects, the siRNA molecules are designed and used to selectively “knock down” gene variants in cells. In other aspects, the siRNA molecules are able to inhibit or suppress both the wild type and mutated version of the gene of interest.

In one embodiment, an siRNA molecule of the present invention comprises a sense strand and a complementary antisense strand in which both strands are hybridized together to form a duplex structure. The antisense strand has sufficient complementarity to the target mRNA sequence to direct target-specific RNAi, i.e., the siRNA molecule has a sequence sufficient to trigger the destruction of the target mRNA by the RNAi machinery or process.

In one embodiment, an siRNA molecule of the present invention comprises a sense strand and a complementary antisense strand in which both strands are hybridized together to form a duplex structure and where the start site of the hybridization to the mRNA is between nucleotide 10 and 7000 on the mRNA sequence. As a non-limiting example, the start site may be between nucleotide 10-20, 20-30, 30-40, 40-50, 60-70, 70-80, 80-90, 90-100, 100-150, 150-200, 200-250, 250-300, 300-350, 350-400, 400-450, 450-500, 500-550, 550-600, 600-650, 650-700, 700-70, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, 3200-3250, 3250-3300, 3300-3350, 3350-3400, 3400-3450, 3450-3500, 3500-3550, 3550-3600, 3600-3650, 3650-3700, 3700-3750, 3750-3800, 3800-3850, 3850-3900, 3900-3950, 3950-4000, 4000-4050, 4050-4100, 4100-4150, 4150-4200, 4200-4250, 4250-4300, 4300-4350, 4350-4400, 4400-4450, 4450-4500, 4500-4550, 4550-4600, 4600-4650, 4650-4700, 4700-4750, 4750-4800, 4800-4850, 4850-4900, 4900-4950, 4950-5000, 5000-5050, 5050-5100, 5100-5150, 5150-5200, 5200-5250, 5250-5300, 5300-5350, 5350-5400, 5400-5450, 5450-5500, 5500-5550, 5550-5600, 5600-5650, 5650-5700, 5700-5750, 5750-5800, 5800-5850, 5850-5900, 5900-5950, 5950-6000, 6000-6050, 6050-6100, 6100-6150, 6150-6200, 6200-6250, 6250-6300, 6300-6350, 6350-6400, 6400-6450, 6450-6500, 6500-6550, 6550-6600, 6600-6650, 6650-6700, 6700-6750, 6750-6800, 6800-6850, 6850-6900, 6900-6950, 6950-7000, 7000-7050, 7050-7100, 7100-7150, 7150-7200, 7200-7250, 7250-7300, 7300-7350, 7350-7400, 7400-7450, 7450-7500, 7500-7550, 7550-7600, 7600-7650, 7650-7700, 7700-7750, 7750-7800, 7800-7850, 7850-7900, 7900-7950, 7950-8000, 8000-8050, 8050-8100, 8100-8150, 8150-8200, 8200-8250, 8250-8300, 8300-8350, 8350-8400, 8400-8450, 8450-8500, 8500-8550, 8550-8600, 8600-8650, 8650-8700, 8700-8750, 8750-8800, 8800-8850, 8850-8900, 8900-8950, 8950-9000, 9000-9050, 9050-9100, 9100-9150, 9150-9200, 9200-9250, 9250-9300, 9300-9350, 9350-9400, 9400-9450, 9450-9500, 9500-9550, 9550-9600, 9600-9650, 9650-9700, 9700-9750, 9750-9800, 9800-9850, 9850-9900, 9900-9950, 9950-10000, 10000-10050, 10050-10100, 10100-10150, 10150-10200, 10200-10250, 10250-10300, 10300-10350, 10350-10400, 10400-10450, 10450-10500, 10500-10550, 10550-10600, 10600-10650, 10650-10700, 10700-10750, 10750-10800, 10800-10850, 10850-10900, 10900-10950, 10950-11000, 11050-11100, 11100-11150, 11150-11200, 11200-11250, 11250-11300, 11300-11350, 11350-11400, 11400-11450, 11450-11500, 11500-11550, 11550-11600, 11600-11650, 11650-11700, 11700-11750, 11750-11800, 11800-11850, 11850-11900, 11900-11950, 11950-12000, 12000-12050, 12050-12100, 12100-12150, 12150-12200, 12200-12250, 12250-12300, 12300-12350, 12350-12400, 12400-12450, 12450-12500, 12500-12550, 12550-12600, 12600-12650, 12650-12700, 12700-12750, 12750-12800, 12800-12850, 12850-12900, 12900-12950, 12950-13000, 13050-13100, 13100-13150, 13150-13200, 13200-13250, 13250-13300, 13300-13350, 13350-13400, 13400-13450, and 13450-13500 on the target mRNA sequence. As yet another non-limiting example, the start site may be nucleotide 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1660, 1661, 1662, 1663, 1664, 1665, 1666, 1667, 1668, 1669, 1670, 1671, 1672, 1673, 1674, 1675, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4850, 4851, 4852, 4853, 4854, 4855, 4856, 4857, 4858, 4859, 4860, 4861, 4862, 4863, 4864, 4865, 4866, 4867, 4868, 4869, 4870, 4871, 4872, 4873, 4874, 4875, 4876, 4877, 4878, 4879, 4880, 4881, 4882, 4883, 4884, 4885, 4886, 4887, 4888, 4889, 4890, 4891, 4892, 4893, 4894, 4895, 4896, 4897, 4898, 4899, 4900, 5460, 5461, 5462, 5463, 5464, 5465, 5466, 5467, 5468, 5469, 5470, 5471, 5472, 5473, 5474, 5475, 5476, 5477, 5478, 5479, 5480, 6175, 6176, 6177, 6178, 6179, 6180, 6181, 6182, 6183, 6184, 6185, 6186, 6187, 6188, 6189, 6190, 6191, 6192, 6193, 6194, 6195, 6196, 6197, 6198, 6199, 6200, 6315, 6316, 6317, 6318, 6319, 6320, 6321, 6322, 6323, 6324, 6325, 6326, 6327, 6328, 6329, 6330, 6331, 6332, 6333, 6334, 6335, 6336, 6337, 6338, 6339, 6340, 6341, 6342, 6343, 6344, 6345, 6600, 6601, 6602, 6603, 6604, 6605, 6606, 6607, 6608, 6609, 6610, 6611, 6612, 6613, 6614, 6615, 6725, 6726, 6727, 6728, 6729, 6730, 6731, 6732, 6733, 6734, 6735, 6736, 6737, 6738, 6739, 6740, 6741, 6742, 6743, 6744, 6745, 6746, 6747, 6748, 6749, 6750, 6751, 6752, 6753, 6754, 6755, 6756, 6757, 6758, 6759, 6760, 6761, 6762, 6763, 6764, 6765, 6766, 6767, 6768, 6769, 6770, 6771, 6772, 6773, 6774, 6775, 7655, 7656, 7657, 7658, 7659, 7660, 7661, 7662, 7663, 7664, 7665, 7666, 7667, 7668, 7669, 7670, 7671, 7672, 8510, 8511, 8512, 8513, 8514, 8515, 8516, 8715, 8716, 8717, 8718, 8719, 8720, 8721, 8722, 8723, 8724, 8725, 8726, 8727, 8728, 8729, 8730, 8731, 8732, 8733, 8734, 8735, 8736, 8737, 8738, 8739, 8740, 8741, 8742, 8743, 8744, 8745, 9250, 9251, 9252, 9253, 9254, 9255, 9256, 9257, 9258, 9259, 9260, 9261, 9262, 9263, 9264, 9265, 9266, 9267, 9268, 9269, 9270, 9480, 9481, 9482, 9483, 9484, 9485, 9486, 9487, 9488, 9489, 9490, 9491, 9492, 9493, 9494, 9495, 9496, 9497, 9498, 9499, 9500, 9575, 9576, 9577, 9578, 9579, 9580, 9581, 9582, 9583, 9584, 9585, 9586, 9587, 9588, 9589, 9590, 10525, 10526, 10527, 10528, 10529, 10530, 10531, 10532, 10533, 10534, 10535, 10536, 10537, 10538, 10539, 10540, 11545, 11546, 11547, 11548, 11549, 11550, 11551, 11552, 11553, 11554, 11555, 11556, 11557, 11558, 11559, 11560, 11875, 11876, 11877, 11878, 11879, 11880, 11881, 11882, 11883, 11884, 11885, 11886, 11887, 11888, 11889, 11890, 11891, 11892, 11893, 11894, 11895, 11896, 11897, 11898, 11899, 11900, 11915, 11916, 11917, 11918, 11919, 11920, 11921, 11922, 11923, 11924, 11925, 11926, 11927, 11928, 11929, 11930, 11931, 11932, 11933, 11934, 11935, 11936, 11937, 11938, 11939, 11940, 13375, 13376, 13377, 13378, 13379, 13380, 13381, 13382, 13383, 13384, 13385, 13386, 13387, 13388, 13389 and 13390 on the target mRNA sequence.

In some embodiments, the antisense strand and target mRNA sequences have 100% complementarity. The antisense strand may be complementary to any part of the target mRNA sequence.

In other embodiments, the antisense strand and target mRNA sequences comprise at least one mismatch. As a non-limiting example, the antisense strand and the target mRNA sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementarity.

In one embodiment, an siRNA or dsRNA includes at least two sequences that are complementary to each other.

According to the present invention, the siRNA molecule has a length from about 10-50 or more nucleotides, i.e., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementarity to a target region. In one embodiment, each strand of the siRNA molecule has a length from about 19 to 25, 19 to 24 or 19 to 21 nucleotides. In one embodiment, at least one strand of the siRNA molecule is 19 nucleotides in length. In one embodiment, at least one strand of the siRNA molecule is 20 nucleotides in length. In one embodiment, at least one strand of the siRNA molecule is 21 nucleotides in length. In one embodiment, at least one strand of the siRNA molecule is 22 nucleotides in length. In one embodiment, at least one strand of the siRNA molecule is 23 nucleotides in length. In one embodiment, at least one strand of the siRNA molecule is 24 nucleotides in length. In one embodiment, at least one strand of the siRNA molecule is 25 nucleotides in length.\

In some embodiments, the siRNA molecules of the present invention can be synthetic RNA duplexes comprising about 19 nucleotides to about 25 nucleotides, and two overhanging nucleotides at the 3′-end. In some aspects, the siRNA molecules may be unmodified RNA molecules. In other aspects, the siRNA molecules may contain at least one modified nucleotide, such as base, sugar or backbone modifications.

In one embodiment, the siRNA molecules of the present invention may comprise an antisense sequence and a sense sequence, or a fragment or variant thereof. As a non-limiting example, the antisense sequence and the sense sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementarity.

In other embodiments, the siRNA molecules of the present invention can be encoded in plasmid vectors, AAV particles, viral genome or other nucleic acid expression vectors for delivery to a cell.

DNA expression plasmids can be used to stably express the siRNA duplexes or dsRNA of the present invention in cells and achieve long-term inhibition of the target gene expression. In one aspect, the sense and antisense strands of a siRNA duplex are typically linked by a short spacer sequence leading to the expression of a stem-loop structure termed short hairpin RNA (shRNA). The hairpin is recognized and cleaved by Dicer, thus generating mature siRNA molecules.

According to the present invention, AAV particles comprising the nucleic acids encoding the siRNA molecules targeting the mRNA are produced, the AAV serotypes may be any of the serotypes listed in Table 1. Non-limiting examples of the AAV serotypes include, AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ8, AAV-DJ, AAV-PHP.A, and/or AAV-PHP.B, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3, AAVG2B4, AAVG2B5 and variants thereof.

In some embodiments, the siRNA duplexes or encoded dsRNA of the present invention suppress (or degrade) the target mRNA. Accordingly, the siRNA duplexes or encoded dsRNA can be used to substantially inhibit the gene expression in a cell, for example a neuron. In some aspects, the inhibition of the gene expression refers to an inhibition by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In one embodiment, the siRNA molecules comprise a miRNA seed match for the target located in the guide strand. In another embodiment, the siRNA molecules comprise a miRNA seed match for the target located in the passenger strand. In yet another embodiment, the siRNA duplexes or encoded dsRNA targeting the gene of interest do not comprise a seed match for the target located in the guide or passenger strand.

In one embodiment, the siRNA duplexes or encoded dsRNA targeting the gene of interest may have almost no significant full-length off target effects for the guide strand. In another embodiment, the siRNA duplexes or encoded dsRNA targeting the gene of interest may have almost no significant full-length off target effects for the passenger strand. The siRNA duplexes or encoded dsRNA targeting the gene of interest may have less than 1%, 2%, 3%, 4%, 5%, 6%, 7%, 8%, 9%, 10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25% 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off target effects for the passenger strand. In yet another embodiment, the siRNA duplexes or encoded dsRNA targeting the gene of interest may have almost no significant full-length off target effects for the guide strand or the passenger strand. The siRNA duplexes or encoded dsRNA targeting the gene of interest may have less than 1%, 2%, 3%, 4%, 5%, 6%, 7%, 8%, 9%, 10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25% 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off target effects for the guide or passenger strand.

In one embodiment, the siRNA duplexes or encoded dsRNA targeting the gene of interest may have high activity in vitro. In another embodiment, the siRNA molecules may have low activity in vitro. In yet another embodiment, the siRNA duplexes or dsRNA targeting the gene of interest may have high guide strand activity and low passenger strand activity in vitro.

In one embodiment, the siRNA molecules have a high guide strand activity and low passenger strand activity in vitro. The target knock-down (KD) by the guide strand may be at least 40%, 50%, 60%, 65%, 70%, 75%, 80%, 85%, 90%, 95%, 99%, 99.5% or 100%. The target knock-down by the guide strand may be 40-50%, 45-50%, 50-55%, 50-60%, 60-65%, 60-70%, 60-75%, 60-80%, 60-85%, 60-90%, 60-95%, 60-99%, 60-99.5%, 60-100%, 65-70%, 65-75%, 65-80%, 65-85%, 65-90%, 65-95%, 65-99%, 65-99.5%, 65-100%, 70-75%, 70-80%, 70-85%, 70-90%, 70-95%, 70-99%, 70-99.5%, 70-100%, 75-80%, 75-85%, 75-90%, 75-95%, 75-99%, 75-99.5%, 75-100%, 80-85%, 80-90%, 80-95%, 80-99%, 80-99.5%, 80-100%, 85-90%, 85-95%, 85-99%, 85-99.5%, 85-100%, 90-95%, 90-99%, 90-99.5%, 90-100%, 95-99%, 95-99.5%, 95-100%, 99-99.5%, 99-100% or 99.5-100%. As a non-limiting example, the target knock-down (KD) by the guide strand is greater than 70%. As a non-limiting example, the target knock-down (KD) by the guide strand is greater than 60%.

In one embodiment, the siRNA duplex is designed so there is no miRNA seed match for the sense or antisense sequence to the non-gene of interest sequence.

In one embodiment, the IC₅₀ of the guide strand for the nearest off target is greater than 100 multiplied by the IC₅₀ of the guide strand for the on-target gene. As a non-limiting example, if the IC₅₀ of the guide strand for the nearest off target is greater than 100 multiplied by the IC₅₀ of the guide strand for the target then the siRNA molecule is said to have high guide strand selectivity for inhibiting the gene of interest in vitro.

In one embodiment, the 5′ processing of the guide strand has a correct start (n) at the 5′ end at least 75%, 80%, 85%, 90%, 95%, 99% or 100% of the time in vitro or in vivo. As a non-limiting example, the 5′ processing of the guide strand is precise and has a correct start (n) at the 5′ end at least 99% of the time in vitro. As a non-limiting example, the 5′ processing of the guide strand is precise and has a correct start (n) at the 5′ end at least 99% of the time in vivo. As a non-limiting example, the 5′ processing of the guide strand is precise and has a correct start (n) at the 5′ end at least 90% of the time in vitro. As a non-limiting example, the 5′ processing of the guide strand is precise and has a correct start (n) at the 5′ end at least 90% of the time in vivo. As a non-limiting example, the 5′ processing of the guide strand is precise and has a correct start (n) at the 5′ end at least 85% of the time in vitro. As a non-limiting example, the 5′ processing of the guide strand is precise and has a correct start (n) at the 5′ end at least 85% of the time in vivo.

In one embodiment, the guide to passenger (G:P) (also referred to as the antisense to sense) strand ratio expressed is 1:10, 1:9, 1:8, 1:7, 1:6, 1:5, 1:4, 1:3, 1:2, 1;1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The guide to passenger ratio refers to the ratio of the guide strands to the passenger strands after intracellular processing of the pri-microRNA. For example, a 80:20 of guide-to-passenger ratio would have 8 guide strands to every 2 passenger strands processed from the precursor. As a non-limiting example, the guide-to-passenger strand ratio is 8:2 in vitro. As a non-limiting example, the guide-to-passenger strand ratio is 8:2 in vivo. As a non-limiting example, the guide-to-passenger strand ratio is 9:1 in vitro. As a non-limiting example, the guide-to-passenger strand ratio is 9:1 in vivo.

In one embodiment, the guide to passenger (G:P) (also referred to as the antisense to sense) strand ratio expressed is greater than 1.

In one embodiment, the guide to passenger (G:P) (also referred to as the antisense to sense) strand ratio expressed is greater than 2.

In one embodiment, the guide to passenger (G:P) (also referred to as the antisense to sense) strand ratio expressed is greater than 5.

In one embodiment, the guide to passenger (G:P) (also referred to as the antisense to sense) strand ratio expressed is greater than 10.

In one embodiment, the guide to passenger (G:P) (also referred to as the antisense to sense) strand ratio expressed is greater than 20.

In one embodiment, the guide to passenger (G:P) (also referred to as the antisense to sense) strand ratio expressed is greater than 50.

In one embodiment, the guide to passenger (G:P) (also referred to as the antisense to sense) strand ratio expressed is at least 3:1.

In one embodiment, the guide to passenger (G:P) (also referred to as the antisense to sense) strand ratio expressed is at least 5:1.

In one embodiment, the guide to passenger (G:P) (also referred to as the antisense to sense) strand ratio expressed is at least 10:1.

In one embodiment, the guide to passenger (G:P) (also referred to as the antisense to sense) strand ratio expressed is at least 20:1.

In one embodiment, the guide to passenger (G:P) (also referred to as the antisense to sense) strand ratio expressed is at least 50:1.

In one embodiment, the passenger to guide (P:G) (also referred to as the sense to antisense) strand ratio expressed is 1:10, 1:9, 1:8, 1:7, 1:6, 1:5, 1:4, 1:3, 1:2, 1;1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The passenger to guide ratio refers to the ratio of the passenger strands to the guide strands after the intracellular processing of the pri-microRNA. For example, a 80:20 passenger-to-guide ratio would have 8 passenger strands to every 2 guide strands processed from the precursor. As a non-limiting example, the passenger-to-guide strand ratio is 80:20 in vitro. As a non-limiting example, the passenger-to-guide strand ratio is 80:20 in vivo. As a non-limiting example, the passenger-to-guide strand ratio is 8:2 in vitro. As a non-limiting example, the passenger-to-guide strand ratio is 8:2 in vivo. As a non-limiting example, the passenger-to-guide strand ratio is 9:1 in vitro. As a non-limiting example, the passenger-to-guide strand ratio is 9:1 in vivo.

In one embodiment, the passenger to guide (P:G) (also referred to as the sense to antisense) strand ratio expressed is greater than 1.

In one embodiment, the passenger to guide (P:G) (also referred to as the sense to antisense) strand ratio expressed is greater than 2.

In one embodiment, the passenger to guide (P:G) (also referred to as the sense to antisense) strand ratio expressed is greater than 5.

In one embodiment, the passenger to guide (P:G) (also referred to as the sense to antisense) strand ratio expressed is greater than 10.

In one embodiment, the passenger to guide (P:G) (also referred to as the sense to antisense) strand ratio expressed is greater than 20.

In one embodiment, the passenger to guide (P:G) (also referred to as the sense to antisense) strand ratio expressed is greater than 50.

In one embodiment, the passenger to guide (P:G) (also referred to as the sense to antisense) strand ratio expressed is at least 3:1.

In one embodiment, the passenger to guide (P:G) (also referred to as the sense to antisense) strand ratio expressed is at least 5:1.

In one embodiment, the passenger to guide (P:G) (also referred to as the sense to antisense) strand ratio expressed is at least 10:1.

In one embodiment, the passenger to guide (P:G) (also referred to as the sense to antisense) strand ratio expressed is at least 20:1.

In one embodiment, the passenger to guide (P:G) (also referred to as the sense to antisense) strand ratio expressed is at least 50:1.

In one embodiment, a passenger-guide strand duplex is considered effective when the pri- or pre-microRNAs demonstrate, but methods known in the art and described herein, greater than 2-fold guide to passenger strand ratio when processing is measured. As a non-limiting examples, the pri- or pre-microRNAs demonstrate great than 2-fold, 3-fold, 4-fold, 5-fold, 6-fold, 7-fold, 8-fold, 9-fold, 10-fold, 11-fold, 12-fold, 13-fold, 14-fold, 15-fold, or 2 to 5-fold, 2 to 10-fold, 2 to 15-fold, 3 to 5-fold, 3 to 10-fold, 3 to 15-fold, 4 to 5-fold, 4 to 10-fold, 4 to 15-fold, 5 to 10-fold, 5 to 15-fold, 6 to 10-fold, 6 to 15-fold, 7 to 10-fold, 7 to 15-fold, 8 to 10-fold, 8 to 15-fold, 9 to 10-fold, 9 to 15-fold, 10 to 15-fold, 11 to 15-fold, 12 to 15-fold, 13 to 15-fold, or 14 to 15-fold guide to passenger strand ratio when processing is measured.

In one embodiment, the vector genome encoding the dsRNA comprises a sequence which is at least 60%, 65%, 70%, 75%, 80%, 85%, 90%, 95%, 99% or more than 99% of the full length of the construct. As a non-limiting example, the vector genome comprises a sequence which is at least 80% of the full length sequence of the construct.

In one embodiment, the siRNA molecules may be used to silence wild type or mutant version of the gene of interest by targeting at least one exon on the gene of interest sequence. The exon may be exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, and/or exon 67.

Design and Sequences of siRNA Duplexes Targeting HTT Gene

The present invention provides small interfering RNA (siRNA) duplexes (and modulatory polynucleotides encoding them) that target HTT mRNA to interfere with HTT gene expression and/or HTT protein production.

The encoded siRNA duplex of the present invention contains an antisense strand and a sense strand hybridized together forming a duplex structure, wherein the antisense strand is complementary to the nucleic acid sequence of the targeted HTT gene, and wherein the sense strand is homologous to the nucleic acid sequence of the targeted HTT gene. In some aspects, the 5′end of the antisense strand has a 5′ phosphate group and the 3′end of the sense strand contains a 3′hydroxyl group. In other aspects, there are none, one or 2 nucleotide overhangs at the 3′end of each strand.

Some guidelines for designing siRNAs have been proposed in the art. These guidelines generally recommend generating a 19-nucleotide duplexed region, symmetric 2-3 nucleotide 3′ overhangs, 5′-phosphate and 3′-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such consideration, together with the specific sequence of a target gene, highly effective siRNA molecules essential for suppressing the Htt gene expression may be readily designed.

According to the present invention, siRNA molecules (e.g., siRNA duplexes or encoded dsRNA) that target the HTT gene are designed. Such siRNA molecules can specifically, suppress HTT gene expression and protein production. In some aspects, the siRNA molecules are designed and used to selectively “knock out” HTT gene variants in cells, i.e., mutated HTT transcripts that are identified in patients with HD disease. In some aspects, the siRNA molecules are designed and used to selectively “knock down” HTT gene variants in cells. In other aspects, the siRNA molecules are able to inhibit or suppress both the wild type and mutated HTT gene.

In one embodiment, an siRNA molecule of the present invention comprises a sense strand and a complementary antisense strand in which both strands are hybridized together to form a duplex structure. The antisense strand has sufficient complementarity to the HTT mRNA sequence to direct target-specific RNAi, i.e., the siRNA molecule has a sequence sufficient to trigger the destruction of the target mRNA by the RNAi machinery or process.

In one embodiment, an siRNA molecule of the present invention comprises a sense strand and a complementary antisense strand in which both strands are hybridized together to form a duplex structure and where the start site of the hybridization to the HTT mRNA is between nucleotide 100 and 7000 on the HTT mRNA sequence. As a non-limiting example, the start site may be between nucleotide 100-150, 150-200, 200-250, 250-300, 300-350, 350-400, 400-450, 450-500, 500-550, 550-600, 600-650, 650-700, 700-70, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, 3200-3250, 3250-3300, 3300-3350, 3350-3400, 3400-3450, 3450-3500, 3500-3550, 3550-3600, 3600-3650, 3650-3700, 3700-3750, 3750-3800, 3800-3850, 3850-3900, 3900-3950, 3950-4000, 4000-4050, 4050-4100, 4100-4150, 4150-4200, 4200-4250, 4250-4300, 4300-4350, 4350-4400, 4400-4450, 4450-4500, 4500-4550, 4550-4600, 4600-4650, 4650-4700, 4700-4750, 4750-4800, 4800-4850, 4850-4900, 4900-4950, 4950-5000, 5000-5050, 5050-5100, 5100-5150, 5150-5200, 5200-5250, 5250-5300, 5300-5350, 5350-5400, 5400-5450, 5450-5500, 5500-5550, 5550-5600, 5600-5650, 5650-5700, 5700-5750, 5750-5800, 5800-5850, 5850-5900, 5900-5950, 5950-6000, 6000-6050, 6050-6100, 6100-6150, 6150-6200, 6200-6250, 6250-6300, 6300-6350, 6350-6400, 6400-6450, 6450-6500, 6500-6550, 6550-6600, 6600-6650, 6650-6700, 6700-6750, 6750-6800, 6800-6850, 6850-6900, 6900-6950, 6950-7000, 7000-7050, 7050-7100, 7100-7150, 7150-7200, 7200-7250, 7250-7300, 7300-7350, 7350-7400, 7400-7450, 7450-7500, 7500-7550, 7550-7600, 7600-7650, 7650-7700, 7700-7750, 7750-7800, 7800-7850, 7850-7900, 7900-7950, 7950-8000, 8000-8050, 8050-8100, 8100-8150, 8150-8200, 8200-8250, 8250-8300, 8300-8350, 8350-8400, 8400-8450, 8450-8500, 8500-8550, 8550-8600, 8600-8650, 8650-8700, 8700-8750, 8750-8800, 8800-8850, 8850-8900, 8900-8950, 8950-9000, 9000-9050, 9050-9100, 9100-9150, 9150-9200, 9200-9250, 9250-9300, 9300-9350, 9350-9400, 9400-9450, 9450-9500, 9500-9550, 9550-9600, 9600-9650, 9650-9700, 9700-9750, 9750-9800, 9800-9850, 9850-9900, 9900-9950, 9950-10000, 10000-10050, 10050-10100, 10100-10150, 10150-10200, 10200-10250, 10250-10300, 10300-10350, 10350-10400, 10400-10450, 10450-10500, 10500-10550, 10550-10600, 10600-10650, 10650-10700, 10700-10750, 10750-10800, 10800-10850, 10850-10900, 10900-10950, 10950-11000, 11050-11100, 11100-11150, 11150-11200, 11200-11250, 11250-11300, 11300-11350, 11350-11400, 11400-11450, 11450-11500, 11500-11550, 11550-11600, 11600-11650, 11650-11700, 11700-11750, 11750-11800, 11800-11850, 11850-11900, 11900-11950, 11950-12000, 12000-12050, 12050-12100, 12100-12150, 12150-12200, 12200-12250, 12250-12300, 12300-12350, 12350-12400, 12400-12450, 12450-12500, 12500-12550, 12550-12600, 12600-12650, 12650-12700, 12700-12750, 12750-12800, 12800-12850, 12850-12900, 12900-12950, 12950-13000, 13050-13100, 13100-13150, 13150-13200, 13200-13250, 13250-13300, 13300-13350, 13350-13400, 13400-13450, and 13450-13500 on the HTT mRNA sequence. As yet another non-limiting example, the start site may be nucleotide 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 715, 716, 717, 718, 719, 720, 721, 722, 723, 724, 725, 875, 876, 877, 878, 879, 880, 881, 882, 883, 884, 885, 886, 887, 888, 889, 890, 891, 892, 893, 894, 895, 896, 897, 898, 899, 900, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1660, 1661, 1662, 1663, 1664, 1665, 1666, 1667, 1668, 1669, 1670, 1671, 1672, 1673, 1674, 1675, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4850, 4851, 4852, 4853, 4854, 4855, 4856, 4857, 4858, 4859, 4860, 4861, 4862, 4863, 4864, 4865, 4866, 4867, 4868, 4869, 4870, 4871, 4872, 4873, 4874, 4875, 4876, 4877, 4878, 4879, 4880, 4881, 4882, 4883, 4884, 4885, 4886, 4887, 4888, 4889, 4890, 4891, 4892, 4893, 4894, 4895, 4896, 4897, 4898, 4899, 4900, 5460, 5461, 5462, 5463, 5464, 5465, 5466, 5467, 5468, 5469, 5470, 5471, 5472, 5473, 5474, 5475, 5476, 5477, 5478, 5479, 5480, 6175, 6176, 6177, 6178, 6179, 6180, 6181, 6182, 6183, 6184, 6185, 6186, 6187, 6188, 6189, 6190, 6191, 6192, 6193, 6194, 6195, 6196, 6197, 6198, 6199, 6200, 6315, 6316, 6317, 6318, 6319, 6320, 6321, 6322, 6323, 6324, 6325, 6326, 6327, 6328, 6329, 6330, 6331, 6332, 6333, 6334, 6335, 6336, 6337, 6338, 6339, 6340, 6341, 6342, 6343, 6344, 6345, 6600, 6601, 6602, 6603, 6604, 6605, 6606, 6607, 6608, 6609, 6610, 6611, 6612, 6613, 6614, 6615, 6725, 6726, 6727, 6728, 6729, 6730, 6731, 6732, 6733, 6734, 6735, 6736, 6737, 6738, 6739, 6740, 6741, 6742, 6743, 6744, 6745, 6746, 6747, 6748, 6749, 6750, 6751, 6752, 6753, 6754, 6755, 6756, 6757, 6758, 6759, 6760, 6761, 6762, 6763, 6764, 6765, 6766, 6767, 6768, 6769, 6770, 6771, 6772, 6773, 6774, 6775, 7655, 7656, 7657, 7658, 7659, 7660, 7661, 7662, 7663, 7664, 7665, 7666, 7667, 7668, 7669, 7670, 7671, 7672, 8510, 8511, 8512, 8513, 8514, 8515, 8516, 8715, 8716, 8717, 8718, 8719, 8720, 8721, 8722, 8723, 8724, 8725, 8726, 8727, 8728, 8729, 8730, 8731, 8732, 8733, 8734, 8735, 8736, 8737, 8738, 8739, 8740, 8741, 8742, 8743, 8744, 8745, 9250, 9251, 9252, 9253, 9254, 9255, 9256, 9257, 9258, 9259, 9260, 9261, 9262, 9263, 9264, 9265, 9266, 9267, 9268, 9269, 9270, 9480, 9481, 9482, 9483, 9484, 9485, 9486, 9487, 9488, 9489, 9490, 9491, 9492, 9493, 9494, 9495, 9496, 9497, 9498, 9499, 9500, 9575, 9576, 9577, 9578, 9579, 9580, 9581, 9582, 9583, 9584, 9585, 9586, 9587, 9588, 9589, 9590, 10525, 10526, 10527, 10528, 10529, 10530, 10531, 10532, 10533, 10534, 10535, 10536, 10537, 10538, 10539, 10540, 11545, 11546, 11547, 11548, 11549, 11550, 11551, 11552, 11553, 11554, 11555, 11556, 11557, 11558, 11559, 11560, 11875, 11876, 11877, 11878, 11879, 11880, 11881, 11882, 11883, 11884, 11885, 11886, 11887, 11888, 11889, 11890, 11891, 11892, 11893, 11894, 11895, 11896, 11897, 11898, 11899, 11900, 11915, 11916, 11917, 11918, 11919, 11920, 11921, 11922, 11923, 11924, 11925, 11926, 11927, 11928, 11929, 11930, 11931, 11932, 11933, 11934, 11935, 11936, 11937, 11938, 11939, 11940, 13375, 13376, 13377, 13378, 13379, 13380, 13381, 13382, 13383, 13384, 13385, 13386, 13387, 13388, 13389 and 13390 on the HTT mRNA sequence.

In some embodiments, the antisense strand and target Htt mRNA sequences have 100% complementarity. The antisense strand may be complementary to any part of the target Htt mRNA sequence.

In other embodiments, the antisense strand and target Htt mRNA sequences comprise at least one mismatch. As a non-limiting example, the antisense strand and the target Htt mRNA sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementarity.

In one embodiment, an siRNA or dsRNA targeting Htt includes at least two sequences that are complementary to each other.

According to the present invention, the siRNA molecule targeting Htt has a length from about 10-50 or more nucleotides, i.e., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementarity to a target region. In one embodiment, each strand of the siRNA molecule has a length from about 19 to 25, 19 to 24 or 19 to 21 nucleotides. In one embodiment, at least one strand of the siRNA molecule is 19 nucleotides in length. In one embodiment, at least one strand of the siRNA molecule is 20 nucleotides in length. In one embodiment, at least one strand of the siRNA molecule is 21 nucleotides in length. In one embodiment, at least one strand of the siRNA molecule is 22 nucleotides in length. In one embodiment, at least one strand of the siRNA molecule is 23 nucleotides in length. In one embodiment, at least one strand of the siRNA molecule is 24 nucleotides in length. In one embodiment, at least one strand of the siRNA molecule is 25 nucleotides in length.

In some embodiments, the siRNA molecules of the present invention targeting Htt can be synthetic RNA duplexes comprising about 19 nucleotides to about 25 nucleotides, and two overhanging nucleotides at the 3′-end. In some aspects, the siRNA molecules may be unmodified RNA molecules. In other aspects, the siRNA molecules may contain at least one modified nucleotide, such as base, sugar or backbone modifications.

In one embodiment, the siRNA molecules of the present invention targeting Htt may comprise a nucleotide sequence such as, but not limited to, the antisense (guide) sequences in Table 2 or a fragment or variant thereof. As a non-limiting example, the antisense sequence used in the siRNA molecule of the present invention is at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% of a nucleotide sequence in Table 2. As another non-limiting example, the antisense sequence used in the siRNA molecule of the present invention comprises at least 3, 4, 5, 6, 7, 8, 9, 10, 11, 12, 13, 14, 15, 16, 17, 18, 19, 20, 21 or more than 21 consecutive nucleotides of a nucleotide sequence in Table 2. As yet another non-limiting example, the antisense sequence used in the siRNA molecule of the present invention comprises nucleotides 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 5 to 22, 5 to 21, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 6 to 22, 6 to 21, 6 to 20, 6 to 19, 6 to 18, 6 to 17, 6 to 16, 6 to 15, 6 to 14, 6 to 13, 6 to 12, 6 to 11, 6 to 10, 7 to 22, 7 to 21, 7 to 20, 7 to 19, 7 to 18, 7 to 17, 7 to 16, 7 to 15, 7 to 14, 7 to 13, 7 to 12, 8 to 22, 8 to 21, 8 to 20, 8 to 19, 8 to 18, 8 to 17, 8 to 16, 8 to 15, 8 to 14, 8 to 13, 8 to 12, 9 to 22, 9 to 21, 9 to 20, 9 to 19, 9 to 18, 9 to 17, 9 to 16, 9 to 15, 9 to 14, 10 to 22, 10 to 21, 10 to 20, 10 to 19, 10 to 18, 10 to 17, 10 to 16, 10 to 15, 10 to 14, 11 to 22, 11 to 21, 11 to 20, 11 to 19, 11 to 18, 11 to 17, 11 to 16, 11 to 15, 11 to 14, 12 to 22, 12 to 21, 12 to 20, 12 to 19, 12 to 18, 12 to 17, 12 to 16, 13 to 22, 13 to 21, 13 to 20, 13 to 19, 13 to 18, 13 to 17, 13 to 16, 14 to 22, 14 to 21, 14 to 20, 14 to 19, 14 to 18, 14 to 17, 15 to 22, 15 to 21, 15 to 20, 15 to 19, 15 to 18, 16 to 22, 16 to 21, 16 to 20, 17 to 22, 17 to 21, or 18 to 22 of the sequences in Table 2.

TABLE 2
Antisense Sequences

| Antisense | | SEQ |
|---|---|---|
| ID | Sequence | ID NO |
| A-2000 | UUAACGUCAGUUCAUAAACUU | 916 |
| A-2000dt | UUAACGUCAGUUCAUAAACdTdT | 917 |
| A-2001 | UGUCGGUACCGUCUAACACUU | 918 |
| A-2001dt | UGUCGGUACCGUCUAACACdTdT | 919 |
| A-2002 | UAAGCAUGGAGCUAGCAGGUU | 920 |
| A-2002dt | UAAGCAUGGAGCUAGCAGGdTdT | 921 |
| A-2003 | UACAACGAGACUGAAUUGCUU | 922 |
| A-2003dt | UACAACGAGACUGAAUUGCdTdT | 923 |
| A-2004 | UUCAGUUCAUAAACCUGGAUU | 924 |
| A-2004dt | UUCAGUUCAUAAACCUGGAdTdT | 925 |
| A-2005 | UAACGUCAGUUCAUAAACCUU | 926 |
| A-2005dt | UAACGUCAGUUCAUAAACCdTdT | 927 |
| A-2006 | UCCGGUCACAACAUUGUGGUU | 928 |
| A-2006dt | UCCGGUCACAACAUUGUGGdTdT | 929 |
| A-2007 | UUGCACGGUUCUUUGUGACUU | 930 |
| A-2007dt | UUGCACGGUUCUUUGUGACdTdT | 931 |
| A-2008 | UUUUAUAACAAGAGGUUCAUU | 932 |
| A-2008dt | UUUUAUAACAAGAGGUUCAdTdT | 933 |
| A-2009 | UCCAAAUACUGGUUGUCGGUU | 934 |
| A-2009dt | UCCAAAUACUGGUUGUCGGdTdT | 935 |
| A-2010 | UAUUUUAGGAAUUCCAAUGUU | 936 |
| A-2010dt | UAUUUUAGGAAUUCCAAUGdTdT | 937 |
| A-2011 | UUUAGGAAUUCCAAUGAUCUU | 938 |
| A-2011dt | UUUAGGAAUUCCAAUGAUCdTdT | 939 |
| A-2012dt | UUAAUCUCUUUACUGAUAUdTdT | 940 |
| A-2013dt | GAUUUUAGGAAUUCCAAUGdTdT | 941 |
| A-2014 | UAAGCAUGGAGCUAGCAGGCUU | 942 |
| A-2015 | UAAGCAUGGAGCUAGCAGGGU | 943 |
| A-2016 | AAGGACUUGAGGGACUCGAAGU | 944 |
| A-2017 | AAGGACUUGAGGGACUCGAAG | 945 |
| A-2018 | AAGGACUUGAGGGACUCGA | 946 |
| A-2019 | AGGACUUGAGGGACUCGAAGU | 947 |
| A-2020 | GAGGACUUGAGGGACUCGAAGU | 948 |
| A-2021 | AAGGACUUGAGGGACUCGAAGU | 949 |
| A-2022 | AAGGACUUGAGGGACUCGAAGUU | 950 |
| A-2023 | AAGGACUUGAGGGACUCGAAG | 951 |
| A-2024 | AAGGACUUGAGGGACUCGA | 952 |
| A-2025 | AAGGACUUGAGGGACUCGAAGG | 953 |
| A-2026 | AAGGACUUGAGGGACUCGAAU | 954 |
| A-2027 | AAGGACUUGAGGGACUCGAAGA | 955 |
| A-2028 | AAGGACUUGAGGGACUCGAAGG | 956 |
| A-2029 | AAGGACUUGAGGGACUCGAAGGU | 957 |
| A-2030 | AAGGACUUGAGGGACUCGAAGGA | 958 |
| A-2031 | AAGGACUUGAGGGACUCGAAG | 959 |
| A-2032 | AAGGACUUGAGGGACUCGAAGU | 960 |
| A-2033 | AAGGACUUGAGGGACUCGA | 961 |
| A-2034 | AAGGACUUGAGGGACUCGAAGGA | 962 |
| A-2035 | AAGGACUUGAGGGACUCGAAGG | 963 |
| A-2036 | AAGGACUUGAGGGACUCGAAGGAU | 964 |
| A-2037 | AAGGACUUGAGGGACUCGAAGGAUU | 965 |
| A-2038 | AAGGACUUGAGGGACUCGAAG | 966 |
| A-2039 | AAGGACUUGAGGGACUCGAAGGAA | 967 |
| A-2040 | GAUGAAGUGCACACAUUGGAUGA | 968 |
| A-2041 | GAUGAACUGCACACAUUGGAUG | 969 |
| A-2042 | GAUGAAUUGCACACAGUAGAUGA | 970 |
| A-2043 | AAGGACUUGAGGGACUCGAAGGUU | 971 |
| A-2044 | AAGGACUUGAGGGACUCGAAGGUUU | 972 |
| A-2045 | AAGGACUUGAGGGACUCGAAGGU | 973 |
| A-2046 | AAGGACUUGAGGGACUCGAAGGUUUU | 974 |
| A-2047 | AAGGACUUGAGGGACUCGAAGGUUUUU | 975 |
| A-2048 | AAGGACUUGAGGGACUCGAAGG | 976 |
| A-2049 | UAAGGACUUGAGGGACUCGAAG | 977 |
| A-2050 | AAGGACUUGAGGGACUCGAAG | 978 |
| A-2051 | AAGGACUUGAGGGACUCGAAGU | 979 |
| A-2052 | AAGGACUUGAGGGACUCGAAGACGA | 980 |
| | GUCCC | |
| A-2053 | AAGGACUUGAGGGACUCGAAGACGA | 981 |
| | GUCCCA | |
| A-2054 | AAGGACUUGAGGGACUCGAAGACG | 982 |
| | AGUCCCU | |
| A-2055 | GAUGAAGUGCACACAUUGGAUAC | 983 |
| A-2056 | GAUGAAGUGCACACAUUGGAUACA | 984 |
| A-2057 | GAUGAAGUGCACACAUUGGAUACA | 985 |
| | AUGUGU | |
| A-2058 | GAUGAAGUGCACACAUUGGAU | 986 |
| A-2059 | GAUGAAGUGCACACAUUGGAUA | 987 |
| A-2060 | GAUGAAUUGCACACAGUAGAUAU | 988 |
| A-2061 | GAUGAAUUGCACACAGUAGAUAUAC | 989 |
| A-2062 | GAUGAAUUGCACACAGUAGAUAUA | 990 |
| | CUGUGU | |
| A-2063 | GAUGAAUUGCACACAGUAGAUAUA | 991 |
| A-2064 | AUGAAUUGCACACAGUAGAUAUAC | 992 |
| A-2065 | GAUGAAUUGCACACAGUAGAUA | 993 |
| A-2066 | GAUGAAUUGCACACAGUAGAUAU | 994 |
| | ACUGUGU | |
| A-2067 | UACAACGAGACUGAAUUGCU | 995 |
| A-2068 | ACAACGAGACUGAAUUGCUU | 996 |
| A-2069 | UCCGGUCACAACAUUGUGGUUC | 997 |
| A-2070 | UCCGGUCACAACAUUGUGGU | 998 |
| A-2071 | UCCGGUCACAACAUUGUG | 999 |
| A-2072 | CCGGUCACAACAUUGUGGUU | 1000 |
| A-2073 | UUUUAUAACAAGAGGUUCAU | 1001 |
| A-2074 | UUUAUAACAAGAGGUUCAUU | 1002 |
| A-2075 | UAAGCAUGGAGCUAGCAGGU | 1003 |
| A-2076 | AAGCAUGGAGCUAGCAGGUU | 1004 |
| A-2077 | CCAAAUACUGGUUGUCGGUU | 1005 |
| A-2078 | UACAACGAGACUGAAUUGCUUU | 1006 |
| A-2079 | UAACGUCAGUUCAUAAACCUUU | 1007 |
| A-2080 | GUCCGGUCACAACAUUGUGGUU | 1008 |
| A-2081 | UCCGGUCACAACAUUGUGGUUUG | 1009 |
| A-2082 | UCCGGUCACAACAUUGUGGUUU | 1010 |
| A-2083 | UCCGGUCACAACAUUGUGG | 1011 |
| A-2084 | UAAGCAUGGAGCUAGCAGGUUU | 1012 |
| A-2085 | AAGCAUGGAGCUAGCAGGUUU | 1013 |
| A-2086 | UCCAAAUACUGGUUGUCGGUUU | 1014 |
| A-2087 | CCAAAUACUGGUUGUCGGUUU | 1015 |

In one embodiment, the siRNA molecules of the present invention targeting Htt may comprise a nucleotide sequence such as, but not limited to, the sense (passenger) sequences in Table 3 or a fragment or variant thereof. As a non-limiting example, the sense sequence used in the siRNA molecule of the present invention is at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% of a nucleotide sequence in Table 3. As another non-limiting example, the sense sequence used in the siRNA molecule of the present invention comprises at least 3, 4, 5, 6, 7, 8, 9, 10, 11, 12, 13, 14, 15, 16, 17, 18, 19, 20, 21 or more than 21 consecutive nucleotides of a nucleotide sequence in Table 3. As yet another non-limiting example, the sense sequence used in the siRNA molecule of the present invention comprises nucleotides 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 5 to 22, 5 to 21, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 6 to 22, 6 to 21, 6 to 20, 6 to 19, 6 to 18, 6 to 17, 6 to 16, 6 to 15, 6 to 14, 6 to 13, 6 to 12, 6 to 11, 6 to 10, 7 to 22, 7 to 21, 7 to 20, 7 to 19, 7 to 18, 7 to 17, 7 to 16, 7 to 15, 7 to 14, 7 to 13, 7 to 12, 8 to 22, 8 to 21, 8 to 20, 8 to 19, 8 to 18, 8 to 17, 8 to 16, 8 to 15, 8 to 14, 8 to 13, 8 to 12, 9 to 22, 9 to 21, 9 to 20, 9 to 19, 9 to 18, 9 to 17, 9 to 16, 9 to 15, 9 to 14, 10 to 22, 10 to 21, 10 to 20, 10 to 19, 10 to 18, 10 to 17, 10 to 16, 10 to 15, 10 to 14, 11 to 22, 11 to 21, 11 to 20, 11 to 19, 11 to 18, 11 to 17, 11 to 16, 11 to 15, 11 to 14, 12 to 22, 12 to 21, 12 to 20, 12 to 19, 12 to 18, 12 to 17, 12 to 16, 13 to 22, 13 to 21, 13 to 20, 13 to 19, 13 to 18, 13 to 17, 13 to 16, 14 to 22, 14 to 21, 14 to 20, 14 to 19, 14 to 18, 14 to 17, 15 to 22, 15 to 21, 15 to 20, 15 to 19, 15 to 18, 16 to 22, 16 to 21, 16 to 20, 17 to 22, 17 to 21, or 18 to 22 of the sequences in Table 3.

TABLE 3
Sense Sequences

| | | SEQ |
|---|---|---|
| | | ID |
| Sense ID | Sequence | NO |
| S-1000 | GUUUAUGAACUGAUCUUACCC | 1016 |
| S-1001 | GUGUUAGACGGUACUGAUCCC | 1017 |
| S-1002 | CCUGCUAGCUCCAUGCUUCCC | 1018 |
| S-1003 | GUUUAUGAACUGAUCUUAGCC | 1019 |
| S-1004 | GUGUUAGACGGUACUGAUGCC | 1020 |
| S-1005 | CCUGCUAGCUCCAUGCUUGCC | 1021 |
| S-1006 | GUUUAUGAAGUGAUCUUAACC | 1022 |
| S-1007 | GUGUUAGACCGUACUGAUACC | 1023 |
| S-1008 | CCUGCUAGCACCAUGCUUACC | 1024 |
| S-1009 | GUUUAUGAAGUGAUCUUAACC | 1025 |
| S-1010 | GUGUUAGACGGUACUGAUACC | 1026 |
| S-1011 | CCUGCUAGCUCCAUGCUUACC | 1027 |
| S-lOlldt | CCUGCUAGCUCCAUGCUUAdTdT | 1028 |
| S-1012 | GUUUAUGAACUGAUCUUGCCC | 1029 |
| S-1013 | GUUUAUGAACUGAUCUUGGCC | 1030 |
| S-1014 | GUUUAUGAACUGAUCUUGACC | 1031 |
| S-1015 | GCAAUUCAGUCUCGUUGUCCC | 1032 |
| S-1016 | UCCAGGUUUAUGAACUGACCC | 1033 |
| S-1017 | GGUUUAUGAACUGACGUUCCC | 1034 |
| S-1018 | CCACAAUGUUGUGACUGGCCC | 1035 |
| S-1019 | GUCACAAAGAACCGUGUACCC | 1036 |
| S-1020 | UGAACCUCUUGUUAUAAACCC | 1037 |
| S-1021 | CCGACAACCAGUAUUUGGCCC | 1038 |
| S-1022 | GCAAUUCAGUCUCGUUGUGCC | 1039 |
| S-1023 | UCCAGGUUUAUGAACUGAGCC | 1040 |
| S-1024 | GGUUUAUGAACUGACGUUGCC | 1041 |
| S-1025 | CCACAAUGUUGUGACUGGGCC | 1042 |
| S-1026 | GUCACAAAGAACCGUGUAGCC | 1043 |
| S-1027 | UGAACCUCUUGUUAUAAAGCC | 1044 |
| S-1028 | CCGACAACCAGUAUUUGGGCC | 1045 |
| S-1029 | GCAAUUCAGUCUCGUUGUACC | 1046 |
| S-1029dt | GCAAUUCAGUCUCGUUGUAdTdT | 1047 |
| S-1030 | UCCAGGUUUAUGAACUGAACC | 1048 |
| S-lO3Odt | UCCAGGUUUAUGAACUGAAdTdT | 1049 |
| S-1031 | GGUUUAUGAACUGACGUUACC | 1050 |
| S-1032 | CCACAAUGUUGUGACUGGACC | 1051 |
| S-1033 | GUCACAAAGAACCGUGUAACC | 1052 |
| S-1034 | UGAACCUCUUGUUAUAAAACC | 1053 |
| S-1034dt | UGAACCUCUUGUUAUAAAAdTdT | 1054 |
| S-1035 | CCGACAACCAGUAUUUGGACC | 1055 |
| S-1035dt | CCGACAACCAGUAUUUGGAdTdT | 1056 |
| S-1036 | GCAAUUCAGACUCGUUGUACC | 1057 |
| S-1037 | UCCAGGUUUUUGAACUGAACC | 1058 |
| S-1038 | GGUUUAUGAUCUGACGUUACC | 1059 |
| S-1039 | CCACAAUGUAGUGACUGGACC | 1060 |
| S-1040 | GUCACAAAGUACCGUGUAACC | 1061 |
| S-1041 | UGAACCUCUAGUUAUAAAACC | 1062 |
| S-1042 | CCGACAACCUGUAUUUGGACC | 1063 |
| S-1043 | CAUUGGAAUUCCUAAAAUUCC | 1064 |
| S-1044 | GAUCAUUGGAAUUCCUAAUCC | 1065 |
| S-1045 | CAUUGGAAUUCCUAAAAUGCC | 1066 |
| S-1046 | GAUCAUUGGAAUUCCUAAGCC | 1067 |
| S-1047 | CAUUGGAAUUCCUAAAAUACC | 1068 |
| S-1047dt | CAUUGGAAUUCCUAAAAUAdTdT | 1069 |
| S-1048 | GAUCAUUGGAAUUCCUAAACC | 1070 |
| S-1048dt | GAUCAUUGGAAUUCCUAAAdTdT | 1071 |
| S-1049 | CAUUGGAAUACCUAAAAUACC | 1072 |
| S-1050 | GAUCAUUGGUAUUCCUAAACC | 1073 |
| S-1051dt | GUUUAUGAACUGACGUUAAdTdT | 1074 |
| S-1052dt | GUGUUAGACGGUACCGACAdTdT | 1075 |
| S-1053dt | AUAUCAGUAAAGAGAUUAAdTdT | 1076 |
| S-1054dt | GGUUUAUGAACUGACGUUAdTdT | 1077 |
| S-1055dt | CCACAAUGUUGUGACCGGAdTdT | 1078 |
| S-1056dt | GUCACAAAGAACCGUGCAAdTdT | 1079 |
| S-1057dt | CAUUGGAAUUCCUAAAAUCdTdT | 1080 |
| S-1058 | CCUGCUAGCUCCAUGCUUGCU | 1081 |
| S-1059 | CCUGCUAGCUCCAUGCUUGAU | 1082 |
| S-1060 | CCUGCUAGCUCCAUGCUUAUU | 1083 |
| S-1061 | CCUGCUAGCUCCAUGCUUGUU | 1084 |
| S-1062 | UUCGAGUCCCUCAAGUAGCU | 1085 |
| S-1063 | UUCGAGUCCCUCAAGUAGCUUU | 1086 |
| S-1064 | UCGAGUCCCUCAAGUCCAUUCU | 1087 |
| S-1065 | UUCCAGUCCAUCAAGUCAAUU | 1088 |
| S-1066 | UUCCGAGUCUAAAAGUCCUUGG | 1089 |
| S-1067 | UUCCGAGUCUAAAAGUCCUUGGC | 1090 |
| S-1068 | CUUCCGAGUCUAAAAGUCCUUGG | 1091 |
| S-1069 | UUCCGAGUCUAAAAGUCCUUGGU | 1092 |
| S-1070 | UUCCGAGUCUAAAAGUCCUU | 1093 |
| | GGCU | |
| S-1071 | UCCAAUGUGAAACUUCAUCGGCU | 1094 |
| S-1072 | UCCAAUGUGAAACUUCAUCGGC | 1095 |
| S-1073 | AUCCAAUGUGAAACUUCAUCGU | 1096 |
| S-1074 | AUCCAAUGUGAAACUUCAUCGGU | 1097 |
| S-1075 | UCCAAUGUGAAACUUCAUCGGU | 1098 |
| S-1076 | UCCAAUGUGAAACUUCAUCGG | 1099 |
| | CUU | |
| S-1077 | AUCUACUGUGAAAAUUCAUCGG | 1100 |
| S-1078 | UCUACUGUGAAAAUUCAUCGG | 1101 |
| S-1079 | UCUACUGUGAAAAUUCAUCGGC | 1102 |
| S-1080 | AUCUACUGUGAAAAUUCAUCGGU | 1103 |
| S-1081 | UCUACUGUGAAAAUUCAUCGGU | 1104 |
| S-1082 | UCUACUGUGAAAAUUCAUCGGCU | 1105 |
| S-1083 | CCUUCGGUCCUCAAGUCCUUCA | 1106 |
| S-1084 | UUCGAGUCCAUCAAAUCCUAUAGU | 1107 |
| S-1085 | UACAAUGUGUGCACUUCAUAU | 1108 |
| S-1086 | UAUACUGUGUGCAAUUCAUUUCU | 1109 |
| S-1087 | GCAAUUCAGUCUCGUUGUCC | 1110 |
| S-1088 | GCAAUUCAGUCUCGUUGUC | 1111 |
| S-1089 | CAAUUCAGUCUCGUUGUCCC | 1112 |
| S-1090 | CAAUUCAGUCUCGUUGUCC | 1113 |
| S-1091 | GCAAUUCAGUCUCGUUGUGC | 1114 |
| S-1092 | CAAUUCAGUCUCGUUGUGCC | 1115 |
| S-1093 | CCACAAUGUUGUGACUGGGCCU | 1116 |
| S-1094 | CCACAAUGUUGUGACUGGGC | 1117 |
| S-1095 | CACAAUGUUGUGACUGGGCC | 1118 |
| S-1096 | UGAACCUCUUGUUAUAAAGCCU | 1119 |
| S-1097 | UGAACCUCUUGUUAUAAAGC | 1120 |
| S-1098 | GAACCUCUUGUUAUAAAGCC | 1121 |
| S-1099 | CCUGCUAGCUCCAUGCUUGCCU | 1122 |
| S-1100 | CCUGCUAGCUCCAUGCUUGC | 1123 |
| S-1101 | CCUGCUAGCUCCAUGCUUG | 1124 |
| S-1102 | CUGCUAGCUCCAUGCUUGCC | 1125 |
| S-1103 | CCGACAACCAGUAUUUGGGCCU | 1126 |
| S-1104 | CCGACAACCAGUAUUUGGGC | 1127 |
| S-1105 | CCGACAACCAGUAUUUGGG | 1128 |
| S-1106 | CGACAACCAGUAUUUGGGCC | 1129 |
| S-1107 | CGACAACCAGUAUUUGGGC | 1130 |
| S-1108 | GCAAUUCAGUCUCGUUGUACCU | 1131 |
| S-1109 | GCAAUUCAGUCUCGUUGUAC | 1132 |
| S-1110 | GCAAUUCAGUCUCGUUGUA | 1133 |
| S-1111 | CAAUUCAGUCUCGUUGUACC | 1134 |
| S-1112 | GCAAUUCAGACUCGUUGUACCU | 1135 |
| S-1113 | GCAAUUCAGACUCGUUGUAC | 1136 |
| S-1114 | GCAAUUCAGACUCGUUGUA | 1137 |
| S-1115 | CAAUUCAGACUCGUUGUACC | 1138 |
| S-1116 | AGCAAUUCAGUCUCGUUGUACC | 1139 |
| S-1117 | AGCAAUUCAGUCUCGUUGUAC | 1140 |
| S-1118 | AGGUUUAUGAACUGACGUUAC | 1141 |
| S-1119 | AGGUUUAUGAACUGACGUUACC | 1142 |
| S-1120 | ACCACAAUGUUGUGACUGGAC | 1143 |
| S-1121 | ACCACAAUGUUGUGACUGGACC | 1144 |
| S-1122 | CCACAAUGUUGUGACUGGACCGU | 1145 |
| S-1123 | CCACAAUGUUGUGACUGGACCG | 1146 |
| S-1124 | CCACAAUGUUGUGACUGGAC | 1147 |
| S-1125 | CACAAUGUUGUGACUGGACC | 1148 |
| S-1126 | ACCUGCUAGCUCCAUGCUUCCC | 1149 |
| S-1127 | ACCUGCUAGCUCCAUGCUUCC | 1150 |
| S-1128 | ACCUGCUAGCUCCAUGCUUC | 1151 |
| S-1129 | CCUGCUAGCUCCAUGCUUCC | 1152 |
| S-1130 | CCUGCUAGCUCCAUGCUUC | 1153 |
| S-1131 | CUGCUAGCUCCAUGCUUCCC | 1154 |
| S-1132 | CUGCUAGCUCCAUGCUUCC | 1155 |
| S-1133 | ACCGACAACCAGUAUUUGGACC | 1156 |
| S-1134 | ACCGACAACCAGUAUUUGGAC | 1157 |
| S-1135 | CCGACAACCAGUAUUUGGACCGU | 1158 |
| S-1136 | CCGACAACCAGUAUUUGGACCGU | 1159 |
| S-1137 | CCGACAACCAGUAUUUGGAC | 1160 |
| S-1138 | CGACAACCAGUAUUUGGACC | 1161 |
| S-1139 | CCUGCUAGCACCGUGCUUACC | 1162 |

In one embodiment, the siRNA molecules of the present invention targeting Htt may comprise an antisense sequence from Table 2 and a sense sequence from Table 3, or a fragment or variant thereof. As a non-limiting example, the antisense sequence and the sense sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementarity.

In one embodiment, the siRNA molecules of the present invention targeting Htt may comprise the sense and antisense siRNA duplex as described in Tables 4-6. As a non-limiting example, these siRNA duplexes may be tested for in vitro inhibitory activity on endogenous HTT gene expression. The start site for the sense and antisense sequence is compared to HTT gene sequence known as NM_002111.7 (SEQ ID NO: 1163) from NCBI.

TABLE 4
Sense and antisense strand sequences of HTT dsRNA

| | | | Sense | | | | Antisense | |
|---|---|---|---|---|---|---|---|---|
| siRNA | | | Strand | SS | | | Strand | AS |
| Duplex | SS | Start | Sequence | SEQ | | Start | Sequence | SEQ |
| ID | ID | SS | (5′-3′) | ID | AS ID | AS | (5′-3′) | ID |
| D-3566 | S- | 6751 | CCUGCUAGCUCCA | 1081 | A-2002 | 6751 | UAAGCAUGGAGCU | 920 |
| | 1058 | | UGCUUGCU | | | | AGCAGGUU | |
| D-3567 | S- | 6751 | CCUGCUAGCUCCA | 1081 | A-2014 | 6748 | UAAGCAUGGAGCU | 942 |
| | 1058 | | UGCUUGCU | | | | AGCAGGCUU | |
| D-3568 | S- | 6751 | CCUGCUAGCUCCA | 1082 | A-2002 | 6751 | UAAGCAUGGAGCU | 920 |
| | 1059 | | UGCUUGAU | | | | AGCAGGUU | |
| D-3569 | S- | 6751 | CCUGCUAGCUCCA | 1083 | A-2015 | 6751 | UAAGCAUGGAGCU | 943 |
| | 1060 | | UGCUUAUU | | | | AGCAGGGU | |
| D-3570 | S- | 6751 | CCUGCUAGCUCCA | 1084 | A-2002 | 6751 | UAAGCAUGGAGCU | 920 |
| | 1061 | | UGCUUGUU | | | | AGCAGGUU | |
| D-3500 | S- | 1386 | UCCAGGUUUAUGA | 1033 | A-2004 | 1386 | UUCAGUUCAUAAA | 924 |
| | 1016 | | ACUGACCC | | | | CCUGGAUU | |
| D-3501 | S- | 1386 | UCCAGGUUUAUGA | 1040 | A-2004 | 1386 | UUCAGUUCAUAAA | 924 |
| | 1023 | | ACUGAGCC | | | | CCUGGAUU | |
| D-3502 | S- | 1386 | UCCAGGUUUAUGA | 1048 | A-2004 | 1386 | UUCAGUUCAUAAA | 924 |
| | 1030 | | ACUGAACC | | | | CCUGGAUU | |
| D-3503 | S- | 1386 | UCCAGGUUUUUG | 1058 | A-2004 | 1386 | UUCAGUUCAUAAA | 924 |
| | 1037 | | AACUGAACC | | | | CCUGGAUU | |
| D-3504 | S- | 1386 | UCCAGGUUUAUGA | 1048 | A-2001 | 2066 | UGUCGGUACCGUC | 918 |
| | 1030 | | ACUGAACC | | | | UAACACUU | |
| D-3505 | S- | 1390 | GGUUUAUGAACU | 1034 | A-2005 | 1389 | UAACGUCAGUUCA | 926 |
| | 1017 | | GACGUUCCC | | | | UAAACCUU | |
| D-3506 | S- | 1390 | GGUUUAUGAACU | 1041 | A-2005 | 1389 | UAACGUCAGUUCA | 926 |
| | 1024 | | GACGUUGCC | | | | UAAACCUU | |
| D-3507 | S- | 1390 | GGUUUAUGAACU | 1050 | A-2005 | 1389 | UAACGUCAGUUCA | 926 |
| | 1031 | | GACGUUACC | | | | UAAACCUU | |
| D-3508 | S- | 1390 | GGUUUAUGAUCU | 1059 | A-2005 | 1389 | UAACGUCAGUUCA | 926 |
| | 1038 | | GACGUUACC | | | | UAAACCUU | |
| D-3509 | S- | 1391 | GUUUAUGAACUG | 1016 | A-2000 | 1391 | UUAACGUCAGUUC | 916 |
| | 1000 | | AUCUUACCC | | | | AUAAACUU | |
| D-3510 | S- | 1391 | GUUUAUGAACUG | 1019 | A-2000 | 1391 | UUAACGUCAGUUC | 916 |
| | 1003 | | AUCUUAGCC | | | | AUAAACUU | |
| D-3511 | S- | 1391 | GUUUAUGAACUG | 1022 | A-2000 | 1391 | UUAACGUCAGUUC | 916 |
| | 1006 | | AUCUUAACC | | | | AUAAACUU | |
| D-3512 | S- | 1391 | GUUUAUGAACUG | 1025 | A-2000 | 1391 | UUAACGUCAGUUC | 916 |
| | 1009 | | AUCUUAACC | | | | AUAAACUU | |
| D-3513 | S- | 1391 | GUUUAUGAACUG | 1029 | A-2000 | 1391 | UUAACGUCAGUUC | 916 |
| | 1012 | | AUCUUGCCC | | | | AUAAACUU | |
| D-3514 | S- | 1391 | GUUUAUGAACUG | 1030 | A-2000 | 1391 | UUAACGUCAGUUC | 916 |
| | 1013 | | AUCUUGGCC | | | | AUAAACUU | |
| D-3515 | S- | 1391 | GUUUAUGAACUG | 1031 | A-2000 | 1391 | UUAACGUCAGUUC | 916 |
| | 1014 | | AUCUUGACC | | | | AUAAACUU | |
| D-3516 | S- | 1429 | CCACAAUGUUGUG | 1035 | A-2006 | 1428 | UCCGGUCACAACA | 928 |
| | 1018 | | ACUGGCCC | | | | UUGUGGUU | |
| D-3517 | S- | 1429 | CCACAAUGUUGUG | 1042 | A-2006 | 1428 | UCCGGUCACAACA | 928 |
| | 1025 | | ACUGGGCC | | | | UUGUGGUU | |
| D-3518 | S- | 1429 | CCACAAUGUUGUG | 1051 | A-2006 | 1428 | UCCGGUCACAACA | 928 |
| | 1032 | | ACUGGACC | | | | UUGUGGUU | |
| D-3519 | S- | 1429 | CCACAAUGUAGUG | 1060 | A-2006 | 1428 | UCCGGUCACAACA | 928 |
| | 1039 | | ACUGGACC | | | | UUGUGGUU | |
| D-3520 | S- | 2066 | GUGUUAGACGGU | 1017 | A-2001 | 2066 | UGUCGGUACCGUC | 918 |
| | 1001 | | ACUGAUCCC | | | | UAACACUU | |
| D-3521 | S- | 2066 | GUGUUAGACGGU | 1020 | A-2001 | 2066 | UGUCGGUACCGUC | 918 |
| | 1004 | | ACUGAUGCC | | | | UAACACUU | |
| D-3522 | S- | 2066 | GUGUUAGACCGUA | 1023 | A-2001 | 2066 | UGUCGGUACCGUC | 918 |
| | 1007 | | CUGAUACC | | | | UAACACUU | |
| D-3523 | S- | 2066 | GUGUUAGACGGU | 1026 | A-2001 | 2066 | UGUCGGUACCGUC | 918 |
| | 1010 | | ACUGAUACC | | | | UAACACUU | |
| D-3524 | S- | 2079 | CCGACAACCAGUA | 1038 | A-2009 | 2078 | UCCAAAUACUGGU | 934 |
| | 1021 | | UUUGGCCC | | | | UGUCGGUU | |
| D-3525 | S- | 2079 | CCGACAACCAGUA | 1045 | A-2009 | 2078 | UCCAAAUACUGGU | 934 |
| | 1028 | | UUUGGGCC | | | | UGUCGGUU | |
| D-3526 | S- | 2079 | CCGACAACCAGUA | 1055 | A-2009 | 2078 | UCCAAAUACUGGU | 934 |
| | 1035 | | UUUGGACC | | | | UGUCGGUU | |
| D-3527 | S- | 2079 | CCGACAACCUGUA | 1063 | A-2009 | 2078 | UCCAAAUACUGGU | 934 |
| | 1042 | | UUUGGACC | | | | UGUCGGUU | |
| D-3528 | S- | 4544 | GUCACAAAGAACC | 1036 | A-2007 | 4544 | UUGCACGGUUCU | 930 |
| | 1019 | | GUGUACCC | | | | UUGUGACUU | |
| D-3529 | S- | 4544 | GUCACAAAGAACC | 1043 | A-2007 | 4544 | UUGCACGGUUCU | 930 |
| | 1026 | | GUGUAGCC | | | | UUGUGACUU | |
| D-3530 | S- | 4544 | GUCACAAAGAACC | 1052 | A-2007 | 4544 | UUGCACGGUUCU | 930 |
| | 1033 | | GUGUAACC | | | | UUGUGACUU | |
| D-3531 | S- | 4544 | GUCACAAAGUACC | 1061 | A-2007 | 4544 | UUGCACGGUUCU | 930 |
| | 1040 | | GUGUAACC | | | | UUGUGACUU | |
| D-3532 | S- | 4597 | UGAACCUCUUGUU | 1037 | A-2008 | 4597 | UUUUAUAACAAGA | 932 |
| | 1020 | | AUAAACCC | | | | GGUUCAUU | |
| D-3533 | S- | 4597 | UGAACCUCUUGUU | 1044 | A-2008 | 4597 | UUUUAUAACAAGA | 932 |
| | 1027 | | AUAAAGCC | | | | GGUUCAUU | |
| D-3534 | S- | 4597 | UGAACCUCUUGUU | 1053 | A-2008 | 4597 | UUUUAUAACAAGA | 932 |
| | 1034 | | AUAAAACC | | | | GGUUCAUU | |
| D-3535 | S- | 4597 | UGAACCUCUAGUU | 1062 | A-2008 | 4597 | UUUUAUAACAAGA | 932 |
| | 1041 | | AUAAAACC | | | | GGUUCAUU | |
| D-3536 | S- | 4861 | GAUCAUUGGAAU | 1065 | A-2011 | 4860 | UUUAGGAAUUCCA | 938 |
| | 1044 | | UCCUAAUCC | | | | AUGAUCUU | |
| D-3537 | S- | 4861 | GAUCAUUGGAAU | 1067 | A-2011 | 4860 | UUUAGGAAUUCCA | 938 |
| | 1046 | | UCCUAAGCC | | | | AUGAUCUU | |
| D-3538 | S- | 4861 | GAUCAUUGGAAU | 1070 | A-2011 | 4860 | UUUAGGAAUUCCA | 938 |
| | 1048 | | UCCUAAACC | | | | AUGAUCUU | |
| D-3539 | S- | 4861 | GAUCAUUGGUAU | 1073 | A-2011 | 4860 | UUUAGGAAUUCCA | 938 |
| | 1050 | | UCCUAAACC | | | | AUGAUCUU | |
| D-3540 | S- | 4864 | CAUUGGAAUUCCU | 1064 | A-2010 | 4864 | UAUUUUAGGAAU | 936 |
| | 1043 | | AAAAUUCC | | | | UCCAAUGUU | |
| D-3541 | S- | 4864 | CAUUGGAAUUCCU | 1066 | A-2010 | 4864 | UAUUUUAGGAAU | 936 |
| | 1045 | | AAAAUGCC | | | | UCCAAUGUU | |
| D-3542 | S- | 4864 | CAUUGGAAUUCCU | 1068 | A-2010 | 4864 | UAUUUUAGGAAU | 936 |
| | 1047 | | AAAAUACC | | | | UCCAAUGUU | |
| D-3543 | S- | 4864 | CAUUGGAAUACCU | 1072 | A-2010 | 4864 | UAUUUUAGGAAU | 936 |
| | 1049 | | AAAAUACC | | | | UCCAAUGUU | |
| D-3544 | S- | 6188 | GCAAUUCAGUCUC | 1032 | A-2003 | 6188 | UACAACGAGACUG | 922 |
| | 1015 | | GUUGUCCC | | | | AAUUGCUU | |
| D-3545 | S- | 6188 | GCAAUUCAGUCUC | 1039 | A-2003 | 6188 | UACAACGAGACUG | 922 |
| | 1022 | | GUUGUGCC | | | | AAUUGCUU | |
| D-3546 | S- | 6188 | GCAAUUCAGUCUC | 1046 | A-2003 | 6188 | UACAACGAGACUG | 922 |
| | 1029 | | GUUGUACC | | | | AAUUGCUU | |
| D-3547 | S- | 6188 | GCAAUUCAGACUC | 1057 | A-2003 | 6188 | UACAACGAGACUG | 922 |
| | 1036 | | GUUGUACC | | | | AAUUGCUU | |
| D-3548 | S- | 6751 | CCUGCUAGCUCCA | 1018 | A-2002 | 6751 | UAAGCAUGGAGCU | 920 |
| | 1002 | | UGCUUCCC | | | | AGCAGGUU | |
| D-3549 | S- | 6751 | CCUGCUAGCUCCA | 1021 | A-2002 | 6751 | UAAGCAUGGAGCU | 920 |
| | 1005 | | UGCUUGCC | | | | AGCAGGUU | |
| D-3550 | S- | 6751 | CCUGCUAGCACCA | 1024 | A-2002 | 6751 | UAAGCAUGGAGCU | 920 |
| | 1008 | | UGCUUACC | | | | AGCAGGUU | |
| D-3551 | S- | 6751 | CCUGCUAGCUCCA | 1027 | A-2002 | 6751 | UAAGCAUGGAGCU | 920 |
| | 1011 | | UGCUUACC | | | | AGCAGGUU | |

TABLE 5
Sense and antisense strand sequences of HTT dsRNA

| | | | | | | | Anti- | |
|---|---|---|---|---|---|---|---|---|
| | | | Sense | | | | sense | |
| | | | Strand | | | | Strand | |
| siRNA | | | Se- | SS | | | Se- | AS |
| Duplex | SS | Start | quence | SEQ | | Start | quence | SEQ |
| ID | ID | SS | (5′-3′) | ID | AS ID | AS | (5′-3′) | ID |
| D- | S- | 1391 | GUUUAUG | 1074 | A- | 1391 | UUAACGU | 917 |
| 3552 | 1051dt | | AACUGAC | | 2000dt | | CAGUUC | |
| | | | GUUAAdT | | | | AUAAACd | |
| | | | dT | | | | TdT | |
| D- | S- | 2066 | GUGUUAG | 1075 | A- | 2066 | UGUCGGU | 919 |
| 3553 | 1052dt | | ACGGUAC | | 2001dt | | ACCGUC | |
| | | | CGACAdT | | | | UAACACd | |
| | | | dT | | | | TdT | |
| D- | S- | 6751 | CCUGCUA | 1028 | A- | 6751 | UAAGCAU | 921 |
| 3554 | l011dt | | GCUCCAU | | 2002dt | | GGAGCU | |
| | | | CGUUAdT | | | | AGCAGGd | |
| | | | dT | | | | TdT | |
| D- | S- | 1032 | AUAUCAG | 1076 | A- | 1032 | UUAAUCU | 940 |
| 3555 | 1053dt | | UAAAGAG | | 2012dt | | CUUUAC | |
| | | 2 | AUUAAdT | | | 2 | UGAUAUd | |
| | | | dT | | | | TdT | |
| D- | S- | 1386 | UCCAGGU | 1049 | A- | 1386 | UUCAGUU | 925 |
| 3556 | lO3Odt | | UUAUGAA | | 2004dt | | CAUAAAC | |
| | | | CUGAAdT | | | | CUGGAdT | |
| | | | dT | | | | dT | |
| D- | S- | 1390 | GGUUUAU | 1077 | A- | 1390 | UAACGUC | 927 |
| 3557 | 1054dt | | GAACUGA | | 2005dt | | AGUUCA | |
| | | | CGUUAdT | | | | UAAACCd | |
| | | | dT | | | | TdT | |
| D- | S- | 1429 | CCACAAU | 1078 | A- | 1429 | UCCGGUC | 929 |
| 3558 | 1055dt | | GUUGUGA | | 2006dt | | ACAACAU | |
| | | | CCGGAdT | | | | UGUGGdT | |
| | | | dT | | | | dT | |
| D- | S- | 2079 | CCGACAA | 1056 | A- | 2079 | UCCAAAU | 935 |
| 3559 | 1035dt | | CCAGUAU | | 2009dt | | ACUGGU | |
| | | | UUGGAdT | | | | UGUCGGd | |
| | | | dT | | | | TdT | |
| D- | S- | 4544 | GUCACAA | 1079 | A- | 4544 | UUGCACG | 931 |
| 3560 | 1056dt | | AGAACCG | | 2007dt | | GUUCUU | |
| | | | UGCAAdT | | | | UGUGACd | |
| | | | dT | | | | TdT | |
| D- | S- | 4597 | UGAACCU | 1054 | A- | 4597 | UUUUAUA | 933 |
| 3561 | 1034dt | | CUUGUUA | | 2008dt | | ACAAGA | |
| | | | UAAAAdT | | | | GGUUCAd | |
| | | | dT | | | | TdT | |
| D- | S- | 6188 | GCAAUUC | 1047 | A- | 6188 | UACAACG | 923 |
| 3562 | 1029dt | | AGUCUCG | | 2003dt | | AGACUGA | |
| | | | UUGUAdT | | | | AUUGCdT | |
| | | | dT | | | | dT | |
| D- | S- | 4864 | CAUUGGA | 1069 | A- | 4864 | UAUUUUA | 937 |
| 3563 | 1047dt | | AUUCCUA | | 2010dt | | GGAAUU | |
| | | | AAAUAdT | | | | CCAAUGd | |
| | | | dT | | | | TdT | |
| D- | S- | 4861 | GAUCAUU | 1071 | A- | 4861 | UUUAGGA | 939 |
| 3564 | 1048dt | | GGAAUUC | | 2011dt | | AUUCCA | |
| | | | CUAAAdT | | | | AUGAUCd | |
| | | | dT | | | | TdT | |
| D- | S- | 4864 | CAUUGGA | 1080 | A- | 4864 | GAUUUUA | 941 |
| 3565 | 1057dt | | AUUCCUA | | 2013dt | | GGAAUU | |
| | | | AAAUCdT | | | | CCAAUGd | |
| | | | dT | | | | TdT | |

TABLE 6
Antisense and Sense strand
sequences of HTT dsRNA

| | | | Anti- | | | | | |
|---|---|---|---|---|---|---|---|---|
| | | | sense | | | | Sense | |
| | | | Strand | | | | Strand | |
| siRNA | | | Se- | AS | | | Se- | SS |
| Duplex | AS | Start | quence | SEQ | SS | Start | quence | SEQ |
| ID | ID | AS | (5′-3′) | ID | ID | SS | (5′-3′) | ID |
| D-3569 | S- | 6751 | CCUGCU | 1083 | A- | 6751 | UAAGCA | 943 |
| | 1060 | | AGCUCC | | 2015 | | UGGAGC | |
| | | | AUGCUU | | | | UAGCAG | |
| | | | AUU | | | | GGU | |
| D-3570 | S- | 6751 | CCUGCU | 1084 | A- | 6751 | UAAGCA | 920 |
| | 1061 | | AGCUCC | | 2002 | | UGGAGC | |
| | | | AUGCUU | | | | UAGCAG | |
| | | | GUU | | | | GUU | |

In other embodiments, the siRNA molecules of the present invention targeting Htt can be encoded in plasmid vectors, AAV particles, viral genome or other nucleic acid expression vectors for delivery to a cell.

DNA expression plasmids can be used to stably express the siRNA duplexes or dsRNA of the present invention targeting Htt in cells and achieve long-term inhibition of the target gene expression. In one aspect, the sense and antisense strands of a siRNA duplex are typically linked by a short spacer sequence leading to the expression of a stem-loop structure termed short hairpin RNA (shRNA). The hairpin is recognized and cleaved by Dicer, thus generating mature siRNA molecules.

According to the present invention, AAV particles comprising the nucleic acids encoding the siRNA molecules targeting HTT mRNA are produced, the AAV serotypes may be any of the serotypes listed in Table 1. Non-limiting examples of the AAV serotypes include, AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ8, AAV-DJ, AAV-PHP.A, and/or AAV-PHP.B, and variants thereof.

In some embodiments, the siRNA duplexes or encoded dsRNA of the present invention suppress (or degrade) HTT mRNA. Accordingly, the siRNA duplexes or encoded dsRNA can be used to substantially inhibit HTT gene expression in a cell, for example a neuron. In some aspects, the inhibition of HTT gene expression refers to an inhibition by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

According to the present invention, the siRNA molecules are designed and tested for their ability in reducing HTT mRNA levels in cultured cells. Such siRNA molecules may form a duplex such as, but not limited to, include those listed in Table 4, Table 5 or Table 6. As a non-limiting example, the siRNA duplexes may be siRNA duplex IDs: D-3500 to D-3570.

In one embodiment, the siRNA molecules comprise a miRNA seed match for HTT located in the guide strand. In another embodiment, the siRNA molecules comprise a miRNA seed match for HTT located in the passenger strand. In yet another embodiment, the siRNA duplexes or encoded dsRNA targeting HTT gene do not comprise a seed match for HTT located in the guide or passenger strand.

In one embodiment, the siRNA duplexes or encoded dsRNA targeting HTT gene may have almost no significant full-length off target effects for the guide strand. In another embodiment, the siRNA duplexes or encoded dsRNA targeting HTT gene may have almost no significant full-length off target effects for the passenger strand. The siRNA duplexes or encoded dsRNA targeting HTT gene may have less than 1%, 2%, 3%, 4%, 5%, 6%, 7%, 8%, 9%, 10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25% 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off target effects for the passenger strand. In yet another embodiment, the siRNA duplexes or encoded dsRNA targeting HTT gene may have almost no significant full-length off target effects for the guide strand or the passenger strand. The siRNA duplexes or encoded dsRNA targeting HTT gene may have less than 1%, 2%, 3%, 4%, 5%, 6%, 7%, 8%, 9%, 10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25% 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off target effects for the guide or passenger strand.

In one embodiment, the siRNA duplexes or encoded dsRNA targeting HTT gene may have high activity in vitro. In another embodiment, the siRNA molecules may have low activity in vitro. In yet another embodiment, the siRNA duplexes or dsRNA targeting the HTT gene may have high guide strand activity and low passenger strand activity in vitro.

In one embodiment, the siRNA molecules targeting HTT have a high guide strand activity and low passenger strand activity in vitro. The target knock-down (KD) by the guide strand may be at least 40%, 50%, 60%, 65%, 70%, 75%, 80%, 85%, 90%, 95%, 99%, 99.5% or 100%. The target knock-down by the guide strand may be 40-50%, 45-50%, 50-55%, 50-60%, 60-65%, 60-70%, 60-75%, 60-80%, 60-85%, 60-90%, 60-95%, 60-99%, 60-99.5%, 60-100%, 65-70%, 65-75%, 65-80%, 65-85%, 65-90%, 65-95%, 65-99%, 65-99.5%, 65-100%, 70-75%, 70-80%, 70-85%, 70-90%, 70-95%, 70-99%, 70-99.5%, 70-100%, 75-80%, 75-85%, 75-90%, 75-95%, 75-99%, 75-99.5%, 75-100%, 80-85%, 80-90%, 80-95%, 80-99%, 80-99.5%, 80-100%, 85-90%, 85-95%, 85-99%, 85-99.5%, 85-100%, 90-95%, 90-99%, 90-99.5%, 90-100%, 95-99%, 95-99.5%, 95-100%, 99-99.5%, 99-100% or 99.5-100%. As a non-limiting example, the target knock-down (KD) by the guide strand is greater than 70%. As a non-limiting example, the target knock-down (KD) by the guide strand is greater than 60%.

In one embodiment, the siRNA duplex target HTT is designed so there is no miRNA seed match for the sense or antisense sequence to the non-Htt sequence.

In one embodiment, the IC₅₀ of the guide strand in the siRNA duplex targeting HTT for the nearest off target is greater than 100 multiplied by the IC₅₀ of the guide strand for the on-target gene, Htt. As a non-limiting example, if the IC₅₀ of the guide strand for the nearest off target is greater than 100 multiplied by the IC₅₀ of the guide strand for the target then the siRNA molecule is said to have high guide strand selectivity for inhibiting Htt in vitro.

In one embodiment, the 5′ processing of the guide strand of the siRNA duplex targeting HTT has a correct start (n) at the 5′ end at least 75%, 80%, 85%, 90%, 95%, 99% or 100% of the time in vitro or in vivo. As a non-limiting example, the 5′ processing of the guide strand is precise and has a correct start (n) at the 5′ end at least 99% of the time in vitro. As a non-limiting example, the 5′ processing of the guide strand is precise and has a correct start (n) at the 5′ end at least 99% of the time in vivo. As a non-limiting example, the 5′ processing of the guide strand is precise and has a correct start (n) at the 5′ end at least 90% of the time in vitro. As a non-limiting example, the 5′ processing of the guide strand is precise and has a correct start (n) at the 5′ end at least 90% of the time in vivo. As a non-limiting example, the 5′ processing of the guide strand is precise and has a correct start (n) at the 5′ end at least 85% of the time in vitro. As a non-limiting example, the 5′ processing of the guide strand is precise and has a correct start (n) at the 5′ end at least 85% of the time in vivo.

In one embodiment, a passenger-guide strand duplex for HTT is considered effective when the pri- or pre-microRNAs demonstrate, by methods known in the art and described herein, greater than 2-fold guide to passenger strand ratio when processing is measured. As a non-limiting examples, the pri- or pre-microRNAs demonstrate great than 2-fold, 3-fold, 4-fold, 5-fold, 6-fold, 7-fold, 8-fold, 9-fold, 10-fold, 11-fold, 12-fold, 13-fold, 14-fold, 15-fold, or 2 to 5-fold, 2 to 10-fold, 2 to 15-fold, 3 to 5-fold, 3 to 10-fold, 3 to 15-fold, 4 to 5-fold, 4 to 10-fold, 4 to 15-fold, 5 to 10-fold, 5 to 15-fold, 6 to 10-fold, 6 to 15-fold, 7 to 10-fold, 7 to 15-fold, 8 to 10-fold, 8 to 15-fold, 9 to 10-fold, 9 to 15-fold, 10 to 15-fold, 11 to 15-fold, 12 to 15-fold, 13 to 15-fold, or 14 to 15-fold guide to passenger strand ratio when processing is measured.

In one embodiment, the siRNA molecules may be used to silence wild type or mutant HTT by targeting at least one exon on the htt sequence. The exon may be exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, and/or exon 67. As a non-limiting example, the siRNA molecules may be used to silence wild type or mutant HTT by targeting exon 1. As another non-limiting example, the siRNA molecules may be used to silence wild type or mutant HTT by targeting an exon other than exon 1. As another non-limiting example, the siRNA molecules may be used to silence wild type or mutant HTT by targeting exon 50. As another non-limiting example, the siRNA molecules may be used to silence wild type or mutant HTT by targeting exon 67.

In one embodiment, the siRNA molecules may be used to silence wild type and/or mutant HTT by targeting at least one exon on the htt sequence. The exon may be exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, and/or exon 67. As a non-limiting example, the siRNA molecules may be used to silence wild type and/or mutant HTT by targeting exon 1. As another non-limiting example, the siRNA molecules may be used to silence wild type and/or mutant HTT by targeting an exon other than exon 1. As another non-limiting example, the siRNA molecules may be used to silence wild type and/or mutant HTT by targeting exon 50. As another non-limiting example, the siRNA molecules may be used to silence wild type and/or mutant HTT by targeting exon 67.

Design and Sequences of siRNA Duplexes Targeting SOD1 Gene

The present invention provides small interfering RNA (siRNA) duplexes (and modulatory polynucleotides encoding them) that target SOD1 mRNA to interfere with SOD1 gene expression and/or SOD1 protein production.

The encoded siRNA duplex of the present invention contains an antisense strand and a sense strand hybridized together forming a duplex structure, wherein the antisense strand is complementary to the nucleic acid sequence of the targeted SOD1 gene, and wherein the sense strand is homologous to the nucleic acid sequence of the targeted SOD1 gene. In some aspects, the 5′end of the antisense strand has a 5′ phosphate group and the 3′end of the sense strand contains a 3′hydroxyl group. In other aspects, there are none, one or 2 nucleotide overhangs at the 3′end of each strand.

Some guidelines for designing siRNAs have been proposed in the art. These guidelines generally recommend generating a 19-nucleotide duplexed region, symmetric 2-3 nucleotide 3′ overhangs, 5′-phosphate and 3′-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such consideration, together with the specific sequence of a target gene, highly effective siRNA molecules essential for suppressing the SOD1 gene expression may be readily designed.

According to the present invention, siRNA molecules (e.g., siRNA duplexes or encoded dsRNA) that target the SOD1 gene are designed. Such siRNA molecules can specifically, suppress SOD1 gene expression and protein production. In some aspects, the siRNA molecules are designed and used to selectively “knock out” SOD1 gene variants in cells, i.e., mutated SOD1 transcripts that are identified in patients with ALS disease. In some aspects, the siRNA molecules are designed and used to selectively “knock down” SOD1 gene variants in cells. In other aspects, the siRNA molecules are able to inhibit or suppress both the wild type and mutated SOD1 gene.

In one embodiment, an siRNA molecule of the present invention comprises a sense strand and a complementary antisense strand in which both strands are hybridized together to form a duplex structure. The antisense strand has sufficient complementarity to the SOD1 mRNA sequence to direct target-specific RNAi, i.e., the siRNA molecule has a sequence sufficient to trigger the destruction of the target mRNA by the RNAi machinery or process.

In one embodiment, an siRNA molecule of the present invention comprises a sense strand and a complementary antisense strand in which both strands are hybridized together to form a duplex structure and where the start site of the hybridization to the SOD1 mRNA is between nucleotide 15 and 1000 on the SOD1 mRNA sequence. As a non-limiting example, the start site may be between nucleotide 15-25, 15-50, 15-75, 15-100, 100-150, 150-200, 200-250, 250-300, 300-350, 350-400, 400-450, 450-500, 500-550, 550-600, 600-650, 650-700, 700-70, 750-800, 800-850, 850-900, 900-950, and 950-1000 on the SOD1 mRNA sequence. As yet another non-limiting example, the start site may be nucleotide 26, 27, 28, 29, 30, 32, 33, 34, 35, 36, 37, 74, 76, 77, 78, 149, 153, 157, 160, 177, 192, 193, 195, 196, 197, 198, 199, 206, 209, 210, 239, 241, 261, 263, 264, 268, 269, 276, 278, 281, 284, 290, 291, 295, 296, 316, 317, 329, 330, 337, 350, 351, 352, 354, 357, 358, 364, 375, 378, 383, 384, 390, 392, 395, 404, 406, 417, 418, 469, 470, 475, 476, 480, 487, 494, 496, 497, 501, 504, 515, 518, 522, 523, 524, 552, 554, 555, 562, 576, 577, 578, 579, 581, 583, 584, 585, 587, 588, 589, 593, 594, 595, 596, 597, 598, 599, 602, 607, 608, 609, 610, 611, 612, 613, 616, 621, 633, 635, 636, 639, 640, 641, 642, 643, 644, 645, 654, 660, 661, 666, 667, 668, 669, 673, 677, 692, 698, 699, 700, 701, 706, 749, 770, 772, 775, 781, 800, 804, 819, 829, 832, 833, 851, 854, 855, 857, 858, 859, 861, 869, 891, 892, 906, 907, 912, 913, 934, 944, and 947 on the SOD1 mRNA sequence.

In some embodiments, the antisense strand and target SOD1 mRNA sequences have 100% complementarity. The antisense strand may be complementary to any part of the target SOD1 mRNA sequence.

In other embodiments, the antisense strand and target SOD1 mRNA sequences comprise at least one mismatch. As a non-limiting example, the antisense strand and the target SOD1 mRNA sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementarity.

In one embodiment, an siRNA or dsRNA targeting SOD1 includes at least two sequences that are complementary to each other.

According to the present invention, the siRNA molecule targeting SOD1 has a length from about 10-50 or more nucleotides, i.e., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementarity to a target region. In one embodiment, each strand of the siRNA molecule has a length from about 19 to 25, 19 to 24 or 19 to 21 nucleotides. In one embodiment, at least one strand of the siRNA molecule is 19 nucleotides in length. In one embodiment, at least one strand of the siRNA molecule is 20 nucleotides in length. In one embodiment, at least one strand of the siRNA molecule is 21 nucleotides in length. In one embodiment, at least one strand of the siRNA molecule is 22 nucleotides in length. In one embodiment, at least one strand of the siRNA molecule is 23 nucleotides in length. In one embodiment, at least one strand of the siRNA molecule is 24 nucleotides in length. In one embodiment, at least one strand of the siRNA molecule is 25 nucleotides in length.

In some embodiments, the siRNA molecules of the present invention targeting SOD1 can be synthetic RNA duplexes comprising about 19 nucleotides to about 25 nucleotides, and two overhanging nucleotides at the 3′-end. In some aspects, the siRNA molecules may be unmodified RNA molecules. In other aspects, the siRNA molecules may contain at least one modified nucleotide, such as base, sugar or backbone modifications.

In one embodiment, the siRNA molecules of the present invention targeting SOD1 may comprise a nucleotide sequence such as, but not limited to, the antisense (guide) sequences in Table 7 or a fragment or variant thereof. As a non-limiting example, the antisense sequence used in the siRNA molecule of the present invention is at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% of a nucleotide sequence in Table 7. As another non-limiting example, the antisense sequence used in the siRNA molecule of the present invention comprises at least 3, 4, 5, 6, 7, 8, 9, 10, 11, 12, 13, 14, 15, 16, 17, 18, 19, 20, 21 or more than 21 consecutive nucleotides of a nucleotide sequence in Table 7. As yet another non-limiting example, the antisense sequence used in the siRNA molecule of the present invention comprises nucleotides 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 5 to 22, 5 to 21, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 6 to 22, 6 to 21, 6 to 20, 6 to 19, 6 to 18, 6 to 17, 6 to 16, 6 to 15, 6 to 14, 6 to 13, 6 to 12, 6 to 11, 6 to 10, 7 to 22, 7 to 21, 7 to 20, 7 to 19, 7 to 18, 7 to 17, 7 to 16, 7 to 15, 7 to 14, 7 to 13, 7 to 12, 8 to 22, 8 to 21, 8 to 20, 8 to 19, 8 to 18, 8 to 17, 8 to 16, 8 to 15, 8 to 14, 8 to 13, 8 to 12, 9 to 22, 9 to 21, 9 to 20, 9 to 19, 9 to 18, 9 to 17, 9 to 16, 9 to 15, 9 to 14, 10 to 22, 10 to 21, 10 to 20, 10 to 19, 10 to 18, 10 to 17, 10 to 16, 10 to 15, 10 to 14, 11 to 22, 11 to 21, 11 to 20, 11 to 19, 11 to 18, 11 to 17, 11 to 16, 11 to 15, 11 to 14, 12 to 22, 12 to 21, 12 to 20, 12 to 19, 12 to 18, 12 to 17, 12 to 16, 13 to 22, 13 to 21, 13 to 20, 13 to 19, 13 to 18, 13 to 17, 13 to 16, 14 to 22, 14 to 21, 14 to 20, 14 to 19, 14 to 18, 14 to 17, 15 to 22, 15 to 21, 15 to 20, 15 to 19, 15 to 18, 16 to 22, 16 to 21, 16 to 20, 17 to 22, 17 to 21, or 18 to 22 of the sequences in Table 7.

TABLE 7
Antisense Sequences

| Antisense | | SEQ |
|---|---|---|
| ID | Sequence | ID NO |
| A-3000 | UUUAUAGGCCAGACCUCCGdTdT | 1164 |
| A-3001 | UUUUAUAGGCCAGACCUCCdTdT | 1165 |
| A-3002 | UCUUUAUAGGCCAGACCUCdTdT | 1166 |
| A-3003 | UACUUUAUAGGCCAGACCUdTdT | 1167 |
| A-3004 | UUACUUUAUAGGCCAGACCdTdT | 1168 |
| A-3005 | UACUACUUUAUAGGCCAGAdTdT | 1169 |
| A-3006 | UGACUACUUUAUAGGCCAGdTdT | 1170 |
| A-3007 | UCGACUACUUUAUAGGCCAdTdT | 1171 |
| A-3008 | UGCGACUACUUUAUAGGCCdTdT | 1172 |
| A-3009 | UCGCGACUACUUUAUAGGCdTdT | 1173 |
| A-3010 | UCCGCGACUACUUUAUAGGdTdT | 1174 |
| A-3011 | UGCUGCAGGAGACUACGACdTdT | 1175 |
| A-3012 | UACGCUGCAGGAGACUACGdTdT | 1176 |
| A-3013 | UGACGCUGCAGGAGACUACdTdT | 1177 |
| A-3014 | UAGACGCUGCAGGAGACUAdTdT | 1178 |
| A-3015 | UCACGGCCUUCGUCGCCAUdTdT | 1179 |
| A-3016 | UCGCACACGGCCUUCGUCGdTdT | 1180 |
| A-3017 | UAGCACGCACACGGCCUUCdTdT | 1181 |
| A-3018 | UUUCAGCACGCACACGGCCdTdT | 1182 |
| A-3019 | UGCACUGGGCCGUCGCCCUdTdT | 1183 |
| A-3020 | UAAUUGAUGAUGCCCUGCAdTdT | 1184 |
| A-3021 | UAAAUUGAUGAUGCCCUGCdTdT | 1185 |
| A-3022 | UCGAAAUUGAUGAUGCCCUdTdT | 1186 |
| A-3023 | UUCGAAAUUGAUGAUGCCCdTdT | 1187 |
| A-3024 | UCUCGAAAUUGAUGAUGCCdTdT | 1188 |
| A-3025 | UGCUCGAAAUUGAUGAUGCdTdT | 1189 |
| A-3026 | UUGCUCGAAAUUGAUGAUGdTdT | 1190 |
| A-3027 | UUUCCUUCUGCUCGAAAUUdTdT | 1191 |
| A-3028 | UACUUUCCUUCUGCUCGAAdTdT | 1192 |
| A-3029 | UUACUUUCCUUCUGCUCGAdTdT | 1193 |
| A-3030 | UAAUGCUUCCCCACACCUUdTdT | 1194 |
| A-3031 | UUUAAUGCUUCCCCACACCdTdT | 1195 |
| A-3032 | UGCAGGCCUUCAGUCAGUCdTdT | 1196 |
| A-3033 | UAUGCAGGCCUUCAGUCAGdTdT | 1197 |
| A-3034 | UCAUGCAGGCCUUCAGUCAdTdT | 1198 |
| A-3035 | UAAUCCAUGCAGGCCUUCAdTdT | 1199 |
| A-3036 | UGAAUCCAUGCAGGCCUUCdTdT | 1200 |
| A-3037 | UGAACAUGGAAUCCAUGCAdTdT | 1201 |
| A-3038 | UAUGAACAUGGAAUCCAUGdTdT | 1202 |
| A-3039 | UCUCAUGAACAUGGAAUCCdTdT | 1203 |
| A-3040 | UAAACUCAUGAACAUGGAAdTdT | 1204 |
| A-3041 | UAUCUCCAAACUCAUGAACdTdT | 1205 |
| A-3042 | UUAUCUCCAAACUCAUGAAdTdT | 1206 |
| A-3043 | UGUAUUAUCUCCAAACUCAdTdT | 1207 |
| A-3044 | UUGUAUUAUCUCCAAACUCdTdT | 1208 |
| A-3045 | UCCUGCACUGGUACAGCCUdTdT | 1209 |
| A-3046 | UACCUGCACUGGUACAGCCdTdT | 1210 |
| A-3047 | UAUUAAAGUGAGGACCUGCdTdT | 1211 |
| A-3048 | UGAUUAAAGUGAGGACCUGdTdT | 1212 |
| A-3049 | UGAUAGAGGAUUAAAGUGAdTdT | 1213 |
| A-3050 | UACCGUGUUUUCUGGAUAGdTdT | 1214 |
| A-3051 | UCACCGUGUUUUCUGGAUAdTdT | 1215 |
| A-3052 | UCCACCGUGUUUUCUGGAUdTdT | 1216 |
| A-3053 | UGCCCACCGUGUUUUCUGGdTdT | 1217 |
| A-3054 | UUUGGCCCACCGUGUUUUCdTdT | 1218 |
| A-3055 | UUUUGGCCCACCGUGUUUUdTdT | 1219 |
| A-3056 | UUCAUCCUUUGGCCCACCGdTdT | 1220 |
| A-3057 | UCAUGCCUCUCUUCAUCCUdTdT | 1221 |
| A-3058 | UCAACAUGCCUCUCUUCAUdTdT | 1222 |
| A-3059 | UGUCUCCAACAUGCCUCUCdTdT | 1223 |
| A-3060 | UAGUCUCCAACAUGCCUCUdTdT | 1224 |
| A-3061 | UUGCCCAAGUCUCCAACAUdTdT | 1225 |
| A-3062 | UAUUGCCCAAGUCUCCAACdTdT | 1226 |
| A-3063 | UCACAUUGCCCAAGUCUCCdTdT | 1227 |
| A-3064 | UGUCAGCAGUCACAUUGCCdTdT | 1228 |
| A-3065 | UUUGUCAGCAGUCACAUUGdTdT | 1229 |
| A-3066 | UCCACACCAUCUUUGUCAGdTdT | 1230 |
| A-3067 | UGCCACACCAUCUUUGUCAdTdT | 1231 |
| A-3068 | UAUGCAAUGGUCUCCUGAGdTdT | 1232 |
| A-3069 | UGAUGCAAUGGUCUCCUGAdTdT | 1233 |
| A-3070 | UCCAAUGAUGCAAUGGUCUdTdT | 1234 |
| A-3071 | UGCCAAUGAUGCAAUGGUCdTdT | 1235 |
| A-3072 | UUGCGGCCAAUGAUGCAAUdTdT | 1236 |
| A-3073 | UACCAGUGUGCGGCCAAUGdTdT | 1237 |
| A-3074 | UAUGGACCACCAGUGUGCGdTdT | 1238 |
| A-3075 | UUCAUGGACCACCAGUGUGdTdT | 1239 |
| A-3076 | UUUCAUGGACCACCAGUGUdTdT | 1240 |
| A-3077 | UCUUUUUCAUGGACCACCAdTdT | 1241 |
| A-3078 | UCUGCUUUUUCAUGGACCAdTdT | 1242 |
| A-3079 | UGCCCAAGUCAUCUGCUUUdTdT | 1243 |
| A-3080 | UUUUGCCCAAGUCAUCUGCdTdT | 1244 |
| A-3081 | UCACCUUUGCCCAAGUCAUdTdT | 1245 |
| A-3082 | UCCACCUUUGCCCAAGUCAdTdT | 1246 |
| A-3083 | UUCCACCUUUGCCCAAGUCdTdT | 1247 |
| A-3084 | UCGUUUCCUGUCUUUGUACdTdT | 1248 |
| A-3085 | UAGCGUUUCCUGUCUUUGUdTdT | 1249 |
| A-3086 | UCAGCGUUUCCUGUCUUUGdTdT | 1250 |
| A-3087 | UCGACUUCCAGCGUUUCCUdTdT | 1251 |
| A-3088 | UCACCACAAGCCAAACGACdTdT | 1252 |
| A-3089 | UACACCACAAGCCAAACGAdTdT | 1253 |
| A-3090 | UUACACCACAAGCCAAACGdTdT | 1254 |
| A-3091 | UUUACACCACAAGCCAAACdTdT | 1255 |
| A-3092 | UAAUUACACCACAAGCCAAdTdT | 1256 |
| A-3093 | UCCAAUUACACCACAAGCCdTdT | 1257 |
| A-3094 | UCCCAAUUACACCACAAGCdTdT | 1258 |
| A-3095 | UUCCCAAUUACACCACAAGdTdT | 1259 |
| A-3096 | UGAUCCCAAUUACACCACAdTdT | 1260 |
| A-3097 | UCGAUCCCAAUUACACCACdTdT | 1261 |
| A-3098 | UGCGAUCCCAAUUACACCAdTdT | 1262 |
| A-3099 | UUUGGGCGAUCCCAAUUACdTdT | 1263 |
| A-3100 | UAUUGGGCGAUCCCAAUUAdTdT | 1264 |
| A-3101 | UUAUUGGGCGAUCCCAAUUdTdT | 1265 |
| A-3102 | UUUAUUGGGCGAUCCCAAUdTdT | 1266 |
| A-3103 | UUUUAUUGGGCGAUCCCAAdTdT | 1267 |
| A-3104 | UGUUUAUUGGGCGAUCCCAdTdT | 1268 |
| A-3105 | UUGUUUAUUGGGCGAUCCCdTdT | 1269 |
| A-3106 | UGAAUGUUUAUUGGGCGAUdTdT | 1270 |
| A-3107 | UCAAGGGAAUGUUUAUUGGdTdT | 1271 |
| A-3108 | UCCAAGGGAAUGUUUAUUGdTdT | 1272 |
| A-3109 | UUCCAAGGGAAUGUUUAUUdTdT | 1273 |
| A-3110 | UAUCCAAGGGAAUGUUUAUdTdT | 1274 |
| A-3111 | UCAUCCAAGGGAAUGUUUAdTdT | 1275 |
| A-3112 | UACAUCCAAGGGAAUGUUUdTdT | 1276 |
| A-3113 | UUACAUCCAAGGGAAUGUUdTdT | 1277 |
| A-3114 | UGACUACAUCCAAGGGAAUdTdT | 1278 |
| A-3115 | UCCUCAGACUACAUCCAAGdTdT | 1279 |
| A-3116 | UUGAGUUAAGGGGCCUCAGdTdT | 1280 |
| A-3117 | UGAUGAGUUAAGGGGCCUCdTdT | 1281 |
| A-3118 | UAGAUGAGUUAAGGGGCCUdTdT | 1282 |
| A-3119 | UAACAGAUGAGUUAAGGGGdTdT | 1283 |
| A-3120 | UUAACAGAUGAGUUAAGGGdTdT | 1284 |
| A-3121 | UAUAACAGAUGAGUUAAGGdTdT | 1285 |
| A-3122 | UGAUAACAGAUGAGUUAAGdTdT | 1286 |
| A-3123 | UGGAUAACAGAUGAGUUAAdTdT | 1287 |
| A-3124 | UAGGAUAACAGAUGAGUUAdTdT | 1288 |
| A-3125 | UCAGGAUAACAGAUGAGUUdTdT | 1289 |
| A-3126 | UUACAGCUAGCAGGAUAACdTdT | 1290 |
| A-3127 | UCAUUUCUACAGCUAGCAGdTdT | 1291 |
| A-3128 | UACAUUUCUACAGCUAGCAdTdT | 1292 |
| A-3129 | UAGGAUACAUUUCUACAGCdTdT | 1293 |
| A-3130 | UCAGGAUACAUUUCUACAGdTdT | 1294 |
| A-3131 | UUCAGGAUACAUUUCUACAdTdT | 1295 |
| A-3132 | UAUCAGGAUACAUUUCUACdTdT | 1296 |
| A-3133 | UGUUUAUCAGGAUACAUUUdTdT | 1297 |
| A-3134 | UUAAUGUUUAUCAGGAUACdTdT | 1298 |
| A-3135 | UUAAGAUUACAGUGUUUAAdTdT | 1299 |
| A-3136 | UCACUUUUAAGAUUACAGUdTdT | 1300 |
| A-3137 | UACACUUUUAAGAUUACAGdTdT | 1301 |
| A-3138 | UUACACUUUUAAGAUUACAdTdT | 1302 |
| A-3139 | UUUACACUUUUAAGAUUACdTdT | 1303 |
| A-3140 | UCACAAUUACACUUUUAAGdTdT | 1304 |
| A-3141 | UAGUUUCUCACUACAGGUAdTdT | 1305 |
| A-3142 | UUCUUCCAAGUGAUCAUAAdTdT | 1306 |
| A-3143 | UAAUCUUCCAAGUGAUCAUdTdT | 1307 |
| A-3144 | UACAAAUCUUCCAAGUGAUdTdT | 1308 |
| A-3145 | UAACUAUACAAAUCUUCCAdTdT | 1309 |
| A-3146 | UUUUUAACUGAGUUUUAUAdTdT | 1310 |
| A-3147 | UGACAUUUUAACUGAGUUUdTdT | 1311 |
| A-3148 | UCAGGUCAUUGAAACAGACdTdT | 1312 |
| A-3149 | UUGGCAAAAUACAGGUCAUdTdT | 1313 |
| A-3150 | UGUCUGGCAAAAUACAGGUdTdT | 1314 |
| A-3151 | UAGUCUGGCAAAAUACAGGdTdT | 1315 |
| A-3152 | UAUACCCAUCUGUGAUUUAdTdT | 1316 |
| A-3153 | UUUAAUACCCAUCUGUGAUdTdT | 1317 |
| A-3154 | UUUUAAUACCCAUCUGUGAdTdT | 1318 |
| A-3155 | UAGUUUAAUACCCAUCUGUdTdT | 1319 |
| A-3156 | UAAGUUUAAUACCCAUCUGdTdT | 1320 |
| A-3157 | UCAAGUUUAAUACCCAUCUdTdT | 1321 |
| A-3158 | UGACAAGUUUAAUACCCAUdTdT | 1322 |
| A-3159 | UGAAAUUCUGACAAGUUUAdTdT | 1323 |
| A-3160 | UAUUCACAGGCUUGAAUGAdTdT | 1324 |
| A-3161 | UUAUUCACAGGCUUGAAUGdTdT | 1325 |
| A-3162 | UCCAUACAGGGUUUUUAUUdTdT | 1326 |
| A-3163 | UGCCAUACAGGGUUUUUAUdTdT | 1327 |
| A-3164 | UUAAGUGCCAUACAGGGUUdTdT | 1328 |
| A-3165 | UAUAAGUGCCAUACAGGGUdTdT | 1329 |
| A-3166 | UGAUUCUUUUAAUAGCCUCdTdT | 1330 |
| A-3167 | UUUUGAAUUUGGAUUCUUUdTdT | 1331 |
| A-3168 | UUAGUUUGAAUUUGGAUUCdTdT | 1332 |

In one embodiment, the siRNA molecules of the present invention targeting SOD1 may comprise a nucleotide sequence such as, but not limited to, the sense (passenger) sequences in Table 8 or a fragment or variant thereof. As a non-limiting example, the sense sequence used in the siRNA molecule of the present invention is at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% of a nucleotide sequence in Table 8. As another non-limiting example, the sense sequence used in the siRNA molecule of the present invention comprises at least 3, 4, 5, 6, 7, 8, 9, 10, 11, 12, 13, 14, 15, 16, 17, 18, 19, 20, 21 or more than 21 consecutive nucleotides of a nucleotide sequence in Table 8. As yet another non-limiting example, the sense sequence used in the siRNA molecule of the present invention comprises nucleotides 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 5 to 22, 5 to 21, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 6 to 22, 6 to 21, 6 to 20, 6 to 19, 6 to 18, 6 to 17, 6 to 16, 6 to 15, 6 to 14, 6 to 13, 6 to 12, 6 to 11, 6 to 10, 7 to 22, 7 to 21, 7 to 20, 7 to 19, 7 to 18, 7 to 17, 7 to 16, 7 to 15, 7 to 14, 7 to 13, 7 to 12, 8 to 22, 8 to 21, 8 to 20, 8 to 19, 8 to 18, 8 to 17, 8 to 16, 8 to 15, 8 to 14, 8 to 13, 8 to 12, 9 to 22, 9 to 21, 9 to 20, 9 to 19, 9 to 18, 9 to 17, 9 to 16, 9 to 15, 9 to 14, 10 to 22, 10 to 21, 10 to 20, 10 to 19, 10 to 18, 10 to 17, 10 to 16, 10 to 15, 10 to 14, 11 to 22, 11 to 21, 11 to 20, 11 to 19, 11 to 18, 11 to 17, 11 to 16, 11 to 15, 11 to 14, 12 to 22, 12 to 21, 12 to 20, 12 to 19, 12 to 18, 12 to 17, 12 to 16, 13 to 22, 13 to 21, 13 to 20, 13 to 19, 13 to 18, 13 to 17, 13 to 16, 14 to 22, 14 to 21, 14 to 20, 14 to 19, 14 to 18, 14 to 17, 15 to 22, 15 to 21, 15 to 20, 15 to 19, 15 to 18, 16 to 22, 16 to 21, 16 to 20, 17 to 22, 17 to 21, or 18 to 22 of the sequences in Table 8.

TABLE 8
Sense Sequences

| | | SEQ |
|---|---|---|
| Sense | | ID |
| ID | Sequence | NO |
| S-3000 | CGGAGGUCUGGCCUAUAACdTdT | 1333 |
| S-3001 | GGAGGUCUGGCCUAUAAACdTdT | 1334 |
| S-3002 | GAGGUCUGGCCUAUAAAGCdTdT | 1335 |
| S-3003 | AGGUCUGGCCUAUAAAGUCdTdT | 1336 |
| S-3004 | GGUCUGGCCUAUAAAGUACdTdT | 1337 |
| S-3005 | UCUGGCCUAUAAAGUAGUCdTdT | 1338 |
| S-3006 | CUGGCCUAUAAAGUAGUCCdTdT | 1339 |
| S-3007 | UGGCCUAUAAAGUAGUCGCdTdT | 1340 |
| S-3008 | GGCCUAUAAAGUAGUCGCCdTdT | 1341 |
| S-3009 | GCCUAUAAAGUAGUCGCGCdTdT | 1342 |
| S-3010 | CCUAUAAAGUAGUCGCGGCdTdT | 1343 |
| S-3011 | GUCGUAGUCUCCUGCAGCCdTdT | 1344 |
| S-3012 | CGUAGUCUCCUGCAGCGUCdTdT | 1345 |
| S-3013 | GUAGUCUCCUGCAGCGUCCdTdT | 1346 |
| S-3014 | UAGUCUCCUGCAGCGUCUCdTdT | 1347 |
| S-3015 | AUGGCGACGAAGGCCGUGCdTdT | 1348 |
| S-3016 | CGACGAAGGCCGUGUGCGCdTdT | 1349 |
| S-3017 | GAAGGCCGUGUGCGUGCUCdTdT | 1350 |
| S-3018 | GGCCGUGUGCGUGCUGAACdTdT | 1351 |
| S-3019 | AGGGCGACGGCCCAGUGCCdTdT | 1352 |
| S-3020 | UGCAGGGCAUCAUCAAUUCdTdT | 1353 |
| S-3021 | GCAGGGCAUCAUCAAUUUCdTdT | 1354 |
| S-3022 | AGGGCAUCAUCAAUUUCGCdTdT | 1355 |
| S-3023 | GGGCAUCAUCAAUUUCGACdTdT | 1356 |
| S-3024 | GGCAUCAUCAAUUUCGAGCdTdT | 1357 |
| S-3025 | GCAUCAUCAAUUUCGAGCCdTdT | 1358 |
| S-3026 | CAUCAUCAAUUUCGAGCACdTdT | 1359 |
| S-3027 | AAUUUCGAGCAGAAGGAACdTdT | 1360 |
| S-3028 | UUCGAGCAGAAGGAAAGUCdTdT | 1361 |
| S-3029 | UCGAGCAGAAGGAAAGUACdTdT | 1362 |
| S-3030 | AAGGUGUGGGGAAGCAUUCdTdT | 1363 |
| S-3031 | GGUGUGGGGAAGCAUUAACdTdT | 1364 |
| S-3032 | GACUGACUGAAGGCCUGCCdTdT | 1365 |
| S-3033 | CUGACUGAAGGCCUGCAUCdTdT | 1366 |
| S-3034 | UGACUGAAGGCCUGCAUGCdTdT | 1367 |
| S-3035 | UGAAGGCCUGCAUGGAUUCdTdT | 1368 |
| S-3036 | GAAGGCCUGCAUGGAUUCCdTdT | 1369 |
| S-3037 | UGCAUGGAUUCCAUGUUCCdTdT | 1370 |
| S-3038 | CAUGGAUUCCAUGUUCAUCdTdT | 1371 |
| S-3039 | GGAUUCCAUGUUCAUGAGCdTdT | 1372 |
| S-3040 | UUCCAUGUUCAUGAGUUUCdTdT | 1373 |
| S-3041 | GUUCAUGAGUUUGGAGAUCdTdT | 1374 |
| S-3042 | UUCAUGAGUUUGGAGAUACdTdT | 1375 |
| S-3043 | UGAGUUUGGAGAUAAUACCdTdT | 1376 |
| S-3044 | GAGUUUGGAGAUAAUACACdTdT | 1377 |
| S-3045 | AGGCUGUACCAGUGCAGGCdTdT | 1378 |
| S-3046 | GGCUGUACCAGUGCAGGUCdTdT | 1379 |
| S-3047 | GCAGGUCCUCACUUUAAUCdTdT | 1380 |
| S-3048 | CAGGUCCUCACUUUAAUCCdTdT | 1381 |
| S-3049 | UCACUUUAAUCCUCUAUCCdTdT | 1382 |
| S-3050 | CUAUCCAGAAAACACGGUCdTdT | 1383 |
| S-3051 | UAUCCAGAAAACACGGUGCdTdT | 1384 |
| S-3052 | AUCCAGAAAACACGGUGGCdTdT | 1385 |
| S-3053 | CCAGAAAACACGGUGGGCCdTdT | 1386 |
| S-3054 | GAAAACACGGUGGGCCAACdTdT | 1387 |
| S-3055 | AAAACACGGUGGGCCAAACdTdT | 1388 |
| S-3056 | CGGUGGGCCAAAGGAUGACdTdT | 1389 |
| S-3057 | AGGAUGAAGAGAGGCAUGCdTdT | 1390 |
| S-3058 | AUGAAGAGAGGCAUGUUGCdTdT | 1391 |
| S-3059 | GAGAGGCAUGUUGGAGACCdTdT | 1392 |
| S-3060 | AGAGGCAUGUUGGAGACUCdTdT | 1393 |
| S-3061 | AUGUUGGAGACUUGGGCACdTdT | 1394 |
| S-3062 | GUUGGAGACUUGGGCAAUCdTdT | 1395 |
| S-3063 | GGAGACUUGGGCAAUGUGCdTdT | 1396 |
| S-3064 | GGCAAUGUGACUGCUGACCdTdT | 1397 |
| S-3065 | CAAUGUGACUGCUGACAACdTdT | 1398 |
| S-3066 | CUGACAAAGAUGGUGUGGCdTdT | 1399 |
| S-3067 | UGACAAAGAUGGUGUGGCCdTdT | 1400 |
| S-3068 | CUCAGGAGACCAUUGCAUCdTdT | 1401 |
| S-3069 | UCAGGAGACCAUUGCAUCCdTdT | 1402 |
| S-3070 | AGACCAUUGCAUCAUUGGCdTdT | 1403 |
| S-3071 | GACCAUUGCAUCAUUGGCCdTdT | 1404 |
| S-3072 | AUUGCAUCAUUGGCCGCACdTdT | 1405 |
| S-3073 | CAUUGGCCGCACACUGGUCdTdT | 1406 |
| S-3074 | CGCACACUGGUGGUCCAUCdTdT | 1407 |
| S-3075 | CACACUGGUGGUCCAUGACdTdT | 1408 |
| S-3076 | ACACUGGUGGUCCAUGAACdTdT | 1409 |
| S-3077 | UGGUGGUCCAUGAAAAAGCdTdT | 1410 |
| S-3078 | UGGUCCAUGAAAAAGCAGCdTdT | 1411 |
| S-3079 | AAAGCAGAUGACUUGGGCCdTdT | 1412 |
| S-3080 | GCAGAUGACUUGGGCAAACdTdT | 1413 |
| S-3081 | AUGACUUGGGCAAAGGUGCdTdT | 1414 |
| S-3082 | UGACUUGGGCAAAGGUGGCdTdT | 1415 |
| S-3083 | GACUUGGGCAAAGGUGGACdTdT | 1416 |
| S-3084 | GUACAAAGACAGGAAACGCdTdT | 1417 |
| S-3085 | ACAAAGACAGGAAACGCUCdTdT | 1418 |
| S-3086 | CAAAGACAGGAAACGCUGCdTdT | 1419 |
| S-3087 | AGGAAACGCUGGAAGUCGCdTdT | 1420 |
| S-3088 | GUCGUUUGGCUUGUGGUGCdTdT | 1421 |
| S-3089 | UCGUUUGGCUUGUGGUGUCdTdT | 1422 |
| S-3090 | CGUUUGGCUUGUGGUGUACdTdT | 1423 |
| S-3091 | GUUUGGCUUGUGGUGUAACdTdT | 1424 |
| S-3092 | UUGGCUUGUGGUGUAAUUCdTdT | 1425 |
| S-3093 | GGCUUGUGGUGUAAUUGGCdTdT | 1426 |
| S-3094 | GCUUGUGGUGUAAUUGGGCdTdT | 1427 |
| S-3095 | CUUGUGGUGUAAUUGGGACdTdT | 1428 |
| S-3096 | UGUGGUGUAAUUGGGAUCCdTdT | 1429 |
| S-3097 | GUGGUGUAAUUGGGAUCGCdTdT | 1430 |
| S-3098 | UGGUGUAAUUGGGAUCGCCdTdT | 1431 |
| S-3099 | GUAAUUGGGAUCGCCCAACdTdT | 1432 |
| S-3100 | UAAUUGGGAUCGCCCAAUCdTdT | 1433 |
| S-3101 | AAUUGGGAUCGCCCAAUACdTdT | 1434 |
| S-3102 | AUUGGGAUCGCCCAAUAACdTdT | 1435 |
| S-3103 | UUGGGAUCGCCCAAUAAACdTdT | 1436 |
| S-3104 | UGGGAUCGCCCAAUAAACCdTdT | 1437 |
| S-3105 | GGGAUCGCCCAAUAAACACdTdT | 1438 |
| S-3106 | AUCGCCCAAUAAACAUUCCdTdT | 1439 |
| S-3107 | CCAAUAAACAUUCCCUUGCdTdT | 1440 |
| S-3108 | CAAUAAACAUUCCCUUGGCdTdT | 1441 |
| S-3109 | AAUAAACAUUCCCUUGGACdTdT | 1442 |
| S-3110 | AUAAACAUUCCCUUGGAUCdTdT | 1443 |
| S-3111 | UAAACAUUCCCUUGGAUGCdTdT | 1444 |
| S-3112 | AAACAUUCCCUUGGAUGUCdTdT | 1445 |
| S-3113 | AACAUUCCCUUGGAUGUACdTdT | 1446 |
| S-3114 | AUUCCCUUGGAUGUAGUCCdTdT | 1447 |
| S-3115 | CUUGGAUGUAGUCUGAGGCdTdT | 1448 |
| S-3116 | CUGAGGCCCCUUAACUCACdTdT | 1449 |
| S-3117 | GAGGCCCCUUAACUCAUCCdTdT | 1450 |
| S-3118 | AGGCCCCUUAACUCAUCUCdTdT | 1451 |
| S-3119 | CCCCUUAACUCAUCUGUUCdTdT | 1452 |
| S-3120 | CCCUUAACUCAUCUGUUACdTdT | 1453 |
| S-3121 | CCUUAACUCAUCUGUUAUCdTdT | 1454 |
| S-3122 | CUUAACUCAUCUGUUAUCCdTdT | 1455 |
| S-3123 | UUAACUCAUCUGUUAUCCCdTdT | 1456 |
| S-3124 | UAACUCAUCUGUUAUCCUCdTdT | 1457 |
| S-3125 | AACUCAUCUGUUAUCCUGCdTdT | 1458 |
| S-3126 | GUUAUCCUGCUAGCUGUACdTdT | 1459 |
| S-3127 | CUGCUAGCUGUAGAAAUGCdTdT | 1460 |
| S-3128 | UGCUAGCUGUAGAAAUGUCdTdT | 1461 |
| S-3129 | GCUGUAGAAAUGUAUCCUCdTdT | 1462 |
| S-3130 | CUGUAGAAAUGUAUCCUGCdTdT | 1463 |
| S-3131 | UGUAGAAAUGUAUCCUGACdTdT | 1464 |
| S-3132 | GUAGAAAUGUAUCCUGAUCdTdT | 1465 |
| S-3133 | AAAUGUAUCCUGAUAAACCdTdT | 1466 |
| S-3134 | GUAUCCUGAUAAACAUUACdTdT | 1467 |
| S-3135 | UUAAACACUGUAAUCUUACdTdT | 1468 |
| S-3136 | ACUGUAAUCUUAAAAGUGCdTdT | 1469 |
| S-3137 | CUGUAAUCUUAAAAGUGUCdTdT | 1470 |
| S-3138 | UGUAAUCUUAAAAGUGUACdTdT | 1471 |
| S-3139 | GUAAUCUUAAAAGUGUAACdTdT | 1472 |
| S-3140 | CUUAAAAGUGUAAUUGUGCdTdT | 1473 |
| S-3141 | UACCUGUAGUGAGAAACUCdTdT | 1474 |
| S-3142 | UUAUGAUCACUUGGAAGACdTdT | 1475 |
| S-3143 | AUGAUCACUUGGAAGAUUCdTdT | 1476 |
| S-3144 | AUCACUUGGAAGAUUUGUCdTdT | 1477 |
| S-3145 | UGGAAGAUUUGUAUAGUUCdTdT | 1478 |
| S-3146 | UAUAAAACUCAGUUAAAACdTdT | 1479 |
| S-3147 | AAACUCAGUUAAAAUGUCCdTdT | 1480 |
| S-3148 | GUCUGUUUCAAUGACCUGCdTdT | 1481 |
| S-3149 | AUGACCUGUAUUUUGCCACdTdT | 1482 |
| S-3150 | ACCUGUAUUUUGCCAGACCdTdT | 1483 |
| S-3151 | CCUGUAUUUUGCCAGACUCdTdT | 1484 |
| S-3152 | UAAAUCACAGAUGGGUAUCdTdT | 1485 |
| S-3153 | AUCACAGAUGGGUAUUAACdTdT | 1486 |
| S-3154 | UCACAGAUGGGUAUUAAACdTdT | 1487 |
| S-3155 | ACAGAUGGGUAUUAAACUCdTdT | 1488 |
| S-3156 | CAGAUGGGUAUUAAACUUCdTdT | 1489 |
| S-3157 | AGAUGGGUAUUAAACUUGCdTdT | 1490 |
| S-3158 | AUGGGUAUUAAACUUGUCCdTdT | 1491 |
| S-3159 | UAAACUUGUCAGAAUUUCCdTdT | 1492 |
| S-3160 | UCAUUCAAGCCUGUGAAUCdTdT | 1493 |
| S-3161 | CAUUCAAGCCUGUGAAUACdTdT | 1494 |
| S-3162 | AAUAAAAACCCUGUAUGGCdTdT | 1495 |
| S-3163 | AUAAAAACCCUGUAUGGCCdTdT | 1496 |
| S-3164 | AACCCUGUAUGGCACUUACdTdT | 1497 |
| S-3165 | ACCCUGUAUGGCACUUAUCdTdT | 1498 |
| S-3166 | GAGGCUAUUAAAAGAAUCCdTdT | 1499 |
| S-3167 | AAAGAAUCCAAAUUCAAACdTdT | 1500 |
| S-3168 | GAAUCCAAAUUCAAACUACdTdT | 1501 |

In one embodiment, the siRNA molecules of the present invention targeting SOD1 may comprise an antisense sequence from Table 7 and a sense sequence from Table 8, or a fragment or variant thereof. As a non-limiting example, the antisense sequence and the sense sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementarity.

In one embodiment, the siRNA molecules of the present invention targeting SOD1 may comprise the sense and antisense siRNA duplex as described in Table 9. As a non-limiting example, these siRNA duplexes may be tested for in vitro inhibitory activity on endogenous SOD1 gene expression. The start site for the sense and antisense sequence is compared to SOD1 gene sequence known as NM_000454.4 (SEQ ID NO: 1502) from NCBI.

TABLE 9
Sense and antisense strand sequences of SOD1 dsRNA

| | | Sense | | | Antisense | |
|---|---|---|---|---|---|---|
| siRNA | | Strand | | | Strand | |
| Duplex | | Sequence | SS | | Sequence | AS |
| ID | SS ID | (5′-3′) | SEQ ID | AS ID | (5′-3′) | SEQ ID |
| D-2741 | S-3000 | CGGAGGUCUGGCCU | 1333 | A-3000 | UUUAUAGGCCAGA | 1164 |
| | | AUAACdTdT | | | CCUCCGdTdT | |
| D-2742 | S-3001 | GGAGGUCUGGCCUA | 1334 | A-3001 | UUUUAUAGGCCAG | 1165 |
| | | UAAACdTdT | | | ACCUCCdTdT | |
| D-2743 | S-3002 | GAGGUCUGGCCUAU | 1335 | A-3002 | UCUUUAUAGGCCA | 1166 |
| | | AAAGCdTdT | | | GACCUCdTdT | |
| D-2744 | S-3003 | AGGUCUGGCCUAUA | 1336 | A-3003 | UACUUUAUAGGCC | 1167 |
| | | AAGUCdTdT | | | AGACCUdTdT | |
| D-2745 | S-3004 | GGUCUGGCCUAUAA | 1337 | A-3004 | UUACUUUAUAGGC | 1168 |
| | | AGUACdTdT | | | CAGACCdTdT | |
| D-2746 | S-3005 | UCUGGCCUAUAAAG | 1338 | A-3005 | UACUACUUUAUAG | 1169 |
| | | UAGUCdTdT | | | GCCAGAdTdT | |
| D-2747 | S-3006 | CUGGCCUAUAAAGU | 1339 | A-3006 | UGACUACUUUAUA | 1170 |
| | | AGUCCdTdT | | | GGCCAGdTdT | |
| D-2748 | S-3007 | UGGCCUAUAAAGUA | 1340 | A-3007 | UCGACUACUUUAU | 1171 |
| | | GUCGCdTdT | | | AGGCCAdTdT | |
| D-2749 | S-3008 | GGCCUAUAAAGUAG | 1341 | A-3008 | UGCGACUACUUUA | 1172 |
| | | UCGCCdTdT | | | UAGGCCdTdT | |
| D-2750 | S-3009 | GCCUAUAAAGUAGU | 1342 | A-3009 | UCGCGACUACUUU | 1173 |
| | | CGCGCdTdT | | | AUAGGCdTdT | |
| D-2751 | S-3010 | CCUAUAAAGUAGUC | 1343 | A-3010 | UCCGCGACUACUU | 1174 |
| | | GCGGCdTdT | | | UAUAGGdTdT | |
| D-2752 | S-3011 | GUCGUAGUCUCCUG | 1344 | A-3011 | UGCUGCAGGAGAC | 1175 |
| | | CAGCCdTdT | | | UACGACdTdT | |
| D-2753 | S-3012 | CGUAGUCUCCUGCA | 1345 | A-3012 | UACGCUGCAGGAG | 1176 |
| | | GCGUCdTdT | | | ACUACGdTdT | |
| D-2754 | S-3013 | GUAGUCUCCUGCAG | 1346 | A-3013 | UGACGCUGCAGGA | 1177 |
| | | CGUCCdTdT | | | GACUACdTdT | |
| D-2755 | S-3014 | UAGUCUCCUGCAGC | 1347 | A-3014 | UAGACGCUGCAGG | 1178 |
| | | GUCUCdTdT | | | AGACUAdTdT | |
| D-2756 | S-3015 | AUGGCGACGAAGGC | 1348 | A-3015 | UCACGGCCUUCGU | 1179 |
| | | CGUGCdTdT | | | CGCCAUdTdT | |
| D-2757 | S-3016 | CGACGAAGGCCGUG | 1349 | A-3016 | UCGCACACGGCCU | 1180 |
| | | UGCGCdTdT | | | UCGUCGdTdT | |
| D-2758 | S-3017 | GAAGGCCGUGUGCG | 1350 | A-3017 | UAGCACGCACACG | 1181 |
| | | UGCUCdTdT | | | GCCUUCdTdT | |
| D-2759 | S-3018 | GGCCGUGUGCGUGC | 1351 | A-3018 | UUUCAGCACGCAC | 1182 |
| | | UGAACdTdT | | | ACGGCCdTdT | |
| D-2760 | S-3019 | AGGGCGACGGCCCA | 1352 | A-3019 | UGCACUGGGCCGU | 1183 |
| | | GUGCCdTdT | | | CGCCCUdTdT | |
| D-2761 | S-3020 | UGCAGGGCAUCAUC | 1353 | A-3020 | UAAUUGAUGAUGC | 1184 |
| | | AAUUCdTdT | | | CCUGCAdTdT | |
| D-2762 | S-3021 | GCAGGGCAUCAUCA | 1354 | A-3021 | UAAAUUGAUGAUG | 1185 |
| | | AUUUCdTdT | | | CCCUGCdTdT | |
| D-2763 | S-3022 | AGGGCAUCAUCAAU | 1355 | A-3022 | UCGAAAUUGAUGA | 1186 |
| | | UUCGCdTdT | | | UGCCCUdTdT | |
| D-2764 | S-3023 | GGGCAUCAUCAAUU | 1356 | A-3023 | UUCGAAAUUGAUG | 1187 |
| | | UCGACdTdT | | | AUGCCCdTdT | |
| D-2765 | S-3024 | GGCAUCAUCAAUUU | 1357 | A-3024 | UCUCGAAAUUGAU | 1188 |
| | | CGAGCdTdT | | | GAUGCCdTdT | |
| D-2766 | S-3025 | GCAUCAUCAAUUUC | 1358 | A-3025 | UGCUCGAAAUUGA | 1189 |
| | | GAGCCdTdT | | | UGAUGCdTdT | |
| D-2767 | S-3026 | CAUCAUCAAUUUCG | 1359 | A-3026 | UUGCUCGAAAUUG | 1190 |
| | | AGCACdTdT | | | AUGAUGdTdT | |
| D-2768 | S-3027 | AAUUUCGAGCAGAA | 1360 | A-3027 | UUUCCUUCUGCUC | 1191 |
| | | GGAACdTdT | | | GAAAUUdTdT | |
| D-2769 | S-3028 | UUCGAGCAGAAGGA | 1361 | A-3028 | UACUUUCCUUCUG | 1192 |
| | | AAGUCdTdT | | | CUCGAAdTdT | |
| D-2770 | S-3029 | UCGAGCAGAAGGAA | 1362 | A-3029 | UUACUUUCCUUCU | 1193 |
| | | AGUACdTdT | | | GCUCGAdTdT | |
| D-2771 | S-3030 | AAGGUGUGGGGAAG | 1363 | A-3030 | UAAUGCUUCCCCA | 1194 |
| | | CAUUCdTdT | | | CACCUUdTdT | |
| D-2772 | S-3031 | GGUGUGGGGAAGCA | 1364 | A-3031 | UUUAAUGCUUCCC | 1195 |
| | | UUAACdTdT | | | CACACCdTdT | |
| D-2773 | S-3032 | GACUGACUGAAGGC | 1365 | A-3032 | UGCAGGCCUUCAG | 1196 |
| | | CUGCCdTdT | | | UCAGUCdTdT | |
| D-2774 | S-3033 | CUGACUGAAGGCCU | 1366 | A-3033 | UAUGCAGGCCUUC | 1197 |
| | | GCAUCdTdT | | | AGUCAGdTdT | |
| D-2775 | S-3034 | UGACUGAAGGCCUG | 1367 | A-3034 | UCAUGCAGGCCUU | 1198 |
| | | CAUGCdTdT | | | CAGUCAdTdT | |
| D-2776 | S-3035 | UGAAGGCCUGCAUG | 1368 | A-3035 | UAAUCCAUGCAGG | 1199 |
| | | GAUUCdTdT | | | CCUUCAdTdT | |
| D-2777 | S-3036 | GAAGGCCUGCAUGG | 1369 | A-3036 | UGAAUCCAUGCAG | 1200 |
| | | AUUCCdTdT | | | GCCUUCdTdT | |
| D-2778 | S-3037 | UGCAUGGAUUCCAU | 1370 | A-3037 | UGAACAUGGAAUC | 1201 |
| | | GUUCCdTdT | | | CAUGCAdTdT | |
| D-2779 | S-3038 | CAUGGAUUCCAUGU | 1371 | A-3038 | UAUGAACAUGGAA | 1202 |
| | | UCAUCdTdT | | | UCCAUGdTdT | |
| D-2780 | S-3039 | GGAUUCCAUGUUCA | 1372 | A-3039 | UCUCAUGAACAUG | 1203 |
| | | UGAGCdTdT | | | GAAUCCdTdT | |
| D-2781 | S-3040 | UUCCAUGUUCAUGA | 1373 | A-3040 | UAAACUCAUGAAC | 1204 |
| | | GUUUCdTdT | | | AUGGAAdTdT | |
| D-2782 | S-3041 | GUUCAUGAGUUUGG | 1374 | A-3041 | UAUCUCCAAACUC | 1205 |
| | | AGAUCdTdT | | | AUGAACdTdT | |
| D-2783 | S-3042 | UUCAUGAGUUUGGA | 1375 | A-3042 | UUAUCUCCAAACU | 1206 |
| | | GAUACdTdT | | | CAUGAAdTdT | |
| D-2784 | S-3043 | UGAGUUUGGAGAUA | 1376 | A-3043 | UGUAUUAUCUCCA | 1207 |
| | | AUACCdTdT | | | AACUCAdTdT | |
| D-2785 | S-3044 | GAGUUUGGAGAUAA | 1377 | A-3044 | UUGUAUUAUCUCC | 1208 |
| | | UACACdTdT | | | AAACUCdTdT | |
| D-2786 | S-3045 | AGGCUGUACCAGUG | 1378 | A-3045 | UCCUGCACUGGUA | 1209 |
| | | CAGGCdTdT | | | CAGCCUdTdT | |
| D-2787 | S-3046 | GGCUGUACCAGUGC | 1379 | A-3046 | UACCUGCACUGGU | 1210 |
| | | AGGUCdTdT | | | ACAGCCdTdT | |
| D-2788 | S-3047 | GCAGGUCCUCACUU | 1380 | A-3047 | UAUUAAAGUGAGG | 1211 |
| | | UAAUCdTdT | | | ACCUGCdTdT | |
| D-2789 | S-3048 | CAGGUCCUCACUUU | 1381 | A-3048 | UGAUUAAAGUGAG | 1212 |
| | | AAUCCdTdT | | | GACCUGdTdT | |
| D-2790 | S-3049 | UCACUUUAAUCCUC | 1382 | A-3049 | UGAUAGAGGAUUA | 1213 |
| | | UAUCCdTdT | | | AAGUGAdTdT | |
| D-2791 | S-3050 | CUAUCCAGAAAACA | 1383 | A-3050 | UACCGUGUUUUCU | 1214 |
| | | CGGUCdTdT | | | GGAUAGdTdT | |
| D-2792 | S-3051 | UAUCCAGAAAACAC | 1384 | A-3051 | UCACCGUGUUUUC | 1215 |
| | | GGUGCdTdT | | | UGGAUAdTdT | |
| D-2793 | S-3052 | AUCCAGAAAACACG | 1385 | A-3052 | UCCACCGUGUUUU | 1216 |
| | | GUGGCdTdT | | | CUGGAUdTdT | |
| D-2794 | S-3053 | CCAGAAAACACGGU | 1386 | A-3053 | UGCCCACCGUGUU | 1217 |
| | | GGGCCdTdT | | | UUCUGGdTdT | |
| D-2795 | S-3054 | GAAAACACGGUGGG | 1387 | A-3054 | UUUGGCCCACCGU | 1218 |
| | | CCAACdTdT | | | GUUUUCdTdT | |
| D-2796 | S-3055 | AAAACACGGUGGGC | 1388 | A-3055 | UUUUGGCCCACCG | 1219 |
| | | CAAACdTdT | | | UGUUUUdTdT | |
| D-2797 | S-3056 | CGGUGGGCCAAAGG | 1389 | A-3056 | UUCAUCCUUUGGC | 1220 |
| | | AUGACdTdT | | | CCACCGdTdT | |
| D-2798 | S-3057 | AGGAUGAAGAGAGG | 1390 | A-3057 | UCAUGCCUCUCUU | 1221 |
| | | CAUGCdTdT | | | CAUCCUdTdT | |
| D-2799 | S-3058 | AUGAAGAGAGGCAU | 1391 | A-3058 | UCAACAUGCCUCU | 1222 |
| | | GUUGCdTdT | | | CUUCAUdTdT | |
| D-2800 | S-3059 | GAGAGGCAUGUUGG | 1392 | A-3059 | UGUCUCCAACAUG | 1223 |
| | | AGACCdTdT | | | CCUCUCdTdT | |
| D-2801 | S-3060 | AGAGGCAUGUUGGA | 1393 | A-3060 | UAGUCUCCAACAU | 1224 |
| | | GACUCdTdT | | | GCCUCUdTdT | |
| D-2802 | S-3061 | AUGUUGGAGACUUG | 1394 | A-3061 | UUGCCCAAGUCUC | 1225 |
| | | GGCACdTdT | | | CAACAUdTdT | |
| D-2803 | S-3062 | GUUGGAGACUUGGG | 1395 | A-3062 | UAUUGCCCAAGUC | 1226 |
| | | CAAUCdTdT | | | UCCAACdTdT | |
| D-2804 | S-3063 | GGAGACUUGGGCAA | 1396 | A-3063 | UCACAUUGCCCAA | 1227 |
| | | UGUGCdTdT | | | GUCUCCdTdT | |
| D-2805 | S-3064 | GGCAAUGUGACUGC | 1397 | A-3064 | UGUCAGCAGUCAC | 1228 |
| | | UGACCdTdT | | | AUUGCCdTdT | |
| D-2806 | S-3065 | CAAUGUGACUGCUG | 1398 | A-3065 | UUUGUCAGCAGUC | 1229 |
| | | ACAACdTdT | | | ACAUUGdTdT | |
| D-2807 | S-3066 | CUGACAAAGAUGGU | 1399 | A-3066 | UCCACACCAUCUU | 1230 |
| | | G UGGCdTdT | | | UGUCAGdTdT | |
| D-2808 | S-3067 | UGACAAAGAUGGUG | 1400 | A-3067 | UGCCACACCAUCU | 1231 |
| | | UGGCCdTdT | | | UUGUCAdTdT | |
| D-2809 | S-3068 | CUCAGGAGACCAUU | 1401 | A-3068 | UAUGCAAUGGUCU | 1232 |
| | | GCAUCdTdT | | | CCUGAGdTdT | |
| D-2810 | S-3069 | UCAGGAGACCAUUG | 1402 | A-3069 | UGAUGCAAUGGUC | 1233 |
| | | CAUCCdTdT | | | UCCUGAdTdT | |
| D-2811 | S-3070 | AGACCAUUGCAUCA | 1403 | A-3070 | UCCAAUGAUGCAA | 1234 |
| | | UUGGCdTdT | | | UGGUCUdTdT | |
| D-2812 | S-3071 | GACCAUUGCAUCAU | 1404 | A-3071 | UGCCAAUGAUGCA | 1235 |
| | | UGGCCdTdT | | | AUGGUCdTdT | |
| D-2813 | S-3072 | AUUGCAUCAUUGGC | 1405 | A-3072 | UUGCGGCCAAUGA | 1236 |
| | | CGCACdTdT | | | UGCAAUdTdT | |
| D-2814 | S-3073 | CAUUGGCCGCACAC | 1406 | A-3073 | UACCAGUGUGCGG | 1237 |
| | | UGGUCdTdT | | | CCAAUGdTdT | |
| D-2815 | S-3074 | CGCACACUGGUGGU | 1407 | A-3074 | UAUGGACCACCAG | 1238 |
| | | CCAUCdTdT | | | UGUGCGdTdT | |
| D-2816 | S-3075 | CACACUGGUGGUCC | 1408 | A-3075 | UUCAUGGACCACC | 1239 |
| | | AUGACdTdT | | | AGUGUGdTdT | |
| D-2817 | S-3076 | ACACUGGUGGUCCA | 1409 | A-3076 | UUUCAUGGACCAC | 1240 |
| | | UGAACdTdT | | | CAGUGUdTdT | |
| D-2818 | S-3077 | UGGUGGUCCAUGAA | 1410 | A-3077 | UCUUUUUCAUGGA | 1241 |
| | | AAAGCdTdT | | | CCACCAdTdT | |
| D-2819 | S-3078 | UGGUCCAUGAAAAA | 1411 | A-3078 | UCUGCUUUUUCAU | 1242 |
| | | GCAGCdTdT | | | GGACCAdTdT | |
| D-2820 | S-3079 | AAAGCAGAUGACUU | 1412 | A-3079 | UGCCCAAGUCAUC | 1243 |
| | | GGGCCdTdT | | | UGCUUUdTdT | |
| D-2821 | S-3080 | GCAGAUGACUUGGG | 1413 | A-3080 | UUUUGCCCAAGUC | 1244 |
| | | CAAACdTdT | | | AUCUGCdTdT | |
| D-2822 | S-3081 | AUGACUUGGGCAAA | 1414 | A-3081 | UCACCUUUGCCCA | 1245 |
| | | GGUGCdTdT | | | AGUCAUdTdT | |
| D-2823 | S-3082 | UGACUUGGGCAAAG | 1415 | A-3082 | UCCACCUUUGCCC | 1246 |
| | | GUGGCdTdT | | | AAGUCAdTdT | |
| D-2824 | S-3083 | GACUUGGGCAAAGG | 1416 | A-3083 | UUCCACCUUUGCC | 1247 |
| | | UGGACdTdT | | | CAAGUCdTdT | |
| D-2825 | S-3084 | GUACAAAGACAGGA | 1417 | A-3084 | UCGUUUCCUGUCU | 1248 |
| | | AACGCdTdT | | | UUGUACdTdT | |
| D-2826 | S-3085 | ACAAAGACAGGAAA | 1418 | A-3085 | UAGCGUUUCCUGU | 1249 |
| | | CGCUCdTdT | | | CUUUGUdTdT | |
| D-2827 | S-3086 | CAAAGACAGGAAAC | 1419 | A-3086 | UCAGCGUUUCCUG | 1250 |
| | | GCUGCdTdT | | | UCUUUGdTdT | |
| D-2828 | S-3087 | AGGAAACGCUGGAA | 1420 | A-3087 | UCGACUUCCAGCG | 1251 |
| | | GUCGCdTdT | | | UUUCCUdTdT | |
| D-2829 | S-3088 | GUCGUUUGGCUUGU | 1421 | A-3088 | UCACCACAAGCCA | 1252 |
| | | GGUGCdTdT | | | AACGACdTdT | |
| D-2830 | S-3089 | UCGUUUGGCUUGUG | 1422 | A-3089 | UACACCACAAGCC | 1253 |
| | | GUGUCdTdT | | | AAACGAdTdT | |
| D-2831 | S-3090 | CGUUUGGCUUGUGG | 1423 | A-3090 | UUACACCACAAGC | 1254 |
| | | UGUACdTdT | | | CAAACGdTdT | |
| D-2832 | S-3091 | GUUUGGCUUGUGGU | 1424 | A-3091 | UUUACACCACAAG | 1255 |
| | | GUAACdTdT | | | CCAAACdTdT | |
| D-2833 | S-3092 | UUGGCUUGUGGUGU | 1425 | A-3092 | UAAUUACACCACA | 1256 |
| | | AAUUCdTdT | | | AGCCAAdTdT | |
| D-2834 | S-3093 | GGCUUGUGGUGUAA | 1426 | A-3093 | UCCAAUUACACCA | 1257 |
| | | UUGGCdTdT | | | CAAGCCdTdT | |
| D-2835 | S-3094 | GCUUGUGGUGUAAU | 1427 | A-3094 | UCCCAAUUACACC | 1258 |
| | | UGGGCdTdT | | | ACAAGCdTdT | |
| D-2836 | S-3095 | CUUGUGGUGUAAUU | 1428 | A-3095 | UUCCCAAUUACAC | 1259 |
| | | GGGACdTdT | | | CACAAGdTdT | |
| D-2837 | S-3096 | UGUGGUGUAAUUGG | 1429 | A-3096 | UGAUCCCAAUUAC | 1260 |
| | | GAUCCdTdT | | | ACCACAdTdT | |
| D-2838 | S-3097 | GUGGUGUAAUUGGG | 1430 | A-3097 | UCGAUCCCAAUUA | 1261 |
| | | AUCGCdTdT | | | ACCCACdTdT | |
| D-2839 | S-3098 | UGGUGUAAUUGGGA | 1431 | A-3098 | UGCGAUCCCAAUU | 1262 |
| | | UCGCCdTdT | | | ACACCAdTdT | |
| D-2840 | S-3099 | GUAAUUGGGAUCGC | 1432 | A-3099 | UUUGGGCGAUCCC | 1263 |
| | | CCAACdTdT | | | AAUUACdTdT | |
| D-2841 | S-3100 | UAAUUGGGAUCGCC | 1433 | A-3100 | UAUUGGGCGAUCC | 1264 |
| | | CAAUCdTdT | | | CAAUUAdTdT | |
| D-2842 | S-3101 | AAUUGGGAUCGCCC | 1434 | A-3101 | UUAUUGGGCGAUC | 1265 |
| | | AAUACdTdT | | | CCAAUUdTdT | |
| D-2843 | S-3102 | AUUGGGAUCGCCCA | 1435 | A-3102 | UUUAUUGGGCGAU | 1266 |
| | | AUAACdTdT | | | CCCAAUdTdT | |
| D-2844 | S-3103 | UUGGGAUCGCCCAA | 1436 | A-3103 | UUUUAUUGGGCGA | 1267 |
| | | UAAACdTdT | | | UCCCAAdTdT | |
| D-2845 | S-3104 | UGGGAUCGCCCAAU | 1437 | A-3104 | UGUUUAUUGGGCG | 1268 |
| | | AAACCdTdT | | | AUCCCAdTdT | |
| D-2846 | S-3105 | GGGAUCGCCCAAUA | 1438 | A-3105 | UUGUUUAUUGGGC | 1269 |
| | | AACACdTdT | | | GAUCCCdTdT | |
| D-2847 | S-3106 | AUCGCCCAAUAAAC | 1439 | A-3106 | UGAAUGUUUAUUG | 1270 |
| | | AUUCCdTdT | | | GGCGAUdTdT | |
| D-2848 | S-3107 | CCAAUAAACAUUCC | 1440 | A-3107 | UCAAGGGAAUGUU | 1271 |
| | | CUUGCdTdT | | | UAUUGGdTdT | |
| D-2849 | S-3108 | CAAUAAACAUUCCC | 1441 | A-3108 | UCCAAGGGAAUGU | 1272 |
| | | UUGGCdTdT | | | UUAUUGdTdT | |
| D-2850 | S-3109 | AAUAAACAUUCCCU | 1442 | A-3109 | UUCCAAGGGAAUG | 1273 |
| | | UGGACdTdT | | | UUUAUUdTdT | |
| D-2851 | S-3110 | AUAAACAUUCCCUU | 1443 | A-3110 | UAUCCAAGGGAAU | 1274 |
| | | GGAUCdTdT | | | GUUUAUdTdT | |
| D-2852 | S-3111 | UAAACAUUCCCUUG | 1444 | A-3111 | UCAUCCAAGGGAA | 1275 |
| | | GAUGCdTdT | | | UGUUUAdTdT | |
| D-2853 | S-3112 | AAACAUUCCCUUGG | 1445 | A-3112 | UACAUCCAAGGGA | 1276 |
| | | AUGUCdTdT | | | AUGUUUdTdT | |
| D-2854 | S-3113 | AACAUUCCCUUGGA | 1446 | A-3113 | UUACAUCCAAGGG | 1277 |
| | | UGUACdTdT | | | AAUGUUdTdT | |
| D-2855 | S-3114 | AUUCCCUUGGAUGU | 1447 | A-3114 | UGACUACAUCCAA | 1278 |
| | | AGUCCdTdT | | | GGGAAUdTdT | |
| D-2856 | S-3115 | CUUGGAUGUAGUCU | 1448 | A-3115 | UCCUCAGACUACA | 1279 |
| | | GAGGCdTdT | | | UCCAAGdTdT | |
| D-2857 | S-3116 | CUGAGGCCCCUUAA | 1449 | A-3116 | UUGAGUUAAGGGG | 1280 |
| | | CUCACdTdT | | | CCUCAGdTdT | |
| D-2858 | S-3117 | GAGGCCCCUUAACU | 1450 | A-3117 | UGAUGAGUUAAGG | 1281 |
| | | CAUCCdTdT | | | GGCCUCdTdT | |
| D-2859 | S-3118 | AGGCCCCUUAACUC | 1451 | A-3118 | UAGAUGAGUUAAG | 1282 |
| | | AUCUCdTdT | | | GGGCCUdTdT | |
| D-2860 | S-3119 | CCCCUUAACUCAUC | 1452 | A-3119 | UAACAGAUGAGUU | 1283 |
| | | UGUUCdTdT | | | AAGGGGdTdT | |
| D-2861 | S-3120 | CCCUUAACUCAUCU | 1453 | A-3120 | UUAACAGAUGAGU | 1284 |
| | | GUUACdTdT | | | UAAGGGdTdT | |
| D-2862 | S-3121 | CCUUAACUCAUCUG | 1454 | A-3121 | UAUAACAGAUGAG | 1285 |
| | | UUAUCdTdT | | | UUAAGGdTdT | |
| D-2863 | S-3122 | CUUAACUCAUCUGU | 1455 | A-3122 | UGAUAACAGAUGA | 1286 |
| | | UAUCCdTdT | | | GUUAAGdTdT | |
| D-2864 | S-3123 | UUAACUCAUCUGUU | 1456 | A-3123 | UGGAUAACAGAUG | 1287 |
| | | AUCCCdTdT | | | AGUUAAdTdT | |
| D-2865 | S-3124 | UAACUCAUCUGUUA | 1457 | A-3124 | UAGGAUAACAGAU | 1288 |
| | | UCCUCdTdT | | | GAGUUAdTdT | |
| D-2866 | S-3125 | AACUCAUCUGUUAU | 1458 | A-3125 | UCAGGAUAACAGA | 1289 |
| | | CCUGCdTdT | | | UGAGUUdTdT | |
| D-2867 | S-3126 | GUUAUCCUGCUAGC | 1459 | A-3126 | UUACAGCUAGCAG | 1290 |
| | | UGUACdTdT | | | GAUAACdTdT | |
| D-2868 | S-3127 | CUGCUAGCUGUAGA | 1460 | A-3127 | UCAUUUCUACAGC | 1291 |
| | | AAUGCdTdT | | | UAGCAGdTdT | |
| D-2869 | S-3128 | UGCUAGCUGUAGAA | 1461 | A-3128 | UACAUUUCUACAG | 1292 |
| | | AUGUCdTdT | | | CUAGCAdTdT | |
| D-2870 | S-3129 | GCUGUAGAAAUGUA | 1462 | A-3129 | UAGGAUACAUUUC | 1293 |
| | | UCCUCdTdT | | | UACAGCdTdT | |
| D-2871 | S-3130 | CUGUAGAAAUGUAU | 1463 | A-3130 | UCAGGAUACAUUU | 1294 |
| | | CCUGCdTdT | | | CUACAGdTdT | |
| D-2872 | S-3131 | UGUAGAAAUGUAUC | 1464 | A-3131 | UUCAGGAUACAUU | 1295 |
| | | CUGACdTdT | | | UCUACAdTdT | |
| D-2873 | S-3132 | GUAGAAAUGUAUCC | 1465 | A-3132 | UAUCAGGAUACAU | 1296 |
| | | UGAUCdTdT | | | UUCUACdTdT | |
| D-2874 | S-3133 | AAAUGUAUCCUGAU | 1466 | A-3133 | UGUUUAUCAGGAU | 1297 |
| | | AAACCdTdT | | | ACAUUUdTdT | |
| D-2875 | S-3134 | GUAUCCUGAUAAAC | 1467 | A-3134 | UUAAUGUUUAUCA | 1298 |
| | | AUUACdTdT | | | GGAUACdTdT | |
| D-2876 | S-3135 | UUAAACACUGUAAU | 1468 | A-3135 | UUAAGAUUACAGU | 1299 |
| | | CUUACdTdT | | | GUUUAAdTdT | |
| D-2877 | S-3136 | ACUGUAAUCUUAAA | 1469 | A-3136 | UCACUUUUAAGAU | 1300 |
| | | AGUGCdTdT | | | UACAGUdTdT | |
| D-2878 | S-3137 | CUGUAAUCUUAAAA | 1470 | A-3137 | UACACUUUUAAGA | 1301 |
| | | GUGUCdTdT | | | UUACAGdTdT | |
| D-2879 | S-3138 | UGUAAUCUUAAAAG | 1471 | A-3138 | UUACACUUUUAAG | 1302 |
| | | UGUACdTdT | | | AUUACAdTdT | |
| D-2880 | S-3139 | GUAAUCUUAAAAGU | 1472 | A-3139 | UUUACACUUUUAA | 1303 |
| | | GUAACdTdT | | | GAUUACdTdT | |
| D-2881 | S-3140 | CUUAAAAGUGUAAU | 1473 | A-3140 | UCACAAUUACACU | 1304 |
| | | UGUGCdTdT | | | UUUAAGdTdT | |
| D-2882 | S-3141 | UACCUGUAGUGAGA | 1474 | A-3141 | UAGUUUCUCACUA | 1305 |
| | | AACUCdTdT | | | CAGGUAdTdT | |
| D-2883 | S-3142 | UUAUGAUCACUUGG | 1475 | A-3142 | UUCUUCCAAGUGA | 1306 |
| | | AAGACdTdT | | | UCAUAAdTdT | |
| D-2884 | S-3143 | AUGAUCACUUGGAA | 1476 | A-3143 | UAAUCUUCCAAGU | 1307 |
| | | GAUUCdTdT | | | GAUCAUdTdT | |
| D-2885 | S-3144 | AUCACUUGGAAGAU | 1477 | A-3144 | UACAAAUCUUCCA | 1308 |
| | | UUGUCdTdT | | | AGUGAUdTdT | |
| D-2886 | S-3145 | UGGAAGAUUUGUAU | 1478 | A-3145 | UAACUAUACAAAU | 1309 |
| | | AGUUCdTdT | | | CUUCCAdTdT | |
| D-2887 | S-3146 | UAUAAAACUCAGUU | 1479 | A-3146 | UUUUUAACUGAGU | 1310 |
| | | AAAACdTdT | | | UUUAUAdTdT | |
| D-2888 | S-3147 | AAACUCAGUUAAAA | 1480 | A-3147 | UGACAUUUUAACU | 1311 |
| | | UGUCCdTdT | | | GAGUUUdTdT | |
| D-2889 | S-3148 | GUCUGUUUCAAUGA | 1481 | A-3148 | UCAGGUCAUUGAA | 1312 |
| | | CCUGCdTdT | | | ACAGACdTdT | |
| D-2890 | S-3149 | AUGACCUGUAUUUU | 1482 | A-3149 | UUGGCAAAAUACA | 1313 |
| | | GCCACdTdT | | | GGUCAUdTdT | |
| D-2891 | S-3150 | ACCUGUAUUUUGCC | 1483 | A-3150 | UGUCUGGCAAAAU | 1314 |
| | | AGACCdTdT | | | ACAGGUdTdT | |
| D-2892 | S-3151 | CCUGUAUUUUGCCA | 1484 | A-3151 | UAGUCUGGCAAAA | 1315 |
| | | GACUCdTdT | | | UACAGGdTdT | |
| D-2893 | S-3152 | UAAAUCACAGAUGG | 1485 | A-3152 | UAUACCCAUCUGU | 1316 |
| | | GUAUCdTdT | | | GAUUUAdTdT | |
| D-2894 | S-3153 | AUCACAGAUGGGUA | 1486 | A-3153 | UUUAAUACCCAUC | 1317 |
| | | UUAACdTdT | | | UGUGAUdTdT | |
| D-2895 | S-3154 | UCACAGAUGGGUAU | 1487 | A-3154 | UUUUAAUACCCAU | 1318 |
| | | UAAACdTdT | | | CUGUGAdTdT | |
| D-2896 | S-3155 | ACAGAUGGGUAUUA | 1488 | A-3155 | UAGUUUAAUACCC | 1319 |
| | | AACUCdTdT | | | AUCUGUdTdT | |
| D-2897 | S-3156 | CAGAUGGGUAUUAA | 1489 | A-3156 | UAAGUUUAAUACC | 1320 |
| | | ACUUCdTdT | | | CAUCUGdTdT | |
| D-2898 | S-3157 | AGAUGGGUAUUAAA | 1490 | A-3157 | UCAAGUUUAAUAC | 1321 |
| | | CUUGCdTdT | | | CCAUCUdTdT | |
| D-2899 | S-3158 | AUGGGUAUUAAACU | 1491 | A-3158 | UGACAAGUUUAAU | 1322 |
| | | UGUCCdTdT | | | ACCCAUdTdT | |
| D-2900 | S-3159 | UAAACUUGUCAGAA | 1492 | A-3159 | UGAAAUUCUGACA | 1323 |
| | | UUUCCdTdT | | | AGUUUAdTdT | |
| D-2901 | S-3160 | UCAUUCAAGCCUGU | 1493 | A-3160 | UAUUCACAGGCUU | 1324 |
| | | GAAUCdTdT | | | GAAUGAdTdT | |
| D-2902 | S-3161 | CAUUCAAGCCUGUG | 1494 | A-3161 | UUAUUCACAGGCU | 1325 |
| | | AAUACdTdT | | | UGAAUGdTdT | |
| D-2903 | S-3162 | AAUAAAAACCCUGU | 1495 | A-3162 | UCCAUACAGGGUU | 1326 |
| | | AUGGCdTdT | | | UUUAUUdTdT | |
| D-2904 | S-3163 | AUAAAAACCCUGUA | 1496 | A-3163 | UGCCAUACAGGGU | 1327 |
| | | UGGCCdTdT | | | UUUUAUdTdT | |
| D-2905 | S-3164 | AACCCUGUAUGGCA | 1497 | A-3164 | UUAAGUGCCAUAC | 1328 |
| | | CUUACdTdT | | | GAGGUUdTdT | |
| D-2906 | S-3165 | ACCCUGUAUGGCAC | 1498 | A-3165 | UAUAAGUGCCAUA | 1329 |
| | | UUAUCdTdT | | | CAGGGUdTdT | |
| D-2907 | S-3166 | GAGGCUAUUAAAAG | 1499 | A-3166 | UGAUUCUUUUAAU | 1330 |
| | | AAUCCdTdT | | | AGCCUCdTdT | |
| D-2908 | S-3167 | AAAGAAUCCAAAUU | 1500 | A-3167 | UUUUGAAUUUGGA | 1331 |
| | | CAAACdTdT | | | UUCUUUdTdT | |
| D-2909 | S-3168 | GAAUCCAAAUUCAA | 1501 | A-3168 | UUAGUUUGAAUUU | 1332 |
| | | ACUACdTdT | | | GGAUUCdTdT | |

In other embodiments, the siRNA molecules of the present invention targeting SOD1 can be encoded in plasmid vectors, AAV particles, viral genome or other nucleic acid expression vectors for delivery to a cell.

DNA expression plasmids can be used to stably express the siRNA duplexes or dsRNA of the present invention targeting SOD1 in cells and achieve long-term inhibition of the target gene expression. In one aspect, the sense and antisense strands of a siRNA duplex are typically linked by a short spacer sequence leading to the expression of a stem-loop structure termed short hairpin RNA (shRNA). The hairpin is recognized and cleaved by Dicer, thus generating mature siRNA molecules.

According to the present invention, AAV particles comprising the nucleic acids encoding the siRNA molecules targeting SOD1 mRNA are produced, the AAV serotypes may be any of the serotypes listed in Table 1. Non-limiting examples of the AAV serotypes include, AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ8, AAV-DJ, AAV-PHP.A, AAV-PHP.B, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3, AAVG2B4, AAVG2B5 and variants thereof.

In some embodiments, the siRNA duplexes or encoded dsRNA of the present invention suppress (or degrade) SOD1 mRNA. Accordingly, the siRNA duplexes or encoded dsRNA can be used to substantially inhibit SOD1 gene expression in a cell. In some aspects, the inhibition of SOD1 gene expression refers to an inhibition by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

According to the present invention, the siRNA molecules are designed and tested for their ability in reducing SOD1 mRNA levels in cultured cells. Such siRNA molecules may form a duplex such as, but not limited to, include those listed in Table 9. As a non-limiting example, the siRNA duplexes may be siRNA duplex IDs: D-2741 to D-2909.

In one embodiment, the siRNA molecules comprise a miRNA seed match for SOD1 located in the guide strand. In another embodiment, the siRNA molecules comprise a miRNA seed match for SOD1 located in the passenger strand. In yet another embodiment, the siRNA duplexes or encoded dsRNA targeting SOD1 gene do not comprise a seed match for SOD1 located in the guide or passenger strand.

In one embodiment, the siRNA duplexes or encoded dsRNA targeting SOD1 gene may have almost no significant full-length off target effects for the guide strand. In another embodiment, the siRNA duplexes or encoded dsRNA targeting SOD1 gene may have almost no significant full-length off target effects for the passenger strand. The siRNA duplexes or encoded dsRNA targeting SOD1 gene may have less than 1%, 2%, 3%, 4%, 5%, 6%, 7%, 8%, 9%, 10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25% 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off target effects for the passenger strand. In yet another embodiment, the siRNA duplexes or encoded dsRNA targeting SOD1 gene may have almost no significant full-length off target effects for the guide strand or the passenger strand. The siRNA duplexes or encoded dsRNA targeting SOD1 gene may have less than 1%, 2%, 3%, 4%, 5%, 6%, 7%, 8%, 9%, 10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25% 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off target effects for the guide or passenger strand.

In one embodiment, the siRNA duplexes or encoded dsRNA targeting SOD1 gene may have high activity in vitro. In another embodiment, the siRNA molecules may have low activity in vitro. In yet another embodiment, the siRNA duplexes or dsRNA targeting the SOD1 gene may have high guide strand activity and low passenger strand activity in vitro.

In one embodiment, the siRNA molecules targeting SOD1 have a high guide strand activity and low passenger strand activity in vitro. The target knock-down (KD) by the guide strand may be at least 40%, 50%, 60%, 65%, 70%, 75%, 80%, 85%, 90%, 95%, 99%, 99.5% or 100%. The target knock-down by the guide strand may be 40-50%, 45-50%, 50-55%, 50-60%, 60-65%, 60-70%, 60-75%, 60-80%, 60-85%, 60-90%, 60-95%, 60-99%, 60-99.5%, 60-100%, 65-70%, 65-75%, 65-80%, 65-85%, 65-90%, 65-95%, 65-99%, 65-99.5%, 65-100%, 70-75%, 70-80%, 70-85%, 70-90%, 70-95%, 70-99%, 70-99.5%, 70-100%, 75-80%, 75-85%, 75-90%, 75-95%, 75-99%, 75-99.5%, 75-100%, 80-85%, 80-90%, 80-95%, 80-99%, 80-99.5%, 80-100%, 85-90%, 85-95%, 85-99%, 85-99.5%, 85-100%, 90-95%, 90-99%, 90-99.5%, 90-100%, 95-99%, 95-99.5%, 95-100%, 99-99.5%, 99-100% or 99.5-100%. As a non-limiting example, the target knock-down (KD) by the guide strand is greater than 70%. As a non-limiting example, the target knock-down (KD) by the guide strand is greater than 60%.

In one embodiment, the siRNA duplex target SOD1 is designed so there is no miRNA seed match for the sense or antisense sequence to the non-SOD1 sequence.

In one embodiment, the IC₅₀ of the guide strand in the siRNA duplex targeting SOD1 for the nearest off target is greater than 100 multiplied by the IC₅₀ of the guide strand for the on-target gene, SOD1. As a non-limiting example, if the IC₅₀ of the guide strand for the nearest off target is greater than 100 multiplied by the IC₅₀ of the guide strand for the target then the siRNA molecule is said to have high guide strand selectivity for inhibiting SOD1 in vitro.

In one embodiment, the 5′ processing of the guide strand of the siRNA duplex targeting SOD1 has a correct start (n) at the 5′ end at least 75%, 80%, 85%, 90%, 95%, 99% or 100% of the time in vitro or in vivo. As a non-limiting example, the 5′ processing of the guide strand is precise and has a correct start (n) at the 5′ end at least 99% of the time in vitro. As a non-limiting example, the 5′ processing of the guide strand is precise and has a correct start (n) at the 5′ end at least 99% of the time in vivo. As a non-limiting example, the 5′ processing of the guide strand is precise and has a correct start (n) at the 5′ end at least 90% of the time in vitro. As a non-limiting example, the 5′ processing of the guide strand is precise and has a correct start (n) at the 5′ end at least 90% of the time in vivo. As a non-limiting example, the 5′ processing of the guide strand is precise and has a correct start (n) at the 5′ end at least 85% of the time in vitro. As a non-limiting example, the 5′ processing of the guide strand is precise and has a correct start (n) at the 5′ end at least 85% of the time in vivo.

In one embodiment, a passenger-guide strand duplex for SOD1 is considered effective when the pri- or pre-microRNAs demonstrate, by methods known in the art and described herein, greater than 2-fold guide to passenger strand ratio when processing is measured. As a non-limiting examples, the pri- or pre-microRNAs demonstrate great than 2-fold, 3-fold, 4-fold, 5-fold, 6-fold, 7-fold, 8-fold, 9-fold, 10-fold, 11-fold, 12-fold, 13-fold, 14-fold, 15-fold, or 2 to 5-fold, 2 to 10-fold, 2 to 15-fold, 3 to 5-fold, 3 to 10-fold, 3 to 15-fold, 4 to 5-fold, 4 to 10-fold, 4 to 15-fold, 5 to 10-fold, 5 to 15-fold, 6 to 10-fold, 6 to 15-fold, 7 to 10-fold, 7 to 15-fold, 8 to 10-fold, 8 to 15-fold, 9 to 10-fold, 9 to 15-fold, 10 to 15-fold, 11 to 15-fold, 12 to 15-fold, 13 to 15-fold, or 14 to 15-fold guide to passenger strand ratio when processing is measured.

In one embodiment, the siRNA molecules may be used to silence wild type or mutant SOD1 by targeting at least one exon on the SOD1 sequence. The exon may be exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, and/or exon 67.

siRNA Modification

In some embodiments, the siRNA molecules of the present invention, when not delivered as a precursor or DNA, may be chemically modified to modulate some features of RNA molecules, such as, but not limited to, increasing the stability of siRNAs in vivo. The chemically modified siRNA molecules can be used in human therapeutic applications, and are improved without compromising the RNAi activity of the siRNA molecules. As a non-limiting example, the siRNA molecules modified at both the 3′ and the 5′ end of both the sense strand and the antisense strand.

In some aspects, the siRNA duplexes of the present invention may contain one or more modified nucleotides such as, but not limited to, sugar modified nucleotides, nucleobase modifications and/or backbone modifications. In some aspects, the siRNA molecule may contain combined modifications, for example, combined nucleobase and backbone modifications.

In one embodiment, the modified nucleotide may be a sugar-modified nucleotide. Sugar modified nucleotides include, but are not limited to 2′-fluoro, 2′-amino and 2′-thio modified ribonucleotides, e.g. 2′-fluoro modified ribonucleotides. Modified nucleotides may be modified on the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

In one embodiment, the modified nucleotide may be a nucleobase-modified nucleotide.

In one embodiment, the modified nucleotide may be a backbone-modified nucleotide. In some embodiments, the siRNA duplexes of the present invention may further comprise other modifications on the backbone. A normal “backbone”, as used herein, refers to the repeating alternating sugar-phosphate sequences in a DNA or RNA molecule. The deoxyribose/ribose sugars are joined at both the 3′-hydroxyl and 5′-hydroxyl groups to phosphate groups in ester links, also known as “phosphodiester” bonds/linker (PO linkage). The PO backbones may be modified as “phosphorothioate backbone (PS linkage). In some cases, the natural phosphodiester bonds may be replaced by amide bonds but the four atoms between two sugar units are kept. Such amide modifications can facilitate the solid phase synthesis of oligonucleotides and increase the thermodynamic stability of a duplex formed with siRNA complement. See e.g. Mesmaeker et al., Pure & Appl. Chem., 1997, 3, 437-440; the content of which is incorporated herein by reference in its entirety.

Modified bases refer to nucleotide bases such as, for example, adenine, guanine, cytosine, thymine, uracil, xanthine, inosine, and queuosine that have been modified by the replacement or addition of one or more atoms or groups. Some examples of modifications on the nucleobase moieties include, but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguano sine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides.

In one embodiment, the modified nucleotides may be on just the sense strand.

In another embodiment, the modified nucleotides may be on just the antisense strand.

In some embodiments, the modified nucleotides may be in both the sense and antisense strands.

In some embodiments, the chemically modified nucleotide does not affect the ability of the antisense strand to pair with the target mRNA sequence.

In one embodiment, the AAV particle comprising a nucleic acid sequence encoding the siRNA molecules of the present invention may encode siRNA molecules which are polycistronic molecules. The siRNA molecules may additionally comprise one or more linkers between regions of the siRNA molecules.

### Molecular Scaffold

In one embodiment, the siRNA molecules may be encoded in a modulatory polynucleotide which also comprises a molecular scaffold. As used herein a “molecular scaffold” is a framework or starting molecule that forms the sequence or structural basis against which to design or make a subsequent molecule.

In one embodiment, the molecular scaffold comprises at least one 5′ flanking region. As a non-limiting example, the 5′ flanking region may comprise a 5′ flanking sequence which may be of any length and may be derived in whole or in part from wild type microRNA sequence or be a completely artificial sequence.

In one embodiment, the molecular scaffold comprises at least one 3′ flanking region. As a non-limiting example, the 3′ flanking region may comprise a 3′ flanking sequence which may be of any length and may be derived in whole or in part from wild type microRNA sequence or be a completely artificial sequence.

In one embodiment, the molecular scaffold comprises at least one loop motif region. As a non-limiting example, the loop motif region may comprise a sequence which may be of any length.

In one embodiment, the molecular scaffold comprises a 5′ flanking region, a loop motif region and/or a 3′ flanking region.

In one embodiment, at least one siRNA, miRNA or other RNAi agent described herein, may be encoded by a modulatory polynucleotide which may also comprise at least one molecular scaffold. The molecular scaffold may comprise a 5′ flanking sequence which may be of any length and may be derived in whole or in part from wild type microRNA sequence or be completely artificial. The 3′ flanking sequence may mirror the 5′ flanking sequence and/or a 3′ flanking sequence in size and origin. Either flanking sequence may be absent. The 3′ flanking sequence may optionally contain one or more CNNC motifs, where “N” represents any nucleotide.

Forming the stem of a stem loop structure is a minimum of the modulatory polynucleotide encoding at least one siRNA, miRNA or other RNAi agent described herein. In some embodiments, the siRNA, miRNA or other RNAi agent described herein comprises at least one nucleic acid sequence which is in part complementary or will hybridize to a target sequence. In some embodiments the payload is an siRNA molecule or fragment of an siRNA molecule.

In some embodiments, the 5′ arm of the stem loop structure of the modulatory polynucleotide comprises a nucleic acid sequence encoding a sense sequence. Non-limiting examples of sense sequences, or fragments or variants thereof, which may be encoded by the modulatory polynucleotide are described in Table 3 and Table 8.

In some embodiments, the 3′ arm of the stem loop of the modulatory polynucleotide comprises a nucleic acid sequence encoding an antisense sequence. The antisense sequence, in some instances, comprises a “G” nucleotide at the 5′ most end. Non-limiting examples of antisense sequences, or fragments or variants thereof, which may be encoded by the modulatory polynucleotide are described in Table 2 and Table 7.

In other embodiments, the sense sequence may reside on the 3′ arm while the antisense sequence resides on the 5′ arm of the stem of the stem loop structure of the modulatory polynucleotide. Non-limiting examples of sense and antisense sequences which may be encoded by the modulatory polynucleotide are described in Tables 2, 3, 7, and 8.

In one embodiment, the sense and antisense sequences may be completely complementary across a substantial portion of their length. In other embodiments the sense sequence and antisense sequence may be at least 70, 80, 90, 95 or 99% complementarity across independently at least 50, 60, 70, 80, 85, 90, 95, or 99% of the length of the strands.

Neither the identity of the sense sequence nor the homology of the antisense sequence need to be 100% complementarity to the target sequence.

In one embodiment, separating the sense and antisense sequence of the stem loop structure of the modulatory polynucleotide is a loop sequence (also known as a loop motif, linker or linker motif). The loop sequence may be of any length, between 4-30 nucleotides, between 4-20 nucleotides, between 4-15 nucleotides, between 5-15 nucleotides, between 6-12 nucleotides, 6 nucleotides, 7 nucleotides, 8 nucleotides, 9 nucleotides, 10 nucleotides, 11 nucleotides, 12 nucleotides, 13 nucleotides, 14 nucleotides, and/or 15 nucleotides.

In some embodiments, the loop sequence comprises a nucleic acid sequence encoding at least one UGUG motif. In some embodiments, the nucleic acid sequence encoding the UGUG motif is located at the 5′ terminus of the loop sequence.

In one embodiment, spacer regions may be present in the modulatory polynucleotide to separate one or more modules (e.g., 5′ flanking region, loop motif region, 3′ flanking region, sense sequence, antisense sequence) from one another. There may be one or more such spacer regions present.

In one embodiment, a spacer region of between 8-20, i.e., 8, 9, 10, 11, 12, 13, 14, 15, 16, 17, 18, 19, or 20 nucleotides may be present between the sense sequence and a flanking region sequence.

In one embodiment, the length of the spacer region is 13 nucleotides and is located between the 5′ terminus of the sense sequence and the 3′ terminus of the flanking sequence. In one embodiment, a spacer is of sufficient length to form approximately one helical turn of the sequence.

In one embodiment, a spacer region of between 8-20, i.e., 8, 9, 10, 11, 12, 13, 14, 15, 16, 17, 18, 19, or 20 nucleotides may be present between the antisense sequence and a flanking sequence.

In one embodiment, the spacer sequence is between 10-13, i.e., 10, 11, 12 or 13 nucleotides and is located between the 3′ terminus of the antisense sequence and the 5′ terminus of a flanking sequence. In one embodiment, a spacer is of sufficient length to form approximately one helical turn of the sequence.

In one embodiment, the molecular scaffold of the modulatory polynucleotide comprises in the 5′ to 3′ direction, a 5′ flanking sequence, a 5′ arm, a loop motif, a 3′ arm and a 3′ flanking sequence. As a non-limiting example, the 5′ arm may comprise a nucleic acid sequence encoding a sense sequence and the 3′ arm comprises a nucleic acid sequence encoding the antisense sequence. In another non-limiting example, the 5′ arm comprises a nucleic acid sequence encoding the antisense sequence and the 3′ arm comprises a nucleic acid sequence encoding the sense sequence.

In one embodiment, the 5′ arm, sense and/or antisense sequence, loop motif and/or 3′ arm sequence may be altered (e.g., substituting 1 or more nucleotides, adding nucleotides and/or deleting nucleotides). The alteration may cause a beneficial change in the function of the construct (e.g., increase knock-down of the target sequence, reduce degradation of the construct, reduce off target effect, increase efficiency of the payload, and reduce degradation of the payload).

In one embodiment, the molecular scaffold of the modulatory polynucleotides is aligned in order to have the rate of excision of the guide strand (also referred to herein as the antisense strand) be greater than the rate of excision of the passenger strand (also referred to herein as the sense strand). The rate of excision of the guide or passenger strand may be, independently, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99%. As a non-limiting example, the rate of excision of the guide strand is at least 80%. As another non-limiting example, the rate of excision of the guide strand is at least 90%.

In one embodiment, the rate of excision of the guide strand is greater than the rate of excision of the passenger strand. In one aspect, the rate of excision of the guide strand may be at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99% greater than the passenger strand.

In one embodiment, the efficiency of excision of the guide strand is at least 60%, 65%, 70%, 75%, 80%, 85%, 90%, 95%, 99% or more than 99%. As a non-limiting example, the efficiency of the excision of the guide strand is greater than 80%.

In one embodiment, the efficiency of the excision of the guide strand is greater than the excision of the passenger strand from the molecular scaffold. The excision of the guide strand may be 2, 3, 4, 5, 6, 7, 8, 9, 10 or more than 10 times more efficient than the excision of the passenger strand from the molecular scaffold.

In one embodiment, the molecular scaffold comprises a dual-function targeting modulatory polynucleotide. As used herein, a “dual-function targeting” modulatory polynucleotide is a polynucleotide where both the guide and passenger strands knock down the same target or the guide and passenger strands knock down different targets.

In one embodiment, the molecular scaffold of the modulatory polynucleotides described herein may comprise a 5′ flanking region, a loop motif region and a 3′ flanking region. Non-limiting examples of the sequences for the 5′ flanking region, loop motif region (may also be referred to as a linker region) and the 3′ flanking region which may be used, or fragments thereof used, in the modulatory polynucleotides described herein are shown in Tables 10-12.

TABLE 10
5′ Flanking Regions for Molecular Scaffold

| 5′ | | 5′ |
|---|---|---|
| Flanking | | Flanking |
| Region | 5′ Flanking | Region |
| Name | Region Sequence | SEQ ID |
| 5F3 | GTGCTGGGCGGGGGGCGGCGGGCCCT | 1503 |
| | CCCGCAGAACACCATGCGCTCCACGG | |
| | AA | |
| 5F1 | GTGCTGGGCGGGGGGCGGCGGGCCCT | 1504 |
| | CCCGCAGAACACCATGCGCTCTTCGG | |
| | AA | |
| 5F2 | GAAGCAAAGAAGGGGCAGAGGGAGCC | 1505 |
| | CGTGAGCTGAGTGGGCCAGGGACTGG | |
| | GAGAAGGAGTGAGGAGGCAGGGCCGG | |
| | CATGCCTCTGCTGCTGGCCAGA | |
| 5F4 | GGGCCCTCCCGCAGAACACCATGCGC | 1506 |
| | TCCACGGAA | |
| 5F5 | CTCCCGCAGAACACCATGCGCTCCAC | 1507 |
| | GGAA | |
| 5F6 | GTGCTGGGCGGGGGGCGGCGGGCCCT | 1508 |
| | CCCGCAGAACACCATGCGCTCCACGG | |
| | AAG | |
| 5F7 | GTGCTGGGCGGGGGGCGGCGGGCCCT | 1509 |
| | CCCGCAGAACACCATGCGCTCCTCGG | |
| | AA | |
| 5F8 | TTTATGCCTCATCCTCTGAGTGCTGA | 1692 |
| | AGGCTTGCTGTAGGCTGTATGCTG | |
| 5F9 | GTGCTGGGCGGGGGGCGGCGGGCCCT | 1782 |
| | CCCGCAGAACACCATGCGCTCTTCGG | |
| | GA | |

TABLE 11
Loop Motif Regions for Molecular Scaffold

| Loop | | Loop |
|---|---|---|
| Motif | | Motif |
| Region | Loop Motif | Region |
| Name | Region Sequence | SEQ ID |
| L5 | GTGGCCACTGAGAAG | 1510 |
| L1 | TGTGACCTGG | 1511 |
| L2 | TGTGATTTGG | 1512 |
| L3 | GTCTGCACCTGTCACTAG | 1513 |
| L4 | GTGACCCAAG | 1514 |
| L6 | GTGACCCAAT | 1515 |
| L7 | GTGACCCAAC | 1516 |
| L8 | GTGGCCACTGAGAAA | 1517 |
| L9 | TATAATTTGG | 1693 |
| L10 | CCTGACCCAGT | 1694 |

TABLE 12
3′ Flanking Regions for Molecular Scaffold

| 3′ | | 3′ |
|---|---|---|
| Flanking | | Flanking |
| Region | 3′ Flanking | Region |
| Name | Region Sequence | SEQ ID |
| 3F1 | CTGAGGAGCGCCTTGACAGCAGCCAT | 1518 |
| | GGGAGGGCCGCCCCCTACCTCAGTGA | |
| 3F2 | CTGTGGAGCGCCTTGACAGCAGCCAT | 1519 |
| | GGGAGGGCCGCCCCCTACCTCAGTGA | |
| 3F3 | TGGCCGTGTAGTGCTACCCAGCGCTG | 1520 |
| | GCTGCCTCCTCAGCATTGCAATTCCT | |
| | CTCCCATCTGGGCACCAGTCAGCTAC | |
| | CCTGGTGGGAATCTGGGTAGCC | |
| 3F4 | CTGAGGAGCGCCTTGACAGCAGCCAT | 1521 |
| | GGGAGGGCC | |
| 3F5 | CTGCGGAGCGCCTTGACAGCAGCCAT | 1522 |
| | GGGAGGGCCGCCCCCTACCTCAGTGA | |
| 3F6 | AGTGTATGATGCCTGTTACTAGCATT | 1695 |
| | CACATGGAACAAATTGCTGCCGTG | |
| 3F7 | TCCTGAGGAGCGCCTTGACAGCAGCC | 1783 |
| | ATGGGAGGGCCGCCCCCTACCTCAGT | |
| | GA | |

In one embodiment, the molecular scaffold may comprise at least one 5′ flanking region, fragment or variant thereof listed in Table 10. As a non-limiting example, the 5′ flanking region may be 5F1, 5F2, 5F3, 5F4, 5F5, 5F6, 5F7, 5F8, or 5F9.

In one embodiment, the molecular scaffold may comprise at least one 5F1 flanking region.

In one embodiment, the molecular scaffold may comprise at least one 5F2 flanking region.

In one embodiment, the molecular scaffold may comprise at least one 5F3 flanking region.

In one embodiment, the molecular scaffold may comprise at least one 5F4 flanking region.

In one embodiment, the molecular scaffold may comprise at least one 5F5 flanking region.

In one embodiment, the molecular scaffold may comprise at least one 5F6 flanking region.

In one embodiment, the molecular scaffold may comprise at least one 5F7 flanking region.

In one embodiment, the molecular scaffold may comprise at least one 5F8 flanking region.

In one embodiment, the molecular scaffold may comprise at least one 5F9 flanking region.

In one embodiment, the molecular scaffold may comprise at least one loop motif region, fragment or variant thereof listed in Table 11. As a non-limiting example, the loop motif region may be L1, L2, L3, L4, L5, L6, L7, L8, L9, or L10.

In one embodiment, the molecular scaffold may comprise at least one L1 loop motif region.

In one embodiment, the molecular scaffold may comprise at least one L2 loop motif region.

In one embodiment, the molecular scaffold may comprise at least one L3 loop motif region.

In one embodiment, the molecular scaffold may comprise at least one L4 loop motif region.

In one embodiment, the molecular scaffold may comprise at least one L5 loop motif region.

In one embodiment, the molecular scaffold may comprise at least one L6 loop motif region.

In one embodiment, the molecular scaffold may comprise at least one L7 loop motif region.

In one embodiment, the molecular scaffold may comprise at least one L8 loop motif region.

In one embodiment, the molecular scaffold may comprise at least one L9 loop motif region.

In one embodiment, the molecular scaffold may comprise at least one L10 loop motif region.

In one embodiment, the molecular scaffold may comprise at least one 3′ flanking region, fragment or variant thereof listed in Table 12. As a non-limiting example, the 3′ flanking region may be 3F1, 3F2, 3F3, 3F4, 3F5, 3F6, or 3F7.

In one embodiment, the molecular scaffold may comprise at least one 3F1 flanking region.

In one embodiment, the molecular scaffold may comprise at least one 3F2 flanking region.

In one embodiment, the molecular scaffold may comprise at least one 3F3 flanking region.

In one embodiment, the molecular scaffold may comprise at least one 3F4 flanking region.

In one embodiment, the molecular scaffold may comprise at least one 3F5 flanking region.

In one embodiment, the molecular scaffold may comprise at least one 3F6 flanking region.

In one embodiment, the molecular scaffold may comprise at least one 3F7 flanking region.

In one embodiment, the molecular scaffold may comprise at least one 5′ flanking region, fragment or variant thereof, and at least one loop motif region, fragment or variant thereof, as described in Tables 10 and 11. As a non-limiting example, the 5′ flanking region and the loop motif region may be 5F1 and L1, 5F1 and L2, 5F1 and L3, 5F1 and L4, 5F1 and L5, 5F1 and L6, 5F1 and L7, 5F1 and L8, 5F1 and L9, 5F1 and L10, 5F2 and L1, 5F2 and L2, 5F2 and L3, 5F2 and L4, 5F2 and L5, 5F2 and L6, 5F2 and L7, 5F2 and L8, 5F2 and L9, 5F2 and L10, 5F3 and L1, 5F3 and L2, 5F3 and L3, 5F3 and L4, 5F3 and L5, 5F3 and L6, 5F3 and L7, 5F3 and L8, 5F3 and L9, 5F3 and L10, 5F4 and L1, 5F4 and L2, 5F4 and L3, 5F4 and L4, 5F4 and L5, 5F4 and L6, 5F4 and L7, 5F4 and L8, 5F4 and L9, 5F4 and L10, 5F5 and L1, 5F5 and L2, 5F5 and L3, 5F5 and L4, 5F5 and L5, 5F5 and L6, 5F5 and L7, 5F5 and L8, 5F5 and L9, 5F5 and L10, 5F6 and L1, 5F6 and L2, 5F6 and L3, 5F6 and L4, 5F6 and L5, 5F6 and L6, 5F6 and L7, 5F6 and L8, 5F6 and L9, 5F6 and L10, 5F7 and L1, 5F7 and L2, 5F7 and L3, 5F7 and L4, 5F7 and L5, 5F7 and L6, 5F7 and L7, 5F7 and L8, 5F7 and L9, 5F7 and L10, 5F8 and L1, 5F8 and L2, 5F8 and L3, 5F8 and L4, 5F8 and L5, 5F8 and L6, 5F8 and L7, 5F8 and L8, 5F8 and L9, 5F8 and L10, 5F9 and L1, 5F9 and L2, 5F9 and L3, 5F9 and L4, 5F9 and L5, 5F9 and L6, 5F9 and L7, 5F9 and L8, 5F9 and L9, and 5F9 and L10.

In one embodiment, the molecular scaffold may comprise at least one 5F2 flanking region and at least one L1 loop motif region.

In one embodiment, the molecular scaffold may comprise at least one 5F1 flanking region and at least one L4 loop motif region.

In one embodiment, the molecular scaffold may comprise at least one 5F7 flanking region and at least one L8 loop motif region.

In one embodiment, the molecular scaffold may comprise at least one 5F3 flanking region and at least one L4 loop motif region.

In one embodiment, the molecular scaffold may comprise at least one 5F3 flanking region and at least one L5 loop motif region.

In one embodiment, the molecular scaffold may comprise at least one 5F4 flanking region and at least one L4 loop motif region.

In one embodiment, the molecular scaffold may comprise at least one 5F3 flanking region and at least one L7 loop motif region.

In one embodiment, the molecular scaffold may comprise at least one 5F5 flanking region and at least one L4 loop motif region.

In one embodiment, the molecular scaffold may comprise at least one 5F6 flanking region and at least one L4 loop motif region.

In one embodiment, the molecular scaffold may comprise at least one 5F3 flanking region and at least one L6 loop motif region.

In one embodiment, the molecular scaffold may comprise at least one 5F7 flanking region and at least one L4 loop motif region.

In one embodiment, the molecular scaffold may comprise at least one 5F2 flanking region and at least one L2 loop motif region.

In one embodiment, the molecular scaffold may comprise at least one 5F1 flanking region and at least one L1 loop motif region.

In one embodiment, the molecular scaffold may comprise at least one 5F1 flanking region and at least one L2 loop motif region.

In one embodiment, the molecular scaffold may comprise at least one 3′ flanking region, fragment or variant thereof, and at least one motif region, fragment or variant thereof, as described in Tables 11 and 12. As a non-limiting example, the 3′ flanking region and the loop motif region may be 3F1 and L1, 3F1 and L2, 3F1 and L3, 3F1 and L4, 3F1 and L5, 3F1 and L6, 3F1 and L7, 3F1 and L8, 3F1 and L9, 3F1 and L10, 3F2 and L1, 3F2 and L2, 3F2 and L3, 3F2 and L4, 3F2 and L5, 3F2 and L6, 3F2 and L7, 3F2 and L8, 3F2 and L9, 3F2 and L10, 3F3 and L1, 3F3 and L2, 3F3 and L3, 3F3 and L4, 3F3 and L5, 3F3 and L6, 3F3 and L7, 3F3 and L8, 3F3 and L9, 3F3 and L10, 3F4 and L1, 3F4 and L2, 3F4 and L3, 3F4 and L4, 3F4 and L5, 3F4 and L6, 3F4 and L7, 3F4 and L8, 3F4 and L9, 3F4 and L10, 3F5 and L1, 3F5 and L2, 3F5 and L3, 3F5 and L4, 3F5 and L5, 3F5 and L6, 3F5 and L7, 3F5 and L8, 3F5 and L9, 3F5 and L10, 3F6 and L1, 3F6 and L2, 3F6 and L3, 3F6 and L4, 3F6 and L5, 3F6 and L6, 3F6 and L7, 3F6 and L8, 3F6 and L9, 3F6 and L10, 3F7 and L1, 3F7 and L2, 3F7 and L3, 3F7 and L4, 3F7 and L5, 3F7 and L6, 3F7 and L7, 3F7 and L8, 3F7 and L9, and 3F7 and L10.

In one embodiment, the molecular scaffold may comprise at least one L1 loop motif region and at least one 3F2 flanking region.

In one embodiment, the molecular scaffold may comprise at least one L4 loop motif region and at least one 3F1 flanking region.

In one embodiment, the molecular scaffold may comprise at least one L8 loop motif region and at least one 3F5 flanking region.

In one embodiment, the molecular scaffold may comprise at least one L5 loop motif region and at least 3F1 flanking region.

In one embodiment, the molecular scaffold may comprise at least one L4 loop motif region and at least one 3F4 flanking region.

In one embodiment, the molecular scaffold may comprise at least one L7 loop motif region and at least one 3F1 flanking region.

In one embodiment, the molecular scaffold may comprise at least one L6 loop motif region and at least one 3F1 flanking region.

In one embodiment, the molecular scaffold may comprise at least one L4 loop motif region and at least one 3F5 flanking region.

In one embodiment, the molecular scaffold may comprise at least one L2 loop motif region and at least one 3F2 flanking region.

In one embodiment, the molecular scaffold may comprise at least one L1 loop motif region and at least one 3F3 flanking region.

In one embodiment, the molecular scaffold may comprise at least one L5 loop motif region and at least one 3F4 flanking region.

In one embodiment, the molecular scaffold may comprise at least one L1 loop motif region and at least one 3F1 flanking region.

In one embodiment, the molecular scaffold may comprise at least one L2 loop motif region and at least one 3F1 flanking region.

In one embodiment, the molecular scaffold may comprise at least one 5′ flanking region, fragment or variant thereof, and at least one 3′ flanking region, fragment or variant thereof, as described in Tables 10 and 12. As a non-limiting example, the flanking regions may be 5F1 and 3F1, 5F1 and 3F2, 5F1 and 3F3, 5F1 and 3F4, 5F1 and 3F5, 5F1 and 3F6, 5F1 and 3F7, 5F2 and 3F1, 5F2 and 3F2, 5F2 and 3F3, 5F2 and 3F4, 5F2 and 3F5, 5F2 and 3F6, 5F2 and 3F7, 5F3 and 3F1, 5F3 and 3F2, 5F3 and 3F3, 5F3 and 3F4, 5F3 and 3F5, 5F3 and 3F6, 5F3 and 3F7, 5F4 and 3F1, 5F4 and 3F2, 5F4 and 3F3, 5F4 and 3F4, 5F4 and 3F5, 5F4 and 3F6, 5F4 and 3F7, 5F5 and 3F1, 5F5 and 3F2, 5F5 and 3F3, 5F5 and 3F4, 5F5 and 3F5, 5F5 and 3F6, 5F5 and 3F7, 5F6 and 3F1, 5F6 and 3F2, 5F6 and 3F3, 5F6 and 3F4, 5F6 and 3F5, 5F6 and 3F6, 5F6 and 3F7, 5F7 and 3F1, 5F7 and 3F2, 5F7 and 3F3, 5F7 and 3F4, 5F7 and 3F5, 5F7 and 3F6, 5F7 and 3F7, 5F8 and 3F1, 5F8 and 3F2, 5F8 and 3F3, 5F8 and 3F4, 5F8 and 3F5, 5F8 and 3F6, and 5F8 and 3F7. 5F9 and 3F1, 5F9 and 3F2, 5F9 and 3F3, 5F9 and 3F4, 5F9 and 3F5, 5F9 and 3F6, and 5F9 and 3F7

In one embodiment, the molecular scaffold may comprise at least one 5F2 5′ flanking region and at least one 3F2 3′ flanking region.

In one embodiment, the molecular scaffold may comprise at least one 5F1 5′ flanking region and at least one 3F1 3′ flanking region.

In one embodiment, the molecular scaffold may comprise at least one 5F7 5′ flanking region and at least one 3F5 3′ flanking region.

In one embodiment, the molecular scaffold may comprise at least one 5F3 5′ flanking region and at least one 3F1 3′ flanking region.

In one embodiment, the molecular scaffold may comprise at least one 5F4 5′ flanking region and at least one 3F4 3′ flanking region.

In one embodiment, the molecular scaffold may comprise at least one 5F5 5′ flanking region and at least one 3F4 3′ flanking region.

In one embodiment, the molecular scaffold may comprise at least one 5F6 5′ flanking region and at least one 3F1 3′ flanking region.

In one embodiment, the molecular scaffold may comprise at least one 5F2 5′ flanking region and at least one 3F3 3′ flanking region.

In one embodiment, the molecular scaffold may comprise at least one 5F3 5′ flanking region and at least one 3F4 3′ flanking region.

In one embodiment, the molecular scaffold may comprise at least one 5F1 5′ flanking region and at least one 3F2 3′ flanking region.

In one embodiment, the molecular scaffold may comprise at least one 5′ flanking region, fragment or variant thereof, at least one loop motif region, fragment or variant thereof, and at least one 3′ flanking region as described in Tables 10-12. As a non-limiting example, the flanking and loop motif regions may be 5F1, L1 and 3F1; 5F1, L1 and 3F2; 5F1, L1 and 3F3; 5F1, L1 and 3F4; 5F1, L1 and 3F5; 5F1, L1 and 3F6; 5F1, L1 and 3F7; 5F2, L1 and 3F1; 5F2, L1 and 3F2; 5F2, L1 and 3F3; 5F2, L1 and 3F4; 5F2, L1 and 3F5; 5F2, L1 and 3F6; 5F2, L1 and 3F7; 5F3, L1 and 3F1; 5F3, L1 and 3F2; 5F3, L1 and 3F3; 5F3, L1 and 3F4; 5F3, L1 and 3F5; 5F3, L1 and 3F6; 5F3, L1 and 3F7; 5F4, L1 and 3F1; 5F4, L1 and 3F2; 5F4, L1 and 3F3; 5F4, L1 and 3F4; 5F4, L1 and 3F5; 5F4, L1 and 3F6; 5F4, L1 and 3F7; 5F5, L1 and 3F1; 5F5, L1 and 3F2; 5F5, L1 and 3F3; 5F5, L1 and 3F4; 5F5, L1 and 3F5; 5F5, L1 and 3F6; 5F5, L1 and 3F7; 5F6, L1 and 3F1; 5F6, L1 and 3F2; 5F6, L1 and 3F3; 5F6, L1 and 3F4; 5F6, L1 and 3F5; 5F6, L1 and 3F6; 5F6, L1 and 3F7; 5F7, L1 and 3F1; 5F7, L1 and 3F2; 5F7, L1 and 3F3; 5F7, L1 and 3F4; 5F7, L1 and 3F5; 5F7, L1 and 3F6; 5F7, L1 and 3F7; 5F8, L1 and 3F1; 5F8, L1 and 3F2; 5F8, L1 and 3F3; 5F8, L1 and 3F4; 5F8, L1 and 3F5; 5F8, L1 and 3F6; 5F8, L1 and 3F7; 5F9, L1 and 3F1; 5F9, L1 and 3F2; 5F9, L1 and 3F3; 5F9, L1 and 3F4; 5F9, L1 and 3F5; 5F9, L1 and 3F6; 5F9, L1 and 3F7; 5F1, L2 and 3F1; 5F1, L2 and 3F2; 5F1, L2 and 3F3; 5F1, L2 and 3F4; 5F1, L2 and 3F5; 5F1, L2 and 3F6; 5F1, L2 and 3F7; 5F2, L2 and 3F1; 5F2, L2 and 3F2; 5F2, L2 and 3F3; 5F2, L2 and 3F4; 5F2, L2 and 3F5; 5F2, L2 and 3F6; 5F2, L2 and 3F7; 5F3, L2 and 3F1; 5F3, L2 and 3F2; 5F3, L2 and 3F3; 5F3, L2 and 3F4; 5F3, L2 and 3F5; 5F3, L2 and 3F6; 5F3, L2 and 3F7; 5F4, L2 and 3F1; 5F4, L2 and 3F2; 5F4, L2 and 3F3; 5F4, L2 and 3F4; 5F4, L2 and 3F5; 5F4, L2 and 3F6; 5F4, L2 and 3F7; 5F5, L2 and 3F1; 5F5, L2 and 3F2; 5F5, L2 and 3F3; 5F5, L2 and 3F4; 5F5, L2 and 3F5; 5F5, L2 and 3F6; 5F5, L2 and 3F7; 5F6, L2 and 3F1; 5F6, L2 and 3F2; 5F6, L2 and 3F3; 5F6, L2 and 3F4; 5F6, L2 and 3F5; 5F6, L2 and 3F6; 5F6, L2 and 3F7; 5F7, L2 and 3F1; 5F7, L2 and 3F2; 5F7, L2 and 3F3; 5F7, L2 and 3F4; 5F7, L2 and 3F5; 5F7, L2 and 3F6; 5F7, L2 and 3F7; 5F8, L2 and 3F1; 5F8, L2 and 3F2; 5F8, L2 and 3F3; 5F8, L2 and 3F4; 5F8, L2 and 3F5; 5F8, L2 and 3F6; 5F8, L2 and 3F7; 5F9, L2 and 3F1; 5F9, L2 and 3F2; 5F9, L2 and 3F3; 5F9, L2 and 3F4; 5F9, L2 and 3F5; 5F9, L2 and 3F6; 5F9, L2 and 3F7; 5F1, L3 and 3F1; 5F1, L3 and 3F2; 5F1, L3 and 3F3; 5F1, L3 and 3F4; 5F1, L3 and 3F5; 5F1, L3 and 3F6; 5F1, L3 and 3F7; 5F2, L3 and 3F1; 5F2, L3 and 3F2; 5F2, L3 and 3F3; 5F2, L3 and 3F4; 5F2, L3 and 3F5; 5F2, L3 and 3F6; 5F2, L3 and 3F7; 5F3, L3 and 3F1; 5F3, L3 and 3F2; 5F3, L3 and 3F3; 5F3, L3 and 3F4; 5F3, L3 and 3F5; 5F3, L3 and 3F6; 5F3, L3 and 3F7; 5F4, L3 and 3F1; 5F4, L3 and 3F2; 5F4, L3 and 3F3; 5F4, L3 and 3F4; 5F4, L3 and 3F5; 5F4, L3 and 3F6; 5F4, L3 and 3F7; 5F5, L3 and 3F1; 5F5, L3 and 3F2; 5F5, L3 and 3F3; 5F5, L3 and 3F4; 5F5, L3 and 3F5; 5F5, L3 and 3F6; 5F5, L3 and 3F7; 5F6, L3 and 3F1; 5F6, L3 and 3F2; 5F6, L3 and 3F3; 5F6, L3 and 3F4; 5F6, L3 and 3F5; 5F6, L3 and 3F6; 5F6, L3 and 3F7; 5F7, L3 and 3F1; 5F7, L3 and 3F2; 5F7, L3 and 3F3; 5F7, L3 and 3F4; 5F7, L3 and 3F5; 5F7, L3 and 3F6; 5F7, L3 and 3F7; 5F8, L3 and 3F1; 5F8, L3 and 3F2; 5F8, L3 and 3F3; 5F8, L3 and 3F4; 5F8, L3 and 3F5; 5F8, L3 and 3F6; 5F8, L3 and 3F7; 5F9, L3 and 3F1; 5F9, L3 and 3F2; 5F9, L3 and 3F3; 5F9, L3 and 3F4; 5F9, L3 and 3F5; 5F9, L3 and 3F6; 5F9, L3 and 3F7; 5F1, L4 and 3F1; 5F1, L4 and 3F2; 5F1, L4 and 3F3; 5F1, L4 and 3F4; 5F1, L4 and 3F5; 5F1, L4 and 3F6; 5F1, L4 and 3F7; 5F2, L4 and 3F1; 5F2, L4 and 3F2; 5F2, L4 and 3F3; 5F2, L4 and 3F4; 5F2, L4 and 3F5; 5F2, L4 and 3F6; 5F2, L4 and 3F7; 5F3, L4 and 3F1; 5F3, L4 and 3F2; 5F3, L4 and 3F3; 5F3, L4 and 3F4; 5F3, L4 and 3F5; 5F3, L4 and 3F6; 5F3, L4 and 3F7; 5F4, L4 and 3F1; 5F4, L4 and 3F2; 5F4, L4 and 3F3; 5F4, L4 and 3F4; 5F4, L4 and 3F5; 5F4, L4 and 3F6; 5F4, L4 and 3F7; 5F5, L4 and 3F1; 5F5, L4 and 3F2; 5F5, L4 and 3F3; 5F5, L4 and 3F4; 5F5, L4 and 3F5; 5F5, L4 and 3F6; 5F5, L4 and 3F7; 5F6, L4 and 3F1; 5F6, L4 and 3F2; 5F6, L4 and 3F3; 5F6, L4 and 3F4; 5F6, L4 and 3F5; 5F6, L4 and 3F6; 5F6, L4 and 3F7; 5F7, L4 and 3F1; 5F7, L4 and 3F2; 5F7, L4 and 3F3; 5F7, L4 and 3F4; 5F7, L4 and 3F5; 5F7, L4 and 3F6; 5F7, L4 and 3F7; 5F8, L4 and 3F1; 5F8, L4 and 3F2; 5F8, L4 and 3F3; 5F8, L4 and 3F4; 5F8, L4 and 3F5; 5F8, L4 and 3F6; 5F8, L4 and 3F7; 5F9, L4 and 3F1; 5F9, L4 and 3F2; 5F9, L4 and 3F3; 5F9, L4 and 3F4; 5F9, L4 and 3F5; 5F9, L4 and 3F6; 5F9, L4 and 3F7; 5F1, L5 and 3F1; 5F1, L5 and 3F2; 5F1, L5 and 3F3; 5F1, L5 and 3F4; 5F1, L5 and 3F5; 5F1, L5 and 3F6; 5F1, L5 and 3F7; 5F2, L5 and 3F1; 5F2, L5 and 3F2; 5F2, L5 and 3F3; 5F2, L5 and 3F4; 5F2, L5 and 3F5; 5F2, L5 and 3F6; 5F2, L5 and 3F7; 5F3, L5 and 3F1; 5F3, L5 and 3F2; 5F3, L5 and 3F3; 5F3, L5 and 3F4; 5F3, L5 and 3F5; 5F3, L5 and 3F6; 5F3, L5 and 3F7; 5F4, L5 and 3F1; 5F4, L5 and 3F2; 5F4, L5 and 3F3; 5F4, L5 and 3F4; 5F4, L5 and 3F5; 5F4, L5 and 3F6; 5F4, L5 and 3F7; 5F5, L5 and 3F1; 5F5, L5 and 3F2; 5F5, L5 and 3F3; 5F5, L5 and 3F4; 5F5, L5 and 3F5; 5F5, L5 and 3F6; 5F5, L5 and 3F7; 5F6, L5 and 3F1; 5F6, L5 and 3F2; 5F6, L5 and 3F3; 5F6, L5 and 3F4; 5F6, L5 and 3F5; 5F6, L5 and 3F6; 5F6, L5 and 3F7; 5F7, L5 and 3F1; 5F7, L5 and 3F2; 5F7, L5 and 3F3; 5F7, L5 and 3F4; 5F7, L5 and 3F5; 5F7, L5 and 3F6; 5F7, L5 and 3F7; 5F8, L5 and 3F1; 5F8, L5 and 3F2; 5F8, L5 and 3F3; 5F8, L5 and 3F4; 5F8, L5 and 3F5; 5F8, L5 and 3F6; 5F8, L5 and 3F7; 5F9, L5 and 3F1; 5F9, L5 and 3F2; 5F9, L5 and 3F3; 5F9, L5 and 3F4; 5F9, L5 and 3F5; 5F9, L5 and 3F6; 5F9, L5 and 3F7; 5F1, L6 and 3F1; 5F1, L6 and 3F2; 5F1, L6 and 3F3; 5F1, L6 and 3F4; 5F1, L6 and 3F5; 5F1, L6 and 3F6; 5F1, L6 and 3F7; 5F2, L6 and 3F1; 5F2, L6 and 3F2; 5F2, L6 and 3F3; 5F2, L6 and 3F4; 5F2, L6 and 3F5; 5F2, L6 and 3F6; 5F2, L6 and 3F7; 5F3, L6 and 3F1; 5F3, L6 and 3F2; 5F3, L6 and 3F3; 5F3, L6 and 3F4; 5F3, L6 and 3F5; 5F3, L6 and 3F6; 5F3, L6 and 3F7; 5F4, L6 and 3F1; 5F4, L6 and 3F2; 5F4, L6 and 3F3; 5F4, L6 and 3F4; 5F4, L6 and 3F5; 5F4, L6 and 3F6; 5F4, L6 and 3F7; 5F5, L6 and 3F1; 5F5, L6 and 3F2; 5F5, L6 and 3F3; 5F5, L6 and 3F4; 5F5, L6 and 3F5; 5F5, L6 and 3F6; 5F5, L6 and 3F7; 5F6, L6 and 3F1; 5F6, L6 and 3F2; 5F6, L6 and 3F3; 5F6, L6 and 3F4; 5F6, L6 and 3F5; 5F6, L6 and 3F6; 5F6, L6 and 3F7; 5F7, L6 and 3F1; 5F7, L6 and 3F2; 5F7, L6 and 3F3; 5F7, L6 and 3F4; 5F7, L6 and 3F5; 5F7, L6 and 3F6; 5F7, L6 and 3F7; 5F8, L6 and 3F1; 5F8, L6 and 3F2; 5F8, L6 and 3F3; 5F8, L6 and 3F4; 5F8, L6 and 3F5; 5F8, L6 and 3F6; 5F8, L6 and 3F7; 5F9, L6 and 3F1; 5F9, L6 and 3F2; 5F9, L6 and 3F3; 5F9, L6 and 3F4; 5F9, L6 and 3F5; 5F9, L6 and 3F6; 5F9, L6 and 3F7; 5F1, L7 and 3F1; 5F1, L7 and 3F2; 5F1, L7 and 3F3; 5F1, L7 and 3F4; 5F1, L7 and 3F5; 5F1, L7 and 3F6; 5F1, L7 and 3F7; 5F2, L7 and 3F1; 5F2, L7 and 3F2; 5F2, L7 and 3F3; 5F2, L7 and 3F4; 5F2, L7 and 3F5; 5F2, L7 and 3F6; 5F2, L7 and 3F7; 5F3, L7 and 3F1; 5F3, L7 and 3F2; 5F3, L7 and 3F3; 5F3, L7 and 3F4; 5F3, L7 and 3F5; 5F3, L7 and 3F6; 5F3, L7 and 3F7; 5F4, L7 and 3F1; 5F4, L7 and 3F2; 5F4, L7 and 3F3; 5F4, L7 and 3F4; 5F4, L7 and 3F5; 5F4, L7 and 3F6; 5F4, L7 and 3F7; 5F5, L7 and 3F1; 5F5, L7 and 3F2; 5F5, L7 and 3F3; 5F5, L7 and 3F4; 5F5, L7 and 3F5; 5F5, L7 and 3F6; 5F5, L7 and 3F7; 5F6, L7 and 3F1; 5F6, L7 and 3F2; 5F6, L7 and 3F3; 5F6, L7 and 3F4; 5F6, L7 and 3F5; 5F6, L7 and 3F6; 5F6, L7 and 3F7; 5F7, L7 and 3F1; 5F7, L7 and 3F2; 5F7, L7 and 3F3; 5F7, L7 and 3F4; 5F7, L7 and 3F5; 5F7, L7 and 3F6; 5F7, L7 and 3F7; 5F8, L7 and 3F1; 5F8, L7 and 3F2; 5F8, L7 and 3F3; 5F8, L7 and 3F4; 5F8, L7 and 3F5; 5F8, L7 and 3F6; 5F8, L7 and 3F7; 5F9, L7 and 3F1; 5F9, L7 and 3F2; 5F9, L7 and 3F3; 5F9, L7 and 3F4; 5F9, L7 and 3F5; 5F9, L7 and 3F6; 5F9, L7 and 3F7; 5F1, L8 and 3F1; 5F1, L8 and 3F2; 5F1, L8 and 3F3; 5F1, L8 and 3F4; 5F1, L8 and 3F5; 5F1, L8 and 3F6; 5F1, L8 and 3F7; 5F2, L8 and 3F1; 5F2, L8 and 3F2; 5F2, L8 and 3F3; 5F2, L8 and 3F4; 5F2, L8 and 3F5; 5F2, L8 and 3F6; 5F2, L8 and 3F7; 5F3, L8 and 3F1; 5F3, L8 and 3F2; 5F3, L8 and 3F3; 5F3, L8 and 3F4; 5F3, L8 and 3F5; 5F3, L8 and 3F6; 5F3, L8 and 3F7; 5F4, L8 and 3F1; 5F4, L8 and 3F2; 5F4, L8 and 3F3; 5F4, L8 and 3F4; 5F4, L8 and 3F5; 5F4, L8 and 3F6; 5F4, L8 and 3F7; 5F5, L8 and 3F1; 5F5, L8 and 3F2; 5F5, L8 and 3F3; 5F5, L8 and 3F4; 5F5, L8 and 3F5; 5F5, L8 and 3F6; 5F5, L8 and 3F7; 5F6, L8 and 3F1; 5F6, L8 and 3F2; 5F6, L8 and 3F3; 5F6, L8 and 3F4; 5F6, L8 and 3F5; 5F6, L8 and 3F6; 5F6, L8 and 3F7; 5F7, L8 and 3F1; 5F7, L8 and 3F2; 5F7, L8 and 3F3; 5F7, L8 and 3F4; 5F7, L8 and 3F5; 5F7, L8 and 3F6; 5F7, L8 and 3F7; 5F8, L8 and 3F1; 5F8, L8 and 3F2; 5F8, L8 and 3F3; 5F8, L8 and 3F4; 5F8, L8 and 3F5; 5F8, L8 and 3F6; 5F8, L8 and 3F7; 5F9, L8 and 3F1; 5F9, L8 and 3F2; 5F9, L8 and 3F3; 5F9, L8 and 3F4; 5F9, L8 and 3F5; 5F9, L8 and 3F6; 5F9, L8 and 3F7; 5F1, L9 and 3F1; 5F1, L9 and 3F2; 5F1, L9 and 3F3; 5F1, L9 and 3F4; 5F1, L9 and 3F5; 5F1, L9 and 3F6; 5F1, L9 and 3F7; 5F2, L9 and 3F1; 5F2, L9 and 3F2; 5F2, L9 and 3F3; 5F2, L9 and 3F4; 5F2, L9 and 3F5; 5F2, L9 and 3F6; 5F2, L9 and 3F7; 5F3, L9 and 3F1; 5F3, L9 and 3F2; 5F3, L9 and 3F3; 5F3, L9 and 3F4; 5F3, L9 and 3F5; 5F3, L9 and 3F6; 5F3, L9 and 3F7; 5F4, L9 and 3F1; 5F4, L9 and 3F2; 5F4, L9 and 3F3; 5F4, L9 and 3F4; 5F4, L9 and 3F5; 5F4, L9 and 3F6; 5F4, L9 and 3F7; 5F5, L9 and 3F1; 5F5, L9 and 3F2; 5F5, L9 and 3F3; 5F5, L9 and 3F4; 5F5, L9 and 3F5; 5F5, L9 and 3F6; 5F5, L9 and 3F7; 5F6, L9 and 3F1; 5F6, L9 and 3F2; 5F6, L9 and 3F3; 5F6, L9 and 3F4; 5F6, L9 and 3F5; 5F6, L9 and 3F6; 5F6, L9 and 3F7; 5F7, L9 and 3F1; 5F7, L9 and 3F2; 5F7, L9 and 3F3; 5F7, L9 and 3F4; 5F7, L9 and 3F5; 5F7, L9 and 3F6; 5F7, L9 and 3F7; 5F8, L9 and 3F1; 5F8, L9 and 3F2; 5F8, L9 and 3F3; 5F8, L9 and 3F4; 5F8, L9 and 3F5; 5F8, L9 and 3F6; 5F8, L9 and 3F7; 5F9, L9 and 3F1; 5F9, L9 and 3F2; 5F9, L9 and 3F3; 5F9, L9 and 3F4; 5F9, L9 and 3F5; 5F9, L9 and 3F6; 5F9, L9 and 3F7; 5F1, L10 and 3F1; 5F1, L10 and 3F2; 5F1, L10 and 3F3; 5F1, L10 and 3F4; 5F1, L10 and 3F5; 5F1, L10 and 3F6; 5F1, L10 and 3F7; 5F2, L10 and 3F1; 5F2, L10 and 3F2; 5F2, L10 and 3F3; 5F2, L10 and 3F4; 5F2, L10 and 3F5; 5F2, L10 and 3F6; 5F2, L10 and 3F7; 5F3, L10 and 3F1; 5F3, L10 and 3F2; 5F3, L10 and 3F3; 5F3, L10 and 3F4; 5F3, L10 and 3F5; 5F3, L10 and 3F6; 5F3, L10 and 3F7; 5F4, L10 and 3F1; 5F4, L10 and 3F2; 5F4, L10 and 3F3; 5F4, L10 and 3F4; 5F4, L10 and 3F5; 5F4, L10 and 3F6; 5F4, L10 and 3F7; 5F5, L10 and 3F1; 5F5, L10 and 3F2; 5F5, L10 and 3F3; 5F5, L10 and 3F4; 5F5, L10 and 3F5; 5F5, L10 and 3F6; 5F5, L10 and 3F7; 5F6, L10 and 3F1; 5F6, L10 and 3F2; 5F6, L10 and 3F3; 5F6, L10 and 3F4; 5F6, L10 and 3F5; 5F6, L10 and 3F6; 5F6, L10 and 3F7; 5F7, L10 and 3F1; 5F7, L10 and 3F2; 5F7, L10 and 3F3; 5F7, L10 and 3F4; 5F7, L10 and 3F5; 5F7, L10 and 3F6; 5F7, L10 and 3F7; 5F8, L10 and 3F1; 5F8, L10 and 3F2; 5F8, L10 and 3F3; 5F8, L10 and 3F4; 5F8, L10 and 3F5; 5F8, L10 and 3F6; 5F8, L10 and 3F7; 5F9, L10 and 3F1; 5F9, L10 and 3F2; 5F9, L10 and 3F3; 5F9, L10 and 3F4; 5F9, L10 and 3F5; 5F9, L10 and 3F6; and 5F9, L10 and 3F7.

In one embodiment, the molecular scaffold may comprise at least one 5F2 5′ flanking region, at least one L1 loop motif region, and at least one 3F2 3′ flanking region.

In one embodiment, the molecular scaffold may comprise at least one 5F1 5′ flanking region, at least one L4 loop motif region, and at least one 3F1 3′ flanking region.

In one embodiment, the molecular scaffold may comprise at least one 5F7 5′ flanking region, at least one L8 loop motif region, and at least one 3F5 3′ flanking region.

In one embodiment, the molecular scaffold may comprise at least one 5F3 5′ flanking region, at least one L4 loop motif region, and at least one 3F1 3′ flanking region.

In one embodiment, the molecular scaffold may comprise at least one 5F3 5′ flanking region, at least one L5 loop motif region, and at least one 3F1 3′ flanking region.

In one embodiment, the molecular scaffold may comprise at least one 5F4 5′ flanking region, at least one L4 loop motif region, and at least one 3F4 3′ flanking region.

In one embodiment, the molecular scaffold may comprise at least one 5F3 5′ flanking region, at least one L7 loop motif region, and at least one 3F1 3′ flanking region.

In one embodiment, the molecular scaffold may comprise at least one 5F5 5′ flanking region, at least one L4 loop motif region, and at least one 3F4 3′ flanking region.

In one embodiment, the molecular scaffold may comprise at least one 5F6 5′ flanking region, at least one L4 loop motif region, and at least one 3F1 3′ flanking region.

In one embodiment, the molecular scaffold may comprise at least one 5F3 5′ flanking region, at least one L6 loop motif region, and at least one 3F1 3′ flanking region.

In one embodiment, the molecular scaffold may comprise at least one 5F7 5′ flanking region, at least one L4 loop motif region, and at least one 3F5 3′ flanking region.

In one embodiment, the molecular scaffold may comprise at least one 5F2 5′ flanking region, at least one L2 loop motif region, and at least one 3F2 3′ flanking region.

In one embodiment, the molecular scaffold may comprise at least one 5F2 5′ flanking region, at least one L1 loop motif region, and at least one 3F3 3′ flanking region.

In one embodiment, the molecular scaffold may comprise at least one 5F3 5′ flanking region, at least one L5 loop motif region, and at least one 3F4 3′ flanking region.

In one embodiment, the molecular scaffold may comprise at least one 5F1 5′ flanking region, at least one L1 loop motif region, and at least one 3F1 3′ flanking region.

In one embodiment, the molecular scaffold may comprise at least one 5F1 5′ flanking region, at least one L2 loop motif region, and at least one 3F1 3′ flanking region.

In one embodiment, the molecular scaffold may comprise at least one 5F1 5′ flanking region, at least one L1 loop motif region, and at least one 3F2 3′ flanking region.

In one embodiment, the molecular scaffold may comprise at least one 5F2 5′ flanking region, at least one L3 loop motif region, and at least one 3F3 3′ flanking region.

In one embodiment, the molecular scaffold may be a natural pri-miRNA scaffold. As a non-limiting example, the molecular scaffold may be a scaffold derived from the human miR155 scaffold.

In one embodiment, the molecular scaffold may comprise one or more linkers known in the art. The linkers may separate regions or one molecular scaffold from another. As a non-limiting example, the molecular scaffold may be polycistronic.

Modulatory Polynucleotide Comprising Molecular Scaffold and siRNA Molecules Targeting HTT

In one embodiment, the modulatory polynucleotide may comprise 5′ and 3′ flanking regions, loop motif region, and nucleic acid sequences encoding sense sequence and antisense sequence as described in Tables 13 and 14. In Tables 13 and 14, the DNA sequence identifier for the passenger and guide strands are described as well as the 5′ and 3′ Flanking Regions and the Loop region (also referred to as the linker region). In Tables 13 and 14, the “miR” component of the name of the sequence does not necessarily correspond to the sequence numbering of miRNA genes (e.g., VOYHTmiR-102 is the name of the sequence and does not necessarily mean that miR-102 is part of the sequence).

TABLE 13
HTT Modulatory Polynucleotide Sequence Regions (5′ to 3′)

| Modulatory | 5′ Flanking to | 5′ | | | | 3′ |
|---|---|---|---|---|---|---|
| Polynucleotide | 3′ Flanking | Flanking | Passenger | Loop | uide | Flanking |
| Construct Name | SEQ ID NO | SEQ ID NO | SEQ ID NO | SEQ ID NO | SEQ ID NO | SEQ ID NO |
| VOYHTmiR-102.214 | 1523 | 1504 | 1636 | 1511 | 677 | 1518 |
| VOYHTmiR-104.214 | 1524 | 1504 | 1643 | 1511 | 677 | 1518 |
| VOYHTmiR-109.214 | 1525 | 1504 | 1650 | 1512 | 677 | 1518 |
| VOYHTmiR-114.214 | 1526 | 1504 | 1657 | 1511 | 677 | 1519 |
| VOYHTmiR-116.214 | 1527 | 1504 | 1650 | 1511 | 677 | 1519 |
| VOYHTmiR-127.214 | 1528 | 1505 | 1650 | 1513 | 674 | 1520 |
| VOYHTmiR-102.218 | 1529 | 1504 | 1637 | 1511 | 678 | 1518 |
| VOYHTmiR-104.218 | 1530 | 1504 | 1644 | 1511 | 678 | 1518 |
| VOYHTmiR-109.218 | 1531 | 1504 | 1651 | 1512 | 678 | 1518 |
| VOYHTmiR-114.218 | 1532 | 1504 | 1658 | 1511 | 678 | 1519 |
| VOYHTmiR-116.218 | 1533 | 1504 | 1651 | 1511 | 678 | 1519 |
| VOYHTmiR-127.218 | 1534 | 1505 | 1651 | 1513 | 678 | 1520 |
| VOYHTmiR-102.219.o | 1535 | 1504 | 1620 | 1511 | 673 | 1518 |
| VOYHTmiR-104.219.o | 1536 | 1504 | 1623 | 1511 | 673 | 1518 |
| VOYHTmiR-109.219.o | 1537 | 1504 | 1620 | 1512 | 673 | 1518 |
| VOYHTmiR-114.219 | 1538 | 1504 | 1626 | 1511 | 673 | 1519 |
| VOYHTmiR-116.219.o | 1539 | 1504 | 1629 | 1511 | 673 | 1519 |
| VOYHTmiR-127.219.o | 1540 | 1505 | 1620 | 1513 | 673 | 1520 |
| VOYHTmiR-102.219.n | 1541 | 1504 | 1632 | 1511 | 673 | 1518 |
| VOYHTmiR-104.219.n | 1542 | 1504 | 1633 | 1511 | 673 | 1518 |
| VOYHTmiR-109.219.n | 1543 | 1504 | 1632 | 1512 | 673 | 1518 |
| VOYHTmiR-116.219.n | 1544 | 1504 | 1634 | 1511 | 673 | 1519 |
| VOYHTmiR-127.219.n | 1545 | 1505 | 1632 | 1513 | 673 | 1520 |
| VOYHTmiR-102.257 | 1546 | 1504 | 1638 | 1511 | 679 | 1518 |
| VOYHTmiR-104.257 | 1547 | 1504 | 1645 | 1511 | 679 | 1518 |
| VOYHTmiR-109.257 | 1548 | 1504 | 1652 | 1512 | 679 | 1518 |
| VOYHTmiR-114.257 | 1549 | 1504 | 1659 | 1511 | 679 | 1519 |
| VOYHTmiR-116.257 | 1550 | 1504 | 1652 | 1511 | 679 | 1519 |
| VOYHTmiR-127.257 | 1551 | 1505 | 1652 | 1513 | 679 | 1520 |
| VOYHTmiR-102.894 | 1552 | 1504 | 1621 | 1511 | 674 | 1518 |
| VOYHTmiR-104.894 | 1553 | 1504 | 1624 | 1511 | 674 | 1518 |
| VOYHTmiR-109.894 | 1554 | 1504 | 1621 | 1512 | 674 | 1518 |
| VOYHTmiR-114.894 | 1555 | 1504 | 1627 | 1511 | 674 | 1519 |
| VOYHTmiR-116.894 | 1556 | 1504 | 1630 | 1511 | 674 | 1519 |
| VOYHTmiR-127.894 | 1557 | 1505 | 1621 | 1513 | 674 | 1520 |
| VOYHTmiR-102.907 | 1558 | 1504 | 1641 | 1511 | 682 | 1518 |
| VOYHTmiR-104.907 | 1559 | 1504 | 1648 | 1511 | 682 | 1518 |
| VOYHTmiR-109.907 | 1560 | 1504 | 1655 | 1512 | 682 | 1518 |
| VOYHTmiR-114.907 | 1561 | 1504 | 1662 | 1511 | 682 | 1519 |
| VOYHTmiR-116.907 | 1562 | 1504 | 1655 | 1511 | 682 | 1519 |
| VOYHTmiR-127.907 | 1563 | 1505 | 1655 | 1513 | 682 | 1520 |
| VOYHTmiR-102.372 | 1564 | 1504 | 1639 | 1511 | 680 | 1518 |
| VOYHTmiR-104.372 | 1565 | 1504 | 1646 | 1511 | 680 | 1518 |
| VOYHTmiR-109.372 | 1566 | 1504 | 1653 | 1512 | 680 | 1518 |
| VOYHTmiR-114.372 | 1567 | 1504 | 1660 | 1511 | 680 | 1519 |
| VOYHTmiR-116.372 | 1568 | 1504 | 1653 | 1511 | 680 | 1519 |
| VOYHTmiR-127.372 | 1569 | 1505 | 1653 | 1513 | 680 | 1520 |
| VOYHTmiR-102.425 | 1570 | 1504 | 1640 | 1511 | 681 | 1518 |
| VOYHTmiR-104.425 | 1571 | 1504 | 1647 | 1511 | 681 | 1518 |
| VOYHTmiR-109.425 | 1572 | 1504 | 1654 | 1512 | 681 | 1518 |
| VOYHTmiR-114.425 | 1573 | 1504 | 1661 | 1511 | 681 | 1519 |
| VOYHTmiR-116.425 | 1574 | 1504 | 1654 | 1511 | 681 | 1519 |
| VOYHTmiR-127.425 | 1575 | 1505 | 1654 | 1513 | 681 | 1520 |
| VOYHTmiR-102.032 | 1576 | 1504 | 1664 | 1511 | 684 | 1518 |
| VOYHTmiR-104.032 | 1577 | 1504 | 1666 | 1511 | 684 | 1518 |
| VOYHTmiR-109.032 | 1578 | 1504 | 1668 | 1512 | 684 | 1518 |
| VOYHTmiR-114.032 | 1579 | 1504 | 1670 | 1511 | 684 | 1519 |
| VOYHTmiR-116.032 | 1580 | 1504 | 1668 | 1511 | 684 | 1519 |
| VOYHTmiR-127.032 | 1581 | 1505 | 1668 | 1513 | 684 | 1520 |
| VOYHTmiR-102.020 | 1582 | 1504 | 1663 | 1511 | 683 | 1518 |
| VOYHTmiR-104.020 | 1583 | 1504 | 1665 | 1511 | 683 | 1518 |
| VOYHTmiR-109.020 | 1584 | 1504 | 1667 | 1512 | 683 | 1518 |
| VOYHTmiR-114.020 | 1585 | 1504 | 1669 | 1511 | 683 | 1519 |
| VOYHTmiR-116.020 | 1586 | 1504 | 1667 | 1511 | 683 | 1519 |
| VOYHTmiR-127.020 | 1587 | 1505 | 1667 | 1513 | 683 | 1520 |
| VOYHTmiR-102.016 | 1588 | 1504 | 1635 | 1511 | 676 | 1518 |
| VOYHTmiR-104.016 | 1589 | 1504 | 1642 | 1511 | 676 | 1518 |
| VOYHTmiR-109.016 | 1590 | 1504 | 1649 | 1512 | 676 | 1518 |
| VOYHTmiR-114.016 | 1591 | 1504 | 1656 | 1511 | 676 | 1519 |
| VOYHTmiR-116.016 | 1592 | 1504 | 1649 | 1511 | 676 | 1519 |
| VOYHTmiR-127.016 | 1593 | 1505 | 1649 | 1513 | 676 | 1520 |
| VOYHTmiR-102.579 | 1594 | 1504 | 1622 | 1511 | 675 | 1518 |
| VOYHTmiR-104.579 | 1595 | 1504 | 1625 | 1511 | 675 | 1518 |
| VOYHTmiR-109.579 | 1596 | 1504 | 1622 | 1512 | 675 | 1518 |
| VOYHTmiR-114.579 | 1597 | 1504 | 1628 | 1511 | 675 | 1519 |
| VOYHTmiR-116.579 | 1598 | 1504 | 1631 | 1511 | 675 | 1519 |
| VOYHTmiR-127.579 | 1599 | 1505 | 1622 | 1513 | 675 | 1520 |
| VOYHTmiR-104.579.1 | 1600 | 1504 | 1671 | 1514 | 675 | 1518 |
| VOYHTmiR-104.579.2 | 1601 | 1503 | 1671 | 1514 | 675 | 1518 |
| VOYHTmiR-104.579.3 | 1602 | 1503 | 1671 | 1510 | 675 | 1518 |
| VOYHTmiR-104.579.4 | 1603 | 1506 | 1671 | 1514 | 675 | 1521 |
| VOYHTmiR-104.579.6 | 1604 | 1507 | 1671 | 1514 | 675 | 1521 |
| VOYHTmiR-104.579.7 | 1605 | 1508 | 1671 | 1514 | 685 | 1518 |
| VOYHTmiR-104.579.8 | 1606 | 1503 | 1672 | 1515 | 675 | 1518 |
| VOYHTmiR-104.579.9 | 1607 | 1509 | 1671 | 1514 | 675 | 1522 |
| VOYHTmiR-102.020 | 1608 | 1504 | 1663 | 1511 | 683 | 1518 |
| VOYHTmiR-102.032 | 1609 | 1504 | 1664 | 1511 | 684 | 1518 |
| VOYHTmiR-104.020 | 1610 | 1504 | 1665 | 1511 | 683 | 1518 |
| VOYHTmiR-104.032 | 1611 | 1504 | 1666 | 1511 | 684 | 1518 |
| VOYHTmiR-109.020 | 1612 | 1504 | 1667 | 1512 | 683 | 1518 |
| VOYHTmiR-109.032 | 1613 | 1504 | 1668 | 1512 | 684 | 1518 |
| VOYHTmiR-114.020 | 1614 | 1504 | 1669 | 1511 | 683 | 1519 |
| VOYHTmiR-114.032 | 1615 | 1504 | 1670 | 1511 | 684 | 1519 |
| VOYHTmiR-116.020 | 1616 | 1504 | 1667 | 1511 | 683 | 1519 |
| VOYHTmiR-116.032 | 1617 | 1504 | 1668 | 1511 | 684 | 1519 |
| VOYHTmiR-127.020 | 1618 | 1505 | 1667 | 1513 | 683 | 1520 |
| VOYHTmiR-127.032 | 1619 | 1505 | 1668 | 1513 | 684 | 1520 |

TABLE 14
HTT Modulatory Polynucleotide Sequence Region (5′ to 3′)

| | 5′ Flanking to | 5′ | | | | 3′ |
|---|---|---|---|---|---|---|
| | 3′ Flanking | Flanking | Passenger | Loop | Guide | Flanking |
| Name | SEQ ID NO | SEQ ID NO | SEQ ID NO | SEQ ID NO | SEQ ID NO | SEQ ID NO |
| VOYHTmiR-104.579.5 | 1686 | 1503 | 1688 | 1516 | 1690 | 1518 |
| VOYHTmiR-104.579.10 | 1687 | 1509 | 1689 | 1517 | 1691 | 1532 |

Modulatory Polynucleotide Comprising Molecular Scaffold and siRNA Molecules Targeting SOD1

In one embodiment, the modulatory polynucleotide may comprise 5′ and 3′ flanking regions, loop motif region, and nucleic acid sequences encoding sense sequence and antisense sequence as described in Tables 15 and 16. In Tables 15 and 16, the DNA sequence identifier for the passenger and guide strands are described as well as the 5′ and 3′ Flanking Regions and the Loop region (also referred to as the linker region). In Tables 15 and 16, the “miR” component of the name of the sequence does not necessarily correspond to the sequence numbering of miRNA genes (e.g., VOYSOD1miR-102 is the name of the sequence and does not necessarily mean that miR-102 is part of the sequence).

TABLE 15
SOD1 Modulatory Polynucleotide Sequence Regions (5′ to 3′)

| Modulatory | 5′ Flanking to | 5′ | | | | 3′ |
|---|---|---|---|---|---|---|
| Polynucleotide | 3′ Flanking | Flanking | Passenger | Loop | uide | Flanking |
| Construct Name | SEQ ID NO | SEQ ID NO | SEQ ID NO | SEQ ID NO | SEQ ID NO | SEQ ID NO |
| VOYSOD1miR-101 | 1696 | 1692 | 1746 | 1510 | 747 | 1695 |
| VOYSOD1miR-102 | 1697 | 1503 | 1746 | 1510 | 747 | 1518 |
| VOYSOD1miR-103 | 1698 | 1503 | 1748 | 1510 | 747 | 1518 |
| VOYSOD1miR-104 | 1699 | 1503 | 1749 | 1510 | 747 | 1518 |
| VOYSOD1miR-105 | 1700 | 1503 | 1750 | 1510 | 747 | 1518 |
| VOYSOD1miR-106 | 1701 | 1503 | 1751 | 1510 | 747 | 1518 |
| VOYSOD1miR-107 | 1702 | 1503 | 1752 | 1510 | 747 | 1518 |
| VOYSOD1miR-108 | 1703 | 1503 | 1754 | 1510 | 747 | 1518 |
| VOYSOD1miR-109 | 1704 | 1503 | 1746 | 1511 | 747 | 1518 |
| VOYSOD1miR-110 | 1705 | 1503 | 1746 | 1693 | 747 | 1518 |
| VOYSOD1miR-111 | 1706 | 1503 | 1753 | 1694 | 747 | 1518 |
| VOYSOD1miR-112 | 1707 | 1503 | 1746 | 1510 | 747 | 1519 |
| VOYSOD1miR-113 | 1708 | 1503 | 1748 | 1510 | 747 | 1519 |
| VOYSOD1miR-114 | 1709 | 1503 | 1751 | 1510 | 747 | 1519 |
| VOYSOD1miR-115 | 1710 | 1503 | 1753 | 1694 | 747 | 1519 |
| VOYSOD1miR-116 | 1711 | 1503 | 1749 | 1510 | 747 | 1519 |
| VOYSOD1miR-117 | 1712 | 1503 | 1755 | 1510 | 756 | 1518 |
| VOYSOD1miR-118 | 1713 | 1503 | 1757 | 1510 | 758 | 1518 |
| VOYSOD1miR-119 | 1714 | 1503 | 1759 | 1510 | 760 | 1518 |
| VOYSOD1miR-127 | 1715 | 1504 | 1746 | 1512 | 747 | 1520 |
| VOYSOD1miR-102.860 | 1716 | 1503 | 1761 | 1510 | 762 | 1518 |
| VOYSOD1miR-102.861 | 1717 | 1503 | 1763 | 1510 | 764 | 1518 |
| VOYSOD1miR-102.866 | 1718 | 1503 | 1765 | 1510 | 760 | 1518 |
| VOYSOD1miR-102.870 | 1719 | 1503 | 1766 | 1510 | 767 | 1518 |
| VOYSOD1miR-102.823 | 1720 | 1503 | 1768 | 1510 | 758 | 1518 |
| VOYSOD1miR-104.860 | 1721 | 1503 | 1769 | 1510 | 762 | 1518 |
| VOYSOD1miR-104.861 | 1722 | 1503 | 1770 | 1510 | 764 | 1518 |
| VOYSOD1miR-104.866 | 1723 | 1503 | 1771 | 1510 | 760 | 1518 |
| VOYSOD1miR-104.870 | 1724 | 1503 | 1772 | 1510 | 767 | 1518 |
| VOYSOD1miR-104.823 | 1725 | 1503 | 1773 | 1510 | 758 | 1518 |
| VOYSOD1miR-109.860 | 1726 | 1503 | 1761 | 1511 | 762 | 1518 |
| VOYSOD1miR-104.861 | 1727 | 1503 | 1763 | 1511 | 764 | 1518 |
| VOYSOD1miR-104.866 | 1728 | 1503 | 1765 | 1511 | 760 | 1518 |
| VOYSOD1miR-109.870 | 1729 | 1503 | 1766 | 1511 | 767 | 1518 |
| VOYSOD1miR-109.823 | 1730 | 1503 | 1768 | 1511 | 758 | 1518 |
| VOYSOD1miR-114.860 | 1731 | 1503 | 1774 | 1510 | 762 | 1519 |
| VOYSOD1miR-114.861 | 1732 | 1503 | 1775 | 1510 | 764 | 1519 |
| VOYSOD1miR-114.866 | 1733 | 1503 | 1776 | 1510 | 760 | 1519 |
| VOYSOD1miR-114.870 | 1734 | 1503 | 1777 | 1510 | 767 | 1519 |
| VOYSOD1miR-114.823 | 1735 | 1503 | 1778 | 1510 | 758 | 1519 |
| VOYSOD1miR-116.860 | 1736 | 1503 | 1769 | 1510 | 762 | 1519 |
| VOYSOD1miR-116.861 | 1737 | 1503 | 1770 | 1510 | 764 | 1519 |
| VOYSOD1miR-116.866 | 1738 | 1503 | 1779 | 1510 | 760 | 1519 |
| VOYSOD1miR-116.870 | 1739 | 1503 | 1772 | 1510 | 767 | 1519 |
| VOYSOD1miR-116.823 | 1740 | 1503 | 1773 | 1510 | 758 | 1519 |
| VOYSOD1miR-127.860 | 1741 | 1504 | 1780 | 1512 | 762 | 1520 |
| VOYSOD1miR-127.861 | 1742 | 1504 | 1763 | 1512 | 764 | 1520 |
| VOYSOD1miR-127.866 | 1743 | 1504 | 1765 | 1512 | 760 | 1520 |
| VOYSOD1miR-127.870 | 1744 | 1504 | 1766 | 1512 | 767 | 1520 |
| VOYSOD1miR-127.823 | 1745 | 1504 | 1781 | 1512 | 758 | 1520 |

TABLE 16
SOD1 Modulatory Polynucleotide Sequence Region (5′ to 3′)

| | 5′ Flanking to | 5′ | | | | 3′ |
|---|---|---|---|---|---|---|
| | 3′ Flanking | Flanking | Passenger | Loop | Guide | Flanking |
| Name | SEQ ID NO | SEQ ID NO | SEQ ID NO | SEQ ID NO | SEQ ID NO | SEQ ID NO |
| VOYSOD1miR-120 | 1784 | 1782 | 1785 | 1511 | 1786 | 1783 |

### AAV Particles Comprising Modulatory Polynucleotides

In one embodiment, the AAV particle comprises a viral genome with a payload region comprising a modulatory polynucleotide sequences. In such an embodiment, a viral genome encoding more than one polypeptide may be replicated and packaged into a viral particle. A target cell transduced with a viral particle comprising a modulatory polynucleotide may express the encoded sense and/or antisense sequences in a single cell.

In some embodiments, the AAV particles are useful in the field of medicine for the treatment, prophylaxis, palliation or amelioration of neurological diseases and/or disorders.

In one embodiment, the AAV particles comprising modulatory polynucleotide sequence which comprises a nucleic acid sequence encoding at least one siRNA molecule may be introduced into mammalian cells.

Where the AAV particle payload region comprises a modulatory polynucleotide, the modulatory polynucleotide may comprise sense and/or antisense sequences to knock down a target gene. The AAV viral genomes encoding modulatory polynucleotides described herein may be useful in the fields of human disease, viruses, infections veterinary applications and a variety of in vivo and in vitro settings.

In one embodiment, the AAV particle viral genome may comprise at least one inverted terminal repeat (ITR) region. The ITR region(s) may, independently, have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR region for the viral genome may be 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140, 120-125, 120-130, 120-145, 125-130, 125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises an ITR that is about 105 nucleotides in length. As a non-limiting example, the viral genome comprises an ITR that is about 141 nucleotides in length. As a non-limiting example, the viral genome comprises an ITR that is about 130 nucleotides in length.

In one embodiment, the AAV particle viral genome may comprises two inverted terminal repeat (ITR) regions. Each of the ITR regions may independently have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR regions for the viral genome may be 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140, 120-125, 120-130, 120-145, 125-130, 125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises an ITR that is about 105 nucleotides in length and 141 nucleotides in length. As a non-limiting example, the viral genome comprises an ITR that is about 105 nucleotides in length and 130 nucleotides in length. As a non-limiting example, the viral genome comprises an ITR that is about 130 nucleotides in length and 141 nucleotides in length.

In one embodiment, the AAV particle viral genome may comprise at least one sequence region as described in Tables 17-24. The regions may be located before or after any of the other sequence regions described herein.

In one embodiment, the AAV particle viral genome comprises at least one inverted terminal repeat (ITR) sequence region. Non-limiting examples of ITR sequence regions are described in Table 17.

TABLE 17
Inverted Terminal Repeat (ITR) Sequence Regions

| Sequence Region Name | SEQ ID NO |
|---|---|
| ITR1 | 1787 |
| ITR2 | 1788 |
| ITR3 | 1789 |
| ITR4 | 1790 |

In one embodiment, the AAV particle viral genome comprises two ITR sequence regions. In one embodiment, the ITR sequence regions are the ITR1 sequence region and the ITR3 sequence region. In one embodiment, the ITR sequence regions are the ITR1 sequence region and the ITR4 sequence region. In one embodiment, the ITR sequence regions are the ITR2 sequence region and the ITR3 sequence region. In one embodiment, the ITR sequence regions are the ITR2 sequence region and the ITR4 sequence region.

In one embodiment, the AAV particle viral genome may comprise at least one multiple cloning site (MCS) sequence region. The MCS region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the MCS region for the viral genome may be 2-10, 5-10, 5-15, 10-20, 10-30, 10-40, 15-20, 15-25, 20-30, 20-40, 20-50, 25-30, 25-35, 30-40, 30-50, 30-60, 35-40, 35-45, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides. As a non-limiting example, the viral genome comprises a MCS region that is about 5 nucleotides in length. As a non-limiting example, the viral genome comprises a MCS region that is about 10 nucleotides in length. As a non-limiting example, the viral genome comprises a MCS region that is about 14 nucleotides in length. As a non-limiting example, the viral genome comprises a MCS region that is about 18 nucleotides in length. As a non-limiting example, the viral genome comprises a MCS region that is about 73 nucleotides in length. As a non-limiting example, the viral genome comprises a MCS region that is about 121 nucleotides in length.

In one embodiment, the AAV particle viral genome comprises at least one multiple cloning site (MCS) sequence regions. Non-limiting examples of MCS sequence regions are described in Table 18.

TABLE 18
Multiple Cloning Site (MCS) Sequence Regions

| Sequence Region Name | SEQ ID NO or Sequence |
|---|---|
| MCS1 | 1791 |
| MCS2 | 1792 |
| MCS3 | 1793 |
| MCS4 | 1794 |
| MCS5 | TCGAG |
| MCS6 | 1795 |

In one embodiment, the AAV particle viral genome comprises one MCS sequence region. In one embodiment, the MCS sequence region is the MCS1 sequence region. In one embodiment, the MCS sequence region is the MCS2 sequence region. In one embodiment, the MCS sequence region is the MCS3 sequence region. In one embodiment, the MCS sequence region is the MCS4 sequence region. In one embodiment, the MCS sequence region is the MCS5 sequence region. In one embodiment, the MCS sequence region is the MCS6 sequence region.

In one embodiment, the AAV particle viral genome comprises two MCS sequence regions. In one embodiment, the two MCS sequence regions are the MCS1 sequence region and the MCS2 sequence region. In one embodiment, the two MCS sequence regions are the MCS1 sequence region and the MCS3 sequence region. In one embodiment, the two MCS sequence regions are the MCS1 sequence region and the MCS4 sequence region. In one embodiment, the two MCS sequence regions are the MCS1 sequence region and the MCS5 sequence region. In one embodiment, the two MCS sequence regions are the MCS1 sequence region and the MCS6 sequence region. In one embodiment, the two MCS sequence regions are the MCS2 sequence region and the MCS3 sequence region. In one embodiment, the two MCS sequence regions are the MCS2 sequence region and the MCS4 sequence region. In one embodiment, the two MCS sequence regions are the MCS2 sequence region and the MCS5 sequence region. In one embodiment, the two MCS sequence regions are the MCS2 sequence region and the MCS6 sequence region. In one embodiment, the two MCS sequence regions are the MCS3 sequence region and the MCS4 sequence region. In one embodiment, the two MCS sequence regions are the MCS3 sequence region and the MCS5 sequence region. In one embodiment, the two MCS sequence regions are the MCS3 sequence region and the MCS6 sequence region. In one embodiment, the two MCS sequence regions are the MCS4 sequence region and the MCS5 sequence region. In one embodiment, the two MCS sequence regions are the MCS4 sequence region and the MCS6 sequence region. In one embodiment, the two MCS sequence regions are the MCS5 sequence region and the MCS6 sequence region.

In one embodiment, the AAV particle viral genome comprises two or more MCS sequence regions.

In one embodiment, the AAV particle viral genome comprises three MCS sequence regions. In one embodiment, the three MCS sequence regions are the MCS1 sequence region, the MCS2 sequence region, and the MCS3 sequence region. In one embodiment, the three MCS sequence regions are the MCS1 sequence region, the MCS2 sequence region, and the MCS4 sequence region. In one embodiment, the three MCS sequence regions are the MCS1 sequence region, the MCS2 sequence region, and the MCS5 sequence region. In one embodiment, the three MCS sequence regions are the MCS1 sequence region, the MCS2 sequence region, and the MCS6 sequence region. In one embodiment, the three MCS sequence regions are the MCS1 sequence region, the MCS3 sequence region, and the MCS4 sequence region. In one embodiment, the three MCS sequence regions are the MCS1 sequence region, the MCS3 sequence region, and the MCS5 sequence region. In one embodiment, the three MCS sequence regions are the MCS1 sequence region, the MCS3 sequence region, and the MCS6 sequence region. In one embodiment, the three MCS sequence regions are the MCS1 sequence region, the MCS4 sequence region, and the MCS5 sequence region. In one embodiment, the three MCS sequence regions are the MCS1 sequence region, the MCS4 sequence region, and the MCS6 sequence region. In one embodiment, the three MCS sequence regions are the MCS1 sequence region, the MCS5 sequence region, and the MCS6 sequence region. In one embodiment, the three MCS sequence regions are the MCS2 sequence region, the MCS3 sequence region, and the MCS4 sequence region. In one embodiment, the three MCS sequence regions are the MCS2 sequence region, the MCS3 sequence region, and the MCS5 sequence region. In one embodiment, the three MCS sequence regions are the MCS2 sequence region, the MCS3 sequence region, and the MCS6 sequence region. In one embodiment, the three MCS sequence regions are the MCS2 sequence region, the MCS4 sequence region, and the MCS5 sequence region. In one embodiment, the three MCS sequence regions are the MCS2 sequence region, the MCS4 sequence region, and the MCS6 sequence region. In one embodiment, the three MCS sequence regions are the MCS2 sequence region, the MCS5 sequence region, and the MCS6 sequence region. In one embodiment, the three MCS sequence regions are the MCS3 sequence region, the MCS4 sequence region, and the MCS5 sequence region. In one embodiment, the three MCS sequence regions are the MCS3 sequence region, the MCS4 sequence region, and the MCS6 sequence region. In one embodiment, the three MCS sequence regions are the MCS3 sequence region, the MCS5 sequence region, and the MCS6 sequence region. In one embodiment, the three MCS sequence regions are the MCS4 sequence region, the MCS5 sequence region, and the MCS6 sequence region.

In one embodiment, the AAV particle viral genome may comprise at least one multiple filler sequence region. The filler region(s) may, independently, have a length such as, but not limited to, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, and 3250 nucleotides. The length of any filler region for the viral genome may be 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, and 3200-3250 nucleotides. As a non-limiting example, the viral genome comprises a filler region that is about 55 nucleotides in length. As a non-limiting example, the viral genome comprises a filler region that is about 56 nucleotides in length. As a non-limiting example, the viral genome comprises a filler region that is about 97 nucleotides in length. As a non-limiting example, the viral genome comprises a filler region that is about 103 nucleotides in length. As a non-limiting example, the viral genome comprises a filler region that is about 105 nucleotides in length. As a non-limiting example, the viral genome comprises a filler region that is about 357 nucleotides in length. As a non-limiting example, the viral genome comprises a filler region that is about 363 nucleotides in length. As a non-limiting example, the viral genome comprises a filler region that is about 712 nucleotides in length. As a non-limiting example, the viral genome comprises a filler region that is about 714 nucleotides in length. As a non-limiting example, the viral genome comprises a filler region that is about 1203 nucleotides in length. As a non-limiting example, the viral genome comprises a filler region that is about 1209 nucleotides in length. As a non-limiting example, the viral genome comprises a filler region that is about 1512 nucleotides in length. As a non-limiting example, the viral genome comprises a filler region that is about 1519 nucleotides in length. As a non-limiting example, the viral genome comprises a filler region that is about 2395 nucleotides in length. As a non-limiting example, the viral genome comprises a filler region that is about 2403 nucleotides in length. As a non-limiting example, the viral genome comprises a filler region that is about 2405 nucleotides in length. As a non-limiting example, the viral genome comprises a filler region that is about 3013 nucleotides in length. As a non-limiting example, the viral genome comprises a filler region that is about 3021 nucleotides in length.

In one embodiment, the AAV particle viral genome may comprise at least one multiple filler sequence region. The filler region(s) may, independently, have a length such as, but not limited to, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, and 3250 nucleotides. The length of any filler region for the viral genome may be 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, and 3200-3250 nucleotides. As a non-limiting example, the viral genome comprises a filler region that is about 55 nucleotides in length. As a non-limiting example, the viral genome comprises a filler region that is about 56 nucleotides in length. As a non-limiting example, the viral genome comprises a filler region that is about 97 nucleotides in length. As a non-limiting example, the viral genome comprises a filler region that is about 103 nucleotides in length. As a non-limiting example, the viral genome comprises a filler region that is about 105 nucleotides in length. As a non-limiting example, the viral genome comprises a filler region that is about 357 nucleotides in length. As a non-limiting example, the viral genome comprises a filler region that is about 363 nucleotides in length. As a non-limiting example, the viral genome comprises a filler region that is about 712 nucleotides in length. As a non-limiting example, the viral genome comprises a filler region that is about 714 nucleotides in length. As a non-limiting example, the viral genome comprises a filler region that is about 1203 nucleotides in length. As a non-limiting example, the viral genome comprises a filler region that is about 1209 nucleotides in length. As a non-limiting example, the viral genome comprises a filler region that is about 1512 nucleotides in length. As a non-limiting example, the viral genome comprises a filler region that is about 1519 nucleotides in length. As a non-limiting example, the viral genome comprises a filler region that is about 2395 nucleotides in length. As a non-limiting example, the viral genome comprises a filler region that is about 2403 nucleotides in length. As a non-limiting example, the viral genome comprises a filler region that is about 2405 nucleotides in length. As a non-limiting example, the viral genome comprises a filler region that is about 3013 nucleotides in length. As a non-limiting example, the viral genome comprises a filler region that is about 3021 nucleotides in length.

In one embodiment, the AAV particle viral genome comprises at least one filler sequence regions. Non-limiting examples of filler sequence regions are described in Table 19.

TABLE 19
Filler Sequence Regions

| Sequence Region Name | SEQ ID NO |
|---|---|
| FILL1 | 1796 |
| FILL2 | 1797 |
| FILL3 | 1798 |
| FILL4 | 1799 |
| FILL5 | 1800 |
| FILL6 | 1801 |
| FILL7 | 1802 |
| FILL8 | 1803 |
| FILL9 | 1804 |
| FILL10 | 1805 |
| FILL11 | 1806 |
| FILL12 | 1807 |
| FILL13 | 1808 |
| FILL14 | 1809 |
| FILL15 | 1810 |
| FILL16 | 1811 |
| FILL17 | 1812 |
| FILL18 | 1813 |

In one embodiment, the AAV particle viral genome comprises one filler sequence region. In one embodiment, the filler sequence region is the FILL1 sequence region. In one embodiment, the filler sequence region is the FILL2 sequence region. In one embodiment, the filler sequence region is the FILL3 sequence region. In one embodiment, the filler sequence region is the FILL4 sequence region. In one embodiment, the filler sequence region is the FILL5 sequence region. In one embodiment, the filler sequence region is the FILL6 sequence region. In one embodiment, the filler sequence region is the FILL7 sequence region. In one embodiment, the filler sequence region is the FILL8 sequence region. In one embodiment, the filler sequence region is the FILL9 sequence region. In one embodiment, the filler sequence region is the FILL10 sequence region. In one embodiment, the filler sequence region is the FILL11 sequence region. In one embodiment, the filler sequence region is the FILL12 sequence region. In one embodiment, the filler sequence region is the FILL13 sequence region. In one embodiment, the filler sequence region is the FILL14 sequence region. In one embodiment, the filler sequence region is the FILL15 sequence region. In one embodiment, the filler sequence region is the FILL16 sequence region. In one embodiment, the filler sequence region is the FILL17 sequence region. In one embodiment, the filler sequence region is the FILL18 sequence region.

In one embodiment, the AAV particle viral genome comprises two filler sequence regions. In one embodiment, the two filler sequence regions are the FILL1 sequence region, and the FILL2 sequence region. In one embodiment, the filler sequence region is the FILL1 sequence region, and the FILL3 sequence region. In one embodiment, the filler sequence region is the FILL1 sequence region, and the FILL4 sequence region. In one embodiment, the filler sequence region is the FILL1 sequence region, and the FILL5 sequence region. In one embodiment, the filler sequence region is the FILL1 sequence region, and the FILL6 sequence region. In one embodiment, the filler sequence region is the FILL1 sequence region, and the FILL7 sequence region. In one embodiment, the filler sequence region is the FILL1 sequence region, and the FILL8 sequence region. In one embodiment, the filler sequence region is the FILL1 sequence region, and the FILL9 sequence region. In one embodiment, the filler sequence region is the FILL1 sequence region, and the FILL10 sequence region. In one embodiment, the filler sequence region is the FILL1 sequence region, and the FILL11 sequence region. In one embodiment, the filler sequence region is the FILL1 sequence region, and the FILL12 sequence region. In one embodiment, the filler sequence region is the FILL1 sequence region, and the FILL13 sequence region. In one embodiment, the filler sequence region is the FILL1 sequence region, and the FILL14 sequence region. In one embodiment, the filler sequence region is the FILL1 sequence region, and the FILL15 sequence region. In one embodiment, the filler sequence region is the FILL1 sequence region, and the FILL16 sequence region. In one embodiment, the filler sequence region is the FILL1 sequence region, and the FILL17 sequence region. In one embodiment, the filler sequence region is the FILL1 sequence region, and the FILL18 sequence region. In one embodiment, the filler sequence region is the FILL2 sequence region, and the FILL3 sequence region. In one embodiment, the filler sequence region is the FILL3 sequence region, and the FILL4 sequence region. In one embodiment, the filler sequence region is the FILL3 sequence region, and the FILL5 sequence region. In one embodiment, the filler sequence region is the FILL3 sequence region, and the FILL6 sequence region. In one embodiment, the filler sequence region is the FILL3 sequence region, and the FILL7 sequence region. In one embodiment, the filler sequence region is the FILL3 sequence region, and the FILL8 sequence region. In one embodiment, the filler sequence region is the FILL3 sequence region, and the FILL9 sequence region. In one embodiment, the filler sequence region is the FILL3 sequence region, and the FILL10 sequence region. In one embodiment, the filler sequence region is the FILL3 sequence region, and the FILL11 sequence region. In one embodiment, the filler sequence region is the FILL3 sequence region, and the FILL12 sequence region. In one embodiment, the filler sequence region is the FILL3 sequence region, and the FILL13 sequence region. In one embodiment, the filler sequence region is the FILL3 sequence region, and the FILL14 sequence region. In one embodiment, the filler sequence region is the FILL3 sequence region, and the FILL15 sequence region. In one embodiment, the filler sequence region is the FILL3 sequence region, and the FILL16 sequence region. In one embodiment, the filler sequence region is the FILL3 sequence region, and the FILL17 sequence region. In one embodiment, the filler sequence region is the FILL3 sequence region, and the FILL18 sequence region. In one embodiment, the filler sequence region is the FILL4 sequence region, and the FILL5 sequence region. In one embodiment, the filler sequence region is the FILL4 sequence region, and the FILL6 sequence region. In one embodiment, the filler sequence region is the FILL4 sequence region, and the FILL7 sequence region. In one embodiment, the filler sequence region is the FILL4 sequence region, and the FILL8 sequence region. In one embodiment, the filler sequence region is the FILL4 sequence region, and the FILL9 sequence region. In one embodiment, the filler sequence region is the FILL4 sequence region, and the FILL10 sequence region. In one embodiment, the filler sequence region is the FILL4 sequence region, and the FILL11 sequence region. In one embodiment, the filler sequence region is the FILL4 sequence region, and the FILL12 sequence region. In one embodiment, the filler sequence region is the FILL4 sequence region, and the FILL13 sequence region. In one embodiment, the filler sequence region is the FILL4 sequence region, and the FILL14 sequence region. In one embodiment, the filler sequence region is the FILL4 sequence region, and the FILL15 sequence region. In one embodiment, the filler sequence region is the FILL4 sequence region, and the FILL16 sequence region. In one embodiment, the filler sequence region is the FILL4 sequence region, and the FILL17 sequence region. In one embodiment, the filler sequence region is the FILL4 sequence region, and the FILL18 sequence region. In one embodiment, the filler sequence region is the FILL5 sequence region, and the FILL6 sequence region. In one embodiment, the filler sequence region is the FILL5 sequence region, and the FILL7 sequence region. In one embodiment, the filler sequence region is the FILL5 sequence region, and the FILL8 sequence region. In one embodiment, the filler sequence region is the FILL5 sequence region, and the FILL9 sequence region. In one embodiment, the filler sequence region is the FILL5 sequence region, and the FILL10 sequence region. In one embodiment, the filler sequence region is the FILL5 sequence region, and the FILL11 sequence region. In one embodiment, the filler sequence region is the FILL5 sequence region, and the FILL12 sequence region. In one embodiment, the filler sequence region is the FILL5 sequence region, and the FILL13 sequence region. In one embodiment, the filler sequence region is the FILL5 sequence region, and the FILL14 sequence region. In one embodiment, the filler sequence region is the FILL5 sequence region, and the FILL15 sequence region. In one embodiment, the filler sequence region is the FILL5 sequence region, and the FILL16 sequence region. In one embodiment, the filler sequence region is the FILL5 sequence region, and the FILL17 sequence region. In one embodiment, the filler sequence region is the FILL5 sequence region, and the FILL18 sequence region. In one embodiment, the filler sequence region is the FILL6 sequence region, and the FILL7 sequence region. In one embodiment, the filler sequence region is the FILL6 sequence region, and the FILL8 sequence region. In one embodiment, the filler sequence region is the FILL6 sequence region, and the FILL9 sequence region. In one embodiment, the filler sequence region is the FILL6 sequence region, and the FILL10 sequence region. In one embodiment, the filler sequence region is the FILL6 sequence region, and the FILL11 sequence region. In one embodiment, the filler sequence region is the FILL6 sequence region, and the FILL12 sequence region. In one embodiment, the filler sequence region is the FILL6 sequence region, and the FILL13 sequence region. In one embodiment, the filler sequence region is the FILL6 sequence region, and the FILL14 sequence region. In one embodiment, the filler sequence region is the FILL6 sequence region, and the FILL15 sequence region. In one embodiment, the filler sequence region is the FILL6 sequence region, and the FILL16 sequence region. In one embodiment, the filler sequence region is the FILL6 sequence region, and the FILL17 sequence region. In one embodiment, the filler sequence region is the FILL6 sequence region, and the FILL18 sequence region. In one embodiment, the filler sequence region is the FILL7 sequence region, and the FILL8 sequence region. In one embodiment, the filler sequence region is the FILL7 sequence region, and the FILL9 sequence region. In one embodiment, the filler sequence region is the FILL7 sequence region, and the FILL10 sequence region. In one embodiment, the filler sequence region is the FILL7 sequence region, and the FILL11 sequence region. In one embodiment, the filler sequence region is the FILL7 sequence region, and the FILL12 sequence region. In one embodiment, the filler sequence region is the FILL7 sequence region, and the FILL13 sequence region. In one embodiment, the filler sequence region is the FILL7 sequence region, and the FILL14 sequence region. In one embodiment, the filler sequence region is the FILL7 sequence region, and the FILL15 sequence region. In one embodiment, the filler sequence region is the FILL7 sequence region, and the FILL16 sequence region. In one embodiment, the filler sequence region is the FILL7 sequence region, and the FILL17 sequence region. In one embodiment, the filler sequence region is the FILL7 sequence region, and the FILL18 sequence region. In one embodiment, the filler sequence region is the FILL8 sequence region, and the FILL9 sequence region. In one embodiment, the filler sequence region is the FILL8 sequence region, and the FILL10 sequence region. In one embodiment, the filler sequence region is the FILL8 sequence region, and the FILL11 sequence region. In one embodiment, the filler sequence region is the FILL8 sequence region, and the FILL12 sequence region. In one embodiment, the filler sequence region is the FILL8 sequence region, and the FILL13 sequence region. In one embodiment, the filler sequence region is the FILL8 sequence region, and the FILL14 sequence region. In one embodiment, the filler sequence region is the FILL8 sequence region, and the FILL15 sequence region. In one embodiment, the filler sequence region is the FILL8 sequence region, and the FILL16 sequence region. In one embodiment, the filler sequence region is the FILL8 sequence region, and the FILL17 sequence region. In one embodiment, the filler sequence region is the FILL8 sequence region, and the FILL18 sequence region. In one embodiment, the filler sequence region is the FILL9 sequence region, and the FILL10 sequence region. In one embodiment, the filler sequence region is the FILL9 sequence region, and the FILL11 sequence region. In one embodiment, the filler sequence region is the FILL9 sequence region, and the FILL12 sequence region. In one embodiment, the filler sequence region is the FILL9 sequence region, and the FILL13 sequence region. In one embodiment, the filler sequence region is the FILL9 sequence region, and the FILL14 sequence region. In one embodiment, the filler sequence region is the FILL9 sequence region, and the FILL15 sequence region. In one embodiment, the filler sequence region is the FILL9 sequence region, and the FILL16 sequence region. In one embodiment, the filler sequence region is the FILL9 sequence region, and the FILL17 sequence region. In one embodiment, the filler sequence region is the FILL9 sequence region, and the FILL18 sequence region. In one embodiment, the filler sequence region is the FILL10 sequence region, and the FILL11 sequence region. In one embodiment, the filler sequence region is the FILL10 sequence region, and the FILL12 sequence region. In one embodiment, the filler sequence region is the FILL10 sequence region, and the FILL13 sequence region. In one embodiment, the filler sequence region is the FILL10 sequence region, and the FILL14 sequence region. In one embodiment, the filler sequence region is the FILL10 sequence region, and the FILL15 sequence region. In one embodiment, the filler sequence region is the FILL10 sequence region, and the FILL16 sequence region. In one embodiment, the filler sequence region is the FILL10 sequence region, and the FILL17 sequence region. In one embodiment, the filler sequence region is the FILL10 sequence region, and the FILL18 sequence region. In one embodiment, the filler sequence region is the FILL11 sequence region, and the FILL12 sequence region. In one embodiment, the filler sequence region is the FILL11 sequence region, and the FILL13 sequence region. In one embodiment, the filler sequence region is the FILL11 sequence region, and the FILL14 sequence region. In one embodiment, the filler sequence region is the FILL11 sequence region, and the FILL15 sequence region. In one embodiment, the filler sequence region is the FILL11 sequence region, and the FILL16 sequence region. In one embodiment, the filler sequence region is the FILL11 sequence region, and the FILL17 sequence region. In one embodiment, the filler sequence region is the FILL11 sequence region, and the FILL18 sequence region. In one embodiment, the filler sequence region is the FILL12 sequence region, and the FILL13 sequence region. In one embodiment, the filler sequence region is the FILL12 sequence region, and the FILL14 sequence region. In one embodiment, the filler sequence region is the FILL12 sequence region, and the FILL15 sequence region. In one embodiment, the filler sequence region is the FILL12 sequence region, and the FILL16 sequence region. In one embodiment, the filler sequence region is the FILL12 sequence region, and the FILL17 sequence region. In one embodiment, the filler sequence region is the FILL12 sequence region, and the FILL18 sequence region. In one embodiment, the filler sequence region is the FILL13 sequence region, and the FILL14 sequence region. In one embodiment, the filler sequence region is the FILL13 sequence region, and the FILL15 sequence region. In one embodiment, the filler sequence region is the FILL13 sequence region, and the FILL16 sequence region. In one embodiment, the filler sequence region is the FILL13 sequence region, and the FILL17 sequence region. In one embodiment, the filler sequence region is the FILL13 sequence region, and the FILL18 sequence region. In one embodiment, the filler sequence region is the FILL14 sequence region, and the FILL15 sequence region. In one embodiment, the filler sequence region is the FILL14 sequence region, and the FILL16 sequence region. In one embodiment, the filler sequence region is the FILL14 sequence region, and the FILL17 sequence region. In one embodiment, the filler sequence region is the FILL14 sequence region, and the FILL18 sequence region. In one embodiment, the filler sequence region is the FILL15 sequence region, and the FILL16 sequence region. In one embodiment, the filler sequence region is the FILL15 sequence region, and the FILL17 sequence region. In one embodiment, the filler sequence region is the FILL15 sequence region, and the FILL18 sequence region. In one embodiment, the filler sequence region is the FILL16 sequence region, and the FILL17 sequence region. In one embodiment, the filler sequence region is the FILL16 sequence region, and the FILL18 sequence region. In one embodiment, the filler sequence region is the FILL17 sequence region, and the FILL18 sequence region.

In one embodiment, the AAV particle viral genome comprises three filler sequence regions. In one embodiment, the two filler sequence regions are the FILL1 sequence region, the FILL2 sequence region, and the FILL3 sequence region. In one embodiment, the filler sequence region is the FILL1 sequence region, the FILL2 sequence region, and the FILL4 sequence region. In one embodiment, the filler sequence region is the FILL1 sequence region, the FILL2 sequence region, and the FILL5 sequence region. In one embodiment, the filler sequence region is the FILL1 sequence region, the FILL2 sequence region, and the FILL6 sequence region. In one embodiment, the filler sequence region is the FILL1 sequence region, the FILL2 sequence region, and the FILL7 sequence region. In one embodiment, the filler sequence region is the FILL1 sequence region, the FILL2 sequence region, and the FILL8 sequence region. In one embodiment, the filler sequence region is the FILL1 sequence region, the FILL2 sequence region, and the FILL9 sequence region. In one embodiment, the filler sequence region is the FILL1 sequence region, the FILL2 sequence region, and the FILL10 sequence region. In one embodiment, the filler sequence region is the FILL1 sequence region, the FILL2 sequence region, and the FILL11 sequence region. In one embodiment, the filler sequence region is the FILL1 sequence region, the FILL2 sequence region, and the FILL12 sequence region. In one embodiment, the filler sequence region is the FILL1 sequence region, the FILL2 sequence region, and the FILL13 sequence region. In one embodiment, the filler sequence region is the FILL1 sequence region, the FILL2 sequence region, and the FILL14 sequence region. In one embodiment, the filler sequence region is the FILL1 sequence region, the FILL2 sequence region, and the FILL15 sequence region. In one embodiment, the filler sequence region is the FILL1 sequence region, the FILL2 sequence region, and the FILL16 sequence region. In one embodiment, the filler sequence region is the FILL1 sequence region, the FILL2 sequence region, and the FILL17 sequence region. In one embodiment, the filler sequence region is the FILL1 sequence region, the FILL2 sequence region, and the FILL18 sequence region. In one embodiment, the filler sequence region is the FILL1 sequence region, the FILL3 sequence region, and the FILL4 sequence region. In one embodiment, the filler sequence region is the FILL1 sequence region, the FILL3 sequence region, and the FILL5 sequence region. In one embodiment, the filler sequence region is the FILL1 sequence region, the FILL3 sequence region, and the FILL6 sequence region. In one embodiment, the filler sequence region is the FILL1 sequence region, the FILL3 sequence region, and the FILL7 sequence region. In one embodiment, the filler sequence region is the FILL1 sequence region, the FILL3 sequence region, and the FILL8 sequence region. In one embodiment, the filler sequence region is the FILL1 sequence region, the FILL3 sequence region, and the FILL9 sequence region. In one embodiment, the filler sequence region is the FILL1 sequence region, the FILL3 sequence region, and the FILL10 sequence region. In one embodiment, the filler sequence region is the FILL1 sequence region, the FILL3 sequence region, and the FILL11 sequence region. In one embodiment, the filler sequence region is the FILL1 sequence region, the FILL3 sequence region, and the FILL12 sequence region. In one embodiment, the filler sequence region is the FILL1 sequence region, the FILL3 sequence region, and the FILL13 sequence region. In one embodiment, the filler sequence region is the FILL1 sequence region, the FILL3 sequence region, and the FILL14 sequence region. In one embodiment, the filler sequence region is the FILL1 sequence region, the FILL3 sequence region, and the FILL15 sequence region. In one embodiment, the filler sequence region is the FILL1 sequence region, the FILL3 sequence region, and the FILL16 sequence region. In one embodiment, the filler sequence region is the FILL1 sequence region, the FILL3 sequence region, and the FILL17 sequence region. In one embodiment, the filler sequence region is the FILL1 sequence region, the FILL3 sequence region, and the FILL18 sequence region. In one embodiment, the filler sequence region is the FILL1 sequence region, the FILL4 sequence region, and the FILL5 sequence region. In one embodiment, the filler sequence region is the FILL1 sequence region, the FILL4 sequence region, and the FILL6 sequence region. In one embodiment, the filler sequence region is the FILL1 sequence region, the FILL4 sequence region, and the FILL7 sequence region. In one embodiment, the filler sequence region is the FILL1 sequence region, the FILL4 sequence region, and the FILL8 sequence region. In one embodiment, the filler sequence region is the FILL1 sequence region, the FILL4 sequence region, and the FILL9 sequence region. In one embodiment, the filler sequence region is the FILL1 sequence region, the FILL4 sequence region, and the FILL10 sequence region. In one embodiment, the filler sequence region is the FILL1 sequence region, the FILL4 sequence region, and the FILL11 sequence region. In one embodiment, the filler sequence region is the FILL1 sequence region, the FILL4 sequence region, and the FILL12 sequence region. In one embodiment, the filler sequence region is the FILL1 sequence region, the FILL4 sequence region, and the FILL13 sequence region. In one embodiment, the filler sequence region is the FILL1 sequence region, the FILL4 sequence region, and the FILL14 sequence region. In one embodiment, the filler sequence region is the FILL1 sequence region, the FILL4 sequence region, and the FILL15 sequence region. In one embodiment, the filler sequence region is the FILL1 sequence region, the FILL4 sequence region, and the FILL16 sequence region. In one embodiment, the filler sequence region is the FILL1 sequence region, the FILL4 sequence region, and the FILL17 sequence region. In one embodiment, the filler sequence region is the FILL1 sequence region, the FILL4 sequence region, and the FILL18 sequence region. In one embodiment, the filler sequence region is the FILL1 sequence region, the FILL5 sequence region, and the FILL6 sequence region. In one embodiment, the filler sequence region is the FILL1 sequence region, the FILL5 sequence region, and the FILL7 sequence region. In one embodiment, the filler sequence region is the FILL1 sequence region, the FILL5 sequence region, and the FILL8 sequence region. In one embodiment, the filler sequence region is the FILL1 sequence region, the FILL5 sequence region, and the FILL9 sequence region. In one embodiment, the filler sequence region is the FILL1 sequence region, the FILL5 sequence region, and the FILL10 sequence region. In one embodiment, the filler sequence region is the FILL1 sequence region, the FILL5 sequence region, and the FILL11 sequence region. In one embodiment, the filler sequence region is the FILL1 sequence region, the FILL5 sequence region, and the FILL12 sequence region. In one embodiment, the filler sequence region is the FILL1 sequence region, the FILL5 sequence region, and the FILL13 sequence region. In one embodiment, the filler sequence region is the FILL1 sequence region, the FILL5 sequence region, and the FILL14 sequence region. In one embodiment, the filler sequence region is the FILL1 sequence region, the FILL5 sequence region, and the FILL15 sequence region. In one embodiment, the filler sequence region is the FILL1 sequence region, the FILL5 sequence region, and the FILL16 sequence region. In one embodiment, the filler sequence region is the FILL1 sequence region, the FILL5 sequence region, and the FILL17 sequence region. In one embodiment, the filler sequence region is the FILL1 sequence region, the FILL5 sequence region, and the FILL18 sequence region. In one embodiment, the filler sequence region is the FILL1 sequence region, the FILL6 sequence region, and the FILL7 sequence region. In one embodiment, the filler sequence region is the FILL1 sequence region, the FILL6 sequence region, and the FILL8 sequence region. In one embodiment, the filler sequence region is the FILL1 sequence region, the FILL6 sequence region, and the FILL9 sequence region. In one embodiment, the filler sequence region is the FILL1 sequence region, the FILL6 sequence region, and the FILL10 sequence region. In one embodiment, the filler sequence region is the FILL1 sequence region, the FILL6 sequence region, and the FILL11 sequence region. In one embodiment, the filler sequence region is the FILL1 sequence region, the FILL6 sequence region, and the FILL12 sequence region. In one embodiment, the filler sequence region is the FILL1 sequence region, the FILL6 sequence region, and the FILL13 sequence region. In one embodiment, the filler sequence region is the FILL1 sequence region, the FILL6 sequence region, and the FILL14 sequence region. In one embodiment, the filler sequence region is the FILL1 sequence region, the FILL6 sequence region, and the FILL15 sequence region. In one embodiment, the filler sequence region is the FILL1 sequence region, the FILL6 sequence region, and the FILL16 sequence region. In one embodiment, the filler sequence region is the FILL1 sequence region, the FILL6 sequence region, and the FILL17 sequence region. In one embodiment, the filler sequence region is the FILL1 sequence region, the FILL6 sequence region, and the FILL18 sequence region. In one embodiment, the filler sequence region is the FILL1 sequence region, the FILL7 sequence region, and the FILL8 sequence region. In one embodiment, the filler sequence region is the FILL1 sequence region, the FILL7 sequence region, and the FILL9 sequence region. In one embodiment, the filler sequence region is the FILL1 sequence region, the FILL7 sequence region, and the FILL10 sequence region. In one embodiment, the filler sequence region is the FILL1 sequence region, the FILL7 sequence region, and the FILL11 sequence region. In one embodiment, the filler sequence region is the FILL1 sequence region, the FILL7 sequence region, and the FILL12 sequence region. In one embodiment, the filler sequence region is the FILL1 sequence region, the FILL7 sequence region, and the FILL13 sequence region. In one embodiment, the filler sequence region is the FILL1 sequence region, the FILL7 sequence region, and the FILL14 sequence region. In one embodiment, the filler sequence region is the FILL1 sequence region, the FILL7 sequence region, and the FILL15 sequence region. In one embodiment, the filler sequence region is the FILL1 sequence region, the FILL7 sequence region, and the FILL16 sequence region. In one embodiment, the filler sequence region is the FILL1 sequence region, the FILL7 sequence region, and the FILL17 sequence region. In one embodiment, the filler sequence region is the FILL1 sequence region, the FILL7 sequence region, and the FILL18 sequence region. In one embodiment, the filler sequence region is the FILL1 sequence region, the FILL8 sequence region, and the FILL9 sequence region. In one embodiment, the filler sequence region is the FILL1 sequence region, the FILL8 sequence region, and the FILL10 sequence region. In one embodiment, the filler sequence region is the FILL1 sequence region, the FILL8 sequence region, and the FILL11 sequence region. In one embodiment, the filler sequence region is the FILL1 sequence region, the FILL8 sequence region, and the FILL12 sequence region. In one embodiment, the filler sequence region is the FILL1 sequence region, the FILL8 sequence region, and the FILL13 sequence region. In one embodiment, the filler sequence region is the FILL1 sequence region, the FILL8 sequence region, and the FILL14 sequence region. In one embodiment, the filler sequence region is the FILL1 sequence region, the FILL8 sequence region, and the FILL15 sequence region. In one embodiment, the filler sequence region is the FILL1 sequence region, the FILL8 sequence region, and the FILL16 sequence region. In one embodiment, the filler sequence region is the FILL1 sequence region, the FILL8 sequence region, and the FILL17 sequence region. In one embodiment, the filler sequence region is the FILL1 sequence region, the FILL8 sequence region, and the FILL18 sequence region. In one embodiment, the filler sequence region is the FILL1 sequence region, the FILL9 sequence region, and the FILL10 sequence region. In one embodiment, the filler sequence region is the FILL1 sequence region, the FILL9 sequence region, and the FILL11 sequence region. In one embodiment, the filler sequence region is the FILL1 sequence region, the FILL9 sequence region, and the FILL12 sequence region. In one embodiment, the filler sequence region is the FILL1 sequence region, the FILL9 sequence region, and the FILL13 sequence region. In one embodiment, the filler sequence region is the FILL1 sequence region, the FILL9 sequence region, and the FILL14 sequence region. In one embodiment, the filler sequence region is the FILL1 sequence region, the FILL9 sequence region, and the FILL15 sequence region. In one embodiment, the filler sequence region is the FILL1 sequence region, the FILL9 sequence region, and the FILL16 sequence region. In one embodiment, the filler sequence region is the FILL1 sequence region, the FILL9 sequence region, and the FILL17 sequence region. In one embodiment, the filler sequence region is the FILL1 sequence region, the FILL9 sequence region, and the FILL18 sequence region. In one embodiment, the filler sequence region is the FILL1 sequence region, the FILL10 sequence region, and the FILL11 sequence region. In one embodiment, the filler sequence region is the FILL1 sequence region, the FILL10 sequence region, and the FILL12 sequence region. In one embodiment, the filler sequence region is the FILL1 sequence region, the FILL10 sequence region, and the FILL13 sequence region. In one embodiment, the filler sequence region is the FILL1 sequence region, the FILL10 sequence region, and the FILL14 sequence region. In one embodiment, the filler sequence region is the FILL1 sequence region, the FILL10 sequence region, and the FILL15 sequence region. In one embodiment, the filler sequence region is the FILL1 sequence region, the FILL10 sequence region, and the FILL16 sequence region. In one embodiment, the filler sequence region is the FILL1 sequence region, the FILL10 sequence region, and the FILL17 sequence region. In one embodiment, the filler sequence region is the FILL1 sequence region, the FILL10 sequence region, and the FILL18 sequence region. In one embodiment, the filler sequence region is the FILL1 sequence region, the FILL11 sequence region, and the FILL12 sequence region. In one embodiment, the filler sequence region is the FILL1 sequence region, the FILL11 sequence region, and the FILL13 sequence region. In one embodiment, the filler sequence region is the FILL1 sequence region, the FILL11 sequence region, and the FILL14 sequence region. In one embodiment, the filler sequence region is the FILL1 sequence region, the FILL11 sequence region, and the FILL15 sequence region. In one embodiment, the filler sequence region is the FILL1 sequence region, the FILL11 sequence region, and the FILL16 sequence region. In one embodiment, the filler sequence region is the FILL1 sequence region, the FILL11 sequence region, and the FILL17 sequence region. In one embodiment, the filler sequence region is the FILL1 sequence region, the FILL11 sequence region, and the FILL18 sequence region. In one embodiment, the filler sequence region is the FILL1 sequence region, the FILL12 sequence region, and the FILL13 sequence region. In one embodiment, the filler sequence region is the FILL1 sequence region, the FILL12 sequence region, and the FILL14 sequence region. In one embodiment, the filler sequence region is the FILL1 sequence region, the FILL12 sequence region, and the FILL15 sequence region. In one embodiment, the filler sequence region is the FILL1 sequence region, the FILL12 sequence region, and the FILL16 sequence region. In one embodiment, the filler sequence region is the FILL1 sequence region, the FILL12 sequence region, and the FILL17 sequence region. In one embodiment, the filler sequence region is the FILL1 sequence region, the FILL12 sequence region, and the FILL18 sequence region. In one embodiment, the filler sequence region is the FILL1 sequence region, the FILL13 sequence region, and the FILL14 sequence region. In one embodiment, the filler sequence region is the FILL1 sequence region, the FILL13 sequence region, and the FILL15 sequence region. In one embodiment, the filler sequence region is the FILL1 sequence region, the FILL13 sequence region, and the FILL16 sequence region. In one embodiment, the filler sequence region is the FILL1 sequence region, the FILL13 sequence region, and the FILL17 sequence region. In one embodiment, the filler sequence region is the FILL1 sequence region, the FILL13 sequence region, and the FILL18 sequence region. In one embodiment, the filler sequence region is the FILL1 sequence region, the FILL14 sequence region, and the FILL15 sequence region. In one embodiment, the filler sequence region is the FILL1 sequence region, the FILL14 sequence region, and the FILL16 sequence region. In one embodiment, the filler sequence region is the FILL1 sequence region, the FILL14 sequence region, and the FILL17 sequence region. In one embodiment, the filler sequence region is the FILL1 sequence region, the FILL14 sequence region, and the FILL18 sequence region. In one embodiment, the filler sequence region is the FILL1 sequence region, the FILL15 sequence region, and the FILL16 sequence region. In one embodiment, the filler sequence region is the FILL1 sequence region, the FILL15 sequence region, and the FILL17 sequence region. In one embodiment, the filler sequence region is the FILL1 sequence region, the FILL15 sequence region, and the FILL18 sequence region. In one embodiment, the filler sequence region is the FILL1 sequence region, the FILL16 sequence region, and the FILL17 sequence region. In one embodiment, the filler sequence region is the FILL1 sequence region, the FILL16 sequence region, and the FILL18 sequence region. In one embodiment, the filler sequence region is the FILL1 sequence region, the FILL17 sequence region, and the FILL18 sequence region. In one embodiment, the filler sequence region is the FILL2 sequence region, the FILL3 sequence region, and the FILL4 sequence region. In one embodiment, the filler sequence region is the FILL2 sequence region, the FILL3 sequence region, and the FILL5 sequence region. In one embodiment, the filler sequence region is the FILL2 sequence region, the FILL3 sequence region, and the FILL6 sequence region. In one embodiment, the filler sequence region is the FILL2 sequence region, the FILL3 sequence region, and the FILL7 sequence region. In one embodiment, the filler sequence region is the FILL2 sequence region, the FILL3 sequence region, and the FILL8 sequence region. In one embodiment, the filler sequence region is the FILL2 sequence region, the FILL3 sequence region, and the FILL9 sequence region. In one embodiment, the filler sequence region is the FILL2 sequence region, the FILL3 sequence region, and the FILL10 sequence region. In one embodiment, the filler sequence region is the FILL2 sequence region, the FILL3 sequence region, and the FILL11 sequence region. In one embodiment, the filler sequence region is the FILL2 sequence region, the FILL3 sequence region, and the FILL12 sequence region. In one embodiment, the filler sequence region is the FILL2 sequence region, the FILL3 sequence region, and the FILL13 sequence region. In one embodiment, the filler sequence region is the FILL2 sequence region, the FILL3 sequence region, and the FILL14 sequence region. In one embodiment, the filler sequence region is the FILL2 sequence region, the FILL3 sequence region, and the FILL15 sequence region. In one embodiment, the filler sequence region is the FILL2 sequence region, the FILL3 sequence region, and the FILL16 sequence region. In one embodiment, the filler sequence region is the FILL2 sequence region, the FILL3 sequence region, and the FILL17 sequence region. In one embodiment, the filler sequence region is the FILL2 sequence region, the FILL3 sequence region, and the FILL18 sequence region. In one embodiment, the filler sequence region is the FILL2 sequence region, the FILL4 sequence region, and the FILL5 sequence region. In one embodiment, the filler sequence region is the FILL2 sequence region, the FILL4 sequence region, and the FILL6 sequence region. In one embodiment, the filler sequence region is the FILL2 sequence region, the FILL4 sequence region, and the FILL7 sequence region. In one embodiment, the filler sequence region is the FILL2 sequence region, the FILL4 sequence region, and the FILL8 sequence region. In one embodiment, the filler sequence region is the FILL2 sequence region, the FILL4 sequence region, and the FILL9 sequence region. In one embodiment, the filler sequence region is the FILL2 sequence region, the FILL4 sequence region, and the FILL10 sequence region. In one embodiment, the filler sequence region is the FILL2 sequence region, the FILL4 sequence region, and the FILL11 sequence region. In one embodiment, the filler sequence region is the FILL2 sequence region, the FILL4 sequence region, and the FILL12 sequence region. In one embodiment, the filler sequence region is the FILL2 sequence region, the FILL4 sequence region, and the FILL13 sequence region. In one embodiment, the filler sequence region is the FILL2 sequence region, the FILL4 sequence region, and the FILL14 sequence region. In one embodiment, the filler sequence region is the FILL2 sequence region, the FILL4 sequence region, and the FILL15 sequence region. In one embodiment, the filler sequence region is the FILL2 sequence region, the FILL4 sequence region, and the FILL16 sequence region. In one embodiment, the filler sequence region is the FILL2 sequence region, the FILL4 sequence region, and the FILL17 sequence region. In one embodiment, the filler sequence region is the FILL2 sequence region, the FILL4 sequence region, and the FILL18 sequence region. In one embodiment, the filler sequence region is the FILL2 sequence region, the FILL5 sequence region, and the FILL6 sequence region. In one embodiment, the filler sequence region is the FILL2 sequence region, the FILL5 sequence region, and the FILL7 sequence region. In one embodiment, the filler sequence region is the FILL2 sequence region, the FILL5 sequence region, and the FILL8 sequence region. In one embodiment, the filler sequence region is the FILL2 sequence region, the FILL5 sequence region, and the FILL9 sequence region. In one embodiment, the filler sequence region is the FILL2 sequence region, the FILL5 sequence region, and the FILL10 sequence region. In one embodiment, the filler sequence region is the FILL2 sequence region, the FILL5 sequence region, and the FILL11 sequence region. In one embodiment, the filler sequence region is the FILL2 sequence region, the FILL5 sequence region, and the FILL12 sequence region. In one embodiment, the filler sequence region is the FILL2 sequence region, the FILL5 sequence region, and the FILL13 sequence region. In one embodiment, the filler sequence region is the FILL2 sequence region, the FILL5 sequence region, and the FILL14 sequence region. In one embodiment, the filler sequence region is the FILL2 sequence region, the FILL5 sequence region, and the FILL15 sequence region. In one embodiment, the filler sequence region is the FILL2 sequence region, the FILL5 sequence region, and the FILL16 sequence region. In one embodiment, the filler sequence region is the FILL2 sequence region, the FILL5 sequence region, and the FILL17 sequence region. In one embodiment, the filler sequence region is the FILL2 sequence region, the FILL5 sequence region, and the FILL18 sequence region. In one embodiment, the filler sequence region is the FILL2 sequence region, the FILL6 sequence region, and the FILL7 sequence region. In one embodiment, the filler sequence region is the FILL2 sequence region, the FILL6 sequence region, and the FILL8 sequence region. In one embodiment, the filler sequence region is the FILL2 sequence region, the FILL6 sequence region, and the FILL9 sequence region. In one embodiment, the filler sequence region is the FILL2 sequence region, the FILL6 sequence region, and the FILL10 sequence region. In one embodiment, the filler sequence region is the FILL2 sequence region, the FILL6 sequence region, and the FILL11 sequence region. In one embodiment, the filler sequence region is the FILL2 sequence region, the FILL6 sequence region, and the FILL12 sequence region. In one embodiment, the filler sequence region is the FILL2 sequence region, the FILL6 sequence region, and the FILL13 sequence region. In one embodiment, the filler sequence region is the FILL2 sequence region, the FILL6 sequence region, and the FILL14 sequence region. In one embodiment, the filler sequence region is the FILL2 sequence region, the FILL6 sequence region, and the FILL15 sequence region. In one embodiment, the filler sequence region is the FILL2 sequence region, the FILL6 sequence region, and the FILL16 sequence region. In one embodiment, the filler sequence region is the FILL2 sequence region, the FILL6 sequence region, and the FILL17 sequence region. In one embodiment, the filler sequence region is the FILL2 sequence region, the FILL6 sequence region, and the FILL18 sequence region. In one embodiment, the filler sequence region is the FILL2 sequence region, the FILL7 sequence region, and the FILL8 sequence region. In one embodiment, the filler sequence region is the FILL2 sequence region, the FILL7 sequence region, and the FILL9 sequence region. In one embodiment, the filler sequence region is the FILL2 sequence region, the FILL7 sequence region, and the FILL10 sequence region. In one embodiment, the filler sequence region is the FILL2 sequence region, the FILL7 sequence region, and the FILL11 sequence region. In one embodiment, the filler sequence region is the FILL2 sequence region, the FILL7 sequence region, and the FILL12 sequence region. In one embodiment, the filler sequence region is the FILL2 sequence region, the FILL7 sequence region, and the FILL13 sequence region. In one embodiment, the filler sequence region is the FILL2 sequence region, the FILL7 sequence region, and the FILL14 sequence region. In one embodiment, the filler sequence region is the FILL2 sequence region, the FILL7 sequence region, and the FILL15 sequence region. In one embodiment, the filler sequence region is the FILL2 sequence region, the FILL7 sequence region, and the FILL16 sequence region. In one embodiment, the filler sequence region is the FILL2 sequence region, the FILL7 sequence region, and the FILL17 sequence region. In one embodiment, the filler sequence region is the FILL2 sequence region, the FILL7 sequence region, and the FILL18 sequence region. In one embodiment, the filler sequence region is the FILL2 sequence region, the FILL8 sequence region, and the FILL9 sequence region. In one embodiment, the filler sequence region is the FILL2 sequence region, the FILL8 sequence region, and the FILL10 sequence region. In one embodiment, the filler sequence region is the FILL2 sequence region, the FILL8 sequence region, and the FILL11 sequence region. In one embodiment, the filler sequence region is the FILL2 sequence region, the FILL8 sequence region, and the FILL12 sequence region. In one embodiment, the filler sequence region is the FILL2 sequence region, the FILL8 sequence region, and the FILL13 sequence region. In one embodiment, the filler sequence region is the FILL2 sequence region, the FILL8 sequence region, and the FILL14 sequence region. In one embodiment, the filler sequence region is the FILL2 sequence region, the FILL8 sequence region, and the FILL15 sequence region. In one embodiment, the filler sequence region is the FILL2 sequence region, the FILL8 sequence region, and the FILL16 sequence region. In one embodiment, the filler sequence region is the FILL2 sequence region, the FILL8 sequence region, and the FILL17 sequence region. In one embodiment, the filler sequence region is the FILL2 sequence region, the FILL8 sequence region, and the FILL18 sequence region. In one embodiment, the filler sequence region is the FILL2 sequence region, the FILL9 sequence region, and the FILL10 sequence region. In one embodiment, the filler sequence region is the FILL2 sequence region, the FILL9 sequence region, and the FILL11 sequence region. In one embodiment, the filler sequence region is the FILL2 sequence region, the FILL9 sequence region, and the FILL12 sequence region. In one embodiment, the filler sequence region is the FILL2 sequence region, the FILL9 sequence region, and the FILL13 sequence region. In one embodiment, the filler sequence region is the FILL2 sequence region, the FILL9 sequence region, and the FILL14 sequence region. In one embodiment, the filler sequence region is the FILL2 sequence region, the FILL9 sequence region, and the FILL15 sequence region. In one embodiment, the filler sequence region is the FILL2 sequence region, the FILL9 sequence region, and the FILL16 sequence region. In one embodiment, the filler sequence region is the FILL2 sequence region, the FILL9 sequence region, and the FILL17 sequence region. In one embodiment, the filler sequence region is the FILL2 sequence region, the FILL9 sequence region, and the FILL18 sequence region. In one embodiment, the filler sequence region is the FILL2 sequence region, the FILL10 sequence region, and the FILL11 sequence region. In one embodiment, the filler sequence region is the FILL2 sequence region, the FILL10 sequence region, and the FILL12 sequence region. In one embodiment, the filler sequence region is the FILL2 sequence region, the FILL10 sequence region, and the FILL13 sequence region. In one embodiment, the filler sequence region is the FILL2 sequence region, the FILL10 sequence region, and the FILL14 sequence region. In one embodiment, the filler sequence region is the FILL2 sequence region, the FILL10 sequence region, and the FILL15 sequence region. In one embodiment, the filler sequence region is the FILL2 sequence region, the FILL10 sequence region, and the FILL16 sequence region. In one embodiment, the filler sequence region is the FILL2 sequence region, the FILL10 sequence region, and the FILL17 sequence region. In one embodiment, the filler sequence region is the FILL2 sequence region, the FILL10 sequence region, and the FILL18 sequence region. In one embodiment, the filler sequence region is the FILL2 sequence region, the FILL11 sequence region, and the FILL12 sequence region. In one embodiment, the filler sequence region is the FILL2 sequence region, the FILL11 sequence region, and the FILL13 sequence region. In one embodiment, the filler sequence region is the FILL2 sequence region, the FILL11 sequence region, and the FILL14 sequence region. In one embodiment, the filler sequence region is the FILL2 sequence region, the FILL11 sequence region, and the FILL15 sequence region. In one embodiment, the filler sequence region is the FILL2 sequence region, the FILL11 sequence region, and the FILL16 sequence region. In one embodiment, the filler sequence region is the FILL2 sequence region, the FILL11 sequence region, and the FILL17 sequence region. In one embodiment, the filler sequence region is the FILL2 sequence region, the FILL11 sequence region, and the FILL18 sequence region. In one embodiment, the filler sequence region is the FILL2 sequence region, the FILL12 sequence region, and the FILL13 sequence region. In one embodiment, the filler sequence region is the FILL2 sequence region, the FILL12 sequence region, and the FILL14 sequence region. In one embodiment, the filler sequence region is the FILL2 sequence region, the FILL12 sequence region, and the FILL15 sequence region. In one embodiment, the filler sequence region is the FILL2 sequence region, the FILL12 sequence region, and the FILL16 sequence region. In one embodiment, the filler sequence region is the FILL2 sequence region, the FILL12 sequence region, and the FILL17 sequence region. In one embodiment, the filler sequence region is the FILL2 sequence region, the FILL12 sequence region, and the FILL18 sequence region. In one embodiment, the filler sequence region is the FILL2 sequence region, the FILL13 sequence region, and the FILL14 sequence region. In one embodiment, the filler sequence region is the FILL2 sequence region, the FILL13 sequence region, and the FILL15 sequence region. In one embodiment, the filler sequence region is the FILL2 sequence region, the FILL13 sequence region, and the FILL16 sequence region. In one embodiment, the filler sequence region is the FILL2 sequence region, the FILL13 sequence region, and the FILL17 sequence region. In one embodiment, the filler sequence region is the FILL2 sequence region, the FILL13 sequence region, and the FILL18 sequence region. In one embodiment, the filler sequence region is the FILL2 sequence region, the FILL14 sequence region, and the FILL15 sequence region. In one embodiment, the filler sequence region is the FILL2 sequence region, the FILL14 sequence region, and the FILL16 sequence region. In one embodiment, the filler sequence region is the FILL2 sequence region, the FILL14 sequence region, and the FILL17 sequence region. In one embodiment, the filler sequence region is the FILL2 sequence region, the FILL14 sequence region, and the FILL18 sequence region. In one embodiment, the filler sequence region is the FILL2 sequence region, the FILL15 sequence region, and the FILL16 sequence region. In one embodiment, the filler sequence region is the FILL2 sequence region, the FILL15 sequence region, and the FILL17 sequence region. In one embodiment, the filler sequence region is the FILL2 sequence region, the FILL15 sequence region, and the FILL18 sequence region. In one embodiment, the filler sequence region is the FILL2 sequence region, the FILL16 sequence region, and the FILL17 sequence region. In one embodiment, the filler sequence region is the FILL2 sequence region, the FILL16 sequence region, and the FILL18 sequence region. In one embodiment, the filler sequence region is the FILL2 sequence region, the FILL17 sequence region, and the FILL18 sequence region. In one embodiment, the filler sequence region is the FILL3 sequence region, the FILL4 sequence region, and the FILL5 sequence region. In one embodiment, the filler sequence region is the FILL3 sequence region, the FILL4 sequence region, and the FILL6 sequence region. In one embodiment, the filler sequence region is the FILL3 sequence region, the FILL4 sequence region, and the FILL7 sequence region. In one embodiment, the filler sequence region is the FILL3 sequence region, the FILL4 sequence region, and the FILL8 sequence region. In one embodiment, the filler sequence region is the FILL3 sequence region, the FILL4 sequence region, and the FILL9 sequence region. In one embodiment, the filler sequence region is the FILL3 sequence region, the FILL4 sequence region, and the FILL10 sequence region. In one embodiment, the filler sequence region is the FILL3 sequence region, the FILL4 sequence region, and the FILL11 sequence region. In one embodiment, the filler sequence region is the FILL3 sequence region, the FILL4 sequence region, and the FILL12 sequence region. In one embodiment, the filler sequence region is the FILL3 sequence region, the FILL4 sequence region, and the FILL13 sequence region. In one embodiment, the filler sequence region is the FILL3 sequence region, the FILL4 sequence region, and the FILL14 sequence region. In one embodiment, the filler sequence region is the FILL3 sequence region, the FILL4 sequence region, and the FILL15 sequence region. In one embodiment, the filler sequence region is the FILL3 sequence region, the FILL4 sequence region, and the FILL16 sequence region. In one embodiment, the filler sequence region is the FILL3 sequence region, the FILL4 sequence region, and the FILL17 sequence region. In one embodiment, the filler sequence region is the FILL3 sequence region, the FILL4 sequence region, and the FILL18 sequence region. In one embodiment, the filler sequence region is the FILL3 sequence region, the FILL5 sequence region, and the FILL6 sequence region. In one embodiment, the filler sequence region is the FILL3 sequence region, the FILL5 sequence region, and the FILL7 sequence region. In one embodiment, the filler sequence region is the FILL3 sequence region, the FILL5 sequence region, and the FILL8 sequence region. In one embodiment, the filler sequence region is the FILL3 sequence region, the FILL5 sequence region, and the FILL9 sequence region. In one embodiment, the filler sequence region is the FILL3 sequence region, the FILL5 sequence region, and the FILL10 sequence region. In one embodiment, the filler sequence region is the FILL3 sequence region, the FILL5 sequence region, and the FILL11 sequence region. In one embodiment, the filler sequence region is the FILL3 sequence region, the FILL5 sequence region, and the FILL12 sequence region. In one embodiment, the filler sequence region is the FILL3 sequence region, the FILL5 sequence region, and the FILL13 sequence region. In one embodiment, the filler sequence region is the FILL3 sequence region, the FILL5 sequence region, and the FILL14 sequence region. In one embodiment, the filler sequence region is the FILL3 sequence region, the FILL5 sequence region, and the FILL15 sequence region. In one embodiment, the filler sequence region is the FILL3 sequence region, the FILL5 sequence region, and the FILL16 sequence region. In one embodiment, the filler sequence region is the FILL3 sequence region, the FILL5 sequence region, and the FILL17 sequence region. In one embodiment, the filler sequence region is the FILL3 sequence region, the FILL5 sequence region, and the FILL18 sequence region. In one embodiment, the filler sequence region is the FILL3 sequence region, the FILL6 sequence region, and the FILL7 sequence region. In one embodiment, the filler sequence region is the FILL3 sequence region, the FILL6 sequence region, and the FILL8 sequence region. In one embodiment, the filler sequence region is the FILL3 sequence region, the FILL6 sequence region, and the FILL9 sequence region. In one embodiment, the filler sequence region is the FILL3 sequence region, the FILL6 sequence region, and the FILL10 sequence region. In one embodiment, the filler sequence region is the FILL3 sequence region, the FILL6 sequence region, and the FILL11 sequence region. In one embodiment, the filler sequence region is the FILL3 sequence region, the FILL6 sequence region, and the FILL12 sequence region. In one embodiment, the filler sequence region is the FILL3 sequence region, the FILL6 sequence region, and the FILL13 sequence region. In one embodiment, the filler sequence region is the FILL3 sequence region, the FILL6 sequence region, and the FILL14 sequence region. In one embodiment, the filler sequence region is the FILL3 sequence region, the FILL6 sequence region, and the FILL15 sequence region. In one embodiment, the filler sequence region is the FILL3 sequence region, the FILL6 sequence region, and the FILL16 sequence region. In one embodiment, the filler sequence region is the FILL3 sequence region, the FILL6 sequence region, and the FILL17 sequence region. In one embodiment, the filler sequence region is the FILL3 sequence region, the FILL6 sequence region, and the FILL18 sequence region. In one embodiment, the filler sequence region is the FILL3 sequence region, the FILL7 sequence region, and the FILL8 sequence region. In one embodiment, the filler sequence region is the FILL3 sequence region, the FILL7 sequence region, and the FILL9 sequence region. In one embodiment, the filler sequence region is the FILL3 sequence region, the FILL7 sequence region, and the FILL10 sequence region. In one embodiment, the filler sequence region is the FILL3 sequence region, the FILL7 sequence region, and the FILL11 sequence region. In one embodiment, the filler sequence region is the FILL3 sequence region, the FILL7 sequence region, and the FILL12 sequence region. In one embodiment, the filler sequence region is the FILL3 sequence region, the FILL7 sequence region, and the FILL13 sequence region. In one embodiment, the filler sequence region is the FILL3 sequence region, the FILL7 sequence region, and the FILL14 sequence region. In one embodiment, the filler sequence region is the FILL3 sequence region, the FILL7 sequence region, and the FILL15 sequence region. In one embodiment, the filler sequence region is the FILL3 sequence region, the FILL7 sequence region, and the FILL16 sequence region. In one embodiment, the filler sequence region is the FILL3 sequence region, the FILL7 sequence region, and the FILL17 sequence region. In one embodiment, the filler sequence region is the FILL3 sequence region, the FILL7 sequence region, and the FILL18 sequence region. In one embodiment, the filler sequence region is the FILL3 sequence region, the FILL8 sequence region, and the FILL9 sequence region. In one embodiment, the filler sequence region is the FILL3 sequence region, the FILL8 sequence region, and the FILL10 sequence region. In one embodiment, the filler sequence region is the FILL3 sequence region, the FILL8 sequence region, and the FILL11 sequence region. In one embodiment, the filler sequence region is the FILL3 sequence region, the FILL8 sequence region, and the FILL12 sequence region. In one embodiment, the filler sequence region is the FILL3 sequence region, the FILL8 sequence region, and the FILL13 sequence region. In one embodiment, the filler sequence region is the FILL3 sequence region, the FILL8 sequence region, and the FILL14 sequence region. In one embodiment, the filler sequence region is the FILL3 sequence region, the FILL8 sequence region, and the FILL15 sequence region. In one embodiment, the filler sequence region is the FILL3 sequence region, the FILL8 sequence region, and the FILL16 sequence region. In one embodiment, the filler sequence region is the FILL3 sequence region, the FILL8 sequence region, and the FILL17 sequence region. In one embodiment, the filler sequence region is the FILL3 sequence region, the FILL8 sequence region, and the FILL18 sequence region. In one embodiment, the filler sequence region is the FILL3 sequence region, the FILL9 sequence region, and the FILL10 sequence region. In one embodiment, the filler sequence region is the FILL3 sequence region, the FILL9 sequence region, and the FILL11 sequence region. In one embodiment, the filler sequence region is the FILL3 sequence region, the FILL9 sequence region, and the FILL12 sequence region. In one embodiment, the filler sequence region is the FILL3 sequence region, the FILL9 sequence region, and the FILL13 sequence region. In one embodiment, the filler sequence region is the FILL3 sequence region, the FILL9 sequence region, and the FILL14 sequence region. In one embodiment, the filler sequence region is the FILL3 sequence region, the FILL9 sequence region, and the FILL15 sequence region. In one embodiment, the filler sequence region is the FILL3 sequence region, the FILL9 sequence region, and the FILL16 sequence region. In one embodiment, the filler sequence region is the FILL3 sequence region, the FILL9 sequence region, and the FILL17 sequence region. In one embodiment, the filler sequence region is the FILL3 sequence region, the FILL9 sequence region, and the FILL18 sequence region. In one embodiment, the filler sequence region is the FILL3 sequence region, the FILL10 sequence region, and the FILL11 sequence region. In one embodiment, the filler sequence region is the FILL3 sequence region, the FILL10 sequence region, and the FILL12 sequence region. In one embodiment, the filler sequence region is the FILL3 sequence region, the FILL10 sequence region, and the FILL13 sequence region. In one embodiment, the filler sequence region is the FILL3 sequence region, the FILL10 sequence region, and the FILL14 sequence region. In one embodiment, the filler sequence region is the FILL3 sequence region, the FILL10 sequence region, and the FILL15 sequence region. In one embodiment, the filler sequence region is the FILL3 sequence region, the FILL10 sequence region, and the FILL16 sequence region. In one embodiment, the filler sequence region is the FILL3 sequence region, the FILL10 sequence region, and the FILL17 sequence region. In one embodiment, the filler sequence region is the FILL3 sequence region, the FILL10 sequence region, and the FILL18 sequence region. In one embodiment, the filler sequence region is the FILL3 sequence region, the FILL11 sequence region, and the FILL12 sequence region. In one embodiment, the filler sequence region is the FILL3 sequence region, the FILL11 sequence region, and the FILL13 sequence region. In one embodiment, the filler sequence region is the FILL3 sequence region, the FILL11 sequence region, and the FILL14 sequence region. In one embodiment, the filler sequence region is the FILL3 sequence region, the FILL11 sequence region, and the FILL15 sequence region. In one embodiment, the filler sequence region is the FILL3 sequence region, the FILL11 sequence region, and the FILL16 sequence region. In one embodiment, the filler sequence region is the FILL3 sequence region, the FILL11 sequence region, and the FILL17 sequence region. In one embodiment, the filler sequence region is the FILL3 sequence region, the FILL11 sequence region, and the FILL18 sequence region. In one embodiment, the filler sequence region is the FILL3 sequence region, the FILL12 sequence region, and the FILL13 sequence region. In one embodiment, the filler sequence region is the FILL3 sequence region, the FILL12 sequence region, and the FILL14 sequence region. In one embodiment, the filler sequence region is the FILL3 sequence region, the FILL12 sequence region, and the FILL15 sequence region. In one embodiment, the filler sequence region is the FILL3 sequence region, the FILL12 sequence region, and the FILL16 sequence region. In one embodiment, the filler sequence region is the FILL3 sequence region, the FILL12 sequence region, and the FILL17 sequence region. In one embodiment, the filler sequence region is the FILL3 sequence region, the FILL12 sequence region, and the FILL18 sequence region. In one embodiment, the filler sequence region is the FILL3 sequence region, the FILL13 sequence region, and the FILL14 sequence region. In one embodiment, the filler sequence region is the FILL3 sequence region, the FILL13 sequence region, and the FILL15 sequence region. In one embodiment, the filler sequence region is the FILL3 sequence region, the FILL13 sequence region, and the FILL16 sequence region. In one embodiment, the filler sequence region is the FILL3 sequence region, the FILL13 sequence region, and the FILL17 sequence region. In one embodiment, the filler sequence region is the FILL3 sequence region, the FILL13 sequence region, and the FILL18 sequence region. In one embodiment, the filler sequence region is the FILL3 sequence region, the FILL14 sequence region, and the FILL15 sequence region. In one embodiment, the filler sequence region is the FILL3 sequence region, the FILL14 sequence region, and the FILL16 sequence region. In one embodiment, the filler sequence region is the FILL3 sequence region, the FILL14 sequence region, and the FILL17 sequence region. In one embodiment, the filler sequence region is the FILL3 sequence region, the FILL14 sequence region, and the FILL18 sequence region. In one embodiment, the filler sequence region is the FILL3 sequence region, the FILL15 sequence region, and the FILL16 sequence region. In one embodiment, the filler sequence region is the FILL3 sequence region, the FILL15 sequence region, and the FILL17 sequence region. In one embodiment, the filler sequence region is the FILL3 sequence region, the FILL15 sequence region, and the FILL18 sequence region. In one embodiment, the filler sequence region is the FILL3 sequence region, the FILL16 sequence region, and the FILL17 sequence region. In one embodiment, the filler sequence region is the FILL3 sequence region, the FILL16 sequence region, and the FILL18 sequence region. In one embodiment, the filler sequence region is the FILL3 sequence region, the FILL17 sequence region, and the FILL18 sequence region. In one embodiment, the filler sequence region is the FILL4 sequence region, the FILL5 sequence region, and the FILL6 sequence region. In one embodiment, the filler sequence region is the FILL4 sequence region, the FILL5 sequence region, and the FILL7 sequence region. In one embodiment, the filler sequence region is the FILL4 sequence region, the FILL5 sequence region, and the FILL8 sequence region. In one embodiment, the filler sequence region is the FILL4 sequence region, the FILL5 sequence region, and the FILL9 sequence region. In one embodiment, the filler sequence region is the FILL4 sequence region, the FILL5 sequence region, and the FILL10 sequence region. In one embodiment, the filler sequence region is the FILL4 sequence region, the FILL5 sequence region, and the FILL11 sequence region. In one embodiment, the filler sequence region is the FILL4 sequence region, the FILL5 sequence region, and the FILL12 sequence region. In one embodiment, the filler sequence region is the FILL4 sequence region, the FILL5 sequence region, and the FILL13 sequence region. In one embodiment, the filler sequence region is the FILL4 sequence region, the FILL5 sequence region, and the FILL14 sequence region. In one embodiment, the filler sequence region is the FILL4 sequence region, the FILL5 sequence region, and the FILL15 sequence region. In one embodiment, the filler sequence region is the FILL4 sequence region, the FILL5 sequence region, and the FILL16 sequence region. In one embodiment, the filler sequence region is the FILL4 sequence region, the FILL5 sequence region, and the FILL17 sequence region. In one embodiment, the filler sequence region is the FILL4 sequence region, the FILL5 sequence region, and the FILL18 sequence region. In one embodiment, the filler sequence region is the FILL4 sequence region, the FILL6 sequence region, and the FILL7 sequence region. In one embodiment, the filler sequence region is the FILL4 sequence region, the FILL6 sequence region, and the FILL8 sequence region. In one embodiment, the filler sequence region is the FILL4 sequence region, the FILL6 sequence region, and the FILL9 sequence region. In one embodiment, the filler sequence region is the FILL4 sequence region, the FILL6 sequence region, and the FILL10 sequence region. In one embodiment, the filler sequence region is the FILL4 sequence region, the FILL6 sequence region, and the FILL11 sequence region. In one embodiment, the filler sequence region is the FILL4 sequence region, the FILL6 sequence region, and the FILL12 sequence region. In one embodiment, the filler sequence region is the FILL4 sequence region, the FILL6 sequence region, and the FILL13 sequence region. In one embodiment, the filler sequence region is the FILL4 sequence region, the FILL6 sequence region, and the FILL14 sequence region. In one embodiment, the filler sequence region is the FILL4 sequence region, the FILL6 sequence region, and the FILL15 sequence region. In one embodiment, the filler sequence region is the FILL4 sequence region, the FILL6 sequence region, and the FILL16 sequence region. In one embodiment, the filler sequence region is the FILL4 sequence region, the FILL6 sequence region, and the FILL17 sequence region. In one embodiment, the filler sequence region is the FILL4 sequence region, the FILL6 sequence region, and the FILL18 sequence region. In one embodiment, the filler sequence region is the FILL4 sequence region, the FILL7 sequence region, and the FILL8 sequence region. In one embodiment, the filler sequence region is the FILL4 sequence region, the FILL7 sequence region, and the FILL9 sequence region. In one embodiment, the filler sequence region is the FILL4 sequence region, the FILL7 sequence region, and the FILL10 sequence region. In one embodiment, the filler sequence region is the FILL4 sequence region, the FILL7 sequence region, and the FILL11 sequence region. In one embodiment, the filler sequence region is the FILL4 sequence region, the FILL7 sequence region, and the FILL12 sequence region. In one embodiment, the filler sequence region is the FILL4 sequence region, the FILL7 sequence region, and the FILL13 sequence region. In one embodiment, the filler sequence region is the FILL4 sequence region, the FILL7 sequence region, and the FILL14 sequence region. In one embodiment, the filler sequence region is the FILL4 sequence region, the FILL7 sequence region, and the FILL15 sequence region. In one embodiment, the filler sequence region is the FILL4 sequence region, the FILL7 sequence region, and the FILL16 sequence region. In one embodiment, the filler sequence region is the FILL4 sequence region, the FILL7 sequence region, and the FILL17 sequence region. In one embodiment, the filler sequence region is the FILL4 sequence region, the FILL7 sequence region, and the FILL18 sequence region. In one embodiment, the filler sequence region is the FILL4 sequence region, the FILL8 sequence region, and the FILL9 sequence region. In one embodiment, the filler sequence region is the FILL4 sequence region, the FILL8 sequence region, and the FILL10 sequence region. In one embodiment, the filler sequence region is the FILL4 sequence region, the FILL8 sequence region, and the FILL11 sequence region. In one embodiment, the filler sequence region is the FILL4 sequence region, the FILL8 sequence region, and the FILL12 sequence region. In one embodiment, the filler sequence region is the FILL4 sequence region, the FILL8 sequence region, and the FILL13 sequence region. In one embodiment, the filler sequence region is the FILL4 sequence region, the FILL8 sequence region, and the FILL14 sequence region. In one embodiment, the filler sequence region is the FILL4 sequence region, the FILL8 sequence region, and the FILL15 sequence region. In one embodiment, the filler sequence region is the FILL4 sequence region, the FILL8 sequence region, and the FILL16 sequence region. In one embodiment, the filler sequence region is the FILL4 sequence region, the FILL8 sequence region, and the FILL17 sequence region. In one embodiment, the filler sequence region is the FILL4 sequence region, the FILL8 sequence region, and the FILL18 sequence region. In one embodiment, the filler sequence region is the FILL4 sequence region, the FILL9 sequence region, and the FILL10 sequence region. In one embodiment, the filler sequence region is the FILL4 sequence region, the FILL9 sequence region, and the FILL11 sequence region. In one embodiment, the filler sequence region is the FILL4 sequence region, the FILL9 sequence region, and the FILL12 sequence region. In one embodiment, the filler sequence region is the FILL4 sequence region, the FILL9 sequence region, and the FILL13 sequence region. In one embodiment, the filler sequence region is the FILL4 sequence region, the FILL9 sequence region, and the FILL14 sequence region. In one embodiment, the filler sequence region is the FILL4 sequence region, the FILL9 sequence region, and the FILL15 sequence region. In one embodiment, the filler sequence region is the FILL4 sequence region, the FILL9 sequence region, and the FILL16 sequence region. In one embodiment, the filler sequence region is the FILL4 sequence region, the FILL9 sequence region, and the FILL17 sequence region. In one embodiment, the filler sequence region is the FILL4 sequence region, the FILL9 sequence region, and the FILL18 sequence region. In one embodiment, the filler sequence region is the FILL4 sequence region, the FILL10 sequence region, and the FILL11 sequence region. In one embodiment, the filler sequence region is the FILL4 sequence region, the FILL10 sequence region, and the FILL12 sequence region. In one embodiment, the filler sequence region is the FILL4 sequence region, the FILL10 sequence region, and the FILL13 sequence region. In one embodiment, the filler sequence region is the FILL4 sequence region, the FILL10 sequence region, and the FILL14 sequence region. In one embodiment, the filler sequence region is the FILL4 sequence region, the FILL10 sequence region, and the FILL15 sequence region. In one embodiment, the filler sequence region is the FILL4 sequence region, the FILL10 sequence region, and the FILL16 sequence region. In one embodiment, the filler sequence region is the FILL4 sequence region, the FILL10 sequence region, and the FILL17 sequence region. In one embodiment, the filler sequence region is the FILL4 sequence region, the FILL10 sequence region, and the FILL18 sequence region. In one embodiment, the filler sequence region is the FILL4 sequence region, the FILL11 sequence region, and the FILL12 sequence region. In one embodiment, the filler sequence region is the FILL4 sequence region, the FILL11 sequence region, and the FILL13 sequence region. In one embodiment, the filler sequence region is the FILL4 sequence region, the FILL11 sequence region, and the FILL14 sequence region. In one embodiment, the filler sequence region is the FILL4 sequence region, the FILL11 sequence region, and the FILL15 sequence region. In one embodiment, the filler sequence region is the FILL4 sequence region, the FILL11 sequence region, and the FILL16 sequence region. In one embodiment, the filler sequence region is the FILL4 sequence region, the FILL11 sequence region, and the FILL17 sequence region. In one embodiment, the filler sequence region is the FILL4 sequence region, the FILL11 sequence region, and the FILL18 sequence region. In one embodiment, the filler sequence region is the FILL4 sequence region, the FILL12 sequence region, and the FILL13 sequence region. In one embodiment, the filler sequence region is the FILL4 sequence region, the FILL12 sequence region, and the FILL14 sequence region. In one embodiment, the filler sequence region is the FILL4 sequence region, the FILL12 sequence region, and the FILL15 sequence region. In one embodiment, the filler sequence region is the FILL4 sequence region, the FILL12 sequence region, and the FILL16 sequence region. In one embodiment, the filler sequence region is the FILL4 sequence region, the FILL12 sequence region, and the FILL17 sequence region. In one embodiment, the filler sequence region is the FILL4 sequence region, the FILL12 sequence region, and the FILL18 sequence region. In one embodiment, the filler sequence region is the FILL4 sequence region, the FILL13 sequence region, and the FILL14 sequence region. In one embodiment, the filler sequence region is the FILL4 sequence region, the FILL13 sequence region, and the FILL15 sequence region. In one embodiment, the filler sequence region is the FILL4 sequence region, the FILL13 sequence region, and the FILL16 sequence region. In one embodiment, the filler sequence region is the FILL4 sequence region, the FILL13 sequence region, and the FILL17 sequence region. In one embodiment, the filler sequence region is the FILL4 sequence region, the FILL13 sequence region, and the FILL18 sequence region. In one embodiment, the filler sequence region is the FILL4 sequence region, the FILL14 sequence region, and the FILL15 sequence region. In one embodiment, the filler sequence region is the FILL4 sequence region, the FILL14 sequence region, and the FILL16 sequence region. In one embodiment, the filler sequence region is the FILL4 sequence region, the FILL14 sequence region, and the FILL17 sequence region. In one embodiment, the filler sequence region is the FILL4 sequence region, the FILL14 sequence region, and the FILL18 sequence region. In one embodiment, the filler sequence region is the FILL4 sequence region, the FILL15 sequence region, and the FILL16 sequence region. In one embodiment, the filler sequence region is the FILL4 sequence region, the FILL15 sequence region, and the FILL17 sequence region. In one embodiment, the filler sequence region is the FILL4 sequence region, the FILL15 sequence region, and the FILL18 sequence region. In one embodiment, the filler sequence region is the FILL4 sequence region, the FILL16 sequence region, and the FILL17 sequence region. In one embodiment, the filler sequence region is the FILL4 sequence region, the FILL16 sequence region, and the FILL18 sequence region. In one embodiment, the filler sequence region is the FILL4 sequence region, the FILL17 sequence region, and the FILL18 sequence region. In one embodiment, the filler sequence region is the FILL5 sequence region, the FILL6 sequence region, and the FILL7 sequence region. In one embodiment, the filler sequence region is the FILL5 sequence region, the FILL6 sequence region, and the FILL8 sequence region. In one embodiment, the filler sequence region is the FILL5 sequence region, the FILL6 sequence region, and the FILL9 sequence region. In one embodiment, the filler sequence region is the FILL5 sequence region, the FILL6 sequence region, and the FILL10 sequence region. In one embodiment, the filler sequence region is the FILL5 sequence region, the FILL6 sequence region, and the FILL11 sequence region. In one embodiment, the filler sequence region is the FILL5 sequence region, the FILL6 sequence region, and the FILL12 sequence region. In one embodiment, the filler sequence region is the FILL5 sequence region, the FILL6 sequence region, and the FILL13 sequence region. In one embodiment, the filler sequence region is the FILL5 sequence region, the FILL6 sequence region, and the FILL14 sequence region. In one embodiment, the filler sequence region is the FILL5 sequence region, the FILL6 sequence region, and the FILL15 sequence region. In one embodiment, the filler sequence region is the FILL5 sequence region, the FILL6 sequence region, and the FILL16 sequence region. In one embodiment, the filler sequence region is the FILL5 sequence region, the FILL6 sequence region, and the FILL17 sequence region. In one embodiment, the filler sequence region is the FILL5 sequence region, the FILL6 sequence region, and the FILL18 sequence region. In one embodiment, the filler sequence region is the FILL5 sequence region, the FILL7 sequence region, and the FILL8 sequence region. In one embodiment, the filler sequence region is the FILL5 sequence region, the FILL7 sequence region, and the FILL9 sequence region. In one embodiment, the filler sequence region is the FILL5 sequence region, the FILL7 sequence region, and the FILL10 sequence region. In one embodiment, the filler sequence region is the FILL5 sequence region, the FILL7 sequence region, and the FILL11 sequence region. In one embodiment, the filler sequence region is the FILL5 sequence region, the FILL7 sequence region, and the FILL12 sequence region. In one embodiment, the filler sequence region is the FILL5 sequence region, the FILL7 sequence region, and the FILL13 sequence region. In one embodiment, the filler sequence region is the FILL5 sequence region, the FILL7 sequence region, and the FILL14 sequence region. In one embodiment, the filler sequence region is the FILL5 sequence region, the FILL7 sequence region, and the FILL15 sequence region. In one embodiment, the filler sequence region is the FILL5 sequence region, the FILL7 sequence region, and the FILL16 sequence region. In one embodiment, the filler sequence region is the FILL5 sequence region, the FILL7 sequence region, and the FILL17 sequence region. In one embodiment, the filler sequence region is the FILL5 sequence region, the FILL7 sequence region, and the FILL18 sequence region. In one embodiment, the filler sequence region is the FILL5 sequence region, the FILL8 sequence region, and the FILL9 sequence region. In one embodiment, the filler sequence region is the FILL5 sequence region, the FILL8 sequence region, and the FILL10 sequence region. In one embodiment, the filler sequence region is the FILL5 sequence region, the FILL8 sequence region, and the FILL11 sequence region. In one embodiment, the filler sequence region is the FILL5 sequence region, the FILL8 sequence region, and the FILL12 sequence region. In one embodiment, the filler sequence region is the FILL5 sequence region, the FILL8 sequence region, and the FILL13 sequence region. In one embodiment, the filler sequence region is the FILL5 sequence region, the FILL8 sequence region, and the FILL14 sequence region. In one embodiment, the filler sequence region is the FILL5 sequence region, the FILL8 sequence region, and the FILL15 sequence region. In one embodiment, the filler sequence region is the FILL5 sequence region, the FILL8 sequence region, and the FILL16 sequence region. In one embodiment, the filler sequence region is the FILL5 sequence region, the FILL8 sequence region, and the FILL17 sequence region. In one embodiment, the filler sequence region is the FILL5 sequence region, the FILL8 sequence region, and the FILL18 sequence region. In one embodiment, the filler sequence region is the FILL5 sequence region, the FILL9 sequence region, and the FILL10 sequence region. In one embodiment, the filler sequence region is the FILL5 sequence region, the FILL9 sequence region, and the FILL11 sequence region. In one embodiment, the filler sequence region is the FILL5 sequence region, the FILL9 sequence region, and the FILL12 sequence region. In one embodiment, the filler sequence region is the FILL5 sequence region, the FILL9 sequence region, and the FILL13 sequence region. In one embodiment, the filler sequence region is the FILL5 sequence region, the FILL9 sequence region, and the FILL14 sequence region. In one embodiment, the filler sequence region is the FILL5 sequence region, the FILL9 sequence region, and the FILL15 sequence region. In one embodiment, the filler sequence region is the FILL5 sequence region, the FILL9 sequence region, and the FILL16 sequence region. In one embodiment, the filler sequence region is the FILL5 sequence region, the FILL9 sequence region, and the FILL17 sequence region. In one embodiment, the filler sequence region is the FILL5 sequence region, the FILL9 sequence region, and the FILL18 sequence region. In one embodiment, the filler sequence region is the FILL5 sequence region, the FILL10 sequence region, and the FILL11 sequence region. In one embodiment, the filler sequence region is the FILL5 sequence region, the FILL10 sequence region, and the FILL12 sequence region. In one embodiment, the filler sequence region is the FILL5 sequence region, the FILL10 sequence region, and the FILL13 sequence region. In one embodiment, the filler sequence region is the FILL5 sequence region, the FILL10 sequence region, and the FILL14 sequence region. In one embodiment, the filler sequence region is the FILL5 sequence region, the FILL10 sequence region, and the FILL15 sequence region. In one embodiment, the filler sequence region is the FILL5 sequence region, the FILL10 sequence region, and the FILL16 sequence region. In one embodiment, the filler sequence region is the FILL5 sequence region, the FILL10 sequence region, and the FILL17 sequence region. In one embodiment, the filler sequence region is the FILL5 sequence region, the FILL10 sequence region, and the FILL18 sequence region. In one embodiment, the filler sequence region is the FILL5 sequence region, the FILL11 sequence region, and the FILL12 sequence region. In one embodiment, the filler sequence region is the FILL5 sequence region, the FILL11 sequence region, and the FILL13 sequence region. In one embodiment, the filler sequence region is the FILL5 sequence region, the FILL11 sequence region, and the FILL14 sequence region. In one embodiment, the filler sequence region is the FILL5 sequence region, the FILL11 sequence region, and the FILL15 sequence region. In one embodiment, the filler sequence region is the FILL5 sequence region, the FILL11 sequence region, and the FILL16 sequence region. In one embodiment, the filler sequence region is the FILL5 sequence region, the FILL11 sequence region, and the FILL17 sequence region. In one embodiment, the filler sequence region is the FILL5 sequence region, the FILL11 sequence region, and the FILL18 sequence region. In one embodiment, the filler sequence region is the FILL5 sequence region, the FILL12 sequence region, and the FILL13 sequence region. In one embodiment, the filler sequence region is the FILL5 sequence region, the FILL12 sequence region, and the FILL14 sequence region. In one embodiment, the filler sequence region is the FILL5 sequence region, the FILL12 sequence region, and the FILL15 sequence region. In one embodiment, the filler sequence region is the FILL5 sequence region, the FILL12 sequence region, and the FILL16 sequence region. In one embodiment, the filler sequence region is the FILL5 sequence region, the FILL12 sequence region, and the FILL17 sequence region. In one embodiment, the filler sequence region is the FILL5 sequence region, the FILL12 sequence region, and the FILL18 sequence region. In one embodiment, the filler sequence region is the FILL5 sequence region, the FILL13 sequence region, and the FILL14 sequence region. In one embodiment, the filler sequence region is the FILL5 sequence region, the FILL13 sequence region, and the FILL15 sequence region. In one embodiment, the filler sequence region is the FILL5 sequence region, the FILL13 sequence region, and the FILL16 sequence region. In one embodiment, the filler sequence region is the FILL5 sequence region, the FILL13 sequence region, and the FILL17 sequence region. In one embodiment, the filler sequence region is the FILL5 sequence region, the FILL13 sequence region, and the FILL18 sequence region. In one embodiment, the filler sequence region is the FILL5 sequence region, the FILL14 sequence region, and the FILL15 sequence region. In one embodiment, the filler sequence region is the FILL5 sequence region, the FILL14 sequence region, and the FILL16 sequence region. In one embodiment, the filler sequence region is the FILL5 sequence region, the FILL14 sequence region, and the FILL17 sequence region. In one embodiment, the filler sequence region is the FILL5 sequence region, the FILL14 sequence region, and the FILL18 sequence region. In one embodiment, the filler sequence region is the FILL5 sequence region, the FILL15 sequence region, and the FILL16 sequence region. In one embodiment, the filler sequence region is the FILL5 sequence region, the FILL15 sequence region, and the FILL17 sequence region. In one embodiment, the filler sequence region is the FILL5 sequence region, the FILL15 sequence region, and the FILL18 sequence region. In one embodiment, the filler sequence region is the FILL5 sequence region, the FILL16 sequence region, and the FILL17 sequence region. In one embodiment, the filler sequence region is the FILL5 sequence region, the FILL16 sequence region, and the FILL18 sequence region. In one embodiment, the filler sequence region is the FILL5 sequence region, the FILL17 sequence region, and the FILL18 sequence region. In one embodiment, the filler sequence region is the FILL6 sequence region, the FILL7 sequence region, and the FILL8 sequence region. In one embodiment, the filler sequence region is the FILL6 sequence region, the FILL7 sequence region, and the FILL9 sequence region. In one embodiment, the filler sequence region is the FILL6 sequence region, the FILL7 sequence region, and the FILL10 sequence region. In one embodiment, the filler sequence region is the FILL6 sequence region, the FILL7 sequence region, and the FILL11 sequence region. In one embodiment, the filler sequence region is the FILL6 sequence region, the FILL7 sequence region, and the FILL12 sequence region. In one embodiment, the filler sequence region is the FILL6 sequence region, the FILL7 sequence region, and the FILL13 sequence region. In one embodiment, the filler sequence region is the FILL6 sequence region, the FILL7 sequence region, and the FILL14 sequence region. In one embodiment, the filler sequence region is the FILL6 sequence region, the FILL7 sequence region, and the FILL15 sequence region. In one embodiment, the filler sequence region is the FILL6 sequence region, the FILL7 sequence region, and the FILL16 sequence region. In one embodiment, the filler sequence region is the FILL6 sequence region, the FILL7 sequence region, and the FILL17 sequence region. In one embodiment, the filler sequence region is the FILL6 sequence region, the FILL7 sequence region, and the FILL18 sequence region. In one embodiment, the filler sequence region is the FILL6 sequence region, the FILL8 sequence region, and the FILL9 sequence region. In one embodiment, the filler sequence region is the FILL6 sequence region, the FILL8 sequence region, and the FILL10 sequence region. In one embodiment, the filler sequence region is the FILL6 sequence region, the FILL8 sequence region, and the FILL11 sequence region. In one embodiment, the filler sequence region is the FILL6 sequence region, the FILL8 sequence region, and the FILL12 sequence region. In one embodiment, the filler sequence region is the FILL6 sequence region, the FILL8 sequence region, and the FILL13 sequence region. In one embodiment, the filler sequence region is the FILL6 sequence region, the FILL8 sequence region, and the FILL14 sequence region. In one embodiment, the filler sequence region is the FILL6 sequence region, the FILL8 sequence region, and the FILL15 sequence region. In one embodiment, the filler sequence region is the FILL6 sequence region, the FILL8 sequence region, and the FILL16 sequence region. In one embodiment, the filler sequence region is the FILL6 sequence region, the FILL8 sequence region, and the FILL17 sequence region. In one embodiment, the filler sequence region is the FILL6 sequence region, the FILL8 sequence region, and the FILL18 sequence region. In one embodiment, the filler sequence region is the FILL6 sequence region, the FILL9 sequence region, and the FILL10 sequence region. In one embodiment, the filler sequence region is the FILL6 sequence region, the FILL9 sequence region, and the FILL11 sequence region. In one embodiment, the filler sequence region is the FILL6 sequence region, the FILL9 sequence region, and the FILL12 sequence region. In one embodiment, the filler sequence region is the FILL6 sequence region, the FILL9 sequence region, and the FILL13 sequence region. In one embodiment, the filler sequence region is the FILL6 sequence region, the FILL9 sequence region, and the FILL14 sequence region. In one embodiment, the filler sequence region is the FILL6 sequence region, the FILL9 sequence region, and the FILL15 sequence region. In one embodiment, the filler sequence region is the FILL6 sequence region, the FILL9 sequence region, and the FILL16 sequence region. In one embodiment, the filler sequence region is the FILL6 sequence region, the FILL9 sequence region, and the FILL17 sequence region. In one embodiment, the filler sequence region is the FILL6 sequence region, the FILL9 sequence region, and the FILL18 sequence region. In one embodiment, the filler sequence region is the FILL6 sequence region, the FILL10 sequence region, and the FILL11 sequence region. In one embodiment, the filler sequence region is the FILL6 sequence region, the FILL10 sequence region, and the FILL12 sequence region. In one embodiment, the filler sequence region is the FILL6 sequence region, the FILL10 sequence region, and the FILL13 sequence region. In one embodiment, the filler sequence region is the FILL6 sequence region, the FILL10 sequence region, and the FILL14 sequence region. In one embodiment, the filler sequence region is the FILL6 sequence region, the FILL10 sequence region, and the FILL15 sequence region. In one embodiment, the filler sequence region is the FILL6 sequence region, the FILL10 sequence region, and the FILL16 sequence region. In one embodiment, the filler sequence region is the FILL6 sequence region, the FILL10 sequence region, and the FILL17 sequence region. In one embodiment, the filler sequence region is the FILL6 sequence region, the FILL10 sequence region, and the FILL18 sequence region. In one embodiment, the filler sequence region is the FILL6 sequence region, the FILL11 sequence region, and the FILL12 sequence region. In one embodiment, the filler sequence region is the FILL6 sequence region, the FILL11 sequence region, and the FILL13 sequence region. In one embodiment, the filler sequence region is the FILL6 sequence region, the FILL11 sequence region, and the FILL14 sequence region. In one embodiment, the filler sequence region is the FILL6 sequence region, the FILL11 sequence region, and the FILL15 sequence region. In one embodiment, the filler sequence region is the FILL6 sequence region, the FILL11 sequence region, and the FILL16 sequence region. In one embodiment, the filler sequence region is the FILL6 sequence region, the FILL11 sequence region, and the FILL17 sequence region. In one embodiment, the filler sequence region is the FILL6 sequence region, the FILL11 sequence region, and the FILL18 sequence region. In one embodiment, the filler sequence region is the FILL6 sequence region, the FILL12 sequence region, and the FILL13 sequence region. In one embodiment, the filler sequence region is the FILL6 sequence region, the FILL12 sequence region, and the FILL14 sequence region. In one embodiment, the filler sequence region is the FILL6 sequence region, the FILL12 sequence region, and the FILL15 sequence region. In one embodiment, the filler sequence region is the FILL6 sequence region, the FILL12 sequence region, and the FILL16 sequence region. In one embodiment, the filler sequence region is the FILL6 sequence region, the FILL12 sequence region, and the FILL17 sequence region. In one embodiment, the filler sequence region is the FILL6 sequence region, the FILL12 sequence region, and the FILL18 sequence region. In one embodiment, the filler sequence region is the FILL6 sequence region, the FILL13 sequence region, and the FILL14 sequence region. In one embodiment, the filler sequence region is the FILL6 sequence region, the FILL13 sequence region, and the FILL15 sequence region. In one embodiment, the filler sequence region is the FILL6 sequence region, the FILL13 sequence region, and the FILL16 sequence region. In one embodiment, the filler sequence region is the FILL6 sequence region, the FILL13 sequence region, and the FILL17 sequence region. In one embodiment, the filler sequence region is the FILL6 sequence region, the FILL13 sequence region, and the FILL18 sequence region. In one embodiment, the filler sequence region is the FILL6 sequence region, the FILL14 sequence region, and the FILL15 sequence region. In one embodiment, the filler sequence region is the FILL6 sequence region, the FILL14 sequence region, and the FILL16 sequence region. In one embodiment, the filler sequence region is the FILL6 sequence region, the FILL14 sequence region, and the FILL17 sequence region. In one embodiment, the filler sequence region is the FILL6 sequence region, the FILL14 sequence region, and the FILL18 sequence region. In one embodiment, the filler sequence region is the FILL6 sequence region, the FILL15 sequence region, and the FILL16 sequence region. In one embodiment, the filler sequence region is the FILL6 sequence region, the FILL15 sequence region, and the FILL17 sequence region. In one embodiment, the filler sequence region is the FILL6 sequence region, the FILL15 sequence region, and the FILL18 sequence region. In one embodiment, the filler sequence region is the FILL6 sequence region, the FILL16 sequence region, and the FILL17 sequence region. In one embodiment, the filler sequence region is the FILL6 sequence region, the FILL16 sequence region, and the FILL18 sequence region. In one embodiment, the filler sequence region is the FILL6 sequence region, the FILL17 sequence region, and the FILL18 sequence region. In one embodiment, the filler sequence region is the FILL7 sequence region, the FILL8 sequence region, and the FILL9 sequence region. In one embodiment, the filler sequence region is the FILL7 sequence region, the FILL8 sequence region, and the FILL10 sequence region. In one embodiment, the filler sequence region is the FILL7 sequence region, the FILL8 sequence region, and the FILL11 sequence region. In one embodiment, the filler sequence region is the FILL7 sequence region, the FILL8 sequence region, and the FILL12 sequence region. In one embodiment, the filler sequence region is the FILL7 sequence region, the FILL8 sequence region, and the FILL13 sequence region. In one embodiment, the filler sequence region is the FILL7 sequence region, the FILL8 sequence region, and the FILL14 sequence region. In one embodiment, the filler sequence region is the FILL7 sequence region, the FILL8 sequence region, and the FILL15 sequence region. In one embodiment, the filler sequence region is the FILL7 sequence region, the FILL8 sequence region, and the FILL16 sequence region. In one embodiment, the filler sequence region is the FILL7 sequence region, the FILL8 sequence region, and the FILL17 sequence region. In one embodiment, the filler sequence region is the FILL7 sequence region, the FILL8 sequence region, and the FILL18 sequence region. In one embodiment, the filler sequence region is the FILL7 sequence region, the FILL9 sequence region, and the FILL10 sequence region. In one embodiment, the filler sequence region is the FILL7 sequence region, the FILL9 sequence region, and the FILL11 sequence region. In one embodiment, the filler sequence region is the FILL7 sequence region, the FILL9 sequence region, and the FILL12 sequence region. In one embodiment, the filler sequence region is the FILL7 sequence region, the FILL9 sequence region, and the FILL13 sequence region. In one embodiment, the filler sequence region is the FILL7 sequence region, the FILL9 sequence region, and the FILL14 sequence region. In one embodiment, the filler sequence region is the FILL7 sequence region, the FILL9 sequence region, and the FILL15 sequence region. In one embodiment, the filler sequence region is the FILL7 sequence region, the FILL9 sequence region, and the FILL16 sequence region. In one embodiment, the filler sequence region is the FILL7 sequence region, the FILL9 sequence region, and the FILL17 sequence region. In one embodiment, the filler sequence region is the FILL7 sequence region, the FILL9 sequence region, and the FILL18 sequence region. In one embodiment, the filler sequence region is the FILL7 sequence region, the FILL10 sequence region, and the FILL11 sequence region. In one embodiment, the filler sequence region is the FILL7 sequence region, the FILL10 sequence region, and the FILL12 sequence region. In one embodiment, the filler sequence region is the FILL7 sequence region, the FILL10 sequence region, and the FILL13 sequence region. In one embodiment, the filler sequence region is the FILL7 sequence region, the FILL10 sequence region, and the FILL14 sequence region. In one embodiment, the filler sequence region is the FILL7 sequence region, the FILL10 sequence region, and the FILL15 sequence region. In one embodiment, the filler sequence region is the FILL7 sequence region, the FILL10 sequence region, and the FILL16 sequence region. In one embodiment, the filler sequence region is the FILL7 sequence region, the FILL10 sequence region, and the FILL17 sequence region. In one embodiment, the filler sequence region is the FILL7 sequence region, the FILL10 sequence region, and the FILL18 sequence region. In one embodiment, the filler sequence region is the FILL7 sequence region, the FILL11 sequence region, and the FILL12 sequence region. In one embodiment, the filler sequence region is the FILL7 sequence region, the FILL11 sequence region, and the FILL13 sequence region. In one embodiment, the filler sequence region is the FILL7 sequence region, the FILL11 sequence region, and the FILL14 sequence region. In one embodiment, the filler sequence region is the FILL7 sequence region, the FILL11 sequence region, and the FILL15 sequence region. In one embodiment, the filler sequence region is the FILL7 sequence region, the FILL11 sequence region, and the FILL16 sequence region. In one embodiment, the filler sequence region is the FILL7 sequence region, the FILL11 sequence region, and the FILL17 sequence region. In one embodiment, the filler sequence region is the FILL7 sequence region, the FILL11 sequence region, and the FILL18 sequence region. In one embodiment, the filler sequence region is the FILL7 sequence region, the FILL12 sequence region, and the FILL13 sequence region. In one embodiment, the filler sequence region is the FILL7 sequence region, the FILL12 sequence region, and the FILL14 sequence region. In one embodiment, the filler sequence region is the FILL7 sequence region, the FILL12 sequence region, and the FILL15 sequence region. In one embodiment, the filler sequence region is the FILL7 sequence region, the FILL12 sequence region, and the FILL16 sequence region. In one embodiment, the filler sequence region is the FILL7 sequence region, the FILL12 sequence region, and the FILL17 sequence region. In one embodiment, the filler sequence region is the FILL7 sequence region, the FILL12 sequence region, and the FILL18 sequence region. In one embodiment, the filler sequence region is the FILL7 sequence region, the FILL13 sequence region, and the FILL14 sequence region. In one embodiment, the filler sequence region is the FILL7 sequence region, the FILL13 sequence region, and the FILL15 sequence region. In one embodiment, the filler sequence region is the FILL7 sequence region, the FILL13 sequence region, and the FILL16 sequence region. In one embodiment, the filler sequence region is the FILL7 sequence region, the FILL13 sequence region, and the FILL17 sequence region. In one embodiment, the filler sequence region is the FILL7 sequence region, the FILL13 sequence region, and the FILL18 sequence region. In one embodiment, the filler sequence region is the FILL7 sequence region, the FILL14 sequence region, and the FILL15 sequence region. In one embodiment, the filler sequence region is the FILL7 sequence region, the FILL14 sequence region, and the FILL16 sequence region. In one embodiment, the filler sequence region is the FILL7 sequence region, the FILL14 sequence region, and the FILL17 sequence region. In one embodiment, the filler sequence region is the FILL7 sequence region, the FILL14 sequence region, and the FILL18 sequence region. In one embodiment, the filler sequence region is the FILL7 sequence region, the FILL15 sequence region, and the FILL16 sequence region. In one embodiment, the filler sequence region is the FILL7 sequence region, the FILL15 sequence region, and the FILL17 sequence region. In one embodiment, the filler sequence region is the FILL7 sequence region, the FILL15 sequence region, and the FILL18 sequence region. In one embodiment, the filler sequence region is the FILL7 sequence region, the FILL16 sequence region, and the FILL17 sequence region. In one embodiment, the filler sequence region is the FILL7 sequence region, the FILL16 sequence region, and the FILL18 sequence region. In one embodiment, the filler sequence region is the FILL7 sequence region, the FILL17 sequence region, and the FILL18 sequence region. In one embodiment, the filler sequence region is the FILL8 sequence region, the FILL9 sequence region, and the FILL10 sequence region. In one embodiment, the filler sequence region is the FILL8 sequence region, the FILL9 sequence region, and the FILL11 sequence region. In one embodiment, the filler sequence region is the FILL8 sequence region, the FILL9 sequence region, and the FILL12 sequence region. In one embodiment, the filler sequence region is the FILL8 sequence region, the FILL9 sequence region, and the FILL13 sequence region. In one embodiment, the filler sequence region is the FILL8 sequence region, the FILL9 sequence region, and the FILL14 sequence region. In one embodiment, the filler sequence region is the FILL8 sequence region, the FILL9 sequence region, and the FILL15 sequence region. In one embodiment, the filler sequence region is the FILL8 sequence region, the FILL9 sequence region, and the FILL16 sequence region. In one embodiment, the filler sequence region is the FILL8 sequence region, the FILL9 sequence region, and the FILL17 sequence region. In one embodiment, the filler sequence region is the FILL8 sequence region, the FILL9 sequence region, and the FILL18 sequence region. In one embodiment, the filler sequence region is the FILL8 sequence region, the FILL10 sequence region, and the FILL11 sequence region. In one embodiment, the filler sequence region is the FILL8 sequence region, the FILL10 sequence region, and the FILL12 sequence region. In one embodiment, the filler sequence region is the FILL8 sequence region, the FILL10 sequence region, and the FILL13 sequence region. In one embodiment, the filler sequence region is the FILL8 sequence region, the FILL10 sequence region, and the FILL14 sequence region. In one embodiment, the filler sequence region is the FILL8 sequence region, the FILL10 sequence region, and the FILL15 sequence region. In one embodiment, the filler sequence region is the FILL8 sequence region, the FILL10 sequence region, and the FILL16 sequence region. In one embodiment, the filler sequence region is the FILL8 sequence region, the FILL10 sequence region, and the FILL17 sequence region. In one embodiment, the filler sequence region is the FILL8 sequence region, the FILL10 sequence region, and the FILL18 sequence region. In one embodiment, the filler sequence region is the FILL8 sequence region, the FILL11 sequence region, and the FILL12 sequence region. In one embodiment, the filler sequence region is the FILL8 sequence region, the FILL11 sequence region, and the FILL13 sequence region. In one embodiment, the filler sequence region is the FILL8 sequence region, the FILL11 sequence region, and the FILL14 sequence region. In one embodiment, the filler sequence region is the FILL8 sequence region, the FILL11 sequence region, and the FILL15 sequence region. In one embodiment, the filler sequence region is the FILL8 sequence region, the FILL11 sequence region, and the FILL16 sequence region. In one embodiment, the filler sequence region is the FILL8 sequence region, the FILL11 sequence region, and the FILL17 sequence region. In one embodiment, the filler sequence region is the FILL8 sequence region, the FILL11 sequence region, and the FILL18 sequence region. In one embodiment, the filler sequence region is the FILL8 sequence region, the FILL12 sequence region, and the FILL13 sequence region. In one embodiment, the filler sequence region is the FILL8 sequence region, the FILL12 sequence region, and the FILL14 sequence region. In one embodiment, the filler sequence region is the FILL8 sequence region, the FILL12 sequence region, and the FILL15 sequence region. In one embodiment, the filler sequence region is the FILL8 sequence region, the FILL12 sequence region, and the FILL16 sequence region. In one embodiment, the filler sequence region is the FILL8 sequence region, the FILL12 sequence region, and the FILL17 sequence region. In one embodiment, the filler sequence region is the FILL8 sequence region, the FILL12 sequence region, and the FILL18 sequence region. In one embodiment, the filler sequence region is the FILL8 sequence region, the FILL13 sequence region, and the FILL14 sequence region. In one embodiment, the filler sequence region is the FILL8 sequence region, the FILL13 sequence region, and the FILL15 sequence region. In one embodiment, the filler sequence region is the FILL8 sequence region, the FILL13 sequence region, and the FILL16 sequence region. In one embodiment, the filler sequence region is the FILL8 sequence region, the FILL13 sequence region, and the FILL17 sequence region. In one embodiment, the filler sequence region is the FILL8 sequence region, the FILL13 sequence region, and the FILL18 sequence region. In one embodiment, the filler sequence region is the FILL8 sequence region, the FILL14 sequence region, and the FILL15 sequence region. In one embodiment, the filler sequence region is the FILL8 sequence region, the FILL14 sequence region, and the FILL16 sequence region. In one embodiment, the filler sequence region is the FILL8 sequence region, the FILL14 sequence region, and the FILL17 sequence region. In one embodiment, the filler sequence region is the FILL8 sequence region, the FILL14 sequence region, and the FILL18 sequence region. In one embodiment, the filler sequence region is the FILL8 sequence region, the FILL15 sequence region, and the FILL16 sequence region. In one embodiment, the filler sequence region is the FILL8 sequence region, the FILL15 sequence region, and the FILL17 sequence region. In one embodiment, the filler sequence region is the FILL8 sequence region, the FILL15 sequence region, and the FILL18 sequence region. In one embodiment, the filler sequence region is the FILL8 sequence region, the FILL16 sequence region, and the FILL17 sequence region. In one embodiment, the filler sequence region is the FILL8 sequence region, the FILL16 sequence region, and the FILL18 sequence region. In one embodiment, the filler sequence region is the FILL8 sequence region, the FILL17 sequence region, and the FILL18 sequence region. In one embodiment, the filler sequence region is the FILL9 sequence region, the FILL10 sequence region, and the FILL11 sequence region. In one embodiment, the filler sequence region is the FILL9 sequence region, the FILL10 sequence region, and the FILL12 sequence region. In one embodiment, the filler sequence region is the FILL9 sequence region, the FILL10 sequence region, and the FILL13 sequence region. In one embodiment, the filler sequence region is the FILL9 sequence region, the FILL10 sequence region, and the FILL14 sequence region. In one embodiment, the filler sequence region is the FILL9 sequence region, the FILL10 sequence region, and the FILL15 sequence region. In one embodiment, the filler sequence region is the FILL9 sequence region, the FILL10 sequence region, and the FILL16 sequence region. In one embodiment, the filler sequence region is the FILL9 sequence region, the FILL10 sequence region, and the FILL17 sequence region. In one embodiment, the filler sequence region is the FILL9 sequence region, the FILL10 sequence region, and the FILL18 sequence region. In one embodiment, the filler sequence region is the FILL9 sequence region, the FILL11 sequence region, and the FILL12 sequence region. In one embodiment, the filler sequence region is the FILL9 sequence region, the FILL11 sequence region, and the FILL13 sequence region. In one embodiment, the filler sequence region is the FILL9 sequence region, the FILL11 sequence region, and the FILL14 sequence region. In one embodiment, the filler sequence region is the FILL9 sequence region, the FILL11 sequence region, and the FILL15 sequence region. In one embodiment, the filler sequence region is the FILL9 sequence region, the FILL11 sequence region, and the FILL16 sequence region. In one embodiment, the filler sequence region is the FILL9 sequence region, the FILL11 sequence region, and the FILL17 sequence region. In one embodiment, the filler sequence region is the FILL9 sequence region, the FILL11 sequence region, and the FILL18 sequence region. In one embodiment, the filler sequence region is the FILL9 sequence region, the FILL12 sequence region, and the FILL13 sequence region. In one embodiment, the filler sequence region is the FILL9 sequence region, the FILL12 sequence region, and the FILL14 sequence region. In one embodiment, the filler sequence region is the FILL9 sequence region, the FILL12 sequence region, and the FILL15 sequence region. In one embodiment, the filler sequence region is the FILL9 sequence region, the FILL12 sequence region, and the FILL16 sequence region. In one embodiment, the filler sequence region is the FILL9 sequence region, the FILL12 sequence region, and the FILL17 sequence region. In one embodiment, the filler sequence region is the FILL9 sequence region, the FILL12 sequence region, and the FILL18 sequence region. In one embodiment, the filler sequence region is the FILL9 sequence region, the FILL13 sequence region, and the FILL14 sequence region. In one embodiment, the filler sequence region is the FILL9 sequence region, the FILL13 sequence region, and the FILL15 sequence region. In one embodiment, the filler sequence region is the FILL9 sequence region, the FILL13 sequence region, and the FILL16 sequence region. In one embodiment, the filler sequence region is the FILL9 sequence region, the FILL13 sequence region, and the FILL17 sequence region. In one embodiment, the filler sequence region is the FILL9 sequence region, the FILL13 sequence region, and the FILL18 sequence region. In one embodiment, the filler sequence region is the FILL9 sequence region, the FILL14 sequence region, and the FILL15 sequence region. In one embodiment, the filler sequence region is the FILL9 sequence region, the FILL14 sequence region, and the FILL16 sequence region. In one embodiment, the filler sequence region is the FILL9 sequence region, the FILL14 sequence region, and the FILL17 sequence region. In one embodiment, the filler sequence region is the FILL9 sequence region, the FILL14 sequence region, and the FILL18 sequence region. In one embodiment, the filler sequence region is the FILL9 sequence region, the FILL15 sequence region, and the FILL16 sequence region. In one embodiment, the filler sequence region is the FILL9 sequence region, the FILL15 sequence region, and the FILL17 sequence region. In one embodiment, the filler sequence region is the FILL9 sequence region, the FILL15 sequence region, and the FILL18 sequence region. In one embodiment, the filler sequence region is the FILL9 sequence region, the FILL16 sequence region, and the FILL17 sequence region. In one embodiment, the filler sequence region is the FILL9 sequence region, the FILL16 sequence region, and the FILL18 sequence region. In one embodiment, the filler sequence region is the FILL9 sequence region, the FILL17 sequence region, and the FILL18 sequence region. In one embodiment, the filler sequence region is the FILL10 sequence region, the FILL11 sequence region, and the FILL12 sequence region. In one embodiment, the filler sequence region is the FILL10 sequence region, the FILL11 sequence region, and the FILL13 sequence region. In one embodiment, the filler sequence region is the FILL10 sequence region, the FILL11 sequence region, and the FILL14 sequence region. In one embodiment, the filler sequence region is the FILL10 sequence region, the FILL11 sequence region, and the FILL15 sequence region. In one embodiment, the filler sequence region is the FILL10 sequence region, the FILL11 sequence region, and the FILL16 sequence region. In one embodiment, the filler sequence region is the FILL10 sequence region, the FILL11 sequence region, and the FILL17 sequence region. In one embodiment, the filler sequence region is the FILL10 sequence region, the FILL11 sequence region, and the FILL18 sequence region. In one embodiment, the filler sequence region is the FILL10 sequence region, the FILL12 sequence region, and the FILL13 sequence region. In one embodiment, the filler sequence region is the FILL10 sequence region, the FILL12 sequence region, and the FILL14 sequence region. In one embodiment, the filler sequence region is the FILL10 sequence region, the FILL12 sequence region, and the FILL15 sequence region. In one embodiment, the filler sequence region is the FILL10 sequence region, the FILL12 sequence region, and the FILL16 sequence region. In one embodiment, the filler sequence region is the FILL10 sequence region, the FILL12 sequence region, and the FILL17 sequence region. In one embodiment, the filler sequence region is the FILL10 sequence region, the FILL12 sequence region, and the FILL18 sequence region. In one embodiment, the filler sequence region is the FILL10 sequence region, the FILL13 sequence region, and the FILL14 sequence region. In one embodiment, the filler sequence region is the FILL10 sequence region, the FILL13 sequence region, and the FILL15 sequence region. In one embodiment, the filler sequence region is the FILL10 sequence region, the FILL13 sequence region, and the FILL16 sequence region. In one embodiment, the filler sequence region is the FILL10 sequence region, the FILL13 sequence region, and the FILL17 sequence region. In one embodiment, the filler sequence region is the FILL10 sequence region, the FILL13 sequence region, and the FILL18 sequence region. In one embodiment, the filler sequence region is the FILL10 sequence region, the FILL14 sequence region, and the FILL15 sequence region. In one embodiment, the filler sequence region is the FILL10 sequence region, the FILL14 sequence region, and the FILL16 sequence region. In one embodiment, the filler sequence region is the FILL10 sequence region, the FILL14 sequence region, and the FILL17 sequence region. In one embodiment, the filler sequence region is the FILL10 sequence region, the FILL14 sequence region, and the FILL18 sequence region. In one embodiment, the filler sequence region is the FILL10 sequence region, the FILL15 sequence region, and the FILL16 sequence region. In one embodiment, the filler sequence region is the FILL10 sequence region, the FILL15 sequence region, and the FILL17 sequence region. In one embodiment, the filler sequence region is the FILL10 sequence region, the FILL15 sequence region, and the FILL18 sequence region. In one embodiment, the filler sequence region is the FILL10 sequence region, the FILL16 sequence region, and the FILL17 sequence region. In one embodiment, the filler sequence region is the FILL10 sequence region, the FILL16 sequence region, and the FILL18 sequence region. In one embodiment, the filler sequence region is the FILL10 sequence region, the FILL17 sequence region, and the FILL18 sequence region. In one embodiment, the filler sequence region is the FILL11 sequence region, the FILL12 sequence region, and the FILL13 sequence region. In one embodiment, the filler sequence region is the FILL11 sequence region, the FILL12 sequence region, and the FILL14 sequence region. In one embodiment, the filler sequence region is the FILL11 sequence region, the FILL12 sequence region, and the FILL15 sequence region. In one embodiment, the filler sequence region is the FILL11 sequence region, the FILL12 sequence region, and the FILL16 sequence region. In one embodiment, the filler sequence region is the FILL11 sequence region, the FILL12 sequence region, and the FILL17 sequence region. In one embodiment, the filler sequence region is the FILL11 sequence region, the FILL12 sequence region, and the FILL18 sequence region. In one embodiment, the filler sequence region is the FILL11 sequence region, the FILL13 sequence region, and the FILL14 sequence region. In one embodiment, the filler sequence region is the FILL11 sequence region, the FILL13 sequence region, and the FILL15 sequence region. In one embodiment, the filler sequence region is the FILL11 sequence region, the FILL13 sequence region, and the FILL16 sequence region. In one embodiment, the filler sequence region is the FILL11 sequence region, the FILL13 sequence region, and the FILL17 sequence region. In one embodiment, the filler sequence region is the FILL11 sequence region, the FILL13 sequence region, and the FILL18 sequence region. In one embodiment, the filler sequence region is the FILL11 sequence region, the FILL14 sequence region, and the FILL15 sequence region. In one embodiment, the filler sequence region is the FILL11 sequence region, the FILL14 sequence region, and the FILL16 sequence region. In one embodiment, the filler sequence region is the FILL11 sequence region, the FILL14 sequence region, and the FILL17 sequence region. In one embodiment, the filler sequence region is the FILL11 sequence region, the FILL14 sequence region, and the FILL18 sequence region. In one embodiment, the filler sequence region is the FILL11 sequence region, the FILL15 sequence region, and the FILL16 sequence region. In one embodiment, the filler sequence region is the FILL11 sequence region, the FILL15 sequence region, and the FILL17 sequence region. In one embodiment, the filler sequence region is the FILL11 sequence region, the FILL15 sequence region, and the FILL18 sequence region. In one embodiment, the filler sequence region is the FILL11 sequence region, the FILL16 sequence region, and the FILL17 sequence region. In one embodiment, the filler sequence region is the FILL11 sequence region, the FILL16 sequence region, and the FILL18 sequence region. In one embodiment, the filler sequence region is the FILL11 sequence region, the FILL17 sequence region, and the FILL18 sequence region. In one embodiment, the filler sequence region is the FILL12 sequence region, the FILL13 sequence region, and the FILL14 sequence region. In one embodiment, the filler sequence region is the FILL12 sequence region, the FILL13 sequence region, and the FILL15 sequence region. In one embodiment, the filler sequence region is the FILL12 sequence region, the FILL13 sequence region, and the FILL16 sequence region. In one embodiment, the filler sequence region is the FILL12 sequence region, the FILL13 sequence region, and the FILL17 sequence region. In one embodiment, the filler sequence region is the FILL12 sequence region, the FILL13 sequence region, and the FILL18 sequence region. In one embodiment, the filler sequence region is the FILL12 sequence region, the FILL14 sequence region, and the FILL15 sequence region. In one embodiment, the filler sequence region is the FILL12 sequence region, the FILL14 sequence region, and the FILL16 sequence region. In one embodiment, the filler sequence region is the FILL12 sequence region, the FILL14 sequence region, and the FILL17 sequence region. In one embodiment, the filler sequence region is the FILL12 sequence region, the FILL14 sequence region, and the FILL18 sequence region. In one embodiment, the filler sequence region is the FILL12 sequence region, the FILL15 sequence region, and the FILL16 sequence region. In one embodiment, the filler sequence region is the FILL12 sequence region, the FILL15 sequence region, and the FILL17 sequence region. In one embodiment, the filler sequence region is the FILL12 sequence region, the FILL15 sequence region, and the FILL18 sequence region. In one embodiment, the filler sequence region is the FILL12 sequence region, the FILL16 sequence region, and the FILL17 sequence region. In one embodiment, the filler sequence region is the FILL12 sequence region, the FILL16 sequence region, and the FILL18 sequence region. In one embodiment, the filler sequence region is the FILL12 sequence region, the FILL17 sequence region, and the FILL18 sequence region. In one embodiment, the filler sequence region is the FILL13 sequence region, the FILL14 sequence region, and the FILL15 sequence region. In one embodiment, the filler sequence region is the FILL13 sequence region, the FILL14 sequence region, and the FILL16 sequence region. In one embodiment, the filler sequence region is the FILL13 sequence region, the FILL14 sequence region, and the FILL17 sequence region. In one embodiment, the filler sequence region is the FILL13 sequence region, the FILL14 sequence region, and the FILL18 sequence region. In one embodiment, the filler sequence region is the FILL13 sequence region, the FILL15 sequence region, and the FILL16 sequence region. In one embodiment, the filler sequence region is the FILL13 sequence region, the FILL15 sequence region, and the FILL17 sequence region. In one embodiment, the filler sequence region is the FILL13 sequence region, the FILL15 sequence region, and the FILL18 sequence region. In one embodiment, the filler sequence region is the FILL13 sequence region, the FILL16 sequence region, and the FILL17 sequence region. In one embodiment, the filler sequence region is the FILL13 sequence region, the FILL16 sequence region, and the FILL18 sequence region. In one embodiment, the filler sequence region is the FILL13 sequence region, the FILL17 sequence region, and the FILL18 sequence region. In one embodiment, the filler sequence region is the FILL14 sequence region, the FILL15 sequence region, and the FILL16 sequence region. In one embodiment, the filler sequence region is the FILL14 sequence region, the FILL15 sequence region, and the FILL17 sequence region. In one embodiment, the filler sequence region is the FILL14 sequence region, the FILL15 sequence region, and the FILL18 sequence region. In one embodiment, the filler sequence region is the FILL14 sequence region, the FILL16 sequence region, and the FILL17 sequence region. In one embodiment, the filler sequence region is the FILL14 sequence region, the FILL16 sequence region, and the FILL18 sequence region. In one embodiment, the filler sequence region is the FILL14 sequence region, the FILL17 sequence region, and the FILL18 sequence region. In one embodiment, the filler sequence region is the FILL15 sequence region, the FILL16 sequence region, and the FILL17 sequence region. In one embodiment, the filler sequence region is the FILL15 sequence region, the FILL16 sequence region, and the FILL18 sequence region. In one embodiment, the filler sequence region is the FILL15 sequence region, the FILL17 sequence region, and the FILL18 sequence region. In one embodiment, the filler sequence region is the FILL16 sequence region, the FILL17 sequence region, and the FILL18 sequence region.

In one embodiment, the AAV particle viral genome may comprise at least one enhancer sequence region. The enhancer sequence region(s) may, independently, have a length such as, but not limited to, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, and 400 nucleotides. The length of the enhancer region for the viral genome may be 300-310, 300-325, 305-315, 310-320, 315-325, 320-330, 325-335, 325-350, 330-340, 335-345, 340-350, 345-355, 350-360, 350-375, 355-365, 360-370, 365-375, 370-380, 375-385, 375-400, 380-390, 385-395, and 390-400 nucleotides. As a non-limiting example, the viral genome comprises an enhancer region that is about 303 nucleotides in length. As a non-limiting example, the viral genome comprises an enhancer region that is about 382 nucleotides in length.

In one embodiment, the AAV particle viral genome comprises at least one enhancer sequence region. Non-limiting examples of enhancer sequence regions are described in Table 20.

TABLE 20
Enhancer Sequence Regions

| Sequence Region Name | SEQ ID NO |
|---|---|
| Enhancer1 | 1814 |
| Enhancer2 | 1815 |

In one embodiment, the AAV particle viral genome comprises one enhancer sequence region. In one embodiment, the enhancer sequence regions is the Enhancer1 sequence region. In one embodiment, the enhancer sequence regions is the Enhancer2 sequence region.

In one embodiment, the AAV particle viral genome comprises two enhancer sequence regions. In one embodiment, the enhancer sequence regions are the Enhancer1 sequence region and the Enhancer 2 sequence region.

In one embodiment, the AAV particle viral genome may comprise at least one promoter sequence region. The promoter sequence region(s) may, independently, have a length such as, but not limited to, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 nucleotides. The length of the promoter region for the viral genome may be 4-10, 10-20, 10-50, 20-30, 30-40, 40-50, 50-60, 50-100, 60-70, 70-80, 80-90, 90-100, 100-110, 100-150, 110-120, 120-130, 130-140, 140-150, 150-160, 150-200, 160-170, 170-180, 180-190, 190-200, 200-210, 200-250, 210-220, 220-230, 230-240, 240-250, 250-260, 250-300, 260-270, 270-280, 280-290, 290-300, 300-310, 300-350, 310-320, 320-330, 330-340, 340-350, 350-360, 350-400, 360-370, 370-380, 380-390, 390-400, 400-410, 400-450, 410-420, 420-430, 430-440, 440-450, 450-460, 450-500, 460-470, 470-480, 480-490, 490-500, 500-510, 500-550, 510-520, 520-530, 530-540, 540-550, 550-560, 550-600, 560-570, 570-580, 580-590, and 590-600 nucleotides. As a non-limiting example, the viral genome comprises a promoter region that is about 4 nucleotides in length. As a non-limiting example, the viral genome comprises a promoter region that is about 17 nucleotides in length. As a non-limiting example, the viral genome comprises a promoter region that is about 204 nucleotides in length. As a non-limiting example, the viral genome comprises a promoter region that is about 219 nucleotides in length. As a non-limiting example, the viral genome comprises a promoter region that is about 260 nucleotides in length. As a non-limiting example, the viral genome comprises a promoter region that is about 303 nucleotides in length. As a non-limiting example, the viral genome comprises a promoter region that is about 382 nucleotides in length. As a non-limiting example, the viral genome comprises a promoter region that is about 588 nucleotides in length.

In one embodiment, the AAV particle viral genome comprises at least one promoter sequence region. Non-limiting examples of promoter sequence regions are described in Table 21.

TABLE 21
Promoter Sequence Regions

| Sequence Region Name | SEQ ID NO or Sequence |
|---|---|
| Promoter1 | 1816 |
| Promoter2 | 1817 |
| Promoter3 | GTTG |
| Promoter4 | 1818 |
| Promoter5 | 1819 |
| Promoter6 | 1820 |

In one embodiment, the AAV particle viral genome comprises one promoter sequence region. In one embodiment, the promoter sequence region is Promoter1. In one embodiment, the promoter sequence region is Promoter2. In one embodiment, the promoter sequence region is Promoter3. In one embodiment, the promoter sequence region is Promoter4. In one embodiment, the promoter sequence region is Promoter5. In one embodiment, the promoter sequence region is Promoter6.

In one embodiment, the AAV particle viral genome comprises two promoter sequence regions. In one embodiment, the promoter sequence region is Promoter1 sequence region, and the Promoter2 sequence region. In one embodiment, the promoter sequence region is Promoter1 sequence region, and the Promoter3 sequence region. In one embodiment, the promoter sequence region is Promoter1 sequence region, and the Promoter4 sequence region. In one embodiment, the promoter sequence region is Promoter1 sequence region, and the Promoter5 sequence region. In one embodiment, the promoter sequence region is Promoter1 sequence region, and the Promoter6 sequence region. In one embodiment, the promoter sequence region is Promoter2 sequence region, and the Promoter3 sequence region. In one embodiment, the promoter sequence region is Promoter2 sequence region, and the Promoter4 sequence region. In one embodiment, the promoter sequence region is Promoter2 sequence region, and the Promoter5 sequence region. In one embodiment, the promoter sequence region is Promoter2 sequence region, and the Promoter6 sequence region. In one embodiment, the promoter sequence region is Promoter3 sequence region, and the Promoter4 sequence region. In one embodiment, the promoter sequence region is Promoter3 sequence region, and the Promoter5 sequence region. In one embodiment, the promoter sequence region is Promoter3 sequence region, and the Promoter6 sequence region. In one embodiment, the promoter sequence region is Promoter4 sequence region, and the Promoter5 sequence region. In one embodiment, the promoter sequence region is Promoter4 sequence region, and the Promoter6 sequence region. In one embodiment, the promoter sequence region is Promoter5 sequence region, and the Promoter6 sequence region.

In one embodiment, the AAV particle viral genome may comprise at least one exon sequence region. The exon region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the exon region for the viral genome may be 2-10, 5-10, 5-15, 10-20, 10-30, 10-40, 15-20, 15-25, 20-30, 20-40, 20-50, 25-30, 25-35, 30-40, 30-50, 30-60, 35-40, 35-45, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides. As a non-limiting example, the viral genome comprises an exon region that is about 53 nucleotides in length. As a non-limiting example, the viral genome comprises an exon region that is about 134 nucleotides in length.

In one embodiment, the AAV particle viral genome comprises at least one Exon sequence region. Non-limiting examples of Exon sequence regions are described in Table 22.

TABLE 22
Exon Sequence Regions

| Sequence Region Name | SEQ ID NO |
|---|---|
| Exon1 | 1821 |
| Exon2 | 1822 |

In one embodiment, the AAV particle viral genome comprises one Exon sequence region. In one embodiment, the Exon sequence regions is the Exon1 sequence region. In one embodiment, the Exon sequence regions is the Exon2 sequence region.

In one embodiment, the AAV particle viral genome comprises two Exon sequence regions. In one embodiment, the Exon sequence regions are the Exon1 sequence region and the Exon 2 sequence region.

In one embodiment, the AAV particle viral genome may comprise at least one intron sequence region. The intron region(s) may, independently, have a length such as, but not limited to, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, and 350 nucleotides. The length of the intron region for the viral genome may be 25-35, 25-50, 35-45, 45-55, 50-75, 55-65, 65-75, 75-85, 75-100, 85-95, 95-105, 100-125, 105-115, 115-125, 125-135, 125-150, 135-145, 145-155, 150-175, 155-165, 165-175, 175-185, 175-200, 185-195, 195-205, 200-225, 205-215, 215-225, 225-235, 225-250, 235-245, 245-255, 250-275, 255-265, 265-275, 275-285, 275-300, 285-295, 295-305, 300-325, 305-315, 315-325, 325-335, 325-350, and 335-345 nucleotides. As a non-limiting example, the viral genome comprises an intron region that is about 32 nucleotides in length. As a non-limiting example, the viral genome comprises an intron region that is about 172 nucleotides in length. As a non-limiting example, the viral genome comprises an intron region that is about 201 nucleotides in length. As a non-limiting example, the viral genome comprises an intron region that is about 347 nucleotides in length.

In one embodiment, the AAV particle viral genome comprises at least one intron sequence region. Non-limiting examples of intron sequence regions are described in Table 23.

TABLE 23
Intron Sequence Regions

| Sequence Region Name | SEQ ID NO |
|---|---|
| Intron1 | 1823 |
| Intron2 | 1824 |
| Intron3 | 1825 |
| Intron4 | 1826 |

In one embodiment, the AAV particle viral genome comprises one intron sequence region. In one embodiment, the intron sequence regions is the Intron1 sequence region. In one embodiment, the intron sequence regions is the Intron2 sequence region. In one embodiment, the intron sequence regions is the Intron3 sequence region. In one embodiment, the intron sequence regions is the Intron4 sequence region.

In one embodiment, the AAV particle viral genome comprises two intron sequence regions. In one embodiment, the intron sequence regions are the Intron1 sequence region and the Intron2 sequence region. In one embodiment, the intron sequence regions are the Intron1 sequence region and the Intron3 sequence region. In one embodiment, the intron sequence regions are the Intron1 sequence region and the Intron4 sequence region. In one embodiment, the intron sequence regions are the Intron2 sequence region and the Intron3 sequence region. In one embodiment, the intron sequence regions are the Intron2 sequence region and the Intron4 sequence region. In one embodiment, the intron sequence regions are the Intron3 sequence region and the Intron4 sequence region.

In one embodiment, the AAV particle viral genome comprises three intron sequence regions. In one embodiment, the intron sequence regions are the Intron1 sequence region, the Intron2 sequence region, and the Intron3 sequence region. In one embodiment, the intron sequence regions are the Intron1 sequence region, the Intron2 sequence region, and the Intron4 sequence region. In one embodiment, the intron sequence regions are the Intron1 sequence region, the Intron3 sequence region, and the Intron4 sequence region. In one embodiment, the intron sequence regions are the Intron2 sequence region, the Intron3 sequence region, and the Intron4 sequence region.

In one embodiment, the AAV particle viral genome may comprise at least one polyadenylation signal sequence region. The polyadenylation signal region sequence region(s) may, independently, have a length such as, but not limited to, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 nucleotides. The length of the polyadenylation signal sequence region for the viral genome may be 4-10, 10-20, 10-50, 20-30, 30-40, 40-50, 50-60, 50-100, 60-70, 70-80, 80-90, 90-100, 100-110, 100-150, 110-120, 120-130, 130-140, 140-150, 150-160, 150-200, 160-170, 170-180, 180-190, 190-200, 200-210, 200-250, 210-220, 220-230, 230-240, 240-250, 250-260, 250-300, 260-270, 270-280, 280-290, 290-300, 300-310, 300-350, 310-320, 320-330, 330-340, 340-350, 350-360, 350-400, 360-370, 370-380, 380-390, 390-400, 400-410, 400-450, 410-420, 420-430, 430-440, 440-450, 450-460, 450-500, 460-470, 470-480, 480-490, 490-500, 500-510, 500-550, 510-520, 520-530, 530-540, 540-550, 550-560, 550-600, 560-570, 570-580, 580-590, and 590-600 nucleotides. As a non-limiting example, the viral genome comprises a polyadenylation signal sequence region that is about 127 nucleotides in length. As a non-limiting example, the viral genome comprises a polyadenylation signal sequence region that is about 225 nucleotides in length. As a non-limiting example, the viral genome comprises a polyadenylation signal sequence region that is about 476 nucleotides in length. As a non-limiting example, the viral genome comprises a polyadenylation signal sequence region that is about 477 nucleotides in length.

In one embodiment, the AAV particle viral genome comprises at least one polyadenylation (polyA) signal sequence region. Non-limiting examples of polyA signal sequence regions are described in Table 24.

TABLE 24
PolyA Signal Sequence Regions

| Sequence Region Name | SEQ ID NO |
|---|---|
| PolyA1 | 1827 |
| PolyA2 | 1828 |
| PolyA3 | 1829 |
| PolyA4 | 1830 |

In one embodiment, the AAV particle viral genome comprises one polyA signal sequence region. In one embodiment, the polyA signal sequence regions is the PolyA1 sequence region. In one embodiment, the polyA signal sequence regions is the PolyA2 sequence region. In one embodiment, the polyA signal sequence regions is the PolyA3 sequence region. In one embodiment, the polyA signal sequence regions is the PolyA4 sequence region.

In one embodiment, the AAV particle viral genome comprises more than one polyA signal sequence region.

AAV particles may be modified to enhance the efficiency of delivery. Such modified AAV particles comprising the nucleic acid sequence encoding the siRNA molecules of the present invention can be packaged efficiently and can be used to successfully infect the target cells at high frequency and with minimal toxicity.

In some embodiments, the AAV particle comprising a nucleic acid sequence encoding the siRNA molecules of the present invention may be a human serotype AAV particle. Such human AAV particle may be derived from any known serotype, e.g., from any one of serotypes AAV1-AAV11. As non-limiting examples, AAV particles may be vectors comprising an AAV1-derived genome in an AAV1-derived capsid; vectors comprising an AAV2-derived genome in an AAV2-derived capsid; vectors comprising an AAV4-derived genome in an AAV4 derived capsid; vectors comprising an AAV6-derived genome in an AAV6 derived capsid or vectors comprising an AAV9-derived genome in an AAV9 derived capsid.

In other embodiments, the AAV particle comprising a nucleic acid sequence for encoding siRNA molecules of the present invention may be a pseudotyped hybrid or chimeric AAV particle which contains sequences and/or components originating from at least two different AAV serotypes. Pseudotyped AAV particles may be vectors comprising an AAV genome derived from one AAV serotype and a capsid protein derived at least in part from a different AAV serotype. As non-limiting examples, such pseudotyped AAV particles may be vectors comprising an AAV2-derived genome in an AAV1-derived capsid; or vectors comprising an AAV2-derived genome in an AAV6-derived capsid; or vectors comprising an AAV2-derived genome in an AAV4-derived capsid; or an AAV2-derived genome in an AAV9-derived capsid. In like fashion, the present invention contemplates any hybrid or chimeric AAV particle.

In other embodiments, AAV particles comprising a nucleic acid sequence encoding the siRNA molecules of the present invention may be used to deliver siRNA molecules to the central nervous system (e.g., U.S. Pat. No. 6,180,613; the contents of which is herein incorporated by reference in its entirety).

In some aspects, the AAV particles comprising a nucleic acid sequence encoding the siRNA molecules of the present invention may further comprise a modified capsid including peptides from non-viral origin. In other aspects, the AAV particle may contain a CNS specific chimeric capsid to facilitate the delivery of encoded siRNA duplexes into the brain and the spinal cord. For example, an alignment of cap nucleotide sequences from AAV variants exhibiting CNS tropism may be constructed to identify variable region (VR) sequence and structure.

### Polycistronic AAV Particles Comprising Modulatory Polynucleotides

In one embodiment, the AAV vector comprises a nucleic acid sequence encoding more than one modulatory polynucleotide. In one embodiment, the AAV vector comprises a nucleic acid sequence encoding more than one siRNA molecule. The AAV vector may comprise a nucleic acid sequence encoding 2, 3, 4, 5, 6, 7, 8, 9 or more than 9 modulatory polynucleotides. The AAV vector may comprise a nucleic acid sequence encoding 2, 3, 4, 5, 6, 7, 8, 9 or more than 9 siRNA molecules.

When the AAV vector comprises at least one nucleic acid sequence encoding more than one modulatory polynucleotide, e.g., siRNA molecule, the AAV vector may be referred to as polycistronic. When the nucleic acid sequence of the AAV vector encodes modulatory polynucleotide molecules, e.g., siRNA molecules, targeting a single target, then the AAV vector may be referred to as a “monospecific polycistronic” AAV vector. When the nucleic acid sequence of the AAV vector encodes modulatory polynucleotide molecules, e.g., siRNA molecules, targeting more than one target, then the AAV vector may be referred to as a “multispecific polycistronic” AAV vector. When the nucleic acid sequence of the AAV vector encodes siRNA molecules targeting two targets then the AAV vector may be referred to as a “bispecific polycistronic” AAV vector.

In one embodiment, the AAV vector comprises at least one nucleic acid sequence encoding a modulatory polynucleotide, e.g., siRNA molecule, targeting a single target gene. The AAV vector may comprise 1, 2, 3, 4, 5, 6, 7, 8, 9 or more than 9 nucleic acid sequences encoding a single modulatory polynucleotide, e.g., siRNA molecule, targeting a single target gene. As a non-limiting example, the target gene is HTT. As another non-limiting example, the target gene is SOD1.

In one embodiment, the AAV vector is a monospecific polycistronic AAV vector and comprises a nucleic acid sequence encoding two modulatory polynucleotides, e.g., siRNA molecules, targeting a target gene. In one aspect, the modulatory polynucleotides, e.g., siRNA molecules, comprise the same sense strands. In another aspect, the modulatory polynucleotides, e.g., siRNA molecules, comprise different sense strands. In one aspect, the modulatory polynucleotides, e.g., siRNA molecules, comprise different sense strands which have at least 80% complementarity (e.g., 80%, 85%, 90%, 95%, 99% or more than 99%, 80-85%, 80-90%, 85-90%, 85-95%, 90-95%, 90-100%) to the same region on the target gene sequence. In one aspect, the modulatory polynucleotides, e.g., siRNA molecules, comprise different sense strands which have complementarity to different regions of the target gene sequence. As a non-limiting example, the target gene is HTT. As another non-limiting example, the target gene is SOD1.

In one embodiment, the AAV vector is a monospecific polycistronic AAV vector and comprises a nucleic acid sequence encoding three modulatory polynucleotides, e.g., siRNA molecules, targeting a target gene. In one aspect, the modulatory polynucleotides, e.g., siRNA molecules, comprise the same sense strands. In another aspect, each of the modulatory polynucleotides, e.g., siRNA molecules, comprise different sense strands. In another aspect, two of the modulatory polynucleotides, e.g., siRNA molecules, comprise the same sense strand and the third modulatory polynucleotide, e.g., siRNA molecule, comprises a different sense strand. In one aspect, each of the modulatory polynucleotides, e.g., siRNA molecules, comprise different sense strands which have at least 80% complementarity (e.g., 80%, 85%, 90%, 95%, 99% or more than 99%, 80-85%, 80-90%, 85-90%, 85-95%, 90-95%, 90-100%) to the same region on the target gene sequence. In one aspect, two of the modulatory polynucleotides, e.g., siRNA molecules, comprise different sense strands which have at least 80% complementarity (e.g., 80%, 85%, 90%, 95%, 99% or more than 99%, 80-85%, 80-90%, 85-90%, 85-95%, 90-95%, 90-100%) to the same region on the target gene sequence. In one aspect, the modulatory polynucleotides, e.g., siRNA molecules, comprise different sense strands which have complementarity to different regions of the target gene sequence. As a non-limiting example, the target gene is HTT. As another non-limiting example, the target gene is SOD1.

In one embodiment, the AAV vector is a monospecific polycistronic AAV vector and comprises a nucleic acid sequence encoding four modulatory polynucleotides, e.g., siRNA molecules, targeting a target gene. In one aspect, the modulatory polynucleotides, e.g., siRNA molecules, comprise the same sense strands. In another aspect, each of the modulatory polynucleotides, e.g., siRNA molecules, comprise different sense strands. In another aspect, two of the modulatory polynucleotides, e.g., siRNA molecules, comprise a first sense strand sequence and the other two modulatory polynucleotides, e.g., siRNA molecules, comprise a second sense strand sequence. In another aspect, three of the modulatory polynucleotides, e.g., siRNA molecules, comprise a first sense strand sequence and the other modulatory polynucleotides e.g., siRNA molecule, comprises a second sense strand sequence. In one aspect, each of the modulatory polynucleotides, e.g., siRNA molecules, comprise different sense strands which have at least 80% complementarity (e.g., 80%, 85%, 90%, 95%, 99% or more than 99%, 80-85%, 80-90%, 85-90%, 85-95%, 90-95%, 90-100%) to the same region on the target gene sequence. In one aspect, two of the modulatory polynucleotides, e.g., siRNA molecules, comprise different sense strands which have at least 80% complementarity (e.g., 80%, 85%, 90%, 95%, 99% or more than 99%, 80-85%, 80-90%, 85-90%, 85-95%, 90-95%, 90-100%) to the same region on the target gene sequence. In one aspect, the modulatory polynucleotides, e.g., siRNA molecules, comprise different sense strands which have complementarity to different regions of the target gene sequence. As a non-limiting example, the target gene is HTT. As another non-limiting example, the target gene is SOD1.

In one embodiment, the AAV particle is a bispecific polycistronic AAV particle and comprises a nucleic acid sequence encoding two modulatory polynucleotides, e.g., siRNA molecules. In one aspect, one of the modulatory polynucleotides, e.g., siRNA molecules, targets a first target gene and the other modulatory polynucleotide, e.g., siRNA molecule, targets a second target gene, and may reduce the expression of a protein and/or mRNA in at least one region of the central nervous system to treat a disease or disorder of the central nervous system. As a non-limiting example, the target genes are HTT and SOD1 and the diseases are HD and ALS.

In one embodiment, the AAV particle is a multispecific polycistronic AAV particle and comprises a nucleic acid sequence encoding two or more modulatory polynucleotides, e.g., siRNA molecules. In one aspect, one of the modulatory polynucleotides, e.g., siRNA molecules, targets a first target gene and the other modulatory polynucleotide(s), e.g., siRNA molecule(s), targets a second target gene and may reduce the expression of a protein and/or mRNA in at least one region of the central nervous system to treat a disease or disorder of the central nervous system. In one aspect, each of the modulatory polynucleotides, e.g., siRNA molecules, target a different mRNA to reduce the expression of a protein and/or mRNA in at least one region of the central nervous system to treat a disease or disorder of the central nervous system. As a non-limiting example, the target genes are HTT and SOD1 and the diseases are HD and ALS.

In one embodiment, the AAV particle may comprise modulatory polynucleotides comprising more than one molecular scaffold sequence. The AAV particle may comprise 1, 2, 3, 4, 5, 6, 7, 8, 9, or more than 9 molecular scaffold sequences.

In one embodiment, the polycistronic AAV particle viral genome comprises at least one inverted terminal repeat (ITR) sequence region, at least one enhancer sequence region, at least one promoter sequence region, two modulatory polynucleotide regions, and at least one polyadenylation signal sequence region.

In one embodiment, the polycistronic AAV particle viral genome comprises a 5′ inverted terminal repeat (ITR) sequence region and a 3′ ITR sequence region, a CMV enhancer sequence region, a CBA promoter sequence region, two modulatory polynucleotide sequence regions targeting the same gene of interest (HTT), and a rabbit globin polyadenylation signal sequence region. Non-limiting examples of an ITR to ITR sequence for use in the polycistronic AAV particles of the present invention having all of the sequence modules above are described in Table 25. In Table 25, the sequence identifier or sequence of the sequence region (Region SEQ ID NO) and the length of the sequence region (Region length) are described as well as the name and sequence identifier of the ITR to ITR sequence (e.g., VOYPC1 (SEQ ID NO: 1831)).

TABLE 25
Sequence Regions in ITR to ITR Sequence

| | |
|---|---|
| Sequence | VOYPC1 (SEQ ID NO: 1831) |

| Regions | Region SEQ ID NO | Region length |
|---|---|---|
| 5′ ITR | 1788 | 105 |
| CMV enhancer | 1814 | 382 |
| CBA Promoter | 1816 | 260 |
| Modulatory | 1595 | 158 |
| Polynucleotide | | |
| (VOYHTmiR-104.579) | | |
| Modulatory | 1595 | 158 |
| Polynucleotide | | |
| (VOYHTmiR-104.579) | | |
| Rabbit globin | 1827 | 127 |
| PolyA Signal | | |
| 3′ ITR | 1790 | 130 |

In one embodiment, the polycistronic AAV particle viral genome comprises SEQ ID NO: 1831 (VOYPC1) which comprises a 5′ inverted terminal repeat (ITR) sequence region and a 3′ ITR sequence region, a CMV enhancer sequence region, a CBA promoter sequence region, two modulatory polynucleotide regions targeting the same gene of interest (HTT), and a rabbit globin polyadenylation signal sequence region.

In one embodiment, the polycistronic AAV particle viral genome comprises at least one inverted terminal repeat (ITR) sequence region, at least one enhancer sequence region, at least one promoter sequence region, at least one intron sequence region, two modulatory polynucleotide regions, and at least one polyadenylation signal sequence region.

In one embodiment, the polycistronic AAV particle viral genome comprises a 5′ inverted terminal repeat (ITR) sequence region and a 3′ ITR sequence region, a CMV enhancer sequence region, a CBA promoter sequence region, a SV40 intron sequence region, two modulatory polynucleotide sequence regions targeting the same gene of interest (HTT), and a rabbit globin polyadenylation signal sequence region. Non-limiting examples of an ITR to ITR sequence for use in the polycistronic AAV particles of the present invention having all of the sequence modules above are described in Tables 26 and 27. In Table 26 and 27, the sequence identifier or sequence of the sequence region (Region SEQ ID NO) and the length of the sequence region (Region length) are described as well as the name and sequence identifier of the ITR to ITR sequence (e.g., VOYPC2 (SEQ ID NO: 1832)).

TABLE 26
Sequence Regions in ITR to ITR Sequences

| | VOYPC2 | | VOYPC3 | | VOYPC4 | | VOYPC5 | |
|---|---|---|---|---|---|---|---|---|
| | (SEQ ID NO: 1832) | | (SEQ ID NO: 1833) | | (SEQ ID NO: 1834) | | (SEQ ID NO: 1835) | |
| Sequence | Region | Region | Region | Region | Region | Region | Region | Region |
| Regions | SEQ ID NO | length | SEQ ID NO | length | SEQ ID NO | length | SEQ ID NO | length |
| 5′ ITR | 1788 | 105 | 1788 | 105 | 1788 | 105 | 1788 | 105 |
| CMV enhancer | 1814 | 382 | 1814 | 382 | 1814 | 382 | 1814 | 382 |
| CBA Promoter | 1816 | 260 | 1816 | 260 | 1816 | 260 | 1816 | 260 |
| SV40 Intron | 1823 | 172 | 1823 | 172 | 1823 | 172 | 1823 | 172 |
| Modulatory | 1589 | 158 | — | — | 1589 | 158 | — | — |
| Polynucleotide | | | | | | | | |
| (VOYHTmiR-104.016) | | | | | | | | |
| Modulatory | — | — | 1599 | 260 | — | — | 1599 | 260 |
| Polynucleotide | | | | | | | | |
| (VOYHTmiR-127.579) | | | | | | | | |
| Modulatory | 1589 | 158 | 1589 | 158 | — | — | — | — |
| Polynucleotide | | | | | | | | |
| (VOYHTmiR-104.016) | | | | | | | | |
| Modulatory | — | — | — | — | 1599 | 260 | 1599 | 260 |
| Polynucleotide | | | | | | | | |
| (VOYHTmiR-127.579) | | | | | | | | |
| Rabbit globin | 1827 | 127 | 1827 | 127 | 1827 | 127 | 1827 | 127 |
| PolyA Signal | | | | | | | | |
| 3′ ITR | 1790 | 130 | 1790 | 130 | 1790 | 130 | 1790 | 130 |

TABLE 27
Sequence Regions in ITR to ITR Sequences

| | VOYPC6 (SEQ ID NO: 1836) | | VOYPC7 (SEQ ID NO: 1837) | | VOYPC8 (SEQ ID NO: 1838) | |
|---|---|---|---|---|---|---|
| Sequence | Region | Region | Region | Region | Region | Region |
| Regions | SEQ ID NO | length | SEQ ID NO | length | SEQ ID NO | length |
| 5′ ITR | 1788 | 105 | 1788 | 105 | 1788 | 105 |
| CMV enhancer | 1814 | 382 | 1814 | 382 | 1814 | 382 |
| CBA Promoter | 1816 | 260 | 1816 | 260 | 1816 | 260 |
| SV40 Intron | 1826 | 201 | 1826 | 201 | 1826 | 201 |
| Modulatory | 1593 | 260 | — | — | 1593 | 260 |
| Polynucleotide | | | | | | |
| (VOYHTmiR-127.016) | | | | | | |
| Modulatory | — | — | 1595 | 158 | — | — |
| Polynucleotide | | | | | | |
| (VOYHTmiR-104.579) | | | | | | |
| Modulatory | 1593 | 260 | 1593 | 260 | — | — |
| Polynucleotide | | | | | | |
| (VOYHTmiR-127.016) | | | | | | |
| Modulatory | — | — | — | — | 1595 | 158 |
| Polynucleotide | | | | | | |
| (VOYHTmiR-104.579) | | | | | | |
| Rabbit globin | 1827 | 127 | 1827 | 127 | 1827 | 127 |
| PolyA Signal | | | | | | |
| 3′ ITR | 1790 | 130 | 1790 | 130 | 1790 | 130 |

In one embodiment, the polycistronic AAV particle viral genome comprises SEQ ID NO: 1832 (VOYPC2) which comprises a 5′ inverted terminal repeat (ITR) sequence region and a 3′ ITR sequence region, a CMV enhancer sequence region, a CBA promoter sequence region, a SV40 intron sequence region, two modulatory polynucleotide regions targeting the same gene of interest (HTT), and a rabbit globin polyadenylation signal sequence region.

In one embodiment, the polycistronic AAV particle viral genome comprises SEQ ID NO: 1833 (VOYPC3) which comprises a 5′ inverted terminal repeat (ITR) sequence region and a 3′ ITR sequence region, a CMV enhancer sequence region, a CBA promoter sequence region, a SV40 intron sequence region, two modulatory polynucleotide regions targeting the same gene of interest (HTT), and a rabbit globin polyadenylation signal sequence region.

In one embodiment, the polycistronic AAV particle viral genome comprises SEQ ID NO: 1834 (VOYPC4) which comprises a 5′ inverted terminal repeat (ITR) sequence region and a 3′ ITR sequence region, a CMV enhancer sequence region, a CBA promoter sequence region, a SV40 intron sequence region, two modulatory polynucleotide regions targeting the same gene of interest (HTT), and a rabbit globin polyadenylation signal sequence region.

In one embodiment, the polycistronic AAV particle viral genome comprises SEQ ID NO: 1835 (VOYPC5) which comprises a 5′ inverted terminal repeat (ITR) sequence region and a 3′ ITR sequence region, a CMV enhancer sequence region, a CBA promoter sequence region, a SV40 intron sequence region, two modulatory polynucleotide regions targeting the same gene of interest (HTT), and a rabbit globin polyadenylation signal sequence region.

In one embodiment, the polycistronic AAV particle viral genome comprises SEQ ID NO: 1836 (VOYPC6) which comprises a 5′ inverted terminal repeat (ITR) sequence region and a 3′ ITR sequence region, a CMV enhancer sequence region, a CBA promoter sequence region, a SV40 intron sequence region, two modulatory polynucleotide regions targeting the same gene of interest (HTT), and a rabbit globin polyadenylation signal sequence region.

In one embodiment, the polycistronic AAV particle viral genome comprises SEQ ID NO: 1837 (VOYPC7) which comprises a 5′ inverted terminal repeat (ITR) sequence region and a 3′ ITR sequence region, a CMV enhancer sequence region, a CBA promoter sequence region, a SV40 intron sequence region, two modulatory polynucleotide regions targeting the same gene of interest (HTT), and a rabbit globin polyadenylation signal sequence region.

In one embodiment, the polycistronic AAV particle viral genome comprises SEQ ID NO: 1838 (VOYPC8) which comprises a 5′ inverted terminal repeat (ITR) sequence region and a 3′ ITR sequence region, a CMV enhancer sequence region, a CBA promoter sequence region, a SV40 intron sequence region, two modulatory polynucleotide regions targeting the same gene of interest (HTT), and a rabbit globin polyadenylation signal sequence region

In one embodiment, the polycistronic AAV particle viral genome comprises at least one inverted terminal repeat (ITR) sequence region, at least one promoter sequence region, two modulatory polynucleotide regions, and at least one polyadenylation signal sequence region.

In one embodiment, the polycistronic AAV particle viral genome comprises at least one inverted terminal repeat (ITR) sequence region, at least one promoter sequence region, and two modulatory polynucleotide regions.

In one embodiment, the polycistronic AAV particle viral genome comprises a 5′ inverted terminal repeat (ITR) sequence region and a 3′ ITR sequence region a CBA promoter sequence region, a H1 promoter sequence region, and two modulatory polynucleotide sequence regions targeting the same gene of interest (HTT). Non-limiting examples of an ITR to ITR sequence for use in the polycistronic AAV particles of the present invention having all of the sequence modules above are described in Table 28. In Table 28, the sequence identifier or sequence of the sequence region (Region SEQ ID NO) and the length of the sequence region (Region length) are described as well as the name and sequence identifier of the ITR to ITR sequence (e.g., VOYPC1 (SEQ ID NO: 1831)).

TABLE 28
Sequence Regions in ITR to ITR Sequences

| | VOYPC9 | | VOYPC10 | | VOYPC11 | | VOYPC12 | |
|---|---|---|---|---|---|---|---|---|
| | (SEQ ID NO: 1839) | | (SEQ ID NO: 1840) | | (SEQ ID NO: 1841) | | (SEQ ID NO: 1842) | |
| Sequence | Region | Region | Region | Region | Region | Region | Region | Region |
| Regions | SEQ ID NO | length | SEQ ID NO | length | SEQ ID NO | length | SEQ ID NO | length |
| 5′ ITR | 1788 | 105 | 1788 | 105 | 1788 | 105 | 1788 | 105 |
| CBA Promoter | 1819 | 219 | 1819 | 219 | 1819 | 219 | 1819 | 219 |
| Modulatory | 1589 | 158 | — | — | — | — | 1589 | 158 |
| Polynucleotide | | | | | | | | |
| (VOYHTmiR-104.016) | | | | | | | | |
| Modulatory | — | — | 1599 | 260 | 1599 | 260 | — | — |
| Polynucleotide | | | | | | | | |
| (VOYHTmiR-127.579) | | | | | | | | |
| H1 Promoter | 1819 | 219 | 1819 | 219 | 1819 | 219 | 1819 | 219 |
| Modulatory | 1589 | 158 | — | — | 1589 | 158 | — | — |
| Polynucleotide | | | | | | | | |
| (VOYHTmiR-104.016) | | | | | | | | |
| Modulatory | — | — | 1599 | 260 | — | — | 1599 | 260 |
| Polynucleotide | | | | | | | | |
| (VOYHTmiR-127.579) | | | | | | | | |
| 3′ ITR | 1790 | 130 | 1790 | 130 | 1790 | 130 | 1790 | 130 |

In one embodiment, the polycistronic AAV particle viral genome comprises a Pol III promoter. In one embodiment, the polycistronic AAV particle viral genome comprises a type 3 Pol III promoter. In one embodiment, the polycistronic AAV particle viral genome comprises a H1 promoter. In one embodiment, the polycistronic AAV particle viral genome comprises a U6 promoter. In one embodiment, the polycistronic AAV particle viral genome comprises a U3 promoter. In one embodiment, the polycistronic AAV particle viral genome comprises a U7 promoter. In one embodiment, the polycistronic AAV particle viral genome comprises a 7SK promoter. In one embodiment, the polycistronic AAV particle viral genome comprises an MRP promoter. In one embodiment, the polycistronic AAV particle viral genome comprises a Pol II promoter. In one embodiment, the polycistronic AAV particle viral genome comprises a truncated Pol II promoter.

In one embodiment, the polycistronic AAV particle viral genome comprises SEQ ID NO: 1839 (VOYPC9) which comprises a 5′ inverted terminal repeat (ITR) sequence region and a 3′ ITR sequence region, a CBA promoter sequence region, a H1 promoter sequence region, and two modulatory polynucleotide regions targeting the same gene of interest (HTT).

In one embodiment, the polycistronic AAV particle viral genome comprises SEQ ID NO: 1840 (VOYPC10) which comprises a 5′ inverted terminal repeat (ITR) sequence region and a 3′ ITR sequence region, a CBA promoter sequence region, a H1 promoter sequence region, and two modulatory polynucleotide regions targeting the same gene of interest (HTT).

In one embodiment, the polycistronic AAV particle viral genome comprises SEQ ID NO: 1841 (VOYPC11) which comprises a 5′ inverted terminal repeat (ITR) sequence region and a 3′ ITR sequence region, a CBA promoter sequence region, a H1 promoter sequence region, and two modulatory polynucleotide regions targeting the same gene of interest (HTT).

In one embodiment, the polycistronic AAV particle viral genome comprises SEQ ID NO: 1842 (VOYPC12) which comprises a 5′ inverted terminal repeat (ITR) sequence region and a 3′ ITR sequence region, a CBA promoter sequence region, a H1 promoter sequence region, and two modulatory polynucleotide regions targeting the same gene of interest (HTT).

In one embodiment, the polycistronic AAV particle viral genome comprises a 5′ inverted terminal repeat (ITR) sequence region and a 3′ ITR sequence region, two H1 promoter sequence regions, two modulatory polynucleotide sequence regions targeting the same gene of interest, and two H1 terminator sequences, where each modulatory polynucleotide sequence region is driven by its own Pol III promoter, for example, type 3 Pol III promoter, e.g., H1 promoter, and followed by its own promoter terminator sequence, e.g., H1 terminator sequence. Non-limiting examples of an ITR to ITR sequence for use in the polycistronic AAV particles of the present invention having these sequence modules are described in Table 29. In Table 29, the sequence identifier or sequence of the sequence region (Region SEQ ID NO) and the length of the sequence region (Region length) are described as well as the name and sequence identifier of the ITR to ITR sequence (e.g., VOYPC59 (SEQ ID NO: 2682))

TABLE 29
Sequence Regions in ITR to ITR Sequences

| | VOYPC59 | | VOYPC60 | | VOYPC61 | | VOYPC62 | |
|---|---|---|---|---|---|---|---|---|
| | (SEQ ID NO: 2682) | | (SEQ ID NO: 2683) | | (SEQ ID NO: 2684) | | (SEQ ID NO: 2685) | |
| Sequence | Region | Region | Region | Region | Region | Region | Region | Region |
| Regions | SEQ ID NO | length | SEQ ID NO | length | SEQ ID NO | length | SEQ ID NO | length |
| 5′ ITR | 1788 | 105 | 1788 | 105 | 1788 | 105 | 1788 | 105 |
| H1 Promoter | 1819 | 219 | 1819 | 219 | | | | |
| Modulatory | 1589 | 158 | 1589 | 158 | | | | |
| Polynucleotide | | | | | | | | |
| (VOYHTmiR-104.016) | | | | | | | | |
| H1 Terminator | 2681 | 5 | 2681 | 5 | | | | |
| H1 Promoter | | | 1819 | 219 | 1819 | 219 | 1819 | 219 |
| Modulatory | | | 1599 | 260 | 1599 | 260 | 1599 | 260 |
| Polynucleotide | | | | | | | | |
| (VOYHTmiR-127.579) | | | | | | | | |
| H1 Terminator | | | 2681 | 5 | 2681 | 5 | 2681 | 5 |
| H1 Promoter | 1819 | 219 | | | 1819 | 219 | | |
| Modulatory | 1589 | 158 | | | 1589 | 158 | | |
| Polynucleotide | | | | | | | | |
| (VOYHTmiR-104.016) | | | | | | | | |
| H1 Terminator | 2681 | 5 | | | 2681 | 5 | | |
| H1 Promoter | | | | | | | 1819 | 219 |
| Modulatory | | | | | | | 1599 | 260 |
| Polynucleotide | | | | | | | | |
| (VOYHTmiR-127.579) | | | | | | | | |
| H1 Terminator | | | | | | | 2681 | 5 |
| 3′ ITR | 1790 | 130 | 1790 | 130 | 1790 | 130 | 1790 | 130 |

In one embodiment, the polycistronic AAV particle viral genome comprises SEQ ID NO: 2682 (VOYPC59) which comprises a 5′ inverted terminal repeat (ITR) sequence region and a 3′ ITR sequence region, two H1 promoter sequence regions, two modulatory polynucleotide regions targeting the same gene of interest (HTT), and two H1 terminator sequence regions, where each modulatory polynucleotide region is driven by its own H1 promoter and followed by its own H1 terminator.

In one embodiment, the polycistronic AAV particle viral genome comprises SEQ ID NO: 2683 (VOYPC60) which comprises a 5′ inverted terminal repeat (ITR) sequence region and a 3′ ITR sequence region, two H1 promoter sequence regions, two modulatory polynucleotide regions targeting the same gene of interest (HTT), and two H1 terminator sequence regions, where each modulatory polynucleotide region is driven by its own H1 promoter and followed by its own H1 terminator.

In one embodiment, the polycistronic AAV particle viral genome comprises SEQ ID NO: 2684 (VOYPC61) which comprises a 5′ inverted terminal repeat (ITR) sequence region and a 3′ ITR sequence region, two H1 promoter sequence regions, two modulatory polynucleotide regions targeting the same gene of interest (HTT), and two H1 terminator sequence regions, where each modulatory polynucleotide region is driven by its own H1 promoter and followed by its own H1 terminator.

In one embodiment, the polycistronic AAV particle viral genome comprises SEQ ID NO: 2685 (VOYPC62) which comprises a 5′ inverted terminal repeat (ITR) sequence region and a 3′ ITR sequence region, two H1 promoter sequence regions, two modulatory polynucleotide regions targeting the same gene of interest (HTT), and two H1 terminator sequence regions, where each modulatory polynucleotide region is driven by its own H1 promoter and followed by its own H1 terminator.

In one embodiment, the polycistronic AAV particle viral genome comprises two promoter sequence regions, two modulatory polynucleotide regions and at least one polyadenylation sequence region.

In one embodiment, the polycistronic AAV particle viral genome comprises a CMV promoter sequence region, a T7 primer binding site, two modulatory polynucleotide sequence regions targeting the same gene of interest (HTT) and a polyadenylation sequence region. Non-limiting examples of sequences for use in the polycistronic AAV particles of the present invention having all of the sequence modules above are described in Tables 30 and 31. In Tables 30 and 31, the sequence identifier or sequence of the sequence region (Region SEQ ID NO) and the length of the sequence region (Region length) are described as well as the name of the sequence (e.g., VOYPC13).

TABLE 30
Sequence Regions

| | VOYPC13 | | VOYPC14 | | VOYPC15 | | VOYPC16 | |
|---|---|---|---|---|---|---|---|---|
| | (SEQ ID NO: 2686) | | (SEQ ID NO: 2687) | | (SEQ ID NO: 2688) | | (SEQ ID NO: 2689) | |
| Sequence | Region | Region | Region | Region | Region | Region | Region | Region |
| Regions | SEQ ID NO | length | SEQ ID NO | length | SEQ ID NO | length | SEQ ID NO | length |
| CMV Promoter | 1817 | 588 | 1817 | 588 | 1817 | 588 | 1817 | 588 |
| T7 Primer | 1820 | 17 | 1820 | 17 | 1820 | 17 | 1820 | 17 |
| Binding Site | | | | | | | | |
| Modulatory | 1589 | 158 | — | — | 1589 | 158 | — | — |
| Polynucleotide | | | | | | | | |
| (VOYHTmiR-104.016) | | | | | | | | |
| Modulatory | — | — | 1599 | 260 | 1599 | 260 | 1599 | 260 |
| Polynucleotide | | | | | | | | |
| (VOYHTmiR-127.579) | | | | | | | | |
| Modulatory | 1589 | 158 | 1589 | 158 | — | — | — | — |
| Polynucleotide | | | | | | | | |
| (VOYHTmiR-104.016) | | | | | | | | |
| Modulatory | — | — | — | — | — | — | 1599 | 260 |
| Polynucleotide | | | | | | | | |
| (VOYHTmiR-127.579) | | | | | | | | |
| PolyA | 1828 | 225 | 1828 | 225 | 1828 | 225 | 1828 | 225 |

TABLE 31
Sequence Regions

| | VOYPC17 | | VOYPC18 | | VOYPC19 | | VOYPC20 | |
|---|---|---|---|---|---|---|---|---|
| Sequence | Region | Region | Region | Region | Region | Region | Region | Region |
| Regions | SEQ ID NO | length | SEQ ID NO | length | SEQ ID NO | length | SEQ ID NO | length |
| CMV | 1817 | 588 | 1817 | 588 | 1817 | 588 | 1817 | 588 |
| Promoter | | | | | | | | |
| T7 Primer | 1820 | 17 | 1820 | 17 | 1820 | 17 | 1820 | 17 |
| Binding Site | | | | | | | | |
| Modulatory | 1595 | 158 | — | — | 1595 | 158 | — | — |
| Polynucleotide | | | | | | | | |
| (VOYHTmiR-104.579) | | | | | | | | |
| Modulatory | — | — | 1593 | 260 | 1593 | 260 | 1593 | 260 |
| Polynucleotide | | | | | | | | |
| (VOYHTmiR-127.016) | | | | | | | | |
| Modulatory | 1595 | 158 | — | — | — | — | 1595 | 158 |
| Polynucleotide | | | | | | | | |
| (VOYHTmiR-104.579) | | | | | | | | |
| Modulatory | — | — | 1593 | 260 | — | — | — | — |
| Polynucleotide | | | | | | | | |
| (VOYHTmiR-127.016) | | | | | | | | |
| PolyA | 1828 | 225 | 1828 | 225 | 1828 | 225 | 1828 | 225 |

In one embodiment, the polycistronic AAV particle viral genome comprises the sequence modules described in VOYPC13 which comprises a CMV promoter sequence region, a T7 primer binding site, two modulatory polynucleotide regions targeting the same gene of interest (HTT), and a polyadenylation sequence region.

In one embodiment, the polycistronic AAV particle viral genome comprises the sequence modules described in VOYPC14 which comprises a CMV promoter sequence region, a T7 primer binding site, two modulatory polynucleotide regions targeting the same gene of interest (HTT), and a polyadenylation sequence region.

In one embodiment, the polycistronic AAV particle viral genome comprises the sequence modules described in VOYPC15 which comprises a CMV promoter sequence region, a T7 primer binding site, two modulatory polynucleotide regions targeting the same gene of interest (HTT), and a polyadenylation sequence region.

In one embodiment, the polycistronic AAV particle viral genome comprises the sequence modules described in VOYPC16 which comprises a CMV promoter sequence region, a T7 primer binding site, two modulatory polynucleotide regions targeting the same gene of interest (HTT), and a polyadenylation sequence region.

In one embodiment, the polycistronic AAV particle viral genome comprises the sequence modules described in VOYPC17 which comprises a CMV promoter sequence region, a T7 primer binding site, two modulatory polynucleotide regions targeting the same gene of interest (HTT), and a polyadenylation sequence region.

In one embodiment, the polycistronic AAV particle viral genome comprises the sequence modules described in VOYPC18 which comprises a CMV promoter sequence region, a T7 primer binding site, two modulatory polynucleotide regions targeting the same gene of interest (HTT), and a polyadenylation sequence region.

In one embodiment, the polycistronic AAV particle viral genome comprises the sequence modules described in VOYPC19 which comprises a CMV promoter sequence region, a T7 primer binding site, two modulatory polynucleotide regions targeting the same gene of interest (HTT), and a polyadenylation sequence region.

In one embodiment, the polycistronic AAV particle viral genome comprises the sequence modules described in VOYPC20 which comprises a CMV promoter sequence region, a T7 primer binding site, two modulatory polynucleotide regions targeting the same gene of interest (HTT), and a polyadenylation sequence region.

In one embodiment, the polycistronic AAV particle viral genome comprises a CMV promoter sequence region, a T7 primer binding site, and two modulatory polynucleotide sequence regions targeting different genes of interest (HTT and SOD1) and a polyadenylation sequence region. Non-limiting examples of sequences for use in the polycistronic AAV particles of the present invention having all of the sequence modules above are described in Table 32. In Table 32, the sequence identifier or sequence of the sequence region (Region SEQ ID NO) and the length of the sequence region (Region length) are described as well as the name of the sequence (e.g., VOYPC25).

TABLE 32
Sequence Regions

| | VOYPC25 | | VOYPC26 | |
|---|---|---|---|---|
| Sequence | Region | Region | Region | Region |
| Regions | SEQ ID NO | length | SEQ ID NO | length |
| CMV Promoter | 1817 | 588 | 1817 | 588 |
| T7 Primer | 1820 | 17 | 1820 | 17 |
| Binding Site | | | | |
| Modulatory | 1699 | 158 | 1599 | 260 |
| Polynucleotide | | | | |
| (VOYSOD1miR-104) | | | | |
| Modulatory | 1599 | 260 | — | — |
| Polynucleotide | | | | |
| (VOYHTmiR-127.579) | | | | |
| Modulatory | — | — | 1699 | 158 |
| Polynucleotide | | | | |
| (VOYSOD1miR-104) | | | | |
| PolyA | 1828 | 225 | 1828 | 225 |

In one embodiment, the polycistronic AAV particle viral genome comprises the sequence modules described in VOYPC25 which comprises a CMV promoter sequence region, a T7 primer binding site, two modulatory polynucleotide regions targeting the two different genes of interest (HTT and SOD1), and a polyadenylation sequence region.

In one embodiment, the polycistronic AAV particle viral genome comprises the sequence modules described in VOYPC26 which comprises a CMV promoter sequence region, a T7 primer binding site, two modulatory polynucleotide regions targeting the two different genes of interest (HTT and SOD1), and a polyadenylation sequence region.

In one embodiment, the polycistronic AAV particle viral genome comprises three promoter sequence regions, two modulatory polynucleotide regions and at least one polyadenylation sequence region.

In one embodiment, the polycistronic AAV particle viral genome comprises a VTGT region, two H1 promoter sequence region, and two modulatory polynucleotide sequence regions targeting the same gene of interest (HTT). Non-limiting examples of sequences for use in the polycistronic AAV particles of the present invention having all of the sequence modules above are described in Table 33. In Table 33, the sequence identifier or sequence of the sequence region (Region SEQ ID NO) and the length of the sequence region (Region length) are described as well as the name of the sequence (e.g., VOYPC21).

TABLE 33
Sequence Regions

| | VOYPC21 | | VOYPC22 | | VOYPC23 | | VOYPC24 | |
|---|---|---|---|---|---|---|---|---|
| Sequence | Region | Region | Region | Region | Region | Region | Region | Region |
| Regions | SEQ ID NO | length | SEQ ID NO | length | SEQ ID NO | length | SEQ ID NO | length |
| GTTG | — | 4 | — | 4 | — | 4 | — | 4 |
| H1 Promoter | 1819 | 219 | 1819 | 219 | 1819 | 219 | 1819 | 219 |
| Modulatory | 1599 | 260 | 1599 | 260 | — | — | — | — |
| Polynucleotide | | | | | | | | |
| (VOYHTmiR-127.579) | | | | | | | | |
| Modulatory | — | — | — | — | 1589 | 158 | 1589 | 158 |
| Polynucleotide | | | | | | | | |
| (VOYHTmiR-104.016) | | | | | | | | |
| H1 promoter | 1819 | 219 | 1819 | 219 | 1819 | 219 | 1819 | 219 |
| Modulatory | 1599 | 260 | | | 1599 | 260 | — | — |
| Polynucleotide | | | | | | | | |
| (VOYHTmiR-127.579) | | | | | | | | |
| Modulatory | — | — | 1589 | 158 | — | — | 1589 | 158 |
| Polynucleotide | | | | | | | | |
| (VOYHTmiR-104.016) | | | | | | | | |

In one embodiment, the polycistronic AAV particle viral genome comprises the sequence modules described in VOYPC21 which comprises a GTTG region, two H1 promoter sequence regions, and two modulatory polynucleotide regions targeting the same gene of interest (HTT).

In one embodiment, the polycistronic AAV particle viral genome comprises the sequence modules described in VOYPC22 which comprises a GTTG region, two H1 promoter sequence regions, and two modulatory polynucleotide regions targeting the same gene of interest (HTT).

In one embodiment, the polycistronic AAV particle viral genome comprises the sequence modules described in VOYPC23 which comprises a GTTG region, two H1 promoter sequence regions, and two modulatory polynucleotide regions targeting the same gene of interest (HTT).

In one embodiment, the polycistronic AAV particle viral genome comprises the sequence modules described in VOYPC24 which comprises a GTTG region, two H1 promoter sequence regions, and two modulatory polynucleotide regions targeting the same gene of interest (HTT).

In one embodiment, the polycistronic AAV particle viral genome comprises at least one inverted terminal repeat (ITR) sequence region, at least one enhancer sequence region, at least one promoter sequence region, at least one intron sequence region, three modulatory polynucleotide regions, and at least one polyadenylation signal sequence region.

In one embodiment, the polycistronic AAV particle viral genome comprises a 5′ inverted terminal repeat (ITR) sequence region and a 3′ ITR sequence region, a CMV enhancer sequence region, a GTTG region, SV40 intron sequence region, three modulatory polynucleotide sequence regions targeting the same gene of interest (HTT), and a rabbit globin polyadenylation signal sequence region. Non-limiting examples of an ITR to ITR sequence for use in the polycistronic AAV particles of the present invention having all of the sequence modules above are described in Table 34. In Table 34, the sequence identifier or sequence of the sequence region (Region SEQ ID NO) and the length of the sequence region (Region length) are described as well as the name and sequence identifier of the ITR to ITR sequence (e.g., VOYPC27 (SEQ ID NO: 1843)).

TABLE 34
Sequence Regions in ITR to ITR Sequence

| | VOYPC27 | | VOYPC28 | |
|---|---|---|---|---|
| | (SEQ ID NO: 1843) | | (SEQ ID NO: 1844) | |
| Sequence | Region | Region | Region | Region |
| Regions | SEQ ID NO | length | SEQ ID NO | length |
| 5′ ITR | 1788 | 105 | 1788 | 105 |
| CMV enhancer | 1814 | 382 | 1814 | 382 |
| CBA Promoter | 1816 | 260 | 1816 | 260 |
| SV40 intron | 1826 | 201 | 1826 | 201 |
| Modulatory | 1599 | 260 | 1599 | 260 |
| Polynucleotide | | | | |
| (VOYHTmiR-127.579) | | | | |
| Modulatory | 1589 | 158 | 1589 | 158 |
| Polynucleotide | | | | |
| (VOYHTmiR-104.016) | | | | |
| Modulatory | 1589 | 158 | — | — |
| Polynucleotide | | | | |
| (VOYHTmiR-104.016) | | | | |
| Modulatory | — | — | 1599 | 260 |
| Polynucleotide | | | | |
| (VOYHTmiR-127.579) | | | | |
| Rabbit globin | 1827 | 127 | 1827 | 127 |
| PolyA Signal | | | | |
| 3′ ITR | 1790 | 130 | 1790 | 130 |

In one embodiment, the polycistronic AAV particle viral genome comprises SEQ ID NO: 1843 (VOYPC27) which comprises a 5′ inverted terminal repeat (ITR) sequence region and a 3′ ITR sequence region, a CMV enhancer sequence region, a CBA promoter sequence region, a SV40 intron sequence region, three modulatory polynucleotide regions targeting the same gene of interest (HTT), and a rabbit globin polyadenylation signal sequence region.

In one embodiment, the polycistronic AAV particle viral genome comprises SEQ ID NO: 1844 (VOYPC28) which comprises a 5′ inverted terminal repeat (ITR) sequence region and a 3′ ITR sequence region, a CMV enhancer sequence region, a CBA promoter sequence region, a SV40 intron sequence region, three modulatory polynucleotide regions targeting the same gene of interest (HTT), and a rabbit globin polyadenylation signal sequence region.

In one embodiment, the polycistronic AAV particle viral genome comprises at least one inverted terminal repeat (ITR) sequence region, at least one promoter sequence region, and three modulatory polynucleotide regions.

In one embodiment, the polycistronic AAV particle viral genome comprises a 5′ inverted terminal repeat (ITR) sequence region and a 3′ ITR sequence region, three H1 promoter sequence regions, and three modulatory polynucleotide sequence regions targeting the same gene of interest (HTT). Non-limiting examples of an ITR to ITR sequence for use in the polycistronic AAV particles of the present invention having all of the sequence modules above are described in Tables 35 and 36. In Tables 35 and 36, the sequence identifier or sequence of the sequence region (Region SEQ ID NO) and the length of the sequence region (Region length) are described as well as the name and sequence identifier of the ITR to ITR sequence (e.g., VOYPC29 (SEQ ID NO: 1845)).

TABLE 35
Sequence Regions in ITR to ITR Sequence

| | VOYPC29 (SEQ ID NO: 1845) | | VOYPC31 (SEQ ID NO: 1847) | | VOYPC32 (SEQ ID NO: 1848) | |
|---|---|---|---|---|---|---|
| Sequence | Region | Region | Region | Region | Region | Region |
| Regions | SEQ ID NO | length | SEQ ID NO | length | SEQ ID NO | length |
| 5′ ITR | 1788 | 105 | 1788 | 105 | 1788 | 105 |
| H1 Promoter | 1819 | 219 | 1819 | 219 | 1819 | 219 |
| Modulatory | 1599 | 260 | 1599 | 260 | 1599 | 260 |
| Polynucleotide | | | | | | |
| (VOYHTmiR-127.579) | | | | | | |
| H1 Terminator | 2681 | 5 | 2681 | 5 | 2681 | 5 |
| H1 Promoter | 1819 | 219 | 1819 | 219 | 1819 | 219 |
| Modulatory | 1589 | 158 | — | — | — | — |
| Polynucleotide | | | | | | |
| (VOYHTmiR-104.016) | | | | | | |
| Modulatory | — | — | 1599 | 260 | 1599 | 260 |
| Polynucleotide | | | | | | |
| (VOYHTmiR-127.579) | | | | | | |
| H1 Terminator | 2681 | 5 | 2681 | 5 | 2681 | 5 |
| H1 Promoter | 1819 | 219 | 1819 | 219 | 1819 | 219 |
| Modulatory | 1599 | 260 | 1599 | 260 | — | — |
| Polynucleotide | | | | | | |
| (VOYHTmiR-127.579) | | | | | | |
| Modulatory | — | — | — | | 1589 | 158 |
| Polynucleotide | | | | | | |
| (VOYHTmiR-104.016) | | | | | | |
| H1 Terminator | 2681 | 5 | 2681 | 5 | 2681 | 5 |
| 3′ ITR | 1790 | 130 | 1790 | 130 | 1790 | 130 |

TABLE 36
Sequence Regions in ITR to ITR Sequence

| | VOYPC30 (SEQ ID NO: 1846) | | VOYPC33 (SEQ ID NO: 1849) | | VOYPC34 (SEQ ID NO: 1850) | |
|---|---|---|---|---|---|---|
| Sequence | Region | Region | Region | Region | Region | Region |
| Regions | SEQ ID NO | length | SEQ ID NO | length | SEQ ID NO | length |
| 5′ ITR | 1788 | 105 | 1788 | 105 | 1788 | 105 |
| H1 Promoter | 1819 | 219 | 1819 | 219 | 1819 | 219 |
| Modulatory | 1589 | 158 | 1589 | 158 | 1589 | 158 |
| Polynucleotide | | | | | | |
| (VOYHTmiR-104.016) | | | | | | |
| H1 Terminator | 2681 | 5 | 2681 | 5 | 2681 | 5 |
| H1 Promoter | 1819 | 219 | 1819 | 219 | 1819 | 219 |
| Modulatory | 1599 | 260 | — | — | — | — |
| Polynucleotide | | | | | | |
| (VOYHTmiR-127.579) | | | | | | |
| Modulatory | — | — | 1589 | 158 | 1589 | 158 |
| Polynucleotide | | | | | | |
| (VOYHTmiR-104.016) | | | | | | |
| H1 Terminator | 2681 | 5 | 2681 | 5 | 2681 | 5 |
| H1 Promoter | 1819 | 219 | 1819 | 219 | 1819 | 219 |
| Modulatory | | | — | — | 1599 | 260 |
| Polynucleotide | | | | | | |
| (VOYHTmiR-127.579) | | | | | | |
| Modulatory | 1589 | 158 | 1589 | 158 | | |
| Polynucleotide | | | | | | |
| (VOYHTmiR-104.016) | | | | | | |
| H1 Terminator | 2681 | 5 | 2681 | 5 | 2681 | 5 |
| 3′ ITR | 1790 | 130 | 1790 | 130 | 1790 | 130 |

In one embodiment, the polycistronic AAV particle viral genome comprises SEQ ID NO: 1845 (VOYPC29) which comprises a 5′ inverted terminal repeat (ITR) sequence region and a 3′ ITR sequence region, three H1 promoter sequence regions, three modulatory polynucleotide regions targeting the same gene of interest (HTT), and three H1 terminator sequence regions, where each modulatory polynucleotide region is driven by its own H1 promoter and followed by its own H1 terminator.

In one embodiment, the polycistronic AAV particle viral genome comprises SEQ ID NO: 1846 (VOYPC30) which comprises a 5′ inverted terminal repeat (ITR) sequence region and a 3′ ITR sequence region, three H1 promoter sequence regions, three modulatory polynucleotide regions targeting the same gene of interest (HTT), and three H1 terminator sequence regions, and three H1 terminator sequence regions, where each modulatory polynucleotide region is driven by its own H1 promoter and followed by its own H1 terminator.

In one embodiment, the polycistronic AAV particle viral genome comprises SEQ ID NO: 1847 (VOYPC31) which comprises a 5′ inverted terminal repeat (ITR) sequence region and a 3′ ITR sequence region, three H1 promoter sequence regions, three modulatory polynucleotide regions targeting the same gene of interest (HTT), and three H1 terminator sequence regions, where each modulatory polynucleotide region is driven by its own H1 promoter and followed by its own H1 terminator.

In one embodiment, the polycistronic AAV particle viral genome comprises SEQ ID NO: 1848 (VOYPC32) which comprises a 5′ inverted terminal repeat (ITR) sequence region and a 3′ ITR sequence region, three H1 promoter sequence regions, three modulatory polynucleotide regions targeting the same gene of interest (HTT), and three H1 terminator sequence regions, where each modulatory polynucleotide region is driven by its own H1 promoter and followed by its own H1 terminator.

In one embodiment, the polycistronic AAV particle viral genome comprises SEQ ID NO: 1849 (VOYPC33) which comprises a 5′ inverted terminal repeat (ITR) sequence region and a 3′ ITR sequence region, three H1 promoter sequence regions, three modulatory polynucleotide regions targeting the same gene of interest (HTT), and three H1 terminator sequence regions, where each modulatory polynucleotide region is driven by its own H1 promoter and followed by its own H1 terminator.

In one embodiment, the polycistronic AAV particle viral genome comprises SEQ ID NO: 1850 (VOYPC34) which comprises a 5′ inverted terminal repeat (ITR) sequence region and a 3′ ITR sequence region, three H1 promoter sequence regions, three modulatory polynucleotide regions targeting the same gene of interest (HTT), and three H1 terminator sequence regions, where each modulatory polynucleotide region is driven by its own H1 promoter and followed by its own H1 terminator.

In one embodiment, the polycistronic AAV particle viral genome comprises two promoter sequence regions, three modulatory polynucleotide regions and at least one polyadenylation sequence region.

In one embodiment, the polycistronic AAV particle viral genome comprises a CMV promoter sequence region, a T7 primer binding site, three modulatory polynucleotide sequence regions targeting the same gene of interest (HTT) and a polyadenylation sequence region. Non-limiting examples of sequences for use in the polycistronic AAV particles of the present invention having all of the sequence modules above are described in Table 37. In Table 37, the sequence identifier or sequence of the sequence region (Region SEQ ID NO) and the length of the sequence region (Region length) are described as well as the name of the sequence (e.g., VOYPC35).

TABLE 37
Sequence Regions

| | VOYPC35 | | VOYPC36 | |
|---|---|---|---|---|
| Sequence | Region | Region | Region | Region |
| Regions | SEQ ID NO | length | SEQ ID NO | length |
| CMV Promoter | 1817 | 588 | 1817 | 588 |
| T7 Primer | 1820 | 17 | 1820 | 17 |
| Binding Site | | | | |
| Modulatory | 1599 | 260 | 1599 | 260 |
| Polynucleotide | | | | |
| (VOYHTmiR-127.579) | | | | |
| Modulatory | 1589 | 158 | 1589 | 158 |
| Polynucleotide | | | | |
| (VOYHTmiR-104.016) | | | | |
| Modulatory | 1589 | 158 | — | — |
| Polynucleotide | | | | |
| (VOYHTmiR-104.016) | | | | |
| Modulatory | — | — | 1599 | 260 |
| Polynucleotide | | | | |
| (VOYHTmiR-127.579) | | | | |
| PolyA | 1828 | 225 | 1828 | 225 |

In one embodiment, the polycistronic AAV particle viral genome comprises the sequence modules described in VOYPC35 which comprises a CMV promoter sequence region, a T7 primer binding site, three modulatory polynucleotide regions targeting the same gene of interest (HTT), and a polyadenylation sequence region.

In one embodiment, the polycistronic AAV particle viral genome comprises three promoter sequence regions, and three modulatory polynucleotide regions.

In one embodiment, the polycistronic AAV particle viral genome comprises a GTTG region, two H1 promoter sequence regions, and three modulatory polynucleotide sequence regions targeting the same gene of interest (HTT). Non-limiting examples of sequences for use in the polycistronic AAV particles of the present invention having all of the sequence modules above are described in Tables 38 and 39. In Tables 38 and 39, the sequence identifier or sequence of the sequence region (Region SEQ ID NO) and the length of the sequence region (Region length) are described as well as the name of the sequence (e.g., VOYPC37).

TABLE 38
Sequence Regions

| | VOYPC37 | | VOYPC38 | | VOYPC41 | |
|---|---|---|---|---|---|---|
| Sequence | Region | Region | Region | Region | Region | Region |
| Regions | SEQ ID NO | length | SEQ ID NO | length | SEQ ID NO | length |
| GTTG | — | 4 | — | 4 | — | 4 |
| H1 Promoter | 1819 | 219 | 1819 | 219 | 1819 | 219 |
| Modulatory | 1599 | 260 | 1599 | 260 | 1599 | 260 |
| Polynucleotide | | | | | | |
| (VOYHTmiR-127.579) | | | | | | |
| H1 promoter | 1819 | 219 | 1819 | 219 | 1819 | 219 |
| Modulatory | 1599 | 260 | 1599 | 260 | — | — |
| Polynucleotide | | | | | | |
| (VOYHTmiR-127.579) | | | | | | |
| Modulatory | — | — | — | — | 1589 | 158 |
| Polynucleotide | | | | | | |
| (VOYHTmiR-104.016) | | | | | | |
| H1 promoter | 1819 | 219 | 1819 | 219 | 1819 | 219 |
| Modulatory | 1599 | 260 | — | — | 1599 | 260 |
| Polynucleotide | | | | | | |
| (VOYHTmiR-127.579) | | | | | | |
| Modulatory | — | — | 1589 | 158 | — | — |
| Polynucleotide | | | | | | |
| (VOYHTmiR-104.016) | | | | | | |

TABLE 39
Sequence Regions

| | VOYPC39 | | VOYPC40 | | VOYPC42 | |
|---|---|---|---|---|---|---|
| Sequence | Region | Region | Region | Region | Region | Region |
| Regions | SEQ ID NO | length | SEQ ID NO | length | SEQ ID NO | length |
| GTTG | — | 4 | — | 4 | — | 4 |
| H1 Promoter | 1819 | 219 | 1819 | 219 | 1819 | 219 |
| Modulatory | 1589 | 158 | 1589 | 158 | 1589 | 158 |
| Polynucleotide | | | | | | |
| (VOYHTmiR-104.016) | | | | | | |
| H1 promoter | 1819 | 219 | 1819 | 219 | 1819 | 219 |
| Modulatory | 1589 | 158 | 1589 | 158 | — | — |
| Polynucleotide | | | | | | |
| (VOYHTmiR-104.016) | | | | | | |
| Modulatory | — | — | — | — | 1599 | 260 |
| Polynucleotide | | | | | | |
| (VOYHTmiR-127.579) | | | | | | |
| H1 promoter | 1819 | 219 | 1819 | 219 | 1819 | 219 |
| Modulatory | — | — | 1599 | 260 | — | — |
| Polynucleotide | | | | | | |
| (VOYHTmiR-127.579) | | | | | | |
| Modulatory | 1589 | 158 | — | — | 1589 | 158 |
| Polynucleotide | | | | | | |
| (VOYHTmiR-104.016) | | | | | | |

In one embodiment, the polycistronic AAV particle viral genome comprises the sequence modules described in VOYPC37 which comprises a GTTG region, three H1 promoter sequence region, and three modulatory polynucleotide regions targeting the same gene of interest (HTT).

In one embodiment, the polycistronic AAV particle viral genome comprises the sequence modules described in VOYPC38 which comprises a GTTG region, three H1 promoter sequence region, and three modulatory polynucleotide regions targeting the same gene of interest (HTT).

In one embodiment, the polycistronic AAV particle viral genome comprises the sequence modules described in VOYPC39 which comprises a GTTG, three H1 promoter sequence region, and three modulatory polynucleotide regions targeting the same gene of interest (HTT).

In one embodiment, the polycistronic AAV particle viral genome comprises the sequence modules described in VOYPC40 which comprises a GTTG region, three H1 promoter sequence region, and three modulatory polynucleotide regions targeting the same gene of interest (HTT).

In one embodiment, the polycistronic AAV particle viral genome comprises the sequence modules described in VOYPC41 which comprises a GTTG region, three H1 promoter sequence region, and three modulatory polynucleotide regions targeting the same gene of interest (HTT).

In one embodiment, the polycistronic AAV particle viral genome comprises the sequence modules described in VOYPC42 which comprises a GTTG region, three H1 promoter sequence region, and three modulatory polynucleotide regions targeting the same gene of interest (HTT).

In one embodiment, the polycistronic AAV particle viral genome comprises at least one inverted terminal repeat (ITR) sequence region, at least one promoter sequence region, and four modulatory polynucleotide regions.

In one embodiment, the polycistronic AAV particle viral genome comprises a 5′ inverted terminal repeat (ITR) sequence region and a 3′ ITR sequence region, four H1 promoter sequence regions, four modulatory polynucleotide sequence regions targeting the same gene of interest (HTT), and four H1 terminator sequence regions, where each modulatory polynucleotide region is driven by its own H1 promoter and followed by its own H1 terminator.

Non-limiting examples of an ITR to ITR sequence for use in the polycistronic AAV particles of the present invention having all of the sequence modules above are described in Tables 40 and 41. In Tables 40 and 41, the sequence identifier or sequence of the sequence region (Region SEQ ID NO) and the length of the sequence region (Region length) are described as well as the name and sequence identifier of the ITR to ITR sequence (e.g., VOYPC43 (SEQ ID NO: 1851)).

TABLE 40
Sequence Regions in ITR to ITR Sequence

| | VOYPC43 (SEQ ID NO: 1851) | | VOYPC44 (SEQ ID NO: 1852) | | VOYPC45 (SEQ ID NO: 1853) | |
|---|---|---|---|---|---|---|
| Sequence | Region | Region | Region | Region | Region | Region |
| Regions | SEQ ID NO | length | SEQ ID NO | length | SEQ ID NO | length |
| 5′ ITR | 1788 | 105 | 1788 | 105 | 1788 | 105 |
| H1 promoter | 1819 | 219 | 1819 | 219 | 1819 | 219 |
| Modulatory | 1599 | 260 | 1599 | 260 | 1599 | 260 |
| Polynucleotide | | | | | | |
| (VOYHTmiR-127.579) | | | | | | |
| H1 Terminator | 2681 | 5 | 2681 | 5 | 2681 | 5 |
| H1 promoter | 1819 | 219 | 1819 | 219 | 1819 | 219 |
| Modulatory | 1599 | 260 | — | — | — | — |
| Polynucleotide | | | | | | |
| (VOYHTmiR-127.579) | | | | | | |
| Modulatory | — | — | 1589 | 158 | 1589 | 158 |
| Polynucleotide | | | | | | |
| (VOYHTmiR-104.016) | | | | | | |
| H1 Terminator | 2681 | 5 | 2681 | 5 | 2681 | 5 |
| H1 promoter | 1819 | 219 | 1819 | 219 | 1819 | 219 |
| Modulatory | 1599 | 260 | 1599 | 260 | — | — |
| Polynucleotide | | | | | | |
| (VOYHTmiR-127.579) | | | | | | |
| Modulatory | — | — | — | — | 1589 | 158 |
| Polynucleotide | | | | | | |
| (VOYHTmiR-104.016) | | | | | | |
| H1 Terminator | 2681 | 5 | 2681 | 5 | 2681 | 5 |
| H1 promoter | 1819 | 219 | 1819 | 219 | 1819 | 219 |
| Modulatory | 1599 | 260 | — | — | 1599 | 260 |
| Polynucleotide | | | | | | |
| (VOYHTmiR-127.579) | | | | | | |
| Modulatory | — | — | 1589 | 158 | — | — |
| Polynucleotide | | | | | | |
| (VOYHTmiR-104.016) | | | | | | |
| H1 Terminator | 2681 | 5 | 2681 | 5 | 2681 | 5 |
| 3′ ITR | 1790 | 130 | 1790 | 130 | 1790 | 130 |

TABLE 41
Sequence Regions in ITR to ITR Sequence

| | VOYPC46 (SEQ ID NO: 1854) | | VOYPC47 (SEQ ID NO: 1855) | | VOYPC48 (SEQ ID NO: 1856) | |
|---|---|---|---|---|---|---|
| Sequence | Region | Region | Region | Region | Region | Region |
| Regions | SEQ ID NO | length | SEQ ID NO | length | SEQ ID NO | length |
| 5′ ITR | 1788 | 105 | 1788 | 105 | 1788 | 105 |
| H1 promoter | 1819 | 219 | 1819 | 219 | 1819 | 219 |
| Modulatory | 1589 | 158 | 1589 | 158 | 1589 | 158 |
| Polynucleotide | | | | | | |
| (VOYHTmiR-104.016) | | | | | | |
| H1 Terminator | 2681 | 5 | 2681 | 5 | 2681 | 5 |
| H1 promoter | 1819 | 219 | 1819 | 219 | 1819 | 219 |
| Modulatory | 1599 | 260 | — | — | 1599 | 260 |
| Polynucleotide | | | | | | |
| (VOYHTmiR-127.579) | | | | | | |
| Modulatory | — | — | 1589 | 158 | — | — |
| Polynucleotide | | | | | | |
| (VOYHTmiR-104.016) | | | | | | |
| H1 Terminator | 2681 | 5 | 2681 | 5 | 2681 | 5 |
| H1 promoter | 1819 | 219 | 1819 | 219 | 1819 | 219 |
| Modulatory | 1599 | 260 | — | — | — | — |
| Polynucleotide | | | | | | |
| (VOYHTmiR-127.579) | | | | | | |
| Modulatory | — | — | 1589 | 158 | 1589 | 158 |
| Polynucleotide | | | | | | |
| (VOYHTmiR-104.016) | | | | | | |
| H1 Terminator | 2681 | 5 | 2681 | 5 | 2681 | 5 |
| H1 promoter | 1819 | 219 | 1819 | 219 | 1819 | 219 |
| Modulatory | — | — | — | — | 1599 | 260 |
| Polynucleotide | | | | | | |
| (VOYHTmiR-127.579) | | | | | | |
| Modulatory | 1589 | 158 | 1589 | 158 | — | — |
| Polynucleotide | | | | | | |
| (VOYHTmiR-104.016) | | | | | | |
| H1 Terminator | 2681 | 5 | 2681 | 5 | 2681 | 5 |
| 3′ ITR | 1790 | 130 | 1790 | 130 | 1790 | 130 |

In one embodiment, the polycistronic AAV particle viral genome comprises SEQ ID NO: 1851 (VOYPC43) which comprises a 5′ inverted terminal repeat (ITR) sequence region and a 3′ ITR sequence region, four H1 promoter sequence regions, four modulatory polynucleotide regions targeting the same gene of interest (HTT), and four H1 terminator sequence regions, where each modulatory polynucleotide region is driven by its own H1 promoter and followed by its own H1 terminator.

In one embodiment, the polycistronic AAV particle viral genome comprises SEQ ID NO: 1852 (VOYPC44) which comprises a 5′ inverted terminal repeat (ITR) sequence region and a 3′ ITR sequence region, four H1 promoter sequence regions, four modulatory polynucleotide regions targeting the same gene of interest (HTT), and four H1 terminator sequence regions, where each modulatory polynucleotide region is driven by its own H1 promoter and followed by its own H1 terminator.

In one embodiment, the polycistronic AAV particle viral genome comprises SEQ ID NO: 1853 (VOYPC45) which comprises a 5′ inverted terminal repeat (ITR) sequence region and a 3′ ITR sequence region, four H1 promoter sequence regions, four modulatory polynucleotide regions targeting the same gene of interest (HTT), and four H1 terminator sequence regions, where each modulatory polynucleotide region is driven by its own H1 promoter and followed by its own H1 terminator.

In one embodiment, the polycistronic AAV particle viral genome comprises SEQ ID NO: 1854 (VOYPC46) which comprises a 5′ inverted terminal repeat (ITR) sequence region and a 3′ ITR sequence region, four H1 promoter sequence regions, four modulatory polynucleotide regions targeting the same gene of interest (HTT), and four H1 terminator sequence regions, where each modulatory polynucleotide region is driven by its own H1 promoter and followed by its own H1 terminator.

In one embodiment, the polycistronic AAV particle viral genome comprises SEQ ID NO: 1855 (VOYPC47) which comprises a 5′ inverted terminal repeat (ITR) sequence region and a 3′ ITR sequence region, four H1 promoter sequence regions, four modulatory polynucleotide regions targeting the same gene of interest (HTT), and four H1 terminator sequence regions, where each modulatory polynucleotide region is driven by its own H1 promoter and followed by its own H1 terminator.

In one embodiment, the polycistronic AAV particle viral genome comprises SEQ ID NO: 1856 (VOYPC48) which comprises a 5′ inverted terminal repeat (ITR) sequence region and a 3′ ITR sequence region, four H1 promoter sequence regions, four modulatory polynucleotide regions targeting the same gene of interest (HTT), and four H1 terminator sequence regions, where each modulatory polynucleotide region is driven by its own H1 promoter and followed by its own H1 terminator.

In one embodiment, the polycistronic AAV particle viral genome comprises at least one inverted terminal repeat (ITR) sequence region, at least one enhancer sequence region, at least one intron sequence region, at least one promoter sequence region, and four modulatory polynucleotide regions.

In one embodiment, the polycistronic AAV particle viral genome comprises a 5′ inverted terminal repeat (ITR) sequence region and a 3′ ITR sequence region, a CMV enhancer sequence region, four H1 promoter sequence regions, and four modulatory polynucleotide sequence regions targeting the same gene of interest (HTT). Non-limiting examples of an ITR to ITR sequence for use in the polycistronic AAV particles of the present invention having all of the sequence modules above are described in Table 42. In Table 42, the sequence identifier or sequence of the sequence region (Region SEQ ID NO) and the length of the sequence region (Region length) are described as well as the name and sequence identifier of the ITR to ITR sequence (e.g., VOYPC49 (SEQ ID NO: 1857)).

TABLE 42
Sequence Regions in ITR to ITR Sequence

| | VOYPC49 | | VOYPC50 | |
|---|---|---|---|---|
| | (SEQ ID NO: 1857) | | (SEQ ID NO: 1858) | |
| Sequence | Region | Region | Region | Region |
| Regions | SEQ ID NO | length | SEQ ID NO | length |
| 5′ ITR | 1788 | 105 | 1788 | 105 |
| CMV enhancer | 1814 | 382 | 1814 | 382 |
| CBA Promoter | 1816 | 260 | 1816 | 260 |
| SV40 intron | 1826 | 201 | 1826 | 201 |
| Modulatory | 1599 | 260 | 1599 | 260 |
| Polynucleotide | | | | |
| (VOYHTmiR-127.579) | | | | |
| Modulatory | 1589 | 158 | 1589 | 158 |
| Polynucleotide | | | | |
| (VOYHTmiR-104.016) | | | | |
| Modulatory | 1599 | 260 | — | — |
| Polynucleotide | | | | |
| (VOYHTmiR-127.579) | | | | |
| Modulatory | — | — | 1589 | 158 |
| Polynucleotide | | | | |
| (VOYHTmiR-104.016) | | | | |
| Modulatory | — | — | 1599 | 260 |
| Polynucleotide | | | | |
| (VOYHTmiR-127.579) | | | | |
| Modulatory | 1589 | 158 | — | — |
| Polynucleotide | | | | |
| (VOYHTmiR-104.016) | | | | |
| PolyA | 1827 | 127 | 1827 | 127 |
| 3′ ITR | 1790 | 130 | 1790 | 130 |

In one embodiment, the polycistronic AAV particle viral genome comprises SEQ ID NO: 1857 (VOYPC49) which comprises a 5′ inverted terminal repeat (ITR) sequence region and a 3′ ITR sequence region, a CMV enhancer, a SV40 intron, four modulatory polynucleotide regions targeting the same gene of interest (HTT), and a polyadenylation sequence region.

In one embodiment, the polycistronic AAV particle viral genome comprises SEQ ID NO: 1858 (VOYPC50) which comprises a 5′ inverted terminal repeat (ITR) sequence region and a 3′ ITR sequence region, a CMV enhancer, a SV40 intron, four modulatory polynucleotide regions targeting the same gene of interest (HTT), and a polyadenylation sequence region.

In one embodiment, the polycistronic AAV particle viral genome comprises two promoter sequence regions, four modulatory polynucleotide regions and at least one polyadenylation sequence region.

In one embodiment, the polycistronic AAV particle viral genome comprises a CMV promoter sequence region, a T7 primer binding site region, four modulatory polynucleotide sequence regions targeting the same gene of interest (HTT) and a polyadenylation sequence region. Non-limiting examples of sequences for use in the polycistronic AAV particles of the present invention having all of the sequence modules above are described in Table 43. In Table 43, the sequence identifier or sequence of the sequence region (Region SEQ ID NO) and the length of the sequence region (Region length) are described as well as the name of the sequence (e.g., VOYPC51).

TABLE 43
Sequence Regions

| | VOYPC51 | | VOYPC52 | |
|---|---|---|---|---|
| Sequence | Region | Region | Region | Region |
| Regions | SEQ ID NO | length | SEQ ID NO | length |
| CMV Promoter | 1817 | 588 | 1817 | 588 |
| T7 Primer | 1820 | 17 | 1820 | 17 |
| binding site | | | | |
| Modulatory | 1599 | 260 | 1599 | 260 |
| Polynucleotide | | | | |
| (VOYHTmiR-127.579) | | | | |
| Modulatory | 1589 | 158 | 1589 | 158 |
| Polynucleotide | | | | |
| (VOYHTmiR-104.016) | | | | |
| Modulatory | — | — | 1589 | 158 |
| Polynucleotide | | | | |
| (VOYHTmiR-104.016) | | | | |
| Modulatory | 1599 | 260 | — | — |
| Polynucleotide | | | | |
| (VOYHTmiR-127.579) | | | | |
| Modulatory | 1589 | 158 | — | — |
| Polynucleotide | | | | |
| (VOYHTmiR-104.016) | | | | |
| Modulatory | — | — | 1599 | 260 |
| Polynucleotide | | | | |
| (VOYHTmiR-127.579) | | | | |
| PolyA | 1828 | 225 | 1828 | 225 |

In one embodiment, the polycistronic AAV particle viral genome comprises the sequence modules described in VOYPC51 which comprises a CMV promoter sequence region, a T7 primer binding site region, four modulatory polynucleotide regions targeting the same gene of interest (HTT), and a polyadenylation sequence region.

In one embodiment, the polycistronic AAV particle viral genome comprises the sequence modules described in VOYPC52 which comprises a CMV promoter sequence region, a T7 primer binding site region, four modulatory polynucleotide regions targeting the same gene of interest (HTT), and a polyadenylation sequence region.

In one embodiment, the polycistronic AAV particle viral genome comprises five promoter sequence regions and four modulatory polynucleotide regions.

In one embodiment, the polycistronic AAV particle viral genome comprises a GTTG region, four H1 promoter sequence regions, and four modulatory polynucleotide sequence regions targeting the same gene of interest (HTT). Non-limiting examples of sequences for use in the polycistronic AAV particles of the present invention having all of the sequence modules above are described in Tables 44 and 45. In Tables 44 and 45, the sequence identifier or sequence of the sequence region (Region SEQ ID NO) and the length of the sequence region (Region length) are described as well as the name of the sequence (e.g., VOYPC53).

TABLE 44
Sequence Regions

| | VOYPC53 | | VOYPC54 | | VOYPC55 | |
|---|---|---|---|---|---|---|
| Sequence | Region | Region | Region | Region | Region | Region |
| Regions | SEQ ID NO | length | SEQ ID NO | length | SEQ ID NO | length |
| GTTG | — | 4 | — | 4 | — | 4 |
| H1 Promoter | 1819 | 219 | 1819 | 219 | 1819 | 219 |
| Modulatory | 1599 | 260 | 1599 | 260 | 1599 | 260 |
| Polynucleotide | | | | | | |
| (VOYHTmiR-127.579) | | | | | | |
| H1 promoter | 1819 | 219 | 1819 | 219 | 1819 | 219 |
| Modulatory | 1599 | 260 | — | — | — | — |
| Polynucleotide | | | | | | |
| (VOYHTmiR-127.579) | | | | | | |
| Modulatory | — | — | 1589 | 158 | 1589 | 158 |
| Polynucleotide | | | | | | |
| (VOYHTmiR-104.016) | | | | | | |
| H1 promoter | 1819 | 219 | 1819 | 219 | 1819 | 219 |
| Modulatory | 1599 | 260 | 1599 | 260 | — | — |
| Polynucleotide | | | | | | |
| (VOYHTmiR-127.579) | | | | | | |
| Modulatory | — | — | — | — | 1589 | 158 |
| Polynucleotide | | | | | | |
| (VOYHTmiR-104.016) | | | | | | |
| Hl promoter | 1819 | 219 | 1819 | 219 | 1819 | 219 |
| Modulatory | 1599 | 260 | — | — | 1599 | 260 |
| Polynucleotide | | | | | | |
| (VOYHTmiR-127.579) | | | | | | |
| Modulatory | — | — | 1589 | 158 | — | — |
| Polynucleotide | | | | | | |
| (VOYHTmiR-104.016) | | | | | | |

TABLE 45
Sequence Regions

| | VOYPC56 | | VOYPC57 | | VOYPC58 | |
|---|---|---|---|---|---|---|
| Sequence | Region | Region | Region | Region | Region | Region |
| Regions | SEQ ID NO | length | SEQ ID NO | length | SEQ ID NO | length |
| GTTG | — | 4 | — | 4 | — | 4 |
| H1 Promoter | 1819 | 219 | 1819 | 219 | 1819 | 219 |
| Modulatory | 1589 | 158 | 1589 | 158 | 1589 | 158 |
| Polynucleotide | | | | | | |
| (VOYHTmiR-104.016) | | | | | | |
| H1 promoter | 1819 | 219 | 1819 | 219 | 1819 | 219 |
| Modulatory | 1599 | 260 | 1599 | 260 | — | — |
| Polynucleotide | | | | | | |
| (VOYHTmiR-127.579) | | | | | | |
| Modulatory | — | — | — | — | 1589 | 158 |
| Polynucleotide | | | | | | |
| (VOYHTmiR-104.016) | | | | | | |
| H1 promoter | 1819 | 219 | 1819 | 219 | 1819 | 219 |
| Modulatory | — | — | 1599 | 260 | — | — |
| Polynucleotide | | | | | | |
| (VOYHTmiR-127.579) | | | | | | |
| Modulatory | 1589 | 158 | — | — | 1589 | 158 |
| Polynucleotide | | | | | | |
| (VOYHTmiR-104.016) | | | | | | |
| H1 promoter | 1819 | 219 | 1819 | 219 | 1819 | 219 |
| Modulatory | 1599 | 260 | — | — | — | — |
| Polynucleotide | | | | | | |
| (VOYHTmiR-127.579) | | | | | | |
| Modulatory | — | — | 1589 | 158 | 1589 | 158 |
| Polynucleotide | | | | | | |
| (VOYHTmiR-104.016) | | | | | | |

In one embodiment, the polycistronic AAV particle viral genome comprises the sequence modules described in VOYPC53 which comprises a GTTG region, four H1 promoter sequence regions, and four modulatory polynucleotide regions targeting the same gene of interest (HTT).

In one embodiment, the polycistronic AAV particle viral genome comprises the sequence modules described in VOYPC54 which comprises a GTTG region, four H1 promoter sequence regions, and four modulatory polynucleotide regions targeting the same gene of interest (HTT).

In one embodiment, the polycistronic AAV particle viral genome comprises the sequence modules described in VOYPC55 which comprises a GTTG region, four H1 promoter sequence regions, and four modulatory polynucleotide regions targeting the same gene of interest (HTT).

In one embodiment, the polycistronic AAV particle viral genome comprises the sequence modules described in VOYPC56 which comprises a GTTG region, four H1 promoter sequence regions, and four modulatory polynucleotide regions targeting the same gene of interest (HTT).

In one embodiment, the polycistronic AAV particle viral genome comprises the sequence modules described in VOYPC57 which comprises a GTTG region, four H1 promoter sequence regions, and four modulatory polynucleotide regions targeting the same gene of interest (HTT).

In one embodiment, the polycistronic AAV particle viral genome comprises the sequence modules described in VOYPC58 which comprises a GTTG region, four H1 promoter sequence regions, and four modulatory polynucleotide regions targeting the same gene of interest (HTT).

### Viral Production

The present disclosure provides a method for the generation of parvoviral particles, e.g. AAV particles, by viral genome replication in a viral replication cell comprising contacting the viral replication cell with an AAV polynucleotide or AAV genome.

The present disclosure provides a method for producing an AAV particle having enhanced (increased, improved) transduction efficiency comprising the steps of: 1) co-transfecting competent bacterial cells with a bacmid vector and either a viral construct vector and/or AAV payload construct vector, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, 5) harvesting and purifying the viral particle comprising a parvoviral genome.

In one embodiment, the present invention provides a method for producing an AAV particle comprising the steps of 1) simultaneously co-transfecting mammalian cells, such as, but not limited to HEK293 cells, with a payload region, a construct expressing rep and cap genes and a helper construct, 2) harvesting and purifying the AAV particle comprising a viral genome.

### Cells

The present disclosure provides a cell comprising an AAV polynucleotide and/or AAV genome.

Viral production disclosed herein describes processes and methods for producing AAV particles that contact a target cell to deliver a payload construct, e.g. a recombinant viral construct, which comprises a polynucleotide sequence encoding a payload molecule.

In one embodiment, the AAV particles may be produced in a viral replication cell that comprises an insect cell.

Growing conditions for insect cells in culture, and production of heterologous products in insect cells in culture are well-known in the art, see U.S. Pat. No. 6,204,059, the contents of which are herein incorporated by reference in their entirety.

Any insect cell which allows for replication of parvovirus and which can be maintained in culture can be used in accordance with the present invention. Cell lines may be used from Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, N J (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which is herein incorporated by reference in its entirety.

The viral replication cell may be selected from any biological organism, including prokaryotic (e.g., bacterial) cells, and eukaryotic cells, including, insect cells, yeast cells and mammalian cells. Viral replication cells may comprise mammalian cells such as A549, WEH1, 3T3, 10T1/2, BHK, MDCK, COS 1, COS 7, BSC 1, BSC 40, BMT 10, VERO. W138, HeLa, HEK293, Saos, C2C12, L cells, HT1080, HepG2 and primary fibroblast, hepatocyte and myoblast cells derived from mammals. Viral replication cells comprise cells derived from mammalian species including, but not limited to, human, monkey, mouse, rat, rabbit, and hamster or cell type, including but not limited to fibroblast, hepatocyte, tumor cell, cell line transformed cell, etc.

### Small Scale Production of AAV Particles

Viral production disclosed herein describes processes and methods for producing AAV particles that contact a target cell to deliver a payload, e.g. a recombinant viral construct, which comprises a polynucleotide sequence encoding a payload.

In one embodiment, the AAV particles may be produced in a viral replication cell that comprises a mammalian cell.

Viral replication cells commonly used for production of recombinant AAV particles include, but are not limited to 293 cells, COS cells, HeLa cells, KB cells, and other mammalian cell lines as described in U.S. Pat. Nos. 6,156,303, 5,387,484, 5,741,683, 5,691,176, and 5,688,676; U.S. patent application 2002/0081721, and International Patent Applications WO 00/47757, WO 00/24916, and WO 96/17947, the contents of each of which are herein incorporated by reference in their entireties.

In one embodiment, AAV particles are produced in mammalian-cells wherein all three VP proteins are expressed at a stoichiometry approaching 1:1:10 (VP1:VP2:VP3). The regulatory mechanisms that allow this controlled level of expression include the production of two mRNAs, one for VP1, and the other for VP2 and VP3, produced by differential splicing.

In another embodiment, AAV particles are produced in mammalian cells using a triple transfection method wherein a payload construct, parvoviral Rep and parvoviral Cap and a helper construct are comprised within three different constructs. The triple transfection method of the three components of AAV particle production may be utilized to produce small lots of virus for assays including transduction efficiency, target tissue (tropism) evaluation, and stability.

### Baculovirus

Particle production disclosed herein describes processes and methods for producing AAV particles that contact a target cell to deliver a payload construct which comprises a polynucleotide sequence encoding a payload.

Briefly, the viral construct vector and the AAV payload construct vector are each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector. The two baculoviruses may be used to infect a single viral replication cell population for production of AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al., J Virol. 2006 February; 80 (4):1874-85, the contents of which are herein incorporated by reference in their entirety.

Production of AAV particles with baculovirus in an insect cell system may address known baculovirus genetic and physical instability. In one embodiment, the production system addresses baculovirus instability over multiple passages by utilizing a titerless infected-cells preservation and scale-up system. Small scale seed cultures of viral producing cells are transfected with viral expression constructs encoding the structural, non-structural, components of the viral particle. Baculovirus-infected viral producing cells are harvested into aliquots that may be cryopreserved in liquid nitrogen; the aliquots retain viability and infectivity for infection of large scale viral producing cell culture Wasilko D J et al., Protein Expr Purif. 2009 June; 65(2):122-32, the contents of which are herein incorporated by reference in their entirety.

A genetically stable baculovirus may be used to produce source of the one or more of the components for producing AAV particles in invertebrate cells. In one embodiment, defective baculovirus expression vectors may be maintained episomally in insect cells. In such an embodiment the bacmid vector is engineered with replication control elements, including but not limited to promoters, enhancers, and/or cell-cycle regulated replication elements.

In one embodiment, baculoviruses may be engineered with a (non-) selectable marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In one embodiment, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

### Large-Scale Production

In some embodiments, AAV particle production may be modified to increase the scale of production. Large scale viral production methods according to the present disclosure may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Methods of increasing viral particle production scale typically comprise increasing the number of viral replication cells. In some embodiments, viral replication cells comprise adherent cells. To increase the scale of viral particle production by adherent viral replication cells, larger cell culture surfaces are required. In some cases, large-scale production methods comprise the use of roller bottles to increase cell culture surfaces. Other cell culture substrates with increased surface areas are known in the art. Examples of additional adherent cell culture products with increased surface areas include, but are not limited to CELLSTACK®, CELLCUBE® (Corning Corp., Corning, N.Y.) and NUNC™ CELL FACTORY™ (Thermo Scientific, Waltham, Mass.) In some cases, large-scale adherent cell surfaces may comprise from about 1,000 cm² to about 100,000 cm². In some cases, large-scale adherent cell cultures may comprise from about 10⁷ to about 10⁹ cells, from about 10⁸ to about 10¹⁰ cells, from about 10⁹ to about 10¹² cells or at least 10¹² cells. In some cases, large-scale adherent cultures may produce from about 10⁹ to about 10¹², from about 10¹⁰ to about 10¹³, from about 10¹¹ to about 10¹⁴, from about 10¹² to about 10¹⁵ or at least 10¹⁵ viral particles.

In some embodiments, large-scale viral production methods of the present disclosure may comprise the use of suspension cell cultures. Suspension cell culture allows for significantly increased numbers of cells. Typically, the number of adherent cells that can be grown on about 10-50 cm² of surface area can be grown in about 1 cm³ volume in suspension.

Transfection of replication cells in large-scale culture formats may be carried out according to any methods known in the art. For large-scale adherent cell cultures, transfection methods may include, but are not limited to the use of inorganic compounds (e.g. calcium phosphate), organic compounds [e.g. polyethyleneimine (PEI)] or the use of non-chemical methods (e.g. electroporation.) With cells grown in suspension, transfection methods may include, but are not limited to the use of calcium phosphate and the use of PEI. In some cases, transfection of large scale suspension cultures may be carried out according to the section entitled “Transfection Procedure” described in Feng, L. et al., 2008. Biotechnol Appl. Biochem. 50:121-32, the contents of which are herein incorporated by reference in their entirety. According to such embodiments, PEI-DNA complexes may be formed for introduction of plasmids to be transfected. In some cases, cells being transfected with PEI-DNA complexes may be ‘shocked’ prior to transfection. This comprises lowering cell culture temperatures to 4° C. for a period of about 1 hour. In some cases, cell cultures may be shocked for a period of from about 10 minutes to about 5 hours. In some cases, cell cultures may be shocked at a temperature of from about 0° C. to about 20° C.

In some cases, transfections may include one or more vectors for expression of an RNA effector molecule to reduce expression of nucleic acids from one or more AAV payload construct. Such methods may enhance the production of viral particles by reducing cellular resources wasted on expressing payload constructs. In some cases, such methods may be carried according to those taught in US Publication No. US2014/0099666, the contents of which are herein incorporated by reference in their entirety.

### Bioreactors

In some embodiments, cell culture bioreactors may be used for large scale viral production. In some cases, bioreactors comprise stirred tank reactors. Such reactors generally comprise a vessel, typically cylindrical in shape, with a stirrer (e.g. impeller.) In some embodiments, such bioreactor vessels may be placed within a water jacket to control vessel temperature and/or to minimize effects from ambient temperature changes. Bioreactor vessel volume may range in size from about 500 ml to about 2 L, from about 1 L to about 5 L, from about 2.5 L to about 20 L, from about 10 L to about 50 L, from about 25 L to about 100 L, from about 75 L to about 500 L, from about 250 L to about 2,000 L, from about 1,000 L to about 10,000 L, from about 5,000 L to about 50,000 L or at least 50,000 L. Vessel bottoms may be rounded or flat. In some cases, animal cell cultures may be maintained in bioreactors with rounded vessel bottoms.

In some cases, bioreactor vessels may be warmed through the use of a thermocirculator. Thermocirculators pump heated water around water jackets. In some cases, heated water may be pumped through pipes (e.g. coiled pipes) that are present within bioreactor vessels. In some cases, warm air may be circulated around bioreactors, including, but not limited to air space directly above culture medium. Additionally, pH and CO₂ levels may be maintained to optimize cell viability.

In some cases, bioreactors may comprise hollow-fiber reactors. Hollow-fiber bioreactors may support the culture of both anchorage dependent and anchorage independent cells. Further bioreactors may include, but are not limited to packed-bed or fixed-bed bioreactors. Such bioreactors may comprise vessels with glass beads for adherent cell attachment. Further packed-bed reactors may comprise ceramic beads.

In some cases, viral particles are produced through the use of a disposable bioreactor. In some embodiments, such bioreactors may include WAVE™ disposable bioreactors.

In some embodiments, AAV particle production in animal cell bioreactor cultures may be carried out according to the methods taught in U.S. Pat. Nos. 5,064,764, 6,194,191, 6,566,118, 8,137,948 or US Patent Application No. US2011/0229971, the contents of each of which are herein incorporated by reference in their entirety.

### Cell Lysis

Cells of the invention, including, but not limited to viral production cells, may be subjected to cell lysis according to any methods known in the art. Cell lysis may be carried out to obtain one or more agents (e.g. viral particles) present within any cells of the invention. In some embodiments, cell lysis may be carried out according to any of the methods listed in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Cell lysis methods may be chemical or mechanical. Chemical cell lysis typically comprises contacting one or more cells with one or more lysis agent. Mechanical lysis typically comprises subjecting one or more cells to one or more lysis condition and/or one or more lysis force.

In some embodiments, chemical lysis may be used to lyse cells. As used herein, the term “lysis agent” refers to any agent that may aid in the disruption of a cell. In some cases, lysis agents are introduced in solutions, termed lysis solutions or lysis buffers. As used herein, the term “lysis solution” refers to a solution (typically aqueous) comprising one or more lysis agent. In addition to lysis agents, lysis solutions may include one or more buffering agents, solubilizing agents, surfactants, preservatives, cryoprotectants, enzymes, enzyme inhibitors and/or chelators. Lysis buffers are lysis solutions comprising one or more buffering agent. Additional components of lysis solutions may include one or more solubilizing agent. As used herein, the term “solubilizing agent” refers to a compound that enhances the solubility of one or more components of a solution and/or the solubility of one or more entities to which solutions are applied. In some cases, solubilizing agents enhance protein solubility. In some cases, solubilizing agents are selected based on their ability to enhance protein solubility while maintaining protein conformation and/or activity.

Exemplary lysis agents may include any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In some cases, lysis agents may be selected from lysis salts, amphoteric agents, cationic agents, ionic detergents and non-ionic detergents. Lysis salts may include, but are not limited to sodium chloride (NaCl) and potassium chloride (KCl.) Further lysis salts may include any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety. Concentrations of salts may be increased or decreased to obtain an effective concentration for rupture of cell membranes. Amphoteric agents, as referred to herein, are compounds capable of reacting as an acid or a base. Amphoteric agents may include, but are not limited to lysophosphatidylcholine, 3-((3-Cholamidopropyl) dimethylammonium)-1-propanesulfonate (CHAPS), ZWITTERGENT® and the like. Cationic agents may include, but are not limited to cetyltrimethylammonium bromide (C (16) TAB) and Benzalkonium chloride. Lysis agents comprising detergents may include ionic detergents or non-ionic detergents. Detergents may function to break apart or dissolve cell structures including, but not limited to cell membranes, cell walls, lipids, carbohydrates, lipoproteins and glycoproteins. Exemplary ionic detergents include any of those taught in U.S. Pat. Nos. 7,625,570 and 6,593,123 or US Publication No. US2014/0087361, the contents of each of which are herein incorporated by reference in their entirety. Some ionic detergents may include, but are not limited to sodium dodecyl sulfate (SDS), cholate and deoxycholate. In some cases, ionic detergents may be included in lysis solutions as a solubilizing agent. Non-ionic detergents may include, but are not limited to octylglucoside, digitonin, lubrol, C12E8, TWEEN®-20, TWEEN®-80, Triton X-100 and Noniodet P-40. Non-ionic detergents are typically weaker lysis agents, but may be included as solubilizing agents for solubilizing cellular and/or viral proteins. Further lysis agents may include enzymes and urea. In some cases, one or more lysis agents may be combined in a lysis solution in order to enhance one or more of cell lysis and protein solubility. In some cases, enzyme inhibitors may be included in lysis solutions in order to prevent proteolysis that may be triggered by cell membrane disruption.

In some embodiments, mechanical cell lysis is carried out. Mechanical cell lysis methods may include the use of one or more lysis condition and/or one or more lysis force. As used herein, the term “lysis condition” refers to a state or circumstance that promotes cellular disruption. Lysis conditions may comprise certain temperatures, pressures, osmotic purity, salinity and the like. In some cases, lysis conditions comprise increased or decreased temperatures. According to some embodiments, lysis conditions comprise changes in temperature to promote cellular disruption. Cell lysis carried out according to such embodiments may include freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according freeze-thaw lysis methods, may further comprise one or more lysis agents, solubilizing agents, buffering agents, cryoprotectants, surfactants, preservatives, enzymes, enzyme inhibitors and/or chelators. Once cell solutions subjected to freezing are thawed, such components may enhance the recovery of desired cellular products. In some cases, one or more cryoprotectants are included in cell solutions undergoing freeze-thaw lysis. As used herein, the term “cryoprotectant” refers to an agent used to protect one or more substance from damage due to freezing. Cryoprotectants may include any of those taught in US Publication No. US2013/0323302 or U.S. Pat. Nos. 6,503,888, 6,180,613, 7,888,096, 7,091,030, the contents of each of which are herein incorporated by reference in their entirety. In some cases, cryoprotectants may include, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In some embodiments, freeze-thaw lysis may be carried out according to any of the methods described in U.S. Pat. No. 7,704,721, the contents of which are herein incorporated by reference in their entirety.

As used herein, the term “lysis force” refers to a physical activity used to disrupt a cell. Lysis forces may include, but are not limited to mechanical forces, sonic forces, gravitational forces, optical forces, electrical forces and the like. Cell lysis carried out by mechanical force is referred to herein as “mechanical lysis.” Mechanical forces that may be used according to mechanical lysis may include high shear fluid forces. According to such methods of mechanical lysis, a microfluidizer may be used. Microfluidizers typically comprise an inlet reservoir where cell solutions may be applied. Cell solutions may then be pumped into an interaction chamber via a pump (e.g. high-pressure pump) at high speed and/or pressure to produce shear fluid forces. Resulting lysates may then be collected in one or more output reservoir. Pump speed and/or pressure may be adjusted to modulate cell lysis and enhance recovery of products (e.g. viral particles.) Other mechanical lysis methods may include physical disruption of cells by scraping.

Cell lysis methods may be selected based on the cell culture format of cells to be lysed. For example, with adherent cell cultures, some chemical and mechanical lysis methods may be used. Such mechanical lysis methods may include freeze-thaw lysis or scraping. In another example, chemical lysis of adherent cell cultures may be carried out through incubation with lysis solutions comprising surfactant, such as Triton-X-100. In some cases, cell lysates generated from adherent cell cultures may be treated with one more nuclease to lower the viscosity of the lysates caused by liberated DNA.

In one embodiment, a method for harvesting AAV particles without lysis may be used for efficient and scalable AAV particle production. In a non-limiting example, AAV particles may be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107, the contents of which are incorporated herein by reference in their entirety.

### Clarification

Cell lysates comprising viral particles may be subjected to clarification. Clarification refers to initial steps taken in purification of viral particles from cell lysates. Clarification serves to prepare lysates for further purification by removing larger, insoluble debris. Clarification steps may include, but are not limited to centrifugation and filtration. During clarification, centrifugation may be carried out at low speeds to remove larger debris only. Similarly, filtration may be carried out using filters with larger pore sizes so that only larger debris is removed. In some cases, tangential flow filtration may be used during clarification. Objectives of viral clarification include high throughput processing of cell lysates and to optimize ultimate viral recovery. Advantages of including a clarification step include scalability for processing of larger volumes of lysate. In some embodiments, clarification may be carried out according to any of the methods presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

Methods of cell lysate clarification by filtration are well understood in the art and may be carried out according to a variety of available methods including, but not limited to passive filtration and flow filtration. Filters used may comprise a variety of materials and pore sizes. For example, cell lysate filters may comprise pore sizes of from about 1 μM to about 5 from about 0.5 μM to about 2 from about 0.1 μM to about 1 from about 0.05 μM to about 0.05 μM and from about 0.001 μM to about 0.1 Exemplary pore sizes for cell lysate filters may include, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 In one embodiment, clarification may comprise filtration through a filter with 2.0 μM pore size to remove large debris, followed by passage through a filter with 0.45 μM pore size to remove intact cells.

Filter materials may be composed of a variety of materials. Such materials may include, but are not limited to polymeric materials and metal materials (e.g. sintered metal and pored aluminum.) Exemplary materials may include, but are not limited to nylon, cellulose materials (e.g. cellulose acetate), polyvinylidene fluoride (PVDF), polyethersulfone, polyamide, polysulfone, polypropylene, and polyethylene terephthalate. In some cases, filters useful for clarification of cell lysates may include, but are not limited to ULTIPLEAT PROFILE™ filters (Pall Corporation, Port Washington, N.Y.), SUPOR™ membrane filters (Pall Corporation, Port Washington, N.Y.)

In some cases, flow filtration may be carried out to increase filtration speed and/or effectiveness. In some cases, flow filtration may comprise vacuum filtration. According to such methods, a vacuum is created on the side of the filter opposite that of cell lysate to be filtered. In some cases, cell lysates may be passed through filters by centrifugal forces. In some cases, a pump is used to force cell lysate through clarification filters. Flow rate of cell lysate through one or more filters may be modulated by adjusting one of channel size and/or fluid pressure.

According to some embodiments, cell lysates may be clarified by centrifugation. Centrifugation may be used to pellet insoluble particles in the lysate. During clarification, centrifugation strength [expressed in terms of gravitational units (g), which represents multiples of standard gravitational force] may be lower than in subsequent purification steps. In some cases, centrifugation may be carried out on cell lysates at from about 200 g to about 800 g, from about 500 g to about 1500 g, from about 1000 g to about 5000 g, from about 1200 g to about 10000 g or from about 8000 g to about 15000 g. In some embodiments, cell lysate centrifugation is carried out at 8000 g for 15 minutes. In some cases, density gradient centrifugation may be carried out in order to partition particulates in the cell lysate by sedimentation rate. Gradients used according to methods of the present disclosure may include, but are not limited to cesium chloride gradients and iodixanol step gradients.

### Purification: Chromatography

In some cases, AAV particles may be purified from clarified cell lysates by one or more methods of chromatography. Chromatography refers to any number of methods known in the art for separating out one or more elements from a mixture. Such methods may include, but are not limited to ion exchange chromatography (e.g. cation exchange chromatography and anion exchange chromatography), immunoaffinity chromatography and size-exclusion chromatography. In some embodiments, methods of viral chromatography may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

In some embodiments, ion exchange chromatography may be used to isolate viral particles. Ion exchange chromatography is used to bind viral particles based on charge-charge interactions between capsid proteins and charged sites present on a stationary phase, typically a column through which viral preparations (e.g. clarified lysates) are passed. After application of viral preparations, bound viral particles may then be eluted by applying an elution solution to disrupt the charge-charge interactions. Elution solutions may be optimized by adjusting salt concentration and/or pH to enhance recovery of bound viral particles. Depending on the charge of viral capsids being isolated, cation or anion exchange chromatography methods may be selected. Methods of ion exchange chromatography may include, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety.

In some embodiments, immunoaffinity chromatography may be used. Immunoaffinity chromatography is a form of chromatography that utilizes one or more immune compounds (e.g. antibodies or antibody-related structures) to retain viral particles. Immune compounds may bind specifically to one or more structures on viral particle surfaces, including, but not limited to one or more viral coat protein. In some cases, immune compounds may be specific for a particular viral variant. In some cases, immune compounds may bind to multiple viral variants. In some embodiments, immune compounds may include recombinant single-chain antibodies. Such recombinant single chain antibodies may include those described in Smith, R. H. et al., 2009. Mol. Ther. 17(11):1888-96, the contents of which are herein incorporated by reference in their entirety. Such immune compounds are capable of binding to several AAV capsid variants, including, but not limited to AAV1, AAV2, AAV6 and AAV8.

In some embodiments, size-exclusion chromatography (SEC) may be used. SEC may comprise the use of a gel to separate particles according to size. In viral particle purification, SEC filtration is sometimes referred to as “polishing.” In some cases, SEC may be carried out to generate a final product that is near-homogenous. Such final products may in some cases be used in pre-clinical studies and/or clinical studies (Kotin, R. M. 2011. Human Molecular Genetics. 20(1):R2-R6, the contents of which are herein incorporated by reference in their entirety.) In some cases, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

In one embodiment, the compositions comprising at least one AAV particle may be isolated or purified using the methods described in U.S. Pat. No. 6,146,874, the contents of which are herein incorporated by reference in its entirety.

In one embodiment, the compositions comprising at least one AAV particle may be isolated or purified using the methods described in U.S. Pat. No. 6,660,514, the contents of which are herein incorporated by reference in its entirety.

In one embodiment, the compositions comprising at least one AAV particle may be isolated or purified using the methods described in U.S. Pat. No. 8,283,151, the contents of which are herein incorporated by reference in its entirety.

In one embodiment, the compositions comprising at least one AAV particle may be isolated or purified using the methods described in U.S. Pat. No. 8,524,446, the contents of which are herein incorporated by reference in its entirety.

### II. Formulation and Delivery

### Pharmaceutical Compositions and Formulation

In addition to the pharmaceutical compositions (AAV particles comprising a modulatory polynucleotide sequence encoding the siRNA molecules), provided herein are pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients or subjects. For the purposes of the present disclosure, the phrase “active ingredient” generally refers either to the synthetic siRNA duplexes, the modulatory polynucleotide encoding the siRNA duplex, or the AAV particle comprising a modulatory polynucleotide encoding the siRNA duplex described herein.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered.

The AAV particles comprising the modulatory polynucleotide sequence encoding the siRNA molecules of the present invention can be formulated using one or more excipients to: (1) increase stability; (2) increase cell transfection or transduction; (3) permit the sustained or delayed release; or (4) alter the biodistribution (e.g., target the AAV particle to specific tissues or cell types such as brain and neurons).

Formulations of the present invention can include, without limitation, saline, lipidoids, liposomes, lipid nanoparticles, polymers, lipoplexes, core-shell nanoparticles, peptides, proteins, cells transfected with AAV particles (e.g., for transplantation into a subject), nanoparticle mimics and combinations thereof. Further, the AAV particles of the present invention may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

In some embodiments, the formulations may comprise at least one inactive ingredient. As used herein, the term “inactive ingredient” refers to one or more inactive agents included in formulations. In some embodiments, all, none or some of the inactive ingredients which may be used in the formulations of the present invention may be approved by the US Food and Drug Administration (FDA).

Formulations of vectors comprising the nucleic acid sequence for the siRNA molecules of the present invention may include cations or anions. In one embodiment, the formulations include metal cations such as, but not limited to, Zn2+, Ca2+, Cu2+, Mg+ and combinations thereof.

As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977); the content of each of which is incorporated herein by reference in their entirety.

The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

According to the present invention, the AAV particle comprising the modulatory polynucleotide sequence encoding for the siRNA molecules may be formulated for CNS delivery. Agents that cross the brain blood barrier may be used. For example, some cell penetrating peptides that can target siRNA molecules to the brain blood barrier endothelium may be used to formulate the siRNA duplexes targeting the gene of interest.

### Inactive Ingredients

In some embodiments, formulations may comprise at least one excipient which is an inactive ingredient. As used herein, the term “inactive ingredient” refers to one or more inactive agents included in formulations. In some embodiments, all, none or some of the inactive ingredients which may be used in the formulations of the present disclosure may be approved by the US Food and Drug Administration (FDA).

Formulations of AAV particles described herein may include cations or anions. In one embodiment, the formulations include metal cations such as, but not limited to, Zn2+, Ca2+, Cu2+, Mg+ and combinations thereof. As a non-limiting example, formulations may include polymers and compositions described herein complexed with a metal cation (See e.g., U.S. Pat. Nos. 6,265,389 and 6,555,525, each of which is herein incorporated by reference in its entirety).

### Delivery

In one embodiment, the AAV particles described herein may be administered or delivered using the methods for the delivery of AAV virions described in European Patent Application No. EP1857552, the contents of which are herein incorporated by reference in its entirety.

In one embodiment, the AAV particles described herein may be administered or delivered using the methods for delivering proteins using AAV particles described in European Patent Application No. EP2678433, the contents of which are herein incorporated by reference in its entirety.

In one embodiment, the AAV particle described herein may be administered or delivered using the methods for delivering DNA molecules using AAV particles described in U.S. Pat. No. 5,858,351, the contents of which are herein incorporated by reference in its entirety.

In one embodiment, the AAV particle described herein may be administered or delivered using the methods for delivering DNA to the bloodstream described in U.S. Pat. No. 6,211,163, the contents of which are herein incorporated by reference in its entirety.

In one embodiment, the AAV particle described herein may be administered or delivered using the methods for delivering AAV virions described in U.S. Pat. No. 6,325,998, the contents of which are herein incorporated by reference in its entirety.

In one embodiment, the AAV particle described herein may be administered or delivered using the methods for delivering a payload to the central nervous system described in U.S. Pat. No. 7,588,757, the contents of which are herein incorporated by reference in its entirety.

In one embodiment, the AAV particle described herein may be administered or delivered using the methods for delivering a payload described in U.S. Pat. No. 8,283,151, the contents of which are herein incorporated by reference in its entirety.

In one embodiment, the AAV particle described herein may be administered or delivered using the methods for delivering a payload using a glutamic acid decarboxylase (GAD) delivery vector described in International Patent Publication No. WO2001089583, the contents of which are herein incorporated by reference in its entirety.

In one embodiment, the AAV particle described herein may be administered or delivered using the methods for delivering a payload to neural cells described in International Patent Publication No. WO2012057363, the contents of which are herein incorporated by reference in its entirety.

### Delivery to Cells

The present disclosure provides a method of delivering to a cell or tissue any of the above-described AAV polynucleotides or AAV genomes, comprising contacting the cell or tissue with said AAV polynucleotide or AAV genomes or contacting the cell or tissue with a particle comprising said AAV polynucleotide or AAV genome, or contacting the cell or tissue with any of the described compositions, including pharmaceutical compositions. The method of delivering the AAV polynucleotide or AAV genome to a cell or tissue can be accomplished in vitro, ex vivo, or in vivo.

Introduction into Cells—Synthetic dsRNA

To ensure the chemical and biological stability of siRNA molecules (e.g., siRNA duplexes and dsRNA), it is important to deliver siRNA molecules inside the target cells. In some embodiments, the cells may include, but are not limited to, cells of mammalian origin, cells of human origins, embryonic stem cells, induced pluripotent stem cells, neural stem cells, and neural progenitor cells.

Nucleic acids, including siRNA, carry a net negative charge on the sugar-phosphate backbone under normal physiological conditions. In order to enter the cell, a siRNA molecule must come into contact with a lipid bilayer of the cell membrane, whose head groups are also negatively charged.

The siRNA duplexes can be complexed with a carrier that allows them to traverse cell membranes such as package particles to facilitate cellular uptake of the siRNA. The package particles may include, but are not limited to, liposomes, nanoparticles, cationic lipids, polyethylenimine derivatives, dendrimers, carbon nanotubes and the combination of carbon-made nanoparticles with dendrimers. Lipids may be cationic lipids and/or neutral lipids. In addition to well established lipophilic complexes between siRNA molecules and cationic carriers, siRNA molecules can be conjugated to a hydrophobic moiety, such as cholesterol (e.g., U.S. Patent Publication No. 20110110937; the content of which is herein incorporated by reference in its entirety). This delivery method holds a potential of improving in vitro cellular uptake and in vivo pharmacological properties of siRNA molecules. The siRNA molecules of the present invention may also be conjugated to certain cationic cell-penetrating peptides (CPPs), such as MPG, transportan or penetratin covalently or non-covalently (e.g., U.S. Patent Publication No. 20110086425; the content of which is herein incorporated by reference in its entirety).

Introduction into Cells—AAV Particles

The siRNA molecules (e.g., siRNA duplexes) of the present invention may be introduced into cells using any of a variety of approaches such as, but not limited to, AAV particles. These AAV particles are engineered and optimized to facilitate the entry of siRNA molecule into cells that are not readily amendable to transfection. Also, some synthetic AAV particles possess an ability to integrate the shRNA into the cell genome, thereby leading to stable siRNA expression and long-term knockdown of a target gene. In this manner, AAV particles are engineered as vehicles for specific delivery while lacking the deleterious replication and/or integration features found in wild-type virus.

In some embodiments, the siRNA molecules of the present invention are introduced into a cell by contacting the cell with an AAV particle comprising a modulatory polynucleotide sequence encoding a siRNA molecule, and a lipophilic carrier. In other embodiments, the siRNA molecule is introduced into a cell by transfecting or infecting the cell with an AAV particle comprising a nucleic acid sequence capable of producing the siRNA molecule when transcribed in the cell. In some embodiments, the siRNA molecule is introduced into a cell by injecting into the cell an AAV particle comprising a nucleic acid sequence capable of producing the siRNA molecule when transcribed in the cell.

In some embodiments, prior to transfection, an AAV particle comprising a nucleic acid sequence encoding the siRNA molecules of the present invention may be transfected into cells.

In other embodiments, the AAV particles comprising the nucleic acid sequence encoding the siRNA molecules of the present invention may be delivered into cells by electroporation (e.g. U.S. Patent Publication No. 20050014264; the content of which is herein incorporated by reference in its entirety).

Other methods for introducing AAV particles comprising the nucleic acid sequence encoding the siRNA molecules described herein may include photochemical internalization as described in U. S. Patent publication No. 20120264807; the content of which is herein incorporated by reference in its entirety.

In some embodiments, the formulations described herein may contain at least one AAV particle comprising the nucleic acid sequence encoding the siRNA molecules described herein. In one embodiment, the siRNA molecules may target the gene of interest at one target site. In another embodiment, the formulation comprises a plurality of AAV particles, each AAV particle comprising a nucleic acid sequence encoding a siRNA molecule targeting the gene of interest at a different target site. The gene of interest may be targeted at 2, 3, 4, 5 or more than 5 sites.

In one embodiment, the AAV particles from any relevant species, such as, but not limited to, human, dog, mouse, rat or monkey may be introduced into cells.

In one embodiment, the AAV particles may be introduced into cells which are relevant to the disease to be treated. As a non-limiting example, the disease is HD and the target cells are neurons and astrocytes. As another non-limiting example, the disease is HD and the target cells are medium spiny neurons, cortical neurons and astrocytes.

In one embodiment, the AAV particles may be introduced into cells which are relevant to the disease to be treated. As a non-limiting example, the disease is ALS and the target cells are neurons and astrocytes. As another non-limiting example, the disease is ALS and the target cells are medium spiny neurons, cortical neurons and astrocytes.

In one embodiment, the AAV particles may be introduced into cells which have a high level of endogenous expression of the target sequence.

In another embodiment, the AAV particles may be introduced into cells which have a low level of endogenous expression of the target sequence.

In one embodiment, the cells may be those which have a high efficiency of AAV transduction.

### Delivery to Subjects

The present disclosure additionally provides a method of delivering to a subject, including a mammalian subject, any of the above-described AAV polynucleotides or AAV genomes comprising administering to the subject said AAV polynucleotide or AAV genome, or administering to the subject a particle comprising said AAV polynucleotide or AAV genome, or administering to the subject any of the described compositions, including pharmaceutical compositions.

The pharmaceutical compositions of AAV particles described herein may be characterized by one or more of bioavailability, therapeutic window and/or volume of distribution.

### III. Administration and Dosing

### Administration

The AAV particles comprising a nucleic acid sequence encoding the siRNA molecules of the present invention may be administered by any route which results in a therapeutically effective outcome. These include, but are not limited to, within the parenchyma of an organ such as, but not limited to, a brain (e.g., intraparenchymal), corpus striatum (intrastriatal), enteral (into the intestine), gastroenteral, epidural, oral (by way of the mouth), transdermal, peridural, intracerebral (into the cerebrum), intracerebroventricular (into the cerebral ventricles), subpial (under the pia), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraganglionic (into the ganglion), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In specific embodiments, compositions of AAV particles comprising a nucleic acid sequence encoding the siRNA molecules of the present invention may be administered in a way which facilitates the vectors or siRNA molecule to enter the central nervous system and penetrate into medium spiny and/or cortical neurons and/or astrocytes.

In some embodiments, the AAV particles comprising a nucleic acid sequence encoding the siRNA molecules of the present invention may be administered by intramuscular injection.

In one embodiment, the AAV particles comprising a nucleic acid sequence encoding the siRNA molecules of the present invention may be administered via intraparenchymal injection.

In one embodiment, the AAV particles comprising a nucleic acid sequence encoding the siRNA molecules of the present invention may be administered via intraparenchymal injection and intrathecal injection.

In one embodiment, the AAV particles comprising a nucleic acid sequence encoding the siRNA molecules of the present invention may be administered via intrastriatal injection.

In one embodiment, the AAV particles comprising a nucleic acid sequence encoding the siRNA molecules of the present invention may be administered via intrastriatal injection and another route of administration described herein.

In some embodiments, AAV particles that express siRNA duplexes of the present invention may be administered to a subject by peripheral injections (e.g., intravenous) and/or intranasal delivery. It was disclosed in the art that the peripheral administration of AAV particles for siRNA duplexes can be transported to the central nervous system, for example, to the neurons (e.g., U.S. Patent Publication Nos. 20100240739; and 20100130594; the content of each of which is incorporated herein by reference in their entirety).

In other embodiments, compositions comprising at least one AAV particle comprising a nucleic acid sequence encoding the siRNA molecules of the present invention may be administered to a subject by intracranial delivery (See, e.g., U.S. Pat. No. 8,119,611; the content of which is incorporated herein by reference in its entirety).

The AAV particle comprising a nucleic acid sequence encoding the siRNA molecules of the present invention may be administered in any suitable form, either as a liquid solution or suspension, as a solid form suitable for liquid solution or suspension in a liquid solution. The siRNA duplexes may be formulated with any appropriate and pharmaceutically acceptable excipient.

The AAV particle comprising a nucleic acid sequence encoding the siRNA molecules of the present invention may be administered in a “therapeutically effective” amount, i.e., an amount that is sufficient to alleviate and/or prevent at least one symptom associated with the disease, or provide improvement in the condition of the subject.

In one embodiment, the AAV particle may be administered to the CNS in a therapeutically effective amount to improve function and/or survival for a subject with Huntington's Disease (HD). As a non-limiting example, the vector may be administered by direct infusion into the striatum.

In one embodiment, the AAV particle may be administered to a subject (e.g., to the CNS of a subject via intrathecal administration) in a therapeutically effective amount for the siRNA duplexes or dsRNA to target the medium spiny neurons, cortical neurons and/or astrocytes. As a non-limiting example, the siRNA duplexes or dsRNA may target HTT and reduce the expression of HTT protein or mRNA. As another non-limiting example, the siRNA duplexes or dsRNA target HTT and can suppress HTT and reduce HTT mediated toxicity. The reduction of HTT protein and/or mRNA as well as HTT mediated toxicity may be accomplished with almost no enhanced inflammation.

In one embodiment, the AAV particle may be administered to a subject (e.g., to the CNS of a subject) in a therapeutically effective amount to slow the functional decline of a subject (e.g., determined using a known evaluation method such as the unified Huntington's disease rating scale (UHDRS)). As a non-limiting example, the vector may be administered via intraparenchymal injection.

In one embodiment, the AAV particle may be administered to the cisterna magna in a therapeutically effective amount to transduce medium spiny neurons, cortical neurons and/or astrocytes. As a non-limiting example, the vector may be administered intrathecally.

In one embodiment, the AAV particle may be administered using intrathecal infusion in a therapeutically effective amount to transduce medium spiny neurons, cortical neurons and/or astrocytes. As a non-limiting example, the vector may be administered intrathecally.

In one embodiment, the AAV particle may be administered to the cisterna magna in a therapeutically effective amount to transduce medium spiny neurons, cortical neurons and/or astrocytes. As a non-limiting example, the vector may be administered by intraparenchymal injection.

In one embodiment, the AAV particle comprising a modulatory polynucleotide may be formulated. As a non-limiting example the baricity and/or osmolality of the formulation may be optimized to ensure optimal drug distribution in the central nervous system or a region or component of the central nervous system.

In one embodiment, the AAV particle comprising a modulatory polynucleotide may be delivered to a subject via a single route administration.

In one embodiment, the AAV particle comprising a modulatory polynucleotide may be delivered to a subject via a multi-site route of administration. A subject may be administered the AAV particle comprising a modulatory polynucleotide at 2, 3, 4, 5 or more than 5 sites.

In one embodiment, a subject may be administered the AAV particle comprising a modulatory polynucleotide described herein using a bolus injection.

In one embodiment, a subject may be administered the AAV particle comprising a modulatory polynucleotide described herein using sustained delivery over a period of minutes, hours or days. The infusion rate may be changed depending on the subject, distribution, formulation or another delivery parameter.

In one embodiment, the AAV particle described herein is administered via putamen and caudate infusion. As a non-limiting example, the dual infusion provides a broad striatal distribution as well as a frontal and temporal cortical distribution.

In one embodiment, the AAV particle is AAV-DJ8 which is administered via unilateral putamen infusion. As a non-limiting example, the distribution of the administered AAV-DJ8 is similar to the distribution of AAV1 delivered via unilateral putamen infusion.

In one embodiment, the AAV particle described herein is administered via intrathecal (IT) infusion at C1. The infusion may be for 1, 2, 3, 4, 6, 7, 8, 9, 10, 11, 12, 13, 14, 15 or more than 15 hours.

In one embodiment, the selection of subjects for administration of the AAV particle described herein and/or the effectiveness of the dose, route of administration and/or volume of administration may be evaluated using imaging of the perivascular spaces (PVS) which are also known as Virchow-Robin spaces. PVS surround the arterioles and venules as they perforate brain parenchyma and are filled with cerebrospinal fluid (CSF)/interstitial fluid. PVS are common in the midbrain, basal ganglia, and centrum semiovale. While not wishing to be bound by theory, PVS may play a role in the normal clearance of metabolites and have been associated with worse cognition and several disease states including Parkinson's disease. PVS are usually are normal in size but they can increase in size in a number of disease states. Potter et al. (Cerebrovasc Dis. 2015 January; 39(4): 224-231; the contents of which are herein incorporated by reference in its entirety) developed a grading method where they studied a full range of PVS and rated basal ganglia, centrum semiovale and midbrain PVS. They used the frequency and range of PVS used by Mac and Lullich et al. (J Neurol Neurosurg Psychiatry. 2004 November; 75(11):1519-23; the contents of which are herein incorporated by reference in its entirety) and Potter et al. gave 5 ratings to basal ganglia and centrum semiovale PVS: 0 (none), 1 (1-10), 2 (11-20), 3 (21-40) and 4 (>40) and 2 ratings to midbrain PVS: 0 (non visible) or 1 (visible). The user guide for the rating system by Potter et al. can be found at: www.sbirc.ed.ac.uk/documents/epvs-rating-scale-user-guide.pdf.

### Dosing

The pharmaceutical compositions of the present invention may be administered to a subject using any amount effective for reducing, preventing and/or treating a disease and/or disorder. The exact amount required will vary from subject to subject, depending on the species, age, and general condition of the subject, the severity of the disease, the particular composition, its mode of administration, its mode of activity, and the like.

The compositions of the present invention are typically formulated in unit dosage form for ease of administration and uniformity of dosage. It will be understood, however, that the total daily usage of the compositions of the present invention may be decided by the attending physician within the scope of sound medical judgment. The specific therapeutic effectiveness for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the siRNA duplexes employed; the duration of the treatment; drugs used in combination or coincidental with the specific compound employed; and like factors well known in the medical arts. \

In one embodiment, the age and sex of a subject may be used to determine the dose of the compositions of the present invention. As a non-limiting example, a subject who is older may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a younger subject. As another non-limiting example, a subject who is younger may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to an older subject. As yet another non-limiting example, a subject who is female may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a male subject. As yet another non-limiting example, a subject who is male may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a female subject

In some specific embodiments, the doses of AAV particles for delivering siRNA duplexes of the present invention may be adapted depending on the disease condition, the subject and the treatment strategy.

In one embodiment, delivery of the compositions in accordance with the present invention to cells comprises a rate of delivery defined by [VG/hour=mL/hour*VG/mL] wherein VG is viral genomes, VG/mL is composition concentration, and mL/hour is rate of prolonged delivery.

In one embodiment, delivery of compositions in accordance with the present invention to cells may comprise a total concentration per subject between about 1×10⁶ VG and about 1×10¹⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject.

In one embodiment, delivery of compositions in accordance with the present invention to cells may comprise a total concentration per subject between about 1×10⁶ VG/kg and about 1×10¹⁶ VG/kg. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/kg.

In one embodiment, about 10⁵ to 10⁶ viral genome (unit) may be administered per dose.

In one embodiment, delivery of the compositions in accordance with the present invention to cells may comprise a total concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL.

In certain embodiments, the desired siRNA duplex dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any modulatory polynucleotide therapeutic administered in one dose/at one time/single route/single point of contact, i.e., single administration event. As used herein, a “total daily dose” is an amount given or prescribed in a 24 hour period. It may be administered as a single unit dose. In one embodiment, the AAV particles comprising the modulatory polynucleotides of the present invention are administered to a subject in split doses. They may be formulated in buffer only or in a formulation described herein.

In one embodiment, the dose, concentration and/or volume of the composition described herein may be adjusted depending on the contribution of the caudate or putamen to cortical and subcortical distribution after administration. The administration may be intracerebroventricular, intraputamenal, intrathalamic, intraparenchymal, subpial, and/or intrathecal administration.

In one embodiment, the dose, concentration and/or volume of the composition described herein may be adjusted depending on the cortical and neuraxial distribution following administration by intracerebroventricular, intraputamenal, intrathalamic, intraparenchymal, subpial, and/or intrathecal delivery.

### IV. Methods and Uses of the Compositions of the Invention

### Huntington's Disease (HD)

Huntington's Disease (HD) is a monogenic fatal neurodegenerative disease characterized by progressive chorea, neuropsychiatric and cognitive dysfunction. Huntington's disease is known to be caused by an autosomal dominant triplet (CAG) repeat expansion in the huntingtin (HTT) gene, which encodes poly-glutamine at the N-terminus of the HTT protein. This repeat expansion results in a toxic gain of function of HTT and ultimately leads to striatal neurodegeneration which progresses to widespread brain atrophy. Medium spiny neurons of the striatum appear to be especially vulnerable in HD with up to 95% loss, whereas interneurons are largely spared.

Huntington's Disease has a profound impact on quality of life. Symptoms typically appear between the ages of 35-44 and life expectancy subsequent to onset is 10-25 years. In a small percentage of the HD population (˜6%), disease onset occurs prior to the age of 21 with appearance of an akinetic-rigid syndrome. These cases tend to progress faster than those of the later onset variety and have been classified as juvenile or Westphal variant HD. It is estimated that approximately 35,000-70,000 patients are currently suffering from HD in the US and Europe. Currently, only symptomatic relief and supportive therapies are available for treatment of HD, with a cure yet to be identified. Ultimately, individuals with HD succumb to pneumonia, heart failure or other complications such as physical injury from falls.

While not wishing to be bound by theory, the function of the wild type HTT protein may serve as a scaffold to coordinate complexes of other proteins. HTT is a very large protein (67 exons, 3144 amino acids, ˜350 kDa) that undergoes extensive post-translational modification and has numerous sites for interaction with other proteins, particularly at its N-terminus (coincidently the region that carries the repeats in HD). HTT localizes primarily to the cytoplasm but has been shown to shuttle into the nucleus where it may regulate gene transcription. It has also been suggested that HTT has a role in vesicular transport and regulating RNA trafficking.

As a non-limiting example, the HTT nucleic acid sequence is SEQ ID NO: 1163 (NCBI NM_002111.7).

The mechanisms by which CAG-expanded HTT disrupts normal HTT function and results in neurotoxicity were initially thought to be a disease of haploinsufficiency, this theory was disproven when terminal deletion of the HTT gene in man did not lead to development of HD, suggesting that fully expressed HTT protein is not critical to survival. However, conditional knockout of HTT in mouse led to neurodegeneration, indicating that some amount of HTT is necessary for cell survival. Huntingtin protein is expressed in all cells, though its concentration is highest in the brain where large aggregates of abnormal HTT are found in neuronal nuclei. In the brains of HD patients, HTT aggregates into abnormal nuclear inclusions. It is now believed that it is this process of misfolding and aggregating along with the associated protein intermediates (i.e. the soluble species and toxic N-terminal fragments) that result in neurotoxicity. In fact, HD belongs to a family of nine additional human genetic disorders all of which are characterized by CAG-expanded genes and resultant polyglutamine (poly-Q) protein products with subsequent formation of intraneuronal aggregates. Interestingly, in all of these diseases the length of the expansion correlates with both age of onset and rate of disease progression, with longer expansions linked to greater severity of disease.

Hypotheses on the molecular mechanisms underlying the neurotoxicity of CAG-expanded HTT and its resultant aggregates have been wide ranging, but include, caspase activation, dysregulation of transcriptional pathways, increased production of reactive oxygen species, mitochondrial dysfunction, disrupted axonal transport and/or inhibition of protein degradation systems within the cell. CAG-expanded HTT may not only have a toxic gain of function, but also exert a dominant negative effect by interfering with the normal function of other cellular proteins and processes. HTT has also been implicated in non-cell autonomous neurotoxicity, whereby a cell hosting HTT spreads the HTT to other neurons nearby.

In one embodiment, a subject has fully penetrant HD where the HTT gene has 41 or more CAG repeats (e.g., 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more than 90 CAG repeats).

In one embodiment, a subject has incomplete penetrance where the HTT gene has between 36 and 40 CAG repeats (e.g., 36, 37, 38, 39 and 40 CAG repeats).

Symptoms of HD may include features attributed to CNS degeneration such as, but are not limited to, chorea, dystonia, bradykinesia, incoordination, irritability and depression, problem solving difficulties, reduction in the ability of a person to function in their normal day to day life, diminished speech, and difficulty swallowing, as well as features not attributed to CNS degeneration such as, but not limited to, weight loss, muscle wasting, metabolic dysfunction and endocrine disturbances.

Model systems for studying Huntington's Disease which may be used with the modulatory polynucleotides and AAV particles described herein include, but are not limited to, cell models (e.g., primary neurons and induced pluripotent stem cells), invertebrate models (e.g., drosophila or Caenorhabditis elegans), mouse models (e.g., YAC128 mouse model; R6/2 mouse model; BAC, YAC and knock-in mouse model), rat models (e.g., BAC) and large mammal models (e.g., pigs, sheep or monkeys).

Studies in animal models of HD have suggested that phenotypic reversal is feasible, for example, subsequent to gene shut off in regulated-expression models. In a mouse model allowing shut off of expression of a 94-polyglutamine repeat HTT protein, not only was the clinical syndrome reversed but also the intracellular aggregates were resolved. Further, animal models in which silencing of HTT was tested, demonstrated promising results with the therapy being both well tolerated and showing potential therapeutic benefit.

Such siRNA mediated HTT expression inhibition may be used for treating HD. According to the present invention, methods for treating and/or ameliorating HD in a patient comprises administering to the patient an effective amount of AAV particles comprising a nucleic acid sequence encoding the siRNA molecules of the present invention into cells. The administration of the AAV particles comprising such a nucleic acid sequence will encode the siRNA molecules which cause the inhibition/silence of HTT gene expression.

In one embodiment, the AAV particles described herein may be used to reduce the amount of HTT in a subject in need thereof and thus provides a therapeutic benefit as described herein.

In certain aspects, the symptoms of HD include behavioral difficulties and symptoms such as, but not limited to, apathy or lack of initiative, dysphoria, irritability, agitation or anxiety, poor self-care, poor judgment, inflexibility, disinhibition, depression, suicidal ideation euphoria, aggression, delusions, compulsions, hypersexuality, hallucinations, speech deterioration, slurred speech, difficulty swallowing, weight loss, cognitive dysfunction which impairs executive functions (e.g., organizing, planning, checking or adapting alternatives, and delays in the acquisition of new motor skills), unsteady gait and involuntary movements (chorea). In other aspects, the composition of the present invention is applied to one or both of the brain and the spinal cord. In one embodiment, the survival of the subject is prolonged by treating any of the symptoms of HD described herein.

Disclosed in the present invention are methods for treating Huntington's Disease (HD) associated with HTT protein in a subject in need of treatment. The method optionally comprises administering to the subject a therapeutically effective amount of a composition comprising at least AAV particles comprising a nucleic acid sequence encoding the siRNA molecules of the present invention. As a non-limiting example, the siRNA molecules can silence HTT gene expression, inhibit HTT protein production, and reduce one or more symptoms of HD in the subject such that HD is therapeutically treated.

### Methods of Treatment of Huntington's Disease

The present invention provides AAV particles comprising modulatory polynucleotides encoding siRNA molecules targeting the HTT gene, and methods for their design and manufacture. While not wishing to be bound by a single theory of operability, the invention provides modulatory polynucleotides, including siRNAs, that interfere with HTT expression, including HTT mutant and/or wild-type HTT gene expression. Particularly, the present invention employs viral genomes such as adeno-associated viral (AAV) viral genomes comprising modulatory polynucleotide sequences encoding the siRNA molecules of the present invention. The AAV particles comprising the modulatory polynucleotides encoding the siRNA molecules of the present invention may increase the delivery of active agents into neurons of interest such as medium spiny neurons of the striatum and cortical neurons. The siRNA duplexes or encoded dsRNA targeting the HTT gene may be able to inhibit HTT gene expression (e.g., mRNA level) significantly inside cells; therefore, reducing HTT expression induced stress inside the cells such as aggregation of protein and formation of inclusions, increased free radicals, mitochondrial dysfunction and RNA metabolism.

Provided in the present invention are methods for introducing the AAV particles comprising a modulatory polynucleotide sequence encoding the siRNA molecules of the present invention into cells, the method comprising introducing into said cells any of the AAV particles in an amount sufficient for degradation of target HTT mRNA to occur, thereby activating target-specific RNAi in the cells. In some aspects, the cells may be stem cells, neurons such as medium spiny or cortical neurons, muscle cells and glial cells such as astrocytes.

In some embodiments, the present invention provides methods for treating or ameliorating Huntington's Disease (HD) by administering to a subject in need thereof a therapeutically effective amount of a plasmid or AAV particle described herein.

In some embodiments, the AAV particles comprising modulatory polynucleotides encoding the siRNA molecules of the present invention may be used to treat and/or ameliorate for HD.

In one embodiment, the AAV particles comprising modulatory polynucleotides encoding the siRNA molecules of the present invention may be used to reduce the cognitive and/or motor decline of a subject with HD, where the amount of decline is determined by a standard evaluation system such as, but not limited to, Unified Huntington's Disease Ratings Scale (UHDRS) and subscores, and cognitive testing.

In one embodiment, the AAV particles comprising modulatory polynucleotides encoding the siRNA molecules of the present invention may be used to reduce the decline of functional capacity and activities of daily living as measured by a standard evaluation system such as, but not limited to, the total functional capacity (TFC) scale.

In some embodiments, the present invention provides methods for treating, or ameliorating Huntington's Disease associated with HTT gene and/or HTT protein in a subject in need of treatment, the method comprising administering to the subject a pharmaceutically effective amount of AAV particles comprising modulatory polynucleotides encoding at least one siRNA duplex targeting the HTT gene, inhibiting HTT gene expression and protein production, and ameliorating symptoms of HD in the subject.

In one embodiment, the AAV particles of the present invention may be used as a method of treating Huntington's disease in a subject in need of treatment. Any method known in the art for defining a subject in need of treatment may be used to identify said subject(s). A subject may have a clinical diagnosis of Huntington's disease, or may be pre-symptomatic. Any known method for diagnosing HD may be utilized, including, but not limited to, cognitive assessments and/or neurological or neuropsychiatric examinations, motor tests, sensory tests, psychiatric evaluations, brain imaging, family history and/or genetic testing.

In one embodiment, HD subject selection is determined with the use of the Prognostic Index for Huntington's Disease, or a derivative thereof (Long J D et al., Movement Disorders, 2017, 32(2), 256-263, the contents of which are herein incorporated by reference in their entirety). This prognostic index uses four components to predict probability of motor diagnosis, (1) total motor score (TMS) from the Unified Huntington's Disease Rating Scale (UHDRS), (2) Symbol Digit Modality Test (SDMT), (3) base-line age, and (4) cytosine-adenine-guanine (CAG) expansion.

In one embodiment, the prognostic index for Huntington's Disease is calculated with the following formula: PI_{HD}=51×TMS+(−34)×SDMT+7×Age×(CAG-34), wherein larger values for PI_{HD} indicate greater risk of diagnosis or onset of symptoms.

In another embodiment, the prognostic index for Huntington's Disease is calculated with the following normalized formula that gives standard deviation units to be interpreted in the context of 50% 10-year survival: PIN_{HD}=(PI_{ID}−883)/1044, wherein PIN_{HD}<0 indicates greater than 50% 10-year survival, and PIN_{HD}>0 suggests less than 50% 10-year survival.

In one embodiment, the prognostic index may be used to identify subjects whom will develop symptoms of HD within several years, but that do not yet have clinically diagnosable symptoms. Further, these asymptomatic patients may be selected for and receive treatment using the AAV particles and compositions of the present invention during the asymptomatic period.

In one embodiment, the AAV particles may be administered to a subject who has undergone biomarker assessment. Potential biomarkers in blood for premanifest and early progression of HD include, but are not limited to, 8-OhdG oxidative stress marker, metabolic markers (e.g., creatine kinase, branched-chain amino acids), cholesterol metabolites (e.g., 24-OH cholesterol), immune and inflammatory proteins (e.g., clusterin, complement components, interleukins 6 and 8), gene expression changes (e.g., transcriptomic markers), endocrine markers (e.g., cortisol, ghrelin and leptin), BDNF, adenosine 2A receptors. Potential biomarkers for brain imaging for premanifest and early progression of HD include, but are not limited to, striatal volume, subcortical white-matter volume, cortical thickness, whole brain and ventricular volumes, functional imaging (e.g., functional MRI), PET (e.g., with fluorodeoxyglucose), and magnetic resonance spectroscopy (e.g., lactate). Potential biomarkers for quantitative clinical tools for premanifest and early progression of HD include, but are not limited to, quantitative motor assessments, motor physiological assessments (e.g., transcranial magnetic stimulation), and quantitative eye movement measurements. Non-limiting examples of quantitative clinical biomarker assessments include tongue force variability, metronome-guided tapping, grip force, oculomotor assessments and cognitive tests. Non-limiting examples of multicenter observational studies include PREDICT-HD and TRACK-HD. A subject may have symptoms of HD, diagnosed with HD or may be asymptomatic for HD.

In one embodiment, the AAV particles may be administered to a subject who has undergone biomarker assessment using neuroimaging. A subject may have symptoms of HD, diagnosed with HD or may be asymptomatic for HD.

In one embodiment, the AAV particles may be administered to a subject who is asymptomatic for HD. A subject may be asymptomatic but may have undergone predictive genetic testing or biomarker assessment to determine if they are at risk for HD and/or a subject may have a family member (e.g., mother, father, brother, sister, aunt, uncle, grandparent) who has been diagnosed with HD.

In one embodiment, the AAV particles may be administered to a subject who is in the early stages of HD. In the early stage a subject has subtle changes in coordination, some involuntary movements (chorea), changes in mood such as irritability and depression, problem solving difficulties, reduction in the ability of a person to function in their normal day to day life.

In one embodiment, the AAV particles may be administered to a subject who is in the middle stages of HD. In the middle stage a subject has an increase in the movement disorder, diminished speech, difficulty swallowing, and ordinary activities will become harder to do. At this stage a subject may have occupational and physical therapists to help maintain control of voluntary movements and a subject may have a speech language pathologist.

In one embodiment, the AAV particles may be administered to a subject who is in the late stages of HD. In the late stage, a subject with HD is almost completely or completely dependent on others for care as the subject can no longer walk and is unable to speak. A subject can generally still comprehend language and is aware of family and friends but choking is a major concern.

In one embodiment, the AAV particles may be used to treat a subject who has the juvenile form of HD which is the onset of HD before the age of 20 years and as early as 2 years.

In one embodiment, the AAV particles may be used to treat a subject with HD who has fully penetrant HD where the HTT gene has 41 or more CAG repeats (e.g., 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more than 90 CAG repeats).

In one embodiment, the AAV particles may be used to treat a subject with HD who has incomplete penetrance where the HTT gene has between 36 and 40 CAG repeats (e.g., 36, 37, 38, 39 and 40 CAG repeats).

In some embodiments, the composition comprising the AAV particles comprising modulatory polynucleotides encoding the siRNA molecules of the present invention is administered to the central nervous system of the subject. In other embodiments, the composition comprising the AAV particles comprising modulatory polynucleotides encoding the siRNA molecules of the present invention is administered to a tissue of a subject (e.g., brain of the subject).

In one embodiment, the AAV particles comprising modulatory polynucleotides encoding the siRNA molecules of the present invention may be delivered into specific types of targeted cells, including, but not limited to, neurons including medium spiny or cortical neurons; glial cells including oligodendrocytes, astrocytes and microglia; and/or other cells surrounding neurons such as T cells.

In one embodiment, the AAV particles comprising modulatory polynucleotides encoding the siRNA molecules of the present invention may be delivered to neurons in the striatum and/or neurons of the cortex.

In some embodiments, the composition of the present invention for treating HD is administered to the subject in need intravenously, intramuscularly, subcutaneously, intraperitoneally, intraparenchymally, subpially, intrathecally and/or intraventricularly, allowing the siRNA molecules or vectors comprising the siRNA molecules to pass through one or both the blood-brain barrier and the blood spinal cord barrier, or directly access the brain and/or spinal cord. In some aspects, the method includes administering (e.g., intraparenchymal administration, subpial administration, intraventricular administration and/or intrathecal administration) directly to the central nervous system (CNS) of a subject (using, e.g., an infusion pump and/or a delivery scaffold) a therapeutically effective amount of a composition comprising AAV particles encoding the nucleic acid sequence for the siRNA molecules of the present invention. The vectors may be used to silence or suppress HTT gene expression, and/or reducing one or more symptoms of HD in the subject such that HD is therapeutically treated.

In some embodiments, the siRNA molecules or the AAV particles comprising such siRNA molecules may be introduced directly into the central nervous system of the subject, for example, by infusion to the white matter a subject. While not wishing to be bound by theory, distribution via direct white matter infusion may be independent of axonal transport mechanisms which may be impaired in subjects with Huntington's Disease which means white matter infusion may allow for more transport of the AAV particles.

In one embodiment, the composition comprising the AAV particles comprising modulatory polynucleotides encoding the siRNA molecules of the present invention is administered to the central nervous system of the subject via intraparenchymal injection.

In one embodiment, the AAV particle composition comprising modulatory polynucleotides encoding the siRNA molecules of the present invention is administered to the central nervous system of the subject via intraparenchymal injection and intrathecal injection.

In one embodiment, the AAV particle composition comprising modulatory polynucleotides encoding the siRNA molecules of the present invention is administered to the central nervous system of the subject via intraparenchymal injection and intracerebroventricular injection.

In some embodiments, the composition of the present invention for treating HD is administered to the subject in need by intraparenchymal administration.

In some embodiments, the AAV particle composition comprising modulatory polynucleotides encoding the siRNA molecules of the present invention may be introduced directly into the central nervous system of the subject, for example, by infusion into the putamen.

In some embodiments, the AAV particle composition comprising modulatory polynucleotides encoding the siRNA molecules of the present invention may be introduced directly into the central nervous system of the subject, for example, by infusion into the thalamus of a subject. While not wishing to be bound by theory, the thalamus is an area of the brain which is relatively spared in subjects with Huntington's Disease which means it may allow for more widespread cortical transduction via axonal transport of the AAV particles.

In some embodiments, the AAV particle composition comprising modulatory polynucleotides encoding the siRNA molecules of the present invention may be introduced indirectly into the central nervous system of the subject, for example, by intravenous administration.

### Modulate HTT Expression

In one embodiment, administration of the AAV particles to a subject will reduce the expression of HTT in a subject and the reduction of expression of the HTT will reduce the effects of HD in a subject.

In one embodiment, the encoded dsRNA once expressed and contacts a cell expressing HTT protein, inhibits the expression of HTT protein by at least 10%, at least 20%, at least 25%, at least 30%, at least 35% or at least 40% or more, such as when assayed by a method as described herein.

In one embodiment, administration of the AAV particles comprising a modulatory polynucleotide sequence encoding a siRNA of the invention, to a subject may lower HTT (e.g., mutant HTT, wild-type HTT and/or mutant and wild-type HTT) in a subject. In one embodiment, administration of the AAV particles to a subject may lower wild-type HTT in a subject. In yet another embodiment, administration of the AAV particles to a subject may lower both mutant HTT and wild-type HTT in a subject. The mutant and/or wild-type HTT may be lowered by about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. The mutant HTT may be lowered by about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. The wild-type HTT may be lowered by about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. The mutant and wild-type HTT may be lowered by about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the AAV particles may lower the expression of HTT by at least 50% in the medium spiny neurons. As a non-limiting example, the AAV particles may lower the expression of HTT by at least 40% in the medium spiny neurons. As a non-limiting example, the AAV particles may lower the expression of HTT by at least 40% in the medium spiny neurons of the putamen. As a non-limiting example, the AAV particles may lower the expression of HTT by at least 30% in the medium spiny neurons of the putamen. As yet another non-limiting example, the AAV particles may lower the expression of HTT in the putamen and cortex by at least 40%. As yet another non-limiting example, the AAV particles may lower the expression of HTT in the putamen and cortex by at least 30%. As yet another non-limiting example, the AAV particles may lower the expression of HTT in the putamen by at least 30%. As yet another non-limiting example, the AAV particles may lower the expression of HTT in the putamen by at least 30% and cortex by at least 15%.

In one embodiment, the AAV particles may be used to reduce the expression of HTT protein by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein expression may be reduced by 50-90%. As a non-limiting example, the expression of HTT protein expression may be reduced by 30-70%.

In one embodiment, the siRNA duplexes or encoded dsRNA may be used to reduce the expression of HTT mRNA by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT mRNA may be reduced 50-90%.

In one embodiment, the AAV particles may be used to decrease HTT protein in a subject. The decrease may independently be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. As a non-limiting example, a subject may have a 50% decrease of HTT protein. As a non-limiting example, a subject may have a decrease of 70% of HTT protein and a decrease of 10% of wild type HTT protein. As a non-limiting example, the decrease of HTT in the medium spiny neurons of the putamen may be about 40%. As a non-limiting example, the decrease of HTT in the putamen and cortex may be about 40%. As a non-limiting example, the decrease of HTT in the medium spiny neurons of the putamen may be between 40%-70%. As a non-limiting example, the decrease of HTT in the putamen and cortex may be between 40%-70%.

In one embodiment, the AAV particles may be used to decrease wild type HTT protein in a subject. The decrease may independently be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. As a non-limiting example, a subject may have a 50% decrease of wild type HTT protein. As a non-limiting example, the decrease of wild type HTT in the medium spiny neurons of the putamen may be about 40%. As a non-limiting example, the decrease of wild type HTT in the putamen and cortex may be about 40%. As a non-limiting example, the decrease of wild type HTT in the medium spiny neurons of the putamen may be between 40%-70%. As a non-limiting example, the decrease of wild type HTT in the putamen and cortex may be between 40%-70%.

In one embodiment, the AAV particles may be used to decrease mutant HTT protein in a subject. The decrease may independently be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. As a non-limiting example, a subject may have a 50% decrease of mutant HTT protein. As a non-limiting example, the decrease of mutant HTT in the medium spiny neurons of the putamen may be about 40%. As a non-limiting example, the decrease of mutant HTT in the putamen and cortex may be about 40%. As a non-limiting example, the decrease of mutant HTT in the medium spiny neurons of the putamen may be between 40%-70%. As a non-limiting example, the decrease of mutant HTT in the putamen and cortex may be between 40%-70%.\

In some embodiments, the present invention provides methods for inhibiting/silencing HTT gene expression in a cell. Accordingly, the siRNA duplexes or encoded dsRNA can be used to substantially inhibit HTT gene expression in a cell, in particular in a neuron. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the present invention provides methods for inhibiting/silencing HTT gene expression in a cell, in particular in a medium spiny neuron. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the present invention provides methods for inhibiting/silencing HTT gene expression in a cell, in particular in an astrocyte. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In one embodiment, the siRNA duplexes or encoded dsRNA may be used to reduce the expression of HTT protein and/or mRNA in at least one region of the CNS such as, but not limited to the midbrain. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in at least one region of the CNS. As a non-limiting example, the expression of HTT protein and mRNA in the striatum and/or cortex is reduced by 50-90%. As a non-limiting example, the expression of HTT protein and mRNA in the striatum is reduced by 40-50%. As a non-limiting example, the expression of HTT protein and mRNA in the cortex is reduced by 40-50%. As a non-limiting example, the expression of HTT protein and mRNA in the cortex is reduced by 30-70%. As a non-limiting example, the expression of HTT protein and mRNA in the striatum and/or cortex is reduced by 40-70%. As a non-limiting example, the expression of HTT protein and mRNA in the striatum and/or cortex is reduced by 40-50%. As a non-limiting example, the expression of HTT protein and mRNA in the striatum and/or cortex is reduced by 50-70%. As a non-limiting example, the expression of HTT protein and mRNA in the striatum and/or cortex is reduced by 50-60%. As a non-limiting example, the expression of HTT protein and mRNA in the striatum and/or cortex is reduced by 50%. As a non-limiting example, the expression of HTT protein and mRNA in the striatum and/or cortex is reduced by 51%. As a non-limiting example, the expression of HTT protein and mRNA in the striatum and/or cortex is reduced by 52%. As a non-limiting example, the expression of HTT protein and mRNA in the striatum and/or cortex is reduced by 53%. As a non-limiting example, the expression of HTT protein and mRNA in the striatum and/or cortex is reduced by 54%. As a non-limiting example, the expression of HTT protein and mRNA in the striatum and/or cortex is reduced by 55%. As a non-limiting example, the expression of HTT protein and mRNA in the striatum and/or cortex is reduced by 56%. As a non-limiting example, the expression of HTT protein and mRNA in the striatum and/or cortex is reduced by 57%. As a non-limiting example, the expression of HTT protein and mRNA in the striatum and/or cortex is reduced by 58%. As a non-limiting example, the expression of HTT protein and mRNA in the striatum and/or cortex is reduced by 59%. As a non-limiting example, the expression of HTT protein and mRNA in the striatum and/or cortex is reduced by 60%.

In one embodiment, the siRNA duplexes or encoded dsRNA may be used to reduce the expression of HTT protein and/or mRNA in at least one region of the CNS such as, but not limited to the forebrain. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in at least one region of the CNS. As a non-limiting example, the expression of HTT protein and mRNA in the putamen is reduced by 50-90%. As a non-limiting example, the expression of HTT protein and mRNA in the striatum is reduced by 40-50%. As a non-limiting example, the expression of HTT protein and mRNA in the cortex is reduced by 40-50%. As a non-limiting example, the expression of HTT protein and mRNA in the cortex is reduced by 30-70%. As a non-limiting example, the expression of HTT protein and mRNA in the striatum and/or cortex is reduced by 40-70%. As a non-limiting example, the expression of HTT protein and mRNA in the striatum and/or cortex is reduced by 40-50%. As a non-limiting example, the expression of HTT protein and mRNA in the striatum and/or cortex is reduced by 50-70%. As a non-limiting example, the expression of HTT protein and mRNA in the striatum and/or cortex is reduced by 50-60%. As a non-limiting example, the expression of HTT protein and mRNA in the striatum and/or cortex is reduced by 50%. As a non-limiting example, the expression of HTT protein and mRNA in the striatum and/or cortex is reduced by 51%. As a non-limiting example, the expression of HTT protein and mRNA in the striatum and/or cortex is reduced by 52%. As a non-limiting example, the expression of HTT protein and mRNA in the striatum and/or cortex is reduced by 53%. As a non-limiting example, the expression of HTT protein and mRNA in the striatum and/or cortex is reduced by 54%. As a non-limiting example, the expression of HTT protein and mRNA in the striatum and/or cortex is reduced by 55%. As a non-limiting example, the expression of HTT protein and mRNA in the striatum and/or cortex is reduced by 56%. As a non-limiting example, the expression of HTT protein and mRNA in the striatum and/or cortex is reduced by 57%. As a non-limiting example, the expression of HTT protein and mRNA in the striatum and/or cortex is reduced by 58%. As a non-limiting example, the expression of HTT protein and mRNA in the striatum and/or cortex is reduced by 59%. As a non-limiting example, the expression of HTT protein and mRNA in the striatum and/or cortex is reduced by 60%.

In one embodiment, the siRNA duplexes or encoded dsRNA may be used to reduce the expression of HTT protein and/or mRNA in the striatum. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and mRNA in the striatum is reduced by 40-50%. As a non-limiting example, the expression of HTT protein and mRNA in the striatum is reduced by 30-70%. As a non-limiting example, the expression of HTT protein and mRNA in the striatum is reduced by at least 30%. As a non-limiting example, the expression of HTT protein and mRNA in the striatum is reduced by 40-70%. As a non-limiting example, the expression of HTT protein and mRNA in the striatum is reduced by 40-50%. As a non-limiting example, the expression of HTT protein and mRNA in the striatum is reduced by 50-70%. As a non-limiting example, the expression of HTT protein and mRNA in the striatum is reduced by 50-60%. As a non-limiting example, the expression of HTT protein and mRNA in the striatum is reduced by 50%. As a non-limiting example, the expression of HTT protein and mRNA in the striatum is reduced by 51%. As a non-limiting example, the expression of HTT protein and mRNA in the striatum is reduced by 52%. As a non-limiting example, the expression of HTT protein and mRNA in the striatum is reduced by 53%. As a non-limiting example, the expression of HTT protein and mRNA in the striatum is reduced by 54%. As a non-limiting example, the expression of HTT protein and mRNA in the striatum is reduced by 55%. As a non-limiting example, the expression of HTT protein and mRNA in the striatum is reduced by 56%. As a non-limiting example, the expression of HTT protein and mRNA in the striatum is reduced by 57%. As a non-limiting example, the expression of HTT protein and mRNA in the striatum is reduced by 58%. As a non-limiting example, the expression of HTT protein and mRNA in the striatum is reduced by 59%. As a non-limiting example, the expression of HTT protein and mRNA in the striatum is reduced by 60%.

In some embodiments, the AAV particles comprising modulatory polynucleotides encoding the siRNA molecules of the present invention may be used to suppress HTT protein in neurons and/or astrocytes of the striatum and/or the cortex. As a non-limiting example, the suppression of HTT protein is in medium spiny neurons of the striatum and/or neurons of the cortex.

In some embodiments, the AAV particles comprising modulatory polynucleotides encoding the siRNA molecules of the present invention may be used to suppress HTT protein in neurons and/or astrocytes of the striatum and/or the cortex and reduce associated neuronal toxicity. The suppression of HTT protein in the neurons and/or astrocytes of the striatum and/or the cortex may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction of associated neuronal toxicity may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In one embodiment, the siRNA duplexes or encoded dsRNA may be used to reduce the expression of HTT protein and/or mRNA in the cortex. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and mRNA in the cortex is reduced by 40-50%. As a non-limiting example, the expression of HTT protein and mRNA in the cortex is reduced by 30-70%. As a non-limiting example, the expression of HTT protein and mRNA in the cortex is reduced by at least 30%. As a non-limiting example, the expression of HTT protein and mRNA in the cortex is reduced by 40-70%. As a non-limiting example, the expression of HTT protein and mRNA in the cortex is reduced by 40-50%. As a non-limiting example, the expression of HTT protein and mRNA in the cortex is reduced by 50-70%. As a non-limiting example, the expression of HTT protein and mRNA in the cortex is reduced by 50-60%. As a non-limiting example, the expression of HTT protein and mRNA in the cortex is reduced by 50%. As a non-limiting example, the expression of HTT protein and mRNA in the cortex is reduced by 51%. As a non-limiting example, the expression of HTT protein and mRNA in the cortex is reduced by 52%. As a non-limiting example, the expression of HTT protein and mRNA in the cortex is reduced by 53%. As a non-limiting example, the expression of HTT protein and mRNA in the cortex is reduced by 54%. As a non-limiting example, the expression of HTT protein and mRNA in the cortex is reduced by 55%. As a non-limiting example, the expression of HTT protein and mRNA in the cortex is reduced by 56%. As a non-limiting example, the expression of HTT protein and mRNA in the cortex is reduced by 57%. As a non-limiting example, the expression of HTT protein and mRNA in the cortex is reduced by 58%. As a non-limiting example, the expression of HTT protein and mRNA in the cortex is reduced by 59%. As a non-limiting example, the expression of HTT protein and mRNA in the cortex is reduced by 60%.

In one embodiment, the siRNA duplexes or encoded dsRNA may be used to reduce the expression of HTT protein and/or mRNA in the motor cortex. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and mRNA in the motor cortex is reduced by 40-50%. As a non-limiting example, the expression of HTT protein and mRNA in the motor cortex is reduced by 30-70%. As a non-limiting example, the expression of HTT protein and mRNA in the motor cortex is reduced by at least 30%. As a non-limiting example, the expression of HTT protein and mRNA in the motor cortex is reduced by 40-70%. As a non-limiting example, the expression of HTT protein and mRNA in the motor cortex is reduced by 40-50%. As a non-limiting example, the expression of HTT protein and mRNA in the motor cortex is reduced by 50-70%. As a non-limiting example, the expression of HTT protein and mRNA in the motor cortex is reduced by 50-60%. As a non-limiting example, the expression of HTT protein and mRNA in the motor cortex is reduced by 50%. As a non-limiting example, the expression of HTT protein and mRNA in the motor cortex is reduced by 51%. As a non-limiting example, the expression of HTT protein and mRNA in the motor cortex is reduced by 52%. As a non-limiting example, the expression of HTT protein and mRNA in the motor cortex is reduced by 53%. As a non-limiting example, the expression of HTT protein and mRNA in the motor cortex is reduced by 54%. As a non-limiting example, the expression of HTT protein and mRNA in the motor cortex is reduced by 55%. As a non-limiting example, the expression of HTT protein and mRNA in the motor cortex is reduced by 56%. As a non-limiting example, the expression of HTT protein and mRNA in the motor cortex is reduced by 57%. As a non-limiting example, the expression of HTT protein and mRNA in the motor cortex is reduced by 58%. As a non-limiting example, the expression of HTT protein and mRNA in the motor cortex is reduced by 59%. As a non-limiting example, the expression of HTT protein and mRNA in the motor cortex is reduced by 60%.

In one embodiment, the siRNA duplexes or encoded dsRNA may be used to reduce the expression of HTT protein and/or mRNA in the somatosensory cortex. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and mRNA in the somatosensory cortex is reduced by 40-50%. As a non-limiting example, the expression of HTT protein and mRNA in the somatosensory cortex is reduced by 30-70%. As a non-limiting example, the expression of HTT protein and mRNA in the somatosensory cortex is reduced by at least 30%. As a non-limiting example, the expression of HTT protein and mRNA in the somatosensory cortex is reduced by 40-70%. As a non-limiting example, the expression of HTT protein and mRNA in the somatosensory cortex is reduced by 40-50%. As a non-limiting example, the expression of HTT protein and mRNA in the somatosensory cortex is reduced by 50-70%. As a non-limiting example, the expression of HTT protein and mRNA in the somatosensory cortex is reduced by 50-60%. As a non-limiting example, the expression of HTT protein and mRNA in the somatosensory cortex is reduced by 50%. As a non-limiting example, the expression of HTT protein and mRNA in the somatosensory cortex is reduced by 51%. As a non-limiting example, the expression of HTT protein and mRNA in the somatosensory cortex is reduced by 52%. As a non-limiting example, the expression of HTT protein and mRNA in the somatosensory cortex is reduced by 53%. As a non-limiting example, the expression of HTT protein and mRNA in the somatosensory cortex is reduced by 54%. As a non-limiting example, the expression of HTT protein and mRNA in the somatosensory cortex is reduced by 55%. As a non-limiting example, the expression of HTT protein and mRNA in the somatosensory cortex is reduced by 56%. As a non-limiting example, the expression of HTT protein and mRNA in the somatosensory cortex is reduced by 57%. As a non-limiting example, the expression of HTT protein and mRNA in the somatosensory cortex is reduced by 58%. As a non-limiting example, the expression of HTT protein and mRNA in the somatosensory cortex is reduced by 59%. As a non-limiting example, the expression of HTT protein and mRNA in the somatosensory cortex is reduced by 60%.

In one embodiment, the siRNA duplexes or encoded dsRNA may be used to reduce the expression of HTT protein and/or mRNA in the temporal cortex. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and mRNA in the temporal cortex is reduced by 40-50%. As a non-limiting example, the expression of HTT protein and mRNA in the temporal cortex is reduced by 30-70%. As a non-limiting example, the expression of HTT protein and mRNA in the temporal cortex is reduced by at least 30%. As a non-limiting example, the expression of HTT protein and mRNA in the temporal cortex is reduced by 40-70%. As a non-limiting example, the expression of HTT protein and mRNA in the temporal cortex is reduced by 40-50%. As a non-limiting example, the expression of HTT protein and mRNA in the temporal cortex is reduced by 50-70%. As a non-limiting example, the expression of HTT protein and mRNA in the temporal cortex is reduced by 50-60%. As a non-limiting example, the expression of HTT protein and mRNA in the temporal cortex is reduced by 50%. As a non-limiting example, the expression of HTT protein and mRNA in the temporal cortex is reduced by 51%. As a non-limiting example, the expression of HTT protein and mRNA in the temporal cortex is reduced by 52%. As a non-limiting example, the expression of HTT protein and mRNA in the temporal cortex is reduced by 53%. As a non-limiting example, the expression of HTT protein and mRNA in the temporal cortex is reduced by 54%. As a non-limiting example, the expression of HTT protein and mRNA in the temporal cortex is reduced by 55%. As a non-limiting example, the expression of HTT protein and mRNA in the temporal cortex is reduced by 56%. As a non-limiting example, the expression of HTT protein and mRNA in the temporal cortex is reduced by 57%. As a non-limiting example, the expression of HTT protein and mRNA in the temporal cortex is reduced by 58%. As a non-limiting example, the expression of HTT protein and mRNA in the temporal cortex is reduced by 59%. As a non-limiting example, the expression of HTT protein and mRNA in the temporal cortex is reduced by 60%.

In one embodiment, the siRNA duplexes or encoded dsRNA may be used to reduce the expression of HTT protein and/or mRNA in the putamen. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in at least one region of the CNS. As a non-limiting example, the expression of HTT protein and mRNA in the putamen is reduced by 40-70%. As a non-limiting example, the expression of HTT protein and mRNA in the putamen is reduced by 40-50%. As a non-limiting example, the expression of HTT protein and mRNA in the putamen is reduced by 50-70%. As a non-limiting example, the expression of HTT protein and mRNA in the putamen is reduced by 50-60%. As a non-limiting example, the expression of HTT protein and mRNA in the putamen is reduced by 50%. As a non-limiting example, the expression of HTT protein and mRNA in the putamen is reduced by 51%. As a non-limiting example, the expression of HTT protein and mRNA in the putamen is reduced by 52%. As a non-limiting example, the expression of HTT protein and mRNA in the putamen is reduced by 53%. As a non-limiting example, the expression of HTT protein and mRNA in the putamen is reduced by 54%. As a non-limiting example, the expression of HTT protein and mRNA in the putamen is reduced by 55%. As a non-limiting example, the expression of HTT protein and mRNA in the putamen is reduced by 56%. As a non-limiting example, the expression of HTT protein and mRNA in the putamen is reduced by 57%. As a non-limiting example, the expression of HTT protein and mRNA in the putamen is reduced by 58%. As a non-limiting example, the expression of HTT protein and mRNA in the putamen is reduced by 59%. As a non-limiting example, the expression of HTT protein and mRNA in the putamen is reduced by 60%.

### Solo and Combination Therapy

In some embodiments, the present composition is administered as a solo therapeutic or combination therapeutics for the treatment of HD.

In some embodiments, the pharmaceutical composition of the present invention is used as a solo therapy. In other embodiments, the pharmaceutical composition of the present invention is used in combination therapy. The combination therapy may be in combination with one or more neuroprotective agents such as small molecule compounds, growth factors and hormones which have been tested for their neuroprotective effect on neuron degeneration.

The AAV particles encoding siRNA duplexes targeting the HTT gene may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the AAV particles encoding the nucleic acid sequence for the siRNA molecules of the present invention can be small molecule compounds which are antioxidants, anti-inflammatory agents, anti-apoptosis agents, calcium regulators, antiglutamatergic agents, structural protein inhibitors, compounds involved in muscle function, and compounds involved in metal ion regulation.

Compounds tested for treating HD which may be used in combination with the vectors described herein include, but are not limited to, dopamine-depleting agents (e.g., tetrabenazine for chorea), benzodiazepines (e.g., clonazepam for myoclonus, chorea, dystonia, rigidity, and/or spasticity), anticonvulsants (e.g., sodium valproate and levetiracetam for myoclonus), amino acid precursors of dopamine (e.g., levodopa for rigidity which is particularly associate with juvenile HD or young adult-onset parkinsonian phenotype), skeletal muscle relaxants (e.g., baclofen, tizanidine for rigidity and/or spasticity), inhibitors for acetycholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), agents to increase ATP/cellular energetics (e.g., creatine), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the AAV particles encoding the nucleic acid sequence for the siRNA molecules of the present invention for treating HD. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the AAV particles comprising modulatory polynucleotides encoding the siRNA duplex targeting the HTT gene may be co-administered with AAV particles expressing neurotrophic factors such as AAV-IGF-I (See e.g., Vincent et al., Neuromolecular medicine, 2004, 6, 79-85; the content of which is incorporated herein by reference in its entirety) and AAV-GDNF (See e.g., Wang et al., J Neurosci., 2002, 22, 6920-6928; the content of which is incorporated herein by reference in its entirety).

### Amyotrophic Lateral Sclerosis (ALS)

### Amyotrophic Lateral Sclerosis (ALS)

Amyotrophic lateral sclerosis (ALS), an adult-onset neurodegenerative disorder, is a progressive and fatal disease characterized by the selective death of motor neurons in the motor cortex, brainstem and spinal cord. The incidence of ALS is about 1.9 per 100,000. Patients diagnosed with ALS develop a progressive muscle phenotype characterized by spasticity, hyperreflexia or hyporeflexia, fasciculations, muscle atrophy and paralysis. These motor impairments are caused by the denervation of muscles due to the loss of motor neurons. The major pathological features of ALS include degeneration of the corticospinal tracts and extensive loss of lower motor neurons (LMNs) or anterior horn cells (Ghatak et al., J Neuropathol Exp Neurol., 1986, 45, 385-395), degeneration and loss of Betz cells and other pyramidal cells in the primary motor cortex (Udaka et al., Acta Neuropathol, 1986, 70, 289-295; Maekawa et al., Brain, 2004, 127, 1237-1251) and reactive gliosis in the motor cortex and spinal cord (Kawamata et al., Am J Pathol., 1992, 140, 691-707; and Schiffer et al., J Neurol Sci., 1996, 139, 27-33). ALS is usually fatal within 3 to 5 years after the diagnosis due to respiratory defects and/or inflammation (Rowland L P and Shneibder N A, N Engl. J. Med., 2001, 344, 1688-1700).

A cellular hallmark of ALS is the presence of proteinaceous, ubiquitinated, cytoplasmic inclusions in degenerating motor neurons and surrounding cells (e.g., astrocytes). Ubiquitinated inclusions (i.e., Lewy body-like inclusions or Skein-like inclusions) are the most common and specific type of inclusion in ALS and are found in LMNs of the spinal cord and brainstem, and in corticospinal upper motor neurons (UMNs) (Matsumoto et al., J Neurol Sci., 1993, 115, 208-213; and Sasak and Maruyama, Acta Neuropathol., 1994, 87, 578-585). A few proteins have been identified to be components of the inclusions, including ubiquitin, Cu/Zn superoxide dismutase 1 (SOD1), peripherin and Dorfin. Neurofilamentous inclusions are often found in hyaline conglomerate inclusions (HCIs) and axonal ‘spheroids’ in spinal cord motor neurons in ALS. Other types and less specific inclusions include Bunina bodies (cystatin C-containing inclusions) and Crescent shaped inclusions (SCIs) in upper layers of the cortex. Other neuropathological features seen in ALS include fragmentation of the Golgi apparatus, mitochondrial vacuolization and ultrastructural abnormalities of synaptic terminals (Fujita et al., Acta Neuropathol. 2002, 103, 243-247).

In addition, in frontotemporal dementia ALS (FTD-ALS) cortical atrophy (including the frontal and temporal lobes) is also observed, which may cause cognitive impairment in FTD-ALS patients.

ALS is a complex and multifactorial disease and multiple mechanisms hypothesized as responsible for ALS pathogenesis include, but are not limited to, dysfunction of protein degradation, glutamate excitotoxicity, mitochondrial dysfunction, apoptosis, oxidative stress, inflammation, protein misfolding and aggregation, aberrant RNA metabolism, and altered gene expression.

About 10%-15% of ALS cases have family history of the disease, and these patients are referred to as familial ALS (fALS) or inherited patients, commonly with a Mendelian dominant mode of inheritance and high penetrance. The remainder (approximately 85%-95%) is classified as sporadic ALS (sALS), as they are not associated with a documented family history, but instead are thought to be due to other risk factors including, but not limited to environmental factors, genetic polymorphisms, somatic mutations, and possibly gene-environmental interactions. In most cases, familial (or inherited) ALS is inherited as autosomal dominant disease, but pedigrees with autosomal recessive and X-linked inheritance and incomplete penetrance exist. Sporadic and familial forms are clinically indistinguishable suggesting a common pathogenesis. The precise cause of the selective death of motor neurons in ALS remains elusive. Progress in understanding the genetic factors in fALS may shed light on both forms of the disease.

Recently, an explosion to genetic causes of ALS has discovered mutations in more than 10 different genes that are known to cause fALS. The most common ones are found in the genes encoding Cu/Zn superoxide dismutase 1 (SOD1; ˜20%) (Rosen D R et al., Nature, 1993, 362, 59-62), fused in sarcoma/translated in liposarcoma (FUS/TLS; 1-5%) and TDP-43 (TARDBP; 1-5%). Recently, a hexanucleotide repeat expansion (GGGGCC)ₙ in the C9orF72 gene was identified as the most frequent cause of fALS (˜40%) in the Western population (reviewed by Renton et al., Nat. Neurosci., 2014, 17, 17-23). Other genes mutated in ALS include alsin (ALS2), senataxin (SETX), vesicle-associated membrane protein (VAPB), and angiogenin (ANG). fALS genes control different cellular mechanisms, suggesting that the pathogenesis of ALS is complicated and may be related to several different processes finally leading to motor neuron degeneration.

SOD1 is one of the three human superoxide dismutases identified and characterized in mammals: copper-zinc superoxide dismutase (Cu/ZnSOD or SOD1), manganese superoxide dismutase (MnSOD or SOD2), and extracellular superoxide dismutase (ECSOD or SOD3). SOD1 is a 32 kDa homodimer of a 153-residue polypeptide with one copper- and one zinc-binding site per subunit, which is encoded by the SOD1 gene (GeneBank access No.: NM_000454.4; SEQ ID NO: 1502) on human chromosome 21. SOD1 catalyzes the reaction of superoxide anion (O²⁻) into molecular oxygen (O₂) and hydrogen peroxide (H₂O₂) at a bound copper ion. The intracellular concentration of SOD1 is high (ranging from 10 to 100 μM), accounting for 1% of the total protein content in the central nervous system (CNS). The protein is localized not only in the cytoplasm but also in the nucleus, lysosomes, peroxisomes, and mitochondrial intermembrane spaces in eukaryotic cells (Lindenau J et al., Glia, 2000, 29, 25-34).

Mutations in the SOD1 gene are carried by 15-20% of fALS patients and by 1-2% of all ALS cases. Currently, at least 170 different mutations distributed throughout the 153-amino acid SOD1 polypeptide have been found to cause ALS, and an updated list can be found at the ALS online Genetic Database (ALSOD) (Wroe R et al., Amyotroph Lateral Scler., 2008, 9, 249-250). Table 46 lists some examples of mutations in SOD1 in ALS. These mutations are predominantly single amino acid substitutions (i.e. missense mutations) although deletions, insertions, and C-terminal truncations also occur. Different SOD1 mutations display different geographic distribution patterns. For instance, 40-50% of all Americans with ALS caused by SOD1 gene mutations have a particular mutation Ala4Val (or A4V). The A4V mutation is typically associated with more severe signs and symptoms and the survival period is typically 2-3 years. The I113T mutation is by far the most common mutation in the United Kingdom. The most prevalent mutation in Europe is D90A substitute and the survival period is usually greater than 10 years.

TABLE 46
Examples of SOD1 mutations in ALS

| Location | Mutations |
|---|---|
| Exon1 | Q22L; E21K, G; F20C; N19S; |
| (220 bp) | G16A, S; V14M, S; G12R; G10G, V, R; |
| | L8Q, V; V7E; C6G, F; V5L; A4T, V, S |
| Exon2 | T54R; E49K; H48R, Q; |
| (97 bp) | V47F, A; H46R; F45C; H43R; G41S, D; |
| | G37R; V29, insA |
| Exon3 | D76Y, V; G72S, C; L67R; |
| (70 bp) | P66A; N65S; S59I, S |
| Exon4 | D124G, V; |
| (118 bp) | V118L, InsAAAAC; L117V; T116T; |
| | R115G; G114A; I113T, F; I112M, T; |
| | G108V; L106V, F; S106L, delTCACTC; |
| | I104F; D101G, Y, H, N; E100G, K; I99V; |
| | V97L, M; D96N, V; A95T, V; |
| | G93S, V, A, C, R, D; D90V, A; A89T, V; |
| | T88delACTGCTGAC; V87A, M; |
| | N86I, S, D, K; G85R, S; L84V, F; H80R |
| Exon5 | I151T, S; I149T; V148I, G; |
| (461 bp) | G147D, R; C146R, stop; A145T, G; |
| | L144F, S; G141E, stop; A140A, G; |
| | N139D, K, H, N; G138E; T137R; |
| | S134N; E133V, delGAA, insTT; |
| | E132insTT; G127R, InsTGGG; |
| | L126S, delITT, stop; D126, delTT |

To investigate the mechanism of neuronal death associated with SOD1 gene defects, several rodent models of SOD1-linked ALS were developed in the art, which express the human SOD1 gene with different mutations, including missense mutations, small deletions or insertions. Non-limiting examples of ALS mouse models include SOD1^{G93A}, SOD1^{A4V}, SOD1^{G37R}, SOD1^{G85R}, SOD1^{90A}, SOD1^{L84V}, SOD1^{I113T}, SOD1^{H36R/H48Q}, SOD1^{G127X}, SOD1^{L126X} and SOD1^{L126delTT}. There are two transgenic rat models carrying two different human SOD1 mutations: SOD1^{H46R} and SOD1^{G93R}. These rodent ALS models can develop muscle weakness similar to human ALS patients and other pathogenic features that reflect several characteristics of the human disease, in particular, the selective death of spinal motor neurons, aggregation of protein inclusions in motor neurons and microglial activation. It is well known in the art that the transgenic rodents are good models of human SOD1-associated ALS disease and provide models for studying disease pathogenesis and developing disease treatment.

Studies in animal and cellular models showed that SOD1 pathogenic variants cause ALS by gain of function. That is to say, the superoxide dismutase enzyme gains new but harmful properties when altered by SOD1 mutations. For example, some SOD1 mutated variants in ALS increase oxidative stress (e.g., increased accumulation of toxic superoxide radicals) by disrupting the redox cycle. Other studies also indicate that some SOD1 mutated variants in ALS might acquire toxic properties that are independent of its normal physiological function (such as abnormal aggregation of misfolded SOD1 variants. In the aberrant redox chemistry model, mutant SOD1 is unstable and through aberrant chemistry interacts with nonconventional substrates causing overproduction of reactive oxygen species (ROS). In the protein toxicity model, unstable, misfolded SOD1 aggregates into cytoplasmic inclusion bodies, sequestering proteins crucial for cellular processes. These two hypotheses are not mutually exclusive. It has been shown that oxidation of selected histidine residues that bind metals in the active site mediates SOD1 aggregation.

The aggregated mutant SOD1 protein may also induce mitochondrial dysfunction (Vehvilainen P et al., Front Cell Neurosci., 2014, 8, 126), impairment of axonal transport, aberrant RNA metabolism, glial cell pathology and glutamate excitotoxicity. In some sporadic ALS cases, misfolded wild-type SOD1 protein is found in diseased motor neurons which forms a “toxic conformation” that is similar to that which is seen with familial ALS-linked SOD1 variants (Rotunno M S and Bosco D A, Front Cell Neurosci., 2013, 16, 7, 253). Such evidence suggests that ALS is a protein folding diseases analogous to other neurodegenerative diseases such as Alzheimer's disease and Parkinson's disease.

Currently, no curative treatments are available for patients suffering from ALS. The only FDA approved drug Riluzole, an inhibitor of glutamate release, has a moderate effect on ALS, only extending survival by 2-3 months if it is taken for 18 months. Unfortunately, patients taking riluzole do not experience any slowing in disease progression or improvement in muscle function. Therefore, riluzole does not present a cure, or even an effective treatment. Researchers continue to search for better therapeutic agents.

Therapeutic approaches that may prevent or ameliorate SOD1 aggregation have been tested previously. For example, arimoclomol, a hydroxylamine derivative, is a drug that targets heat shock proteins, which are cellular defense mechanisms against these aggregates. Studies demonstrated that treatment with arimoclomol improved muscle function in SOD1 mouse models. Other drugs that target one or more cellular defects in ALS may include AMPA antagonists such as talampanel, beta-lactam antibiotics, which may reduce glutamate-induced excitotoxicity to motor neurons; Bromocriptine that may inhibit oxidative induced motor neuron death (e.g. U.S. Patent publication No. 20110105517; the content of which is incorporated herein by reference in its entirety); 1,3-diphenylurea derivative or multikinase inhibitor which may reduce SOD1 gene expression (e.g., U.S. Patent Publication No. 20130225642; the content of which is incorporated herein by reference in its entirety); dopamine agonist pramipexole and its anantiomer dexpramipexole, which may ameliorate the oxidative response in mitochondria; nimesulide, which inhibits cyclooxygenase enzyme (e.g., U.S. Patent Publication No. 20060041022; the content of which is incorporated herein by reference in its entirety); drugs that act as free radical scavengers (e.g. U.S. Pat. No. 6,933,310 and PCT Patent Publication No.: WO2006075434; the content of each of which is incorporated herein by reference in their entirety).

Another approach to inhibit abnormal SOD1 protein aggregation is to silence/inhibit SOD1 gene expression in ALS. It has been reported that small interfering RNAs for specific gene silencing of the mutated allele are therapeutically beneficial for the treatment of fALS (e.g., Ralgh G S et al., Nat. Medicine, 2005, 11(4), 429-433; and Raoul C et al., Nat. Medicine, 2005, 11(4), 423-428; and Maxwell M M et al., PNAS, 2004, 101(9), 3178-3183; and Ding H et al., Chinese Medical J., 2011, 124(1), 106-110; and Scharz D S et al., Plos Genet., 2006, 2(9), e140; the content of each of which is incorporated herein by reference in their entirety).

Many other RNA therapeutic agents that target the SOD1 gene and modulate SOD1 expression in ALS are taught in the art. Such RNA based agents include antisense oligonucleotides and double stranded small interfering RNAs. See, e.g., Wang H et al., J Biol. Chem., 2008, 283(23), 15845-15852); U.S. Pat. Nos. 7,498,316; 7,632,938; 7,678,895; 7,951,784; 7,977,314; 8,183,219; 8,309,533 and 8, 586, 554; and U.S. Patent publication Nos. 2006/0229268 and 2011/0263680; the content of each of which is herein incorporated by reference in their entirety.

The present invention provides AAV particles comprising modulatory polynucleotides comprising sequences encoding siRNA molecules targeting the SOD1 gene and methods for their design and manufacture. The AAV particles comprising the nucleic acid sequence encoding the siRNA molecules of the present invention may increase the delivery of active agents into motor neurons. The siRNA duplexes or encoding dsRNA targeting the SOD1 gene may be able to inhibit SOD1 gene expression (e.g., mRNA level) significantly inside cells; therefore, ameliorating SOD1 expression induced stress inside the cells such as aggregation of protein and formation of inclusions, increased free radicals, mitochondrial dysfunction and RNA metabolism.

Such siRNA mediated SOD1 expression inhibition may be used for treating ALS. According to the present invention, methods for treating and/or ameliorating ALS in a patient comprises administering to the patient an effective amount of AAV particle comprising a nucleic acid sequence encoding the siRNA molecules of the present invention into cells. The administration of the AAV particle comprising such a nucleic acid sequence will encode the siRNA molecules which cause the inhibition/silence of SOD1 gene expression.

In one embodiment, the AAV particle comprising the modulatory polynucleotide, reduce the expression of mutant SOD1 in a subject. The reduction of mutant SOD1 can also reduce the formation of toxic aggregates which can cause mechanisms of toxicity such as, but not limited to, oxidative stress, mitochondrial dysfunction, impaired axonal transport, aberrant RNA metabolism, glial cell pathology and/or glutamate excitotoxicity.

In one embodiment, the vector, e.g., AAV particles, reduces the amount of SOD1 in a subject in need thereof and thus provides a therapeutic benefit as described herein.

### Methods of Treatment of ALS

Provided in the present invention are methods for introducing the AAV particles comprising modulatory polynucleotides comprising sequences comprising a nucleic acid sequence encoding the siRNA molecules of the present invention into cells, the method comprising introducing into said cells any of the vectors in an amount sufficient for degradation of target SOD1 mRNA to occur, thereby activating target-specific RNAi in the cells. In some aspects, the cells may be stem cells, neurons such as motor neurons, muscle cells and glial cells such as astrocytes.

Disclosed in the present invention are methods for treating ALS associated with abnormal SOD1 function in a subject in need of treatment. The method optionally comprises administering to the subject a therapeutically effective amount of a composition comprising at least AAV particles comprising modulatory polynucleotides comprising a nucleic acid sequence encoding the siRNA molecules of the present invention. As a non-limiting example, the siRNA molecules can silence SOD1 gene expression, inhibit SOD1 protein production, and reduce one or more symptoms of ALS in the subject such that ALS is therapeutically treated.

In some embodiments, the composition comprising the AAV particles comprising modulatory polynucleotides comprising a nucleic acid sequence encoding the siRNA molecules of the present invention is administered to the central nervous system of the subject. In other embodiments, the composition comprising the AAV particles comprising modulatory polynucleotides comprising a nucleic acid sequence encoding the siRNA molecules of the present invention is administered to the muscles of the subject

In particular, the AAV particles comprising modulatory polynucleotides comprising a nucleic acid sequence encoding the siRNA molecules of the present invention may be delivered into specific types of targeted cells, including motor neurons; glial cells including oligodendrocyte, astrocyte and microglia; and/or other cells surrounding neurons such as T cells. Studies in human ALS patients and animal SOD1 ALS models implicate glial cells as playing an early role in the dysfunction and death of motor neurons. Normal SOD1 in the surrounding, protective glial cells can prevent the motor neurons from dying even though mutant SOD1 is present in motor neurons (e.g., reviewed by Philips and Rothstein, Exp. Neurol., 2014, May 22. pii: S0014-4886(14)00157-5; the content of which is incorporated herein by reference in its entirety).

In some specific embodiments, the AAV particles comprising modulatory polynucleotides comprising a nucleic acid sequence encoding the siRNA molecules of the present invention may be used as a therapy for ALS.

In some embodiments, the present composition is administered as a solo therapeutics or combination therapeutics for the treatment of ALS.

The AAV particles comprising modulatory polynucleotides comprising a nucleic acid sequence encoding the siRNA molecules targeting the SOD1 gene may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the AAV particles comprising modulatory polynucleotides comprising a nucleic acid sequence encoding the siRNA molecules of the present invention can be small molecule compounds which are antioxidants, anti-inflammatory agents, anti-apoptosis agents, calcium regulators, antiglutamatergic agents, structural protein inhibitors, and compounds involved in metal ion regulation.

Compounds tested for treating ALS which may be used in combination with the vectors described herein include, but are not limited to, antiglutamatergic agents: Riluzole, Topiramate, Talampanel, Lamotrigine, Dextromethorphan, Gabapentin and AMPA antagonist; Anti-apoptosis agents: Minocycline, Sodium phenylbutyrate and Arimoclomol; Anti-inflammatory agent: ganglioside, Celecoxib, Cyclosporine, Azathioprine, Cyclophosphamide, Plasmaphoresis, Glatiramer acetate and thalidomide; Ceftriaxone (Berry et al., Plos One, 2013, 8(4)); Beat-lactam antibiotics; Pramipexole (a dopamine agonist) (Wang et al., Amyotrophic Lateral Scler., 2008, 9(1), 50-58); Nimesulide in U.S. Patent Publication No. 20060074991; Diazoxide disclosed in U.S. Patent Publication No. 20130143873); pyrazolone derivatives disclosed in US Patent Publication No. 20080161378; free radical scavengers that inhibit oxidative stress-induced cell death, such as bromocriptine Patent Publication No. 20110105517); phenyl carbamate compounds discussed in PCT Patent Publication No. 2013100571; neuroprotective compounds disclosed in U.S. Pat. Nos. 6,933,310 and 8,399,514 and US Patent Publication Nos. 20110237907 and 20140038927; and glycopeptides taught in U.S. Patent Publication No. 20070185012; the content of each of which is incorporated herein by reference in their entirety.

Therapeutic agents that may be used in combination therapy with the AAV particles comprising modulatory polynucleotides comprising a nucleic acid sequence encoding the siRNA molecules of the present invention may be hormones or variants that can protect neuronal loss, such as adrenocorticotropic hormone (ACTH) or fragments thereof (e.g., U.S. Patent Publication No. 20130259875); Estrogen (e.g., U.S. Pat. Nos. 6,334,998 and 6,592,845); the content of each of which is incorporated herein by reference in their entirety.

Neurotrophic factors may be used in combination therapy with the AAV particles comprising modulatory polynucleotides comprising a nucleic acid sequence encoding the siRNA molecules of the present invention for treating ALS. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the vector, e.g., AAV particle, encoding the nucleic acid sequence for the at least one siRNA duplex targeting the SOD1 gene may be co-administered with AAV particles expressing neurotrophic factors such as AAV-IGF-I (Vincent et al., Neuromolecular medicine, 2004, 6, 79-85; the content of which is incorporated herein by reference in its entirety) and AAV-GDNF (Wang et al., J Neurosci., 2002, 22, 6920-6928; the content of which is incorporated herein by reference in its entirety).

In some embodiments, the composition of the present invention for treating ALS is administered to the subject in need intravenously, intramuscularly, subcutaneously, intraperitoneally, intrathecally and/or intraventricularly, allowing the siRNA molecules or vectors comprising the siRNA molecules to pass through one or both the blood-brain barrier and the blood spinal cord barrier. In some aspects, the method includes administering (e.g., intraventricularly administering and/or intrathecally administering) directly to the central nervous system (CNS) of a subject (using, e.g., an infusion pump and/or a delivery scaffold) a therapeutically effective amount of a composition comprising AAV particles comprising modulatory polynucleotides comprising a nucleic acid sequence encoding the siRNA molecules of the present invention. The vectors may be used to silence or suppress SOD1 gene expression, and/or reducing one or more symptoms of ALS in the subject such that ALS is therapeutically treated.

In certain aspects, the symptoms of ALS include, but are not limited to, motor neuron degeneration, muscle weakness, muscle atrophy, the stiffness of muscle, difficulty in breathing, slurred speech, fasciculation development, frontotemporal dementia and/or premature death are improved in the subject treated. In other aspects, the composition of the present invention is applied to one or both of the brain and the spinal cord. In other aspects, one or both of muscle coordination and muscle function are improved. In other aspects, the survival of the subject is prolonged.

In one embodiment, administration of the AAV particles comprising modulatory polynucleotides comprising a nucleic acid sequence encoding the siRNA molecules of the present invention, to a subject may lower mutant SOD1 in the CNS of a subject. In another embodiment, administration of the AAV particles, to a subject may lower wild-type SOD1 in the CNS of a subject. In yet another embodiment, administration of the AAV particles, to a subject may lower both mutant SOD1 and wild-type SOD1 in the CNS of a subject. The mutant and/or wild-type SOD1 may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in the CNS, a region of the CNS, or a specific cell of the CNS of a subject. As a non-limiting example, the AAV particles may lower the expression of wild-type SOD1 by at least 50% in the motor neurons (e.g., ventral horn motor neurons) and/or astrocytes. As another non-limiting example, the AAV particles may lower the expression of mutant SOD1 by at least 50% in the motor neurons (e.g., ventral horn motor neurons) and/or astrocytes. As yet another non-limiting example, the AAV particles may lower the expression of wild-type SOD1 and mutant SOD1 by at least 50% in the motor neurons (e.g., ventral horn motor neurons) and/or astrocytes.

In one embodiment, administration of the AAV particles, to a subject will reduce the expression of mutant and/or wild-type SOD1 in the spinal cord and the reduction of expression of the mutant and/or wild-type SOD1 will reduce the effects of ALS in a subject.

In one embodiment, the AAV particles may be administered to a subject who is in the early stages of ALS. Early stage symptoms include, but are not limited to, muscles which are weak and soft or stiff, tight and spastic, cramping and twitching (fasciculations) of muscles, loss of muscle bulk (atrophy), fatigue, poor balance, slurred words, weak grip, and/or tripping when walking. The symptoms may be limited to a single body region or a mild symptom may affect more than one region. As a non-limiting example, administration of the AAV particles may reduce the severity and/or occurrence of the symptoms of ALS.

In one embodiment, the AAV particles may be administered to a subject who is in the middle stages of ALS. The middle stage of ALS includes, but is not limited to, more widespread muscle symptoms as compared to the early stage, some muscles are paralyzed while others are weakened or unaffected, continued muscle twitchings (fasciculations), unused muscles may cause contractures where the joints become rigid, painful and sometimes deformed, weakness in swallowing muscles may cause choking and greater difficulty eating and managing saliva, weakness in breathing muscles can cause respiratory insufficiency which can be prominent when lying down, and/or a subject may have bouts of uncontrolled and inappropriate laughing or crying (pseudobulbar affect). As a non-limiting example, administration of the AAV particles may reduce the severity and/or occurrence of the symptoms of ALS.

In one embodiment, the AAV particles may be administered to a subject who is in the late stages of ALS. The late stage of ALS includes, but is not limited to, voluntary muscles which are mostly paralyzed, the muscles that help move air in and out of the lungs are severely compromised, mobility is extremely limited, poor respiration may cause fatigue, fuzzy thinking, headaches and susceptibility to infection or diseases (e.g., pneumonia), speech is difficult and eating or drinking by mouth may not be possible.

In one embodiment, the AAV particles may be used to treat a subject with ALS who has a C9orf72 mutation.

In one embodiment, the AAV particles may be used to treat a subject with ALS who has TDP-43 mutations.

In one embodiment, the AAV particles may be used to treat a subject with ALS who has FUS mutations.

In one embodiment, the AAV particle of the present invention comprises an AAVrh10 capsid and a self-complementary AAV viral genome comprising an H1 promoter, a stuffer sequence originating from a pLKO.1 lentiviral vector and a SOD1 targeting payload.

In one embodiment, the AAV particle of the present invention comprises an AAV2 capsid and a self-complementary AAV viral genome.

In one embodiment, the AAV particle of the present invention comprises an AAV2 capsid and a self-complementary AAV viral genome comprising an H1 promoter, a stuffer sequence originating from a pLKO.1 lentiviral vector and a SOD1 targeting payload.

### V. Definitions

Unless stated otherwise, the following terms and phrases have the meanings described below. The definitions are not meant to be limiting in nature and serve to provide a clearer understanding of certain aspects of the present invention.

As used herein, the term “nucleic acid”, “polynucleotide” and ‘oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the other siRNA strand.

As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity.

As used herein, the term “substantially complementary” means that the siRNA has a sequence (e.g., in the antisense strand) which is sufficient to bind the desired target mRNA, and to trigger the RNA silencing of the target mRNA.

As used herein, “targeting” means the process of design and selection of nucleic acid sequence that will hybridize to a target nucleic acid and induce a desired effect.

The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

As used herein, the term “vector” means any molecule or moiety which transports, transduces or otherwise acts as a carrier of a heterologous molecule such as the siRNA molecule of the invention. A “viral genome” or “vector genome” or “viral vector” refers to a sequence which comprises one or more polynucleotide regions encoding or comprising a molecule of interest, e.g., a transgene, a polynucleotide encoding a polypeptide or multi-polypeptide or a modulatory nucleic acid such as small interfering RNA (siRNA). Viral genomes are commonly used to deliver genetic materials into cells. Viral genomes are often modified for specific applications. Types of viral genome sequence include retroviral viral genome sequences, lentiviral viral genome sequences, adenoviral viral genome sequences and adeno-associated viral genome sequences.

The term “adeno-associated virus” or “AAV” as used herein refers to any vector which comprises or derives from components of an adeno-associated vector and is suitable to infect mammalian cells, preferably human cells. The term AAV vector typically designates an AAV type viral particle or virion comprising a payload. The AAV vector may be derived from various serotypes, including combinations of serotypes (i.e., “pseudotyped” AAV) or from various genomes (e.g., single stranded or self-complementary). In addition, the AAV vector may be replication defective and/or targeted.

As used herein, the phrase “inhibit expression of a gene” means to cause a reduction in the amount of an expression product of the gene. The expression product can be a RNA molecule transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “modified” refers to a changed state or structure of a molecule of the invention. Molecules may be modified in many ways including chemically, structurally, and functionally.

As used herein, the term “synthetic” means produced, prepared, and/or manufactured by the hand of man. Synthesis of polynucleotides or polypeptides or other molecules of the present invention may be chemical or enzymatic.

As used herein, the term “transfection” refers to methods to introduce exogenous nucleic acids into a cell. Methods of transfection include, but are not limited to, chemical methods, physical treatments and cationic lipids or mixtures. The list of agents that can be transfected into a cell is large and includes, but is not limited to, siRNA, sense and/or anti-sense sequences, DNA encoding one or more genes and organized into an expression plasmid, proteins, protein fragments, and more.

As used herein, “off target” refers to any unintended effect on any one or more target, gene, or cellular transcript.

As used herein, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “effective amount” of an agent is that amount sufficient to effect beneficial or desired results, for example, clinical results, and, as such, an “effective amount” depends upon the context in which it is being applied. For example, in the context of administering an agent that treats HD, an effective amount of an agent is, for example, an amount sufficient to achieve treatment, as defined herein, of HD, as compared to the response obtained without administration of the agent. For example, in the context of administering an agent that treats ALS, an effective amount of an agent is, for example, an amount sufficient to achieve treatment, as defined herein, of ALS, as compared to the response obtained without administration of the agent.

As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates such as chimpanzees and other apes and monkey species, and humans) and/or plants.

As used herein, the term “preventing” or “prevention” refers to delaying or forestalling the onset, development or progression of a condition or disease for a period of time, including weeks, months, or years.

The term “treatment” or “treating,” as used herein, refers to the application of one or more specific procedures used for the cure or amelioration of a disease. In certain embodiments, the specific procedure is the administration of one or more pharmaceutical agents. In the context of the present invention, the specific procedure is the administration of one or more siRNA molecules.

As used herein, the term “amelioration” or “ameliorating” refers to a lessening of severity of at least one indicator of a condition or disease. For example, in the context of neurodegeneration disorder, amelioration includes the reduction of neuron loss.

As used herein, the term “administering” refers to providing a pharmaceutical agent or composition to a subject.

As used herein, the term “neurodegeneration” refers to a pathologic state which results in neural cell death. A large number of neurological disorders share neurodegeneration as a common pathological state. For example, Alzheimer's disease, Parkinson's disease, Huntington's disease, and amyotrophic lateral sclerosis (ALS) all cause chronic neurodegeneration, which is characterized by a slow, progressive neural cell death over a period of several years, whereas acute neurodegeneration is characterized by a sudden onset of neural cell death as a result of ischemia, such as stroke, or trauma, such as traumatic brain injury, or as a result of axonal transection by demyelination or trauma caused, for example, by spinal cord injury or multiple sclerosis. In some neurological disorders, mainly one type of neuronal cell is degenerative, for example, medium spiny neuron degeneration in early HD.

### VI. Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

### VII. Examples

### Example 1. AAV-miRNA Expression Vectors

The constructs comprising the pri-miRNA cassettes containing guide strands targeting HTT and passenger strands were engineered into AAV-miRNA expression vectors (either ss or sc). The AAV-miRNA expression vector construct from ITR to ITR, recited 5′ to 3′, comprises an ITR (mutant or wild-type), a promoter comprising either a CMV (which includes an SV40 intron), U6, H1, CBA (which includes a CMVie enhancer, a CB promoter and an SV40 intron) or CAG promoter (which includes a CMVie enhancer, a CB promoter and a rabbit betaglobin intron), the pri-miRNA cassette, a rabbit globin polyA or human growth hormone and wild type ITR. In vitro and in vivo studies are performed to evaluate the pharmacological activity of the AAV-miRNA expression vectors.

### Example 2. In Vivo Studies of AAV-miRNA

### A. In Vivo Studies of Efficacy

Based on HTT suppression in YAC128 mice, guide to passenger ratio, and precision of 5′ end processing, selected AAV-miRNA expression vectors are packaged in AAV1 (either as ss or sc) with a CBA promoter (AAV1.CBA.iHtt), formulated in phosphate buffered saline (PBS) with 0.001% F-68 and administered to YAC128 mice to assess efficacy. AAV1 vectors are administered to YAC128 mice 7-12 weeks of age via bilateral intrastriatal infusion at a dose of approximately 1E10 to 3E10 vg in 5 uL over 10 minutes per hemisphere. A control group is treated with vehicle (PBS with 0.001% F-68). Following test article administration, behavioral tests including rotarod and Porsolt swim tests are performed at pre-determined time intervals, to assess efficacy. At a pre-determined day post-dosing, animals are euthanized, and striatum tissue punches are collected and snap-frozen. Tissue samples are homogenized and the total RNA is purified. The relative expression of HTT is determined by qRT-PCR. Housekeeping genes for normalization included mouse XPNPEP1. HTT is normalized to housekeeping gene expression, and then further normalized to the vehicle group. Samples are also used to quantify HTT protein.

### B. In Vivo Study in NHP of HTT Suppression, Guide to Passenger Ratio and 5′ End Precision of Processing

Based on HTT suppression in YAC128 mice, guide to passenger ratio, and precision of 5′ end processing, selected AAV-miRNA expression vectors are packaged in AAV1 with a CBA promoter (AAV1.CBA.iHtt), formulated in phosphate buffered saline (PBS) with 0.001% F-68 and administered to non-human primates by intraparenchymal brain infusion. A control group is treated with vehicle (PBS with 0.001% F-68). The relative expression of HTT mRNA, guide to passenger ratio, and the precision of 5′ end processing is determined in various tissue samples at a pre-determined time post-dosing.

### Example 3. Activity of Polycistronic Constructs in HEK293T and HeLa Cells

The polycistronic miRNA expression vectors encoding VOYHTmiR-104.016 (SEQ ID NO: 1589) and VOYHTmiR-127.579 (SEQ ID NO: 1599) were packaged in AAV2, and infected into HEK293T cells and HeLa cells. For HEK293T, the cells were plated into 96-well plates (2.5E4 cells/well in 100 ul cell culture medium) and infected with polycistronic miRNA expression vectors. The HeLa cells were plated into 96-well plates (1E4 cells/well in 100 ul cell culture medium). 24 hours after infection, the cells were harvested for immediate cell lysis and measurement of luciferase activity or isolation of RNA for qRT-PCR.

### A. Activity of Polycistronic Constructs (125 pM and 250 pM)

The relative activity (relative luciferase) of the polycistronic constructs 48 hours after transfection at 125 pM and 250 pM was determined by luciferase activity for the HEK293T and HeLa cells. The relative activity was obtained by normalizing the renilla luciferase level to the interal control firefly luciferase level as determined by duo-luciferase assay.

The RLU for the polycistronic constructs and the description of the constructs tested are shown in Table 47. In Table 47, two modulatory polynucleotides were tested in each vector and the modulatory polynucleotides were in tandem. In the table, the vector encodes the A modulatory polynucleotide before the B modulatory polynucleotide.

For the HEK293T cells, the control had a RLU of 1 at 125 pM and 1.11 at 250 pM. The construct encoding one VOYHTmiR-104.016 modulatory polynucleotide (SEQ ID NO: 1589) transfected at 125 pM provided a RLU of 0.13 and at 250 pM provided a RLU of 0.14. The construct encoding the VOYHTmiR-127.579 modulatory polynucleotide (SEQ ID NO: 1599) transfected at 125 pM provided a RLU of 0.14 and at 250 pM provided a RLU of 0.14. When two vectors each encoding one of the two modulatory polynucleotides (VOYHTmiR-104.016 (SEQ ID NO: 1589]) and VOYHTmiR-127.579 (SEQ ID NO: 1599)) were transfected at the same time at 125 pM each, a RLU of 0.06 was seen.

For the HeLa cells, the control had a RLU of 1 at 125 pM and 0.99 at 250 pM. The construct encoding the VOYHTmiR-104.016 modulatory polynucleotide (SEQ ID NO: 1589) transfected at 125 pM provided a RLU of 0.26 and at 250 pM provided a RLU of 0.27. The construct encoding the VOYHTmiR-127.579 modulatory polynucleotide (SEQ ID NO: 1599) transfected at 125 pM provided a RLU of 0.20 and at 250 pM provided a RLU of 0.12. When two constructs each encoding one of the two modulatory polynucleotides (VOYHTmiR-104.016 (SEQ ID NO: 1589) and VOYHTmiR-127.579 (SEQ ID NO: 1599)) were transfected at the same time at 125 pM each, a RLU of 0.22 was seen.

TABLE 47
Polycistronic activity

| Modulatory | Modulatory | | RLU | | | |
|---|---|---|---|---|---|---|
| Polynucleotide | Polynucleotide | Sequence | HEK293T | | HeLa | |
| Name | SEQ ID | Name | 125 pM | 250 pM | 125 pM | 250 pM |
| A: VOYHTmiR-104.016 | A: 1589 | VOYPC13 | 0.13 | 0.17 | 0.23 | 0.26 |
| B: VOYHTmiR-104.016 | B: 1589 | | | | | |
| A: VOYHTmiR-127.579 | A: 1599 | VOYPC14 | 0.05 | 0.06 | 0.08 | 0.09 |
| B: VOYHTmiR-104.016 | B: 1589 | | | | | |
| A: VOYHTmiR-104.016 | A: 1589 | VOYPC15 | 0.05 | 0.06 | 0.16 | 0.17 |
| B: VOYHTmiR-127.579 | B: 1599 | | | | | |
| A: VOYHTmiR-127.579 | A: 1599 | VOYPC16 | 0.13 | 0.14 | 0.18 | 0.17 |
| B: VOYHTmiR-127.579 | B: 1599 | | | | | |

The vectors encoding both the VOYHTmiR-104.016 and VOYHTmiR-127.579 in tandem showed the best activity as compared to the controls.

### B. Activity of Polycistronic Constructs (62.5 pM and 125 pM) and Length of Vector

The relative activity (relative luciferase) of the polycistronic constructs with and without filler DNA (to make the total DNA content the same in each condition) 40 hours after transfection at 62.5 pM and 125 pM was determined by Duo-Luciferase assay for HeLa cells. The relative activity was obtained by normalizing the renilla luciferase level to the internal control firefly luciferase level as determined by duo-luciferase assay. The RLU for the polycistronic constructs and the description of the constructs tested are shown in Table 48. In Table 48, two modulatory polynucleotides were tested in each construct and the modulatory polynucleotides were in tandem. In the table, the construct encodes the A modulatory polynucleotide before the B modulatory polynucleotide.

For constructs with filler DNA, the control had a RLU of 1 at 62.5 pM and 125 pM. The constructs encoding the VOYHTmiR-104.016 modulatory polynucleotide (SEQ ID NO: 1589) transfected at 62.5 pM provided a RLU of 0.45 and at 125 pM provided a RLU of 0.31. The constructs encoding the VOYHTmiR-127.579 modulatory polynucleotide (SEQ ID NO: 1599) transfected at 62.5 pM provided a RLU of 0.25 and at 125 pM provided a RLU of 0.20. When two constructs each encoding one of the two modulatory polynucleotides (VOYHTmiR-104.016 (SEQ ID NO: 1589) and VOYHTmiR-127.579 (SEQ ID NO: 1599)) were transfected at the same time at 62.5 pM each, a RLU of 0.26 was seen.

For constructs without filler DNA, the control had a RLU of 1 at 62.5 pM and 125 pM. The constructs encoding the VOYHTmiR-104.016 modulatory polynucleotide (SEQ ID NO: 1589) transfected at 62.5 pM provided a RLU of 0.31 and at 125 pM provided a RLU of 0.24. The constructs encoding the VOYHTmiR-127.579 modulatory polynucleotide (SEQ ID NO: 1599) transfected at 62.5 pM provided a RLU of 0.29 and at 125 pM provided a RLU of 0.24. When two constructs each encoding one of the two modulatory polynucleotides (VOYHTmiR-104.016 (SEQ ID NO: 1589) and VOYHTmiR-127.579 (SEQ ID NO: 1599)) were transfected at the same time at 62.5 pM each, a RLU of 0.23 was seen.

TABLE 48
Polycistronic activity

| Modulatory | Modulatory | | RLU | | | |
|---|---|---|---|---|---|---|
| Polynucleotide | Polynucleotide | Sequence | With Filler DNA | | Without Filler DNA | |
| Name | SEQ ID | Name | 62.5 pM | 125 pM | 62.5 pM | 125 pM |
| A: VOYHTmiR-104.016 | A: 1589 | VOYPC13 | 0.41 | 0.38 | 0.36 | 0.37 |
| B: VOYHTmiR-104.016 | B: 1589 | | | | | |
| A: VOYHTmiR-127.579 | A: 1599 | VOYPC14 | 0.17 | 0.15 | 0.16 | 0.15 |
| B: VOYHTmiR-104.016 | B: 1589 | | | | | |
| A: VOYHTmiR-104.016 | A: 1589 | VOYPC15 | 0.36 | 0.24 | 0.35 | 0.23 |
| B: VOYHTmiR-127.579 | B: 1599 | | | | | |
| A: VOYHTmiR-127.579 | A: 1599 | VOYPC16 | 0.30 | 0.23 | 0.29 | 0.23 |
| B: VOYHTmiR-127.579 | B: 1599 | | | | | |

Both the higher and lower dose of the constructs showed similar expression with and without the filler DNA. The constructs with the VOYHTmiR-127.579 and VOYHTmiR-104.016 modulatory polynucleotides in tandem showed the lowest RLU for both transfection conditions.

C. HTT Suppression after Transfection with Polycistronic Constructs

The relative expression of HTT mRNA 48 hours after transfection at a 125 pM or 250 pM was determined by qRT-PCR for HeLa. The relative HTT mRNA expression was obtained by normalizing the HTT mRNA level to the housekeeping gene mRNA level as determined by qRT-PCR; this normalized HTT mRNA level was then expressed relative to the normalized HTT mRNA level in control treated cells. The results for the polycistronic constructs and the description of the constructs tested are shown in Table 49. In Table 49, two modulatory polynucleotides were tested in each construct and the modulatory polynucleotides were in tandem. In the table, the construct encodes the A modulatory polynucleotide before the B modulatory polynucleotide.

The constructs encoding the VOYHTmiR-104.016 modulatory polynucleotide (SEQ ID NO: 1589) transfected at 125 pM provided a relative Htt mRNA level (normalized to control) of 50% and at 250 pM provided a relative Htt mRNA level (normalized to control) of 61%. The constructs encoding the VOYHTmiR-127.579 modulatory polynucleotide (SEQ ID NO: 1599) transfected at 125 pM provided a relative Htt mRNA level (normalized to control) of 52% and at 250 pM provided a relative Htt mRNA level (normalized to control) of 56%. When two constructs each encoding one of the two modulatory polynucleotides (VOYHTmiR-104.016 (SEQ ID NO: 1589) and VOYHTmiR-127.579 (SEQ ID NO: 1599)) were transfected at the same time at 125 pM each, a relative Htt mRNA level (normalized to control) of 49% was seen.

TABLE 49
Knock-Down of HTT

| | | | Relative HTT | |
|---|---|---|---|---|
| | | | mRNA Level (%) | |
| | Modulatory | | (normalized | |
| | polynu- | | to Control) | |
| Modulatory Polynu- | cleotide | Sequence | HeLa | |
| cleotide Name | SEQ ID | Name | 125 pM | 250 pM |
| A: VOYHTmiR-104.016 | A: 1589 | VOYPC13 | 43 | 50 |
| B: VOYHTmiR-104.016 | B: 1589 | | | |
| A: VOYHTmiR-127.579 | A: 1599 | VOYPC14 | 43 | 36 |
| B: VOYHTmiR-104.016 | B: 1589 | | | |
| A: VOYHTmiR-104.016 | A: 1589 | VOYPC15 | 49 | 50 |
| B: VOYHTmiR-127.579 | B: 1599 | | | |
| A: VOYHTmiR-127.579 | A: 1599 | VOYPC16 | 55 | 50 |
| B: VOYHTmiR-127.579 | B: 1599 | | | |

The constructs encoding both the VOYHTmiR-104.016 and VOYHTmiR-127.579 in tandem showed the best activity as compared to the controls for both transfection conditions.

D. HTT Suppression after Infection at MOI of 1E4 and 1E5 vg/cell

The relative expression of HTT mRNA 24 hours after infection at a MOI of 1E4 or 1E5 vg/cell was determined by qRT-PCR for HEK293T and HeLa cells. The relative HTT mRNA expression was obtained by normalizing the HTT mRNA level to the housekeeping gene mRNA level as determined by qRT-PCR; this normalized HTT mRNA level was then expressed relative to the normalized HTT mRNA level in mCherry-treated cells. The results are shown in Tables 50 and 51.

TABLE 50
Knock-Down of HTT

| | | Relative HTT | | | |
|---|---|---|---|---|---|
| | | mRNA Level (%) | | | |
| Modulatory | Modulatory | (normalized to Control) | | | |
| Polynucleotide | polynucleotide | HEK293T | | HeLa | |
| Name | SEQ ID | 1E4 | 1E5 | 1E4 | 1E5 |
| VOYHTmiR-104.016 | 1589 | 42 | 28 | 75 | 47 |
| VOYHTmiR-127.579 | 1599 | 44 | 29 | 67 | 45 |
| Construct 1: | Construct 1: | 35 | 27 | 67 | 46 |
| VOYHTmiR-104.016 | 1589 | | | | |
| Construct 2: | Construct 2: | | | | |
| VOYHTmiR-127.579 | 1599 | | | | |
| Untreated | — | 86 | 91 | 67 | 96 |

TABLE 51
Knock-Down of HTT

| | | | Relative HTT | | | |
|---|---|---|---|---|---|---|
| | | | mRNA Level (%) | | | |
| Modulatory | Modulatory | | (normalized to Control) | | | |
| Polynucleotide | polynucleotide | Sequence | HEK293T | | HeLa | |
| Name | SEQ ID | Name | 1E4 | 1E5 | 1E4 | 1E5 |
| A: VOYHTmiR-104.016 | A: 1589 | VOYPC13 | 48 | 27 | 47 | 46 |
| B: VOYHTmiR-104.016 | B: 1589 | | | | | |
| A: VOYHTmiR-127.579 | A: 1599 | VOYPC14 | 32 | 22 | 46 | 28 |
| B: VOYHTmiR-104.016 | B: 1589 | | | | | |
| A: VOYHTmiR-104.016 | A: 1589 | VOYPC15 | 30 | 23 | 60 | 26 |
| B: VOYHTmiR-127.579 | B: 1599 | | | | | |
| A: VOYHTmiR-127.579 | A: 1599 | VOYPC16 | 39 | 28 | 67 | 34 |
| B: VOYHTmiR-127.579 | B: 1599 | | | | | |

The vectors encoding both the VOYHTmiR-104.016 and VOYHTmiR-127.579 in tandem showed the best activity as compared to the controls for both infection levels in both cell types.

### E. Activity of Polycistronic Constructs (62.5 pM and 125 pM)

The relative activity (relative luciferase) of the polycistronic constructs 48 hours after transfection at 62.5 pM and 125 pM was determined by duo-luciferase assay for the HEK293T and HeLa cells. The relative activity was obtained by normalizing the renilla luciferase level to the internal control firefly luciferase level as determined by duo-luciferase assay. The RLU for the polycistronic constructs and the description of the constructs tested are shown in Tables 52-53. In Table 53, two, three or four modulatory polynucleotides were tested in each construct and the modulatory polynucleotides were in tandem. For example, if there are two modulatory polynucleotides, the construct encodes the A modulatory polynucleotide before the B modulatory polynucleotide.

TABLE 52
Knock-Down of HTT

| | | RLU | | | |
|---|---|---|---|---|---|
| Modulatory | Modulatory | HEK293T | | HEK293T | |
| Polynucleotide | polynucleotide | 62.5 | 62.5 | 62.5 | 62.5 |
| Name | SEQ ID | pM | pM | pM | pM |
| VOYHTmiR-104.016 | 1589 | 0.24 | 0.2 | 0.59 | 0.3 |
| VOYHTmiR-127.579 | 1599 | 0.31 | 0.23 | 0.84 | 0.27 |
| Construct 1: | Construct 1: | 0.1 | 0.11 | 0.25 | 0.2 |
| VOYHTmiR-104.016 | 1589 | | | | |
| Construct 2: | Construct 2: | | | | |
| VOYHTmiR-127.579 | 1599 | | | | |
| Untreated | — | 0.24 | 0.2 | 0.59 | 0.3 |

TABLE 53
Polycistronic activity

| Modulatory | Modulatory | | RLU | | | |
|---|---|---|---|---|---|---|
| Polynucleotide | polynucleotide | Sequence | 293T | | HeLa | |
| Name | SEQ ID | Name | 62.5 pM | 125 pM | 62.5 pM | 125 pM |
| A: VOYHTmiR-127.579 | A: 1599 | VOYPC14 | 0.11 | 0.09 | 0.26 | 0.11 |
| B: VOYHTmiR-104.016 | B: 1589 | | | | | |
| A: VOYHTmiR-127.579 | A: 1599 | VOYPC35 | 0.11 | 0.11 | 0.28 | 0.28 |
| B: VOYHTmiR-104.016 | B: 1589 | | | | | |
| C: VOYHTmiR-104.016 | C: 1589 | | | | | |
| A: VOYHTmiR-127.579 | A: 1599 | VOYPC36 | 0.07 | 0.07 | 0.16 | 0.10 |
| B: VOYHTmiR-104.016 | B: 1589 | | | | | |
| C: VOYHTmiR-127.579 | C: 1599 | | | | | |
| A: VOYHTmiR-127.579 | A: 1599 | VOYPC51 | 0.08 | 0.07 | 0.18 | 0.12 |
| B: VOYHTmiR-104.016 | B: 1589 | | | | | |
| C: VOYHTmiR-127.579 | C: 1599 | | | | | |
| D: VOYHTmiR-104.016 | D: 1589 | | | | | |
| A: VOYHTmiR-127.579 | A: 1599 | VOYPC52 | 0.07 | 0.07 | 0.16 | 0.10 |
| B: VOYHTmiR-104.016 | B: 1589 | | | | | |
| C: VOYHTmiR-104.016 | C: 1589 | | | | | |
| D: VOYHTmiR-127.579 | D: 1599 | | | | | |

The constructs encoding more than two modulatory polynucleotides gave the lowest RLU values for both transfection conditions in both cell types.

### Example 4. Activity of Polycistronic Constructs in HEK293T and HeLa Cells

The polycistronic miRNA expression constructs encoding VOYHTmiR-104.579 (SEQ ID NO: 1595) and VOYHTmiR-127.016 (SEQ ID NO: 1593) were packaged in scAAV2, and infected into HEK293T cells and HeLa cells. For HEK293T, the cells were plated into 96-well plates (2.5E4 cells/well in 100 ul cell culture medium) and infected with polycistronic miRNA expression vectors. The HeLa cells were plated into 96-well plates (1E4 cells/well in 100 ul cell culture medium). 24 hours after infection, the cells were harvested for immediate cell lysis and measurement of luciferase activity or isolation for qRT-PCR.

### A. Activity of Polycistronic Constructs (62.5 pM and 125 pM)

The relative activity (relative luciferase) of the polycistronic constructs 48 hours after transfection at 62.5 pM and 125 pM was determined by qRT-PCR for HEK293T and HeLa cells. The relative activity was obtained by normalizing the renilla luciferase level to the internal control firefly luciferase level as determined by duo-luciferase assay. The RLU for the polycistronic constructs and the description of the constructs tested are shown in Tables 54-55. In Table 55, two modulatory polynucleotides were tested in each construct and the modulatory polynucleotides were in tandem. In the table, the construct encodes the A modulatory polynucleotide before the B modulatory polynucleotide.

TABLE 54
Knock-Down of HTT

| | | RLU | | | |
|---|---|---|---|---|---|
| Modulatory | Modulatory | HEK293T | | HeLa | |
| Polynucleotide | polynucleotide | 62.5 | 125 | 62.5 | 125 |
| Name | SEQ ID | pM | pM | pM | pM |
| VOYHTmiR-104.579 | 1595 | 0.24 | 0.13 | 0.51 | 0.25 |
| VOYHTmiR-127.016 | 1593 | 0.33 | 0.16 | 0.23 | 0.22 |
| Construct 1: | Construct 1: | 0.07 | 0.06 | 0.08 | 0.12 |
| VOYHTmiR-104.579 | 1595 | | | | |
| Construct 2: | Construct 2: | | | | |
| VOYHTmiR-127.016 | 1593 | | | | |

TABLE 55
Polycistronic activity

| Modulatory | Modulatory | | RLU | | | |
|---|---|---|---|---|---|---|
| Polynucleotide | polynucleotide | Sequence | HEK293T | | HeLa | |
| Name | SEQ ID | Name | 62.5 pM | 125 pM | 62.5 pM | 125 pM |
| A: VOYHTmiR-127.579 | A: 1599 | VOYPC14 | 0.12 | 0.09 | 0.19 | 0.27 |
| B: VOYHTmiR-104.016 | B: 1589 | | | | | |
| A: VOYHTmiR-104.579 | A: 1595 | VOYPC17 | 0.33 | 0.18 | 0.55 | 0.93 |
| B: VOYHTmiR-104.579 | B: 1595 | | | | | |
| A: VOYHTmiR-127.016 | A: 1593 | VOYPC18 | 0.21 | 0.15 | 0.16 | 0.21 |
| B: VOYHTmiR-127.016 | B: 1593 | | | | | |
| A: VOYHTmiR-104.579 | A: 1595 | VOYPC19 | 0.09 | 0.05 | 0.10 | 0.07 |
| B: VOYHTmiR-127.016 | B: 1593 | | | | | |
| A: VOYHTmiR-127.016 | A: 1593 | VOYPC20 | 0.07 | 0.04 | 0.09 | 0.09 |
| B: VOYHTmiR-104.579 | B: 1595 | | | | | |

The constructs with the VOYHTmiR-127.016 and VOYHTmiR-104.579 modulatory polynucleotides in tandem in any order showed the lowest RLU for both transfection conditions.

### B. Activity of Polycistronic Constructs (62.5 pM and 125 pM) in HeLa at 48 and 72 Hours

The relative expression of HTT mRNA 48 and 72 hours after transfection at 62.5 pM and 125 pM was determined by qRT-PCR for HeLa cells. The relative HTT mRNA expression was obtained by normalizing the HTT mRNA level to the housekeeping gene mRNA level as determined by qRT-PCR; this normalized HTT mRNA level was then expressed relative to the normalized HTT mRNA level in mCherry-treated cells. The results and the description of the constructs tested are shown in Table 56-57. In Table 57, two modulatory polynucleotides were tested in each vector and the modulatory polynucleotides were in tandem. In the table, the vector encodes the A modulatory polynucleotide before the B modulatory polynucleotide.

TABLE 56
Knock-Down of HTT

| | | Relative HTT | |
|---|---|---|---|
| | | mRNA Level (%) | |
| Modulatory | Modulatory | (normalized to Control) | |
| Polynucleotide | polynucleotide | 48 hours | |
| Name | SEQ ID | 62.5 pM | 125 pM |
| VOYHTmiR-104.579 | 1595 | 73 | 91 |
| VOYHTmiR-127.016 | 1593 | 52 | 51 |
| Construct 1: | Construct 1: | 43 | 23 |
| VOYHTmiR-104.579 | 1595 | | |
| Construct 2: | Construct 2: | | |
| VOYHTmiR-127.016 | 1593 | | |

TABLE 57
Knock-Down of HTT

| | | | Relative HTT | |
|---|---|---|---|---|
| | | | mRNA Level (%) | |
| | | Polycistronic | (normalized | |
| | Modulatory | miRNA | to Control) | |
| | polynu- | expression | 62.5 pM | |
| Modulatory Polynu- | cleotide | vector | 48 | 72 |
| cleotide Name | SEQ ID | SEQ ID | Hours | Hours |
| A: VOYHTmiR-104.579 | A: 1595 | VOYPC17 | 97 | 88 |
| B: VOYHTmiR-104.579 | B: 1595 | | | |
| A: VOYHTmiR-127.016 | A: 1593 | VOYPC18 | 36 | 39 |
| B: VOYHTmiR-127.016 | B: 1593 | | | |
| A: VOYHTmiR-104.579 | A: 1595 | VOYPC19 | 37 | 37 |
| B: VOYHTmiR-127.016 | B: 1593 | | | |
| A: VOYHTmiR-127.016 | A: 1593 | VOYPC20 | 49 | 51 |
| B: VOYHTmiR-104.579 | B: 1595 | | | |

The constructs with the VOYHTmiR-127.016 and VOYHTmiR-104.579 modulatory polynucleotides in tandem in any order showed the lowest relative Htt mRNA levels for both time points.

### Example 5. Activity of Polycistronic Constructs in HEK293T Cells

To determine relative activities for inhibiting the target gene, miRNA expression vectors encoding VOYHTmiR-104.016 (SEQ ID NO: 1589) and/or VOYHTmiR-127.579 (SEQ ID NO: 1599) singly or in various tandem combinations comprising two, three or four modulatory polynucleotides were constructed and either transfected into HEK293T cells as plasmids, or packaged in AAV2 and infected into HEK293T cells, and then target gene mRNA levels were measured.

A. Activity of Polycistronic Constructs with Up to 2 Modulatory Polynucleotides after Plasmid Transfection

HEK293T cells were plated into 96-well plates (2.5E4 cells/well in 100 ul cell culture medium) and co-transfected with miRNA expression plasmid (62.5 or 125 pM) and a dual-luciferase plasmid containing the firefly luciferase gene for normalization of transfection efficiency and the VOYHTmiR-104.016 and VOYHTmiR-127.579 target regions of the huntingtin (HTT) gene cloned downstream of the stop codon for the Renilla luciferase gene. At 24 or 36 hours after transfection, the relative activities of the polycistronic constructs for inhibiting the HTT target mRNA were determined by measuring the Renilla and firefly luciferase activities with the Dual-Glo™ Luciferase Assay System, and normalizing the Renilla luciferase activity to the internal control firefly luciferase activity. These normalized Renilla luciferase activities (RLU, relative light units) were then expressed relative to normalized Renilla luciferase activity (average set to 1) in HEK293T cells transfected with control plasmid (pcDNA) at the same concentration.

The relative RLU (mean±standard deviation) for the various constructs and the description of the constructs tested are shown in Table 58 for 24 and 36 hours after transfection. Two constructs, each encoding a single modulatory polynucleotide—either VOYHTmiR-104.016 (SEQ ID NO: 1589) or VOYHTmiR-127.579 (SEQ ID NO: 1599)—served as references for four constructs that each encoded two modulatory polynucleotides (VOYHTmiR-104.016 (SEQ ID NO: 1589) and/or VOYHTmiR-127.579 (SEQ ID NO: 1599)) in tandem, where each polynucleotide is driven by its own H1 promoter and followed by its own H1 terminator. In Table 58, the construct encodes the A modulatory polynucleotide before the B modulatory polynucleotide. N/A means not applicable.

TABLE 58
Polycistronic Activity After Transfection of HEK293T Cells

| Modulatory | Modulatory | | Relative RLU at | | Relative RLU at | |
|---|---|---|---|---|---|---|
| Polynucleotide | polynucleotide | Sequence | 24 Hours | | 36 Hours | |
| Name | SEQ ID | Name | 62.5 pM | 125 pM | 62.5 pM | 125 pM |
| Construct 1: | 1589 (ITR to | N/A | 0.15 ± 0.01 | 0.15 ± 0.01 | 0.07 ± 0.00 | 0.07 ± 0.00 |
| VOYHTmiR-104.016 | ITR sequence: | | | | | |
| | SEQ ID NO: 2691) | | | | | |
| Construct 2: | 1599 (ITR to | N/A | 0.15 ± 0.02 | 0.14 ± 0.01 | 0.08 ± 0.00 | 0.07 ± 0.00 |
| VOYHTmiR-127.579 | ITR sequence: | | | | | |
| | SEQ ID NO: 2690) | | | | | |
| Construct 1: | Construct 1: 1589 | N/A | 0.11 ± 0.01 | 0.09 ± 0.01 | 0.03 ± 0.00 | 0.03 ± 0.00 |
| VOYHTmiR-104.016 | Construct 2: 1599 | | | | | |
| Construct 2: | | | | | | |
| VOYHTmiR-127.579 | | | | | | |
| A: VOYHTmiR-104.016 | A: 1589 | VOYPC59 | 0.08 ± 0.02 | 0.10 ± 0.03 | 0.03 ± 0.00 | 0.03 ± 0.00 |
| B: VOYHTmiR-104.016 | B: 1589 | | | | | |
| A: VOYHTmiR-127.579 | A: 1599 | VOYPC61 | 0.08 ± 0.02 | 0.08 ± 0.02 | 0.03 ± 0.00 | 0.03 ± 0.00 |
| B: VOYHTmiR-104.016 | B: 1589 | | | | | |
| A: VOYHTmiR-104.016 | A: 1589 | VOYPC60 | 0.09 ± 0.02 | 0.10 ± 0.02 | 0.03 ± 0.00 | 0.03 ± 0.00 |
| B: VOYHTmiR-127.579 | B: 1599 | | | | | |
| A: VOYHTmiR-127.579 | A: 1599 | VOYPC62 | 0.15 ± 0.03 | 0.13 ± 0.02 | 0.07 ± 0.00 | 0.07 ± 0.00 |
| B: VOYHTmiR-127.579 | B: 1599 | | | | | |

These results demonstrate that sequences VOYPC59, VOYPC60 and VOYPC61, each containing two modulatory polynucleotides in tandem (either two copies of VOYHTmiR-104.016 (SEQ ID NO: 1589), or a combination of one copy of VOYHTmiR-104.016 (SEQ ID NO: 1589) and one copy of VOYHTmiR-127.579 (SEQ ID NO: 1599)), provide more target lowering than the constructs containing a single modulatory polynucleotide (either VOYHTmiR-104.016 (SEQ ID NO: 1589) or VOYHTmiR-127.579 (SEQ ID NO: 1599)).

B. Activity of Polycistronic Constructs with Up to 4 Modulatory Polynucleotides after Infection with AAV

HEK293T cells were plated into 96-well plates (2.5E4 cells/well in 100 ul cell culture medium), and infected with miRNA expression vectors packaged in AAV2 at an MOI of 1×10³ vector genomes per cell, as well as transfected with a dual-luciferase plasmid containing the firefly luciferase gene for normalization of transfection efficiency and the VOYHTmiR-104.016 and VOYHTmiR-127.579 target regions of the HTT gene cloned downstream of the stop codon for the Renilla luciferase gene. At 48 hours after infection, the relative activities of the constructs for inhibiting the HTT target mRNA were determined by measuring the Renilla and firefly luciferase activities with the Dual-Glo™ Luciferase Assay System, and normalizing the Renilla luciferase activity to the internal control firefly luciferase activity. These normalized Renilla luciferase activities (RLU, relative light units) were then expressed relative to normalized Renilla luciferase activity (average set to 1) in HEK293T cells infected with control vector (AAV2.mCherry) at the same MOI, or uninfected HEK293T cells.

The relative RLU (mean±standard deviation) for the various constructs and the description of the constructs tested are shown in Table 59. Two AAV vectors, each encoding a single modulatory polynucleotide—either VOYHTmiR-104.016 (SEQ ID NO: 1589) or VOYHTmiR-127.579 (SEQ ID NO: 1599)—served as references for sixteen AAV vectors that contained two, three or four modulatory polynucleotides (VOYHTmiR-104.016 (SEQ ID NO: 1589) and/or VOYHTmiR-127.579 (SEQ ID NO: 1599)) in tandem, where each polynucleotide is driven by its own Pol III H1 promoter and followed by its own H1 terminator. In Table 59, the construct encodes the A modulatory polynucleotide before the B modulatory polynucleotide, which in turn is before the C modulatory polynucleotide, which in turn is before the D modulatory polynucleotide. N/A means not applicable.

TABLE 59
Polycistronic Activity After AAV Infection of HEK293T Cells

| Modulatory | Modulatory | | RLU |
|---|---|---|---|
| Polynucleotide | polynucleotide | Sequence | (Relative to |
| Name | SEQ ID | Name | Uninfected) |
| mCherry | N/A | N/A | 0.99 ± 0.02 |
| Uninfected | N/A | N/A | 1.00 ± 0.03 |
| VOYHTmiR-104.016 | 1589 (ITR | N/A | 0.25 ± 0.01 |
| | to ITR sequence: | | |
| | SEQ ID NO: 2691) | | |
| A: VOYHTmiR-104.016 | A: 1589 | VOYPC59 | 0.25 ± 0.01 |
| B: VOYHTmiR-104.016 | B: 1589 | | |
| A: VOYHTmiR-104.016 | A: 1589 | VOYPC33 | 0.21 ± 0.01 |
| B: VOYHTmiR-104.016 | B: 1589 | | |
| C: VOYHTmiR-104.016 | C: 1589 | | |
| A: VOYHTmiR-104.016 | A: 1589 | VOYPC47 | 0.19 ± 0.01 |
| B: VOYHTmiR-104.016 | B: 1589 | | |
| C: VOYHTmiR-104.016 | C: 1589 | | |
| D: VOYHTmiR-104.016 | D: 1589 | | |
| VOYHTmiR-127.579 | 1599 (ITR | N/A | 0.48 ± 0.01 |
| | to ITR sequence: | | |
| | SEQ ID NO: 2690) | | |
| A: VOYHTmiR-127.579 | A: 1599 | VOYPC62 | 0.42 ± 0.01 |
| B: VOYHTmiR-127.579 | B: 1599 | | |
| A: VOYHTmiR-127.579 | A: 1599 | VOYPC31 | 0.34 ± 0.03 |
| B: VOYHTmiR-127.579 | B: 1599 | | |
| C: VOYHTmiR-127.579 | C: 1599 | | |
| A: VOYHTmiR-127.579 | A: 1599 | VOYPC43 | 0.32 ± 0.02 |
| B: VOYHTmiR-127.579 | B: 1599 | | |
| C: VOYHTmiR-127.579 | C: 1599 | | |
| D: VOYHTmiR-127.579 | D: 1599 | | |
| A: VOYHTmiR-104.016 | A: 1589 | VOYPC60 | 0.27 ± 0.01 |
| B: VOYHTmiR-127.579 | B: 1599 | | |
| A: VOYHTmiR-127.579 | A: 1599 | VOYPC61 | 0.24 ± 0.03 |
| B: VOYHTmiR-104.016 | B: 1589 | | |
| A: VOYHTmiR-127.579 | A: 1599 | VOYPC29 | 0.28 ± 0.01 |
| B: VOYHTmiR-104.016 | B: 1589 | | |
| C: VOYHTmiR-127.579 | C: 1599 | | |
| A: VOYHTmiR-104.016 | A: 1589 | VOYPC34 | 0.20 ± 0.00 |
| B: VOYHTmiR-104.016 | B: 1589 | | |
| C: VOYHTmiR-127.579 | C: 1599 | | |
| A: VOYHTmiR-104.016 | A: 1589 | VOYPC30 | 0.26 ± 0.03 |
| B: VOYHTmiR-127.579 | B: 1599 | | |
| C: VOYHTmiR-104.016 | C: 1589 | | |
| A: VOYHTmiR-127.579 | A: 1599 | VOYPC32 | 0.23 ± 0.01 |
| B: VOYHTmiR-127.579 | B: 1599 | | |
| C: VOYHTmiR-104.016 | C: 1589 | | |
| A: VOYHTmiR-127.579 | A: 1599 | VOYPC44 | 0.17 ± 0.01 |
| B: VOYHTmiR-104.016 | B: 1589 | | |
| C: VOYHTmiR-127.579 | C: 1599 | | |
| D: VOYHTmiR-104.016 | D: 1589 | | |
| A: VOYHTmiR-104.016 | A: 1589 | VOYPC48 | 0.20 ± 0.01 |
| B: VOYHTmiR-127.579 | B: 1599 | | |
| C: VOYHTmiR-104.016 | C: 1589 | | |
| D: VOYHTmiR-127.579 | D: 1599 | | |
| A: VOYHTmiR-104.016 | A: 1589 | VOYPC46 | 0.19 ± 0.01 |
| B: VOYHTmiR-127.579 | B: 1599 | | |
| C: VOYHTmiR-127.579 | C: 1599 | | |
| D: VOYHTmiR-104.016 | D: 1589 | | |
| A: VOYHTmiR-127.579 | A: 1599 | VOYPC45 | 0.21 ± 0.01 |
| B: VOYHTmiR-104.016 | B: 1589 | | |
| C: VOYHTmiR-104.016 | C: 1589 | | |
| D: VOYHTmiR-127.579 | D: 1599 | | |

The results show that sequence VOYPC47 which contains 4 identical modulatory polynucleotides (VOYHTmiR-104.016 (SEQ ID NO: 1589)) in tandem provides more target lowering than VOYPC33 which contains 3 of the same modulatory polynucleotides (VOYHTmiR-104.016 (SEQ ID NO: 1589)) in tandem. These results also show that VOYPC33 which contains 3 identical modulatory polynucleotides (VOYHTmiR-104.016 (SEQ ID NO: 1589)) in tandem provides more target lowering than VOYPC59 which contains 2 of the same modulatory polynucleotides (VOYHTmiR-104.016 (SEQ ID NO: 1589)) in tandem.

The results show that sequence VOYPC43 which contains 4 identical modulatory polynucleotides (VOYHTmiR-127.579 (SEQ ID NO: 1599)) in tandem provides more target lowering than VOYPC31 which contains 3 of the same modulatory polynucleotides (VOYHTmiR-127.579 (SEQ ID NO: 1599)) in tandem. These results also show that VOYPC31 which contains 3 identical modulatory polynucleotides (VOYHTmiR-127.579 (SEQ ID NO: 1599)) in tandem provides more target lowering than VOYPC62 which contains 2 of the same modulatory polynucleotides (VOYHTmiR-127.579 (SEQ ID NO: 1599)) in tandem.

Taken together, these results with VOYHTmiR-104.016 (SEQ ID NO: 1589) and with VOYHTmiR-127.579 (SEQ ID NO: 1599) demonstrate that 4 identical modulatory polynucleotides in tandem provides more inhibitor activity (target lowering) than 3 of the same modulatory polynucleotides in tandem, which in turn provides more inhibitor activity (target lowering) than 2 of the same modulatory polynucleotides in tandem.

The results show that sequence VOYPC34, which contains two copies of VOYHTmiR-104.016 (SEQ ID NO: 1589) followed by one copy of VOYHTmiR-127.579 (SEQ ID NO: 1599) provides more inhibitory activity (target lowering) than VOYPC30. Both sequences contain two copies of VOYHTmiR-104.016 (SEQ ID NO: 1589) and one copy of VOYHTmiR-127.579 (SEQ ID NO: 1599), but the order of these modulatory polynucleotides is different; VOYPC34 contains two copies of VOYHTmiR-104.016 (SEQ ID NO: 1589) followed by one copy of VOYHTmiR-127.579 (SEQ ID NO: 1599) whereas VOYPC30 contains one copy of VOYHTmiR-104.016 (SEQ ID NO: 1589) followed by one copy of VOYHTmiR-127.579 (SEQ ID NO: 1599) followed by one copy of VOYHTmiR-104.016 (SEQ ID NO: 1589).

The results show that of the sequences containing four modulatory polynucleotides comprising two different modulatory polynucleotides, sequence VOYPC44 provides more inhibitory activity (target lowering) than VOYPC48, VOYPC46 or VOYPC45.

### Example 6. Pri-miRNA Processing of Polycistronic Constructs in HEK293T Cells

To determine precision and efficiency of pri-miRNA processing, miRNA expression vectors encoding VOYHTmiR-104.016 (SEQ ID NO: 1589) and/or VOYHTmiR-127.579 (SEQ ID NO: 1599) singly or in various tandem combinations comprising two modulatory polynucleotides were constructed, packaged in AAV2 with one CMV promoter, or two H1 promoters, and infected into HEK293T cells, and then precision and efficiency of pri-miRNA processing was assessed by deep sequencing.

HEK293T cells were plated into 6-well plates (2E6 cells/plate in 2 mL cell culture medium), and infected with miRNA expression vectors packaged in AAV2 at an MOI of 1×10⁴ vector genomes per cell, in duplicate (Rep1, Rep2); see Tables 60-65. At 48 hours after infection, the cell cultures were evaluated for pri-miRNA processing by deep sequencing to assess abundance of guide strand relative to the total endogenous pool of miRNAs (Tables 60-61), guide:passenger strand ratio (Tables 62-63), and precision of processing at the 5′-end of the guide strand (Tables 64-65). In Tables 60-65, the construct encodes the A modulatory polynucleotide before the B modulatory polynucleotide. N/A means not applicable.

With the CMV promoter (Table 60), guide strand abundance of VOYHTmiR-104.016 was affected by the presence of a second modulatory polynucleotide in the AAV genome. Guide strand abundance was lower with an AAV genome containing two copies of VOYHTmiR-104.016 (SEQ ID NO: 1589) (VOYPC13, 0.26 and 0.27% relative to the total endogenous miRNA pool) than with an AAV genome containing a single copy of VOYHTmiR-104.016 (SEQ ID NO: 1589) (0.49 and 0.43% relative to the total endogenous miRNA pool). However, guide strand abundance for VOYHTmiR-104.016 was higher with an AAV genome containing a second different modulatory polynucleotide VOYHTmiR-127.579. Guide strand abundance for VOYPC14 was 1.69 and 1.52% relative to the total endogenous miRNA pool, and guide strand abundance for VOYPC15 was 2.17 and 2.11% relative to the total endogenous miRNA pool, in contrast to guide strand abundance with a single modulatory polynucleotide (VOYHTmiR-104.016 (SEQ ID NO: 1589) which was 0.49 and 0.43% relative to the total endogenous miRNA pool. Sequences utilizing CMV promoters were configured with the modulatory polynucleotides in tandem 3′ to a CMV promoter, such that transcription of the modulatory polynucleotides was under the control of a single CMV promoter. Results obtained using the CMV promoter are shown in Table 60.

TABLE 60
Pri-miRNA Processing in HEK293T Cultures after AAV
Infection (CMV Promoter) - Guide Strand Abundance

| | | | Guide Strand Abundance Relative to | | | |
|---|---|---|---|---|---|---|
| Modulatory | Modulatory | | Endogenous miRNA Pool (%) | | | |
| Polynucleotide | polynucleotide | Sequence | 127.579 | | 104.016 | |
| Name | SEQ ID | Name | Rep1 | Rep2 | Rep1 | Rep2 |
| VOYHTmiR-127.579 | 1599 (ITR to | N/A | 1.92 | 0.85 | N/A | N/A |
| | ITR sequence: | | | | | |
| | SEQ ID NO: 2692) | | | | | |
| VOYHTmiR-104.016 | 1589 (ITR to | N/A | N/A | N/A | 0.49 | 0.43 |
| | ITR sequence: | | | | | |
| | SEQ ID NO: 2693) | | | | | |
| Construct 1: | Construct 1: | N/A | 0.98 | 0.72 | 0.24 | 0.23 |
| VOYHTmiR-104.016 | 1589 (ITR to | | | | | |
| Construct 2: | ITR sequence: | | | | | |
| VOYHTmiR-127.579 | SEQ ID NO: 2693) | | | | | |
| | Construct 2: | | | | | |
| | 1599 (ITR to | | | | | |
| | ITR sequence: | | | | | |
| | SEQ ID NO: 2692) | | | | | |
| A: VOYHTmiR-104.016 | A: 1589 | VOYPC13 | N/A | N/A | 0.26 | 0.27 |
| B: VOYHTmiR-104.016 | B: 1589 | | | | | |
| A: VOYHTmiR-127.579 | A: 1599 | VOYPC14 | 0.26 | 0.24 | 1.69 | 1.52 |
| B: VOYHTmiR-104.016 | B: 1589 | | | | | |
| A: VOYHTmiR-104.016 | A: 1589 | VOYPC15 | 0.31 | 0.28 | 2.17 | 2.11 |
| B: VOYHTmiR-127.579 | B: 1599 | | | | | |
| A: VOYHTmiR-127.579 | A: 1599 | VOYPC16 | 0.93 | 0.72 | N/A | N/A |
| B: VOYHTmiR-127.579 | B: 1599 | | | | | |

Sequences utilizing the Pol III promoter H1 were configured with each modulatory polynucleotide under control by its own H1 promoter. As shown in Table 61, with the H1 promoter, guide strand abundance was propoortional to the number of corresponding modulatory polynucleotides in the AAV genome. Guide strand abundance of VOYHTmiR-104.016 (SEQ ID NO: 1589) was 1.77-fold higher with an AAV genome containing two copies of VOYHTmiR-104.016 (VOYPC59, 3.81 and 3.84% relative to the total endogenous miRNA pool) than with an AAV genome containing a single copy of VOYHTmiR-104.016 (2.19 and 2.13% relative to the total endogenous miRNA pool). Guide strand abundance of VOYHTmiR-104.016 (SEQ ID NO: 1589) was similar with an AAV genome containing one copy of VOYHTmiR-104.016 whether or not a copy of a different modulatory polynucleotide VOYHTmiR-127.579 (SEQ ID NO: 1599) was present in the AAV genome. The guide strand abundance relative to the total endogenous miRNA pool of VOYHTmiR-104.016 was 2.19 and 2.13%, 2.61 and 2.52%, and 2.21 and 2.3% with an AAV genome containing one copy of VOYHTmiR-104.016 (SEQ ID NO: 1589), an AAV genome containing one copy of VOYHTmiR-104.016 (SEQ ID NO: 1589) followed by one copy of VOYHTmiR-127.579 (SEQ ID NO: 1599) (VOYPC60), and an AAV genome containing one copy of VOYHTmiR-127.579 (SEQ ID NO: 1599) followed by one copy of VOYHTmiR-104.016 (SEQ ID NO: 1589) (VOYPC61), respectively.

Similarly, with the H1 promoter (Table 61), for another modulatory polynucleotide, guide strand abundance of VOYHTmiR-127.579 (SEQ ID NO: 1599) was 2.67-fold higher with an AAV genome containing two copies of VOYHTmiR-127.579 (VOYPC62, 2.05 and 1.74% relative to the total endogenous miRNA pool) than with an AAV genome containing a single copy of VOYHTmiR-127.579 (0.75 and 0.67% relative to the total endogenous miRNA pool). Guide strand abundance of VOYHTmiR-127.579 (SEQ ID NO: 1599) was similar with an AAV genome containing one copy of VOYHTmiR-127.579 whether or not a copy of a different modulatory polynucleotide VOYHTmiR-104.016 (SEQ ID NO: 1589) was present in the AAV genome. The guide strand abundance of VOYHTmiR-127.579 relative to the total endogenous miRNA pool was 0.75 and 0.67%, 1.0 and 1.05%, and 0.97 and 0.99% with an AAV genome containing one copy of VOYHTmiR-127.579 (SEQ ID NO: 1599), an AAV genome containing one copy of VOYHTmiR-104.016 (SEQ ID NO: 1589) followed by one copy of VOYHTmiR-127.579 (SEQ ID NO: 1599) (VOYPC60), and an AAV genome containing one copy of VOYHTmiR-127.579 (SEQ ID NO: 1599) followed by one copy of VOYHTmiR-104.016 (SEQ ID NO: 1589) (VOYPC61), respectively.

TABLE 61
Pri-miRNA Processing in HEK293T Cultures after AAV
Infection (H1 Promoter) - Guide Strand Abundance

| | | | Guide Strand Abundance | | | |
|---|---|---|---|---|---|---|
| | | | Relative to Endogenous | | | |
| Modulatory | Modulatory | | miRNA Pool (%) | | | |
| Polynucleotide | polynucleotide | Sequence | 127.579 | | 104.016 | |
| Name | SEQ ID | Name | Rep1 | Rep2 | Rep1 | Rep2 |
| VOYHTmiR-127.579 | 1599 (ITR to | N/A | 0.75 | 0.67 | N/A | N/A |
| | ITR sequence: | | | | | |
| | SEQ ID NO: 2690) | | | | | |
| VOYHTmiR-104.016 | 1589 (ITR to | N/A | N/A | N/A | 2.19 | 2.13 |
| | ITR sequence: | | | | | |
| | SEQ ID NO: 2691) | | | | | |
| Construct 1: | Construct 1: | N/A | 0.32 | 0.29 | 1.54 | 1.56 |
| VOYHTmiR-104.016 | 1589 (ITR to | | | | | |
| Construct 2: | ITR sequence: | | | | | |
| VOYHTmiR-127.579 | SEQ ID NO: 2691) | | | | | |
| | Construct 2: | | | | | |
| | 1599 (ITR to | | | | | |
| | ITR sequence: | | | | | |
| | SEQ ID NO: 2690) | | | | | |
| A: VOYHTmiR-104.016 | A: 1589 | VOYPC59 | N/A | N/A | 3.81 | 3.84 |
| B: VOYHTmiR-104.016 | B: 1589 | | | | | |
| A: VOYHTmiR-127.579 | A: 1599 | VOYPC61 | 0.97 | 0.99 | 2.21 | 2.3 |
| B: VOYHTmiR-104.016 | B: 1589 | | | | | |
| A: VOYHTmiR-104.016 | A: 1589 | VOYPC60 | 1 | 1.05 | 2.61 | 2.52 |
| B: VOYHTmiR-127.579 | B: 1599 | | | | | |
| A: VOYHTmiR-127.579 | A: 1599 | VOYPC62 | 2.05 | 1.74 | N/A | N/A |
| B: VOYHTmiR-127.579 | B: 1599 | | | | | |

With the CMV promoter (Table 62), the guide/passenger strand ratio for VOYHTmiR-104.016 was 114.2 and 121.6, and 99.2 and 105.8 for an AAV genome containing one copy of VOYHTmiR-104.016 (SEQ ID NO: 1589) followed by one copy of VOYHTmiR-127.579 (SEQ ID NO: 1599) (VOYPC15), and an AAV genome containing one copy of VOYHTmiR-127.579 (SEQ ID NO: 1599) followed by one copy of VOYHTmiR-104.016 (SEQ ID NO: 1589) (VOYPC14), respectively, versus 71.1 and 83 for an AAV genome containing a single copy of VOYHTmiR-104.016 only.

TABLE 62
Pri-miRNA Processing in HEK293T Cultures after AAV
Infection (CMV Promoter) - Guide/Passenger Ratio

| Modulatory | Modulatory | | Guide/Passenger Ratio | | | |
|---|---|---|---|---|---|---|
| Polynucleotide | polynucleotide | Sequence | 127.579 | | 104.016 | |
| Name | SEQ ID | Name | Rep1 | Rep2 | Rep1 | Rep2 |
| VOYHTmiR-127.579 | 1599 (ITR to | N/A | 16.6 | 4.6 | N/A | N/A |
| | ITR sequence: | | | | | |
| | SEQ ID NO: 2692) | | | | | |
| VOYHTmiR-104.016 | 1589 (ITR to | N/A | N/A | N/A | 71.1 | 83 |
| | ITR sequence: | | | | | |
| | SEQ ID NO: 2693) | | | | | |
| Construct 1: | Construct 1: | N/A | 9.4 | 6 | 45.2 | 66.9 |
| VOYHTmiR-104.016 | 1589 (ITR to | | | | | |
| Construct 2: | ITR sequence: | | | | | |
| VOYHTmiR-127.579 | SEQ ID NO: 2693 | | | | | |
| | Construct 2: | | | | | |
| | 1599 (ITR to | | | | | |
| | ITR sequence: | | | | | |
| | SEQ ID NO: 2692) | | | | | |
| A: VOYHTmiR-104.016 | A: 1589 | VOYPC13 | N/A | N/A | 45.8 | 126.3 |
| B: VOYHTmiR-104.016 | B: 1589 | | | | | |
| A: VOYHTmiR-127.579 | A: 1599 | VOYPC14 | 6.8 | 6.7 | 99.2 | 105.8 |
| B: VOYHTmiR-104.016 | B: 1589 | | | | | |
| A: VOYHTmiR-104.016 | A: 1589 | VOYPC15 | 6.7 | 6.7 | 114.2 | 121.6 |
| B: VOYHTmiR-127.579 | B: 1599 | | | | | |
| A: VOYHTmiR-127.579 | A: 1599 | VOYPC16 | 7.8 | 6.6 | N/A | N/A |
| B: VOYHTmiR-127.579 | B: 1599 | | | | | |

When utilizing the Pol III H1 promoter (Table 63), the guide/passenger ratio of VOYHTmiR-104.016 (SEQ ID NO: 1589) was unaffected by the presence of a second modulatory polynucleotide in the AAV genome. Guide/passenger ratios for VOYHTmiR-104.016 were 16.9 and 20.2, 14.3 and 18.6, 16.3 and 16.3, and 17.7 and 17.8 for an AAV genome containing one copy of VOYHTmiR-104.016 (SEQ ID NO: 1589), an AAV genome containing two copies of VOYHTmiR-104.016 (SEQ ID NO: 1589) (VOYPC59), an AAV genome containing one copy of VOYHTmiR-104.016 (SEQ ID NO: 1589) followed by one copy of VOYHTmiR-127.579 (SEQ ID NO: 1599) (VOYPC60), and an AAV genome containing one copy of VOYHTmiR-127.579 (SEQ ID NO: 1599) followed by one copy of VOYHTmiR-104.016 (SEQ ID NO: 1589) (VOYPC61), respectively.

Similarly, with a Pol III H1 promoter (Table 63), the guide/passenger ratio of VOYHTmiR-127.579 (SEQ ID NO: 1599) was unaffected by the presence of a second modulatory polynucleotide in the AAV genome. Guide/passenger ratios for VOYHTmiR-127.579 were 6.4 and 5.9, 5.7 and 6.4, 5.6 and 6.2, and 6.2 and 5.8 for an AAV genome containing one copy of VOYHTmiR-127.579 (SEQ ID NO: 1599), an AAV genome containing two copies of VOYHTmiR-127.579 (SEQ ID NO: 1599) (VOYPC62), an AAV genome containing one copy of VOYHTmiR-104.016 (SEQ ID NO: 1589) followed by one copy of VOYHTmiR-127.579 (SEQ ID NO: 1599) (VOYPC60), and an AAV genome containing one copy of VOYHTmiR-127.579 (SEQ ID NO: 1599) followed by one copy of VOYHTmiR-104.016 (SEQ ID NO: 1589) (VOYPC61), respectively.

These results demonstrate that with the Pol III H1 promoter (Table 63), the guide/passenger ratio was the same whether a second modulatory polynucleotide was present or not.

TABLE 63
Pri-miRNA Processing in HEK293T Cultures after AAV
Infection (H1 Promoter) - Guide/Passenger Ratio

| Modulatory | Modulatory | | Guide/Passenger Ratio | | | |
|---|---|---|---|---|---|---|
| Polynucleotide | polynucleotide | Sequence | 127.579 | | 104.016 | |
| Name | SEQ ID | Name | Rep1 | Rep2 | Rep1 | Rep2 |
| VOYHTmiR-127.579 | 1599 (ITR to | N/A | 6.4 | 5.9 | N/A | N/A |
| | ITR sequence: | | | | | |
| | SEQ ID NO: 2690) | | | | | |
| VOYHTmiR-104.016 | 1589 (ITR to | N/A | N/A | N/A | 16.9 | 20.2 |
| | ITR sequence: | | | | | |
| | SEQ ID NO: 2691 | | | | | |
| Construct 1: | Construct 1: | N/A | 6.3 | 6 | 17.6 | 19.5 |
| VOYHTmiR-104.016 | 1589 (ITR to | | | | | |
| Construct 2: | ITR sequence: | | | | | |
| VOYHTmiR-127.579 | SEQ ID NO: 2691) | | | | | |
| | Construct 2: | | | | | |
| | 1599 (ITR to | | | | | |
| | ITR sequence: | | | | | |
| | SEQ ID NO: 2690) | | | | | |
| A: VOYHTmiR-104.016 | A: 1589 | VOYPC59 | N/A | N/A | 14.3 | 18.6 |
| B: VOYHTmiR-104.016 | B: 1589 | | | | | |
| A: VOYHTmiR-127.579 | A: 1599 | VOYPC61 | 6.2 | 5.8 | 17.7 | 17.8 |
| B: VOYHTmiR-104.016 | B: 1589 | | | | | |
| A: VOYHTmiR-104.016 | A: 1589 | VOYPC60 | 5.6 | 6.2 | 16.3 | 16.3 |
| B: VOYHTmiR-127.579 | B: 1599 | | | | | |
| A: VOYHTmiR-127.579 | A: 1599 | VOYPC62 | 5.7 | 6.4 | N/A | N/A |
| B: VOYHTmiR-127.579 | B: 1599 | | | | | |

With the CMV promoter (Table 64), the precision of processing at the 5′-end of the guide strand was the same whether or not a second modulatory polynucleotide was present in the AAV genome. With the CMV promoter, the precision of processing at the 5′-end of the guide strand for VOYHTmiR-104.016 (SEQ ID NO: 1589) was 95.5 and 95%, 94.9 and 95.4%, 95.7 and 95.7%, and 95.6 and 95.3% for an AAV genome containing one copy of VOYHTmiR-104.016 (SEQ ID NO: 1589), an AAV genome containing two copies of VOYHTmiR-104.016 (SEQ ID NO: 1589) (VOYPC13), an AAV genome containing one copy of VOYHTmiR-104.016 (SEQ ID NO: 1589) followed by one copy of VOYHTmiR-127.579 (SEQ ID NO: 1599) (VOYPC15), and an AAV genome containing one copy of VOYHTmiR-127.579 (SEQ ID NO: 1599) followed by one copy of VOYHTmiR-104.016 (SEQ ID NO: 1589) (VOYPC14), respectively. With the CMV promoter, the precision of processing at the 5′-end of the guide strand for VOYHTmiR-127.579 (SEQ ID NO: 1599) was 59 and 59.8%, 60.1 and 60.8%, 59.9 and 61.5%, and 61 and 61.2% for an AAV genome containing one copy of VOYHTmiR-127.579 (SEQ ID NO: 1599) an AAV genome containing two copies of VOYHTmiR-127.579 (SEQ ID NO: 1599) (VOYPC16), an AAV genome containing one copy of VOYHTmiR-104.016 (SEQ ID NO: 1589) followed by one copy of VOYHTmiR-127.579 (SEQ ID NO: 1599) (VOYPC15), and an AAV genome containing one copy of VOYHTmiR-127.579 (SEQ ID NO: 1599) followed by one copy of VOYHTmiR-104.016 (SEQ ID NO: 1589) (VOYPC14), respectively.

TABLE 64
Pri-miRNA Processing in HEK293T Cultures after AAV Infection
(CMV Promoter) - Precision of Guide Strand 5′-Processing

| Modulatory | Modulatory | | % N (Guide) | | | |
|---|---|---|---|---|---|---|
| Polynucleotide | polynucleotide | Sequence | 127.579 | | 104.016 | |
| Name | SEQ ID | Name | Rep1 | Rep2 | Rep1 | Rep2 |
| VOYHTmiR-127.579 | 1599 (ITR to | N/A | 59 | 59.8 | N/A | N/A |
| | ITR sequence: | | | | | |
| | SEQ ID NO: 2692) | | | | | |
| VOYHTmiR-104.016 | 1589 (ITR to | N/A | N/A | N/A | 95.5 | 95 |
| | ITR sequence: | | | | | |
| | SEQ ID NO: 2693) | | | | | |
| Construct 1: | Construct 1: | N/A | 58.9 | 60 | 95.3 | 94.6 |
| VOYHTmiR-104.016 | 1589 (ITR to | | | | | |
| Construct 2: | ITR sequence: | | | | | |
| VOYHTmiR-127.579 | SEQ ID NO: 2693) | | | | | |
| | Construct 2: | | | | | |
| | 1599 (ITR to | | | | | |
| | ITR sequence: | | | | | |
| | SEQ ID NO: 2692) | | | | | |
| A: VOYHTmiR-104.016 | A: 1589 | VOYPC13 | N/A | N/A | 94.9 | 95.4 |
| B: VOYHTmiR-104.016 | B: 1589 | | | | | |
| A: VOYHTmiR-127.579 | A: 1599 | VOYPC14 | 61 | 61.2 | 95.6 | 95.3 |
| B: VOYHTmiR-104.016 | B: 1589 | | | | | |
| A: VOYHTmiR-104.016 | A: 1589 | VOYPC15 | 59.9 | 61.5 | 95.7 | 95.7 |
| B: VOYHTmiR-127.579 | B: 1599 | | | | | |
| A: VOYHTmiR-127.579 | A: 1599 | VOYPC16 | 60.1 | 60.8 | N/A | N/A |
| B: VOYHTmiR-127.579 | B: 1599 | | | | | |

With the H1 promoter (Table 65), the precision of processing at the 5′-end of the guide strand was the same whether or not a second modulatory polynucleotide was present in the AAV genome. With the H1 promoter, the precision of processing at the 5′-end of the guide strand for VOYHTmiR-104.016 was 92.6 and 92.6%, 92.6 and 92.1%, 92.1 and 91.8%, and 93 and 92.9% for an AAV genome containing one copy of VOYHTmiR-104.016 (SEQ ID NO: 1589), an AAV genome containing two copies of VOYHTmiR-104.016 (SEQ ID NO: 1589) (VOYPC59), an AAV genome containing one copy of VOYHTmiR-104.016 (SEQ ID NO: 1589) followed by one copy of VOYHTmiR-127.579 (SEQ ID NO: 1599) (VOYPC60), and an AAV genome containing one copy of VOYHTmiR-127.579 (SEQ ID NO: 1599) followed by one copy of VOYHTmiR-104.016 (SEQ ID NO: 1589) (VOYPC61), respectively. With the H1 promoter, the precision of processing at the 5′-end of the guide strand for VOYHTmiR-127.579 (SEQ ID NO: 1599) was 59.5 and 59.6%, 58.5 and 59.3%, 59 and 59.8%, and 58.5 and 58.7% for an AAV genome containing one copy of VOYHTmiR-127.579 (SEQ ID NO: 1599) an AAV genome containing two copies of VOYHTmiR-127.579 (SEQ ID NO: 1599) (VOYPC62), an AAV genome containing one copy of VOYHTmiR-104.016 (SEQ ID NO: 1589) followed by one copy of VOYHTmiR-127.579 (SEQ ID NO: 1599) (VOYPC60), and an AAV genome containing one copy of VOYHTmiR-127.579 (SEQ ID NO: 1599) followed by one copy of VOYHTmiR-104.016 (SEQ ID NO: 1589) (VOYPC61), respectively.

These results demonstrate that with the CMV (Table 64) or H1 (Table 65) promoter, the precision of processing at the 5′-end of the guide strand was the same whether a second modulatory polynucleotide was present or not.

TABLE 65
Pri-miRNA Processing in HEK293T Cultures after AAV Infection
(H1 Promoter) - Precision of Guide Strand 5′-Processing

| Modulatory | Modulatory | | % N (Guide) | | | |
|---|---|---|---|---|---|---|
| Polynucleotide | polynucleotide | Sequence | 127.579 | | 104.016 | |
| Name | SEQ ID | Name | Rep1 | Rep2 | Rep1 | Rep2 |
| VOYHTmiR-127.579 | 1599 (ITR to | N/A | 59.5 | 59.6 | N/A | N/A |
| | ITR sequence: | | | | | |
| | SEQ ID NO: 2690) | | | | | |
| VOYHTmiR-104.016 | 1589 (ITR to | N/A | N/A | N/A | 92.6 | 92.6 |
| | ITR sequence: | | | | | |
| | SEQ ID NO: 2691) | | | | | |
| Construct 1: | Construct 1: | N/A | 59.5 | 60 | 92.6 | 92.4 |
| VOYHTmiR-104.016 | 1589 (ITR to | | | | | |
| Construct 2: | ITR sequence: | | | | | |
| VOYHTmiR-127.579 | SEQ ID NO: 2691) | | | | | |
| | Construct 2: | | | | | |
| | 1599 (ITR to | | | | | |
| | ITR sequence: | | | | | |
| | SEQ ID NO: 2690) | | | | | |
| A: VOYHTmiR-104.016 | A: 1589 | VOYPC59 | N/A | N/A | 92.6 | 92.1 |
| B: VOYHTmiR-104.016 | B: 1589 | | | | | |
| A: VOYHTmiR-127.579 | A: 1599 | VOYPC61 | 58.5 | 58.7 | 93 | 92.9 |
| B: VOYHTmiR-104.016 | B: 1589 | | | | | |
| A: VOYHTmiR-104.016 | A: 1589 | VOYPC60 | 59 | 59.8 | 92.1 | 91.8 |
| B: VOYHTmiR-127.579 | B: 1599 | | | | | |
| A: VOYHTmiR-127.579 | A: 1599 | VOYPC62 | 58.5 | 59.3 | N/A | N/A |
| B: VOYHTmiR-127.579 | B: 1599 | | | | | |

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. An adeno-associated virus (AAV) viral genome comprising a nucleic acid sequence positioned between two inverted terminal repeats (ITRs), wherein said nucleic acid sequence when expressed inhibits or suppresses the expression of a target gene in a cell, wherein said nucleic acid sequence comprises, in a 5′ to 3′ order:
(i) (a) a first 5′ flanking region, a first encoded sense strand sequence, a first loop region, a first encoded antisense strand sequence, and a first 3′ flanking region; or
(b) a first 5′ flanking region, a first encoded antisense strand sequence, a first loop region, a first encoded sense strand sequence, and a first 3′ flanking region, and
(ii) (a) a second 5′ flanking region, a second encoded sense strand, a second loop region, and a second encoded antisense strand sequence; or
(b) a second 5′ flanking region, a second encoded antisense strand sequence, a second loop region, a second encoded sense strand sequence, and a second 3′ flanking region;
wherein:
at least one of the first or second 5′ flanking region comprises the nucleotide sequence of any one of SEQ ID NOs: 1503-1509, 1692, or 1782;
at least one of the first or second loop region comprises the nucleotide sequence of any one of SEQ ID NOs: 1510-1517, or 1693-1694; and/or
at least one of the first or second 3′ flanking region comprises the nucleotide sequence of any one of SEQ ID NOs: 1518-1522, 1695, or 1783.

2.-112. (canceled)

113. The AAV viral genome of claim 1, wherein:
(i) the first encoded antisense strand sequence is complementary to an mRNA of a first target gene; and
(ii) the second encoded antisense strand sequence is complementary to an mRNA of a second target gene.

114. The AAV viral genome of claim 1, wherein the nucleic acid sequence further comprises one or both of:
(i) (a) a third 5′ flanking region, a third encoded sense strand sequence, a third loop region, and a third encoded antisense strand sequence, and a third 3′ flanking region, or
(b) a third 5′ flanking region, a third encoded antisense strand sequence, a third loop region, a third encoded sense strand sequence, and a third 3′ flanking region;
wherein the third encoded antisense strand sequence is complementary to an mRNA of a third target gene; and/or
(ii) (a) a fourth 5′ flanking region, a fourth encoded sense strand sequence, a fourth loop region, a fourth antisense strand sequence, and a fourth 3′ flanking sequence, or
(b) a fourth 5′ flanking region, a fourth encoded antisense strand sequence, a fourth loop region, a fourth encoded sense strand sequence, and a fourth 3′ flanking sequence;
wherein the fourth encoded antisense strand sequence is complementary to an mRNA of a fourth target gene.

115. The AAV viral genome of claim 114, wherein:
(i) at least one of the first, second, third, and fourth 5′ flanking regions comprises the nucleotide sequence of any one of SEQ ID NOs: 1503-1505, 1507, or 1509;
(ii) at least one of the first, second, third and fourth loop regions comprises the nucleotide sequence of any one of SEQ ID NOs: 1510-1513, or 1517; and/or
(iii) wherein at least one of the first, second, third, and fourth 3′ flanking regions comprises the nucleotide sequence of any one of SEQ ID NOs: 1518-1522.

116. The AAV viral genome of claim 114, wherein:
(i) the first target gene is the same as the second target gene; and/or
(ii) the third target gene is the same the first target gene and the second target gene.

117. The AAV viral genome of claim 114, wherein:
(i) the first target gene is not the same as the second target gene; and/or
(ii) the third target gene is the same as the first target gene or is the same as the second target gene.

118. The AAV viral genome of claim 1, wherein the target gene is a huntingtin (HTT) gene or a SOD1 gene.

119. The AAV viral genome of claim 114, wherein:
(i) each sense strand sequence and antisense strand sequence is, independently, 19 to 24 nucleotides in length, 19 to 21 nucleotides in length, 19 nucleotides in length, 20 nucleotides in length, 21 nucleotides in length, or 22 nucleotides in length;
(ii) one or both of the first encoded sense strand sequence and the first encoded antisense strand sequence comprise a 3′ overhang of at least 1 nucleotide or at least 2 nucleotides;
(iii) one or both of the second encoded sense strand sequence and the second encoded antisense strand sequence comprise a 3′ overhang of at least 1 nucleotide or at least 2 nucleotides;
(iv) one or both of the third encoded sense strand sequence and the third encoded antisense strand sequence comprise a 3′ overhang of at least 1 nucleotide or at least 2 nucleotides; and/or
(v) one or both of the fourth encoded sense strand sequence and the fourth encoded antisense strand sequence comprise a 3′ overhang of at least 1 nucleotide or at least 2 nucleotides.

120. The AAV viral genome of claim 114, which further comprises:
(i) a first promoter, which is present 5′ to the first 5′ flanking region;
(ii) a second promoter, which is present 5′ to the second 5′ flanking region;
(iii) a third promoter, which is present 5′ to the third 5′ flanking region; and/or
(iv) a fourth promoter, which is present 5′ to the fourth 5′ flanking region.

121. The AAV viral genome of claim 120, wherein one, two, three, or all of the first, second, third, and fourth promoter is:
(a) a ubiquitous promoter or a cell-type specific promoter;
(b) a CBA promoter, a CMV promoter, a PGK promoter, an H1 promoter, a T7 promoter, a UBC promoter, a GUSB promoter, an NSE promoter, a synapsin promoter, a MeCP2 promoter, or a GFAP promoter.

122. A recombinant adeno-associated virus (AAV) comprising the AAV viral genome of claim 1, and an AAV capsid protein.

123. The recombinant AAV of claim 122, wherein the AAV capsid protein is an AAV9 capsid protein or a variant thereof or an AAV5 capsid protein or a variant thereof.

124. A cell comprising the AAV viral genome of claim 1, wherein the cell is a mammalian cell, an HEK293 cell, an insect cell, an Sf9 cell, a cell of the central nervous system, a neuron, a medium spiny neuron, a motor neuron, or an astrocyte.

125. A pharmaceutical composition comprising the recombinant AAV of claim 122, and a pharmaceutically acceptable excipient.

126. A method of treating a disease of the central nervous system in a subject, comprising administering to the subject an effective amount of the recombinant AAV of claim 122, thereby treating the disease of the central nervous system in the subject.

127. The method of claim 126, wherein the disease of the central nervous system is Huntington's Disease (HD) or ALS.

128. The method of claim 126, wherein the recombinant AAV is administered intravenously, via intracisternal injection, intravascularly, intraventricularly, or via a combination thereof.

129. A method of inhibiting the expression of a target gene in a cell, comprising administering to the cell an effective amount of the recombinant AAV of claim 122, thereby inhibiting expression of the target gene in the cell, optionally wherein:
(i) the target gene is expressed in a neurologic cell, tissue, or organ; and/or
(ii) the cell is a medium spiny neuron, a cortical neuron, a motor neuron, or an astrocyte.

130. The method of claim 129, wherein the cell is in a subject, and the subject has a disease of the central nervous system.

131. A method of producing a recombinant adeno-associated virus (rAAV) comprising providing a cell with a polynucleotide comprising the AAV viral genome of claim 1, at least one polynucleotide encoding AAV rep genes, and at least one polynucleotide encoding AAV cap genes; and harvesting the rAAV from the cell, optionally wherein the cell is a bacterial cell, a mammalian cell, or an insect cell.
